(19) United States
(12) Patent Application Publication
(10) Pub. No.: US 2026/0207618 A1
(43) Pub. Date: Jul. 23, 2026

(54) PYRIMIDINE CARBOXAMIDE COMPOUNDS

(71) Applicant: Acrivon Therapeutics, Inc., Watertown, MA (US)

(72) Inventors: Lars Thomas Brimert, Blentarp (SE); Magnus Munck af Rosenschöld, Lund (SE); John Henry van Duzer, Concord, MA (US); Joon O. Jung, Newton, MA (US); Bo Roger Svensson, Lund (SE)

(21) Appl. No.: 19/143,735

(22) PCT Filed: Dec. 28, 2023
(86) PCT No.: PCT/US23/86278

§ 371 Date: Jun. 26, 2025

Related U.S. Application Data
(60) Provisional application No. 63/436,218, filed on Dec. 30, 2022.

(51) Int. Cl. A61K 31/551; A61K 31/506; A61K 31/5377; A61P 35/00; C07D 239/47; C07D 401/12; C07D 401/14; C07D 403/12; C07D 403/14; C07D 405/12; C07D 471/10

(52) U.S. Cl. CPC A61K 31/551; A61K 31/506; A61K 31/5377; A61P 35/00; C07D 239/47; C07D 401/12; C07D 401/14; C07D 403/12; C07D 403/14; C07D 405/12; C07D 471/10

(57) ABSTRACT

Pyrimidinyl compounds of formula I: (I), for use in methods of inhibiting Wee 1 A kinase.

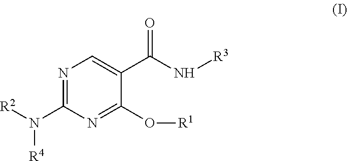

### BACKGROUND

Cells are continuously challenged with endogenous and exogenous agents that influence DNA integrity. To maintain genomic stability and prevent unwanted propagation of damaged DNA, cells have established an organized signaling network that recognizes DNA lesions and halts the cell cycle to allow the DNA to be correctly repaired before resuming DNA replication or cell division. The DNA damage response and the cell cycle are tightly linked via several cell cycle checkpoints that are important control steps for maintaining genomic integrity.

Cancer cells frequently have a defective G1/S checkpoint, often via disrupted p53 activity due to mutations or deletion, or inactivation by viral oncoproteins. Therefore, cancer cells rely heavily on other cell cycle checkpoints, including the G2/M checkpoint, to avoid accumulation of deleterious DNA damage and mitotic catastrophe. As such, cancer cells are hypothesized to be particularly vulnerable to inhibition of proteins that safeguard the entry into mitosis. Matheson, C. J. et al Trends Pharmacol Sci 37, 872-881 (2016).

Wee1A kinase is a tyrosine kinase belonging to the Wee1 kinase family, including Wee1A kinase, Wee1B kinase, and PMYT1. Rora, A. G. L. et al J Hematol Oncol 13, 126 (2020). The primary role for this kinase family is to regulate cell cycle progression and mainly entry into mitosis (Wee1A kinase and PMYT1) or meiosis (Wee1B kinase). The key complex regulating mitotic entry is Cdk1/cyclin B1 complex, also known as the mitosis-promoting factor. Wee1A kinase constrains Cdk1/cyclin B1 complex activity by phosphorylating Cdk1 on the inhibitory tyrosine 15 site (Y¹⁵). Hence, inhibition of Wee1A kinase effectively promotes Cdk1/cyclin B1 complex activity by preventing inhibitory Y¹⁵ phosphorylation. Untimely activation of Cdk1/cyclin B complex promotes premature entry into mitosis with unresolved DNA damages, ultimately leading to mitotic catastrophe and cell death.

In addition to its well-established role in regulating mitotic entry at the G2/M checkpoint, Wee1A kinase has also been suggested to be important in the intra-S checkpoint by limiting activity of Cdk2. Elbok, C. R. et al Cell Reports 38, 110261 (2022); Elbok, C. R. et al Mutat Res Fundam Mol Mech Mutagen 819-820, 111694 (2020). The activity of Cdk2 is regulated by Wee1A kinase in the same way as Cdk1 by tyrosine 15 phosphorylation. Cdk2 is the primary Cdk driving DNA replication and inhibition of Wee1A kinase leads to excessive DNA replication, leading to exhaustion of nucleotide pools and degradation of the ribonucleotide reductase subunit RRM2 (ref). Pfister et al. showed that Wee1A kinase inhibition selectively kills H₃K₃₆me₃-deficient cancer cells through dNTP starvation resulting from RRM2 depletion. Pfister, S. X. et al Cancer Cell 28, 557-568 (2015). The histone methyl transferase SETD2 catalyzes H₃K₃₆me₃, which promotes RRM2 expression and synthesis of dNTPs. Inactivation of SETD2 gene is frequent in clear cell renal carcinomas (ccRCC) and might therefore be sensitive to Wee1A kinase inhibition. A phase II trial is testing AZD1775 in SETD2-deficient solid tumors (NCT03284385).

Wee1A kinase has also been suggested to have a role in controlling histone stoichiometry by phosphorylation of core histone H₂B at tyrosine 37 at late S phase. Koh, S.-B. Cell Signal 94, 110310 (2022).

Cancers associated with high-risk human papilloma virus (HPV) such as head and neck squamous cell carcinoma (HNSCC) showed increased sensitivity to Wee1A kinase inhibition. Diab, A. et al Proc National Acad Sci 117, 28287-28296 (2020). A phase II study of Wee1A kinase inhibitor AZD1775 (Adavosertib) has shown promising results in women with uterine serous carcinoma. Liu, J. F. et al J Clin Oncol 39, 1531-1539 (2021). Inhibition of Wee1A kinase has also shown effect compared to active monitoring in RAS/TP53 mutated metastatic colorectal cancer. Seligmann, J. F. et al J Clin Oncol 39, 3705-3715 (2021).

Several Wee1A kinase inhibitors are currently being tested in clinical trials (Bukhari, A. B. et al Frontiers Oncol 12, 828684 (2022), but there is still a need to additional Wee1A kinase inhibitors with good potency and specificity.

### SUMMARY

In some embodiments, the present disclosure provides a compound of formula I:

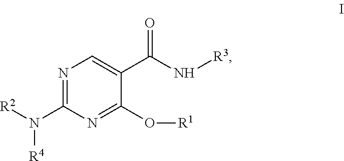

or a pharmaceutically acceptable salt thereof, wherein each of R¹, R², R³, and R⁴ are as defined below and described herein.

In some embodiments, the present disclosure provides a pharmaceutical composition comprising a compound of formula I, or a pharmaceutically acceptable salt thereof, and a pharmaceutically acceptable carrier.

In some embodiments, the present disclosure provides a method of inhibiting Wee1A kinase in a patient or in a biological sample, the method comprising administering to the patient or contacting the biological sample with a compound of formula I, or a pharmaceutically acceptable salt thereof.

In some embodiments, the present disclosure provides a method of treating a disease or disorder associated with Wee1A kinase, the method comprising administering to a patient in need thereof a compound of formula I, or a pharmaceutically acceptable salt thereof.

In some embodiments, the present disclosure provides a method of treating a disease or disorder associated with Wee1A kinase, the method comprising administering to a patient in need thereof a compound of formula I, or a pharmaceutically acceptable salt thereof.

In some such embodiments, the disease or disorder associated with Wee1A kinase is a cancer. In some embodiments, a cancer is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

### DETAILED DESCRIPTION

### 1. General Description of Compounds of the Disclosure

In some embodiments, the present disclosure provides inhibitors of Wee1A kinase. In some embodiments, such compounds include those of the formulae described herein, or a pharmaceutically acceptable salt thereof, wherein each variable is as defined and described herein.

In some embodiments, the present disclosure provides a compound having structural formula I:

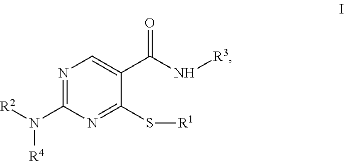

or a solvate, enantiomer, tautomer, or diastereomer thereof, or a pharmaceutically acceptable salt of any of the foregoing, wherein:
R¹ is an optionally substituted C₁-C₆ alkyl or an optionally substituted C₃-C₆ cycloalkyl;
R² is an optionally substituted aryl or an optionally substituted heteroaryl, wherein two substituents on the aryl or heteroaryl are optionally taken together to form a saturated ring fused to the aryl or heteroaryl;
R³ is an optionally substituted aryl or an optionally substituted heteroaryl other than tetrazolyl; and
R⁴ is hydrogen or C₁-C₄ alkyl, wherein the compound is other than:

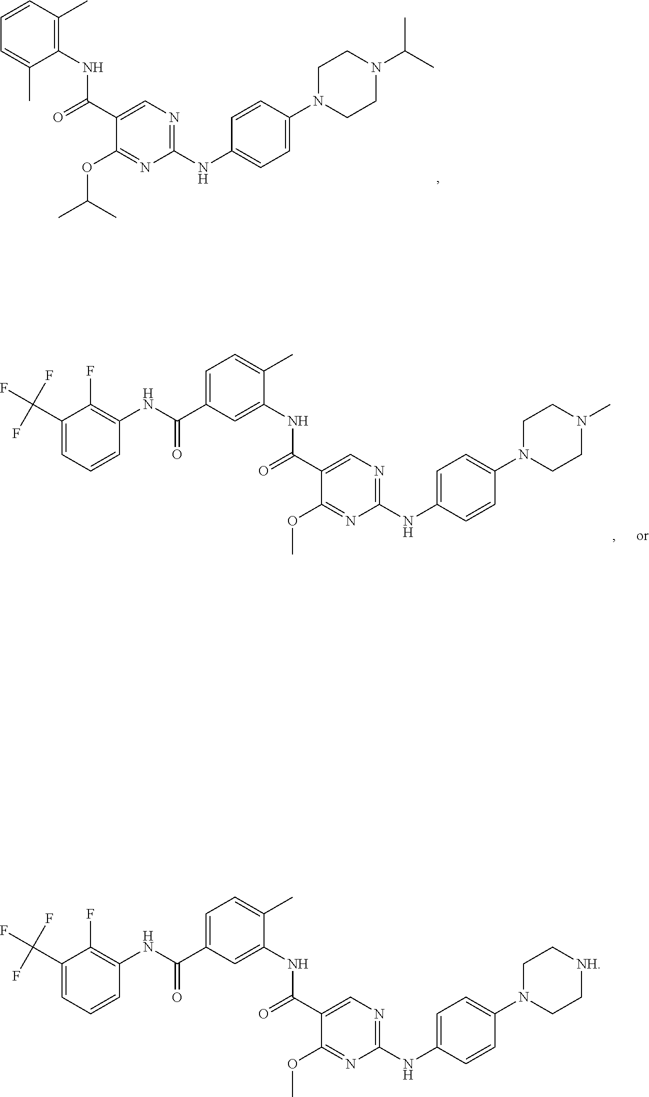 [SMILES: Cc1cccc(C)c1NC(=O)c1cnc(Nc2ccc(N3CCN(C(C)C)CC3)cc2)nc1OC(C)C ; COc1nc(Nc2ccc(N3CCN(C)CC3)cc2)ncc1C(=O)Nc1cc(C(=O)Nc2cccc(C(F)(F)F)c2F)ccc1C ; COc1nc(Nc2ccc(N3CCNCC3)cc2)ncc1C(=O)Nc1cc(C(=O)Nc2cccc(C(F)(F)F)c2F)ccc1C]

In some embodiments, the compound of formula I may be a compound, or a pharmaceutically acceptable salt thereof, selected from Table 1:

TABLE 1
Exemplary Compounds

| # | Structure |
|---|---|
| 100 | 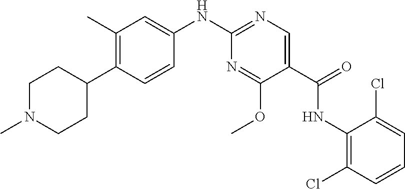 [SMILES: COc1nc(Nc2ccc(C3CCN(C)CC3)c(C)c2)ncc1C(=O)Nc1c(Cl)cccc1Cl] |
| 101 | 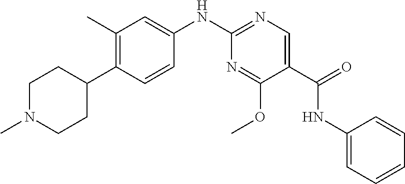 [SMILES: COc1nc(Nc2ccc(C3CCN(C)CC3)c(C)c2)ncc1C(=O)Nc1ccccc1] |
| 102 | 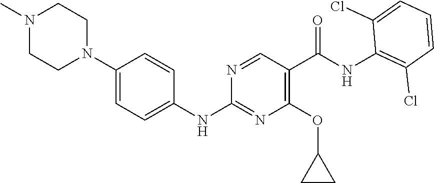 [SMILES: CN1CCN(c2ccc(Nc3ncc(C(=O)Nc4c(Cl)cccc4Cl)c(OC4CC4)n3)cc2)CC1] |
| 103 | 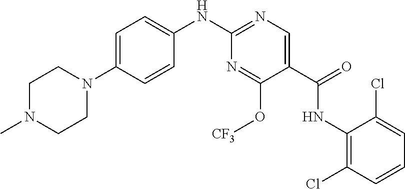 [SMILES: CN1CCN(c2ccc(Nc3ncc(C(=O)Nc4c(Cl)cccc4Cl)c(OC(F)(F)F)n3)cc2)CC1] |
| 104 | 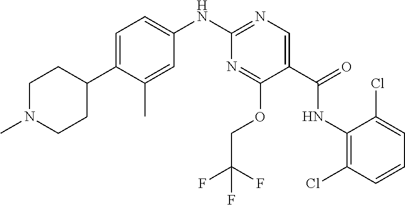 [SMILES: Cc1cc(Nc2ncc(C(=O)Nc3c(Cl)cccc3Cl)c(OCC(F)(F)F)n2)ccc1C1CCN(C)CC1] |
| 105 | 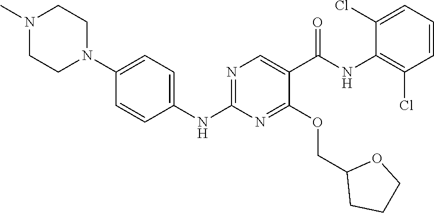 [SMILES: CN1CCN(c2ccc(Nc3ncc(C(=O)Nc4c(Cl)cccc4Cl)c(OCC4CCCO4)n3)cc2)CC1] |
| 106 | 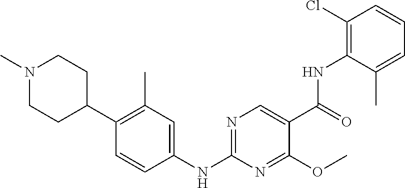 [SMILES: COc1nc(Nc2ccc(C3CCN(C)CC3)c(C)c2)ncc1C(=O)Nc1c(C)cccc1Cl] |
| 107 | 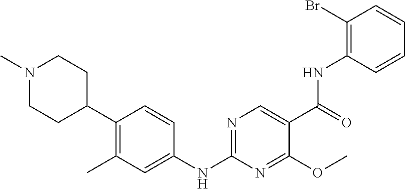 [SMILES: COc1nc(Nc2ccc(C3CCN(C)CC3)c(C)c2)ncc1C(=O)Nc1ccccc1Br] |
| 108 | 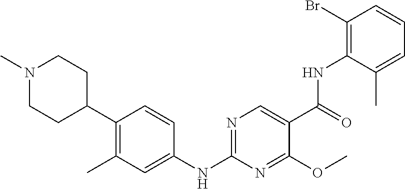 [SMILES: COc1nc(Nc2ccc(C3CCN(C)CC3)c(C)c2)ncc1C(=O)Nc1c(C)cccc1Br] |
| 109 | 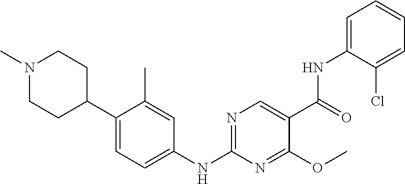 [SMILES: COc1nc(Nc2ccc(C3CCN(C)CC3)c(C)c2)ncc1C(=O)Nc1ccccc1Cl] |
| 110 | 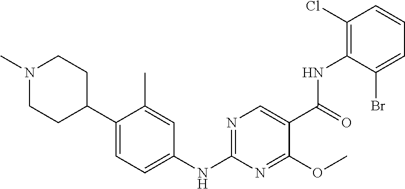 [SMILES: COc1nc(Nc2ccc(C3CCN(C)CC3)c(C)c2)ncc1C(=O)Nc1c(Cl)cccc1Br] |
| 111 | 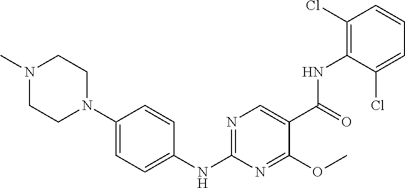 [SMILES: COc1nc(Nc2ccc(N3CCN(C)CC3)cc2)ncc1C(=O)Nc1c(Cl)cccc1Cl] |
| 112 | 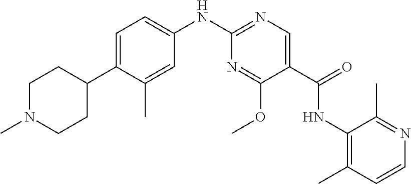 [SMILES: COc1nc(Nc2ccc(C3CCN(C)CC3)c(C)c2)ncc1C(=O)Nc1c(C)ccnc1C] |
| 113 | 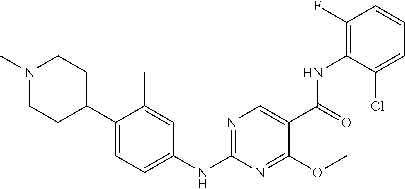 [SMILES: COc1nc(Nc2ccc(C3CCN(C)CC3)c(C)c2)ncc1C(=O)Nc1c(F)cccc1Cl] |
| 114 | 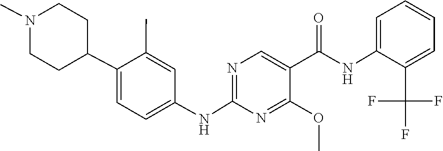 [SMILES: COc1nc(Nc2ccc(C3CCN(C)CC3)c(C)c2)ncc1C(=O)Nc1ccccc1C(F)(F)F] |
| 115 | 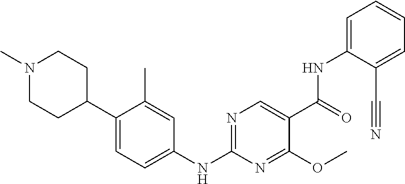 [SMILES: COc1nc(Nc2ccc(C3CCN(C)CC3)c(C)c2)ncc1C(=O)Nc1ccccc1C#N] |
| 116 | 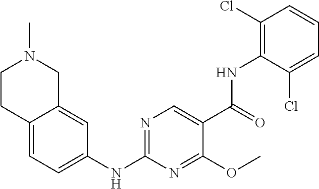 [SMILES: COc1nc(Nc2ccc3c(c2)CN(C)CC3)ncc1C(=O)Nc1c(Cl)cccc1Cl] |
| 117 | 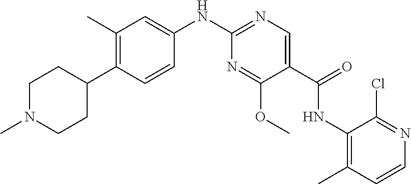 [SMILES: COc1nc(Nc2ccc(C3CCN(C)CC3)c(C)c2)ncc1C(=O)Nc1c(C)ccnc1Cl] |
| 118 | 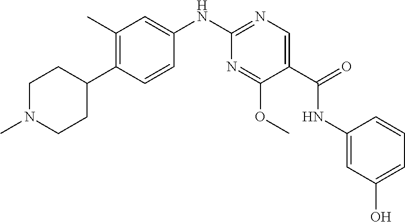 [SMILES: COc1nc(Nc2ccc(C3CCN(C)CC3)c(C)c2)ncc1C(=O)Nc1cccc(O)c1] |
| 119 | 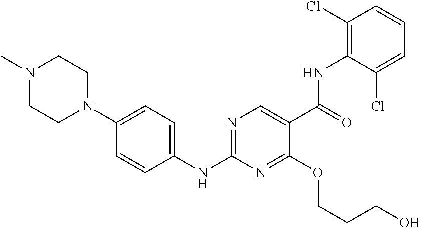 [SMILES: CN1CCN(c2ccc(Nc3ncc(C(=O)Nc4c(Cl)cccc4Cl)c(OCCCO)n3)cc2)CC1] |
| 120 | 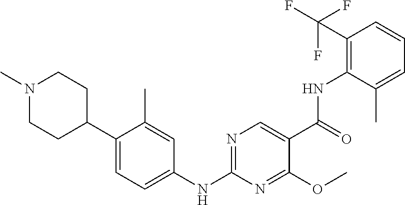 [SMILES: COc1nc(Nc2ccc(C3CCN(C)CC3)c(C)c2)ncc1C(=O)Nc1c(C)cccc1C(F)(F)F] |
| 121 | 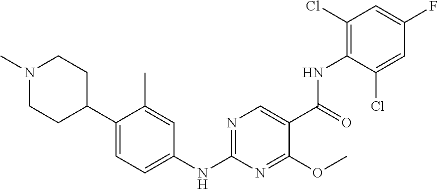 [SMILES: COc1nc(Nc2ccc(C3CCN(C)CC3)c(C)c2)ncc1C(=O)Nc1c(Cl)cc(F)cc1Cl] |
| 122 | 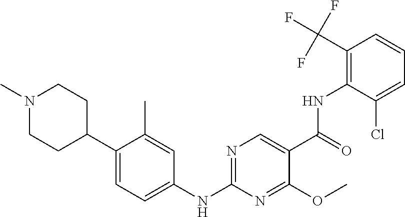 [SMILES: COc1nc(Nc2ccc(C3CCN(C)CC3)c(C)c2)ncc1C(=O)Nc1c(Cl)cccc1C(F)(F)F] |
| 123 | 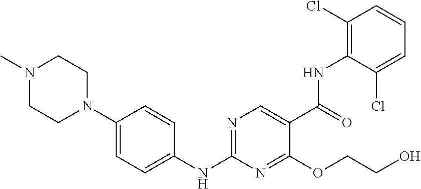 [SMILES: CN1CCN(c2ccc(Nc3ncc(C(=O)Nc4c(Cl)cccc4Cl)c(OCCO)n3)cc2)CC1] |
| 124 | 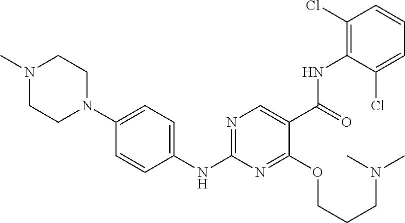 |
| 125 | 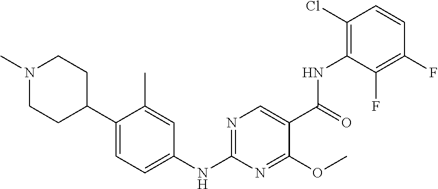 [SMILES: COc1nc(Nc2ccc(C3CCN(C)CC3)c(C)c2)ncc1C(=O)Nc1c(Cl)ccc(F)c1F] |
| 126 | 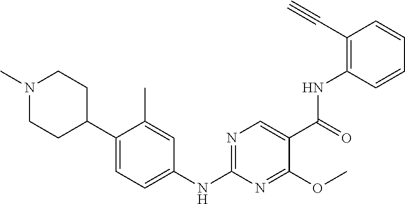 [SMILES: C#Cc1ccccc1NC(=O)c1cnc(Nc2ccc(C3CCN(C)CC3)c(C)c2)nc1OC] |
| 127 | 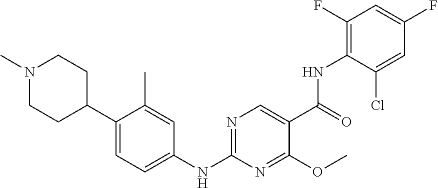 [SMILES: COc1nc(Nc2ccc(C3CCN(C)CC3)c(C)c2)ncc1C(=O)Nc1c(F)cc(F)cc1Cl] |
| 128 | 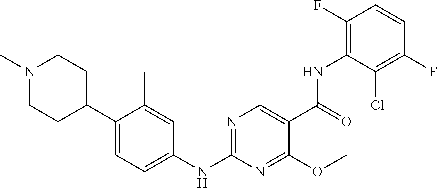 [SMILES: COc1nc(Nc2ccc(C3CCN(C)CC3)c(C)c2)ncc1C(=O)Nc1c(F)ccc(F)c1Cl] |
| 129 | 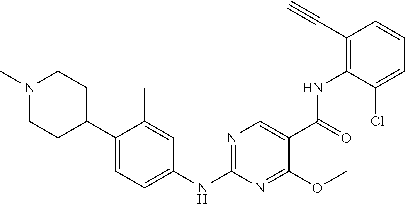 [SMILES: C#Cc1cccc(Cl)c1NC(=O)c1cnc(Nc2ccc(C3CCN(C)CC3)c(C)c2)nc1OC] |
| 130 | 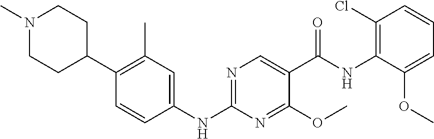 |
| 131 | 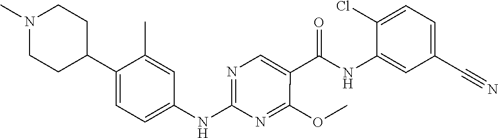 [SMILES: COc1nc(Nc2ccc(C3CCN(C)CC3)c(C)c2)ncc1C(=O)Nc1cc(C#N)ccc1Cl] |
| 132 | 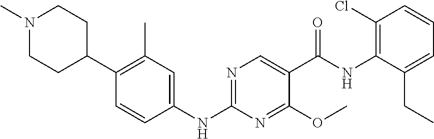 [SMILES: CCc1cccc(Cl)c1NC(=O)c1cnc(Nc2ccc(C3CCN(C)CC3)c(C)c2)nc1OC] |
| 133 | 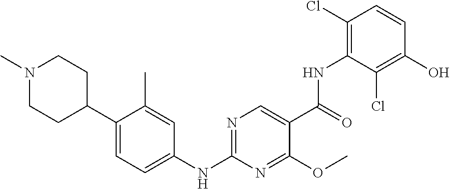 [SMILES: COc1nc(Nc2ccc(C3CCN(C)CC3)c(C)c2)ncc1C(=O)Nc1c(Cl)ccc(O)c1Cl] |
| 134 | 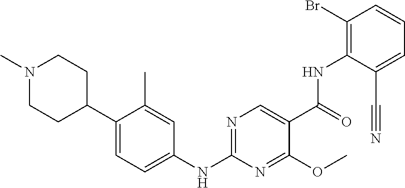 [SMILES: COc1nc(Nc2ccc(C3CCN(C)CC3)c(C)c2)ncc1C(=O)Nc1c(Br)cccc1C#N] |
| 135 | 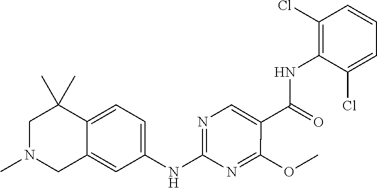 [SMILES: COc1nc(Nc2ccc3c(c2)CN(C)CC3(C)C)ncc1C(=O)Nc1c(Cl)cccc1Cl] |
| 136 | 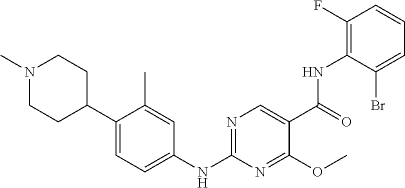 [SMILES: COc1nc(Nc2ccc(C3CCN(C)CC3)c(C)c2)ncc1C(=O)Nc1c(F)cccc1Br] |
| 137 | 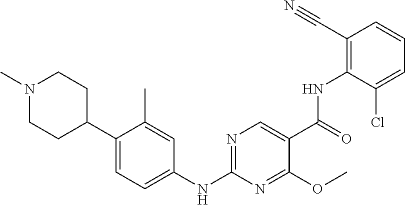 [SMILES: COc1nc(Nc2ccc(C3CCN(C)CC3)c(C)c2)ncc1C(=O)Nc1c(Cl)cccc1C#N] |
| 138 | 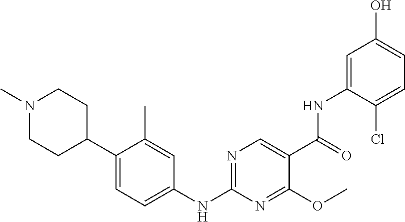 [SMILES: COc1nc(Nc2ccc(C3CCN(C)CC3)c(C)c2)ncc1C(=O)Nc1cc(O)ccc1Cl] |
| 139 | 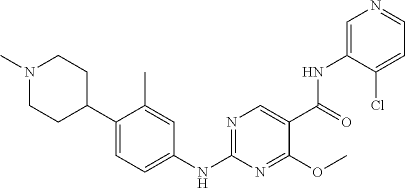 [SMILES: COc1nc(Nc2ccc(C3CCN(C)CC3)c(C)c2)ncc1C(=O)Nc1cnccc1Cl] |
| 140 | 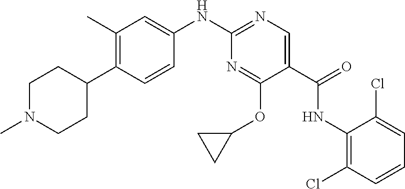 [SMILES: Cc1cc(Nc2ncc(C(=O)Nc3c(Cl)cccc3Cl)c(OC3CC3)n2)ccc1C1CCN(C)CC1] |
| 141 | 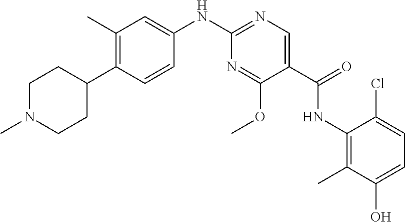 [SMILES: COc1nc(Nc2ccc(C3CCN(C)CC3)c(C)c2)ncc1C(=O)Nc1c(Cl)ccc(O)c1C] |
| 142 | 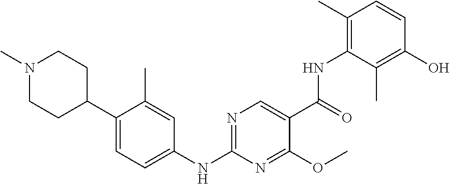 [SMILES: COc1nc(Nc2ccc(C3CCN(C)CC3)c(C)c2)ncc1C(=O)Nc1c(C)ccc(O)c1C] |
| 143 | 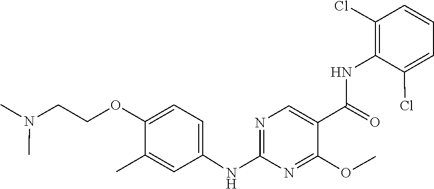 [SMILES: COc1nc(Nc2ccc(OCCN(C)C)c(C)c2)ncc1C(=O)Nc1c(Cl)cccc1Cl] |
| 144 | 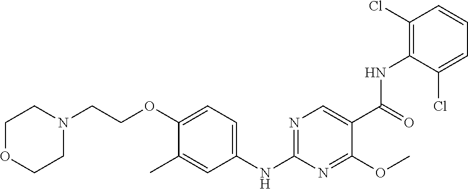 [SMILES: COc1nc(Nc2ccc(OCCN3CCOCC3)c(C)c2)ncc1C(=O)Nc1c(Cl)cccc1Cl] |
| 145 | 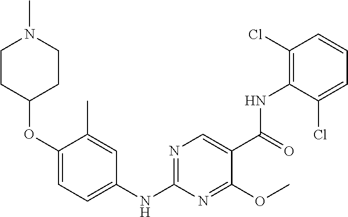 [SMILES: COc1nc(Nc2ccc(OC3CCN(C)CC3)c(C)c2)ncc1C(=O)Nc1c(Cl)cccc1Cl] |
| 146 | 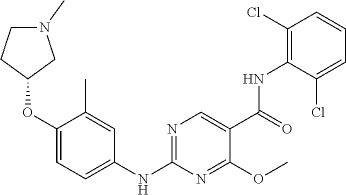 [SMILES: COc1nc(Nc2ccc(O[C@@H]3CCN(C)C3)c(C)c2)ncc1C(=O)Nc1c(Cl)cccc1Cl] |
| 147 | 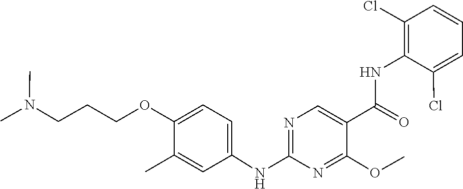 [SMILES: COc1nc(Nc2ccc(OCCCN(C)C)c(C)c2)ncc1C(=O)Nc1c(Cl)cccc1Cl] |
| 148 | 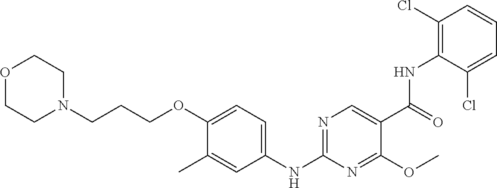 [SMILES: COc1nc(Nc2ccc(OCCCN3CCOCC3)c(C)c2)ncc1C(=O)Nc1c(Cl)cccc1Cl] |
| 149 | 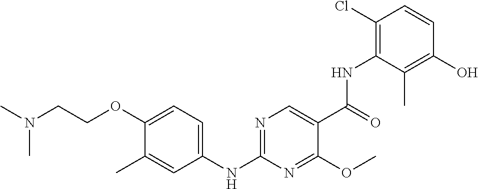 [SMILES: COc1nc(Nc2ccc(OCCN(C)C)c(C)c2)ncc1C(=O)Nc1c(Cl)ccc(O)c1C] |
| 150 | 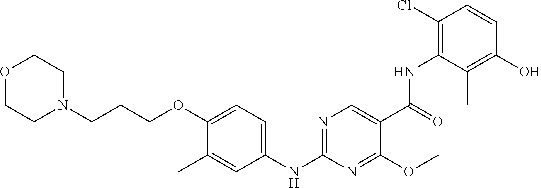 [SMILES: COc1nc(Nc2ccc(OCCCN3CCOCC3)c(C)c2)ncc1C(=O)Nc1c(Cl)ccc(O)c1C] |
| 151 | 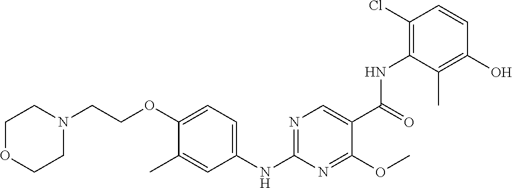 [SMILES: COc1nc(Nc2ccc(OCCN3CCOCC3)c(C)c2)ncc1C(=O)Nc1c(Cl)ccc(O)c1C] |
| 152 | 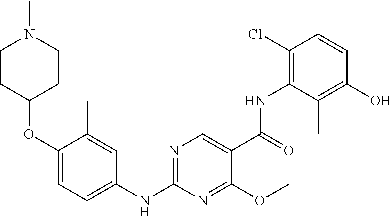 [SMILES: COc1nc(Nc2ccc(OC3CCN(C)CC3)c(C)c2)ncc1C(=O)Nc1c(Cl)ccc(O)c1C] |
| 153 | 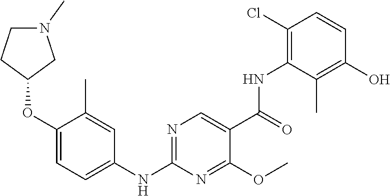 [SMILES: COc1nc(Nc2ccc(O[C@@H]3CCN(C)C3)c(C)c2)ncc1C(=O)Nc1c(Cl)ccc(O)c1C] |
| 154 | 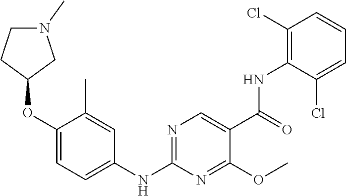 [SMILES: COc1nc(Nc2ccc(O[C@H]3CCN(C)C3)c(C)c2)ncc1C(=O)Nc1c(Cl)cccc1Cl] |
| 155 | 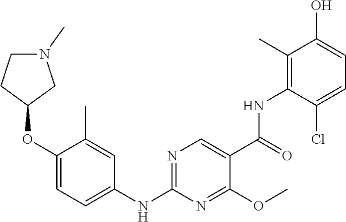 [SMILES: COc1nc(Nc2ccc(O[C@H]3CCN(C)C3)c(C)c2)ncc1C(=O)Nc1c(Cl)ccc(O)c1C] |
| 156 | 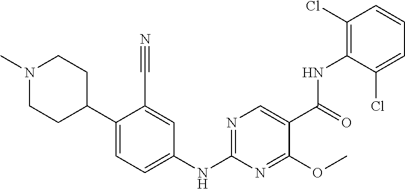 [SMILES: COc1nc(Nc2ccc(C3CCN(C)CC3)c(C#N)c2)ncc1C(=O)Nc1c(Cl)cccc1Cl] |
| 157 | 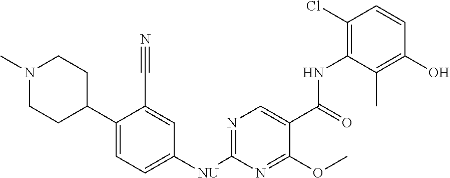 |
| 158 | 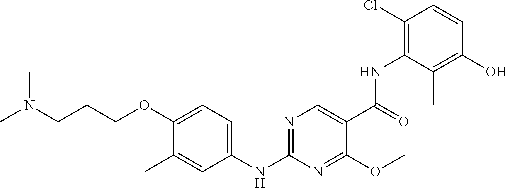 [SMILES: COc1nc(Nc2ccc(OCCCN(C)C)c(C)c2)ncc1C(=O)Nc1c(Cl)ccc(O)c1C] |
| 159 | 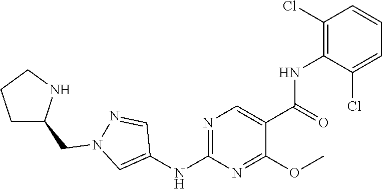 [SMILES: COc1nc(Nc2cnn(C[C@H]3CCCN3)c2)ncc1C(=O)Nc1c(Cl)cccc1Cl] |
| 160 | 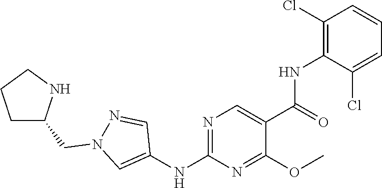 [SMILES: COc1nc(Nc2cnn(C[C@@H]3CCCN3)c2)ncc1C(=O)Nc1c(Cl)cccc1Cl] |
| 161 | 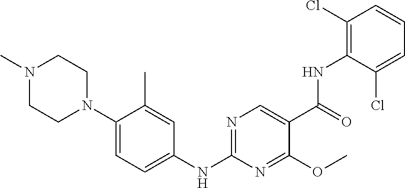 [SMILES: COc1nc(Nc2ccc(N3CCN(C)CC3)c(C)c2)ncc1C(=O)Nc1c(Cl)cccc1Cl] |
| 162 | 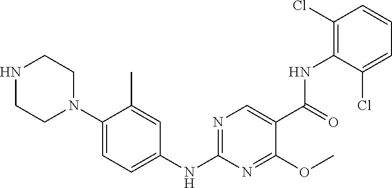 [SMILES: COc1nc(Nc2ccc(N3CCNCC3)c(C)c2)ncc1C(=O)Nc1c(Cl)cccc1Cl] |
| 163 | 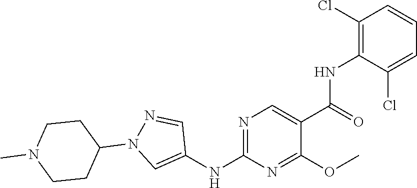 [SMILES: COc1nc(Nc2cnn(C3CCN(C)CC3)c2)ncc1C(=O)Nc1c(Cl)cccc1Cl] |
| 164 | 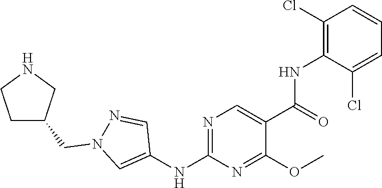 [SMILES: COc1nc(Nc2cnn(C[C@@H]3CCNC3)c2)ncc1C(=O)Nc1c(Cl)cccc1Cl] |
| 165 | 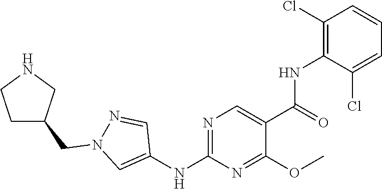 [SMILES: COc1nc(Nc2cnn(C[C@H]3CCNC3)c2)ncc1C(=O)Nc1c(Cl)cccc1Cl] |
| 166 | 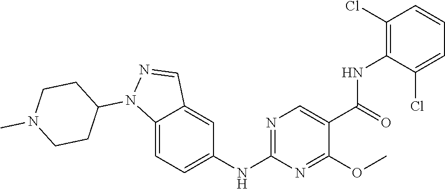 [SMILES: COc1nc(Nc2ccc3c(cnn3C3CCN(C)CC3)c2)ncc1C(=O)Nc1c(Cl)cccc1Cl] |
| 167 | 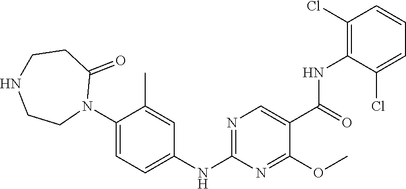 [SMILES: COc1nc(Nc2ccc(N3CCNCCC3=O)c(C)c2)ncc1C(=O)Nc1c(Cl)cccc1Cl] |
| 168 | 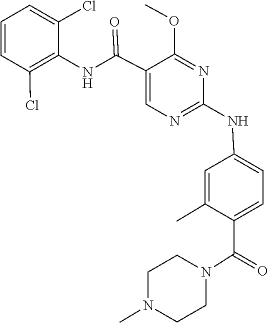 [SMILES: COc1nc(Nc2ccc(C(=O)N3CCN(C)CC3)c(C)c2)ncc1C(=O)Nc1c(Cl)cccc1Cl] |
| 169 | 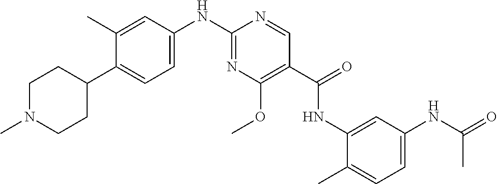 [SMILES: COc1nc(Nc2ccc(C3CCN(C)CC3)c(C)c2)ncc1C(=O)Nc1cc(NC(C)=O)ccc1C] |
| 170 | 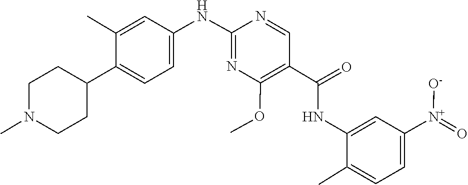 [SMILES: COc1nc(Nc2ccc(C3CCN(C)CC3)c(C)c2)ncc1C(=O)Nc1cc([N+](=O)[O-])ccc1C] |
| 171 | 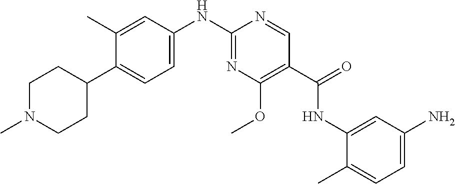 [SMILES: COc1nc(Nc2ccc(C3CCN(C)CC3)c(C)c2)ncc1C(=O)Nc1cc(N)ccc1C] |
| 172 | 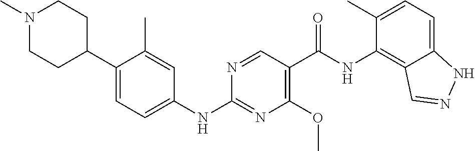 [SMILES: COc1nc(Nc2ccc(C3CCN(C)CC3)c(C)c2)ncc1C(=O)Nc1c(C)ccc2[nH]ncc12] |
| 173 | 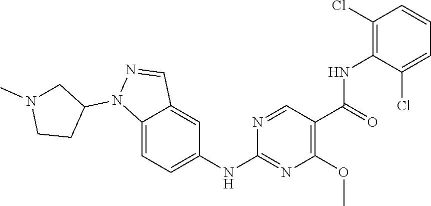 [SMILES: COc1nc(Nc2ccc3c(cnn3C3CCN(C)C3)c2)ncc1C(=O)Nc1c(Cl)cccc1Cl] |
| 174 | 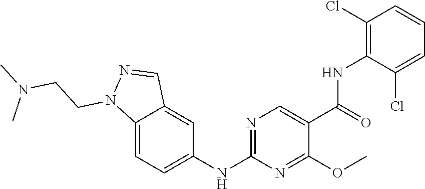 [SMILES: COc1nc(Nc2ccc3c(cnn3CCN(C)C)c2)ncc1C(=O)Nc1c(Cl)cccc1Cl] |
| 175 | 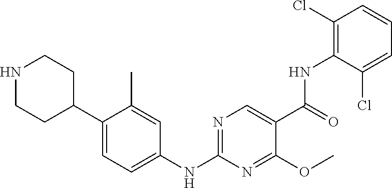 [SMILES: COc1nc(Nc2ccc(C3CCNCC3)c(C)c2)ncc1C(=O)Nc1c(Cl)cccc1Cl] |
| 176 | 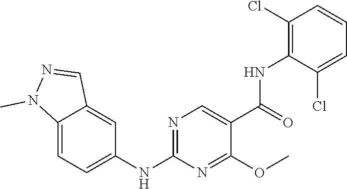 [SMILES: COc1nc(Nc2ccc3c(cnn3C)c2)ncc1C(=O)Nc1c(Cl)cccc1Cl] |
| 177 | 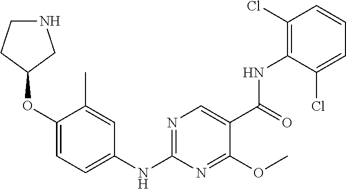 [SMILES: COc1nc(Nc2ccc(O[C@H]3CCNC3)c(C)c2)ncc1C(=O)Nc1c(Cl)cccc1Cl] |
| 178 | 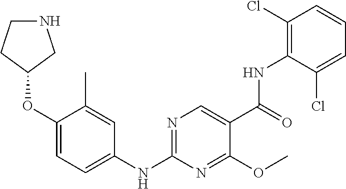 [SMILES: COc1nc(Nc2ccc(O[C@@H]3CCNC3)c(C)c2)ncc1C(=O)Nc1c(Cl)cccc1Cl] |
| 179 | 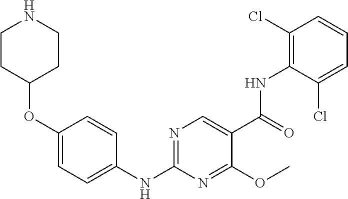 [SMILES: COc1nc(Nc2ccc(OC3CCNCC3)cc2)ncc1C(=O)Nc1c(Cl)cccc1Cl] |
| 180 | 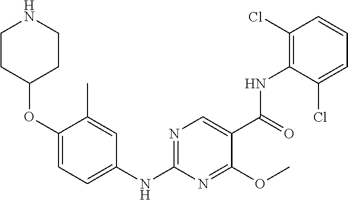 [SMILES: COc1nc(Nc2ccc(OC3CCNCC3)c(C)c2)ncc1C(=O)Nc1c(Cl)cccc1Cl] |
| 181 | 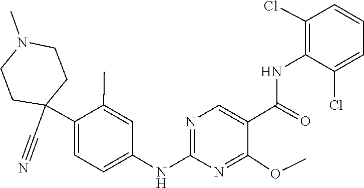 [SMILES: COc1nc(Nc2ccc(C3(C#N)CCN(C)CC3)c(C)c2)ncc1C(=O)Nc1c(Cl)cccc1Cl] |
| 182 | 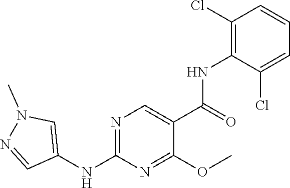 [SMILES: COc1nc(Nc2cnn(C)c2)ncc1C(=O)Nc1c(Cl)cccc1Cl] |
| 183 | 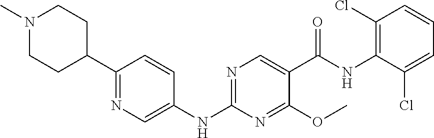 [SMILES: COc1nc(Nc2ccc(C3CCN(C)CC3)nc2)ncc1C(=O)Nc1c(Cl)cccc1Cl] |
| 184 | 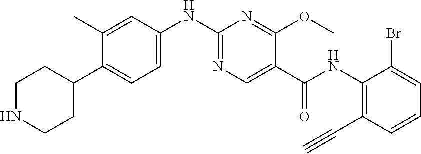 [SMILES: C#Cc1cccc(Br)c1NC(=O)c1cnc(Nc2ccc(C3CCNCC3)c(C)c2)nc1OC] |
| 185 | 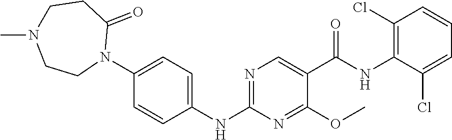 [SMILES: COc1nc(Nc2ccc(N3CCN(C)CCC3=O)cc2)ncc1C(=O)Nc1c(Cl)cccc1Cl] |
| 186 | 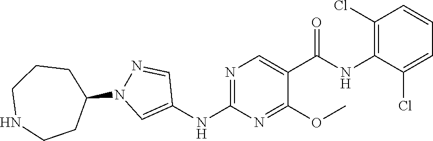 [SMILES: COc1nc(Nc2cnn([C@@H]3CCCNCC3)c2)ncc1C(=O)Nc1c(Cl)cccc1Cl] |
| 187 | 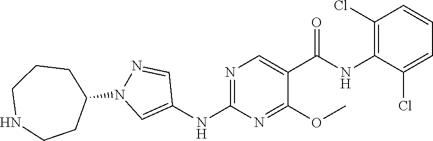 [SMILES: COc1nc(Nc2cnn([C@H]3CCCNCC3)c2)ncc1C(=O)Nc1c(Cl)cccc1Cl] |
| 188 | 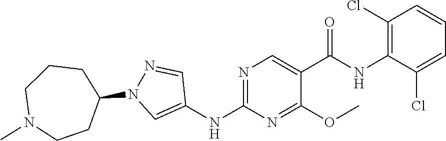 [SMILES: COc1nc(Nc2cnn([C@@H]3CCCN(C)CC3)c2)ncc1C(=O)Nc1c(Cl)cccc1Cl] |
| 189 | 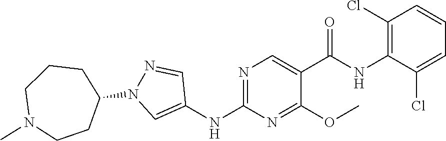 [SMILES: COc1nc(Nc2cnn([C@H]3CCCN(C)CC3)c2)ncc1C(=O)Nc1c(Cl)cccc1Cl] |
| 190 | 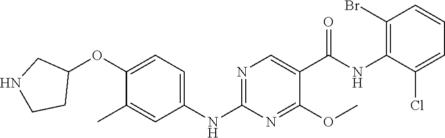 [SMILES: COc1nc(Nc2ccc(OC3CCNC3)c(C)c2)ncc1C(=O)Nc1c(Cl)cccc1Br] |
| 191 | 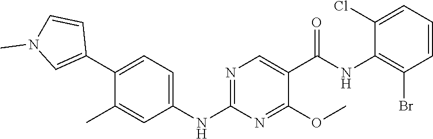 [SMILES: COc1nc(Nc2ccc(-c3ccn(C)c3)c(C)c2)ncc1C(=O)Nc1c(Cl)cccc1Br] |
| 192 | 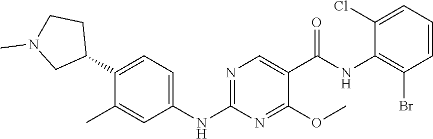 [SMILES: COc1nc(Nc2ccc([C@H]3CCN(C)C3)c(C)c2)ncc1C(=O)Nc1c(Cl)cccc1Br] |
| 193 | 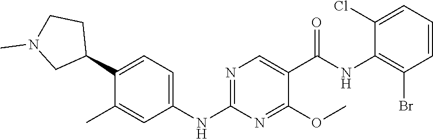 [SMILES: COc1nc(Nc2ccc([C@@H]3CCN(C)C3)c(C)c2)ncc1C(=O)Nc1c(Cl)cccc1Br] |
| 194 | 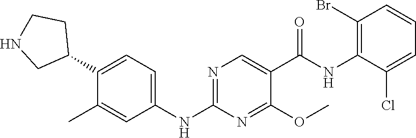 [SMILES: COc1nc(Nc2ccc([C@H]3CCNC3)c(C)c2)ncc1C(=O)Nc1c(Cl)cccc1Br] |
| 195 | 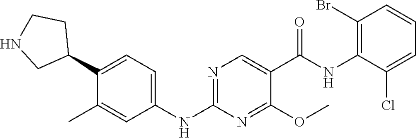 [SMILES: COc1nc(Nc2ccc([C@@H]3CCNC3)c(C)c2)ncc1C(=O)Nc1c(Cl)cccc1Br] |
| 196 | 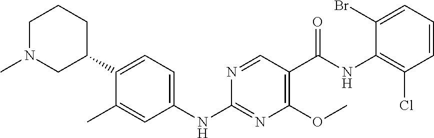 [SMILES: COc1nc(Nc2ccc([C@H]3CCCN(C)C3)c(C)c2)ncc1C(=O)Nc1c(Cl)cccc1Br] |
| 197 | 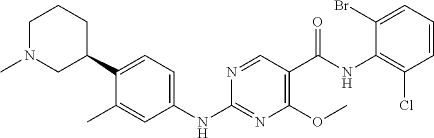 [SMILES: COc1nc(Nc2ccc([C@@H]3CCCN(C)C3)c(C)c2)ncc1C(=O)Nc1c(Cl)cccc1Br] |
| 198 | 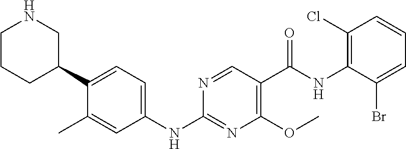 [SMILES: COc1nc(Nc2ccc([C@H]3CCCNC3)c(C)c2)ncc1C(=O)Nc1c(Cl)cccc1Br] |
| 199 | 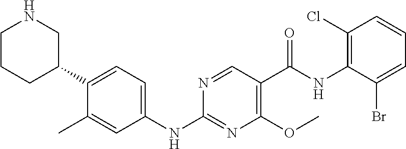 [SMILES: COc1nc(Nc2ccc([C@@H]3CCCNC3)c(C)c2)ncc1C(=O)Nc1c(Cl)cccc1Br] |
| 200 | 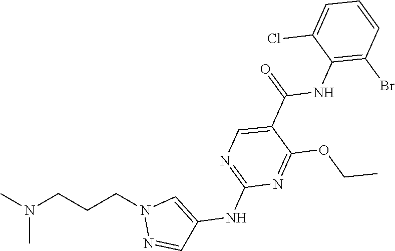 [SMILES: CCOc1nc(Nc2cnn(CCCN(C)C)c2)ncc1C(=O)Nc1c(Cl)cccc1Br] |
| 201 | 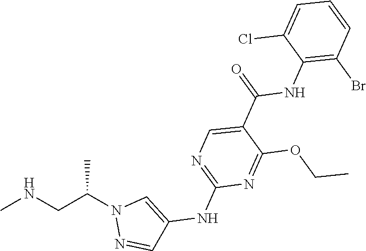 [SMILES: CCOc1nc(Nc2cnn([C@@H](C)CNC)c2)ncc1C(=O)Nc1c(Cl)cccc1Br] |
| 202 | 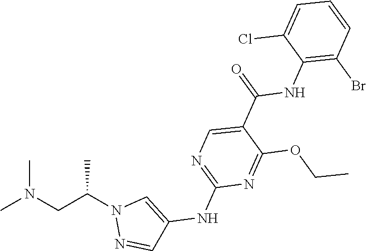 [SMILES: CCOc1nc(Nc2cnn([C@@H](C)CN(C)C)c2)ncc1C(=O)Nc1c(Cl)cccc1Br] |
| 203 | 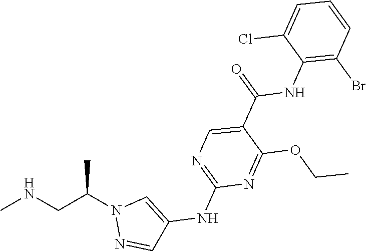 [SMILES: CCOc1nc(Nc2cnn([C@H](C)CNC)c2)ncc1C(=O)Nc1c(Cl)cccc1Br] |
| 204 | 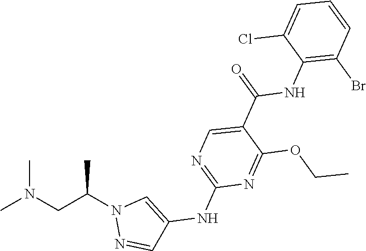 [SMILES: CCOc1nc(Nc2cnn([C@H](C)CN(C)C)c2)ncc1C(=O)Nc1c(Cl)cccc1Br] |
| 205 | 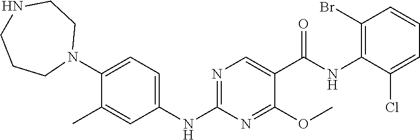 [SMILES: COc1nc(Nc2ccc(N3CCCNCC3)c(C)c2)ncc1C(=O)Nc1c(Cl)cccc1Br] |
| 206 | 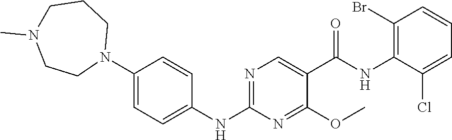 [SMILES: COc1nc(Nc2ccc(N3CCCN(C)CC3)cc2)ncc1C(=O)Nc1c(Cl)cccc1Br] |
| 207 | 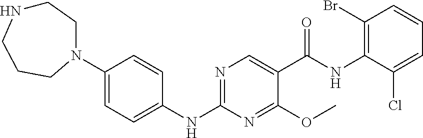 [SMILES: COc1nc(Nc2ccc(N3CCCNCC3)cc2)ncc1C(=O)Nc1c(Cl)cccc1Br] |
| 208 | 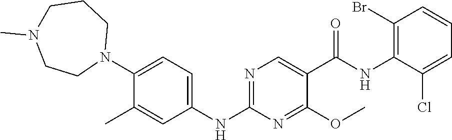 [SMILES: COc1nc(Nc2ccc(N3CCCN(C)CC3)c(C)c2)ncc1C(=O)Nc1c(Cl)cccc1Br] |
| 209 | 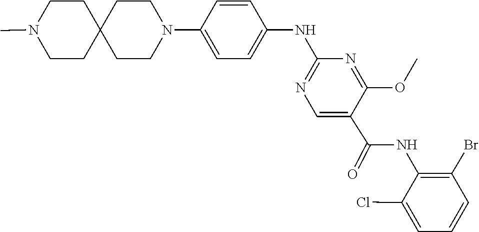 [SMILES: COc1nc(Nc2ccc(N3CCC4(CCN(C)CC4)CC3)cc2)ncc1C(=O)Nc1c(Cl)cccc1Br] |
| 210 | 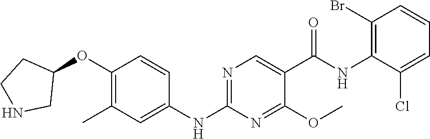 [SMILES: COc1nc(Nc2ccc(O[C@@H]3CCNC3)c(C)c2)ncc1C(=O)Nc1c(Cl)cccc1Br] |
| 211 | 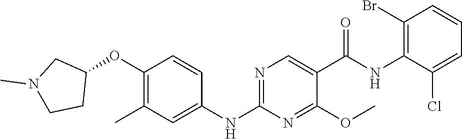 [SMILES: COc1nc(Nc2ccc(O[C@@H]3CCN(C)C3)c(C)c2)ncc1C(=O)Nc1c(Cl)cccc1Br] |
| 212 | 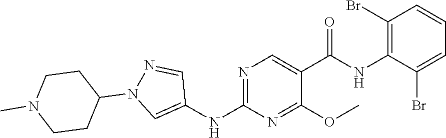 [SMILES: COc1nc(Nc2cnn(C3CCN(C)CC3)c2)ncc1C(=O)Nc1c(Br)cccc1Br] |
| 213 | 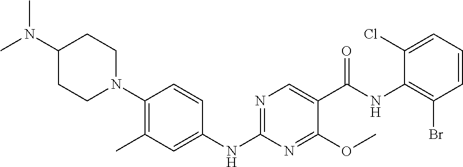 [SMILES: COc1nc(Nc2ccc(N3CCC(N(C)C)CC3)c(C)c2)ncc1C(=O)Nc1c(Cl)cccc1Br] |
| 214 | 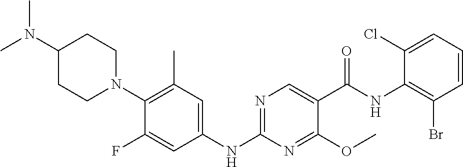 [SMILES: COc1nc(Nc2cc(C)c(N3CCC(N(C)C)CC3)c(F)c2)ncc1C(=O)Nc1c(Cl)cccc1Br] |
| 215 | 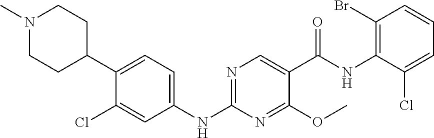 [SMILES: COc1nc(Nc2ccc(C3CCN(C)CC3)c(Cl)c2)ncc1C(=O)Nc1c(Cl)cccc1Br] |
| 216 | 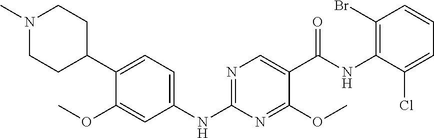 [SMILES: COc1cc(Nc2ncc(C(=O)Nc3c(Cl)cccc3Br)c(OC)n2)ccc1C1CCN(C)CC1] |
| 217 | 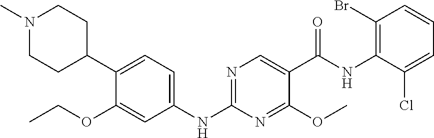 [SMILES: CCOc1cc(Nc2ncc(C(=O)Nc3c(Cl)cccc3Br)c(OC)n2)ccc1C1CCN(C)CC1] |
| 218 | 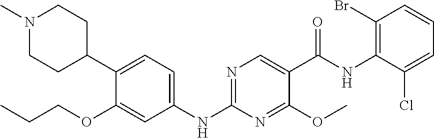 [SMILES: CCCOc1cc(Nc2ncc(C(=O)Nc3c(Cl)cccc3Br)c(OC)n2)ccc1C1CCN(C)CC1] |
| 219 | 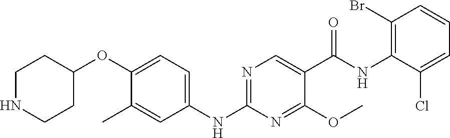 [SMILES: COc1nc(Nc2ccc(OC3CCNCC3)c(C)c2)ncc1C(=O)Nc1c(Cl)cccc1Br] |
| 220 | 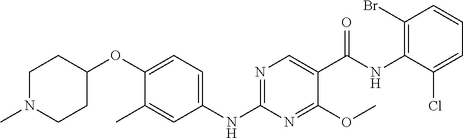 [SMILES: COc1nc(Nc2ccc(OC3CCN(C)CC3)c(C)c2)ncc1C(=O)Nc1c(Cl)cccc1Br] |
| 221 | 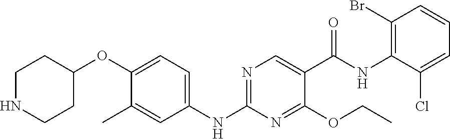 [SMILES: CCOc1nc(Nc2ccc(OC3CCNCC3)c(C)c2)ncc1C(=O)Nc1c(Cl)cccc1Br] |
| 222 | 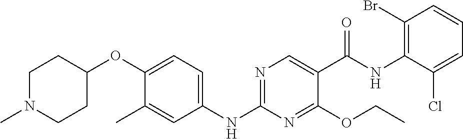 [SMILES: CCOc1nc(Nc2ccc(OC3CCN(C)CC3)c(C)c2)ncc1C(=O)Nc1c(Cl)cccc1Br] |
| 223 | 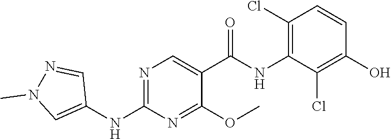 [SMILES: COc1nc(Nc2cnn(C)c2)ncc1C(=O)Nc1c(Cl)ccc(O)c1Cl] |
| 224 | 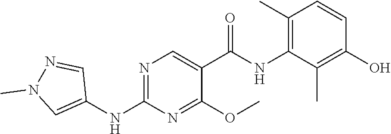 [SMILES: COc1nc(Nc2cnn(C)c2)ncc1C(=O)Nc1c(C)ccc(O)c1C] |
| 225 | 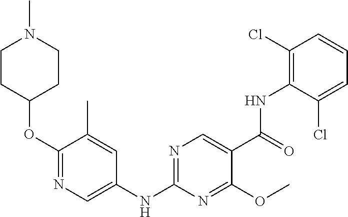 [SMILES: COc1nc(Nc2cnc(OC3CCN(C)CC3)c(C)c2)ncc1C(=O)Nc1c(Cl)cccc1Cl] |
| 226 | 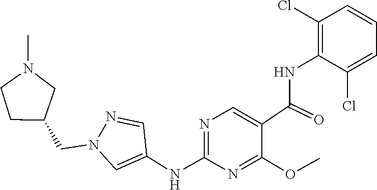 [SMILES: COc1nc(Nc2cnn(C[C@@H]3CCN(C)C3)c2)ncc1C(=O)Nc1c(Cl)cccc1Cl] |
| 227 | 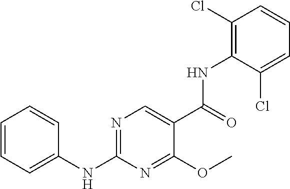 [SMILES: COc1nc(Nc2ccccc2)ncc1C(=O)Nc1c(Cl)cccc1Cl] |
| 228 | 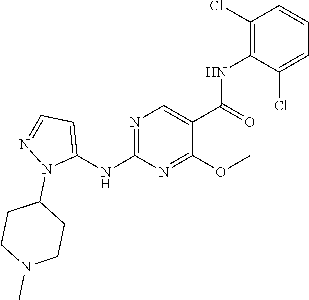 [SMILES: COc1nc(Nc2ccnn2C2CCN(C)CC2)ncc1C(=O)Nc1c(Cl)cccc1Cl] |

or a pharmaceutically acceptable salt thereof.

### 2. Compounds and Definitions

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocyclyl” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein each ring atom is carbon, at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic carbocyclic rings.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridinyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl or heteroaryl rings such that the resulting bi- or multicyclic ring system as a whole is fully aromatic. Nonlimiting examples include indolyl, isoindolyl, benzothienyl, benzofuranyl, dibenzofuranyl, indazolyl, benzimidazolyl, benzothiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, and phenoxazinyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen atom. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepanyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄₀(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}2; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}2; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-7-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S. Suitable divalent substituents on a saturated carbon atom of R^{∘} when R^{∘} is a 5-7-membered saturated, or partially unsaturated ring having 0-4 heteroatoms independently selected from nitrogen, oxygen, and sulfur also include a 4-7 membered saturated, partially unsaturated, or aryl ring having 0-4 heteroatoms independently selected from nitrogen, oxygen, and sulfur. In such instances, the divalent substituent and R^{∘} form a spiro-fused ring.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(Rf)S(O)₂R^{†}; wherein each R^{†} is independently hydrogen, C₁₋₆ aliphatic which may be substituted as defined below, unsubstituted -OPh, or a substituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group and the substituted 5-6-membered saturated, partially unsaturated, or aryl ring having 0-4 heteroatoms independently selected from nitrogen, oxygen, and sulfur of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, rotational isomers (atropisomers) and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Inhibition of activity of a protein kinase, for example, Wee1A kinase or a mutant thereof, in a biological sample is useful for a variety of purposes that are known to one of skill in the art. Examples of such purposes include, but are not limited to, blood transfusion, organ transplantation, biological specimen storage, and biological assays.

As used herein, a “disease or disorder associated with Wee1A kinase” or, alternatively, “a Wee1A kinase-mediated disease or disorder” means any disease or other deleterious condition in which Wee1A kinase, or a mutant thereof, is known or suspected to play a role.

The term “subject”, as used herein, means a mammal and includes human and animal subjects, such as domestic animals (e.g., horses, dogs, cats, etc.). The terms “subject” and “patient” are used interchangeably. In some embodiments, the “patient” or “subject” means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds of the present disclosure that maybe combined with the carrier materials to produce a composition in a single dosage form will vary depending upon the host treated, the particular mode of administration, etc. Preferably, provided compositions are formulated so that a dosage of between 0.01 to about 100 mg/kg, or about 0.1 mg/kg to about 50 mg/kg, and preferably from about 1 mg/kg to about 25 mg/kg, of subject body weight/day of the inhibitor can be administered to a patient receiving these compositions to obtain the desired therapeutic effect. The amount of a compound of the present disclosure in the composition will also depend upon the particular compound in the composition.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

As used herein, the term “inhibitor” is defined as a compound that binds to and/or inhibits the target protein kinase with measurable affinity. In certain embodiments, an inhibitor has an IC₅₀ and/or binding constant of less about 5 μM, less than about 1 μM, less than about 500 nM, less than about 100 nM, less than about 50 nM, or less than about 10 nM.

The terms “measurable affinity” and “measurably inhibit,” as used herein, means a measurable change in Wee1A kinase activity between a sample comprising a compound of the present disclosure, or composition thereof, and an equivalent sample comprising Wee1A kinase, in the absence of said compound, or composition thereof.

The term “aryl ring having 0-4 heteroatoms independently selected from nitrogen, oxygen, and sulfur” as used herein means an aryl ring when such ring has 0 heteroatoms or a heteroaryl ring when such ring has 1-4 heteroatoms.

### 3. Description of Exemplary Compounds

In some embodiments, the present disclosure provides a compound having structural formula I:

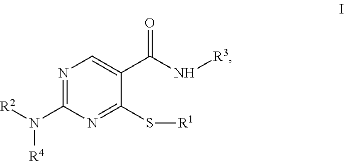

or a solvate, enantiomer, tautomer, or diastereomer thereof, or a pharmaceutically acceptable salt of any of the foregoing, wherein:
R¹ is an optionally substituted C₁-C₆ alkyl or an optionally substituted C₃-C₆ cycloalkyl;
R² is an optionally substituted aryl or an optionally substituted heteroaryl, wherein two substituents on the aryl or heteroaryl are optionally taken together to form a saturated ring fused to the aryl or heteroaryl;
R³ is an optionally substituted aryl or an optionally substituted heteroaryl other than tetrazolyl; and
R⁴ is hydrogen or C₁-C₄ alkyl, wherein the compound is other than:

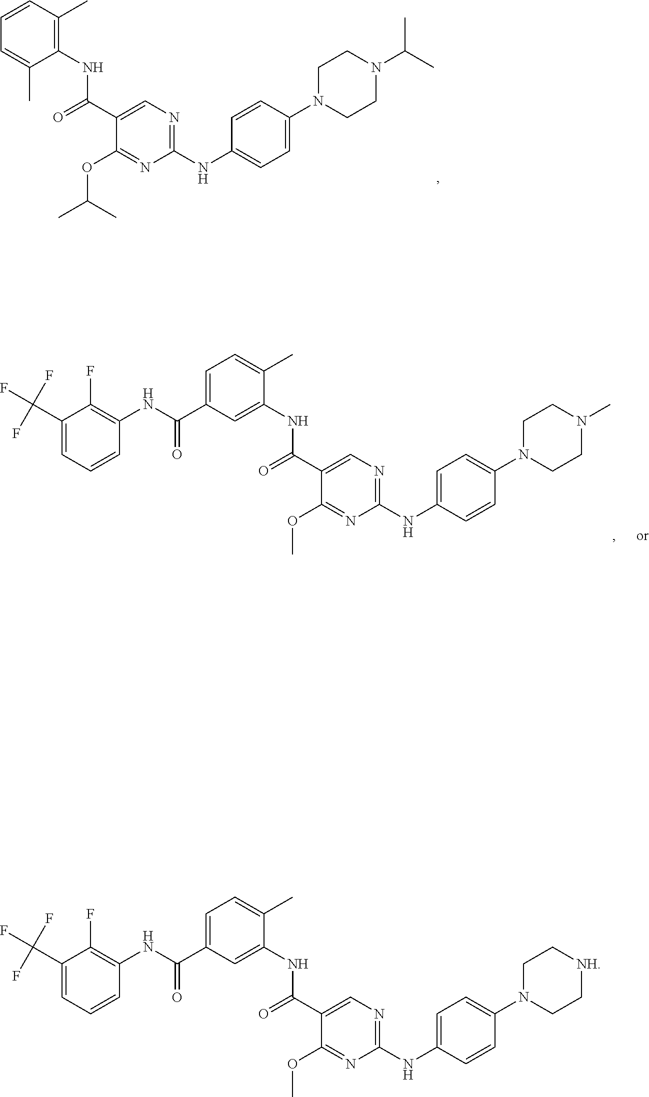 [SMILES: Cc1cccc(C)c1NC(=O)c1cnc(Nc2ccc(N3CCN(C(C)C)CC3)cc2)nc1OC(C)C ; COc1nc(Nc2ccc(N3CCN(C)CC3)cc2)ncc1C(=O)Nc1cc(C(=O)Nc2cccc(C(F)(F)F)c2F)ccc1C ; COc1nc(Nc2ccc(N3CCNCC3)cc2)ncc1C(=O)Nc1cc(C(=O)Nc2cccc(C(F)(F)F)c2F)ccc1C]

As defined generally above and discussed throughout, R¹ is selected from an optionally substituted C₁-C₆ alkyl and an optionally substituted C₃-C₆ cycloalkyl.

In some embodiments, R¹ is optionally substituted C₁-C₆ alkyl. In some embodiments, R¹ is C₁-C₆ alkyl. In some embodiments, R¹ is selected from methyl, ethyl, propyl, isopropyl, n-butyl, and t-butyl. In some embodiments, R¹ is methyl. In some embodiments, R¹ is ethyl. In some embodiments, R¹ is propyl. In some embodiments, R¹ is isopropyl. In some embodiments, R¹ is n-butyl. In some embodiments, R¹ is t-butyl.

In some embodiments, R¹ is C₁-C₆ alkyl optionally substituted with a group selected from halogen, —(CH₂)₀₋₄R^{∘}, —(CH₂)₀₋₄OR^{∘}, and —(CH₂)₀₋₄NR^{∘}₂. In some embodiments, R¹ is C₁-C₄ alkyl optionally substituted with a group selected from halogen, —(CH₂)₀₋₄R^{∘}, —(CH₂)₀₋₄OR^{∘}, and —(CH₂)₀₋₄NR^{∘}₂. In some embodiments, R¹ is C₁-C₂ alkyl optionally substituted with a group selected from halogen, —(CH₂)₀₋₄R^{∘}, —(CH₂)₀₋₄OR^{∘}, and —(CH₂)₀₋₄NR^{∘}₂.

In some embodiments, R¹ is optionally substituted methyl. In some embodiments, R¹ is methyl optionally substituted with one or more —(CH₂)₀₋₄R^{∘}. In some embodiments, R¹ is methyl optionally substituted with one or more R^{∘}. In some embodiments, R¹ is methyl optionally substituted with one R^{∘}. In some such embodiments, R^{∘} is a 5-6-membered saturated, partially unsaturated, or aryl ring having 0-4 heteroatoms independently selected from nitrogen, oxygen, and sulfur. In some embodiments, R^{∘} is a 5-membered saturated ring having 0-2 heteroatoms independently selected from nitrogen, oxygen, and sulfur. In some embodiments, R^{∘} is a 5-membered saturated ring having 1 heteroatom selected from nitrogen, oxygen, and sulfur. In some embodiments, R^{∘} is a tetrahydrofuranyl. In some embodiments, R¹ is tetrahydrofuran-2-ylmethyl.

In some embodiments, R¹ is optionally substituted ethyl. In some embodiments, R¹ is ethyl optionally substituted with one or more groups selected from halogen and —(CH₂)₀₋₄OR^{°}.

In some embodiments, R¹ is ethyl optionally substituted with one to three halogen. In some embodiments, R¹ is ethyl optionally substituted with three halogen. In some embodiments, halogen is selected from bromo and iodo. In some embodiments, halogen is bromo. In some embodiments, halogen is iodo. In some embodiments, halogen is selected from chloro and fluoro. In some embodiments, halogen is chloro. In some embodiments, halogen is fluoro. In some such embodiments, R¹ is ethyl optionally substituted with three fluoro. In some embodiments, R¹ is 2,2,2-trifluoroethyl.

In some embodiments, R¹ is ethyl optionally substituted with one or more —(CH₂)₀₋₄OR^{∘}. In some embodiments, R¹ is ethyl substituted with one to two —(CH₂)₀₋₄OR^{∘}. In some embodiments, R¹ is ethyl substituted with one to two —OR^{∘}. In some such embodiments, R^{∘} is hydrogen. In some embodiments, R¹ is 2-hydroxyethyl.

In some embodiments, R¹ is optionally substituted propyl. In some embodiments, R¹ is propyl. In some embodiments, R¹ is propyl optionally substituted with one or more groups selected from —(CH₂)₀₋₄OR^{∘} and (CH₂)₀₋₄N(R^{∘})₂.

In some embodiments, R¹ is propyl optionally substituted with one or more —(CH₂)₀₋₄OR^{∘}. In some embodiments, R¹ is propyl substituted with one to three —(CH₂)₀₋₄OR^{∘}. In some embodiments, R¹ is propyl substituted with one to three —OR^{∘}. In some such embodiments, R^{∘} is hydrogen. In some embodiments, R¹ is 3-hydroxypropyl.

In some embodiments, R¹ is propyl optionally substituted with one or more (CH₂)₀₋₄N(R^{∘})₂. In some embodiments, R¹ is propyl substituted with one to three (CH₂)₀₋₄N(R^{∘})₂. In some embodiments, R¹ is propyl substituted with one to three N(R^{∘})₂. In some such embodiments, R^{∘} is C₁₋₆ aliphatic. In some embodiments, R^{∘} is C₁₋₄ aliphatic. In some embodiments, R^{∘} is methyl. In some embodiments, R¹ is 3-dimethylaminopropyl.

In some embodiments, R¹ is optionally substituted C₃-C₆ cycloalkyl. In some embodiments, R¹ is C₃-C₆ cycloalkyl. In some embodiments, R¹ is optionally substituted C₃-C₅ cycloalkyl. In some embodiments, R¹ is optionally substituted cyclopropyl. In some embodiments, R¹ is cyclopropyl.

In some embodiments, R¹ is selected from methyl, 2,2,2-trifluoroethyl, 2-hydroxyethyl, cyclopropyl, 3-hydroxypropyl, 3-dimethylaminopropyl, and tetrahydrofuran-2-ylmethyl.

As defined generally above and discussed throughout, R² is selected from an optionally substituted aryl and an optionally substituted heteroaryl, wherein two substituents on the aryl or heteroaryl are optionally taken together to form a saturated ring fused to the aryl or heteroaryl. The resulting saturated ring formed by taking the two substituents together may be a cycloalkyl or a heterocyclyl ring.

In some embodiments, R² is optionally substituted aryl. In some embodiments, R² is aryl. In some embodiments, R² is optionally substituted phenyl. In some embodiments, R² is phenyl. In some embodiments, R² is phenyl optionally substituted with one or more groups selected from —(CH₂)₀₋₄R^{∘}, —(CH₂)₀₋₄OR^{∘}, —CN, —(CH₂)₀₋₄C(O)R^{∘}, and a 5-7-membered saturated, partially unsaturated, or aryl ring having 0-4 heteroatoms independently selected from nitrogen, oxygen, and sulfur. In some embodiments, R² is phenyl optionally substituted with one or more groups selected from halo, —(CH₂)₀₋₄R^{∘}, —(CH₂)₀₋₄OR^{∘}, —CN, —(CH₂)₀₋₄C(O)R^{∘}, and a 5-7-membered saturated, partially unsaturated, or aryl ring having 0-4 heteroatoms independently selected from nitrogen, oxygen, and sulfur.

In some embodiments, R² is phenyl optionally substituted with halo. In some embodiments, R² is phenyl substituted with chloro. In some embodiments, R² is phenyl substituted with fluoro.

In some embodiments, R² is phenyl optionally substituted with one or more —(CH₂)₀₋₄R^{∘}. In some embodiments, R² is phenyl optionally substituted with one or more —R^{∘}. In some embodiments, —R^{∘} is C_{1 6} aliphatic. In some embodiments, —R^{∘} is C_{1 3} aliphatic. In some such embodiments, —R^{∘} is methyl.

In some embodiments, R² is phenyl optionally substituted with one or more —(CH₂)₀₋₄OR^{∘}. In some embodiments, R² is phenyl optionally substituted with one or more —OR^{∘}. In some such embodiments, R^{∘} is selected from C₁₋₆ aliphatic and a 5-6-membered saturated, partially unsaturated, or aryl ring having 0-4 heteroatoms independently selected from nitrogen, oxygen, and sulfur.

In some embodiments R² is phenyl optionally substituted with —OR^{∘}, wherein R^{∘} is C₁₋₆ aliphatic. In some embodiments R² is phenyl substituted with methoxy. In some embodiments R² is phenyl substituted with ethoxy. In some embodiments R² is phenyl substituted with propoxy.

In some embodiments R² is phenyl optionally substituted with —OR^{∘}, wherein R^{∘} is C₁₋₆ aliphatic optionally substituted with a group selected from —(CH₂)₀₋₂NR^{●}₂ and —(CH₂)₀₋₂R^{●}. In some embodiments, R^{∘} is C₁₋₃ aliphatic optionally substituted with a group selected from —NR^{●}₂ and —R^{●}. In some embodiments, R^{●} is selected from C₁₋₄ aliphatic and a 5-6-membered saturated, partially unsaturated, or aryl ring having 0-4 heteroatoms independently selected from nitrogen, oxygen, and sulfur. In some embodiments, R^{●} is C₁₋₄ aliphatic. In some embodiments, R^{●} is C₁₋₂ aliphatic. In some embodiments, R^{●} is methyl. In some embodiments, R^{●} is a 5-6-membered saturated, partially unsaturated, or aryl ring having 0-4 heteroatoms independently selected from nitrogen, oxygen, and sulfur. In some embodiments, R^{●} is a 5-membered saturated ring having 0-2 heteroatoms independently selected from nitrogen and oxygen. In some embodiments, R^{●} is a 6-membered saturated ring having 0-2 heteroatoms independently selected from nitrogen and oxygen. In some embodiments, R^{●} is morpholinyl.

In some embodiments R² is phenyl optionally substituted with —OR^{∘}, wherein R^{∘} is an optionally substituted 5-6-membered saturated, partially unsaturated, or aryl ring having 0-4 heteroatoms independently selected from nitrogen, oxygen, and sulfur. In some embodiments, R^{∘} is an optionally substituted 5-membered saturated ring having 1-2 nitrogen atoms. In some embodiments, R^{∘} is a 5-membered saturated ring having 1-2 nitrogen atoms. In some embodiments, R^{∘} is optionally substituted with —R^{●}. In some embodiments, —R^{●} is C₁₋₄ aliphatic. In some such embodiments, —R^{●} is methyl. In some embodiments, R^{∘} is pyrrolidinyl. In some embodiments, R^{∘} is pyrrolidinyl substituted with methyl.

In some embodiments R² is phenyl optionally substituted with —OR^{∘}, wherein R^{∘} is an optionally substituted 5- to 6-membered saturated ring having 1-2 nitrogen atoms. In some embodiments, R^{∘} is an optionally substituted 6-membered saturated ring having 1-2 nitrogen atoms. In some embodiments, R^{∘} is an optionally substituted 5-membered saturated ring having 1 nitrogen atoms. In some embodiments, R^{∘} is optionally substituted with —R^{†}. In some embodiments, —R^{†} is C₁₋₆ aliphatic. In some embodiments, —R^{†} is C₁₋₃ aliphatic. In some such embodiments, —R^{†} is methyl. In some embodiments, R^{∘} is piperidinyl. In some embodiments, R^{∘} is piperidinyl substituted with methyl. In some embodiments, R^{∘} is pyrrolidinyl. In some embodiments, R^{∘} is pyrrolidinyl substituted with methyl.

In some embodiments, R² is phenyl optionally substituted with —CN.

In some embodiments, R² is phenyl optionally substituted with one or more —(CH₂)₀₋₄C(O)R^{∘}. In some embodiments, R² is phenyl optionally substituted with one or more —C(O)R^{∘}. In some embodiments R² is phenyl optionally substituted with —C(O)R^{∘}, wherein R^{∘} is an optionally substituted 5-6-membered saturated, partially unsaturated, or aryl ring having 0-4 heteroatoms independently selected from nitrogen, oxygen, and sulfur. In some embodiments, R^{∘} is an optionally substituted 5-membered saturated ring having 0-2 nitrogen atoms. In some embodiments, R^{∘} is an optionally substituted 6-membered saturated ring having 0-2 nitrogen atoms. In some embodiments, R^{∘} is substituted with —(CH₂)₀₋₂R*. In some embodiments, R^{∘} is substituted with —R^{●}. In some embodiments, —R^{●} is C₁₋₄ aliphatic. In some embodiments, R^{∘} is piperazinyl. In some embodiments, R^{∘} is piperazinyl substituted with methyl.

In some embodiments, R² is phenyl optionally substituted with a 5-7-membered saturated, partially unsaturated, or aryl ring having 0-4 heteroatoms independently selected from nitrogen, oxygen, and sulfur.

In some embodiments, R² is phenyl optionally substituted with a 5-membered saturated ring having 0-2 nitrogen atoms. In some embodiments, R² is phenyl substituted with pyrrolidinyl, wherein the pyrrolidinyl is optionally substituted. In some embodiments, R² is phenyl substituted with pyrrolidinyl, wherein the pyrrolidinyl is substituted with methyl. In some embodiments, R² is phenyl substituted with pyrrolidinyl, wherein a saturated carbon atom in the pyrrolidinyl ring is divalently substituted with a 4-7 membered saturated, partially unsaturated, or aryl ring having 0-4 heteroatoms independently selected from nitrogen, oxygen, and sulfur. In some embodiments, R² is phenyl substituted with diazaspiro[5.5]undecyl, wherein the diazaspiro[5.5]undecyl is optionally substituted. In some embodiments, R² is phenyl substituted with diazaspiro[5.5]undecyl, wherein the diazaspiro[5.5]undecyl is substituted with methyl.

In some embodiments, R² is phenyl optionally substituted with a 6-membered saturated ring having 0-2 nitrogen atoms. In some such embodiments, a 6-membered saturated ring having 0-2 nitrogen atoms is optionally substituted with a group selected from —(CH₂)₀₋₂R^{●} and —CN. In some embodiments, a 6-membered saturated ring having 0-2 nitrogen atoms is optionally substituted with a group selected from —R^{●} and —CN. In some embodiments, R^{●} is C₁₋₄ aliphatic. In some embodiments, —R^{●} is C₁₋₃ aliphatic. In some such embodiments, —R^{●} is methyl. In some embodiments, R² is phenyl optionally substituted with piperidinyl. In some embodiments, R² is phenyl optionally substituted with piperidinyl substituted with a group selected from methyl and cyano. In some embodiments, R² is phenyl optionally substituted with piperidinyl substituted with methyl. In some embodiments, R² is phenyl optionally substituted with piperidinyl substituted with —CN. In some embodiments, R² is phenyl optionally substituted with piperazinyl. In some embodiments, R² is phenyl optionally substituted with piperazinyl substituted with methyl. In other such embodiments, R² is phenyl optionally substituted with a 6-membered saturated ring having 0-2 nitrogen atoms, wherein the 6-membered saturated ring having 0-2 nitrogen atoms is substituted with —N(C₁-C₄ alkyl)₂.

In some embodiments, R² is phenyl optionally substituted with a 7-membered saturated ring having 0-2 nitrogen atoms. In some such embodiments, the 7-membered saturated ring having 0-2 nitrogen atoms is optionally substituted. In some embodiments, R² is phenyl substituted with diazepanyl. In some embodiments, R² is phenyl substituted with diazepanyl, wherein the diazepanyl is substituted with oxo. In some embodiments, R² is phenyl substituted with diazepanyl, wherein the diazepanyl is substituted with methyl. In some embodiments, R² is phenyl substituted with diazepanyl, wherein the diazepanyl is substituted with methyl and oxo. In some embodiments, R² is phenyl substituted with azepanyl. In some embodiments, R² is phenyl substituted with azepanyl, wherein the azepanyl is substituted with methyl.

In some embodiments, R² is phenyl optionally substituted with one or more groups selected from —CN, —C₁-C₆ alkyl, —C₁-C₆ haloalkyl, —O—C₁-C₆ alkylene-N(C₁-C₄ alkyl)₂, heterocyclyl, —O-heterocyclyl, —O—C₁-C₆ alkylene-heterocyclyl, —C₁-C₆ alkylene-heterocyclyl, and —C(O)-heterocyclyl, wherein each heterocyclyl portion of a substituent is optionally substituted with up to 3 substituents independently selected from oxo, —CN, and C₁-C₄ alkyl.

In some embodiments, R² is phenyl optionally substituted with one or more substituents independently selected from —CN, methyl, 1-methyl-4-cyanopiperidin-1-yl, 1-methylpiperidin-4-yl, 1-methylpiperidin-4-yloxy, 1-methylpyrrolidin-3-yl, 1-methylpyrrolidin-3-yloxy, 1-methylpyrrolidin-3-ylmethyl, 2-(morpholin-4-yl)ethan-1-oxy, 2-dimethylaminoethan-1-oxy, 2-dimethylaminoethan-1-yl, 3-(morpholin-4-yl)propan-1-oxy, 3-dimethylaminopropan-1-oxy, 7-oxo-1,4-diazepan-1-yl, 4-methylpiperazin-1-yl, 4-methylpiperazin-1-ylcarbonyl, piperazin-1-yl, piperidin-4-yl, piperidin-4-yloxy, pyrrolidin-2-ylmethyl, pyrrolidin-3-ylmethyl, and pyrrolidin-3-yloxy.

In some embodiments, R² is phenyl optionally substituted with one or more substituents independently selected from CN, chloro, fluoro, methyl, methoxy, ethoxy, propoxy, 1,4-diazepan-1-yl, 1-methyl-4-cyanopiperidin-1-yl, 1-methylazepan-4-yl, 1-methylpiperidin-3-yl, 1-methylpiperidin-4-yl, 1-methylpiperidin-4-yloxy, 1-methylpyrrolidin-3-yl, 1-methylpyrrolidin-3-ylmethyl, 1-methylpyrrolidin-3-yloxy, 1-methylpyrrol-3-yl, 2-(morpholin-4-yl)ethan-1-oxy, 2-dimethylaminoethan-1-oxy, 2-dimethylaminoethan-1-yl, 3-(morpholin-4-yl)propan-1-oxy, 3-dimethylaminopropan-1-oxy, 4-dimethylaminopiperidin-1-yl, 4-methylpiperazin-1-yl, 4-methylpiperazin-1-ylcarbonyl, 4-methyl-7-oxo-1,4-diazepan-1-yl, 4-methyl-1,4-diazepan-1-yl, 7-oxo-1,4-diazepan-1-yl, 9-methyl-3,9-diazaspiro[5.5]undec-3-yl, pyrrolidin-3-yloxy, piperazin-1-yl, piperidin-3-yl, piperidin-4-yl, piperidin-4-yloxy, pyrrolidin-2-ylmethyl, pyrrolidin-3-ylmethyl, and pyrrolidin-3-yl.

In some embodiments, R² is phenyl having 1 to 3 substituents independently selected from those set forth above.

In some embodiments, R² is selected from:

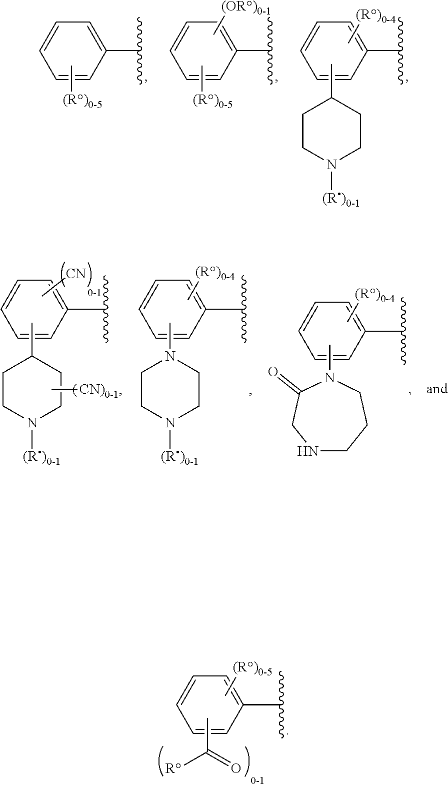

In some embodiments, R² is selected from:

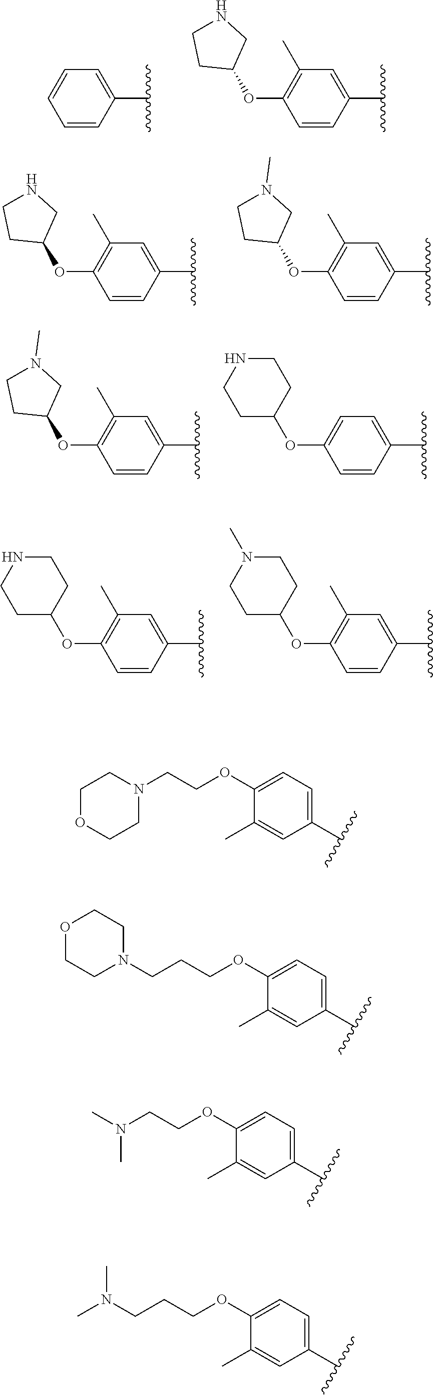

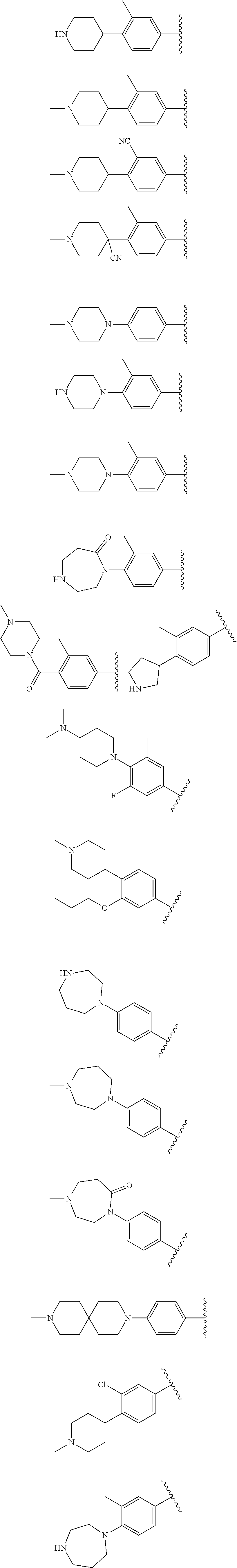

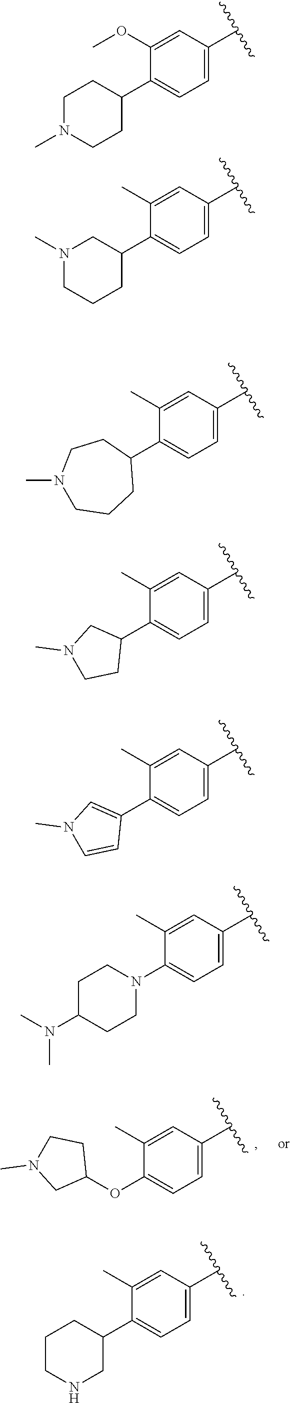

In some embodiments, R² is optionally substituted aryl wherein two substituents on the aryl are optionally taken together to form a saturated ring fused to the aryl.

In some embodiments, R² is optionally substituted 1,2,3,4-tetrahydroisoquinolinyl. In some embodiments, R² is 1,2,3,4-tetrahydroisoquinolinyl. In some embodiments, R² is 1,2,3,4-tetrahydroisoquinolinyl optionally substituted one or more groups selected from —(CH₂)₀₋₄R^{∘} and —R^{†}. In some such embodiments, R² is 1,2,3,4-tetrahydroisoquinolinyl optionally substituted with one to three —(CH₂)₁₋₂R^{∘} and —R^{†}. In some embodiments, R^{∘} is CH₃. In some embodiments, R² is 1,2,3,4-tetrahydroisoquinolinyl optionally substituted only with —R^{†}. In some embodiments, —R^{†} is C₁₋₆ aliphatic. In some embodiments, —R^{†} is C₁₋₃ aliphatic. In some such embodiments, —R^{†} is methyl.

In some embodiments, R² is 1,2,3,4-tetrahydroisoquinolinyl optionally substituted with one —(CH₃). In some embodiments, R² is 1,2,3,4-tetrahydroisoquinolinyl optionally substituted with three —(CH₃).

In some embodiments, R² is 1,2,3,4-tetrahydroisoquinolinyl optionally substituted with one or more groups selected from —CN, —C₁-C₆ alkyl, —C₁-C₆ haloalkyl, —O—C₁-C₆ alkylene-N(C₁-C₄ alkyl)₂, heterocyclyl, —O-heterocyclyl, —O—C₁-C₆ alkylene-heterocyclyl, —C₁-C₆ alkylene-heterocyclyl, and —C(O)-heterocyclyl, wherein each heterocyclyl portion of a substituent is optionally substituted with up to 3 substituents independently selected from oxo, —CN, and C₁-C₄ alkyl.

In some embodiments, R² is 1,2,3,4-tetrahydroisoquinolinyl optionally substituted with one or more substituents independently selected from —CN, methyl, 1-methyl-4-cyanopiperidin-1-yl, 1-methylpiperidin-4-yl, 1-methylpiperidin-4-yloxy, 1-methylpyrrolidin-3-yl, 1-methylpyrrolidin-3-yloxy, 1-methylpyrrolidin-3-ylmethyl, 2-(morpholin-4-yl)ethan-1-oxy, 2-dimethylaminoethan-1-oxy, 2-dimethylaminoethan-1-yl, 3-(morpholin-4-yl)propan-1-oxy, 3-dimethylaminopropan-1-oxy, 7-oxo-1,4-diazepan-1-yl, 4-methylpiperazin-1-yl, 4-methylpiperazin-1-ylcarbonyl, piperazin-1-yl, piperidin-4-yl, piperidin-4-yloxy, pyrrolidin-2-ylmethyl, pyrrolidin-3-ylmethyl, and pyrrolidin-3-yloxy.

In some embodiments, R² is 1,2,3,4-tetrahydroisoquinolinyl having 1 to 3 substituents independently selected from those set forth above.

In some embodiments, R² is phenyl optionally substituted with two independent occurrences of R^{∘}, wherein the two occurrences of R^{∘} are taken together with their intervening atom(s), to form a 3-12-membered saturated mono- or bicyclic ring having 0-4 heteroatoms independently selected from nitrogen, oxygen, and sulfur, which may be substituted, thus resulting in R² being a bicyclic (when two independent occurrences of R^{∘} are taken together to form a saturated monocyclic ring) or tricyclic (when two independent occurrences of R^{∘} are taken together to form a saturated bicyclic ring) ring system. In some embodiments, R² is phenyl optionally substituted with two independent occurrences of R^{∘} taken together with their intervening atom(s), to form a 5-7-membered saturated monocyclic ring having 0-2 heteroatoms independently selected from nitrogen and oxygen, which may be substituted. In some embodiments, R² is phenyl optionally substituted with two independent occurrences of R^{∘} taken together with their intervening atom(s), to form a 6-membered saturated monocyclic ring having one nitrogen, which may be substituted with one or more methyl groups. In some embodiments, two independent occurrences of R^{∘} taken together with their intervening atoms form piperidinyl, resulting in R² being tetrahydroisoquinolinyl. In some embodiments, two independent occurrences of R^{∘} taken together with their intervening atoms form piperidinyl, resulting in R² being 1,2,3,4-tetrahydroisoquinolin-7-yl optionally substituted with 1 to 3 methyl groups.

In some embodiments, R² is

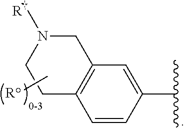

In some embodiments, R² is selected from

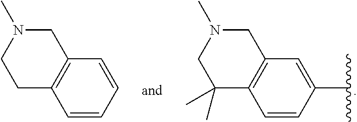

In some embodiments, R² is optionally substituted heteroaryl. In some embodiments, R² is heteroaryl. In some embodiments, R² is optionally substituted 5- and 6-membered heteroaryl having 1-4 heteroatoms independently selected from nitrogen, oxygen, and sulfur.

In some embodiments, R² is optionally substituted 5-membered heteroaryl having 1-2 heteroatoms independently selected from nitrogen, oxygen, and sulfur. In some embodiments, R² is optionally substituted 5-membered heteroaryl having 2 nitrogen atoms. In some embodiments, R² is optionally substituted 1H-pyrazolyl. In some embodiments, R² is 1H-pyrazolyl. In some embodiments, R² is 1H-pyrazolyl optionally substituted with R. In some embodiments, R² is 1H-pyrazolyl substituted with a 7-membered saturated heterocyclic ring having 1-2 nitrogen atoms. In some such embodiments, —R^{†} is selected from C₁₋₆ aliphatic and a 5-6-membered saturated, partially unsaturated, or aryl ring having 0-4 heteroatoms independently selected from nitrogen, oxygen, and sulfur. In some embodiments, R^{†} is C₁₋₄ aliphatic. In some embodiments, —R^{†} is C₁₋₃ aliphatic. In some such embodiments, —R^{†} is methyl.

In some embodiments, —R^{†} is C₁₋₃ aliphatic substituted with —R^{●}. In some such embodiments, —R^{●} is a 5-6-membered saturated, partially unsaturated, or aryl ring having 0-4 heteroatoms independently selected from nitrogen, oxygen, and sulfur. In some embodiments, —R^{●} is a 5-membered saturated ring having 1 heteroatom selected from nitrogen and oxygen. In some embodiments, —R^{●} is pyrrolidinyl. In some embodiments, —R^{●} is —NH(CH₃). In some embodiments, —R^{●} is —N(CH₃)₂.

In some embodiments, —R^{†} is a 5-6-membered saturated, partially unsaturated, or aryl ring having 0-4 heteroatoms independently selected from nitrogen, oxygen, and sulfur. In some embodiments, —R^{†} is a 6-membered saturated ring having 1-2 heteroatoms independently selected from nitrogen and oxygen. In some embodiments, —R^{†} is piperidinyl. In some embodiments, —R^{†} is piperidinyl substituted with —R^{●}. In some such embodiments, —R^{●} is C₁₋₄ aliphatic. In some embodiments, —R^{●} is C₁₋₃ aliphatic. In some such embodiments, —R^{●} is methyl.

In some embodiments, R² is 1H-pyrazolyl substituted with a 7-membered saturated heterocyclic ring comprising 1-2 nitrogen atoms. In some embodiments, R² is 1H-pyrazolyl substituted with diazepanyl, wherein the diazepanyl is optionally substituted. In some embodiments, R² is 1H-pyrazolyl substituted with diazepanyl, wherein the diazepanyl is substituted with methyl. In some embodiments, R² is 1H-pyrazolyl substituted with azepanyl, wherein the azepanyl is optionally substituted. In some embodiments, R² is 1H-pyrazolyl substituted with azepanyl, wherein the azepanyl is substituted with methyl.

In some embodiments, R² is 1H-pyrazolyl optionally substituted with one or more groups selected from —CN, —C₁-C₆ alkyl, —C₁-C₆ haloalkyl, —O—C₁-C₆ alkylene-N(C₁-C₄ alkyl)₂, heterocyclyl, —O-heterocyclyl, —O—C₁-C₆ alkylene-heterocyclyl, —C₁-C₆ alkylene-heterocyclyl, and —C(O)-heterocyclyl, wherein each heterocyclyl portion of a substituent is optionally substituted with up to 3 substituents independently selected from oxo, —CN, and C₁-C₄ alkyl.

In some embodiments, R² is 1H-pyrazolyl substituted with —C₁-C₆ alkylene-N(C₁-C₄ alkyl)₂ or —C₁-C₆ alkylene-NH(C₁-C₄ alkyl).

In some embodiments, R² is 1H-pyrazolyl optionally substituted with one or more substituents independently selected from —CN, methyl, 1-methyl-4-cyanopiperidin-1-yl, 1-methylpiperidin-4-yl, 1-methylpiperidin-4-yloxy, 1-methylpyrrolidin-3-yl, 1-methylpyrrolidin-3-yloxy, 1-methylpyrrolidin-3-ylmethyl, 2-(morpholin-4-yl)ethan-1-oxy, 2-dimethylaminoethan-1-oxy, 2-dimethylaminoethan-1-yl, 3-(morpholin-4-yl)propan-1-oxy, 3-dimethylaminopropan-1-oxy, 7-oxo-1,4-diazepan-1-yl, 4-methylpiperazin-1-yl, 4-methylpiperazin-1-ylcarbonyl, piperazin-1-yl, piperidin-4-yl, piperidin-4-yloxy, pyrrolidin-2-ylmethyl, pyrrolidin-3-ylmethyl, and pyrrolidin-3-yloxy.

In some embodiments, R² is 1H-pyrazolyl substituted with 1-methyl-2-dimethylaminoethan-1-yl, 1-methyl-2-methylaminoethan-1-yl, 3-dimethylaminopropan-1-yl, azepan-4-yl, or 1-methylazepan-4-yl.

In some embodiments, R² is 1H-pyrazolyl having 1 to 3 substituents independently selected from those set forth above.

In some embodiments, R² is

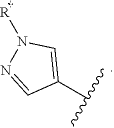

In some embodiments, R² is selected from

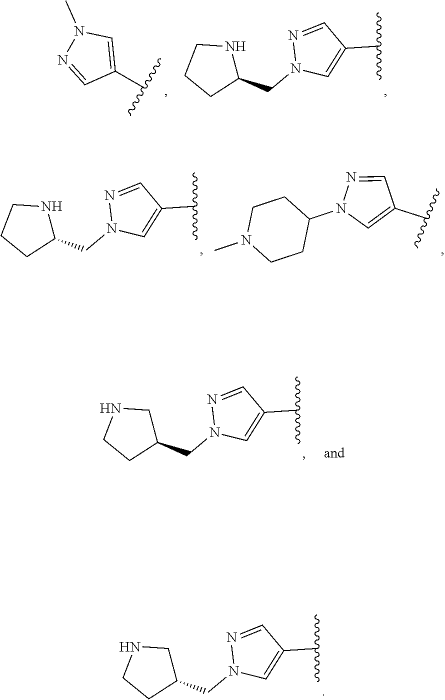

In some embodiments R² is selected from

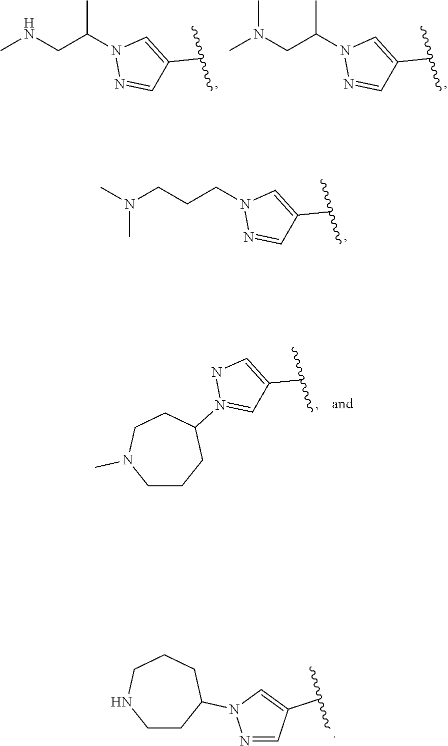

In some embodiments, R² is optionally substituted 6-membered heteroaryl having 1-2 heteroatoms independently selected from nitrogen, oxygen, and sulfur. In some embodiments, R² is optionally substituted 6-membered heteroaryl having 2 nitrogen atoms. In some embodiments, R² is optionally substituted pyridinyl. In some embodiments, R² is pyridinyl. In some embodiments, R² is pyridinyl optionally substituted with one or more groups selected from —(CH₂)₀₋₄R^{∘} and —(CH₂)₀₋₄OR^{∘}. In some embodiments, R² is pyridinyl optionally substituted with one or more groups selected from —R^{∘} and —OR^{∘}. In some such embodiments, R^{∘} is selected from C₁₋₆ aliphatic and a 5-6-membered saturated, partially unsaturated, or aryl ring having 0-4 heteroatoms independently selected from nitrogen, oxygen, and sulfur.

In some embodiments, R² is pyridinyl optionally substituted with R^{∘}, wherein R^{∘} is C₁₋₆ aliphatic. In some embodiments, R² is pyridinyl optionally substituted with R^{∘}, wherein R^{∘} is C₁₋₃ aliphatic. In some embodiments, R² is pyridinyl optionally substituted with R^{∘}, wherein R^{∘} is methyl.

In some embodiments, R² is pyridinyl optionally substituted with —OR^{∘}, wherein R^{∘} is a 5-6-membered saturated, partially unsaturated, or aryl ring having 0-4 heteroatoms independently selected from nitrogen, oxygen, and sulfur. In some embodiments, R² is pyridinyl optionally substituted with R^{∘}, wherein R^{∘} is a 6-membered saturated ring having 1-2 nitrogen, wherein R^{∘} is optionally substituted with —R^{●}. In some embodiments, R^{∘} is piperidinyl. In some such embodiments, —R^{●} is C₁₋₄ aliphatic. In some such embodiments, —R^{●} is methyl.

In some embodiments, R² is pyridinyl optionally substituted with one or more groups selected from —CN, —C₁-C₆ alkyl, —C₁-C₆ haloalkyl, —O—C₁-C₆ alkylene-N(C₁-C₄ alkyl)₂, heterocyclyl, —O-heterocyclyl, —O—C₁-C₆ alkylene-heterocyclyl, —C₁-C₆ alkylene-heterocyclyl, and —C(O)-heterocyclyl, wherein each heterocyclyl portion of a substituent is optionally substituted with up to 3 substituents independently selected from oxo, —CN, and C₁-C₄ alkyl.

In some embodiments, R² is pyridinyl optionally substituted with one or more substituents independently selected from —CN, methyl, 1-methyl-4-cyanopiperidin-1-yl, 1-methylpiperidin-4-yl, 1-methylpiperidin-4-yloxy, 1-methylpyrrolidin-3-yl, 1-methylpyrrolidin-3-yloxy, 1-methylpyrrolidin-3-ylmethyl, 2-(morpholin-4-yl)ethan-1-oxy, 2-dimethylaminoethan-1-oxy, 2-dimethylaminoethan-1-yl, 3-(morpholin-4-yl)propan-1-oxy, 3-dimethylaminopropan-1-oxy, 7-oxo-1,4-diazepan-1-yl, 4-methylpiperazin-1-yl, 4-methylpiperazin-1-ylcarbonyl, piperazin-1-yl, piperidin-4-yl, piperidin-4-yloxy, pyrrolidin-2-ylmethyl, pyrrolidin-3-ylmethyl, and pyrrolidin-3-yloxy.

In some embodiments, R² is pyridinyl optionally substituted with one or more substituents independently selected from 1-methylpiperidin-3-yl, 1-methylpiperidin-4-yl, 1-methylpiperidin-4-yloxy, 1-methylpyrrolidin-3-yl, 1-methylpyrrolidin-3-yloxy, I-methylpyrrolidin-3-ylmethyl, 2-(morpholin-4-yl)ethan-1-oxy, 2-dimethylaminoethan-1-oxy, 2-dimethylaminoethan-1-yl, 3-(morpholin-4-yl)propan-1-oxy, 3-dimethylaminopropan-1-oxy, 7-oxo-1,4-diazepan-1-yl, 4-methylpiperazin-1-yl, 4-methylpiperazin-1-ylcarbonyl, piperazin-1-yl, piperidin-4-yl, piperidin-4-yloxy, pyrrolidin-2-ylmethyl, pyrrolidin-3-ylmethyl, and pyrrolidin-3-yloxy.

In some embodiments, R² is pyridinyl having 1 to 3 substituents independently selected from those set forth above.

In some embodiments, R² is

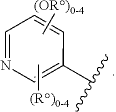

In some embodiments, R² is

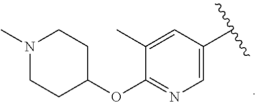 [SMILES: *c1cnc(OC2CCN(C)CC2)c(C)c1]

In some embodiments, R² is

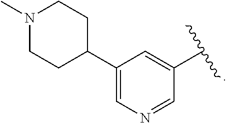 [SMILES: *c1cncc(C2CCN(C)CC2)c1]

In some embodiments, R² is optionally substituted heteroaryl wherein two substituents on the heteroaryl are optionally taken together to form a saturated ring fused to the heteroaryl.

In some embodiments, R² is optionally substituted 1H-indazolyl. In some embodiments, R² is 1H-indazolyl. In some embodiments, R² is 1H-indazolyl optionally substituted with —Rf. In some embodiments, —R— is selected from C₁₋₆ aliphatic and a 5-6-membered saturated, partially unsaturated, or aryl ring having 0-4 heteroatoms independently selected from nitrogen, oxygen, and sulfur.

In some embodiments, —R^{†} is C₁₋₆ aliphatic. In some embodiments, —R^{†} is C₁₋₃ aliphatic. In some embodiments, —R^{†} is methyl. In some embodiments, —R^{†} is C₁₋₂ aliphatic optionally substituted with —NR^{●}₂. In some embodiments, R^{●} is C₁₋₄ aliphatic. In some embodiments, R^{●} is C₁₋₂ aliphatic. In some embodiments, R^{●} is methyl. In some embodiments, —R^{†} is methyl.

In some embodiments, R^{†} is a 5-6-membered saturated, partially unsaturated, or aryl ring having 0-4 heteroatoms independently selected from nitrogen, oxygen, and sulfur. In some embodiments, R^{†} is a 5-membered saturated ring having 1-2 nitrogen atoms optionally substituted with R^{●}. In some embodiments, R^{†} is pyrrolidinyl. In some embodiments, R^{●} is C₁₋₄ aliphatic. In some embodiments, R^{●} is C₁₋₂ aliphatic. In some embodiments, R^{●} is methyl.

In some embodiments, R^{†} is a 6-membered saturated ring having 1-2 nitrogen atoms optionally substituted with R^{●}. In some embodiments, R^{†} is piperidinyl. In some embodiments, R^{●} is C₁₋₄ aliphatic. In some embodiments, R^{●} is C₁₋₂ aliphatic. In some embodiments, R^{●} is methyl.

In some embodiments, R² is 1H-indazolyl optionally substituted with one or more groups selected from —CN, —C₁-C₆ alkyl, —C₁-C₆ haloalkyl, —O—C₁-C₆ alkylene-N(C₁-C₄ alkyl)₂, heterocyclyl, —O-heterocyclyl, —O—C₁-C₆ alkylene-heterocyclyl, —C₁-C₆ alkylene-heterocyclyl, and —C(O)-heterocyclyl, wherein each heterocyclyl portion of a substituent is optionally substituted with up to 3 substituents independently selected from oxo, —CN, and —C₁-C₄ alkyl.

In some embodiments, R² is 1H-indazolyl optionally substituted with one or more substituents independently selected from —CN, methyl, 1-methyl-4-cyanopiperidin-1-yl, 1-methylpiperidin-4-yl, 1-methylpiperidin-4-yloxy, 1-methylpyrrolidin-3-yl, 1-methylpyrrolidin-3-yloxy, 1-methylpyrrolidin-3-ylmethyl, 2-(morpholin-4-yl)ethan-1-oxy, 2-dimethylaminoethan-1-oxy, 2-dimethylaminoethan-1-yl, 3-(morpholin-4-yl)propan-1-oxy, 3-dimethylaminopropan-1-oxy, 7-oxo-1,4-diazepan-1-yl, 4-methylpiperazin-1-yl, 4-methylpiperazin-1-ylcarbonyl, piperazin-1-yl, piperidin-4-yl, piperidin-4-yloxy, pyrrolidin-2-ylmethyl, pyrrolidin-3-ylmethyl, and pyrrolidin-3-yloxy.

In some embodiments, R² is 1H-indazolyl having 1 to 3 substituents independently selected from those set forth above.

In some embodiments, R² is

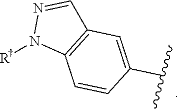

In some embodiments, R^{†} is C₁₋₆ aliphatic optionally substituted with —(CH₂)₀₋₂N(R^{●})₂ or —(CH₂)₀₋₂R^{●}. In some embodiments, R^{†} is C₁₋₆ aliphatic optionally substituted with —N(R^{●})₂ or —R^{●}. In some embodiments, R² is

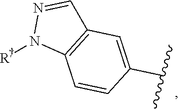

wherein R^{†} is C₁₋₆ aliphatic optionally substituted with —R^{●}, wherein —R^{●} is a 5- to 6-membered saturated, partially unsaturated, or aryl ring having 0-4 heteroatoms independently selected from nitrogen, oxygen, and sulfur.

In some embodiments, R² is selected from

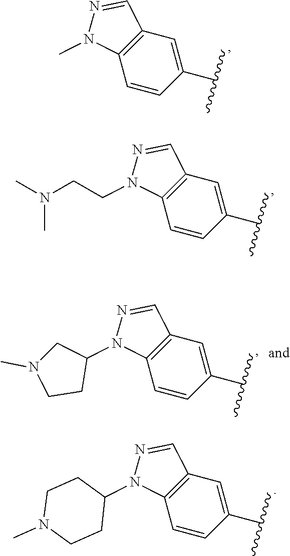 [SMILES: *c1ccc2c(cnn2C)c1 ; *c1ccc2c(cnn2CCN(C)C)c1 ; *c1ccc2c(cnn2C2CCN(C)C2)c1 ; *c1ccc2c(cnn2C2CCN(C)CC2)c1]

In some embodiments, R² is selected from R² is phenyl, pyridinyl, 1H-pyrazolyl, 1H-indazolyl, and 1,2,3,4-tetrahydroisoquinolinyl.

In some embodiments, R² is phenyl, 1-(1H-pyrrolidin-2-yl)methyl-1H-pyrazin-5-yl, 1-(1H-pyrrolidin-3-yl)methyl-1H-pyrazin-5-yl, 1-(1-methyl-1H-pyrrolidin-3-yl)-1H-indazol-5-yl, 1-(1-methylpiperidin-4-yl)-1H-indazol-5-yl, 1-(1-methylpiperidin-4-yl)pyrazin-5-yl, 1-(1-methylpyrrolidin-3-yl)methylpyrazin-4-yl, 1-(2-dimethylaminoethan-yl)-1H-indazol-5-yl, 1-methyl-1H-indazol-5-yl, 1-methyl-1H-pyrazin-5-yl, 2-(1-methylpiperidin-4-yl)oxy-3-methylpyridin-4-yl, 2,4,4-trimethyl-1,2,3,4-tetrahydroisoquinolin-7-yl, 2-methyl-1,2,3,4-tetrahydroisoquinolin-7-yl, 3-cyano-4-(1-methylpiperidin-4-yl)phenyl, 3-methyl-4-(1-methyl-1H-pyrrolidin-3-yl)oxyphenyl, 3-methyl-4-(1-methyl-4-cyanopiperidin-4-yl)phenyl, 3-methyl-4-(1-methylpiperidin-4-yl)oxyphenyl, 3-methyl-4-(1-methylpiperidin-4-yl)phenyl, 3-methyl-4-(2-dimethylaminoethan-1-yl)oxyphenyl, 3-methyl-4-(2-morpholin-4-ylethan-1-yl)oxyphenyl, 3-methyl-4-(3-dimethylaminopropan-1-yl)oxyphenyl, 3-methyl-4-(3-morpholin-4-ylpropan-1-yl)oxyphenyl, 3-methyl-4-(3-oxo-1,4-diazapin-1-yl)phenyl, 3-methyl-4-(4-methylpiperazin-1-yl)carbonylphenyl, 3-methyl-4-(4-methylpiperazin-1yl)phenyl, 3-methyl-4-piperazin-1-ylphenyl, 3-methyl-4-pyridin-1-ylphenyl, 3-methyl-4-pyridin-4-yloxyphenyl, 3-methyl-4-pyrrolidin-3-yloxyphenyl, 4-(4-methylpiperazin-1-yl)phenyl, or 4-piperidin-4-yloxyphenyl.

In some embodiments, R² is 1-(1-methyl-2,2-dimethylaminoethan-1-yl)pyrazol-4-yl, 1-(1-methyl-2-methylaminoethan-1-yl)pyrazol-4-yl, 1-(1-methylazepan-4-yl)pyrazol-4-yl, 1-(1-methylpiperidin-4-yl)pyrazol-4-yl, 1-azepan-4-ylpyrazol-4-yl, 1-methylpyrazol-4-yl, 3-chloro-4-(1-methylpiperidin-4-yl)phenyl, 3-ethoxy-4-(1-methylpiperidin-4-yl)phenyl, 3-methoxy-4-(1-methylpiperidin-4-yl)phenyl, 3-methyl-4-(1,4-diazepan-1-yl)phenyl, 3-methyl-4-(1-methylazepan-4-yl)phenyl, 3-methyl-4-(1-methylpiperidin-3-yl)phenyl, 3-methyl-4-(1-methylpyrrol-3-yl)phenyl, 3-methyl-4-(1-methylpyrrolidin-3-yl)phenyl, 3-methyl-4-(1-methyl-pyrrolidin-3-yloxy)phenyl, 3-methyl-4-(4-dimethylaminopiperidin-1-yl)phenyl, 3-methyl-4-(piperidin-4-yloxy)phenyl, 3-methyl-4-piperidin-3-ylphenyl, 3-methyl-4-piperidin-4-ylphenyl, 3-methyl-4-pyrrolidin-3-ylphenyl, 3-methyl-5-fluoro-4-(4-dimethylaminopiperidin-1-yl)phenyl, 3-propoxy-4-(1-methylpiperidin-4-yl)phenyl, 4-(1,4-diazepan-1-yl)phenyl, 4-(4-methyl-1,4-diazepan-1-yl)phenyl, 4-(4-methyl-7-oxo-1,4-diazepan-1-yl)phenyl, 4-(9-methyl-3,9-diazaspiro[5.5]undec-3-yl)phenyl, or 5-(1-methylpiperidin-4-yl)pyridin-3-yl.

As defined generally above and discussed throughout, R³ is an optionally substituted aryl or an optionally substituted heteroaryl other than tetrazolyl.

In some embodiments, R³ is optionally substituted aryl. In some embodiments, R³ is aryl. In some embodiments, R³ is optionally substituted phenyl. In some embodiments, R³ is phenyl. In some embodiments, R³ is phenyl optionally substituted with one or more groups selected from halogen, —CN, —NO₂, —(CH₂)₀₋₄R^{∘}, —(CH₂)₀₋₄OR^{∘}, (CH₂)₀₋₄N(R^{∘})₂, and —(CH₂)₀₋₄ N(R^{∘})C(O)R^{∘}. In some embodiments, R³ is phenyl optionally substituted with one or more halogen. In some embodiments, halogen is selected from fluoro and chloro. In some embodiments, halogen is fluoro. In some embodiments, halogen is chloro. In some embodiments, halogen is selected from bromo and iodo. In some embodiments, halogen is bromo. In some embodiments, halogen is iodo.

In some embodiments, R³ is phenyl optionally substituted with one or more —CN. In some embodiments, R³ is phenyl substituted with one to three —CN. In some embodiments, R³ is phenyl substituted with —CN.

In some embodiments, R³ is phenyl optionally substituted with one or more —NO₂. In some embodiments, R³ is phenyl substituted with one to three —NO₂. In some embodiments, R³ is phenyl substituted with —NO₂.

In some embodiments, R³ is phenyl optionally substituted with one or more —(CH₂)₀₋₄R^{∘}. In some embodiments, R³ is phenyl optionally substituted with one or more —R^{∘}. In some embodiments, R³ is phenyl optionally substituted with one to three —R^{∘}. In some embodiments, R^{∘} is C₁₋₆ aliphatic. In some embodiments, R^{∘} is C₁₋₃ aliphatic. In some embodiments, R^{∘} is ethyl. In some embodiments, R^{∘} is methyl.

In some embodiments, R³ is phenyl optionally substituted with one or more —(CH₂)₀₋₄OR^{∘}. In some embodiments, R³ is phenyl optionally substituted with one or more —OR^{∘}. In some embodiments, R³ is phenyl optionally substituted with one to three —OR^{∘}. In some embodiments, R^{∘} is C₁₋₆ aliphatic. In some embodiments, R^{∘} is C₁₋₃ aliphatic. In some embodiments, R^{∘} is methyl. In some embodiments, R^{∘} is hydrogen.

In some embodiments, R³ is phenyl optionally substituted with one or more (CH₂)₀₋₄N(R^{∘})₂. In some embodiments, R³ is phenyl optionally substituted with one or more N(R^{∘})₂. In some embodiments, R³ is phenyl optionally substituted with one to three N(R^{∘})₂. In some embodiments, R^{∘} is hydrogen.

In some embodiments, R³ is phenyl optionally substituted with one or more —(CH₂)₀₋₄N(R^{∘})C(O)R^{∘}. In some embodiments, R³ is phenyl optionally substituted with one or more —N(R^{∘})C(O)R^{∘}. In some embodiments, R³ is phenyl optionally substituted with one to three —N(R^{∘})C(O)R^{∘}. In some embodiments, R^{∘} is independently selected from C₁₋₆ aliphatic and hydrogen. In some such embodiments, R^{∘} is C₁₋₃ aliphatic. In some embodiments, R^{∘} is methyl. In some embodiments, R^{∘} is hydrogen.

In some embodiments, R³ is phenyl optionally substituted with 1 to 3 substituents independently selected from methyl, ethyl, fluoro, chloro, bromo, cyclopropyl, —OCH₃, —SCH₃, —NH₂, —NO₂, —CF₃, —CN, —C≡CH, —CH₂—CH═CH₂, —OH, and —NHC(O)CH₃.

In some embodiments, R³ has the structure:

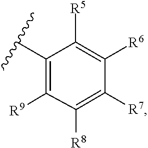

wherein:

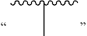

represents a point of attachment of R³ to the compound
R⁵ is hydrogen, chloro, bromo, or methyl;
R⁶ is hydrogen or fluoro;
R⁷ is hydrogen or fluoro;
R⁸ is hydrogen or —OH; and
R⁹ is hydrogen, chloro, fluoro, bromo, methyl, cyclopropyl, —C≡CH, —OCH₃, or —SCH₃, wherein at least one of R⁵ or R⁹ is other than hydrogen.

In some embodiments, R⁵ is hydrogen. In some embodiments, R⁵ is other than hydrogen.

In some embodiments, R⁵ is selected from chloro, bromo, and methyl. In some embodiments, R⁵ is chloro. In some embodiments, R⁵ is bromo. In some embodiments, R⁵ is methyl.

In some embodiments, Re is hydrogen. In some embodiments, Re is fluoro.

In some embodiments, R⁷ is hydrogen. In some embodiments, R⁷ is fluoro.

In some embodiments, R⁸ is hydrogen. In some embodiments, R⁸ is —OH.

In some embodiments, R⁹ is hydrogen. In some embodiments, R⁹ is other than hydrogen.

In some embodiments, R⁹ is selected from chloro, fluoro, bromo, methyl, cyclopropyl, —C≡CH, —OCH₃, and —SCH₃. In some embodiments, R⁹ is chloro. In some embodiments, R⁹ is fluoro. In some embodiments, R⁹ is bromo. In some embodiments, R⁹ is methyl. In some embodiments, R⁹ is cyclopropyl. In some embodiments, R⁹ is —C≡CH. In some embodiments, R⁹ is —OCH₃. In some embodiments, R⁹ is —SCH₃.

In some embodiments, R³ is phenyl having 1 to 3 substituents independently selected from those set forth above.

In some embodiment, R³ is selected from

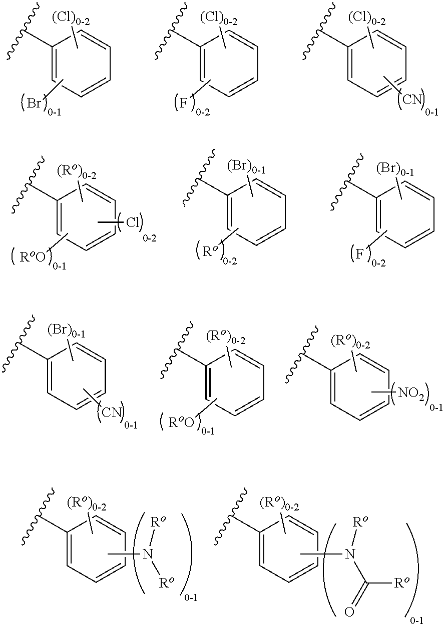

In some embodiments, R³ is selected from:

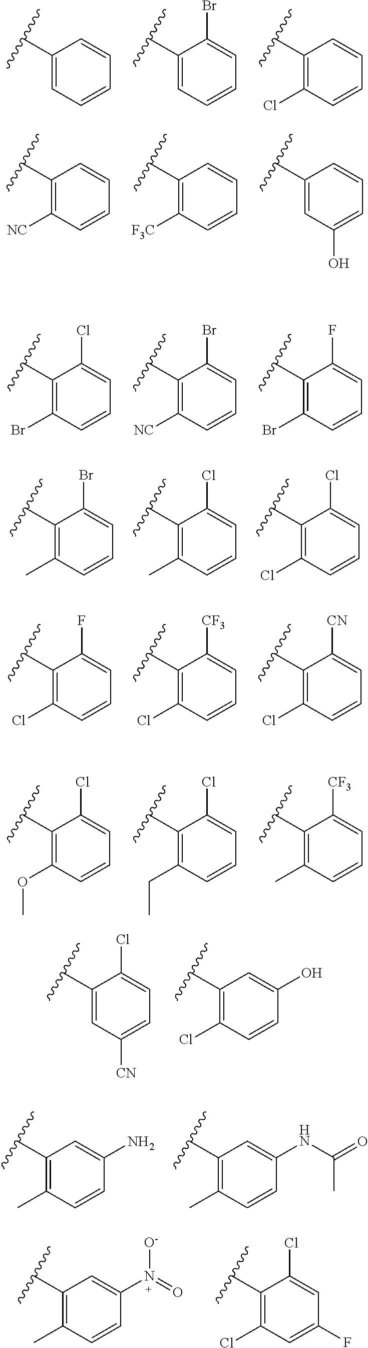

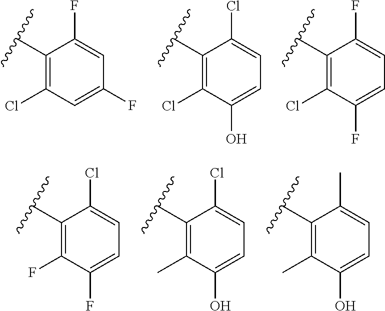

In some embodiments, R³ is selected from:

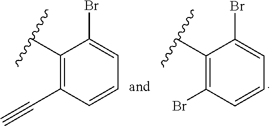

In some embodiments, R³ is optionally substituted aryl wherein two substituents on the aryl are optionally taken together to form a heterocyclic or heteroaryl ring fused to the aryl.

In some embodiments, R³ is optionally substituted heteroaryl other than tetrazolyl. In some embodiments, R³ is heteroaryl other than tetrazolyl. In some embodiments, R³ is optionally substituted 5- and 6-membered heteroaryl other than tetrazolyl having 1-4 heteroatoms independently selected from nitrogen, oxygen, and sulfur.

In some embodiments, R³ is optionally substituted 5-membered heteroaryl having 1-2 heteroatoms independently selected from nitrogen, oxygen, and sulfur. In some embodiments, R² is optionally substituted 5-membered heteroaryl having 1 heteroatom selected from nitrogen, oxygen, and sulfur. In some embodiments, R³ is optionally substituted thiophenyl. In some embodiments, R³ is thiophenyl. In some embodiments, R³ is thiophenyl optionally substituted with 1 to 3 substituents independently selected from methyl, ethyl, fluoro, chloro, bromo, cyclopropyl, —OCH₃, —SCH₃, —NH₂, —NO₂, —CF₃, —CN, —C≡CH, —CH₂—CH═CH₂, —OH, and —NHC(O)CH₃.

In some embodiments, R³ is optionally substituted 6-membered heteroaryl having 1-2 heteroatoms independently selected from nitrogen, oxygen, and sulfur. In some embodiments, R³ is optionally substituted 6-membered heteroaryl having 2 nitrogen atoms. In some embodiments, R³ is optionally substituted pyridinyl. In some embodiments, R³ is pyridinyl. In some embodiments, R³ is pyridinyl optionally substituted with one or more groups selected from —(CH₂)₀₋₄R^{∘} and halogen. In some embodiments, R³ is pyridinyl optionally substituted with one or more —(CH₂)₀₋₄R^{∘}. In some embodiments, R³ is pyridinyl optionally substituted with one or more —R^{∘}. In some embodiments, R^{∘} is C₁₋₆ aliphatic. In some embodiments, R^{∘} is C₁₋₃ aliphatic. In some such embodiments, R^{∘} is methyl.

In some embodiments, R³ is pyridinyl optionally substituted with one or more halogen. In some such embodiments, halogen is selected from fluoro and chloro. In some embodiments, halogen is fluoro. In some embodiments, halogen is chloro. In some embodiments, halogen is selected from bromo and iodo. In some embodiments, halogen is bromo. In some embodiments, halogen is iodo.

In some embodiments, R³ is pyridinyl optionally substituted with 1 to 3 substituents independently selected from methyl, ethyl, fluoro, chloro, bromo, cyclopropyl, —OCH₃, —SCH₃, —NH₂, —NO₂, —CF₃, —CN, —C≡CH, —CH₂—CH═CH₂, —OH, and —NHC(O)CH₃. In some embodiments, R³ is selected from 4-chloropyridin-3-yl, 2-chloro-4-methylpyridin-3-yl, and 2,4-dimethylpyridin-3-yl.

In some embodiments, R³ is pyridinyl having 1 to 3 substituents independently selected from those set forth above.

In some embodiments, R³ is

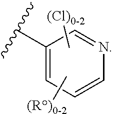

In some embodiments, R³ is selected from

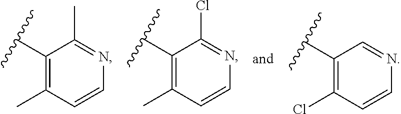

In some embodiments, R³ is optionally substituted heteroaryl other than tetrazolyl wherein two substituents on the heteroaryl are optionally taken together to form a heterocyclic or heteroaryl ring fused to the heteroaryl. In some embodiments, R³ is optionally substituted 1H-indazolyl. In some embodiments, R³ is 1H-indazolyl. In some embodiments, R³ is 1H-indazolyl optionally substituted with one or more —(CH₂)₀₋₄R^{∘}. In some embodiments, R³ is 1H-indazolyl optionally substituted with one or more —R^{∘}. In some embodiments, R^{∘} is C₁₋₆ aliphatic. In some embodiments, R^{∘} is C₁₋₃ aliphatic. In some such embodiments, R^{∘} is methyl.

In some embodiments, R³ is 1H-indazolyl optionally substituted with 1 to 3 substituents independently selected from methyl, ethyl, fluoro, chloro, bromo, cyclopropyl, —OCH₃, —SCH₃, —NH₂, —NO₂, —CF₃, —CN, —C≡CH, —CH₂—CH═CH₂, —OH, and —NHC(O)CH₃. In some embodiments, R³ is 5-methyl-1H-indazol-4-yl.

In some embodiments, R³ is indazolyl having 1 to 3 substituents independently selected from those set forth above.

In some embodiments, R³ is

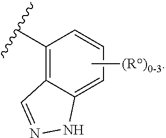

In some embodiments, R³ is

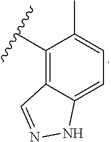 [SMILES: *c1c(C)ccc2[nH]ncc12]

In some embodiments, R³ is selected from phenyl, thiophenyl, pyridinyl, and 1H-indazolyl. In some such embodiments, R³ is substituted with 1 to 3 substituents independently selected from methyl, ethyl, fluoro, chloro, bromo, cyclopropyl, —OCH₃, —SCH₃, —NH₂, —NO₂, —CF₃, —CN, —C≡CH, —CH₂—CH═CH₂, —OH, and —NHC(O)CH₃.

In some embodiments, R³ is selected from 4-chloropyridin-3-yl, 2,6-dichloro-4-fluorophenyl, 2,3-difluoro-6-chlorophenyl, 2,3-difluoro-6-chlorophenyl, 2,4-difluoro-6-chlorophenyl, 2,5-difluoro-6-chlorophenyl, 2-bromo-6-chlorophenyl, 2-bromo-6-fluorophenyl, 2,6-dichloro-3-hydroxyphenyl, 2,6-dichlorophenyl, 2-fluoro-6-chlorophenyl, 2-bromophenyl, 2-chloro-5-hydroxyphenyl, 2-chlorophenyl, 2-chloro-4-methylpyridin-3-yl, 3-hydroxyphenyl, phenyl, 2-bromo-6-cyanophenyl, 2-chloro-6-trifluoromethylphenyl, 2-chloro-5-cyanophenyl, 2-chloro-6-cyanophenyl, 2-trifluoromethylphenyl, 2-cyanophenyl, 2-bromo-6-methylphenyl, 2-methyl-3-hydroxy-6-chlorophenyl, 2-chloro-6-methylphenyl, 2-methyl-5-nitrophenyl, 2,4-dimethylpyridin-3-yl, 2-methyl-5-aminophenyl, 2-chloro-6-ethynylphenyl, 2-ethynylphenyl, 2-methyl-6-trifluoromethylphenyl, 2-chloro-6-ethylphenyl, 2,6-dimethyl-3-hydroxyphenyl, 2-methyl-5-methylcarbamylphenyl, 2,6-dichloro-3-dimethylaminocarbonyloxyphenyl, and 5-methyl-1H-indazol-4-yl.

As defined generally above and discussed throughout, R⁴ is hydrogen or C₁-C₄ alkyl. In some embodiments, R⁴ is hydrogen.

In some embodiments, R⁴ is C₁-C₄ alkyl.

In some embodiments, the present disclosure provides a compound of any of formulae I-a, I-b, I-c, I-d, I-e, or I-f:

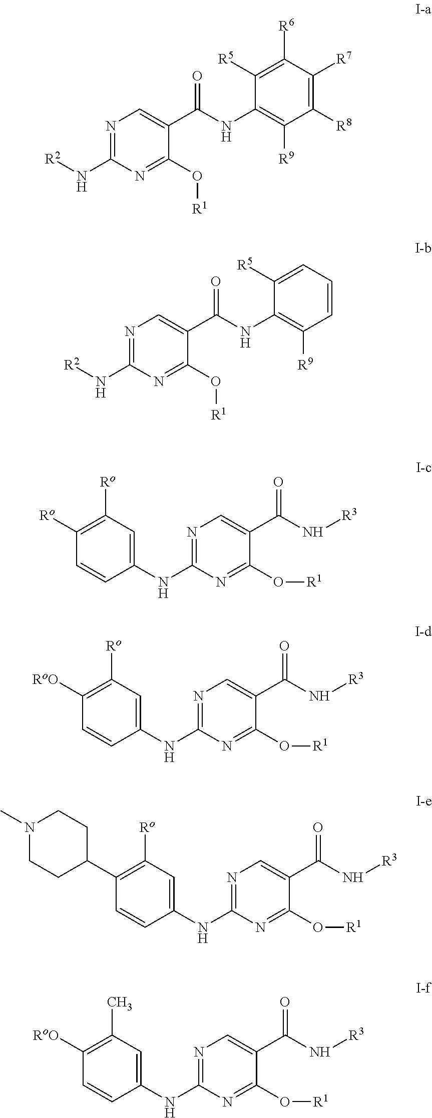

or a pharmaceutically acceptable salt thereof.

### 4. Uses, Formulation and Administration

### Pharmaceutically Acceptable Compositions

According to another embodiment, the disclosure provides a composition comprising a compound of this disclosure or a pharmaceutically acceptable derivative thereof and a pharmaceutically acceptable carrier, adjuvant, or vehicle. The amount of compound in compositions of this disclosure is such that is effective to measurably inhibit Wee1A kinase, or a mutant thereof, in a biological sample or in a patient. In certain embodiments, a composition of this disclosure is formulated for administration to a patient in need of such composition. In some embodiments, a composition of this disclosure is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration.

The amount of compounds of the present disclosure that may be combined with the carrier materials to produce a composition in a single dosage form will vary depending upon the host treated, the particular mode of administration. Preferably, provided compositions should be formulated so that a dosage of between 0.001-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Compounds and Pharmaceutically Acceptable Compositions

Compounds and compositions described herein are generally useful for the inhibition of protein kinase activity of one or more enzymes.

Examples of kinases that are inhibited by the compounds and compositions described herein and against which the methods described herein are useful include Wee1A kinase.

The activity of a compound utilized in this disclosure as an inhibitor of Wee1A kinase, or a mutant thereof, may be assayed in vitro, in vivo or in a cell line. In vitro assays include assays that determine inhibition of either the phosphorylation activity and/or the subsequent functional consequences, or ATPase activity of activated Wee A kinase, or a mutant thereof. Alternate in vitro assays quantitate the ability of the inhibitor to bind to Wee1A kinase.

The inhibition of the DNA damage response (DDR) pathway in the treatment of cancer has recently gained interest, and different DDR inhibitors have been developed. Among them, the most promising ones target the Wee1 kinase family, which has a crucial role in cell cycle regulation and DNA damage identification and repair in both nonmalignant and cancer cells.

### Wee1 Kinase Family

The Wee1 kinase family consists of three serine/threonine kinases sharing conserved molecular structures and encoded by the following genes: WEE1 (Wee1A kinase or Wee1 G2 checkpoint kinase), PKMYT1 (Myt1 kinase or membrane-associated tyrosine- and threonine-specific cdc2-inhibitory kinase), and WEE2 (Wee1B kinase or WEE oocyte meiosis inhibiting kinase). In eukaryotic somatic cells, Wee1A kinase and Myt1 kinase play a key role in cell cycle regulation, in particular, in the entry into mitosis (Schmidt M, Robe A, Platzer C, et alt Regilation of G2/M transition by inhibition of Wee and PMyt1 Kinases. Molecules. 2017:22:2045). Their role as regulators is crucial during normal cell cycle progression and in response to DNA damage as part of the DNA damage response (DDR) pathways. Similarly, Wee1B kinase regulates cell cycle progression and, in particular, meiosis (Solc P, Schultz R M, Motlik J. Prophase I arrest and progression to retaphase I in mouse oocytes: Comparison of resumption of mveiosis and recovery from G2-arrest in somatic cells, Mol Hum Reprod. 2010:16:654-64).

### Wee1B Kinase

Wee1B kinase expression is germ-cell specific and inhibits meiosis by phosphorylating Tyr15 of the CDK1-cyclin B complex (JY Zhu et al., J Med Chem. 2017; 60 (18), 7863-7875). Previous and current drug discovery efforts have not been focused on Wee1B kinase due to its characterized role in cell-cycle regulation. Wee1B kinase plays a dual regulatory role in oocyte meiosis by preventing premature restart prior to ovulation and permitting metaphase II exit at fertilization (Nakanishi M, Ando I, Watanabe N, et al. Identification and characterization of human Wee B, a new member of the Wee1 family of Cdk-inhibitory kinases. Genes Cells. 2000:5(10):839-47). Despite the identification of WEE2 somatic mutations (1.9% of cases) and copy number (CN) alterations (22.5% of patients with CN loss and 22.5% with CN gain) across several cancer types (https://portal.gdc.cancer.gov), they have not yet been functionally linked to tumor development.

### Myt1 Kinase

Myt1 kinase is a multi-functional protein kinase localized to the ER-Golgi complex that is known to play a regulatory role in the cell cycle by inhibiting Cdk1/cyclin B1 mediated mitosis (JY Zhu et al., J Med Chem. 2017; 60 (18), 7863-7875). As mentioned above and throughout, Myt1 kinase inhibits the Cdk1/cyclin B1 interaction through the phosphorylation of Tyr15 and Thr14 of Cdk1 and sequestration of Cdk1 from the nucleus. Additionally, Myt1 kinase has been tied to orchestrating the ER-Golgi complex reassembly during mitotic exit.

### Wee1A Kinase

Wee1A kinase regulates entry into mitosis at the G2/M transition of the S phase by phosphorylating Tyr15 of Cdk1 to inactive the Cdk1/cyclin B complex. Cells with perturbed G1 checkpoint activity (e.g., cancer cells) rely on Wee1A kinase to inhibit Cdk1 to permit a G2/M arrest for DNA repair. If Wee1A kinase activity is altered, a perturbed cell may enter mitosis prematurely without having the opportunity to fully replicate the entire DNA content or repair potential DNA that might have occurred during S phase. This characterization of Wee1A kinase's role in the cell cycle has made it an attractive target for anticancer therapeutics, especially in combination with DNA-damaging agents (JY Zhu et al., J Med Chem. 2017; 60 (18), 7863-7875).

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of Wee1A kinase and are therefore useful for treating one or more disorders associated with activity of Wee1A kinase. Thus, in certain embodiments, the present disclosure provides a method for treating a Wee1A kinase-mediated disorder comprising the step of administering to a patient in need thereof a compound of the present disclosure, or pharmaceutically acceptable composition thereof.

As used herein, the term “Wee1A kinase-mediated” disorder or condition as used herein means any disease or other deleterious condition in which Wee1A kinase, or a mutant thereof, is known to play a role. Accordingly, another embodiment of the present disclosure relates to treating or lessening the severity of one or more diseases in which Wee1A kinase, or a mutant thereof, is known to play a role. Specifically, the present disclosure relates to a method of treating or lessening the severity of a disease or condition selected from a proliferative disorder, wherein said method comprises administering to a patient in need thereof a compound or composition according to the present disclosure.

In some embodiments, the present disclosure provides a method of inhibiting Wee1A kinase activity in a subject comprising the step of administering to the subject an effective amount of a compound, or a pharmaceutically acceptable composition, of the present disclosure.

In some embodiments, the present disclosure provides a method for treating or lessening the severity of one or more disorders selected from a cancer comprising the step of administering to the subject an effective amount of a compound, or a pharmaceutically acceptable composition thereof, of the present disclosure. In some embodiments, the cancer is associated with a solid tumor.

In some embodiments, the present disclosure provides a method of treating a subject suffering from a cancer or other disordered cell growth characterized by aberrant Wee1A kinase activity comprising the step of administering to the subject an effective amount of a compound, or a pharmaceutically acceptable composition thereof, of the present disclosure. In some embodiments, aberrant Wee1A kinase activity includes elevated activity, or overexpression, or undesirable activity as compared to a non-diseased state. In some such embodiments, aberrant Wee1A kinase activity may include perturbed p53 activity, Cdk1 activity, Cdk2 activity, altered mitosis, and DNA damage. In some embodiments, the subject is suffering from a cancer associated with inactivation of p53. In some such embodiments, the cancer is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma. In some embodiments, the subject is suffering from a cancer selected from a uterine serous carcinoma and a renal cancer.

In some embodiments, the present disclosure provides a method for treating or lessening the severity of one or more disorders selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma. In some embodiments, the disorders are selected from uterine serous carcinoma and a renal cancer.

In some embodiments, the breast cancer is selected from ductal carcinoma in situ (DCIS), invasive ductal carcinoma (IDC), lobular carcinoma in situ (LCIS), invasive lobular cancer (ILC), triple negative breast cancer (TNBC), inflammatory breast cancer (IBC), metastatic breast cancer (MBC), medullary carcinoma, tubular carcinoma, mucinous carcinoma (colloid), and Paget disease of the breast or nipple (commonly known as Paget disease).

In some embodiments, the uterine cancer is selected from endometrial cancer and uterine sarcoma. In some embodiments, the uterine cancer is endometrial cancer. In some embodiments, the uterine cancer is uterine sarcoma.

In some embodiments, the ovarian cancer is selected from epithelial ovarian carcinomas, germ cell tumors, and stromal cell tumors.

In some embodiments, the stomach cancer is selected from adenocarcinoma, lymphoma, gastrointestinal stromal tumors (GISTs), carcinoid tumors, and hereditary (familial) diffuse gastric cancer.

In some embodiments the esophageal cancer is selected from squamous cell carcinoma, small cell carcinoma, and adenocarcinoma. In some embodiments the esophageal cancer is selected from squamous cell carcinoma and adenocarcinoma. In some embodiments, the esophageal cancer is squamous cell carcinoma. In some embodiments, the esophageal cancer is adenocarcinoma.

In some embodiments, the lung cancer is selected from non-small cell lung cancer, lung nodules, small cell lung cancer, and mesothelioma. In some embodiments, the lung cancer is non-small cell lung cancer.

In some embodiments the colorectal cancer is selected from adenocarcinoma, gastrointestinal stromal tumors (GIST), lymphoma, carcinoids, Turcot syndrome, Peutz-Jeghers syndrome (PJS), familial colorectal cancer (FCC), and juvenile polyposis coli.

In some embodiments, the cancer is associated with deregulation of cyclin E1. In some embodiments, the cancer associated with deregulation of cyclin E1 is ovarian cancer.

In some embodiments, the cancer is associated with deregulation of p53. In some embodiments, the cancer associated with deregulation of p 53 is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma. In some such embodiments, the cancer associated with deregulation of p53 is selected from uterine serous carcinoma and a renal cancer.

In some embodiments, the cancer is associated with deregulation of Cdk1. In some embodiments, the cancer associated with deregulation of Cdk1 is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma. In some such embodiments, the cancer associated with deregulation of Cdk1 is selected from uterine serous carcinoma and a renal cancer.

In some embodiments, the cancer is associated with deregulation of Cdk2. In some embodiments, the cancer associated with deregulation of Cdk2 is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma. In some such embodiments, the cancer associated with deregulation of Cdk1 is selected from uterine serous carcinoma and a renal cancer.

Depending upon the particular condition, or disease, to be treated, additional therapeutic agents, which are normally administered to treat that condition, may also be present in the compositions of this disclosure. As used herein, additional therapeutic agents that are normally administered to treat a particular disease, or condition, are known as “appropriate for the disease, or condition, being treated.”

For example, compounds of the present disclosure, or a pharmaceutically acceptable composition thereof, are administered in combination with chemotherapeutic agents to treat proliferative diseases and cancer. Examples of known chemotherapeutic agents include, but are not limited to, Adriamycin, dexamethasone, vincristine, cyclophosphamide, fluorouracil, topotecan, taxol, interferons, platinum derivatives, taxane (e.g., paclitaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, emetine, trimetrexate, metoprine, cyclosporine, daunorubicin, teniposide, amphotericin, alkylating agents (e.g., chlorambucil), 5-fluorouracil, camptothecin, cisplatin, metronidazole, and Gleevec™, among others. In other embodiments, a compound of the present disclosure is administered in combination with a biologic agent, such as Avastin or VECTIBIX.

In some embodiments, compounds of the present disclosure or a pharmaceutically acceptable composition thereof, are administered in combination with an agent selected from fasudil, sirolimus, imatinib, gefitinib, erlotinib, sorafenib, sunitinib, dasatinib, lapatinib, nilotinib, temsirolimus, everolimus, pazopanib, ruxolitinib, vandetanib, vemurafenib, crizotinib, icotinib, axitinib, tofacitinib, bosutinib, cabozantinib, ponatinib, regorafenib, afatinib, dabrafenib, trametinib, ibrutinib, nintedanib, idelalisib, ceritinib, apatinib rivoceranib, ripasudil, alectinib, cobimetinib, lenvatinib, palbociclib, radotinib, osimertinib, olmutinib, neratinib, ribociclib, copanlisib, abemaciclib, acalabrutinib, midostaurin, brigatinib, baricitinib, netarsudil, tivozanib, simotinib, fostamatinib, encorafenib, binimetinib, catequentinib, duvelisib, dacomitinib, lorlatinib, larotrectinib, gilteritinib, pyrotinib, fruquintinib, erdafitinib, alpelisib, umbralisib, leniolisib, pexidartinib, entrectinib, upadacitinib, fedratinib, zanubrutinib, flumatinib, peficitinib, delgocitinib, avapritinib, selumetinib, tucatinib, pemigatinib, capmatinib tabrecta, selpercatinib, ripretinib, tirabrutinib, almonertinib, pralsetinib, filgotinib, tirbanibulin, orelabrutinib, tepotinib, and trilaciclib. See List of clinically approved kinase inhibitors|MRC Protein Phosphorylation Ubiquitylation Unit available at www.ppu.mrc.ac.uk/list-clinically-approved-kinase-inhibitors, incorporated herein by reference in its entirety.

In certain embodiments, compounds of the present disclosure, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG Live, bevacizumab, fluorouracil, bexarotene, bleomycin, bortezomib, busulfan, calusterone, capecitabine, camptothecin, carboplatin, carmustine, celecoxib, cetuximab, chlorambucil, cladribine, clofarabine, cyclophosphamide, cytarabine, dactinomycin, darbepoetin alfa, daunorubicin, decitabine, denileukin, dexrazoxane, docetaxel, doxorubicin (neutral), doxorubicin hydrochloride, dromostanolone propionate, epirubicin, epoetin alfa, erlotinib, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

In certain embodiments, compounds of the present disclosure, or a pharmaceutically acceptable composition thereof, are co-administered with a pharmaceutically acceptable Myt1 kinase inhibitor. In some such embodiments, the Myt1 kinase inhibitor is RP-6306.

In certain embodiments, compounds of the present disclosure, or a pharmaceutically acceptable composition thereof, are co-administered with a pharmaceutically acceptable DNA damaging agent.

In certain embodiments, compounds of the present disclosure, or a pharmaceutically acceptable composition thereof, are co-administered with radiation.

In certain embodiments, compounds of the present disclosure, or a pharmaceutically acceptable composition thereof, are administered in combination with a monoclonal antibody or an siRNA therapeutic.

In certain embodiments, compounds of the present disclosure, or a pharmaceutically acceptable composition thereof, are administered in combination with a targeted therapy selected from (i) an inhibitor of a kinase selected from MET, MEK, mTOR, FLT3, BRAF, KIT, PDGFR, FDFR, PI3K, EGFR, AKT, and KRAS, (ii) an inhibitor of a fusion kinase like BCR-ABL, ALK, RET and ROS, JAK, CDK4/6, and KRAS, (iii) epigenetic modulators such as an HDAC inhibitor, (iv) immuno-oncology agents such as those targeting PD1, PDL1, and CTLA4, (v) antibody drug conjugates such as those targeting Her2, CD38, BCMA, CD19, nectin4, trop2, CD79, and CD22, (vi) bispecific T cell engagers (BiTEs), (vii) transcription factor modulators such as those targeting IKZF (i.e., IMiDs, and EZH₂), (viii) steroid receptor modulators such as those targeting AR and ER, and (ix) proteasome inhibitors such as those targeting PARP, IDH1, IDH2, HDACs, and BCL2.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another, for example, within one, two, three, four, five, six, seven, eight, nine, ten, eleven, or twelve hours from one another.

In some such embodiments, compounds of the present disclosure, or a pharmaceutically acceptable composition thereof, are administered as part of a multiple dosage regimen with a pharmaceutically acceptable Myt1 kinase inhibitor. In certain embodiments, compounds of the present disclosure, or a pharmaceutically acceptable composition thereof, are administered as part of a multiple dosage regimen with a Myt1 kinase inhibitor selected from RP-6306.

In some embodiments, compounds of the present disclosure, or a pharmaceutically acceptable salt thereof, are administered to a subject wherein a Myt1 kinase inhibitor is used as the first or second line therapy. In some embodiments, compounds of the present disclosure, or a pharmaceutically acceptable salt thereof, are administered to a subject wherein a Myt1 kinase inhibitor is used as a first line therapy. In some embodiments, compounds of the present disclosure, or a pharmaceutically acceptable salt thereof, are administered to a subject wherein a Myt1 kinase inhibitor selected from RP-6306, is used as a first line therapy.

In some embodiments, compounds of the present disclosure, or a pharmaceutically acceptable salt thereof, are administered to a subject wherein a Myt1 kinase inhibitor is used as a second line therapy. In some embodiments, compounds of the present disclosure, or a pharmaceutically acceptable salt thereof, are administered to a subject wherein a Myt1 kinase inhibitor selected from RP-6306, is used as a second line therapy.

As used herein, the term “combination,” “combined,” “co-administered” and related terms refer to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

The amount of both, an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this disclosure should be formulated so that a dosage of between 0.001-100 mg/kg body weight/day of an inventive can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions a dosage of between 0.001-1,000 g/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In some embodiments, the present disclosure provides a method for inhibiting Wee1A kinase in vitro. In some such embodiments, the amount of Wee1A kinase inhibition is assessed based on a competitive ATP-binding assay.

In some embodiments, the present disclosure provides a method for inhibiting Wee1A kinase in a biological sample.

In some embodiments, the present disclosure provides a method for assessing Cdk1 phosphorylation in a cell, comprising contacting said cell with a compound described herein. In one embodiment, the contacting step comprises incubating a cell with a compound presented herein. In some such embodiments, the cell is incubated for at least 4 hours. In some embodiments, the cell may be a ACHN renal carcinoma cell. In some embodiments, the cell may be a DAOY cell.

### EXAMPLES

### General Methods

¹H NMR and ¹³C NMR spectra were recorded on a 500 MHz (¹H NMR at 500 MHz and ¹³C NMR at 126 MHz) Bruker Avance Neo spectrometer equipped with a 5 mm iProbe BBF/H/D probe or on a 400 MHz (¹H NMR at 400 MHz and ¹³C NMR at 101 MHz) Varian Inova spectrometer equipped with a 5 mm 1H/13C auto-switchable gradient-probe at 25° C. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references.

Flash chromatography was performed on a Biotage Isolera One system equipped with a diode array detector using prepacked silica columns (Biotage Sfur 60 μm). UV traces were recorded between 200 and 400 nm. Unless stated otherwise, starting materials were commercially available.

Prep HPLC was performed on instrument Shimadzu, Model:-LC-, MS-2020 or Agilent, Model:-1290 Infinitty II.

SFC: SFC analytical was recorded on Shimadzu, Model:-LC-30AD and SFC purification was performed on PIC, Prep-175&400.

Unless stated otherwise, starting materials were commercially available. All solvents and commercial reagents were of laboratory grade and were used as received. The organic phases from extractions were dried over anhydrous sodium sulfate if not stated otherwise. Organic phases or solutions were concentrated by rotary evaporation. Yields were not optimized.

The following methods was used for LC-MS analysis:

Method A: using a Zorbax Eclipse Plus C18 (50×2.6 mm, 1.8 μm) column at 40° C. with mobile phase A (water with 0.1% formic acid) and B (acetonitrile) under a gradient from 5 to 95% B over 3.5 minutes at a flow rate of 0.8 ml/minute. Mass spectrometer Shimadzu (Model No: LC-30MS₂020) operating in ES (+ or −).

Method B: using Phenomenex Kinetex EVO C18 (3×50 mm, 2.6 μm) column at 40° C. with mobile phase A (water with 10 mM ammonium bicarbonate) and B (acetonitrile) under a gradient from 5 to 98% B over 3.55 minutes at a flow rate of 0.8 ml/minute. Mass spectrometer operating in ES (+ or −).

Method C: was performed on an Agilent 1100 system coupled with an Agilent MSD mass spectrometer operating in ES (+) ionization mode, using a Gemini NX—C18, 3.0×50 mm, 110 Å, column and eluted with solution A (water with 0.1% TFA) and B (acetonitrile) at 40° C. UV-traces were recorded between 220 and 380 nm.

Method D: LC-MS analyses were performed on an Agilent 1260 Infinity II system coupled with an Agilent MSD XT mass spectrometer operating in ES (+ or −) ionization mode, using a Phenomenex Gemini NX—C18, 3.0×50 mm, 110 Å, column and eluted with solution A (water with 0.2% NH₄OH) and B (acetonitrile). UV-traces were recorded at 220 and/or 254 nm.

In the cases where stereoisomers were separated using chromatography (i.e. Supercritical Fluid Chromatography) the resulting stereochemistry was assigned arbitrarily to S or R and the actual configuration on the stereochemistry is unknown.

The following methods were used for HPLC analysis:

Method A: using a X-select CSH C18 (150×4.6 mm, 5.0 μm) column with mobile phase A (water with 0.1% formic acid) and B (acetonitrile) under a gradient from 5 to 100% B over 8 minutes at a flow rate of 2 ml/minute. Detection was performed with a DAD detector @210-400 nm.

Method B: using a X-select CSH C18 (150×4.6 mm, 5.0 μm) column with mobile phase A (water with 0.1% trifluoroacetic acid) and B (acetonitrile) under a gradient from 5 to 100% B over 8 minutes at a flow rate of 2 ml/minute. Detection was performed with a DAD detector @210-400 nm.

Method C: Agilent 1100 system using a Kromasil Eternity-5-C18, 4.6×150 mm column and eluted with solution A (water with 0.1% TFA) and B (acetonitrile with 0.1% TFA) under a gradient from 10-90% B during 13 minutes at a flow of 1 ml/min. UV-traces were recorded at 220 and 254 nm. 220 nm was used for purity analysis.

### Abbreviations

BOC-anhydride di-tert-butyl dicarbonate
n-BuLi n-butyl lithium
DCM dichloromethane
DIPEA N,N-diisopropylethylamine
DMAP 4-dimethylaminopyridine
DMF N,N-dimethylformamide
DMSO dimethylsulfoxide
Et ethyl
EtOAc ethyl acetate
EtOH ethanol
GC-MS gas chromatography-mass spectrometry
LDA lithium diisopropylamide
mCPBA mneta-chloroperbenzoic acid
MeOH methanol
MTBE methyl tert-butyl ether
NCS N-chlorosuccinimide
NBS N-bromosuccinimide
NMP N-methylpyrrolidine
LC-MS liquid chromatography-mass spectroscopy
PdCl₂×dppf 1,1′-bis(diphenylphosphino)ferrocene palladium(II)dichloride
pTSA p-toluenesulfonic acid
RT room temperature, normally 20 to 22° C.
TBAF tetrabutylammonium fluoride
TBS tert-Butyldimethylsilyl
TBSCl tert-Butyldimethylsilyl chloride
TEA triethylamine
TFA trifluoroacetic acid
TFAA trifluoroacetic acid anhydride
TFE 2,2,2-Trifluoroethanol
THE tetrahydrofuran
TBME tert-butyl methyl ether
TLC thin layer chromatography
tᵣ or Tᵣₑₜ retention time
Triflic anhydride trifluoromethanesulfonic anhydride (Tf₂O)
UPLC Ultra high performance liquid chromatography

### Example 1. Synthesis of N-(2,6-dichlorophenyl)-4-methoxy-2-((3-methyl-4-(1-methylpiperidin-4-yl)phenyl)amino)pyrimidine-5-carboxamide (Compound 100)

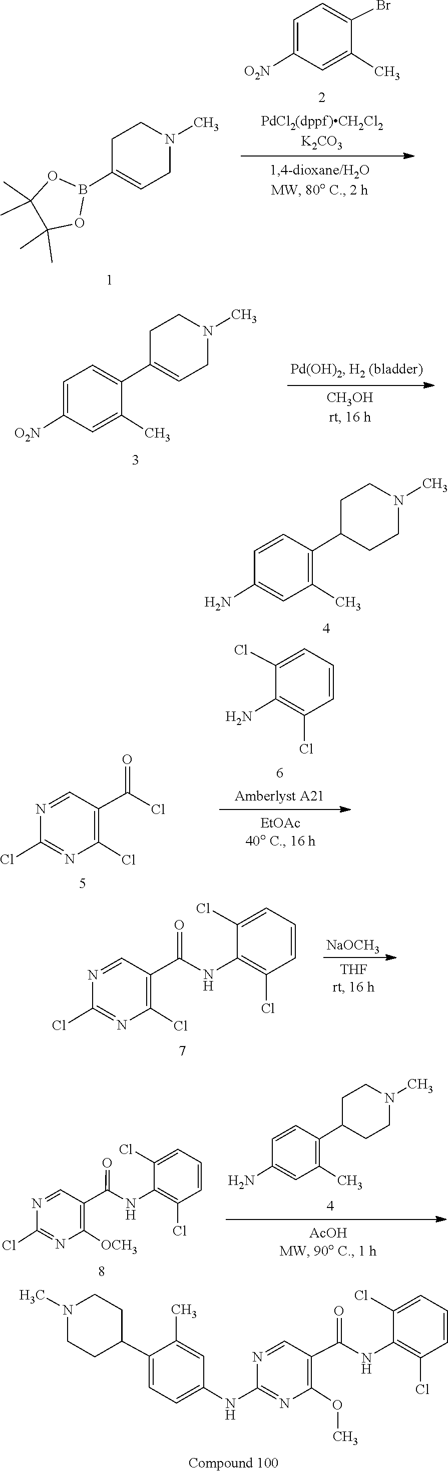

### 1-methyl-4-(2-methyl-4-nitrophenyl)-1,2,3,6-tetrahydropyridine

To a stirred solution of 1-bromo-2-methyl-4-nitrobenzene (500 mg, 2.314 mmol) and 1-methyl-4-(4,4,5,5-tetramethyl-1,3,2-dioxaborolan-2-yl)-1,2,3,6-tetrahydropyridine (749 mg, 3.36 mmol) in 1,4-dioxane (30 mL), water (8 mL) was added potassium carbonate (958 mg, 6.94 mmol) at room temperature in a microwave vial. After degassing with argon for 5 min., Pd(dppf)Cl₂·CH₂Cl₂ (28.3 mg, 0.035 mmol) was added to the reaction mixture under argon atmosphere. The resulting reaction mixture was subjected to microwave irradiation at 80° C. for 2 h. After completion of the reaction (TLC and UPLC), the mixture was filtered through celite, the filtrate was diluted with water, and extracted with ethyl acetate (30 mL×2). The combined organic layer was dried over Na₂SO₄, filtered and concentrated under reduced pressure. The resulting crude material was purified by flash chromatography (SiO₂/100-200 mesh; ~5-7% CH₃OH/CH₂Cl₂) to afford 1-methyl-4-(2-methyl-4-nitrophenyl)-1,2,3,6-tetrahydropyridine (420 mg, 78% yield) as a brown gum. ¹H-NMR (DMSO-d₆, 400 MHz): δ 8.10 (d, J=2.4 Hz, 1H), 8.03-8.00 (m, 2H), 7.36 (d, J=8.4 Hz, 1H), 5.67-5.64 (m, 1H), 3.93 (s, 1H), 3.04-3.02 (m, 2H), 2.62-2.53 (m, 2H), 2.37 (s, 3H), 2.31 (s, 3H). Chemical Formula: C₁₃H₁₆N₂O₂, Exact Mass: 232.12, Molecular Weight: 232.28.

### 3-methyl-4-(1-methylpiperidin-4-yl)aniline

To a degassed solution of 1-methyl-4-(2-methyl-4-nitrophenyl)-1,2,3,6-tetrahydropyridine (500 mg, 1.937 mmol) in methanol (25 mL) was added 10% Pd(OH)₂ (180 mg) under nitrogen atmosphere. The reaction mixture was stirred under hydrogen atmosphere using hydrogen bladder pressure at room temperature for 16 h. After completion of the reaction (TLC and UPLC), the reaction mixture was filtered under nitrogen atmosphere through a pad of celite to remove catalyst and the celite pad was washed with methanol (80 mL). The filtrate was concentrated under reduced pressure to get 3-methyl-4-(1-methylpiperidin-4-yl)aniline (380 mg, 96% yield) as a brown gum, which was taken into the next step without further purification. ¹H-NMR (DMSO-d₆, 400 MHz): δ 6.82 (d, J=8.0 Hz, 1H), 8.40-8.35 (m, 2H), 4.72 (br s, 2H), 2.87 (d, J=11.6 Hz, 2H), 2.46-2.42 (m, 1H), 2.21 (s, 3H), 2.14 (s, 3H), 2.03-1.97 (m, 2H), 1.59-1.54 (m, 4H). Chemical Formula: C₃H₂₀N₂, Exact Mass: 204.16, Molecular Weight: 204.32. LCMS (ESI) m/z=205.2 (M+H), t_{R}. 1.084 min, 91.2% (Method B).

### 2,4-dichloro-N-(2,6-dichlorophenyl)pyrimidine-5-carboxamide

To a stirred solution of 2,4-dichloropyrimidine-5-carbonyl chloride (0.6 mL, 4.73 mmol) in ethyl acetate (80 mL), was added 2,6-dichloroaniline (0.613 g, 3.78 mmol) followed by the addition of Amberlyst A 21 Free base (200 mg, 4.73 mmol) at room temperature under nitrogen atmosphere. Then, the reaction mixture was allowed to stir at 40° C. for 16 h. After completion of the reaction (TLC and UPLC), the reaction mixture was diluted with water and extracted with ethyl acetate (50 mL×2). The combined organic layer was dried over Na₂SO₄, filtered and concentrated under reduced pressure. The resulting crude material was washed with MTBE to get 2,4-dichloro-N-(2,6-dichlorophenyl)pyrimidine-5-carboxamide (600 mg, 38% yield) as an off-white solid. ¹H-NMR (DMSO-d₆, 400 MHz): δ 10.85 (s, 1H), 9.02 (s, 1H), 7.63 (d, J=8.0 Hz, 2H), 7.45 (t, J=8.0 Hz, 1H). Chemical Formula: C₁₁H₅Cl₄N₃O, Exact Mass: 334.92, Molecular Weight: 336.98. LCMS (ESI) m/z=334.0/337.0/338.0 (M−H) chlorine isotopes, t_{R}. 1.994 min, 97.2% (Method A).

### 2-chloro-N-(2,6-dichlorophenyl)-4-methoxypyrimidine-5-carboxamide

To a stirred solution of 2,4-dichloro-N-(2,6-dichlorophenyl)pyrimidine-5-carboxamide (600 mg, 1.781 mmol) in tetrahydrofuran (10 mL), was added sodium methoxide (192 mg, 3.56 mmol) at room temperature. The resulting reaction mixture was stirred at room temperature for 16 h. After completion of the reaction (TLC and UPLC), the reaction mixture was diluted with water and extracted with dichloromethane (50 mL×2). The combined organic layer was dried over Na₂SO₄, filtered and concentrated under reduced pressure to get 2-chloro-N-(2,6-dichlorophenyl)-4-methoxypyrimidine-5-carboxamide (500 mg, 84% yield) as an off-white solid. ¹H-NMR (DMSO-d₆, 400 MHz): δ 10.2 (s, 1H), 8.82 (s, 1H), 7.60 (d, J=8.0 Hz, 2H), 7.42 (t, J=8.0 Hz, 1H), 4.10 (s, 3H). Chemical Formula: C₁₂H₈Cl₃N₃O₂, Exact Mass: 330.97, Molecular Weight: 332.57. LCMS (ESI) ni/z=332.2/334.0/335.0 (M+H) chlorine isotopes, t_{R}. 2.004 min, 74.6% (Method A).

### N-(2,6-dichlorophenyl)-4-methoxy-2-((3-methyl-4-(1-methyl piperidin-4-yl)phenyl)amino)pyrimidine-5-carboxamide (Compound 100)

To a stirred solution of 2-chloro-N-(2,6-dichlorophenyl)-4-methoxypyrimidine-5-carboxamide (200 mg, 0.601 mmol) in acetic acid (5 mL) was added 3-methyl-4-(1-methylpiperidin-4-yl)aniline (123 mg, 0.601 mmol) at 25° C. under nitrogen atmosphere. The resultant reaction mixture was subjected to microwave irradiation at 90° C. for 1 h. After completion of the reaction (TLC and UPLC), the reaction mixture was diluted with water and extracted with ethyl acetate (50 mL×2). The combined organic layer was dried over Na₂SO₄, filtered and concentrated under reduced pressure. The resulting crude material was purified by flash chromatography (SiO₂/100-200 mesh; 0-10% CH₃OH/CH₂Cl₂) to afford N-(2,6-dichlorophenyl)-4-methoxy-2-((3-methyl-4-(1-methylpiperidin-4-yl)phenyl)amino)pyrimidine-5-carboxamide (Compound 100, 40 mg, 13% yield) as a pale yellow solid. ¹H NMR (400 MHz, DMSO-d₆): δ 10.07 (br s, 1H), 9.56 (s, 1H), 8.75 (s, 1H), 7.62-7.56 (m, 4H), 7.41-7.37 (t, J=8.0 Hz, 1H), 7.13-7.12 (d, J=7.6 Hz, 1H), 4.11 (s, 3H), 3.53-3.43 (m, 2H), 3.04-2.93 (m, 3H), 2.76 (br. s, 2H), 2.34 (s, 3H), 1.99-1.88 (m, 5H). Chemical Formula: C₂₅H₂₇Cl₂N₅O₂, Exact Mass: 499.15, Molecular Weight: 500.42. LCMS (ESI) m/z=500.2 (M⁺), t_{R}. 1.879 min, 98.3% (Method-B), HPLC: 97.2%, t_{R}: 4.504 min, (Method-B).

### Example 2. Synthesis of 4-methoxy-2-((3-methyl-4-(1-methylpiperidin-4-yl)phenyl)amino)-N-phenylpyrimidine-5-carboxamide (Compound 101)

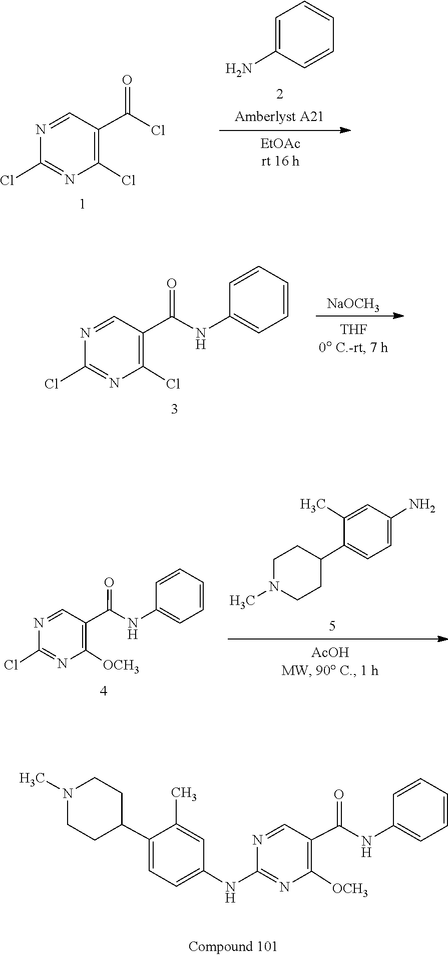

### 2,4-dichloro-N-phenylpyrimidine-5-carboxamide

To a stirred solution of aniline (0.22 mL, 2.365 mmol) in ethyl acetate (15 mL), was added 2,4-dichloropyrimidine-5-carbonyl chloride (0.304 mL, 2.365 mmol) dropwise at room temperature. After 5 min to this white cloudy reaction mixture, was added Amberlyst A 21 resin (50 mg). The resulting reaction mixture was stirred at room temperature for 16 h. After completion of the reaction (TLC and UPLC), the reaction mixture was diluted with water and extracted with ethyl acetate (50 mL×2). The combined organic layer was dried over Na₂SO₄, filtered and concentrated under reduced pressure to afford 2,4-dichloro-N-phenylpyrimidine-5-carboxamide (834 mg, crude) as a yellow solid which was taken to the next step without further purification. ¹H-NMR (DMSO-d₆, 400 MHz): δ 10.77 (s, 1H), 9.10 (s, 1H), 7.69-7.67 (m, 2H), 7.42-7.38 (m, 2H), 7.20-7.15 (m, 1H). Chemical Formula: C₁₁H₇Cl₂N₃O, Exact Mass: 267.00, Molecular Weight: 268.10. LCMS (ESI) m/z=268.0 (M+H)⁺, t_{R}. 1.843 min, 95.5% (Method-A).

### 2-chloro-4-methoxy-N-phenylpyrimidine-5-carboxamide

To a solution of 2,4-dichloro-N-phenylpyrimidine-5-carboxamide (400 mg, 1.492 mmol) in tetrahydrofuran (8 mL), was added sodium methanolate (85 mg, 1.567 mmol) at 0° C. The resulting reaction mixture was stirred for 7 h at room temperature. After completion of the reaction (TLC and UPLC), the mixture was diluted with water and extracted with ethyl acetate (40 mL×2). The combined organic layer was dried over Na₂SO₄, filtered and concentrated under reduced pressure to afford 2-chloro-4-methoxy-N-phenylpyrimidine-5-carboxamide (380 mg, 97% yield) as a yellow solid, which was taken into the next step without purification. Chemical Formula: C₁₂H₁₀ClN₃O₂, Exact Mass: 263.05, Molecular Weight: 263.68. LCMS (ESI) m/z=264.2 (M+H), t_{R}. 1.959 min, 83% (Method-A).

### 4-methoxy-2-((3-methyl-4-(1-methylpiperidin-4-yl)phenyl)amino)-N-phenylpyrimidine-5-carboxamide (Compound 101)

To a stirred solution of 3-methyl-4-(1-methylpiperidin-4-yl)aniline (100 mg, 0.489 mmol) in acetic acid (5 mL) was added 2-chloro-4-methoxy-N-phenylpyrimidine-5-carboxamide (129 mg, 0.489 mmol) at room temperature. The resulting reaction mixture was subjected to microwave irradiation at 90° C. for 1 h. After completion of the reaction (TLC and UPLC), volatiles were removed under reduced pressure and the resulting crude material was purified by flash chromatography (SiO₂/100-200 mesh; 0-10% CH₃OH/CH₂Cl₂) to afford 4-methoxy-2-((3-methyl-4-(1-methylpiperidin-4-yl)phenyl)amino)-N-phenylpyrimidine-5-carboxamide (Compound 101, 19 mg, 9% yield) as a white solid. ¹H-NMR (400 MHz, CD₃OD): δ 8.84 (s, 1H), 7.68-7.65 (m, 2H), 7.63-7.60 (m, 1H), 7.53 (d, J=2 Hz, 1H), 7.40-7.36 (m, 2H), 7.22 (d, J=8.4 Hz, 1H), 7.18-7.14 (m, 1H), 4.23 (s, 3H), 3.60-3.56 (m, 2H), 3.16-3.10 (m, 3H), 2.90 (s, 3H), 2.41 (s, 3H), 2.06-1.95 (m, 4H). Note: Exchangeable protons were not observed. Chemical Formula: C₂₅H₂₉N₅O₂, Exact Mass: 431.23, Molecular Weight: 431.54. LCMS (ESI) m/z=432.4 (M+H), t_{R}. 1.490 min, 97.9% (Method-A); HPLC: 97.2%, t_{R}=3.897 min. (Method-A).

### Example 3. Synthesis of 4-cyclopropoxy-N-(2,6-dichlorophenyl)-2-((4-(4-methylpiperazin-1-yl)phenyl)amino)pyrimidine-5-carboxamide (Compound 102)

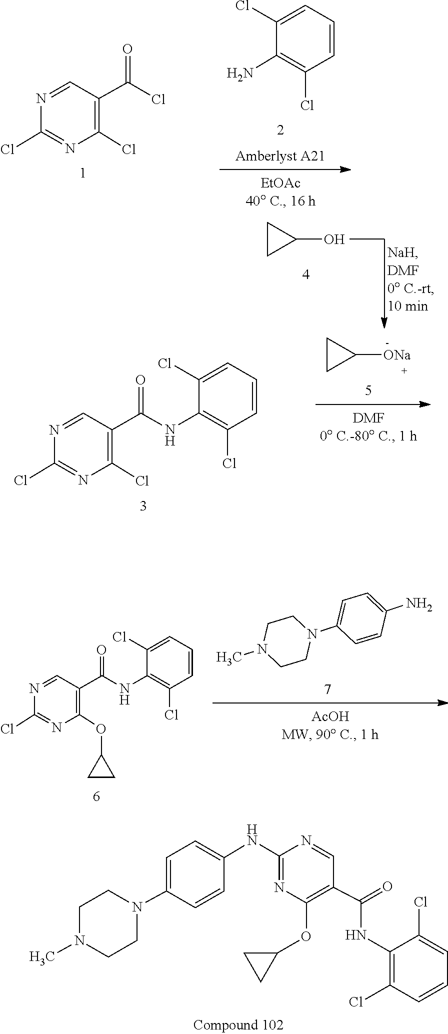

### 2,4-dichloro-N-(2,6-dichlorophenyl)pyrimidine-5-carboxamide

To a stirred solution of 2,4-dichloropyrimidine-5-carbonyl chloride (1 g, 4.73 mmol) in EtOAc (30 mL) was added 2,6-dichloroaniline (0.613 g, 3.78 mmol) followed by Amberlyst A21 (1 g, 4.73 mmol) at room temperature under nitrogen atmosphere. The reaction mixture was stirred at 40° C. for 16 h. After completion of the reaction (TLC and UPLC), the reaction mixture was diluted with water and extracted with EtOAc (60 mL×2). The combined organic layer was dried over Na₂SO₄, filtered and concentrated under reduced pressure. The resulting crude material was washed with methyl tert-butyl ether to get 2,4-dichloro-N-(2,6-dichlorophenyl)pyrimidine-5-carboxamide (550 mg, 34% yield) as an off-white solid. Chemical Formula: C₁₁H₅Cl₄N₃O, Exact Mass: 334.92, Molecular Weight: 336.98. LCMS (ESI) m/z=333.9/337.8 (M−H), chlorine isotopes, t_{R}=1.893 min, 99.5% (Method-A).

### 2-chloro-4-cyclopropoxy-N-(2,6-dichlorophenyl)pyrimidine-5-carboxamide

To a stirred solution of cyclopropanol (43.1 mg, 0.742 mmol) in DMF (5 mL), was added sodium hydride (29.7 mg, 0.742 mmol) at 0° C. The reaction mixture was stirred for 10 min. To this solution, 2,4-dichloro-N-(2,6-dichlorophenyl)pyrimidine-5-carboxamide (250 mg, 0.742 mmol) was added at 0° C. The resulting reaction mixture was stirred at 80° C. for 1 h. After completion of the reaction (TLC and UPLC), the reaction mixture was diluted with water and extracted with EtOAc (200 mL×2). The combined organic layer was dried over Na₂SO₄ and concentrated under reduced pressure. The resulting crude material was purified by flash chromatography (SiO₂/100-200 mesh; 0-100% EtOAc/hexanes) to afford 2-chloro-4-cyclopropoxy-N-(2,6-dichlorophenyl)pyrimidine-5-carboxamide (76 mg, 28% yield) as white solid. Chemical Formula: C₁₄H₁₀Cl₃N₃O₂, Exact Mass: 356.98, Molecular Weight: 358.60. LCMS (ESI) m/z=360.0/361.0 (M+H) chlorine isotopes, t_{R}=2.210 min, 99.7% (Method-A).

### 4-cyclopropoxy-N-(2,6-dichlorophenyl)-2-((4-(4-methylpiperazin-1-yl)phenyl)amino) pyrimidine-5-carboxamide (Compound 102)

To a stirred solution of 2-chloro-4-cyclopropoxy-N-(2,6-dichlorophenyl)pyrimidine-5-carboxamide (70 mg, 0.195 mmol) in AcOH (2 mL) was added 4-(4-methylpiperazin-1-yl)aniline (37.3 mg, 0.195 mmol) at room temperature under nitrogen atmosphere. The resulting reaction mixture was subjected to microwave irradiation at 90° C. for 1 h. After completion of the reaction (TLC and UPLC), the reaction mixture was diluted with water and extracted with ethyl acetate (50 mL×2). The combined organic layer was dried over Na₂SO₄, filtered and concentrated under reduced pressure. The resulting crude material was purified by flash chromatography (SiO₂/100-200 mesh; 0-10% CH₃OH/CH₂Cl₂) to afford 4-cyclopropoxy-N-(2,6-dichlorophenyl)-2-((4-(4-methyl piperazin-1-yl)phenyl)amino)pyrimidine-5-carboxamide (Compound 102, 25 mg, 25% yield). ¹H-NMR (400 MHz, CD₃OD): δ 8.81 (s, 1H), 7.69 (d, J=6.4 Hz, 2H), 7.51 (d, J=8 Hz, 2H), 7.36-7.32 (m, 1H), 7.04-7.02 (m, 2H), 4.60 (br s, 1H), 3.30-3.27 (m, 4H), 2.93 (br s, 4H), 2.58 (s, 3H), 1.00-0.94 (m, 4H). (Note: Exchangeable protons were not observed). Chemical Formula: C₂₅H₂₆Cl₂N₆O₂, Exact Mass: 512.15, Molecular Weight: 513.42. LCMS (ESI) m/z=513.0 (M+H), t_{R}=1.556 min, 99.6% (Method-A); HPLC: 98.7%; t_{R}: 4.006 min.

### Example 4. Synthesis of N-(2-chlorophenyl)-4-methoxy-2-((3-methyl-4-(1-methylpiperidin-4-yl)phenyl)amino)pyrimidine-5-carboxamide (Compound 109)

### 2-chloro-N-(2-chlorophenyl)-4-methoxypyrimidine-5-carboxamide

To a stirred solution of 2,4-dichloro-N-(2-chlorophenyl)pyrimidine-5-carboxamide (300 mg, 0.99 mmol) in THE (5 mL) at 0° C. was added sodium methanolate (64.3 mg, 1.19 mmol), and allowed to stir at rt for 16 h. After completion of the reaction (TLC and UPLC), the mixture was quenched with water and extracted with EtOAc (60 mL×2). The combined organic layer was dried over Na₂SO₄, filtered and concentrated under reduced pressure to afford 2-chloro-N-(2-chlorophenyl)-4-methoxypyrimidine-5-carboxamide (290 mg, 98% yield) as a light-yellow solid. ¹H-NMR (400 MHz, DMSO-d₆): δ 10.04 (s, 1H), 8.95 (s, 1H), 8.20 (d, J=8.00 Hz, 1H), 7.60-7.57 (m, 1H), 7.43-7.39 (m, 1H), 7.27-7.22 (m, 1H), 4.17 (s, 3H). Chemical Formula: C₁₂H₉Cl₂N₃O₂, Exact Mass: 297.01, Molecular Weight: 298.12. LCMS (ESI) m/z=298.0/300.0 (M+H), chlorine isotopes, t_{R}: 2.133 min, 87.7% (Method-A).

### N-(2-chlorophenyl)-4-methoxy-2-((3-methyl-4-(1-methylpiperidin-4-yl)phenyl)amino)pyrimidine-5-carboxamide (Compound 109)

To a stirred solution of 2-chloro-N-(2-chlorophenyl)-4-methoxypyrimidine-5-carboxamide (150 mg, 0.50 mmol) in acetic acid (3 mL) was added 3-methyl-4-(1-methylpiperidin-4-yl)aniline (103 mg, 0.50 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~5% MeOH/CH₂Cl₂) to afford N-(2-chlorophenyl)-4-methoxy-2-((3-methyl-4-(1-methylpiperidin-4-yl)phenyl)amino)pyrimidine-5-carboxamide (Compound 109, 40 mg, 17% yield) as an off-white solid. ¹H-NMR (400 MHz, DMSO-d₆): δ 10.1 (br s, 1H), 9.96 (s, 1H), 8.87 (s, 1H), 8.46 (dd, J=8.4, 1.6 Hz, 1H), 7.59-7.56 (m, 3H), 7.37 (t, J=7.2 Hz, 1H), 7.19-7.14 (m, 2H), 4.20 (s, 3H), 2.96-2.89 (m, 2H), 2.34-2.30 (m, 6H), 2.20-2.11 (m, 1H), 1.70-1.60 (m, 4H), 1.25-1.24 (m, 2H). Chemical Formula: C₂₅H₂₈ClN₅O₂, Exact Mass: 465.19, Molecular Weight: 465.98. LCMS (ESI) m/z=466.2/468.2 (M+H), chlorine isotopes, t_{R}: 1.629 min, 98% (Method-A). HPLC: 98.6%; t_{R}: 4.372 min (Method-A).

### Example 5. Synthesis of N-(2-bromo-6-chlorophenyl)-4-methoxy-2-((3-methyl-4-(1-methyl piperidin-4-yl)phenyl)amino)pyrimidine-5-carboxamide (Compound 110)

### N-(2-bromo-6-chlorophenyl)-2-chloro-4-methoxypyrimidine-5-carboxamide

To a stirred solution of N-(2-bromo-6-chlorophenyl)-2,4-dichloropyrimidine-5-carboxamide (120 mg, 0.32 mmol) in THF (10 mL) at 0° C. was added sodium methanolate (17 mg, 0.32 mmol), and allowed to stir at rt for 16 h. After completion of the reaction (TLC and UPLC), the mixture was quenched with water and extracted with EtOAc (40 mL×2). The combined organic layer was dried over Na₂SO₄, filtered and concentrated under reduced pressure. The resulting crude material was purified by flash chromatography (SiO₂/100-200 mesh; ~10% EtOAc/hexanes) to afford N-(2-bromo-6-chlorophenyl)-2-chloro-4-methoxypyrimidine-5-carboxamide (102 mg, 86% yield) as a brown solid. ¹H-NMR (400 MHz, DMSO-d₆): δ 10.21 (s, 1H), 8.81 (s, 1H), 7.76-7.74 (m, 1H), 7.65-7.63 (m, 1H), 7.34 (t, J=8.4 Hz, 1H), 4.10 (s, 3H).

### N-(2-bromo-6-chlorophenyl)-4-methoxy-2-((3-methyl-4-(1-methyl piperidin-4-yl)phenyl)amino)pyrimidine-5-carboxamide (Compound 110)

To a stirred solution of N-(2-bromo-6-chlorophenyl)-2-chloro-4-methoxypyrimidine-5-carboxamide (102 mg, 0.27 mmol) in acetic acid (3 mL) was added 3-methyl-4-(1-methylpiperidin-4-yl)aniline (55 mg, 0.27 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~5% MeOH/CH₂Cl₂) to afford N-(2-bromo-6-chlorophenyl)-4-methoxy-2-((3-methyl-4-(1-methylpiperidin-4-yl)phenyl)amino)pyrimidine-5-carboxamide (Compound 110, 14 mg, 9% yield) as an off-white solid. ¹H-NMR (400 MHz, DMSO-d₆): δ 10.02 (s, 1H), 9.57 (s, 1H), 8.75 (s, 1H), 7.74-7.71 (m, 1H), 7.62-7.55 (m, 3H), 7.31 (t, J=8.4 Hz, 1H), 7.16 (d, J=8.4 Hz, 1H), 4.12 (s, 3H), 2.92-2.90 (m, 1H), 2.68-2.60 (m, 1H), 2.34-2.23 (m, 6H), 2.09-1.98 (m, 3H), 1.66-1.65 (m, 4H). Chemical Formula: C₂₅H₂₇BrClN₅O₂, Exact Mass: 543.10, Molecular Weight: 544.88. LCMS (ESI) m/z=544.0/546.0 (M+H), bromine isotopes, t_{R}: 1.563 min, 96.4% (Method-A). HPLC: 97.6%; t_{R}: 3.906 min (Method-A).

### Example 6. Synthesis of N-(2-bromo-6-methylphenyl)-4-methoxy-2-((3-methyl-4-(1-methylpiperidin-4-yl)phenyl)amino)pyrimidine-5-carboxamide (Compound 108)

### N-(2-bromo-6-methylphenyl)-2-chloro-4-methoxypyrimidine-5-carboxamide

To a stirred solution of N-(2-bromo-6-methylphenyl)-2,4-dichloropyrimidine-5-carboxamide (250 mg, 0.69 mmol) in THF (10 mL) at 0° C. was added sodium methanolate (45 mg, 0.83 mmol), and allowed to stir at rt for 24 h. After completion of the reaction (TLC and UPLC), the mixture was quenched with water and extracted with EtOAc (40 mL×2). The combined organic layer was dried over Na₂SO₄, filtered and concentrated under reduced pressure. The resulting crude material was purified by flash chromatography (SiO₂/100-200 mesh; ~10% EtOAc/hexanes) to afford N-(2-bromo-6-methylphenyl)-2-chloro-4-methoxypyrimidine-5-carboxamide (140 mg, 50% yield) as an off-white solid. ¹H-NMR (400 MHz, DMSO-d₆): δ 9.96 (s, 1H), 8.80 (s, 1H), 7.57 (d, J=8.0 Hz, 1H), 7.34 (d, J=7.2 Hz, 1H), 7.21 (t, J=7.6 Hz, 1H), 4.10 (s, 3H), 2.27 (s, 3H). Chemical Formula: C₁₃H₁₁BrClN₃O₂, Exact Mass: 354.97, Molecular Weight: 356.60. LCMS (ESI) m/z=356.0/358.0 (M+H), bromine isotopes, t_{R}: 1.925 min, 87.7% (Method-A).

### N-(2-bromo-6-methylphenyl)-4-methoxy-2-((3-methyl-4-(1-methylpiperidin-4-yl)phenyl)amino)pyrimidine-5-carboxamide (Compound 108)

To a stirred solution of N-(2-bromo-6-methylphenyl)-2-chloro-4-methoxypyrimidine-5-carboxamide (100 mg, 0.28 mmol) in acetic acid (3 mL) was added 3-methyl-4-(1-methylpiperidin-4-yl)aniline (63 mg, 0.31 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~10% MeOH/CH₂Cl₂) to afford N-(2-bromo-6-methylphenyl)-4-methoxy-2-((3-methyl-4-(1-methylpiperidin-4-yl)phenyl)amino)pyrimidine-5-carboxamide (Compound 108, 46 mg, 30% yield) as an off-white solid. ¹H-NMR (400 MHz, DMSO-d₆): δ 10.03 (s, 1H), 9.40 (s, 1H), 8.74 (s, 1H), 7.63-7.61 (m, 2H), 7.55 (d, J=8.0 Hz, 1H), 7.32 (d, J=6.8 Hz, 1H), 7.20-7.13 (m, 2H), 4.12 (s, 3H), 3.50-3.35 (m, 2H), 3.18-3.17 (m, 1H), 3.00-2.85 (m, 2H), 2.68 (s, 3H), 2.30 (s, 3H), 2.25 (s, 3H), 1.92-1.75 (m, 4H). Chemical Formula: C₂₆H₃₀BrN₅O₂, Exact Mass: 523.16, Molecular Weight: 524.46. LCMS (ESI) m/z=524.1/526.1 (M+H), bromine isotopes, t_{R}:1.526 min, 97.8% (Method-A). HPLC: 96.3%; t_{R}: 3.944 min (Method-A).

### Example 7. Synthesis of N-(2-cyanophenyl)-4-methoxy-2-((3-methyl-4-(1-methylpiperidin-4-yl)phenyl)amino)pyrimidine-5-carboxamide (Compound 115)

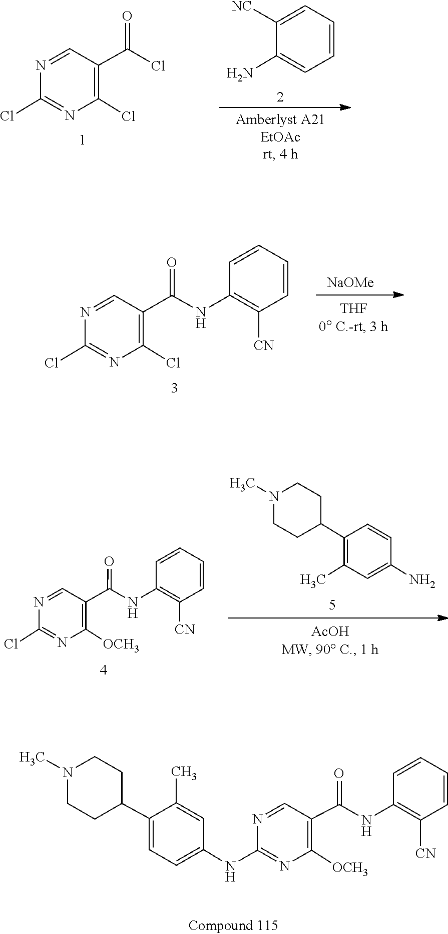

### 2,4-dichloro-N-(2-cyanophenyl)pyrimidine-5-carboxamide

To a stirred solution of 2,4-dichloropyrimidine-5-carbonyl chloride (100 mg, 0.47 mmol) in EtOAc (5 mL) was added aminobenzonitrile (55.9 mg, 0.47 mmol) followed by Amberlyst A21 (20 mg) at room temperature under nitrogen atmosphere and allowed to stir for 4 h. After completion of the reaction (TLC and UPLC), the mixture was filtered through sintered funnel and the filtrate was diluted with saturated aq. NaHCO₃ solution and extracted with EtOAc (50 mL×2). The combined organic layer was dried over Na₂SO₄, filtered and concentrated under reduced pressure. The resulting crude material was purified by flash chromatography (SiO₂/230-400 mesh; ~25% EtOAc/hexanes) to afford 2,4-dichloro-N-(2-cyanophenyl)pyrimidine-5-carboxamide (60 mg, 42% yield) as an off-white solid. ¹H-NMR (400 MHz, DMSO-d₆): δ 11.07 (s, 1H), 9.11 (s, 1H), 7.93 (d, J=7.60 Hz, 1H), 7.81-7.79 (m, 2H), 7.50-7.46 (m, 1H). Chemical Formula: C₁₂H₆Cl₂N₄O, Exact Mass: 291.99, Molecular Weight: 293.11. LCMS (ESI) m/z=291.0/293.0 (M−H), chlorine isotopes, t_{R}: 1.640 min, 97.7% (Method-A).

### 2-chloro-N-(2-cyanophenyl)-4-methoxypyrimidine-5-carboxamide

To a stirred solution of 2,4-dichloro-N-(2-cyanophenyl)pyrimidine-5-carboxamide (60 mg, 0.21 mmol) in THE (5 mL) at 0° C. was added sodium methanolate (13.3 mg, 0.25 mmol), and allowed to stir at rt for 3 h. After completion of the reaction (TLC and UPLC), the mixture was quenched with water and extracted with EtOAc (50 mL×2). The combined organic layer was dried over Na₂SO₄, filtered and concentrated under reduced pressure. The resulting crude material was purified by flash chromatography (SiO₂/230-400 mesh; ~25% EtOAc/hexanes) to afford 2-chloro-N-(2-cyanophenyl)-4-methoxypyrimidine-5-carboxamide (70 mg, 73% yield) as an off-white solid. ¹H-NMR (400 MHz, DMSO-d₆): δ 10.40 (s, 1H), 8.91 (s, 1H), 8.04 (d, J=8.4 Hz, 1H), 7.91 (dd, J=8.4, 1.6 Hz, 1H), 7.80-7.75 (m, 1H), 7.41 (td, J=8.0, 1.2 Hz, 1H), 4.14 (s, 3H). Chemical Formula: C₁₃H₉ClN₄O₂, Exact Mass: 288.04, Molecular Weight: 288.69. LCMS (ESI) m/z=291.0/293.0 (M−H), chlorine isotopes, t_{R}: 2.024 min, 61.7% (Method-A).

### N-(2-cyanophenyl)-4-methoxy-2-((3-methyl-4-(1-methylpiperidin-4-yl)phenyl)amino)pyrimidine-5-carboxamide (Compound 115)

To a stirred solution of 2-chloro-N-(2-cyanophenyl)-4-methoxypyrimidine-5-carboxamide (60 mg, 0.21 mmol) in acetic acid (3 mL) was added 3-methyl-4-(1-methylpiperidin-4-yl)aniline (47 mg, 0.23 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~10% MeOH/CH₂Cl₂) to afford N-(2-cyanophenyl)-4-methoxy-2-((3-methyl-4-(1-methylpiperidin-4-yl)phenyl)amino)pyrimidine-5-carboxamide (Compound 115, 28 mg, 29% yield) as an off-white solid. ¹H NMR (400 MHz, DMSO-d₆): δ 10.20 (s, 1H), 10.10 (s, 1H), 8.86 (s, 1H), 8.37 (d, J=8.4 Hz, 1H), 7.88 (d, J=6.8 Hz, 1H), 7.75 (t, J=7.6 Hz, 1H), 7.62-7.60 (m, 2H), 7.33 (t, J=8.0 Hz, 1H), 7.15-7.13 (m, 1H), 4.19 (s, 3H), 3.38-3.55 (m, 2H), 2.85-2.82 (m, 1H), 2.69-2.59 (m, 5H), 2.33 (s, 3H), 1.92-1.76 (m, 4H). Chemical Formula: C₂₆H₂₈N₆O₂, Exact Mass: 456.23, Molecular Weight: 456.55. LCMS (ESI) m/z=457.2 (M+H), t_{R}: 1.610 min, 99.2% (Method-A). HPLC: 98.9%; t_{R}: 4.078 min (Method-A).

### Example 8. Synthesis of 4-methoxy-2-((3-methyl-4-(1-methylpiperidin-4-yl)phenyl)amino)-N-(2-methyl-6-(trifluoromethyl)phenyl)pyrimidine-5-carboxamide (Compound 120)

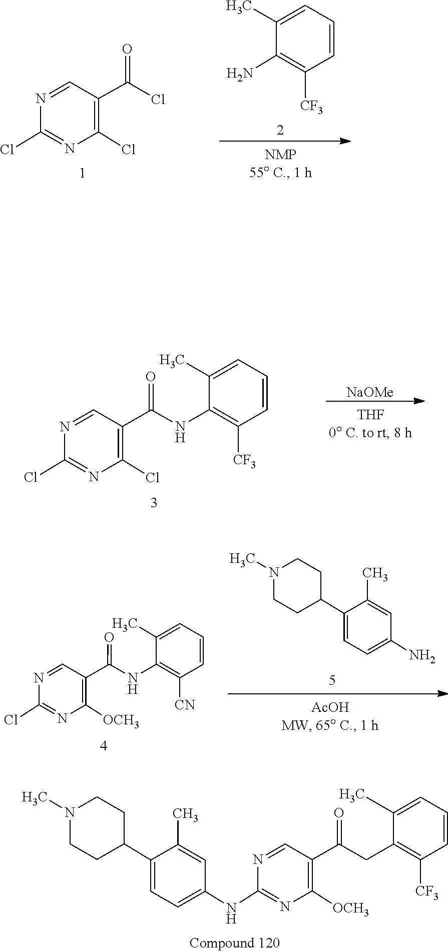

### 2,4-dichloro-N-(2-methyl-6-(trifluoromethyl)phenyl)pyrimidine-5-carboxamide

To a stirred solution of 2,4-dichloropyrimidine-5-carbonyl chloride (500 mg, 2.36 mmol) in NMP (5 mL) was added 2-methyl-6-(trifluoromethyl)aniline (414 mg, 2.36 mmol) at room temperature under nitrogen atmosphere, and allowed to stir at 55° C. for 1 h. After completion of the reaction (TLC and UPLC), the mixture was diluted with water and extracted with EtOAc (50 mL×2). The combined organic layer was dried over Na₂SO₄, filtered and concentrated under reduced pressure. The resulting crude material was purified by flash chromatography (SiO₂/60-120 mesh; ~13% EtOAc/hexanes) to afford 2,4-dichloro-N-(2-methyl-6-(trifluoromethyl)phenyl)pyrimidine-5-carboxamide (600 mg, 71% yield) as an off-white solid. ¹H-NMR (400 MHz, DMSO-d₆): δ 10.57 (s, 1H), 8.93 (s, 1H), 7.69 (t, J=8.4 Hz, 2H), 7.53 (t, J=7.6 Hz, 1H), 1.53 (s, 3H). Chemical Formula: Cl₃H₈Cl₂F₃N₃O, Exact Mass: 349.00, Molecular Weight: 350.12. LCMS (ESI) m/z=350.1/352.1 (M+H), chlorine isotopes, t_{R}: 2.005 min, 98.4% (Method-A).

### 2-chloro-4-methoxy-N-(2-methyl-6-(trifluoromethyl)phenyl)pyrimidine-5-carboxamide

To a stirred solution of 2,4-dichloro-N-(2-methyl-6-(trifluoromethyl)phenyl)pyrimidine-5-carboxamide (600 mg, 1.71 mmol) in THF (10 mL) at 0° C. was added sodium methanolate (111 mg, 2.05 mmol), and allowed to stir at rt for 8 h. After completion of the reaction (TLC and UPLC), the mixture was quenched with water and extracted with EtOAc (40 mL×2). The combined organic layer was dried over Na₂SO₄, filtered and concentrated under reduced pressure. The resulting crude material was purified by flash chromatography (SiO₂/100-200 mesh; ~10% EtOAc/hexanes) to afford 2-chloro-4-methoxy-N-(2-methyl-6-(trifluoromethyl)phenyl)pyrimidine-5-carboxamide (435 mg, 35% yield) as an off-white solid. ¹H-NMR (400 MHz, DMSO-d₆): δ 9.96 (s, 1H), 8.74 (s, 1H), 7.68-7.64 (m, 2H), 7.50 (t, J=7.6 Hz, 1H), 4.08 (s, 3H), 2.27 (s, 3H).

### 4-methoxy-2-((3-methyl-4-(1-methylpiperidin-4-yl)phenyl)amino)-N-(2-methyl-6-(trifluoromethyl)phenyl)pyrimidine-5-carboxamide (Compound 120)

To a stirred solution of 2-chloro-4-methoxy-N-(2-methyl-6-(trifluoromethyl)phenyl) pyrimidine-5-carboxamide (435 mg, 1.25 mmol) in acetic acid (10 mL) was added 3-methyl-4-(1-methylpiperidin-4-yl)aniline (309 mg, 1.51 mmol), and the resulting reaction mixture was subjected to microwave irradiation at 65° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~10% MeOH/CH₂Cl₂) to afford N-(2-chloro-6-fluorophenyl)-4-methoxy-2-((3-methyl-4-(1-methylpiperidin-4-yl)phenyl)amino)pyrimidine-5-carboxamide (Compound 120, 31 mg, 4% yield) as an off-white solid. ¹H-NMR (400 MHz, DMSO-d₆): δ 10.04 (s, 1H), 9.41 (s, 1H), 8.72 (s, 1H), 7.58-7.65 (m, 4H), 7.47 (t, J=7.60 Hz, 1H), 7.14 (d, J=8.80 Hz, 1H), 4.10 (s, 3H), 3.24 (s, 3H), 2.81 (s, 1H), 2.33 (s, 3H), 2.25 (s, 3H), 1.91-1.79 (m, 4H). Note: 4H merged under solvent peak. Chemical Formula: C₂₇H₃₀F₃N₅O₂, Exact Mass: 513.24, Molecular Weight: 513.56, LCMS (ESI) m/z=514.2 (M+H), t_{R}:1.597 min, 98.5% (Method-A). HPLC:98.1%; t_{R}: 4.075 min (Method-A).

### Example 9. Synthesis of N-(2-bromophenyl)-4-methoxy-2-((3-methyl-4-(1-methylpiperidin-4-yl)phenyl)amino)pyrimidine-5-carboxamide (Compound 107)

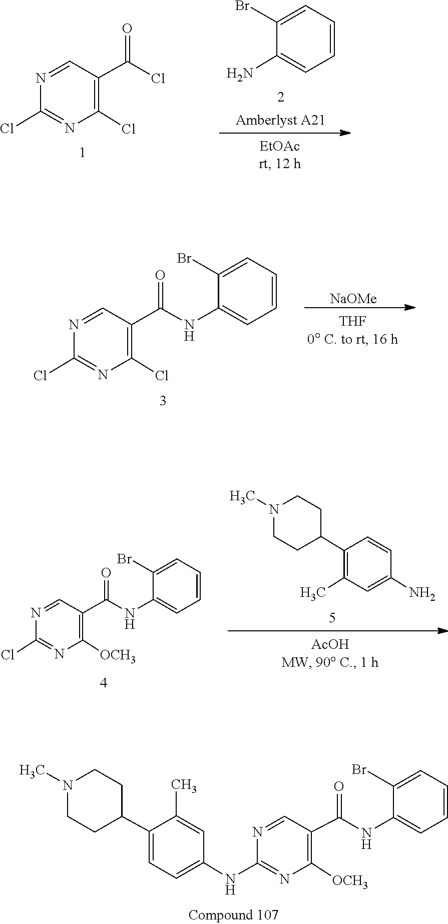

### N-(2-bromophenyl)-2,4-dichloropyrimidine-5-carboxamide

To a stirred solution of 2,4-dichloropyrimidine-5-carbonyl chloride (400 mg, 1.89 mmol) in EtOAc (15 mL) was added 2-bromoaniline (325 mg, 1.89 mmol) followed by Amberlyst A21 (80 mg) at room temperature under nitrogen atmosphere and allowed to stir for 16 h. After completion of the reaction (TLC and UPLC), the mixture was filtered through sintered funnel and the filtrate was diluted with saturated aq. NaHCO₃ solution and extracted with EtOAc (200 mL×3). The combined organic layer was dried over Na₂SO₄, filtered and concentrated under reduced pressure. The resulting crude material was purified by flash chromatography (SiO₂/60-120 mesh; ~15% EtOAc/hexanes) to afford N-(2-bromophenyl)-2,4dichloropyrimidine-5-carboxamide (380 mg, 53% yield) as a brown solid. ¹H-NMR (400 MHz, DMSO-d₆): δ 10.44 (s, 1H), 9.07 (s, 1H), 7.74-7.57 (m, 2H), 7.48 (t, J=7.2 Hz, 1H), 7.28-7.24 (m, 1H). Chemical Formula: C₁₁H₆BrCl₂N₃O, Exact Mass: 344.91, Molecular Weight: 346.99. LCMS (ESI) m/z=344.0/346.0 (M−H), bromine isotopes, t_{R}: 1.935 min, 92.3% (Method-A).

### N-(2-bromophenyl)-2-chloro-4-methoxypyrimidine-5-carboxamide

To a stirred solution of N-(2-bromophenyl)-2,4-dichloropyrimidine-5-carboxamide (380 mg, 1.09 mmol) in tetrahydrofuran (10 mL) at 0° C. was added sodium methanolate (77 mg, 1.42 mmol), and allowed to stir at rt for 16 h. After completion of the reaction (TLC and UPLC), the mixture was quenched with water and extracted with EtOAc (60 mL×2). The combined organic layer was dried over Na₂SO₄, filtered and concentrated under reduced pressure. The resulting crude material was purified by flash chromatography (SiO₂/100-200 mesh; ~12% EtOAc/hexanes) to afford N-(2-bromophenyl)-2-chloro-4-methoxypyrimidine-5-carboxamide (180 mg, 47% yield) as a brown solid. ¹H-NMR (400 MHz, DMSO-d₆): δ 10.00 (s, 1H), 8.97 (s, 1H), 8.21-8.19 (m, 1H), 7.76-7.73 (m, 1H), 7.45 (t, J=7.2 Hz, 1H), 7.20-7.15 (m, 1H), 4.20 (s, 3H). Chemical Formula: C₁₂H₉BrClN₃O₂, Exact Mass: 340.96, Molecular Weight: 342.57. LCMS (ESI) m/z=342.0/344.0 (M+H), bromine isotopes, t_{R}: 1.307 min, 82.4% (Method-A).

### N-(2-bromophenyl)-4-methoxy-2-((3-methyl-4-(1-methylpiperidin-4-yl)phenyl)amino)pyrimidine-5-carboxamide (Compound 107)

To a stirred solution of N-(2-bromophenyl)-2-chloro-4-methoxypyrimidine-5-carboxamide (130 mg, 0.38 mmol) in acetic acid (3 mL) was added 3-methyl-4-(1-methylpiperidin-4-yl)aniline (93 mg, 0.45 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~10% MeOH/CH₂Cl₂) to afford N-(2-bromophenyl)-4-methoxy-2-((3-methyl-4-(1-methylpiperidin-4-yl)phenyl)amino)pyrimidine-5-carboxamide (Compound 107, 55 mg, 28% yield) as an off-white solid. ¹H-NMR (400 MHz, DMSO-d₆): δ 10.16 (s, 1H), 9.93 (s, 1H), 8.89 (s, 1H), 8.48-8.45 (m, 1H), 7.73-7.71 (m, 1H), 7.61-7.59 (m, 2H), 7.44 (t, J=7.20 Hz, 1H), 7.10-7.07 (m, 2H), 4.23 (s, 3H), 3.32-3.27 (m, 2H), 2.68-2.67 (m, 1H), 2.53-2.50 (m, 2H), 2.50-2.32 (m, 4H), 1.82-1.79 (m, 4H). Note: 2H merged under solvent peak. Chemical Formula: C₂₅H₂₈BrN₅O₂, Exact Mass: 509.14, Molecular Weight: 510.44. LCMS (ESI) m/z=510.0/512.0 (M+H), bromine isotopes, t_{R}: 1.667 min, 99.3% (Method-A). HPLC: 99%; t_{R}: 4.393 min (Method-A).

### Example 10. Synthesis of N-(2-chloro-6-fluorophenyl)-4-methoxy-2-((3-methyl-4-(1-methylpiperidin-4-yl)phenyl)amino)pyrimidine-5-carboxamide (Compound 113)

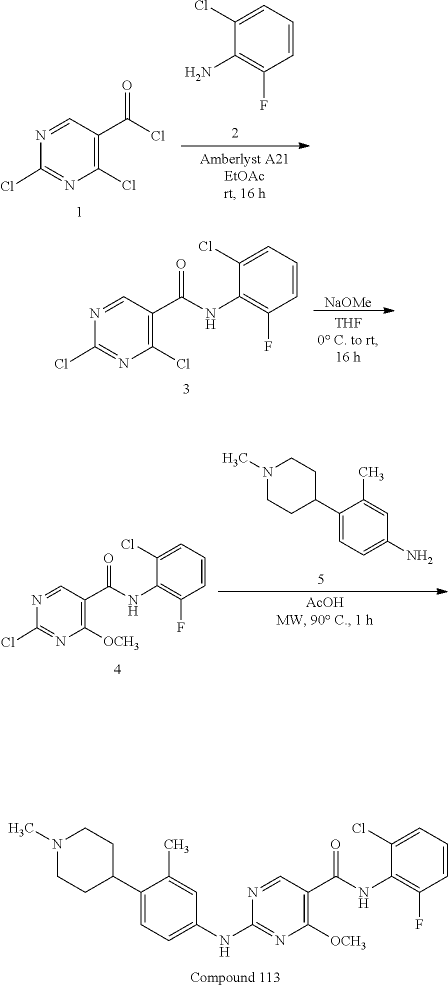

### 2,4-dichloro-N-(2-chloro-6-fluorophenyl)pyrimidine-5-carboxamide

To a stirred solution of 2,4-dichloropyrimidine-5-carbonyl chloride (350 mg, 1.65 mmol) in EtOAc (15 mL) was added 2-chloro-6-fluoroaniline (265 mg, 1.82 mmol) followed by Amberlyst A21 (70 mg) at room temperature under nitrogen atmosphere and allowed to stir for 16 h. After completion of the reaction (TLC and UPLC), the mixture was diluted with water and extracted with EtOAc (50 mL×2). The combined organic layer was dried over Na₂SO₄, filtered and concentrated under reduced pressure. The resulting crude material was purified by flash chromatography (SiO₂/60-120 mesh; ~8% EtOAc/hexanes) to afford 2,4-dichloro-N-(2-chloro-6-fluorophenyl)pyrimidine-5-carboxamide (170 mg, 29% yield) as an off-white solid. ¹H-NMR (400 MHz, DMSO-d₆): δ 10.71 (s, 1H), 9.04 (s, 1H), 7.48-7.39 (m, 3H). Chemical Formula: C₁₁H₅Cl₃FN₃O, Exact Mass: 318.95, Molecular Weight: 320.53. LCMS (ESI) m/z=320.0/322.0 (M+H), chlorine isotopes, t_{R}: 1.775 min, 99.4% (Method-A).

### 2-chloro-N-(2-chloro-6-fluorophenyl)-4-methoxypyrimidine-5-carboxamide

To a stirred solution of 2,4-dichloro-N-(2-chloro-6-fluorophenyl)pyrimidine-5-carboxamide (170 mg, 0.53 mmol) in THF (10 mL) at 0° C. was added sodium methanolate (34.4 mg, 0.64 mmol), and allowed to stir at rt for 16 h. After completion of the reaction (TLC and UPLC), the mixture was quenched with water and extracted with EtOAc (40 mL×2). The combined organic layer was dried over Na₂SO₄, filtered and concentrated under reduced pressure. The resulting crude material was purified by flash chromatography (SiO₂/100-200 mesh; ~10% EtOAc/hexanes) to afford 2-chloro-N-(2-chloro-6-fluorophenyl)-4-methoxypyrimidine-5-carboxamide (130 mg, 90% yield) as an off-white solid. ¹H-NMR (400 MHz, DMSO-d₆): δ 10.03 (s, 1H), 8.82 (s, 1H), 7.47-7.43 (m, 2H), 7.39-7.34 (m, 1H), 4.10 (s, 3H).

### N-(2-chloro-6-fluorophenyl)-4-methoxy-2-((3-methyl-4-(1-methylpiperidin-4-yl)phenyl)amino)pyrimidine-5-carboxamide (Compound 113)

To a stirred solution of 2-chloro-N-(2-chloro-6-fluorophenyl)-4-methoxypyrimidine-5-carboxamide (50 mg, 0.15 mmol) in acetic acid (2 mL) was added 3-methyl-4-(1-methylpiperidin-4-yl)aniline (32 mg, 0.15 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~10% MeOH/CH₂Cl₂) to afford N-(2-chloro-6-fluorophenyl)-4-methoxy-2-((3-methyl-4-(1-methylpiperidin-4-yl)phenyl)amino)pyrimidine-5-carboxamide (Compound 113, 8 mg, 10% yield) as an off-white solid. ¹H-NMR (400 MHz, DMSO-d₆): δ 10.02 (s, 1H), 9.41 (s, 1H), 8.74 (s, 1H), 7.57-7.55 (m, 2H), 7.45-7.40 (m, 3H), 7.36-7.31 (m, 1H), 4.11 (s, 3H), 2.91-2.86 (m, 2H), 2.68-2.67 (m, 3H), 2.29 (s, 3H), 2.20 (s, 3H), 1.65-1.64 (m, 4H). Chemical Formula: C₂₅H₂₇ClFN₅O₂, Exact Mass: 483.18, Molecular Weight: 483.97. LCMS (ESI) m/z=484.1/486.1 (M+H), chlorine isotopes, t_{R}: 1.824 min, 97.2% (Method-A). HPLC: 99.0%; t_{R}: 3.806 min (Method-A).

### Example 11. Synthesis of N-(2-ethynylphenyl)-4-methoxy-2-((3-methyl-4-(1-methylpiperidin-4-yl)phenyl)amino)pyrimidine-5-carboxamide (Compound 126)

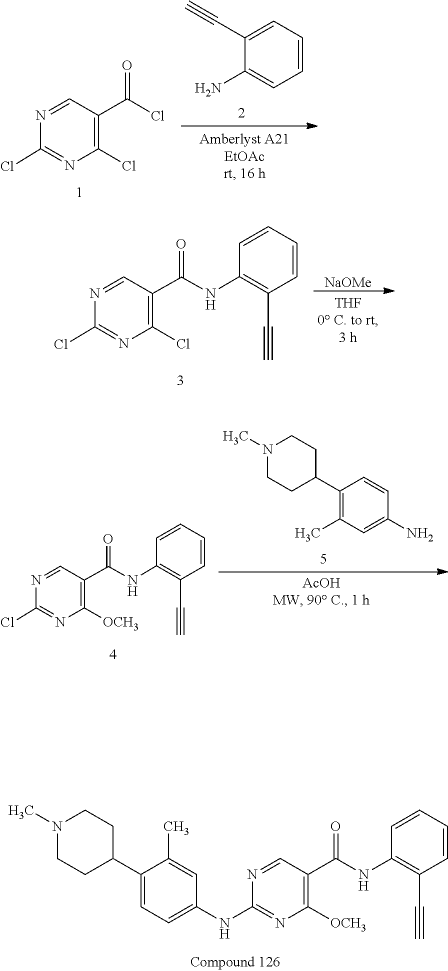

### 2,4-dichloro-N-(2-ethynylphenyl)pyrimidine-5-carboxamide

To a stirred solution of 2,4-dichloropyrimidine-5-carbonyl chloride (500 mg, 2.36 mmol) in EtOAc (30 mL) was added 2-ethynylaniline (277 mg, 2.36 mmol) followed by Amberlyst A21 (100 mg) at room temperature under nitrogen atmosphere and allowed to stir for 16 h. After completion of the reaction (TLC and UPLC), the mixture was filtered through sintered funnel and the filtrate was diluted with saturated aq. NaHCO₃ solution and extracted with EtOAc (200 mL×3). The combined organic layer was dried over Na₂SO₄, filtered and concentrated under reduced pressure. The resulting crude material was purified by flash chromatography (SiO₂/100-200 mesh; ~6% EtOAc/hexanes) to afford 2,4-dichloro-N-(2-ethynylphenyl)pyrimidine-5-carboxamide (600 mg, 51% yield) as an off-white solid. Chemical Formula: C₁₃H₇Cl₂N₃O, Exact Mass: 291.00, Molecular Weight: 292.12. LCMS (ESI) m/z=292.0 (M+H), t_{R}: 1.664 min, 51% (Method-A).

### 2-chloro-N-(2-ethynylphenyl)-4-methoxypyrimidine-5-carboxamide

To a stirred solution of 2,4-dichloro-N-(2-ethynylphenyl)pyrimidine-5-carboxamide (500 mg, 1.71 mmol) in THF (10 mL) at 0° C. was added sodium methanolate (111 mg, 2.05 mmol), and allowed to stir at rt for 3 h. After completion of the reaction (TLC and UPLC), the mixture was quenched with water and extracted with EtOAc (60 mL×2). The combined organic layer was dried over Na₂SO₄, filtered and concentrated under reduced pressure. The resulting crude material was purified by flash chromatography (SiO₂/100-200 mesh; ~10% EtOAc/hexanes) to afford 2-chloro-N-(2-ethynylphenyl)-4-methoxypyrimidine-5-carboxamide (180 mg, 36% yield) as an off-white solid. ¹H-NMR (400 MHz, DMSO-d₆): δ 10.11 (s, 1H), 9.03 (s, 1H), 8.45 (d, J=8.4 Hz, 1H), 7.59-7.57 (m, 2H), 7.21-7.17 (m, 1H), 4.98 (s, 1H), 4.21 (s, 3H). Chemical Formula: C₁₄H₁₀ClN₃O₂, Exact Mass: 287.05, Molecular Weight: 287.70. LCMS (ESI) m/z=288.0/290.2 (M+H)/Cl-isotopes, t_{R}: 2.086 min, 85% (Method-A).

### N-(2-ethynylphenyl)-4-methoxy-2-((3-methyl-4-(1-methylpiperidin-4-yl)phenyl)amino)pyrimidine-5-carboxamide (Compound 126)

To a stirred solution of 2-chloro-N-(2-ethynylphenyl)-4-methoxypyrimidine-5-carboxamide (100 mg, 0.35 mmol) in acetic acid (3 mL) was added 3-methyl-4-(1-methylpiperidin-4-yl)aniline (71.0 mg, 0.35 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~10% MeOH/CH₂Cl₂) to afford N-(2-ethynylphenyl)-4-methoxy-2-((3-methyl-4-(1-methylpiperidin-4-yl)phenyl)amino)pyrimidine-5-carboxamide (DS19-TM23, 10 mg, 6% yield). ¹H-NMR (400 MHz, DMSO-d₆): δ 10.14 (br s, 1H), 10.1 (s, 1H), 8.90 (s, 1H), 8.57 (d, J=8.4 Hz, 1H), 7.59-7.54 (m, 3H), 7.45 (t, J=8.4 Hz, 1H), 7.18-7.11 (m, 2H), 4.98 (s, 1H), 4.21 (s, 3H), 3.08-3.06 (m, 2H), 2.71-2.68 (m, 1H), 2.39-2.36 (m, 3H), 2.31 (s, 3H), 1.73-1.62 (m, 3H), 1.25-1.15 (m, 3H). Chemical Formula: C₂₇H₂₉N₅O₂, Exact Mass: 455.23, Molecular Weight: 455.56. LCMS (ESI) m/z=456.2 (M+H), t_{R}: 1.679 min, 99.6% (Method-A). HPLC: 98.9%; t_{R}: 6.243 min (Method-C).

### Example 12. Synthesis of N-(2-chloro-6-methylphenyl)-4-methoxy-2-((3-methyl-4-(1-methylpiperidin-4-yl)phenyl)amino)pyrimidine-5-carboxamide (Compound 106)

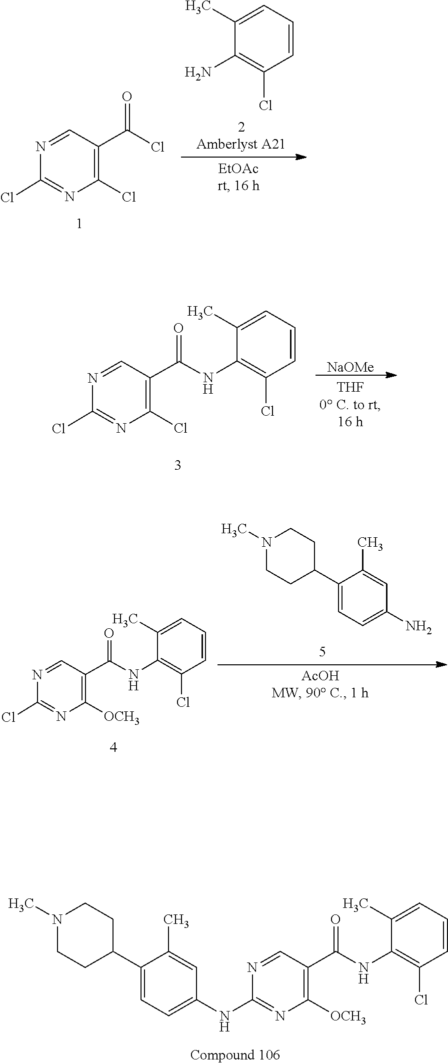

### 2,4-dichloro-N-(2-chloro-6-methylphenyl)pyrimidine-5-carboxamide

To a stirred solution of 2,4-dichloropyrimidine-5-carbonyl chloride (500 mg, 2.36 mmol) in EtOAc (40 mL) was added 2-chloro-6-methylaniline (335 mg, 2.36 mmol) followed by Amberlyst A 21 (100 mg) at room temperature under nitrogen atmosphere and allowed to stir for 16 h. After completion of the reaction (TLC and UPLC), the mixture was filtered through sintered funnel and the filtrate was diluted with saturated aq. NaHCO₃ solution and extracted with EtOAc (200 mL×3). The combined organic layer was dried over Na₂SO₄, filtered and concentrated under reduced pressure. The resulting crude material was washed with MTBE to afford 2,4-dichloro-N-(2-chloro-6-methylphenyl)pyrimidine-5-carboxamide (600 mg, 80% yield) as an off-white solid. ¹H-NMR (400 MHz, DMSO-d₆): δ 10.52 (s, 1H), 9.06 (s, 1H), 7.45-7.42 (m, 1H), 7.32-7.30 (m, 2H), 2.33 (s, 3H). Chemical Formula: C₁₂H₈Cl₃N₃O, Exact Mass: 314.97, Molecular Weight: 316.57. LCMS (ESI) m/z=316.0/318.0 (M+H), chlorine isotopes, t_{R}: 1.835 min, 90.2% (Method-A).

### 2-chloro-N-(2-chloro-6-methylphenyl)-4-methoxypyrimidine-5-carboxamide

To a stirred solution of 2,4-dichloro-N-(2-chloro-6-methylphenyl)pyrimidine-5-carboxamide (400 mg, 1.26 mmol) in tetrahydrofuran (10 mL) at 0° C. was added sodium methanolate (71.7 mg, 1.33 mmol), and allowed to stir at rt for 16 h. After completion of the reaction (TLC and UPLC), the mixture was quenched with water and extracted with EtOAc (60 mL×2). The combined organic layer was dried over Na₂SO₄, filtered and concentrated under reduced pressure. The resulting crude material was purified by flash chromatography (SiO₂/100-200 mesh; ~12% EtOAc/hexanes) to afford 2-chloro-N-(2-chloro-6-methylphenyl)-4-methoxypyrimidine-5-carboxamide (100 mg, 25% yield) as an off-white solid. ¹H-NMR (400 MHz, DMSO-d₆): δ 9.93 (s, 1H), 8.81 (s, 1H), 7.42-7.40 (m, 1H), 7.32-7.26 (m, 2H), 4.10 (s, 3H), 2.26 (s, 3H). Chemical Formula: C₁₃H₁₁Cl₂N₃O₂, Exact Mass: 311.02, Molecular Weight: 312.15. LCMS (ESI) m/z=312.0/314.0 (M+H), chlorine isotopes, t_{R}: 1.902 min, 99.8% (Method-A).

### N-(2-chloro-6-methylphenyl)-4-methoxy-2-((3-methyl-4-(1-methylpiperidin-4-yl)phenyl)amino)pyrimidine-5-carboxamide (Compound 106)

To a stirred solution of 2-chloro-N-(2-chloro-6-methylphenyl)-4-methoxypyrimidine-5-carboxamide (100 mg, 0.32 mmol) in acetic acid (3.0 mL) was added 3-methyl-4-(1-methylpiperidin-4-yl)aniline (65.5 mg, 0.32 mmol), and the resulting reaction mixture was subjected to microwave irradiation at 90° C. for 1 h. After completion of the reaction (TLC and UPLC), the volatiles were removed under reduced pressure to get the crude residue and neutralized with saturated aq. NaHCO₃ solution (pH=9). The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~10% MeOH/CH₂Cl₂) to afford N-(2-chloro-6-methylphenyl)-4-methoxy-2-((3-methyl-4-(1-methylpiperidin-4-yl)phenyl)amino)pyrimidine-5-carboxamide (Compound 106, 60 mg, 39% yield) as an off-white solid. ¹H-NMR (400 MHz, DMSO-d₆): δ 10.02 (s, 1H), 9.38 (s, 1H), 8.74 (s, 1H), 7.62-7.60 (m, 2H), 7.39 (dd, J=7.2, 1.6 Hz, 1H), 7.30-7.23 (m, 2H), 7.13 (d, J=8.4 Hz, 1H), 4.11 (s, 3H), 2.91-2.89 (m, 3H), 2.67-2.59 (m, 3H), 2.32 (s, 3H), 2.24 (s, 3H), 1.91-1.81 (m, 4H) Note: 2H merged under solvent peak. HPLC: 98.5%; t_{R}: 3.946 min (Method-A). Chemical Formula: C₂₆H₃₀ClN₅O₂, Exact Mass: 479.21, Molecular Weight: 480.01. LCMS (ESI) m/z=480.2 (M+H), t_{R}: 1.574 min, 99.6% (Method-A).

### Example 13. Synthesis of N-(2,6-dichlorophenyl)-4-(2-hydroxyethoxy)-2-((4-(4-methylpiperazin-1-yl)phenyl)amino)pyrimidine-5-carboxamide (Compound 123

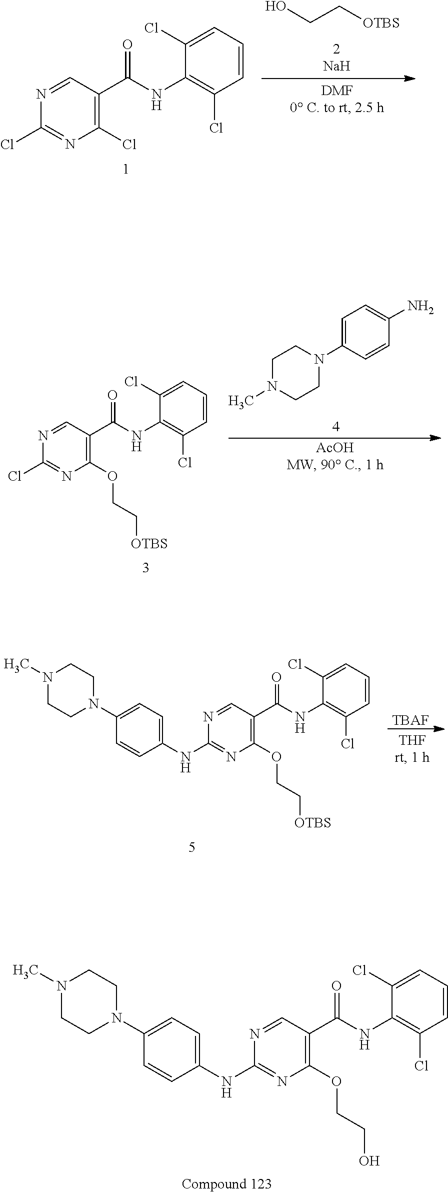

### 4-(2-((tert-butyldimethylsilyl)oxy)ethoxy)-2-chloro-N-(2,6-dichlorophenyl)pyrimidine-5-carboxamide

To a stirred solution of 2-((tert-butyldimethylsilyl)oxy)ethan-1-ol (700 mg, 4.89 mmol) in DMF (15 ml) at 0° C. was added sodium hydride (862 mg, 5.34 mmol) and the reaction mixture was stirred for 30 min. To this solution 2,4-dichloro-N-(2,6-dichlorophenyl)pyrimidine-5-carboxamide (1.5 g, 4.45 mmol) was added at 0° C. The resulting reaction mixture was stirred at rt for 2 h. After completion of the reaction (TLC and UPLC), the mixture was quenched with saturated aq. NH₄C₁ solution and extracted with EtOAc (100 mL×2). The combined organic layer was dried over Na₂SO₄, filtered and concentrated under reduced pressure. The resulting crude material was purified by flash chromatography (SiO₂/230-400 mesh; ~12% EtOAc/hexanes) to afford 4-(2-((tert-butyldimethylsilyl)oxy)ethoxy)-2-chloro-N-(2,6-dichlorophenyl)pyrimidine-5-carboxamide (1.1 g, 35% yield) as an off-white solid. Chemical Formula: C₁₉H₂₄Cl₃N₃O₃Si, Exact Mass: 475.07, Molecular Weight: 476.85. LCMS (ESI) ni/z=476.2/478.2 (M+H), chlorine isotopes, t_{R}: 2.701 min, 79% (Method-A).

### 4-(2-((tert-butyldimethylsilyl)oxy)ethoxy)-N-(2,6-dichlorophenyl)-2-((4-(4-methylpiperazin-1-yl)phenyl)amino)pyrimidine-5-carboxamide

To a stirred solution of 4-(2-((tert-butyldimethylsilyl)oxy)ethoxy)-2-chloro-N-(2,6-dichlorophenyl)pyrimidine-5-carboxamide (1.1 g, 2.31 mmol) in acetic acid (10 mL) was added 4-(4-methylpiperazin-1-yl)aniline (440 mg, 2.31 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (30 mL×3), and the combined organic layer was dried over Na₂SO₄, filtered and concentrated under reduced pressure. The resulting crude material was purified by flash chromatography (SiO₂/230-400 mesh; ~10% MeOH/CH₂Cl₂) to afford 4-(2-((tert-butyldimethylsilyl)oxy)ethoxy)-N-(2,6-dichlorophenyl)-2-((4-(4-methylpiperazin-1-yl)phenyl)amino)pyrimidine-5-carboxamide (700 mg, 48% yield) as an off-white solid. Chemical Formula: C₃OH₄OCl₂N₆O₃Si, Exact Mass: 630.23, Molecular Weight: 631.67. LCMS (ESI) m/z=631.2/633.2 (M+H), chlorine isotopes, t_{R}: 1.99 min, 86.5% (Method-A)

### N-(2,6-dichlorophenyl)-4-(2-hydroxyethoxy)-2-((4-(4-methylpiperazin-1-yl)phenyl)amino)pyrimidine-5-carboxamide (Compound 123)

To a stirred solution of 4-(2-((tert-butyldimethylsilyl)oxy)ethoxy)-N-(2,6-dichlorophenyl)-2-((4-(4-methylpiperazin-1-yl)phenyl)amino)pyrimidine-5-carboxamide (700 mg, 1.11 mmol) in THF (10 ml) at 0° C. was added TBAF (1M in THF) (1.67 mL, 1.67 mmol), and allowed to stir at rt for 1 h. After completion of the reaction (TLC and UPLC), the volatiles were removed under reduced pressure and the crude residue was diluted with saturated aq. NH₄Cl solution. The aqueous layer was extracted with CH₂Cl₂ (30 mL×3), and the combined organic layer was dried over Na₂SO₄, filtered and concentrated under reduced pressure. The resulting crude material was purified by flash chromatography (SiO₂/230-400 mesh; ~12% MeOH/CH₂Cl₂) to afford N-(2,6-dichlorophenyl)-4-(2-hydroxyethoxy)-2-((4-(4-methylpiperazin-1-yl)phenyl)amino)pyrimidine-5-carboxamide (Compound 123, 320 mg, 56% yield) as an off-white solid. ¹H-NMR (400 MHz, DMSO-d₆): δ 9.97 (s, 1H), 9.36 (s, 1H), 8.74 (s, 1H), 7.56-7.60 (m, 4H), 7.39 (t, J=8.40 Hz, 1H), 6.94 (d, J=9.20 Hz, 2H), 5.05 (t, J=5.6 Hz, 1H), 4.54 (t, J=5.2 Hz, 2H), 3.85 (d, J=5.2 Hz, 2H), 3.15-3.13 (m, 4H), 2.60 (s, 3H) Note: 4H merged under solvent peak, Chemical Formula: C₂₄H₂₆Cl₂N₆O₃, Exact Mass: 516.14, Molecular Weight: 517.41. LCMS (EST) m/z=517.0/519.0 (M+H), chlorine isotopes, t_{R}: 1.23 min, 99.5% (Method-A). HPLC: 99.4%; t_{R}: 3.187 min (Method-A).

### Example 14. Synthesis of 4-methoxy-2-((3-methyl-4-(1-methylpiperidin-4-yl)phenyl)amino)-N-(2-(trifluoromethyl)phenyl)pyrimidine-5-carboxamide (Compound 114)

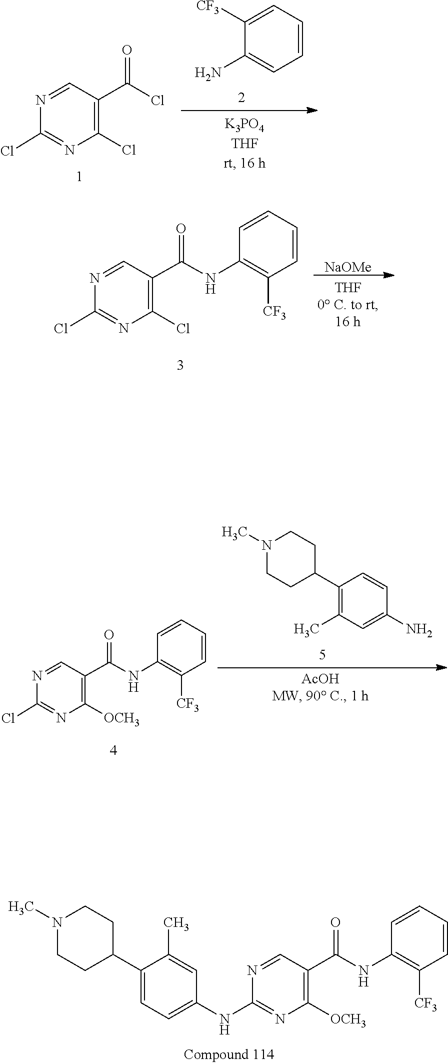

### 2,4-dichloro-N-(2-(trifluoromethyl)phenyl)pyrimidine-5-carboxamide

To a stirred solution of 2,4-dichloropyrimidine-5-carbonyl chloride (500 mg, 2.37 mmol) in THE (5 mL) was added 2-(trifluoromethyl)aniline (381 mg, 2.37 mmol) followed by potassium phosphate (1255 mg, 5.91 mmol) at room temperature under nitrogen atmosphere, and allowed to stir for 16 h. After completion of the reaction (TLC and UPLC), the mixture was diluted with water and extracted with EtOAc (60 mL×2). The combined organic layer was dried over Na₂SO₄, filtered and concentrated under reduced pressure. The resulting crude material was purified by flash chromatography (SiO₂/60-120 mesh; ~8% EtOAc/hexanes) to afford 2,4-dichloro-N-(2-(trifluoromethyl)phenyl)pyrimidine-5-carboxamide (360 mg, 41% yield) as an off-white solid. Chemical Formula: C₁₂H₆Cl₂F₃N₃O, Exact Mass: 334.98, Molecular Weight: 336.095. LCMS (ESI) m/z=333.9 (M−H), t_{R}: 1.835 min, 90.8% (Method-B).

### 2-chloro-4-methoxy-N-(2-(trifluoromethyl)phenyl)pyrimidine-5-carboxamide

To a stirred solution of 2,4-dichloro-N-(2-(trifluoromethyl)phenyl) pyrimidine-5-carboxamide (350 mg, 1.04 mmol) in THF (10 mL) at 0° C. was added sodium methanolate (56.3 mg, 1.04 mmol), and allowed to stir at rt for 16 h. After completion of the reaction (TLC and UPLC), the mixture was quenched with water and extracted with EtOAc (60 mL×2). The combined organic layer was dried over Na₂SO₄, filtered and concentrated under reduced pressure. The resulting crude material was purified by flash chromatography (SiO₂/100-200 mesh; ~8% EtOAc/hexanes) to afford 2-chloro-4-methoxy-N-(2-(trifluoromethyl)phenyl)pyrimidine-5-carboxamide (150 mg, 38% yield) as an off-white solid. ¹H-NMR (400 MHz, DMSO-d₆): δ 9.96 (s, 1H), 8.88 (s, 1H), 7.95 (d, J=8.0 Hz, 1H), 7.82-7.74 (m, 2H), 7.50 (t, J=8.0 Hz, 1H), 4.12 (s, 3H).

### 4-methoxy-2-((3-methyl-4-(1-methylpiperidin-4-yl)phenyl)amino)-N-(2-(trifluoromethyl)phenyl)pyrimidine-5-carboxamide (Compound 114)

To a stirred solution of 2-chloro-4-methoxy-N-(2-(trifluoromethyl)phenyl)pyrimidine-5-carboxamide (250 mg, 0.75 mmol) in acetic acid (4 mL) was added 3-methyl-4-(1-methylpiperidin-4-yl)aniline (154 mg, 0.75 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~10% MeOH/CH₂Cl₂) to afford 4-methoxy-2-((3-methyl-4-(1-methylpiperidin-4-yl)phenyl)amino)-N-(2(trifluoromethyl)phenyl)pyrimidine-5-carboxamide (Compound 114, 14 mg, 4% yield) as a pale-yellow solid. ¹H-NMR (400 MHz, DMSO-d₆): δ 10.18 (s, 1H), 9.38 (s, 1H), 8.87 (s, 1H), 8.28 (d, J=8.4 Hz, 1H), 7.73 (t, J=8.4 Hz, 2H), 7.62-7.60 (m, 2H), 7.39 (t, J=7.6 Hz, 1H), 7.13 (s, 1H), 4.15 (s, 3H), 3.52-3.51 (m, 2H), 3.12-2.78 (m, 6H), 2.33 (s, 3H), 1.88-1.83 (m, 4H). Chemical Formula: C₂₆H₂₈F₃N₅O₂, Exact Mass:499.22, Molecular Weight: 499.54. LCMS (ESI) m/z=500.2 (M+H), t_{R}: 1.694 min, 98% (Method-A). HPLC: 99%; t_{R}: 4.383 min (Method-A).

### Example 15. Synthesis of N-(2,6-dichloro-4-fluorophenyl)-4-methoxy-2-((3-methyl-4-(1-methylpiperidin-4-yl)phenyl)amino)pyrimidine-5-carboxamide (Compound 121)

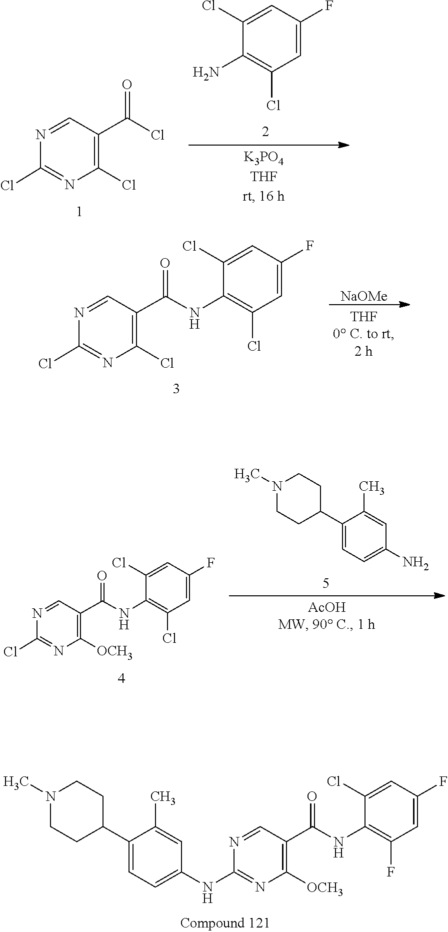

### 2,4-dichloro-N-(2,6-dichloro-4-fluorophenyl)pyrimidine-5-carboxamide

To a stirred solution of 2,4-dichloropyrimidine-5-carbonyl chloride (500 mg, 2.37 mmol) in THF (5 mL) was added 2,6-dichloro-4-fluoroaniline (426 mg, 2.37 mmol) followed by potassium phosphate (502 mg, 2.37 mmol) at room temperature under nitrogen atmosphere, and allowed to stir for 16 h. After completion of the reaction (TLC and UPLC), the mixture was diluted with water and extracted with EtOAc (60 mL×2). The combined organic layer was dried over Na₂SO₄, filtered and concentrated under reduced pressure. The resulting crude material was purified by flash chromatography (SiO₂/60-120 mesh; ~8% EtOAc/hexanes) to afford 2,4-dichloro-N-(2,6-dichloro-4-fluorophenyl)pyrimidine-5-carboxamide (350 mg, 41% yield) as an off-white solid. ¹H-NMR (400 MHz, DMSO-d₆): δ 10.81 (s, 1H), 9.02 (s, 1H), 7.72 (d, J=8.0 Hz, 2H). Chemical Formula: C₁₁H₄Cl₄FN₃O, Exact Mass: 352.91, Molecular Weight: 354.97. LCMS (ESI) m/z=351.9 (M−H), t_{R}: 1.868 min, 97.1% (Method-A).

### 2-chloro-N-(2,6-dichloro-4-fluorophenyl)-4-methoxypyrimidine-5-carboxamide

To a stirred solution of 2,4-dichloro-N-(2,6-dichloro-4-fluorophenyl)pyrimidine-5-carboxamide (340 mg, 0.96 mmol) in THF (10 mL) at 0° C. was added sodium methanolate (129 mg, 2.39 mmol), and allowed to stir at rt for 2 h. After completion of the reaction (TLC and UPLC), the mixture was quenched with water and extracted with EtOAc (60 mL×2). The combined organic layer was dried over Na₂SO₄, filtered and concentrated under reduced pressure. The resulting crude material was purified by flash chromatography (SiO₂/100-200 mesh; ~8% EtOAc/hexanes) to afford 2-chloro-N-(2,6-dichloro-4-fluorophenyl)-4-methoxypyrimidine-5-carboxamide (110 mg, 29% yield) as an off-white solid. ¹H-NMR (400 MHz, DMSO-d₆): δ 10.14 (s, 1H), 8.82 (s, 1H), 7.70 (d, J=8.4 Hz, 2H), 4.10 (s, 3H).

### N-(2,6-dichloro-4-fluorophenyl)-4-methoxy-2-((3-methyl-4-(1-methylpiperidin-4-yl)phenyl)amino)pyrimidine-5-carboxamide (Compound 121)

To a stirred solution of 2-chloro-N-(2,6-dichloro-4-fluorophenyl)-4-methoxypyrimidine-5-carboxamide (110 mg, 0.31 mmol) in acetic acid (3 mL) was added 3-methyl-4-(1-methylpiperidin-4-yl)aniline (64 mg, 0.31 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~10% MeOH/CH₂Cl₂) to afford N-(2,6-dichloro-4-fluorophenyl)-4-methoxy-2-((3-methyl-4-(1-methylpiperidin-4-yl)phenyl)amino)pyrimidine-5-carboxamide (Compound 121, 30 mg, 18% yield) as an off-white solid. ¹H-NMR (400 MHz, DMSO-d₆): δ 10.08 (s, 1H), 9.53 (s, 1H), 8.75 (s, 1H), 7.66 (d, J=8.4 Hz, 2H), 7.61-7.59 (m, 2H), 7.11 (d, J=8.4 Hz, 1H), 4.12 (s, 3H), 3.51-3.48 (m, 2H), 3.11 (s, 2H), 2.95-2.92 (m, 1H), 2.68 (s, 3H), 2.33 (s, 3H), 1.89-1.81 (m, 4H). Chemical Formula: C₂₅H₂₆Cl₂FN₅O₂, Exact Mass: 517.14, Molecular Weight: 518.41. LCMS (ESI) m/z=518.0/520.0 (M+H), chlorine isotopes, t_{R}: 1.581 min, 97.7% (Method-A). HPLC: 98%; t_{R}: 4.008 min (Method-A)

### Example 16. Synthesis of N-(2-chloro-4,6-difluorophenyl)-4-methoxy-2-((3-methyl-4-(1-methylpiperidin-4-yl)phenyl)amino)pyrimidine-5-carboxamide (Compound 127)

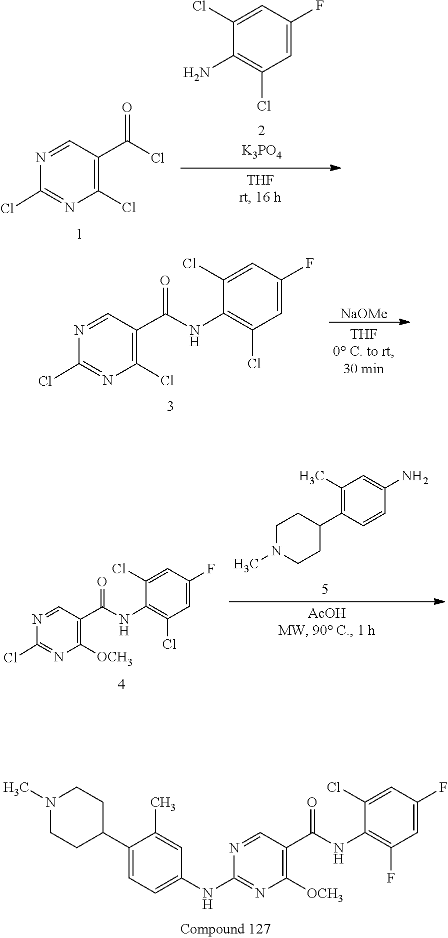

### 2,4-dichloro-N-(2-chloro-4,6-difluorophenyl)pyrimidine-5-carboxamide

To a stirred solution of 2,4-dichloropyrimidine-5-carbonyl chloride (1 g, 4.73 mmol) in THF (15 mL) was added 2-chloro-4,6-difluoroaniline (0.77 g, 4.73 mmol) followed by potassium phosphate (2.51 g, 11.82 mmol) at room temperature under nitrogen atmosphere, and allowed to stir for 16 h. After completion of the reaction (TLC and UPLC), the mixture was diluted with water and extracted with EtOAc (60 mL×2). The combined organic layer was dried over Na₂SO₄, filtered and concentrated under reduced pressure. The resulting crude material was purified by flash chromatography (SiO₂/100-200 mesh; ~8% EtOAc/hexanes) to afford 2,4-dichloro-N-(2-chloro-4,6-difluorophenyl)pyrimidine-5-carboxamide (580 mg, 36% yield) as an off-white solid. ¹H-NMR (400 MHz, DMSO-d₆): δ 10.69 (s, 1H), 9.05 (s, 1H), 7.59-7.52 (m, 2H). Chemical Formula: C₁₁H₄Cl₃F₂N₃O, Exact Mass: 336.94, Molecular Weight: 338.520. LCMS (ESI) m/z=337.8 (M+H), t_{R}: 1.74 min, 98.2% (Method-A).

### 2-chloro-N-(2-chloro-4,6-difluorophenyl)-4-methoxypyrimidine-5-carboxamide

To a stirred solution of 2,4-dichloro-N-(2-chloro-4,6-difluorophenyl)pyrimidine-5-carboxamide (250 mg, 0.73 mmol) in THF (10 mL) at 0° C. was added sodium methanolate (140 mg, 2.58 mmol), and allowed to stir at rt for 30 min. After completion of the reaction (TLC and UPLC), the mixture was quenched with water and extracted with EtOAc (60 mL×2). The combined organic layer was dried over Na₂SO₄, filtered and concentrated under reduced pressure. The resulting crude material was purified by flash chromatography (SiO₂/100-200 mesh; ~8% EtOAc/hexanes) to afford 2-chloro-N-(2-chloro-4,6-difluorophenyl)-4-methoxypyrimidine-5-carboxamide (100 mg, 36% yield) as an off-white solid. ¹H-NMR (400 MHz, DMSO-d₆): δ 9.99 (s, 1H), 8.83 (s, 1H), 7.56-7.48 (m, 2H), 4.10 (s, 3H).

### N-(2-chloro-4,6-difluorophenyl)-4-methoxy-2-((3-methyl-4-(1-methylpiperidin-4-yl)phenyl)amino)pyrimidine-5-carboxamide (Compound 127)

To a stirred solution of 2-chloro-N-(2-chloro-4,6-difluorophenyl)-4-methoxypyrimidine-5-carboxamide (100 mg, 0.29 mmol) in acetic acid (3 mL) was added 3-methyl-4-(1-methylpiperidin-4-yl)aniline (62 mg, 0.29 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/230-400 mesh; ~8% MeOH/CH₂Cl₂) to afford N-(2-chloro-4,6-difluorophenyl)-4-methoxy-2-((3-methyl-4-(1-methylpiperidin-4-yl)phenyl)amino)pyrimidine-5-carboxamide (Compound 127, 14 mg, 9% yield) as an off-white solid. ¹H-NMR (400 MHz, DMSO-d₆): δ 10.07 (s, 1H), 9.39 (s, 1H), 8.75 (s, 1H), 7.60-7.59 (m, 2H), 7.51-7.45 (m, 2H), 7.14 (d, J=8.4 Hz, 1H), 4.11 (s, 3H), 2.85 (s, 3H), 2.69-2.67 (m, 5H), 2.34-2.32 (m, 3H), 1.91-1.81 (m, 4H). Chemical Formula: C₂₅H₂₆C₁F₂N₅O₂, Exact Mass: 501.17, Molecular Weight: 501.96. LCMS (ESI) m/z=502.2/504.2 (M+H), chlorine isotopes, t_{R}: 1.493 min, 96.8% (Method-A). HPLC: 96.5%; t_{R}: 3.922 min (Method-A).

### Example 17. Synthesis of N-(2-chloro-3,6-difluorophenyl)-4-methoxy-2-((3-methyl-4-(1-methylpiperidin-4-yl)phenyl)amino)pyrimidine-5-carboxamide (Compound 128)

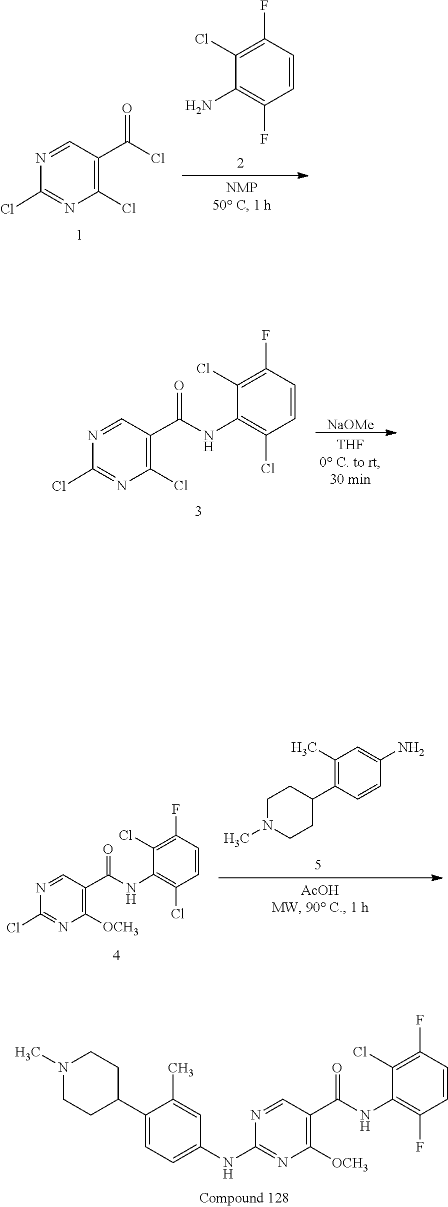

### 2,4-dichloro-N-(2-chloro-3,6-difluorophenyl)pyrimidine-5-carboxamide

To a stirred solution of 2,4-dichloropyrimidine-5-carbonyl chloride (1 g, 4.73 mmol) in NMP (10 mL) was added 2-chloro-3,6-difluoroaniline (0.77 g, 4.73 mmol) under nitrogen atmosphere, and allowed to stir at 50° C. for 1 h. After completion of the reaction (TLC and UPLC), the mixture was diluted with water and extracted with EtOAc (60 mL×2). The combined organic layer was dried over Na₂SO₄, filtered and concentrated under reduced pressure. The resulting crude material was purified by flash chromatography (SiO₂/100-200 mesh; ~8% EtOAc/hexanes) to afford 2,4-dichloro-N-(2-chloro-3,6-difluorophenyl)pyrimidine-5-carboxamide (870 mg, 54% yield) as an off-white solid. ¹H-NMR (400 MHz, DMSO-d₆): δ 10.89 (s, 1H), 9.07 (s, 1H), 7.59-7.46 (m, 2H).

### 2-chloro-N-(2-chloro-3,6-difluorophenyl)-4-methoxypyrimidine-5-carboxamide

To a stirred solution of 2,4-dichloro-N-(2-chloro-3,6-difluorophenyl)pyrimidine-5-carboxamide (350 mg, 1.03 mmol) in THF (20 ml) at 0° C. was added sodium methanolate (195 mg, 3.62 mmol), and allowed to stir at rt for 30 min. After completion of the reaction (TLC and UPLC), the mixture was quenched with water and extracted with EtOAc (60 mL×2). The combined organic layer was dried over Na₂SO₄, filtered and concentrated under reduced pressure. The resulting crude material was purified by flash chromatography (SiO₂/100-200 mesh; ~8% EtOAc/hexanes) to afford 2-chloro-N-(2-chloro-3,6-difluorophenyl)-4-methoxypyrimidine-5-carboxamide (130 mg, 34% yield) as an off-white solid. ¹H-NMR (400 MHz, DMSO-d₆): δ 10.17 (s, 1H), 8.83 (s, 1H), 7.56-7.43 (m, 2H), 4.10 (s, 3H). Chemical Formula: C₁₂H₇Cl₂F₂N₃O₂, Exact Mass: 332.99, Molecular Weight: 334.104. LCMS (ESI) m/z=334.0/336.0 (M+H), chlorine isotopes, t_{R}: 1.985 min, 92% (Method-A).

### N-(2-chloro-3,6-difluorophenyl)-4-methoxy-2-((3-methyl-4-(1-methylpiperidin-4-yl)phenyl)amino)pyrimidine-5-carboxamide (Compound 128)

To a stirred solution of 2-chloro-N-(2-chloro-3,6-difluorophenyl)-4-methoxypyrimidine-5-carboxamide (130 mg, 0.38 mmol) in acetic acid (3 mL) was added 3-methyl-4-(1-methylpiperidin-4-yl)aniline (79 mg, 0.38 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/230-400 mesh; ~8% MeOH/CH₂Cl₂) to afford N-(2-chloro-3,6-difluorophenyl)-4-methoxy-2-((3-methyl-4-(1-methylpiperidin-4-yl)phenyl)amino)pyrimidine-5-carboxamide (Compound 128, 50 mg, 25% yield) as an off-white solid. ¹H-NMR (400 MHz, DMSO-d₆): δ 10.10 (s, 1H), 9.54 (s, 1H), 8.75 (s, 1H), 7.62-7.60 (m, 2H), 7.53-7.39 (m, 2H), 7.13 (d, J=7.6 Hz, 1H), 4.12 (s, 3H), 2.85 (br s, 3H), 2.69-2.67 (m, 5H), 2.34-2.32 (m, 3H), 1.91-1.86 (m, 4H). Chemical Formula: C₂₅H₂₆ClF₂N₅O₂, Exact Mass: 501.17, Molecular Weight: 501.96. LCMS (ESI) m/z=502.2/504.2 (M+H), chlorine isotopes, t_{R}: 1.536 min, 98.9% (Method-A). HPLC: 99.8%; t_{R}: 3.917 min (Method-A).

### Example 18. Synthesis of N-(2,6-dichlorophenyl)-4-methoxy-2-((4-(4-methylpiperazin-1-yl)phenyl) amino)pyrimidine-5-carboxamide (Compound 111)

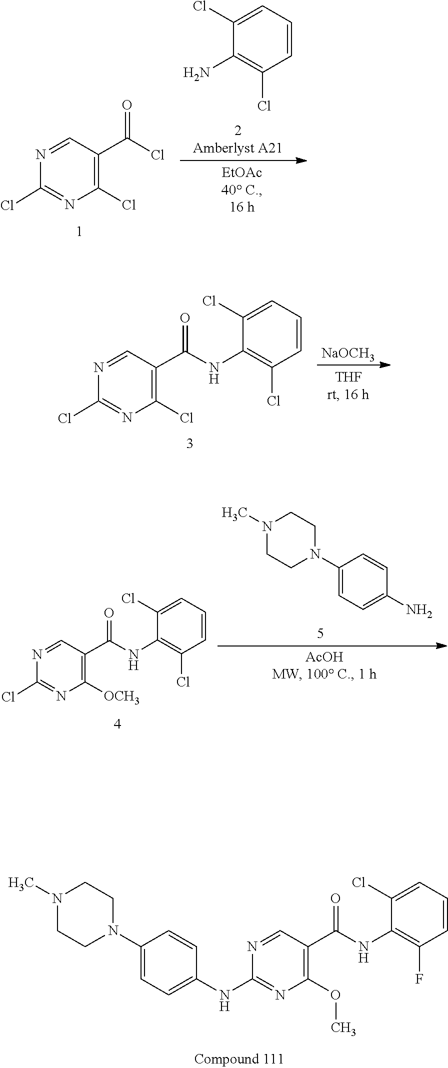

### 2,4-dichloro-N-(2,6-dichlorophenyl)pyrimidine-5-carboxamide

To a stirred solution of 2,4-dichloropyrimidine-5-carbonyl chloride (2 g, 9.46 mmol) in ethyl acetate (160 mL), was added 2,6-dichloroaniline (1.533 g, 9.46 mmol) followed by the addition of Amberlyst A 21 Free base (400 mg, 9.46 mmol) at room temperature under nitrogen atmosphere. Then, the reaction mixture was allowed to stir at 40° C. for 16 h. After completion of the reaction (TLC and UPLC), the reaction mixture was diluted with water and extracted with EtOAc (50 mL×2). The combined organic layer was dried over Na₂SO₄, filtered and concentrated under reduced pressure. The resulting crude material was washed with MTBE to get 2,4-dichloro-N-(2,6-dichlorophenyl)pyrimidine-5-carboxamide (0.9 g, 28% yield) as an off-white solid. ¹H-NMR (DMSO-d₆, 400 MHz): δ 10.85 (s, 1H), 9.02 (s, 1H), 7.63 (d, J=8.0 Hz, 2H), 7.45 (t, J=8.0 Hz, 1H). Chemical Formula: Cn HSCl₄N₃O, Exact Mass: 334.92, Molecular Weight: 336.98. LCMS (ESI) m/z=336.0/338.0/340.0 (M+H) chlorine isotopes, t_{R}. 1.886 min, 99.8% (Method A).

### 2-chloro-N-(2,6-dichlorophenyl)-4-methoxypyrimidine-5-carboxamide

To a stirred solution of 2,4-dichloro-N-(2,6-dichlorophenyl)pyrimidine-5-carboxamide (250 mg, 0.742 mmol) in tetrahydrofuran (10 mL), was added sodium methoxide (42.1 mg, 0.779 mmol) at 0° C. The resulting reaction mixture was stirred at room temperature for 16 h. After completion of the reaction (TLC and UPLC), the reaction mixture was diluted with water and extracted with CH₂Cl₂ (50 mL×2). The combined organic layer was dried over Na₂SO₄, filtered and concentrated under reduced pressure. The resulting crude material was purified by flash chromatography (SiO₂/100-200 mesh; 0-10% CH₃OH/CH₂Cl₂) to afford 2-chloro-N-(2,6-dichlorophenyl)-4-methoxypyrimidine-5-carboxamide (500 mg, 84% yield) as an off-white solid. ¹H-NMR (DMSO-d₆, 400 MHz): δ 10.18 (s, 1H), 8.82 (s, 1H), 7.60 (d, J=8.0 Hz, 2H), 7.42 (t, J=8.0 Hz, 1H), 4.10 (s, 3H). Chemical Formula: C₁₂H₈Cl₃N₃O₂, Exact Mass: 330.97, Molecular Weight: 332.57. LCMS (ESI) m/z=332.2/334.0/335.0 (M+H) chlorine isotopes, t_{R}. 1.901 min, 81.4% (Method A).

### N-(2,6-dichlorophenyl)-4-methoxy-2-((4-(4-methylpiperazin-1-yl)phenyl)amino)pyrimidine-5-carboxamide [SYE2102581-43; (DS19-TM32)]

To a stirred solution of 2-chloro-N-(2,6-dichlorophenyl)-4-methoxypyrimidine-5-carboxamide (150 mg, 0.451 mmol) in acetic acid (3 mL) was added 4-(4-methylpiperazin-1-yl)aniline (104 mg, 0.541 mmol) at room temperature under nitrogen atmosphere. The resultant reaction mixture was subjected to microwave irradiation at 100° C. for 1 h. After completion of the reaction (TLC and UPLC), the reaction mixture was diluted with water and extracted with ethyl acetate (50 mL×2). The combined organic layer was dried over Na₂SO₄, filtered and concentrated under reduced pressure. The resulting crude material was purified by flash chromatography (SiO₂/230-400 mesh; 0-15% CH₃₀H/CH₂C₁₂) to afford N-(2,6-dichlorophenyl)-4-methoxy-2-((4-(4-methylpiperazin-1-yl)phenyl)amino)pyrimidine-5-carboxamide (Compound 111, 40 mg, 18% yield) as an off-white solid. ¹H NMR (400 MHz, DMSO-d₆): δ 9.96 (br s, 1H), 9.52 (s, 1H), 8.72 (s, 1H), 7.63-7.57 (m, 4H), 7.39 (t, J=8.00 Hz, 1H), 6.95 (d, J=9.20 Hz, 2H), 4.09 (s, 3H), 3.21-3.10 (m, 4H), 2.68-2.65 (m, 4H), 2.37 (s, 3H). Chemical Formula: C₂₃H₂₄Cl₂N₆O₂, Exact Mass: 486.13, Molecular Weight: 487.39. LCMS (ESI) m/z=487.0/489.0/491.0 (M+H) chlorine isotopes, t_{R}. 1.378 min, 99.9% (Method-C), HPLC: 99.8%, t_{R}: 3.614 min, (Method-B).

### Example 19. Synthesis of N-(2,6-dichlorophenyl)-4-methoxy-2-((2-methyl-1,2,3,4-tetrahydro isoquinolin-7-yl)amino)pyrimidine-5-carboxamide (Compound 116)

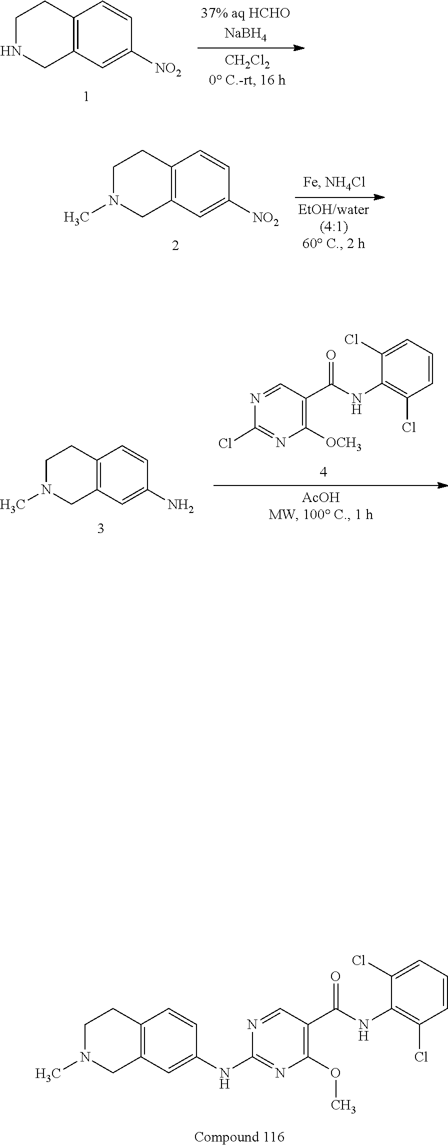 [SMILES: O=[N+]([O-])c1ccc2c(c1)CNCC2 ; CN1CCc2ccc([N+](=O)[O-])cc2C1 ; COc1nc(Cl)ncc1C(=O)Nc1c(Cl)cccc1Cl ; CN1CCc2ccc(N)cc2C1 ; COc1nc(Nc2ccc3c(c2)CN(C)CC3)ncc1C(=O)Nc1c(Cl)cccc1Cl]

### 2-methyl-7-nitro-1,2,3,4-tetrahydroisoquinoline

To a stirred solution of 7-nitro-1,2,3,4-tetrahydroisoquinoline (1 g, 5.61 mmol) in CH₂Cl₂ (60 mL), were added formaldehyde (37% in water), (0.43 mL, 5.61 mmol), acetic acid (2 drops) at 0° C. The resulting reaction mixture was stirred at room temperature for 30 minutes. To this reaction mixture sodium borohydride (0.213 g, 5.61 mmol) was added at room temperature. The resulting reaction mixture was stirred at room temperature for 16 h. After completion of the reaction (TLC), the reaction mixture was diluted with water and extracted with EtOAc (50 mL×2). The combined organic layer was dried over Na₂SO₄, filtered and concentrated under reduced pressure. The resulting crude material was purified by flash chromatography (SiO₂/100-200 mesh; CH₃OH/CH₂Cl₂) to afford 2-methyl-7-nitro-1,2,3,4-tetrahydroisoquinoline (700 mg, 65% yield) as a yellow solid. δ 1H-NMR (400 MHz, DMSO-d₆): δ 8.00-7.98 (m, 2H), 7.40 (d, J=8.80 Hz, 1H), 3.60 (br s, 2H), 2.94 (t, J=6.00 Hz, 2H), 2.63 (t, J=6.00 Hz, 2H), 2.36 (s, 3H). Chemical Formula: CioH₁₂N₂O₂, Exact Mass: 192.09, Molecular Weight: 192.22. Note: LCMS product did not ionize.

### 2-methyl-1,2,3,4-tetrahydroisoquinolin-7-amine

To a stirred solution of 2-methyl-6-nitro-1,2,3,4-tetrahydroisoquinoline (350 mg, 1.821 mmol) in ethanol (2 mL), water (0.5 mL) was added iron powder (509 mg, 9.10 mmol) followed by the addition of ammonium chloride (972 mg, 18.21 mmol) at room temperature. The resulting reaction mixture was stirred at 60° C. for 2 h. After completion of the reaction (TLC and UPLC), the reaction mixture was diluted with saturated sodium bicarbonate solution and extracted with CH₂Cl₂ (50 mL×2). The combined organic layer was dried over Na₂SO₄, filtered and concentrated under reduced pressure to get 22-methyl-1,2,3,4-tetrahydroisoquinolin-7-amine (250 mg, 85% yield)) as brown liquid that was taken into the next step without further purification. ¹H-NMR (400 MHz, DMSO-d₆): δ 6.73 (d, J=8.40 Hz, 1H), 6.36 (dd, J=2.00, 8.20 Hz, 1H), 6.22 (d, J=2.00 Hz, 1H), 4.76 (br s, 2H), 3.33 (s, 2H), 2.62-2.61 (m, 2H), 2.29 (s, 3H). Note: 2 protons were merged with solvent peak. Chemical Formula: C₁₀H₁₄N₂, Exact Mass: 162.12, Molecular Weight: 162.24. LCMS (ESI) m/z=163.1 (M+H) chlorine isotopes, t_{R}. 0.971 min, 98.8% (Method A).

### N-(2,6-dichlorophenyl)-4-methoxy-2-((2-methyl-1,2,3,4-tetrahydro isoquinolin-7-yl)amino)pyrimidine-5-carboxamide (Compound 116)

To a stirred solution of 2-chloro-N-(2,6-dichlorophenyl)-4-methoxypyrimidine-5-carboxamide (150 mg, 0.451 mmol) in acetic acid (3.5 mL), was added a 2-methyl-1,2,3,4-tetrahydroisoquinolin-7-amine (88 mg, 0.541 mmol) at room temperature under nitrogen atmosphere. The resultant reaction mixture was subjected to microwave irradiation at 100° C. for 1 h. After completion of the reaction (TLC and UPLC), the reaction mixture was diluted with water and extracted with ethyl acetate (50 mL×2). The combined organic layer was washed with saturated aqueous NaHCO₃, dried over Na₂SO₄, filtered and concentrated under reduced pressure. The resulting crude material was purified by flash chromatography (SiO₂/230-400 mesh; 0-15% CH₃OH/CH₂Cl₂) to afford N-(2,6-dichlorophenyl)-4-methoxy-2-((2-methyl-1,2,3,4-tetrahydroisoquinolin-7-yl)amino)pyrimidine-5-carboxamide (Compound 116, 30 mg, 15% yield) as an off-white solid. ¹H-NMR (400 MHz, DMSO-d₆): δ 10.09 (br s, 1H), 9.56 (s, 1H), 8.75 (s, 1H), 7.59-7.57 (m, 3H), 7.55-7.48 (m, 1H), 7.41-7.39 (m, 1H), 7.08 (d, J=8.00 Hz, 1H), 4.10 (s, 3H), 3.55 (s, 2H), 2.80-2.79 (m, 2H), 2.68-2.66 (m, 2H), 2.39 (s, 3H). Chemical Formula: C₂₂H₂₁Cl₂N₅O₂, Exact Mass: 457.11, Molecular Weight: 458.34. LCMS (ESI) m/z=458.2/460.2 (M+H) chlorine isotopes, t_{R}=1.402 min, 97.1% (Method-A), HPLC purity: 99.7%; t_{R}=3.544 min.

### Example 20. Synthesis of N-(2,6-dichlorophenyl)-4-(2-hydroxypropoxy)-2-((4-(4-methylpiperazin-1-yl)phenyl)amino)pyrimidine-5-carboxamide (Compound 119) and N-(2,6-dichlorophenyl)-4-(3-(dimethylamino)propoxy)-2-((4-(4-methylpiperazin-1-yl)phenyl)amino)pyrimidine-5-carboxamide (Compound 124)

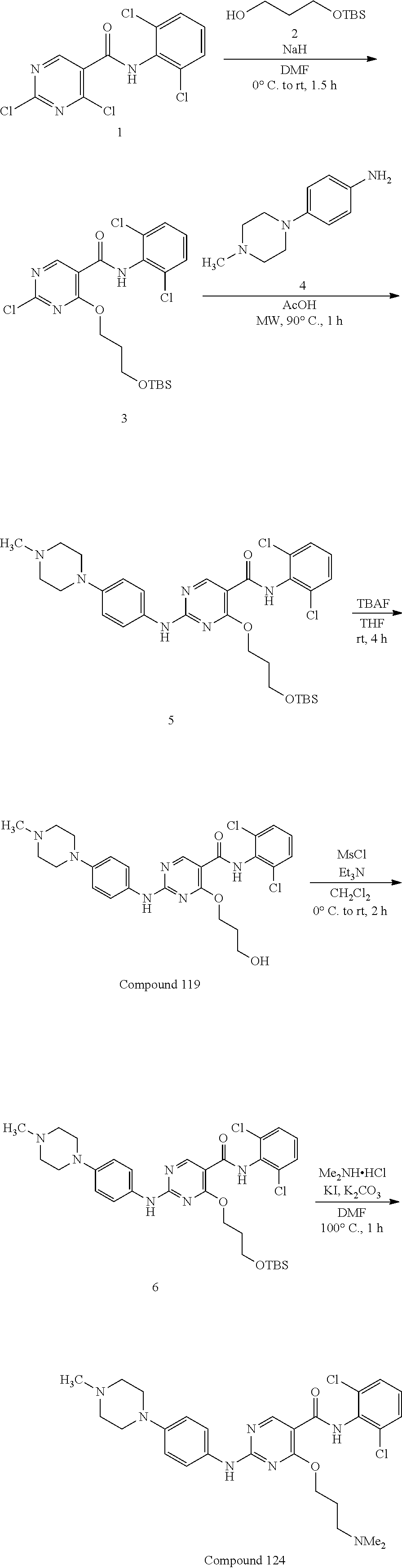

### 4-(2-((tert-butyldimethylsilyl)oxy)propoxy)-2-chloro-N-(2,6-dichlorophenyl)pyrimidine-5-carboxamide

To a stirred solution of 2-((tert-butyldimethylsilyl)oxy)propan-1-ol (621 mg, 3.26 mmol) in DMF (15 mL) at 0° C. was added sodium hydride (862 mg, 5.34 mmol) and the reaction mixture was stirred for 30 min. To this solution 2,4-dichloro-N-(2,6-dichlorophenyl)pyrimidine-5-carboxamide (1 g, 2.97 mmol) was added at 0° C. The resulting reaction mixture was stirred at rt for 1 h. After completion of the reaction (TLC and UPLC), the mixture was quenched with saturated aq. NH₄Cl solution and extracted with EtOAc (100 mL×2). The combined organic layer was dried over Na₂SO₄, filtered and concentrated under reduced pressure. The resulting crude material was purified by flash chromatography (SiO₂/230-400 mesh; ~12% EtOAc/hexanes) to afford 4-(2-((tert-butyldimethylsilyl)oxy)propoxy)-2-chloro-N-(2,6-dichlorophenyl)pyrimidine-5-carboxamide (800 mg, 55% yield) as an off-white solid. ¹H-NMR (400 MHz, DMSO-d₆): δ 10.13 (s, 1H), 8.77 (s, 1H), 7.60 (t, J=6.4 Hz, 2H), 7.42 (t, J=8.0 Hz, 1H), 4.57 (t, J=6.4 Hz, 2H), 3.76 (t, J=6.0 Hz, 2H), 2.02 (t, J=6.4 Hz, 2H), 0.84 (s, 9H), 0.03 (s, 6H).

### 4-(2-((tert-butyldimethylsilyl)oxy)propoxy)-N-(2,6-dichlorophenyl)-2-((4-(4-methylpiperazin-1-yl)phenyl)amino)pyrimidine-5-carboxamide

To a stirred solution of 4-(2-((tert-butyldimethylsilyl)oxy)propoxy)-2-chloro-N-(2,6-dichlorophenyl)pyrimidine-5-carboxamide (550 mg, 1.12 mmol) in acetic acid (5 mL) was added 4-(4-methylpiperazin-1-yl)aniline (236 mg, 1.23 mmol), and the resulting reaction mixture was subjected to microwave irradiation at 90° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (30 mL×3), and the combined organic layer was dried over Na₂SO₄, filtered and concentrated under reduced pressure. The resulting crude material was purified by flash chromatography (SiO₂/230-400 mesh; ~10% MeOH/CH₂Cl₂) to afford 4-(2-((tert-butyldimethylsilyl)oxy)propoxy)-N-(2,6-dichlorophenyl)-2-((4-(4-methylpiperazin-1-yl)phenyl)amino)pyrimidine-5-carboxamide (450 mg, 62% yield) as an off-white solid. Chemical Formula: C₃₁H₄₂C₁₂N₆O₃Si, Exact Mass: 644.25, Molecular Weight: 645.701. LCMS (ESI) m/z=645.2/647.2 (M+H), chlorine isotopes, t_{R}: 1.952 min, 60.2% (Method-A).

### N-(2,6-dichlorophenyl)-4-(2-hydroxypropoxy)-2-((4-(4-methylpiperazin-1-yl)phenyl)amino)pyrimidine-5-carboxamide (Compound 119)

To a stirred solution of 4-(2-((tert-butyldimethylsilyl)oxy)propoxy)-N-(2,6-dichlorophenyl)-2-((4-(4-methylpiperazin-1-yl)phenyl)amino)pyrimidine-5-carboxamide (300 mg, 0.47 mmol) in THE (5 mL) at 0° C. was added TBAF (1M in THF) (0.67 mL, 0.67 mmol), and allowed to stir at rt for 4 h. After completion of the reaction (TLC and UPLC), the volatiles were removed under reduced pressure and the crude residue was diluted with saturated aq. NH₄Cl solution. The aqueous layer was extracted with CH₂Cl₂ (30 mL×3), and the combined organic layer was dried over Na₂SO₄, filtered and concentrated under reduced pressure. The resulting crude material was purified by flash chromatography (SiO₂/230-400 mesh; ~12% MeOH/CH₂Cl₂) to afford N-(2,6-dichlorophenyl)-4-(2-hydroxypropoxy)-2-((4-(4-methylpiperazin-1-yl)phenyl)amino)pyrimidine-5-carboxamide (Compound 119, 190 mg, 90% yield) as an off-white solid. ¹H-NMR (400 MHz, Methanol-d₄): δ 8.84 (s, 1H), 7.63 (d, J=9.2 Hz, 2H), 7.52 (d, J=8.0 Hz, 2H), 7.35 (t, J=8.8 Hz, 1H), 7.05 (d, J=9.2 Hz, 2H), 4.75 (t, J=6.0 Hz, 2H), 3.79 (t, J=6.0 Hz, 2H), 3.37-3.35 (m, 3H), 3.21-3.19 (m, 4H), 2.78 (s, 3H), 2.11 (t, J=6.00 Hz, 2H) Note: 4H merged under solvent peak. Chemical Formula: C₂₅H₂₈Cl₂N₆O₃, Exact Mass: 530.16, Molecular Weight: 531.43. LCMS (ESI) im/z=531.1/533.1 (M+H), chlorine isotopes, t_{R}: 1.374 min, 98.3% (Method-A). HPLC: 99.5%; t_{R}: 3.738 min (Method-A).

### 2-((5-((2,6-dichlorophenyl)carbamoyl)-2-((4-(4-methylpiperazin-1-yl)phenyl)amino)pyrimidin-4-yl)oxy)propyl methanesulfonate

To a stirred of N-(2,6-dichlorophenyl)-4-(2-hydroxypropoxy)-2-((4-(4-methylpiperazin-1-yl)phenyl)amino)pyrimidine-5-carboxamide (200 mg, 0.38 mmol) in CH₂Cl₂ (10 mL) at 0° C. was added triethylamine (112 mg, 1.13 mmol) followed by mesyl chloride (63 mg, 0.56 mmol), and allowed to stir at rt for 2 h. After completion of the reaction (TLC and UPLC), the volatiles were removed under reduced pressure and the crude residue was diluted with saturated aq. NH₄Cl solution. The aqueous layer was extracted with CH₂Cl₂ (30 mL×3), and the combined organic layer was dried over Na₂SO₄, filtered and concentrated under reduced pressure to afford crude 2-((5-((2,6-dichlorophenyl)carbamoyl)-2-((4-(4-methylpiperazin-1-yl)phenyl)amino)pyrimidin-4-yl)oxy)propyl methanesulfonate (200 mg, 81% yield) as an off-white solid. Chemical Formula: C₂₆H₃₀Cl₂N₆O₅S, Exact Mass: 608.14, Molecular Weight: 609.52. LCMS (ESI) m/z=607.2 (M−H), t_{R}: 1.389 min, 86.4% (Method-A).

### N-(2,6-dichlorophenyl)-4-(2-(dimethylamino)propoxy)-2-((4-(4-methylpiperazin-1-yl)phenyl)amino)pyrimidine-5-carboxamide (Compound 124)

To a stirred solution of dimethyl amine hydrochloride (66.9 mg, 0.82 mmol) in DMF (4 mL) was added potassium carbonate (232 mg, 1.68 mmol) followed by potassium iodide (84 mg, 0.51 mmol) and stirred at rt for 5 min. To this solution 2-((5-((2,6-dichlorophenyl)carbamoyl)-2-((4-(4-methylpiperazin-1-yl)phenyl)amino)pyrimidin-4-yl)oxy)propyl methanesulfonate (200 mg, 0.33 mmol) was added and stirred at 100° C. for 1 h. After completion of the reaction (TLC and UPLC), the volatiles were removed under reduced pressure and the crude residue was diluted with water. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/230-400 mesh; ~22% MeOH/CH₂Cl₂) to afford N-(2,6-dichlorophenyl)-4-(2-(dimethylamino)propoxy)-2-((4-(4-methylpiperazin-1-yl)phenyl)amino)pyrimidine-5-carboxamide (Compound 124, 50 mg, 27% yield) as an off-white solid. ¹H-NMR (400 MHz, Methanol-d₄): δ 8.82 (s, 1H), 7.58-7.56 (m, 2H), 7.55-7.53 (m, 2H), 7.39-7.35 (m, 1H), 7.04-7.01 (m, 2H), 4.68 (t, J=6.0 Hz, 2H), 3.23-3.21 (m, 4H), 2.98-2.95 (m, 2H), 2.73-2.69 (m, 4H), 2.53 (s, 6H), 2.44 (s, 3H), 2.23-2.17 (m, 2H). Note: exchangeable protons not observed. Chemical Formula: C₂₇H₃₃Cl₂N₇O₂, Exact Mass: 557.21, Molecular Weight: 558.51. LCMS (ESI) im/z=558.2/560.2 (M+H), chlorine isotopes, t_{R}: 1.109 min, 95.1% (Method-A). HPLC: 95.6%; t_{R}: 3.212 min (Method-A).

### Example 21. Synthesis of N-(6-chloro-2,3-difluorophenyl)-4-methoxy-2-((3-methyl-4-(1-methylpiperidin-4-yl)phenyl)amino)pyrimidine-5-carboxamide (Compound 125)

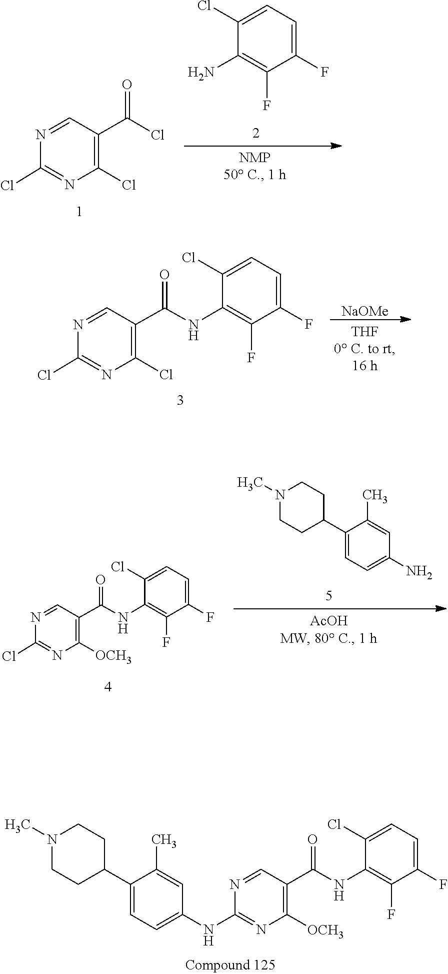

### 2,4-dichloro-N-(6-chloro-2,3-difluorophenyl)pyrimidine-5-carboxamide

To a stirred solution of 2,4-dichloropyrimidine-5-carbonyl chloride (300 mg, 1.41 mmol) in NMP (3 mL) was added 6-chloro-2,3-difluoroaniline (255 mg, 1.56 mmol) at room temperature under nitrogen atmosphere, and allowed to stir at 50° C. for 1 h. After completion of the reaction (TLC and UPLC), the mixture was diluted with water and extracted with EtOAc (50 mL×2). The combined organic layer was dried over Na₂SO₄, filtered and concentrated under reduced pressure. The resulting crude material was purified by flash chromatography (SiO₂/60-120 mesh; ~8% EtOAc/hexanes) to afford 2,4-dichloro-N-(6-chloro-2,3-difluorophenyl)pyrimidine-5-carboxamide (300 mg, 63% yield) as an off-white solid. ¹H-NMR (400 MHz, DMSO-d₆): δ 10.91 (s, 1H), 9.07 (s, 1H), 7.59-7.53 (m, 2H)
Chemical Formula: C₁₁H₄Cl₃F₂N₃O, Exact Mass: 336.94, Molecular Weight: 338.52

LCMS (ESI) m/z=337.8/339.8 (M+H), chlorine isotopes, t_{R}: 1.760 min, 91.0% (Method-A)

### 2-chloro-N-(6-chloro-2,3-difluorophenyl)-4-methoxypyrimidine-5-carboxamide

To a stirred solution of 2,4-dichloro-N-(2-chloro-6-fluorophenyl)pyrimidine-5-carboxamide (300 mg, 0.88 mmol) in THF (10 mL) at 0° C. was added sodium methanolate (57.5 mg, 1.06 mmol), and allowed to stir at rt for 16 h. After completion of the reaction (TLC and UPLC), the mixture was quenched with water and extracted with EtOAc (40 mL×2). The combined organic layer was dried over Na₂SO₄, filtered and concentrated under reduced pressure. The resulting crude material was purified by flash chromatography (SiO₂/100-200 mesh; ~12% EtOAc/hexanes) to afford 2-chloro-N-(2-chloro-6-fluorophenyl)-4-methoxypyrimidine-5-carboxamide (120 mg, 40% yield) as an off-white solid. ¹H-NMR (400 MHz, DMSO-d₆): δ 10.18 (s, 1H), 8.83 (s, 1H), 7.57-7.51 (m, 2H), 4.10 (s, 3H).

### N-(6-chloro-2,3-difluorophenyl)-4-methoxy-2-((3-methyl-4-(1-methylpiperidin-4-yl)phenyl)amino)pyrimidine-5-carboxamide (Compound 125)

To a stirred solution of 2-chloro-N-(2-chloro-6-fluorophenyl)-4-methoxypyrimidine-5-carboxamide (100 mg, 0.29 mmol) in acetic acid (3.5 mL) was added 3-methyl-4-(1-methylpiperidin-4-yl)aniline (61 mg, 0.29 mmol), and the resulting reaction mixture was subjected to microwave irradiation at 80° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/230-400 mesh; ~10% MeOH/CH₂Cl₂) to afford N-(6-chloro-2,3-difluorophenyl)-4-methoxy-2-((3-methyl-4-(1-methylpiperidin-4-yl)phenyl)amino)pyrimidine-5-carboxamide (Compound 125, 30 mg, 20% yield) as an off-white solid. ¹H-NMR (400 MHz, DMSO-d₆): δ 10.10 (s, 1H), 9.55 (s, 1H), 8.58 (s, 1H), 7.51-7.60 (m, 4H), 7.15 (d, J=8.8 Hz, 1H), 4.12 (s, 3H), 3.30-3.26 (m, 2H), 2.82-2.68 (m, 2H), 2.57-2.53 (m, 3H), 2.31-2.33 (m, 4H), 1.89-1.79 (m, 4H). Chemical Formula: C₂₅H₂₆C₁F₂N₅O₂, Exact Mass: 501.17, Molecular Weight: 501.96. LCMS (ESI) m/z=502.2/504.2 (M+H), chlorine isotopes, t_{R}: 1.517 min, 99.6% (Method-A). HPLC: 98.2%; t_{R}: 5.955 min (Method-A).

### Example 22. Synthesis of N-(2-chloro-6-(trifluoromethyl)phenyl)-4-methoxy-2-((3-methyl-4-(1-methylpiperidin-4-yl)phenyl)amino)pyrimidine-5-carboxamide (Compound 122)

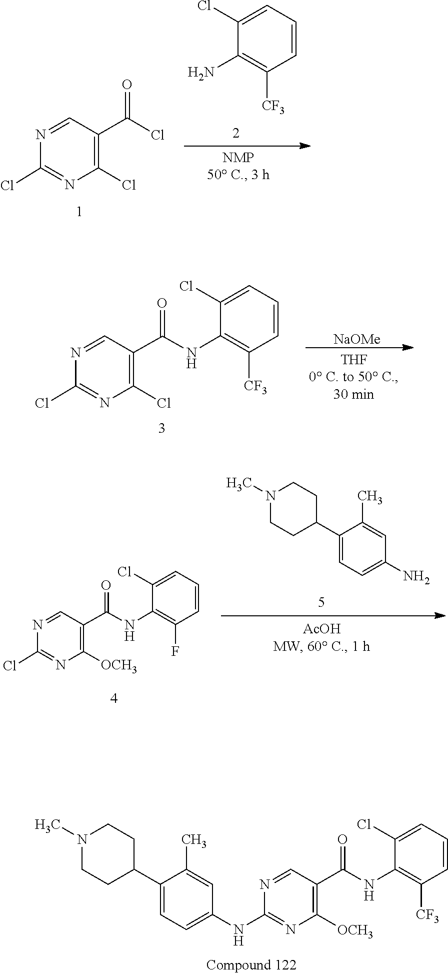

### 2,4-dichloro-N-(2-chloro-6-(trifluoromethyl)phenyl)pyrimidine-5-carboxamide

To a stirred solution of 2,4-dichloropyrimidine-5-carbonyl chloride (400 mg, 1.89 mmol) in NMP (10 mL) was added 2-chloro-6-(trifluoromethyl)aniline (370 mg, 1.89 mmol) at room temperature under nitrogen atmosphere, and allowed to stir at 50° C. for 3 h. After completion of the reaction (TLC and UPLC), the mixture was diluted with water and extracted with EtOAc (50 mL×2). The combined organic layer was dried over Na₂SO₄, filtered and concentrated under reduced pressure. The resulting crude material was purified by flash chromatography (SiO₂/230-400 mesh; ~16% EtOAc/hexanes) to afford 2,4-dichloro-N-(2-chloro-6-(trifluoromethyl)phenyl)pyrimidine-5-carboxamide (250 mg, 35% yield) as an off-white solid. ¹H-NMR (400 MHz, DMSO-d₆): δ 10.90 (s, 1H), 8.93 (s, 1H), 8.01 (dd, J=8.0, 0.4 Hz, 1H), 7.86 (dd, J=7.6, 0.8 Hz, 1H), 7.67 (t, J=8.0 Hz, 1H).

### 2-chloro-N-(2-chloro-6-(trifluoromethyl)phenyl)-4-methoxypyrimidine-5-carboxamide

To a stirred solution of 2,4-dichloro-N-(2-chloro-6-(trifluoromethyl)phenyl)pyrimidine-5-carboxamide (200 mg, 0.54 mmol) in THF (8 mL) at 0° C. was added sodium methanolate (73 mg, 1.35 mmol), and allowed to stir at 50° C. for 30 min. After completion of the reaction (TLC and UPLC), the mixture was quenched with water and extracted with EtOAc (40 mL×2). The combined organic layer was dried over Na₂SO₄, filtered and concentrated under reduced pressure. The resulting crude material was purified by flash chromatography (SiO₂/100-200 mesh; ~25% EtOAc/hexanes) to afford 2-chloro-N-(2-chloro-6-(trifluoromethyl)phenyl)-4-methoxypyrimidine-5-carboxamide (60 mg, 29% yield) as an off-white solid. ¹H-NMR (400 MHz, DMSO-d₆): δ 10.23 (s, 1H), 8.77 (s, 1H), 7.97 (dd, J=0.8, 8.0 Hz, 1H), 7.83 (dd, J=0.8, 8.0 Hz, 1H), 7.64 (t, J=8.0 Hz, 1H), 4.09 (s, 3H). Chemical Formula: C₁₃H₈Cl₂F₃N₃O₂, Exact Mass: 364.99, Molecular Weight: 366.12. LCMS (ESI) m/z=366.2/368.0 (M+H), chlorine isotopes, t_{R}: 1.956 min, 95.3% (Method-A).

### N-(2-chloro-6-(trifluoromethyl)phenyl)-4-methoxy-2-((3-methyl-4-(1-methylpiperidin-4-yl)phenyl)amino)pyrimidine-5-carboxamide (Compound 122)

To a stirred solution of 2-chloro-N-(2-chloro-6-(trifluoromethyl)phenyl)-4-methoxypyrimidine-5-carboxamide (90 mg, 0.25 mmol) in acetic acid (3 mL) was added 3-methyl-4-(1-methylpiperidin-4-yl)aniline (55 mg, 0.27 mmol), and the resulting reaction mixture was subjected to microwave irradiation at 60° C. for 1 h. After completion of the reaction (TLC and UPLC), the volatiles were removed under reduced pressure and the crude residue was neutralized with saturated aq. NaHCO₃ solution. The aqueous layer was extracted with CH₂Cl₂ (20 mL×3), and the combined organic layer was dried over Na₂SO₄, filtered and concentrated under reduced pressure. The resulting crude material was purified by flash chromatography (SiO₂/100-200 mesh; ~10% MeOH/CH₂Cl₂) to afford N-(2-chloro-6-(trifluoromethyl)phenyl)-4-methoxy-2-((3-methyl-4-(1-methylpiperidin-4-yl)phenyl)amino)pyrimidine-5-carboxamide (Compound 122, 34 mg, 25% yield) as an off-white solid. ¹H-NMR (400 MHz, DMSO-d₆): δδ 10.03 (s, 1H), 9.57 (s, 1H), 8.72 (s, 1H), 7.94 (d, J=7.60 Hz, 1H), 7.81 (dd, J=7.2, 0.8 Hz, 1H), 7.62-7.54 (m, 3H), 7.17 (d, J=8.4 Hz, 1H), 4.10 (s, 3H), 2.89-2.86 (m, 2H), 2.62-2.58 (m, 1H), 2.31 (s, 3H), 2.20 (s, 3H), 1.93-2.03 (m, 2H), 1.10-1.20 (m, 4H). Chemical Formula: C₂₆H₂₇ClF₃N₅O₂, Exact Mass: 533.18, Molecular Weight: 533.98. LCMS (ESI) m/z=534.2/536.0 (M+H), chlorine isotopes, t_{R}:1.571 min, 97.6% (Method-A). HPLC: 97.6%; t_{R}: 3.997 min (Method-A).

### Example 23. Synthesis of N-(2-chloro-6-ethynylphenyl)-4-methoxy-2-((3-methyl-4-(1-methylpiperidin-4-yl)phenyl)amino)pyrimidine-5-carboxamide (Compound 129)

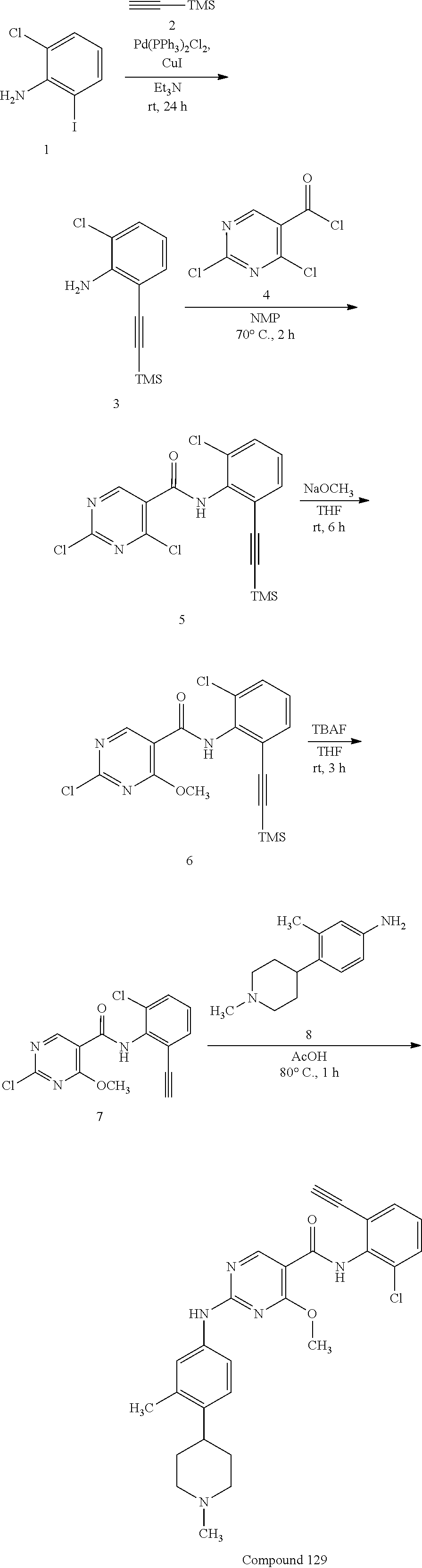

### 2-chloro-6-((trimethylsilyl)ethynyl)aniline

To a stirred solution of 2-chloro-6-iodoaniline (1 g, 3.95 mmol) in Et₃N (8 mL) were added bis(triphenylphosphine)palladium(II) dichloride (0.138 g, 0.197 mmol), copper(I) iodide (0.075 g, 0.395 mmol) and ethynyltrimethylsilane (0.765 mL, 5.52 mmol) at room temperature. The resulting reaction mixture was stirred at room temperature for 24 h. After completion of the reaction (TLC and UPLC), the mixture was filtered through celite, the filtrate was diluted with water, and extracted with ethyl acetate (30 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 2-chloro-6-((trimethylsilyl)ethynyl)aniline (820 mg, 91% yield) as a yellow solid. ¹H-NMR (400 MHz, CDCl₃): δ 7.23 (d, J=7.60 Hz, 2H), 6.61 (t, J=8.00 Hz, 1H), 4.64 (s, 2H), 0.29 (s, 9H). Chemical Formula: C₁₁H₁₄ClNSi, Exact Mass: 223.1, Molecular Weight: 223.8. LCMS (ESI) im/z=224.2/226.2/227.2 (M+H), chlorine isotopes, t_{R}=2.494 min, 98.6% (Method-A).

### 2,4-dichloro-N-(2-chloro-6-((trimethylsilyl)ethynyl)phenyl)pyrimidine-5-carboxamide

To a stirred solution of 2,4-dichloropyrimidine-5-carbonyl chloride (500 mg, 2.365 mmol) in NMP (5 mL), was added 2-chloro-6-((trimethylsilyl)ethynyl)aniline (635 mg, 2.84 mmol) at room temperature. The resulting reaction mixture was stirred at 70° C. for 2 h under nitrogen atmosphere. After completion of the reaction (TLC and UPLC), the reaction mixture was diluted with water and extracted with ethyl acetate (20 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 2,4-dichloro-N-(2-chloro-6-((trimethylsilyl)ethynyl)phenyl)pyrimidine-5-carboxamide (880 mg, 91% yield) as an off white solid. ¹H-NMR (400 MHz, DMSO-d₆): δ 10.80 (s, 1H), 8.92 (s, 1H), 7.66 (dd, J=1.20, 8.00 Hz, 1H), 7.57 (dd, J=1.20, 8.00 Hz, 1H), 7.41 (t, J=8.00 Hz, 1H), 0.21 (s, 9H). Chemical Formula: Cl₆H₁₄Cl₃N₃OSi, Exact Mass: 397.0, Molecular Weight: 398.7. LCMS (ESI) m/z=398.0 (M+H), t_{R}=2.253 min, 94.7% (Method-A).

### 2-chloro-N-(2-chloro-6-((trimethylsilyl)ethynyl)phenyl)-4-methoxypyrimidine-5-carboxamide

To a stirred solution of 2,4-dichloro-N-(2-chloro-6-((trimethylsilyl)ethynyl)phenyl) pyrimidine-5-carboxamide (880 mg, 2.207 mmol) in THF (10 mL) was added sodium methanolate (179 mg, 3.31 mmol) at 0° C. The reaction mixture was stirred at room temperature for 6 h. After completion of the reaction (TLC and UPLC), the reaction mixture was diluted with water and extracted with ethyl acetate (20 mL×2). The combined organic layer was dried over Na₂SO₄, filtered and concentrated under reduced pressure. The resulting crude material was purified by flash chromatography (SiO₂/100-200 mesh; 0-100% Ethyl acetate/Hexane) to afford 2-chloro-N-(2-chloro-6-((trimethylsilyl)ethynyl)phenyl)-4-methoxypyrimidine-5-carboxamide (570 mg, 49% yield) as an off white solid. Chemical Formula: C₁₇H₁₇Cl₂N₃O₂Si, Exact Mass: 393.0, Molecular Weight: 394.3. LCMS (ESI) m/z=394.2 (M+H), t_{R}=2.433 min, 97.8% (Method-A).

### 2-chloro-N-(2-chloro-6-ethynylphenyl)-4-methoxypyrimidine-5-carboxamide

To a stirred solution of 2-chloro-N-(2-chloro-6-((trimethylsilyl)ethynyl)phenyl)-4-methoxypyrimidine-5-carboxamide (470 mg, 1.192 mmol) in THF (10 mL) was added 1 M TBAF in THF (1.192 mL, 1.192 mmol) at 0° C. The reaction mixture was stirred at room temperature for 3 h. After completion of the reaction (TLC and UPLC), the reaction mixture was quenched with water extracted with ethyl acetate (30 mL×2). The combined organic layer was dried over Na₂SO₄, filtered and concentrated under reduced pressure. The resulting crude material was purified by flash chromatography (SiO₂/100-200 mesh; 0-100% Ethyl acetate/Hexane) to afford 2-chloro-N-(2-chloro-6-ethynylphenyl)-4-methoxypyrimidine-5-carboxamide (150 mg, 28% yield) as an off white solid. Chemical Formula: C₁₄H₉Cl₂N₃O₂, Exact Mass: 321.0, Molecular Weight: 322.1. LCMS (ESI) m/z=322.0/324.0 (M+H), chlorine isotopes, t_{R}=1.944 min, 71.7% (Method-A).

### N-(2-chloro-6-ethynylphenyl)-4-methoxy-2-((3-methyl-4-(1-methylpiperidin-4-yl)phenyl)amino)pyrimidine-5-carboxamide (Compound 129)

To a stirred solution of 2-chloro-N-(2-chloro-6-ethynylphenyl)-4-methoxypyrimidine-5-carboxamide (150 mg, 0.466 mmol) in acetic acid (5 mL) was added 3-methyl-4-(1-methylpiperidin-4-yl)aniline (124 mg, 0.605 mmol) at room temperature and was stirred at 80° C. for 1 h under microwave irradiation. After completion of the reaction (TLC and UPLC), the reaction mixture was concentrated under reduced pressure. The resultant crude was basified with aqueous sodium bicarbonate and extracted with ethyl acetate (20 mL×2). The combined organic layer was dried over Na₂SO₄, filtered and concentrated under reduced pressure. The resulting crude material was purified by flash chromatography (SiO₂/100-200 mesh; 0-10% methanol/CH₂Cl₂) to afford N-(2-chloro-6-ethynylphenyl)-4-methoxy-2-((3-methyl-4-(1-methylpiperidin-4-yl)phenyl)amino)pyrimidine-5-carboxamide (81 mg, 34% yield) as an off-white solid. ¹H-NMR (400 MHz, DMSO-d₆): δ 10.05 (s, 1H), 9.55 (s, 1H), 8.74 (s, 1H), 7.63-7.60 (m, 3H), 7.55 (dd, J=1.20, 7.60 Hz, 1H), 7.36 (t, J=8.00 Hz, 1H), 7.14 (d, J=8.80 Hz, 1H), 4.39 (s, 1H), 4.12 (s, 3H), 3.40-3.38 (m, 1H), 2.92-2.84 (m, 2H), 2.67 (s, 3H), 2.34 (s, 3H), 1.87-1.80 (m, 4H). Note: 2 protons merged in NMR solvent peak. Chemical Formula: C₂₇H₂₈ClN₅O₂, Exact Mass: 489.2, Molecular Weight: 490.0. LCMS (ESI) m/z=490.2 (M+H), t_{R}=1.555 min, 98.6% (Method-A). HPLC purity: 96.5%; t_{R}=3.899 min, (Method-A).

### Example 24. Synthesis of N-(2-chloro-6-methoxyphenyl)-4-methoxy-2-((3-methyl-4-(1-methylpiperidin-4-yl)phenyl)amino)pyrimidine-5-carboxamide (Compound 130)

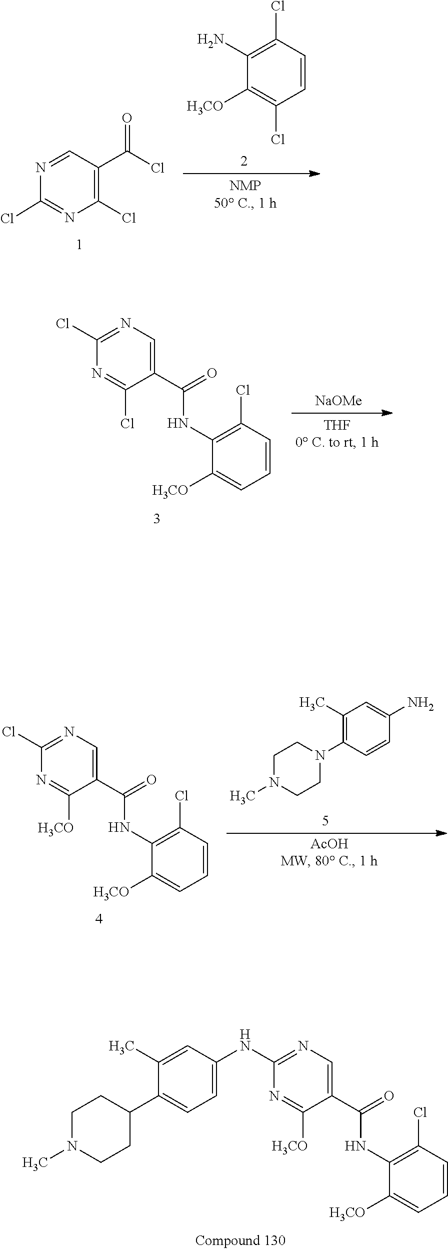

### 2,4-dichloro-N-(2-chloro-6-methoxyphenyl)pyrimidine-5-carboxamide

To a stirred solution of 2,4-dichloropyrimidine-5-carbonyl chloride (500 mg, 2.365 mmol) was added 2-chloro-6-methoxyaniline (373 mg, 2.365 mmol) and the reaction mixture was stirred at 50° C. for 1 h to afford 2,4-dichloro-N-(2-chloro-6-methoxyphenyl)pyrimidine-5-carboxamide (360 mg, 44% yield) as an off-white solid. ¹H-NMR (400 MHz, DMSO-d₆): δ 10.3 (s, 1H), 8.95 (s, 1H), 7.37 (t, J=8.0 Hz, 1H), 7.14 (dd, J=10.4, 8.4 Hz, 2H), 3.85 (s, 3H). Chemical Formula: C₁₂H₈Cl₃N₃O₂, Exact Mass: 330.97, Molecular Weight: 332.57. LCMS (ESI) m/z=332.0 (M+H), t_{R}=1.755 min, 97% (Method-A).

### 2-chloro-N-(2-chloro-6-methoxyphenyl)-4-methoxypyrimidine-5-carboxamide

To a stirred solution of 2,4-dichloro-N-(2-chloro-6-methoxyphenyl) pyrimidine-5-carboxamide (310 mg, 0.932 mmol) was added sodium methanolate (151 mg, 2.80 mmol) and the reaction mixture was stirred at room temperature for 1 h to afford 2-chloro-N-(2-chloro-6-methoxyphenyl)-4-methoxypyrimidine-5-carboxamide (220 mg, 65% yield) as an off white solid. ¹H-NMR (400 MHz, DMSO-d₆): δ 10.3 (s, 1H), 8.96 (s, 1H), 7.35 (t, J=8.4 Hz, 1H), 7.17-7.10 (m, 2H), 4.10 (s, 3H), 3.90 (s, 3H). Chemical Formula: C₁₃H₁₁Cl₂N₃O₃, Exact Mass: 327.02, Molecular Weight: 328.15. LCMS (ESI) m/z=328.0/330.0/331.0 (M+H), chlorine isotopes, t_{R}=1.796 min, 90.4% (Method-A).

### N-(2-chloro-6-methoxyphenyl)-4-methoxy-2-((3-methyl-4-(1-methylpiperidin-4-yl)phenyl)amino)pyrimidine-5-carboxamide (Compound 130)

To a stirred solution of 2-chloro-N-(2-chloro-6-methoxyphenyl)-4-methoxy pyrimidine-5-carboxamide (150 mg, 0.457 mmol) was added 3-methyl-4-(1-methylpiperidin-4-yl)aniline (93 mg, 0.457 mmol) and the reaction mixture was stirred at 80° C. for 1 h under microwave irradiation. The resultant crude residue was purified by prep. HPLC to afford N-(2-chloro-6-methoxyphenyl)-4-methoxy-2-((3-methyl-4-(1-methylpiperidin-4-yl)phenyl)amino) pyrimidine-5-carboxamide (Compound 130, 12 mg, 5% yield) as an off-white solid. ¹H-NMR (400 MHz, DMSO-d₆): δ 9.98 (s, 1H), 9.08 (s, 1H), 8.73 (s, 1H), 7.58-7.55 (m, 2H), 7.32 (t, J=8.4 Hz, 1H), 7.17-7.08 (m, 3H), 4.11 (s, 3H), 3.80 (s, 3H), 2.89-2.86 (m, 2H), 2.34 (s, 3H), 2.21 (s, 3H), 2.01-1.95 (m, 2H), 1.67-1.64 (m, 4H). Note: One proton merged in NMR solvent. Chemical Formula: C₂₆H₃₀ClN₅O₃, Exact Mass: 495.20, Molecular Weight: 496.01. LCMS (ESI) m/z=496.2/497.2 (M+H), Chlorine isotopes, t_{R}=1.541 min, 98.3% (Method-A). HPLC: 98.2%; t_{R}: 3.747 min (Method-A).

### Example 25. Synthesis of N-(2-chloro-5-cyanophenyl)-4-methoxy-2-((3-methyl-4-(1-methylpiperidin-4-yl)phenyl)amino)pyrimidine-5-carboxamide (Compound 131)

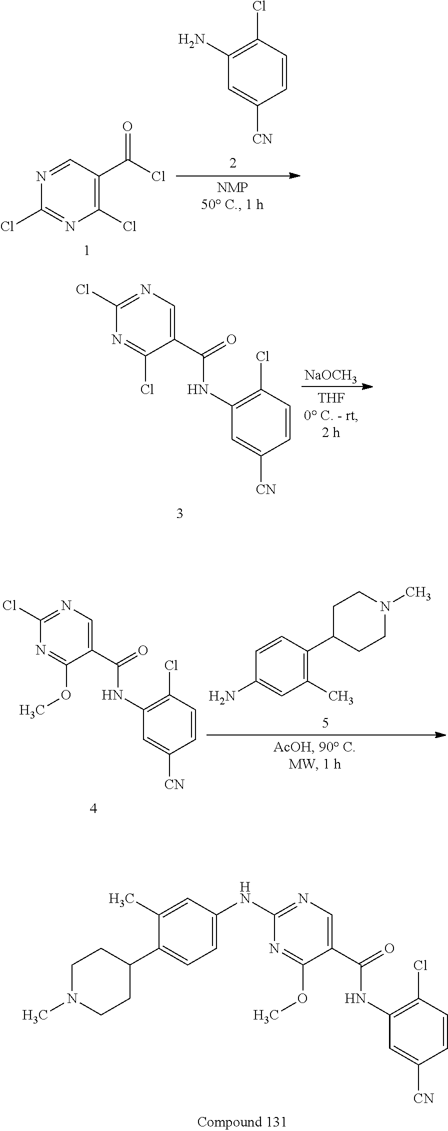

### 2,4-dichloro-N-(2-chloro-5-cyanophenyl)pyrimidine-5-carboxamide

To a stirred solution of 2,4-dichloropyrimidine-5-carbonyl chloride (500 mg, 2.365 mmol) was added 3-amino-4-chlorobenzonitrile (361 mg, 2.365 mmol) and the reaction mixture was stirred at 50° C. for 1 h to afford 2,4-dichloro-N-(2-chloro-5-cyanophenyl)pyrimidine-5-carboxamide (700 mg, 90% yield) as a yellow solid. ¹H-NMR (400 MHz, DMSO-d₆): δ 10.77 (s, 1H), 9.09 (s, 1H), 8.34 (d, J=1.60 Hz, 1H), 7.86-7.79 (m, 2H). Chemical Formula: C₁₂H₅Cl₃N₄O, Exact Mass: 326.0, Molecular Weight: 327.5. LCMS (ESI) m/z=327.0 (M−H), t_{R}=1.958 min, 80.7% (Method-A).

### 2-chloro-N-(2-chloro-5-cyanophenyl)-4-methoxypyrimidine-5-carboxamide

To a stirred solution of 2,4-dichloro-N-(2-chloro-5-cyanophenyl)pyrimidine-5-carboxamide (300 mg, 0.916 mmol) was added sodium methoxide (49.5 mg, 0.916 mmol) and the reaction mixture was stirred at room temperature for 2 h to afford 2-chloro-N-(2-chloro-5-cyanophenyl)-4-methoxypyrimidine-5-carboxamide (90 mg, 0.279 mmol, 30.4% yield) as an off white solid. ¹H-NMR (400 MHz, DMSO-d₆): δ 10.23 (s, 1H), 8.95 (s, 1H), 8.59 (d, J=1.60 Hz, 1H), 7.86 (d, J=8.40 Hz, 1H), 7.75 (dd, J=2.00, 8.40 Hz, 1H), 4.17 (s, 3H). Chemical Formula: C₁₃H₈Cl₂N₄O₂, Exact Mass: 322.0, Molecular Weight: 323.1. LCMS (ESI) m/z=323.0/325.0 (M+H), Chlorine isotopes, t_{R}=2.029 min, 99.4% (Method-A). Note: Regioselectivity was confirmed by 1D nOe.

### N-(2-chloro-5-cyanophenyl)-4-methoxy-2-((3-methyl-4-(1-methylpiperidin-4-yl)phenyl)amino)pyrimidine-5-carboxamide (Compound 131)

To a stirred solution of 2-chloro-N-(2-chloro-5-cyanophenyl)-4-methoxypyrimidine-5-carboxamide (80 mg, 0.248 mmol) was added 3-methyl-4-(1-methylpiperidin-4-yl)aniline (50.6 mg, 0.248 mmol) and the reaction mixture was stirred at 90° C. for 1 h under microwave irradiation to afford N-(2-chloro-5-cyanophenyl)-4-methoxy-2-((3-methyl-4-(1-methylpiperidin-4-yl)phenyl)amino)pyrimidine-5-carboxamide (Compound 131, 35 mg, 29% yield) as an off white solid. ¹H-NMR (400 MHz, DMSO-d₆): δ 10.19 (s, 1H), 10.10 (s, 1H), 8.87 (s, 1H), 8.84 (d, J=1.60 Hz, 1H), 7.83 (d, J=8.40 Hz, 1H), 7.64 (dd, J=1.60, 8.20 Hz, 1H), 7.59-7.54 (m, 2H), 7.18 (d, J=8.80 Hz, 1H), 4.21 (s, 3H), 2.89-2.86 (m, 2H), 2.62-2.58 (m, 1H), 2.29 (s, 3H), 2.20 (s, 3H), 2.01-1.95 (m, 2H), 1.68-1.60 (m, 4H). Chemical Formula: C₂₆H₂₇ClN₆O₂, Exact Mass: 490.2, Molecular Weight: 491.0. LCMS (ESI) m/z=491.2/492.2 (M+H), chlorine isotopes, t_{R}=1.720 min, 99.6% (Method-A), HPLC purity: 99.7%; t_{R}=4.300 min (Method-A).

### Example 26. Synthesis of N-(2-chloro-6-ethylphenyl)-4-methoxy-2-((3-methyl-4-(1-methylpiperidin-4-yl)phenyl)amino)pyrimidine-5-carboxamide (Compound 132)

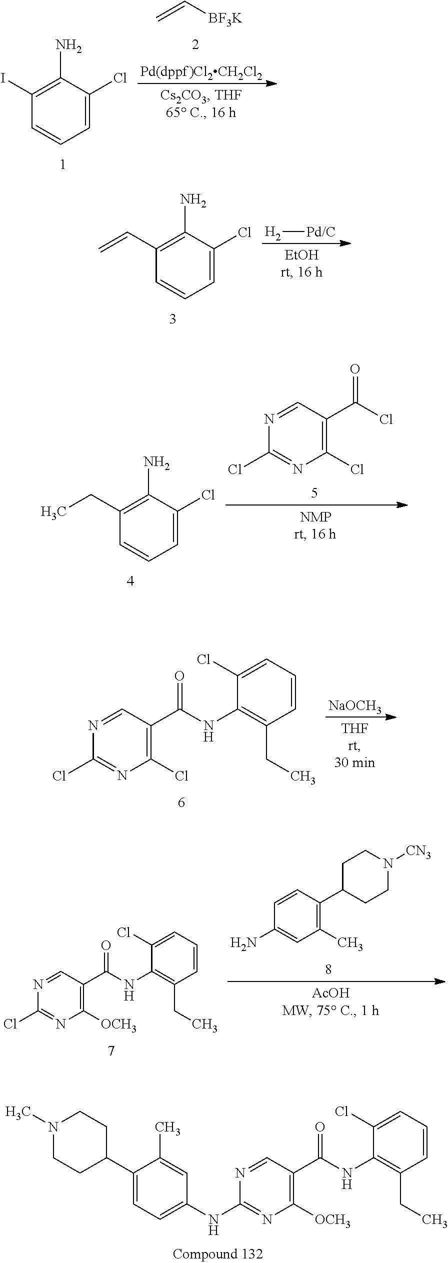

### 2-chloro-6-vinylaniline

To a stirred solution of 2-chloro-6-iodoaniline (2.0 g, 7.89 mmol) and potassium vinyl trifluoroborate (3.09 g, 9.47 mmol) in THF (25 mL) was added cesium carbonate (5.14 g, 15.78 mmol) at room temperature. After degassing with nitrogen for 5 min, PdCl₂(dppf)·CH₂Cl₂ (0.644 g, 0.789 mmol) was added to the reaction mixture under nitrogen atmosphere. The resulting reaction mixture was stirred at 65° C. for 16 h. After completion of the reaction (TLC and UPLC), the reaction mixture was filtered through celite, the filtrate was diluted with water, and extracted with ethyl acetate (30 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 2-chloro-6-vinylaniline (800 mg, 66% yield) as a yellow solid. ¹H-NMR (400 MHz, DMSO-d₆): δ 7.28 (dd, J=1.20, 7.60 Hz, 1H), 7.17 (dd, J=1.20, 7.80 Hz, 1H), 7.00-6.93 (m, 1H), 6.59-6.55 (m, 1H), 5.65 (dd, J=1.60, 17.20 Hz, 1H), 5.31 (s, 2H), 5.24 (dd, J=1.20, 11.00 Hz, 1H). Chemical Formula: C₈H₈ClN, Exact Mass: 153.0, Molecular Weight: 153.6.

### 2-chloro-6-ethylaniline

To a stirred solution of 2-chloro-6-vinylaniline (700 mg, 4.56 mmol) in ethanol (20 mL), was added 10% palladium on carbon (582 mg, 5.47 mmol) at room temperature. The resulting reaction mixture was stirred at room temperature for 16 h under hydrogen atmosphere. After completion of the reaction (TLC and UPLC), the reaction mixture was filtered through celite and filtrate was concentrated under reduced pressure. The resulting crude material was purified by flash chromatography (SiO₂/100-200 mesh; 0-100% EtOAc/hexanes) to afford 2-chloro-6-ethylaniline (450 mg, 63% yield) as a yellow solid. ¹H-NMR (400 MHz, DMSO-d₆): δ 7.07 (dd, J=1.60, 8.00 Hz, 1H), 6.94-6.92 (m, 1H), 6.53 (t, J=7.60 Hz, 1H), 5.00 (s, 2H), 2.55-2.49 (m, 2H), 1.15-1.11 (m, 3H). Chemical Formula: C₈H₁₀ClN, Exact Mass: 155.1, Molecular Weight: 155.6

### 2,4-dichloro-N-(2-chloro-6-ethylphenyl)pyrimidine-5-carboxamide

To a stirred solution of 2,4-dichloropyrimidine-5-carbonyl chloride (0.426 ml, 3.31 mmol) in N-methyl-2-pyrrolidone (10 mL) was added 2-chloro-6-ethylaniline (0.450 g, 2.89 mmol) at room temperature. The reaction mixture was stirred at room temperature for 16 h. After completion of the reaction (TLC and UPLC), the reaction mixture was diluted with water and extracted with ethyl acetate (20 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 2,4-dichloro-N-(2-chloro-6-ethylphenyl)pyrimidine-5-carboxamide (350 mg, 32% yield) as an off white solid. ¹H-NMR (400 MHz, DMSO-d₆): δ 10.48 (s, 1H), 9.03 (s, 1H), 7.45 (dd, J=2.80, 7.00 Hz, 1H), 7.38-7.33 (m, 2H), 2.68 (q, J=7.60 Hz, 2H), 1.17 (t, J=7.60 Hz, 3H). Chemical Formula: C₁₃H₁₀Cl₃N₃O, Exact Mass: 329.0, Molecular Weight: 330.6. LCMS (ESI) m/z=328.0/330.0 (M−H), Chlorine isotopes, t_{R}=2.071 min, 99.1% (Method-A).

### 2-chloro-N-(2-chloro-6-ethylphenyl)-4-methoxypyrimidine-5-carboxamide

To a stirred solution of 2,4-dichloro-N-(2-chloro-6-ethylphenyl)pyrimidine-5-carboxamide (350 mg, 1.059 mmol) in THF (10 mL) was added sodium methoxide (114 mg, 2.117 mmol) at 0° C. The reaction mixture was stirred at room temperature for 30 minutes. After completion of the reaction (TLC and UPLC), the reaction mixture was diluted with water and extracted with ethyl acetate (20 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 2-chloro-N-(2-chloro-6-ethylphenyl)-4-methoxypyrimidine-5-carboxamide (140 mg, 40% yield) as an off-white solid. ¹H-NMR (400 MHz, CDCl₃): δ 9.25 (s, 1H), 8.82 (s, 1H), 7.38-7.36 (m, 1H), 7.28-7.26 (m, 2H), 4.31 (s, 3H), 2.69 (q, J=7.60 Hz, 2H), 1.24 (t, J=7.60 Hz, 3H). Chemical Formula: C₁₄H₁₃Cl₂N₃O₂, Exact Mass: 325.0, Molecular Weight: 326.2. Note: Regioselectivity was confirmed by 1D nOe.

### N-(2-chloro-6-ethylphenyl)-4-methoxy-2-((3-methyl-4-(I-methylpiperidin-4-yl)phenyl)amino)pyrimidine-5-carboxamide (Compound 132)

To a stirred solution of 2-chloro-N-(2-chloro-6-ethylphenyl)-4-methoxypyrimidine-5-carboxamide (130 mg, 0.399 mmol) in acetic acid (5 mL) was added 3-methyl-4-(1-methylpiperidin-4-yl)aniline (81 mg, 0.399 mmol) at room temperature and was stirred at 75° C. for 1 h under microwave irradiation. After completion of the reaction (TLC and UPLC), the reaction mixture was concentrated under reduced pressure. The resultant crude was basified with 10% aqueous sodium bicarbonate and extracted with ethyl acetate (20 mL×2). The combined organic layer was dried over Na₂SO₄, filtered and concentrated under reduced pressure. The resulting crude material was purified by flash chromatography (SiO₂/100-200 mesh; 0-10% Methanol/CH₂Cl₂) to N-(2-chloro-6-ethylphenyl)-4-methoxy-2-((3-methyl-4-(1-methylpiperidin-4-yl)phenyl)amino) pyrimidine-5-carboxamide (14 mg, 7% yield) as an off-white solid. ¹H-NMR (400 MHz, DMSO-d₆): δ 9.97 (s, 1H), 9.36 (s, 1H), 8.71 (d, J=2.80 Hz, 1H), 7.58-7.55 (m, 2H), 7.42-7.38 (m, 1H), 7.33-7.29 (m, 2H), 7.16 (d, J=8.00 Hz, 1H), 4.11 (s, 3H), 2.90-2.87 (m, 2H), 2.64-2.58 (m, 3H), 2.29 (s, 3H), 2.21 (s, 3H), 2.03-1.97 (m, 2H), 1.67-1.63 (m, 4H), 1.14 (t, J=7.60 Hz, 3H). Chemical Formula: C₂₇H₃₂C₁N₅O₂, Exact Mass: 493.2, Molecular Weight: 494.0. LCMS (ESI) m/z=494.4/495.1/496.0 (M+H), chlorine isotopes, t_{R}=2.080 min, 99.2% (Method-B), HPLC purity: 98.6%; t_{R}=4.101 min, (Method-A).

### Example 27. Synthesis of N-(2-bromo-6-fluorophenyl)-4-methoxy-2-((3-methyl-4-(1-methylpiperidin-4-yl)phenyl)amino)pyrimidine-5-carboxamide (Compound 136)

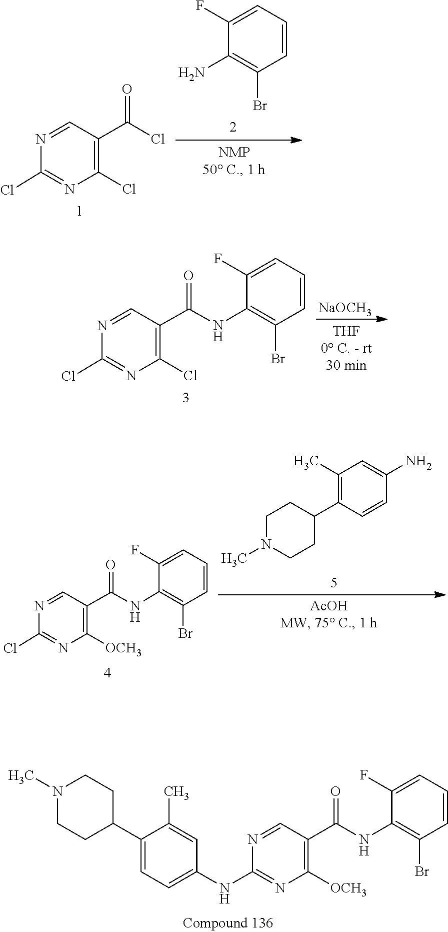

### N-(2-bromo-6-fluorophenyl)-2,4-dichloropyrimidine-5-carboxamide

To a stirred solution of 2,4-dichloropyrimidine-5-carbonyl chloride (0.365 mL, 2.84 mmol) was added 2-bromo-6-fluoroaniline (0.322 mL, 2.84 mmol) and the reaction mixture was stirred at 50° C. for 1 h to afford N-(2-bromo-6-fluorophenyl)-2,4-dichloropyrimidine-5-carboxamide (500 mg, 40% yield) as a brown solid. ¹H-NMR (400 MHz, DMSO-d₆): δ 10.70 (s, 1H), 9.03 (s, 1H), 7.64-7.62 (m, 1H), 7.46-7.39 (m, 2H). Chemical Formula: C₁₁H₅BrCl₂FN₃O, Exact Mass: 362.9, Molecular Weight: 365.0. LCMS (ESI) m/z=364.0/364.8/365.8 (M−H), chlorine isotopes, t_{R}=1.891 min, 83.8% (Method-A).

### N-(2-bromo-6-fluorophenyl)-2-chloro-4-methoxypyrimidine-5-carboxamide

To a stirred solution of N-(2-bromo-6-fluorophenyl)-2,4-dichloro-4,5-dihydropyrimidine-5-carboxamide (360 mg, 0.981 mmol) was added NaOCH₃ (148 mg, 2.75 mmol) and the reaction mixture was stirred at 0° C. for 30 minutes to afford N-(2-bromo-6-fluorophenyl)-2-chloro-4-methoxypyrimidine-5-carboxamide (170 mg, 39% yield) as an off-white solid. ¹H-NMR (400 MHz, DMSO-d₆): δ 10.02 (s, 1H), 8.83 (s, 1H), 7.61 (dd, J=2.00, 6.20 Hz, 1H), 7.42-7.34 (m, 2H), 4.11 (s, 3H). Chemical Formula: C₁₂H₈BrClFN₃O₂, Exact Mass: 358.9, Molecular Weight: 360.6. LCMS (ESI) m/z=360.0/362.0 (M+H), Chlorine isotopes, t_{R}=1.958 min, 81.6% (Method-A). Note: Regioselectivity was confirmed by 1D nOe.

### N-(2-bromo-6-fluorophenyl)-4-methoxy-2-((3-methyl-4-(I-methylpiperidin-4-yl)phenyl)amino)pyrimidine-5-carboxamide (Compound 136)

To a stirred solution of N-(2-bromo-6-fluorophenyl)-2-chloro-4-methoxypyrimidine-5-carboxamide (160 mg, 0.444 mmol) was added 3-methyl-4-(1-methylpiperidin-4-yl)aniline (254 mg, 1.242 mmol) and the reaction mixture was stirred at 75° C. for 1 h under microwave irradiation to afford N-(2-bromo-6-fluorophenyl)-4-methoxy-2-((3-methyl-4-(1-methylpiperidin-4-yl)phenyl) amino)pyrimidine-5-carboxamide (100 mg, 42% yield) as an off white solid. ¹H-NMR (400 MHz, DMSO-d₆): δ 10.02 (s, 1H), 9.42 (s, 1H), 8.75 (s, 1H), 7.62-7.57 (m, 3H), 7.39-7.32 (m, 2H), 7.14 (d, J=8.80 Hz, 1H), 4.12 (s, 3H), 3.40-3.30 (m, 2H), 3.00-2.88 (m, 3H), 2.68-2.67 (m, 3H), 2.32 (s, 3H), 1.90-1.80 (m, 4H). Chemical Formula: C₂₅H₂₇BrFN₅O₂, Exact Mass: 527.1, Molecular Weight: 528.4. LCMS (ESI) m/z=528.2/531.2 (M+H), bromine isotopes, t_{R}=1.490 min, 99.4% (Method-A), HPLC purity: 99.1%; t_{R}=3.864 min (Method-A).

### Example 28. Synthesis of N-(2-chloro-6-cyanophenyl)-4-methoxy-2-((3-methyl-4-(1-methylpiperidin-4-yl)phenyl)amino)pyrimidine-5-carboxamide (Compound 137)

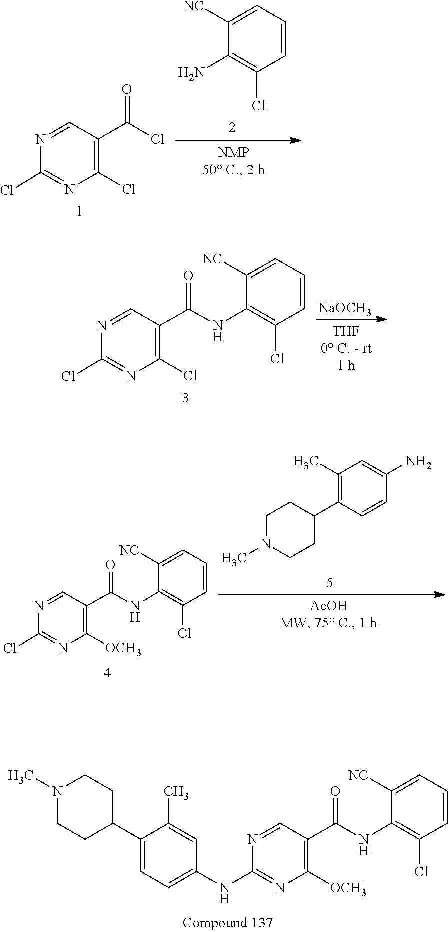

### 2,4-dichloro-N-(2-chloro-6-cyanophenyl)pyrimidine-5-carboxamide

To a stirred solution of 2,4-dichloropyrimidine-5-carbonyl chloride (1. g, 4.73 mmol) was added 2-amino-3-chlorobenzonitrile (0.722 g, 4.73 mmol) the reaction mixture was stirred at 50° C. for 2 h to afford 2,4-dichloro-N-(2-chloro-6-cyanophenyl)pyrimidine-5-carboxamide (1 g, 59% yield) as an off-white solid. ¹H-NMR (400 MHz, DMSO-d₆): δ 9.17 (s, 1H), 8.92 (s, 1H), 8.52 (dd, J=2.80, 6.60 Hz, 1H), 7.68 (dd, J=3.60, 6.20 Hz, 2H). Chemical Formula: C₁₂H₅Cl₃N₄O, Exact Mass: 326.0, Molecular Weight: 327.5. LCMS (ESI) m/z=324.8 (M−H), t_{R}=1.781 min, 91.8% (Method-B).

### 2-chloro-N-(2-chloro-6-cyanophenyl)-4-methoxypyrimidine-5-carboxamide

To a stirred solution of 2,4-dichloro-N-(2-chloro-6-cyanophenyl)pyrimidine-5-carboxamide (600 mg, 1.832 mmol) was added sodium methoxide (277 mg, 5.13 mmol) and the reaction mixture was stirred at 0° C. for 1 h to afford 2-chloro-N-(2-chloro-6-cyanophenyl)-4-methoxypyrimidine-5-carboxamide (100 mg, 17% yield) as an off-white solid. ¹H-NMR (400 MHz, DMSO-d₆): δ 10.50 (s, 1H), 8.83 (s, 1H), 8.00-7.95 (m, 2H), 7.59 (t, J=8.00 Hz, 1H), 4.12 (s, 3H). Chemical Formula: C₁₃H₈Cl₂N₄O₂, Exact Mass: 322.0, Molecular Weight: 323.1. LCMS (ESI) m/z=323.0/325.0/326.0 (M+H), Chlorine isotopes, t_{R}=1.839 min, 87.6% (Method-A). Note: Regioselectivity was confirmed by 1D nOe.

### N-(2-chloro-6-cyanophenyl)-4-methoxy-2-((3-methyl-4-(1-methylpiperidin-4-yl)phenyl)amino)pyrimidine-5-carboxamide (Compound 137)

To a stirred solution of 2-chloro-N-(2-chloro-6-cyanophenyl)-4-methoxypyrimidine-5-carboxamide (100 mg, 0.309 mmol) was added 3-methyl-4-(1-methylpiperidin-4-yl)aniline (63.2 mg, 0.309 mmol) and the reaction mixture was stirred at 75° C. for 1 h under microwave irradiation. The resultant crude residue was purified by prep. HPLC to get N-(2-chloro-6-cyanophenyl)-4-methoxy-2-((3-methyl-4-(1-methylpiperidin-4-yl)phenyl)amino)pyrimidine-5-carboxamide (14.3 mg, 9% yield) as an off-white solid. ¹H-NMR (400 MHz, DMSO-d₆): δ 10.09 (s, 1H), 9.86 (s, 1H), 8.77 (s, 1H), 8.35 (s, 1H), 7.97-7.91 (m, 2H), 7.58-7.52 (m, 3H), 7.18-7.15 (m, 1H), 4.13 (s, 3H), 2.90-2.87 (m, 2H), 2.62-2.58 (m, 1H), 2.30 (s, 3H), 2.21 (s, 3H), 2.03-1.97 (m, 2H), 1.68-1.60 (m, 4H). Note: One extra proton for HCOOH salt. Chemical Formula: C₂₆H₂₇ClN₆O₂, Exact Mass: 490.2, Molecular Weight: 491.0. LCMS (ESI) m/z=491.2/493.2/494.2 (M+H), chlorine isotopes, t_{R}=1.501 min, 96.2% (Method-A), HPLC purity: 96.2%; t_{R}=3.662 min (Method-A).

### Example 29. Synthesis of N-(2,6-dichloro-3-hydroxyphenyl)-4-methoxy-2-((3-methyl-4-(1-methylpiperidin-4-yl)phenyl)amino)pyrimidine-5-carboxamide (Compound 133)

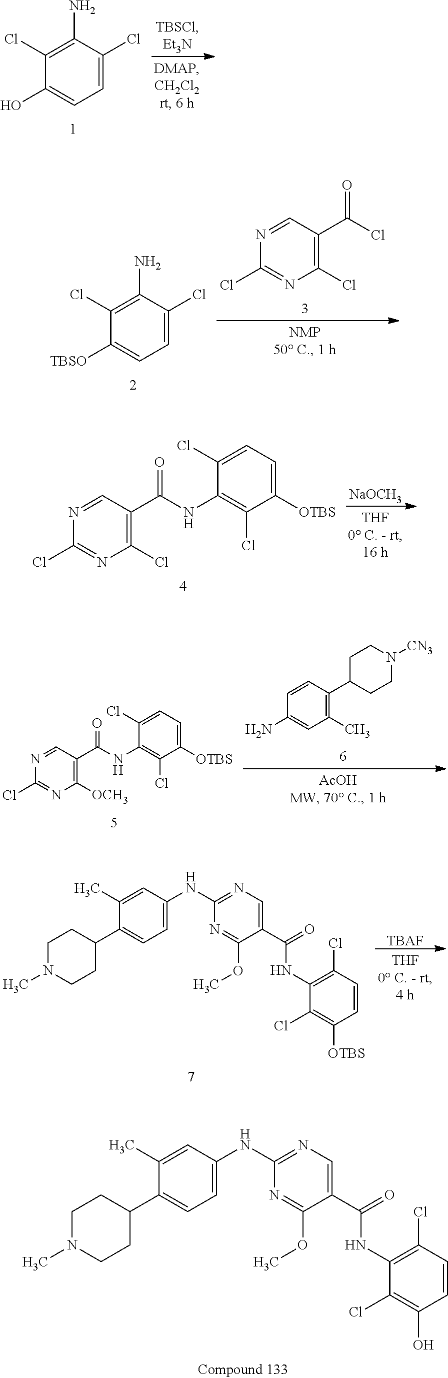

### 3-((tert-butyldimethylsilyl)oxy)-2,6-dichloroaniline

To a stirred solution of 3-amino-2,4-dichlorophenol (500 mg, 2.81 mmol) was added TBS Chloride (1270 mg, 8.43 mmol) as reactants and the reaction mixture was stirred at room temperature for 6 h to afford 3-((tert-butyldimethylsilyl)oxy)-2,6-dichloroaniline (450 mg, 55% yield) as a yellow liquid. ¹H-NMR (400 MHz, DMSO-d₆): δ 7.10 (m, 1H), 6.29-6.25 (m, 1H), 5.44 (s, 2H), 0.99 (s, 9H), 0.20 (s, 6H). Chemical Formula: C₁₂H₁₉Cl₂NOSi, Exact Mass: 291.1, Molecular Weight: 292.3. LCMS (ESI) m/z=292.2/294.2 (M+H), Chlorine isotopes, t_{R}=2.841 min, 99.5% (Method-A).

### N-(3-((tert-butyldimethylsilyl)oxy)-2,6-dichlorophenyl)-2,4-dichloropyrimidine-5-carboxamide

To a stirred solution of 3-((tert-butyldimethylsilyl)oxy)-2,6-dichloroaniline (456 mg, 1.561 mmol) was added 2,4-dichloropyrimidine-5-carbonyl chloride (0.201 mL, 1.561 mmol) and the reaction mixture was stirred at 50° C. for 1 h to afford N-(3-((tert-butyldimethylsilyl)oxy)-2,6-dichlorophenyl)-2,4-dichloropyrimidine-5-carboxamide (400 mg, 55% yield) as an off white solid. ¹H-NMR (400 MHz, DMSO-d₆): δ 10.81 (s, 1H), 9.01 (s, 1H), 7.48 (d, J=8.80 Hz, 1H), 7.11 (d, J=9.20 Hz, 1H), 1.02 (s, 9H), 0.27 (s, 6H). Chemical Formula: C₁₇H₁₉Cl₄N₃O₂Si, Exact Mass: 465.0, Molecular Weight: 467.2. LCMS (ESI) m/z=466.0/468.0/469.0 (M+H), Chlorine isotopes, t_{R}=2.681 min, 93.6% (Method-A).

### N-(3-((tert-butyldimethylsilyl)oxy)-2,6-dichlorophenyl)-2-chloro-4-methoxypyrimidine-5-carboxamide

To a stirred solution of N-(3-((tert-butyldimethylsilyl)oxy)-2,6-dichlorophenyl)-2,4-dichloropyrimidine-5-carboxamide (350 mg, 0.749 mmol) in THF (10 mL) was added sodium methanolate (40.5 mg, 0.749 mmol) at 0° C. The reaction mixture was stirred at room temperature for 16 h. After completion of the reaction (TLC and UPLC), the reaction mixture was diluted with water and extracted with ethyl acetate (20 mL×2). The combined organic layer was dried over Na₂SO₄, filtered and concentrated under reduced pressure. The resulting crude material was purified by flash chromatography (SiO₂/100-200 mesh; 0-100% Ethyl acetate/Hexane) to afford N-(3-((tert-butyldimethylsilyl)oxy)-2,6-dichlorophenyl)-2-chloro-4-methoxypyrimidine-5-carboxamide (150 mg, 43% yield) as an off white solid. ¹H-NMR (400 MHz, DMSO-d₆): δ 10.15 (s, 1H), 8.81 (s, 1H), 7.46 (d, J=8.80 Hz, 1H), 7.08 (d, J=9.20 Hz, 1H), 4.09 (s, 3H), 1.01 (s, 9H), 0.26 (s, 6H). Chemical Formula: C₁₈H₂₂Cl₃N₃O₃Si, Exact Mass: 461.0, Molecular Weight: 462.8. LCMS (ESI) m/z=462.0/464.0/466.0 (M+H), Chlorine isotopes, t_{R}=2.751 min, 99.6% (Method-A). Note: Regioselectivity was confirmed by 1D nOe.

### N-(3-((tert-butyldimethylsilyl)oxy)-2,6-dichlorophenyl)-4-methoxy-2-((3-methyl-4-(1-methylpiperidin-4-yl)phenyl)amino)pyrimidine-5-carboxamide

To a stirred solution of N-(3-((tert-butyldimethylsilyl)oxy)-2,6-dichlorophenyl)-2-chloro-4-methoxypyrimidine-5-carboxamide (120 mg, 0.259 mmol) was added 3-methyl-4-(1-methylpiperidin-4-yl)aniline (53.0 mg, 0.259 mmol) and the reaction mixture was stirred at 70° C. for 1 h under microwave irradiation to afford N-(3-((tert-butyldimethylsilyl)oxy)-2,6-dichlorophenyl)-4-methoxy-2-((3-methyl-4-(1-methyl piperidin-4-yl)phenyl)amino)pyrimidine-5-carboxamide (80 mg, 49% yield) as an off-white solid. Chemical Formula: C₃₁H₄₁Cl₂N₅O₃Si, Exact Mass: 629.2, Molecular Weight: 630.7. LCMS (ESI) m/z=631.2/632.2/634.2 (M+H), Chlorine isotopes, t_{R}=2.116 min, 70.4% (Method-A).

### N-(2,6-dichloro-3-hydroxyphenyl)-4-methoxy-2-((3-methyl-4-(I-methylpiperidin-4-yl)phenyl)amino)pyrimidine-5-carboxamide (Compound 133)

To a stirred solution of N-(3-((tert-butyldimethylsilyl)oxy)-2,6-dichlorophenyl)-4-methoxy-2-((3-methyl-4-(1-methyl piperidin-4-yl)phenyl)amino)pyrimidine-5-carboxamide (70 mg, 0.111 mmol) in THF (10 mL) was added 1M TBAF in THF (0.166 mL, 0.166 mmol) at 0° C. The reaction mixture was stirred at room temperature for 4 h. After completion of the reaction (TLC and UPLC), the reaction mixture was diluted with saturated aqueous ammonium chloride solution and extracted with 10% methanol in CH₂Cl₂ (20 mL×2). The combined organic layer was dried over Na₂SO₄, filtered and concentrated under reduced pressure. The resulting crude material was purified by flash chromatography (SiO₂/100-200 mesh; 0-10% methanol/CH₂Cl₂) to afford N-(2,6-dichloro-3-hydroxyphenyl)-4-methoxy-2-((3-methyl-4-(1-methylpiperidin-4-yl)phenyl)amino)pyrimidine-5-carboxamide (30 mg, 52% yield) as an off white solid. ¹H-NMR (400 MHz, DMSO-d₆): δ 10.00 (s, 1H), 9.45 (s, 1H), 8.73 (s, 1H), 7.58-7.54 (m, 2H), 7.33 (d, J=9.20 Hz, 1H), 7.16 (d, J=8.40 Hz, 1H), 6.97 (d, J=8.80 Hz, 1H), 4.11 (s, 3H), 2.89-2.86 (m, 2H), 2.61-2.56 (m, 2H), 2.29 (s, 3H), 2.20 (s, 3H), 1.67-1.64 (m, 2H), 1.68-1.60 (m, 4H). Chemical Formula: C₂₅H₂₇Cl₂N₅O₃, Exact Mass: 515.1, Molecular Weight: 516.4. LCMS (ESI) m/z=516.2/518.2/520.2 (M+H), Chlorine isotopes, t_{R}=1.436 min, 96.1% (Method-A), HPLC purity: 97.3%; t_{R}=3.541 min (Method-A).

### Example 30. Synthesis of N-(2-bromo-6-cyanophenyl)-4-methoxy-2-((3-methyl-4-(1-methylpiperidin-4-yl)phenyl)amino)pyrimidine-5-carboxamide (Compound 134)

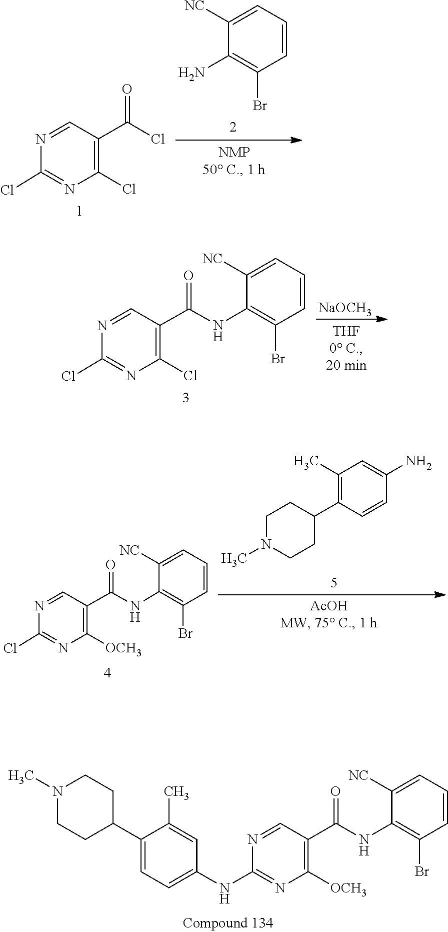

### N-(2-bromo-6-cyanophenyl)-2,4-dichloropyrimidine-5-carboxamide

To a stirred solution of 2,4-dichloropyrimidine-5-carbonyl chloride (0.40 g, 1.892 mmol) was added 2-amino-3-bromobenzonitrile (0.373 g, 1.892 mmol) the reaction mixture was stirred at 50° C. for 1 h to afford N-(2-bromo-6-cyanophenyl)-2,4-dichloropyrimidine-5-carboxamide (450 mg, 64% yield) as an off-white solid. ¹H-NMR (400 MHz, DMSO-d₆): δ 11.23 (s, 1H), 8.97 (s, 1H), 8.16 (dd, J=1.20, 8.40 Hz, 1H), 8.03 (dd, J=1.20, 7.80 Hz, 1H), 7.54 (t, J=8.00 Hz, 1H). Chemical Formula: C₁₂H₅BrCl₂N₄O, Exact Mass: 369.9, Molecular Weight: 372.0. LCMS (ESI) m/z=371.0/373.0 (M+H), chlorine and bromine isotopes, t_{R}=1.747 min, 96.1% (Method-A).

### N-(2-bromo-6-cyanophenyl)-2-chloro-4-methoxypyrimidine-5-carboxamide

To a stirred solution of N-(2-bromo-6-cyanophenyl)-2,4-dichloropyrimidine-5-carboxamide (0.30 g, 0.806 mmol) was added sodium methoxide (0.131 g, 2.419 mmol) and the reaction mixture was stirred at 0° C. for 20 minutes to afford N-(2-bromo-6-cyanophenyl)-2-chloro-4-methoxypyrimidine-5-carboxamide (0.080 g, 27% yield) as an off-white solid. ¹H-NMR (400 MHz, DMSO-d₆): δ 10.50 (s, 1H), 8.83 (s, 1H), 8.13 (dd, J=1.20, 8.00 Hz, 1H), 8.00 (dd, J=1.20, 7.60 Hz, 1H), 7.51 (t, J=8.00 Hz, 1H), 4.12 (s, 3H). Chemical Formula: C₁₃HsBrClN₄O₂, Exact Mass: 366.0, Molecular Weight: 367.6. LCMS (ESI) m/z=366.9/368.9/369.8/370.8 (M+H), chlorine and bromine isotopes, t_{R}=1.728 min, 97.6% (Method-A). Note: Regioselectivity was confirmed by 1D nOe.

### N-(2-bromo-6-cyanophenyl)-4-methoxy-2-((3-methyl-4-(1-methylpiperidin-4-yl)phenyl)amino)pyrimidine-5-carboxamide (Compound 134)

To a stirred solution of N-(2-bromo-6-cyanophenyl)-2-chloro-4-methoxy pyrimidine-5-carboxamide (75 mg, 0.204 mmol) was added 3-methyl-4-(1-methylpiperidin-4-yl)aniline (46 mg, 0.224 mmol) and the reaction mixture was stirred at 75° C. for 1 h under microwave irradiation to afford N-(2-bromo-6-cyanophenyl)-4-methoxy-2-((3-methyl-4-(1-methylpiperidin-4-yl)phenyl) amino)pyrimidine-5-carboxamide (35 mg, 32% yield) as an off-white solid. ¹H-NMR (400 MHz, DMSO-d₆): δ 10.09 (s, 1H), 9.85 (s, 1H), 8.77 (s, 1H), 8.10 (dd, J=1.60, 8.20 Hz, 1H), 7.96 (dd, J=1.60, 7.80 Hz, 1H), 7.58-7.55 (m, 2H), 7.46 (t, J=8.00 Hz, 1H), 7.17 (d, J=8.40 Hz, 1H), 4.14 (s, 3H), 2.90-2.85 (m, 2H), 2.63-2.54 (m, 1H), 2.30 (s, 3H), 2.20 (s, 3H), 2.02-1.92 (m, 2H), 1.68-1.60 (m, 4H). Chemical Formula: C₂₆H₂₇BrN₆O₂, Exact Mass: 534.1, Molecular Weight: 535.4. LCMS (ESI) m/z=535.3/537.2/538.2 (M+H), bromine isotopes, t_{R}=1.471 min, 99.3% (Method-A), HPLC purity: 98.9%; t_{R}=5.441 min (Method-B).

### Example 31. Synthesis of N-(2,6-dichlorophenyl)-4-methoxy-2-((2,4,4-trimethyl-1,2,3,4-tetrahydro isoquinolin-7-yl)amino)pyrimidine-5-carboxamide (Compound 135)

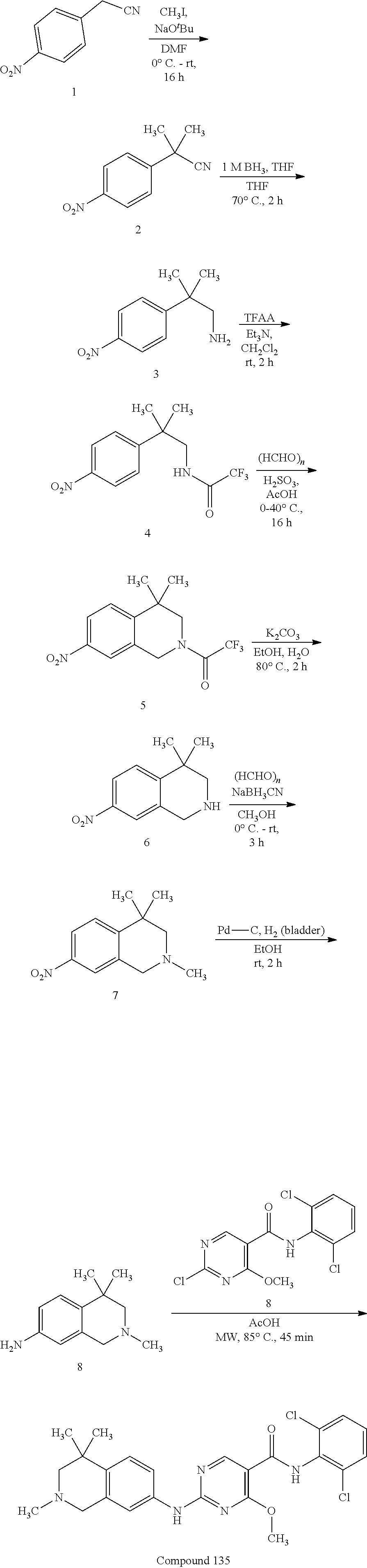

### 2-methyl-2-(4-nitrophenyl)propanenitrile

To a stirred solution of sodium tert-butoxide (17.78 g, 185 mmol) in DMF (100 mL) was added 2-(4-nitrophenyl)acetonitrile (15 g, 93 mmol) at 0° C. and stirred for 15 minutes. Then methyl iodide (26.3 g, 185 mmol) was added at 0° C. and stirred at room temperature for 15 min. To the resulting reaction mixture sodium tert-butoxide (17.78 g, 185 mmol) was added again at 0° C. and stirred for 15 minutes followed by the addition of methyl iodide (26.3 g, 185 mmol) at 0° C. The resulting reaction mixture was stirred at room temperature for 16 h. After completion of the reaction (TLC and UPLC), the reaction mixture was diluted with water and extracted with ethyl acetate (30 mL×2). The combined organic layer was dried over Na₂SO₄, filtered and concentrated under reduced pressure. The resulting crude material was purified by flash chromatography (SiO₂/100-200 mesh; 0-100% EtOAc/hexane) to afford 2-methyl-2-(4-nitrophenyl)propanenitrile (9 g, 51% yield) as an off white solid ¹H-NMR (400 MHz, DMSO-d₆): δ 8.29 (d, J=8.80 Hz, 2H), 7.70 (d, J=8.80 Hz, 2H), 1.81 (s, 6H). Chemical Formula: C₁₀H₁₀N₂O₂, Exact Mass: 190.1, Molecular Weight: 190.2. Note: LCMS: Product did not ionize.

### 2-methyl-2-(4-nitrophenyl)propan-1-amine

To a stirred solution of 2-methyl-2-(4-nitrophenyl)propanenitrile (18 g, 95 mmol) in THF (100 mL) was added 1M BH₃·THF (379 mL, 379 mmol) at 0° C. The reaction mixture was stirred at 70° C. for 2 h. After completion of the reaction (TLC and UPLC), the reaction mixture was quenched with methanol (400 mL) and concentrated under reduced pressure. The resultant crude material was dissolved in ethyl acetate and washed with 1.5 N HCl (250 mL). The separated aqueous layer was basified with aqueous 10% NaOH solution and extracted with ethyl acetate (200 mL×2). The combined organic layer was dried over Na₂SO₄, filtered and concentrated under reduced pressure to afford 2-methyl-2-(4-nitrophenyl)propan-1-amine (9 g, 48% yield) as a yellow solid. ¹H-NMR (400 MHz, DMSO-d₆): δ 8.21-8.19 (m, 2H), 7.54-7.52 (m, 2H), 2.88 (s, 2H), 1.37 (s, 6H), 1.33-1.25 (m, 2H). Chemical Formula: C₁₀H₁₄N₂O₂, Exact Mass: 194.1, Molecular Weight: 194.2. LCMS (ESI) im/z=195.0 (M+H), t_{R}=1.376 min, 99.4% (Method-B).

### 2,2,2-trifluoro-N-(2-methyl-2-(4-nitrophenyl)propyl)acetamide

To a stirred solution of 2-methyl-2-(4-nitrophenyl)propan-1-amine (9 g, 46.3 mmol) in CH₂Cl₂ (100 mL) was added triethylamine (4.69 g, 46.3 mmol) and TFAA (9.73 g, 46.3 mmol) at 0° C. under nitrogen atmosphere. The reaction mixture was stirred at room temperature for 2 h. After completion of the reaction (TLC and UPLC), the reaction mixture was diluted with water and extracted with CH₂Cl₂ (100 mL×2). The combined organic layer was dried over Na₂SO₄, filtered and concentrated under reduced pressure. The resulting crude material was purified by flash chromatography (SiO₂/100-200 mesh; 0-100% Ethyl acetate/Hexane) to afford 2,2,2-trifluoro-N-(2-methyl-2-(4-nitrophenyl)propyl)acetamide (9 g, 60% yield) as a yellow oil. ¹H-NMR (400 MHz, DMSO-d₆): δ 8.24-8.21 (m, 2H), 7.58-7.54 (m, 2H), 6.13 (s, 1H), 3.62 (d, J=6.40 Hz, 2H), 1.45 (s, 6H). Chemical Formula: C₁₂H₁₃F₃N₂O₃, Exact Mass: 290.1, Molecular Weight: 290.2. Note: LCMS: Product did not ionize.

### 1-(4,4-dimethyl-7-nitro-3,4-dihydroisoquinolin-2(1H)-yl)-2,2,2-trifluoroethan-1-one

To a stirred solution of 2,2,2-trifluoro-N-(2-methyl-2-(4-nitrophenyl)propyl) acetamide (9 g, 31.0 mmol) in acetic acid (90 mL) was added paraformaldehyde (1.5 g, 50.0 mmol) and H₂SO₄ (67.8 mL, 1265 mmol) at 0° C. and stirred at 40° C. for 16 h. After completion of the reaction (TLC and UPLC), the reaction mixture was concentrated under reduced pressure. The resulting crude material was dissolved in 10% aqueous NaHCO₃ (150 mL) solution and extracted with ethyl acetate (100 mL×2). The combined organic layer was dried over Na₂SO₄, filtered and concentrated under reduced pressure. The resulting crude material was purified by flash chromatography (SiO₂/100-200 mesh; 0-100% ethyl acetate/Hexane) to afford 1-(4,4-dimethyl-7-nitro-3,4-dihydroisoquinolin-2(1H)-yl)-2,2,2-trifluoroethan-1-one (5 g, 51% yield) as an off-white solid. ¹H-NMR (400 MHz, DMSO-d₆): δ 8.29-8.22 (m, 1H), 8.11-8.08 (m, 1H), 7.77-7.74 (m, 1H), 4.99-4.92 (m, 2H), 3.72-3.63 (m, 2H), 1.31-1.28 (m, 6H). Chemical Formula: C₁₃H₁₃F₃N₂O₃, Exact Mass: 302.1, Molecular Weight: 302.3. LCMS (ESI) m/z=302.1 (M), t_{R}=2.759 min, 95.7% (Method-A).

### 4,4-dimethyl-7-nitro-1,2,3,4-tetrahydroisoquinoline

To a stirred solution of 1-(4,4-dimethyl-7-nitro-3,4-dihydroisoquinolin-2(1H)-yl)-2,2,2-trifluoroethan-1-one (8.5 g, 28.1 mmol) in ethanol (50 mL) and water (10 mL), was added K₂CO₃ (15.55 g, 112 mmol) at room temperature. The resulting reaction mixture was stirred at 80° C. for 1 h. After completion of the reaction (TLC and UPLC), the reaction mixture was diluted with water and extracted with ethyl acetate (50 mL×2). The combined organic layer was dried over Na₂SO₄, filtered and concentrated under reduced pressure to afford as 4,4-dimethyl-7-nitro-1,2,3,4-tetrahydroisoquinoline (7.9 g, crude) as a yellow sticky solid that was taken into the next step without further purification. ¹H-NMR (400 MHz, CDCl₃): δ 8.05-8.02 (m, 1H), 7.90-7.89 (m, 1H), 7.50 (d, J=8.40 Hz, 1H), 4.12 (s, 2H), 2.92 (s, 2H), 1.38-1.34 (m, 7H). Chemical Formula: C₁₁H₁₄N₂O₂, Exact Mass: 206.1, Molecular Weight: 206.2. LCMS (ESI) m/z=207.2 (M+H), t_{R}=0.878 min, 96.4% (Method-A).

### 2,4,4-trimethyl-7-nitro-1,2,3,4-tetrahydroisoquinoline

To a stirred solution of 4,4-dimethyl-7-nitro-1,2,3,4-tetrahydroisoquinoline (2.5 g, 12.12 mmol) in methanol (60 mL), were added formaldehyde (1.4 mL, 18.18 mmol), AcOH (2 drops) at 0° C. The resulting reaction mixture was stirred at room temperature for 30 minutes. After this time sodium cyanoborohydride (1.143 g, 18.18 mmol) was added to the reaction mixture at room temperature stirred for 3 h. After completion of the reaction (TLC and UPLC), the reaction mixture was diluted with water and extracted with CH₂Cl₂ (50 mL×2). The combined organic layer was dried over Na₂SO₄, filtered and concentrated under reduced pressure. The resulting crude material was purified by flash chromatography (SiO₂/100-200 mesh; 0-100% ethyl acetate/Hexane) to afford 2,4,4-trimethyl-7-nitro-1,2,3,4-tetrahydroisoquinoline (2.1 g, 79% yield) as a yellow solid. ¹H-NMR (400 MHz, DMSO-d₆): δ 8.03-8.00 (m, 1H), 7.95-7.94 (m, 1H), 7.66 (d, J=8.80 Hz, 1H), 3.58 (s, 2H), 2.39-2.35 (m, 5H), 1.29 (s, 6H). Chemical Formula: C₁₂H₁₆N₂O₂, Exact Mass: 220.1, Molecular Weight: 220.3. Note: LCMS: Product did not ionize.

### 2,4,4-trimethyl-1,2,3,4-tetrahydroisoquinolin-7-amine

To a stirred solution of 2,4,4-trimethyl-7-nitro-1,2,3,4-tetrahydroisoquinoline (2.3 g, 10.44 mmol) in ethanol (120 mL), was added 10% Pd—C(1.15 g, 50% w/w) at room temperature under nitrogen atmosphere. The resulting reaction mixture was stirred at room temperature for 2 h under H₂ (bladder) atmosphere. After completion of the reaction (TLC and UPLC), the reaction mixture was filtered through celite, and filtrate was concentrated under reduced pressure. The resulting crude material was purified by reverse phase chromatography (C18 column; 0-100% CH₃CN in water) to afford 2,4,4-trimethyl-1,2,3,4-tetrahydroisoquinolin-7-amine (1.4 g, 71% yield) as a yellow solid. ¹H-NMR (400 MHz, DMSO-d₆): δ 6.96 (d, J=8.40 Hz, 1H), 6.41-6.38 (m, 1H), 6.17 (d, J=2.40 Hz, 1H), 4.76 (s, 2H), 3.28 (s, 2H), 2.27-2.24 (m, 5H), 1.16 (s, 6H). Chemical Formula: C₁₂H₁₈N₂, Exact Mass: 190.1, Molecular Weight: 190.3. LCMS (ESI) m/z=191.2 (M+H), t_{R}=1.419 min, 99.3% (Method-B), HPLC purity: 99.8%; t_{R}=4.200 min.

### N-(2,6-dichlorophenyl)-4-methoxy-2-((2,4,4-trimethyl-1,2,3,4-tetrahydro isoquinolin-7-yl)amino)pyrimidine-5-carboxamide (Compound 135)

To a stirred solution of 2,4,4-trimethyl-1,2,3,4-tetrahydroisoquinolin-7-amine (75 mg, 0.394 mmol) in acetic acid (1 mL), was added a 2-chloro-N-(2,6-dichlorophenyl)-4-methoxypyrimidine-5-carboxamide (105 mg, 0.315 mmol) at room temperature. The resulting reaction mixture was stirred at 85° C. for 45 minutes under microwave irradiation. After completion of the reaction (TLC and UPLC), the reaction mixture was concentrated under reduced pressure. The resulting crude was dissolved in 10% aqueous NaHCO₃ solution and extracted with EtOAc (10 mL×2). The resultant crude material was purified by prep. HPLC to get N-(2,6-dichlorophenyl)-4-methoxy-2-((2,4,4-trimethyl-1,2,3,4-tetrahydroisoquinolin-7-yl)amino)pyrimidine-5-carboxamide; formic acid salt (Compound 135, 33 mg, 17% yield) as an off-white solid. ¹H-NMR (400 MHz, DMSO-d₆): δ 10.05 (s, 1H), 9.56 (s, 1H), 8.74 (s, 1H), 8.25 (s, 1H), 7.58 (d, J=8.00 Hz, 2H), 7.54-7.49 (m, 2H), 7.41-7.37 (m, 1H), 7.31-7.29 (m, 1H), 4.11 (s, 3H), 3.45 (s, 2H), 2.34-2.33 (m, 5H), 1.24 (s, 6H). Note: one proton is extra for HCOOH salt. Chemical Formula: C₂₄H₂₅Cl₂N₅O₂, Exact Mass: 485.1, Molecular Weight: 485.4. LCMS (ESI) m/z=486.2/488.2/490.2 (M+H) chlorine isotopes, t_{R}=1.479 min, 99.8% (Method-A), HPLC purity: 98.8%; t_{R}=3.839 min. (Method-A).

### Example 32. Synthesis of N-(2-chloro-5-hydroxyphenyl)-4-methoxy-2-((3-methyl-4-(1-methylpiperidin-4-yl)phenyl)amino)pyrimidine-5-carboxamide (Compound 138)

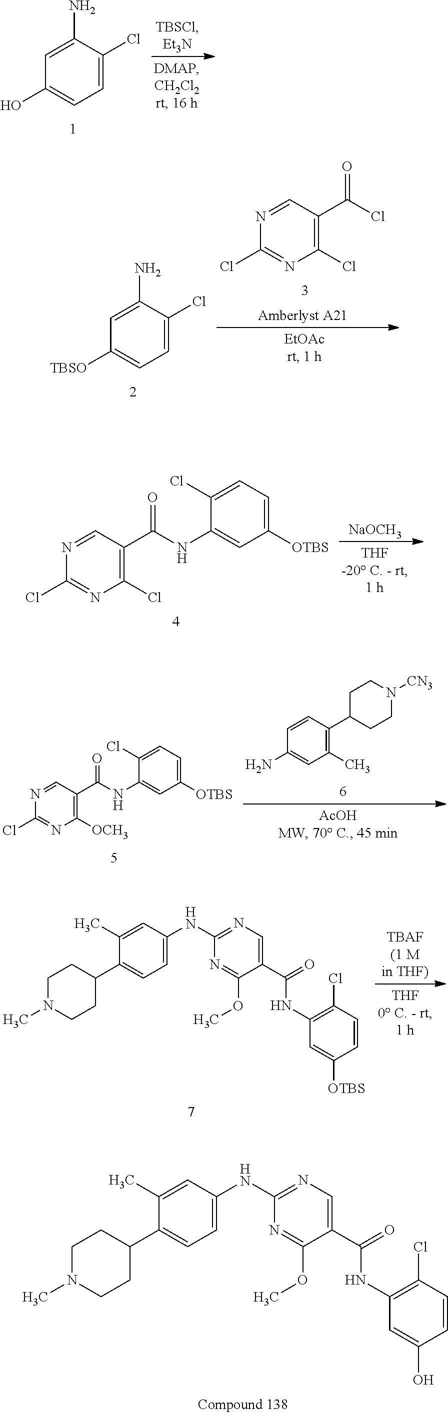

### 5-((tert-butyldimethylsilyl)oxy)-2-chloroaniline

To a stirred solution of 3-amino-4-chlorophenol (200 mg, 1.393 mmol) was added TBS Chloride (630 mg, 4.18 mmol) and the reaction mixture was stirred at room temperature for 16 h to afford 5-((tert-butyldimethylsilyl)oxy)-2-chloroaniline (320 mg, 89% yield) as a yellow liquid. ¹H-NMR (400 MHz, DMSO-d₆): δ 7.01 (d, J=8.80 Hz, 1H), 6.33 (d, J=2.80 Hz, 1H), 6.04 (dd, J=2.80, 8.80 Hz, 1H), 5.27 (s, 2H), 0.94 (s, 9H), 0.17 (s, 6H). Chemical Formula: C₁₂H₂₀ClNOSi, Exact Mass: 257.1, Molecular Weight: 257.8. LCMS (ESI) m/z=258.2/260.1/261.2 (M+H), Chlorine isotopes, t_{R}=2.580 min, 99.3% (Method-A).

### N-(5-((tert-butyldimethylsilyl)oxy)-2-chlorophenyl)-2,4-dichloropyrimidine-5-carboxamide

To a stirred solution of 2,4-dichloropyrimidine-5-carbonyl chloride (600 mg, 2.84 mmol) was added 5-((tert-butyldimethylsilyl)oxy)-2-chloroaniline (732 mg, 2.84 mmol) and the reaction mixture was stirred at room temperature for 1 h to afford N-(5-((tert-butyldimethylsilyl)oxy)-2-chlorophenyl)-2,4-dichloropyrimidine-5-carboxamide (500 mg, 33% yield) as an off white solid. Chemical Formula: Cl₇H₂OCl₃N₃O₂Si, Exact Mass: 431.0, Molecular Weight: 432.8. LCMS (ESI) m/z=434.0 (M+H), t_{R}=2.711 min, 81.5% (Method-A).

### N-(5-((tert-butyldimethylsilyl)oxy)-2-chlorophenyl)-2-chloro-4-methoxy pyrimidine-5-carboxamide

To a stirred solution of N-(5-((tert-butyldimethyl silyl)oxy)-2-chlorophenyl)-2,4-dichloropyrimidine-5-carboxamide (280 mg, 0.647 mmol) was added sodium methanolate (52.4 mg, 0.970 mmol) and the reaction mixture was stirred at room temperature for 1 h to afford N-(5-((tert-butyldimethylsilyl)oxy)-2-chlorophenyl)-2-chloro-4-methoxy pyrimidine-5-carboxamide (180 mg, 65% yield) as an off white solid. Chemical Formula: C₁₈H₂₃Cl₂N₃O₃Si, Exact Mass: 427.1, Molecular Weight: 428.4. LCMS (ESI) m/z=428.3 (M+H), t_{R}=2.881 min, 89.9% (Method-A).

### N-(5-((tert-butyldimethylsilyl)oxy)-2-chlorophenyl)-4-methoxy-2-((3-methyl-4-(1-methylpiperidin-4-yl)phenyl)amino)pyrimidine-5-carboxamide

To a stirred solution of N-(5-((tert-butyldimethylsilyl)oxy)-2-chlorophenyl)-2-chloro-4-methoxypyrimidine-5-carboxamide (200 mg, 0.467 mmol) was added 3-methyl-4-(1-methylpiperidin-4-yl)aniline (114 mg, 0.560 mmol) and the reaction mixture was stirred at 70° C. for 45 minutes under microwave irradiation to afford N-(5-((tert-butyldimethylsilyl)oxy)-2-chlorophenyl)-4-methoxy-2-((3-methyl-4-(1-methyl piperidin-4-yl)phenyl)amino) pyrimidine-5-carboxamide (200 mg, 58% yield) as an off-white solid. Chemical Formula: C₃₁H₄₂ClN₅O₃Si, Exact Mass: 595.3, Molecular Weight: 596.2. LCMS (ESI) m/z=596.3/598.2/599.3 (M+H), Chlorine isotopes, t_{R}=2.201 min, 81.9% (Method-A).

### N-(2-chloro-5-hydroxyphenyl)-4-methoxy-2-((3-methyl-4-(I-methyl piperidin-4-yl)phenyl)amino)pyrimidine-5-carboxamide (Compound 138)

To a stirred solution of N-(5-((tert-butyldimethylsilyl)oxy)-2-chlorophenyl)-4-methoxy-2-((3-methyl-4-(1-methylpiperidin-4-yl)phenyl)amino)pyrimidine-5-carboxamide (200 mg, 0.335 mmol) was added TBAF (0.503 ml, 0.503 mmol) and the reaction mixture was stirred at room temperature for 1 h under nitrogen atmosphere to afford N-(2-chloro-5-hydroxyphenyl)-4-methoxy-2-((3-methyl-4-(1-methylpiperidin-4-yl)phenyl)amino) pyrimidine-5-carboxamide (25 mg, 15% yield) as an off-white solid. ¹H-NMR (400 MHz, DMSO-d₆): δ 10.14 (s, 1H), 9.89 (s, 1H), 9.82 (s, 1H), 8.87 (s, 1H), 8.07 (d, J=2.80 Hz, 1H), 7.56 (d, J=6.00 Hz, 2H), 7.31 (d, J=8.80 Hz, 1H), 7.18 (d, J=8.80 Hz, 1H), 6.55 (dd, J=2.80, 8.60 Hz, 1H), 4.20 (s, 3H), 2.89-2.86 (m, 2H), 2.30 (s, 3H), 2.20 (s, 3H), 2.01-1.95 (m, 2H), 1.69-1.60 (m, 4H). Note: One proton merged in NMR solvent peak. Chemical Formula: C₂₅H₂₈C₁N₅03, Exact Mass: 481.2, Molecular Weight: 482.0. LCMS (ESI) m/z=482.2/483.2 (M+H), Chlorine isotopes, t_{R}=1.558 min, 97.8% (Method-A), HPLC purity: 98.7%; t_{R}=3.940 min (Method-A).

### Example 33. Synthesis of N-(6-chloro-3-hydroxy-2-methylphenyl)-4-methoxy-2-((3-methyl-4-(1-methyl piperidin-4-yl)phenyl)amino)pyrimidine-5-carboxamide (Compound 141)

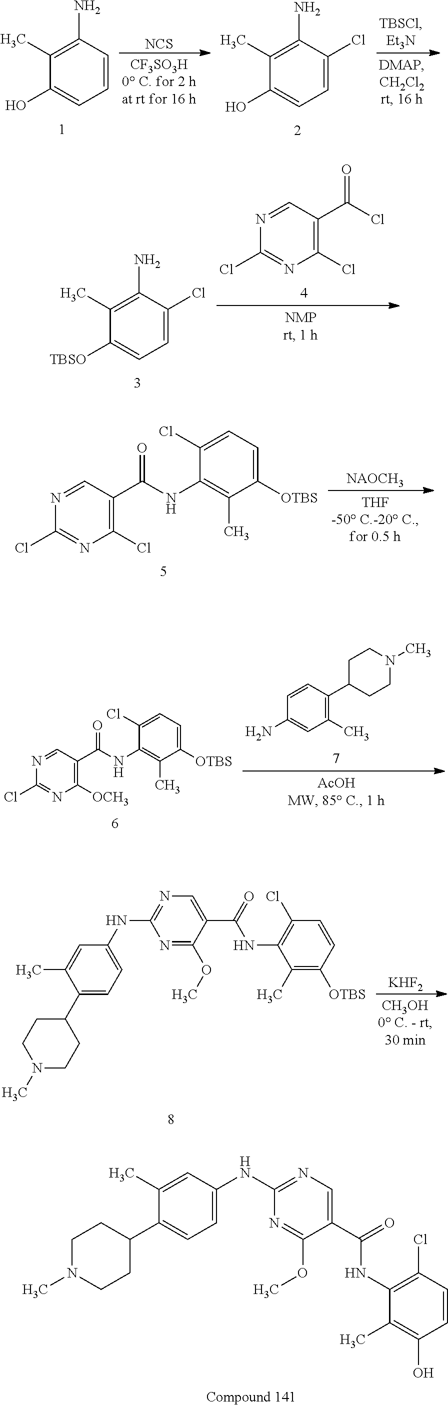

### 3-amino-4-chloro-2-methylphenol

To a stirred solution of 3-amino-2-methylphenol (3.0 g, 24.36 mmol) in methane sulphonic acid (30 mL) was added N-chlorosuccinimide (3.58 g, 26.8 mmol) at 0° C. The reaction mixture was stirred at room temperature for 16 h. After completion of the reaction (TLC and UPLC), the reaction mixture was neutralized with concentrated ammonia solution and extracted with ethyl acetate (100 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 3-amino-4-chloro-2-methylphenol (2.23 g, 57% yield) as an off-white solid. ¹H-NMR (400 MHz, DMSO-d₆): δ 9.11 (s, 1H), 6.85-6.80 (m, 1H), 6.19-6.12 (m, 1H), 4.83 (s, 2H), 1.95 (d, J=2.40 Hz, 3H). Chemical Formula: C₇H₈ClNO, Exact Mass: 157.0, Molecular Weight: 157.6. LCMS (ESI) m/z=158.1 (M+H), t_{R}=1.339 min, 73.5% (Method-B). Note: Regioselectivity was confirmed by 2D nOe.

### 3-((tert-butyldimethylsilyl)oxy)-6-chloro-2-methylaniline

To a stirred solution of 3-amino-4-chloro-2-methylphenol (1 g, 6.35 mmol) was added TBS Chloride (2.87 g, 19.04 mmol) and the reaction mixture was stirred at room temperature for 16 h to afford 3-((tert-butyldimethylsilyl)oxy)-6-chloro-2-methylaniline (480 mg, 28% yield) as a colourless liquid. ¹H-NMR (400 MHz, DMSO-d₆): δ 6.94 (d, J=8.80 Hz, 1H), 6.13 (d, J=8.40 Hz, 1H), 4.97 (s, 2H), 1.99 (s, 3H), 0.98 (s, 9H), 0.17 (s, 6H). Chemical Formula: Cl₃H₂₂ClNOSi, Exact Mass: 271.1, Molecular Weight: 271.9. LCMS (ESI) m/z=272.2/273.2 (M+H), Chlorine isotopes, t_{R}=3.282 min, 99.2% (Method-A).

### N-(3-((tert-butyldimethylsilyl)oxy)-6-chloro-2-methylphenyl)-2,4-dichloropyrimidine-5-carboxamide

To a stirred solution of 3-((tert-butyldimethylsilyl)oxy)-6-chloro-2-methylaniline (400 mg, 1.471 mmol) was added 2,4-dichloropyrimidine-5-carbonyl chloride (373 mg, 1.766 mmol) and the reaction mixture was stirred at room temperature for 1 h to afford N-(3-((tert-butyldimethylsilyl)oxy)-6-chloro-2-methylphenyl)-2,4-dichloropyrimidine-5-carboxamide (330 mg, 48% yield) as a colourless gum. Chemical Formula: C₁₈H₂₂Cl₃N₃O₂Si, Exact Mass: 445.1, Molecular Weight: 446.8. LCMS (ESI) m/z=448.0 (M+H), t_{R}=2.437 min, 95.5% (Method-B).

### N-(3-((tert-butyldimethylsilyl)oxy)-6-chloro-2-methylphenyl)-2-chloro-4-methoxypyrimidine-5-carboxamide

To a stirred solution of N-(3-((tert-butyldimethylsilyl)oxy)-6-chloro-2-methylphenyl)-2,4-dichloropyrimidine-5-carboxamide (220 mg, 0.492 mmol) in THF (5 mL) was added sodium methanolate (39.9 mg, 0.739 mmol) at −50° C. The reaction mixture was stirred at −20° C. for 30 minutes. After completion of the reaction (TLC and UPLC), the reaction mixture was diluted with water and extracted with EtOAc (20 mL×2). The combined organic layer was dried over Na₂SO₄, filtered and concentrated under reduced pressure to afford N-(3-((tert-butyldimethylsilyl)oxy)-6-chloro-2-methylphenyl)-2-chloro-4-methoxypyrimidine-5-carboxamide (200 mg, 81% yield) as a colourless gum. Chemical Formula: C₁₉H₂₅Cl₂N₃O₃Si, Exact Mass: 441.1, Molecular Weight: 442.4. LCMS (ESI) m/z=442.3 (M+H), t_{R}=2.581 min, 88.8% (Method-B).

### N-(3-((tert-butyldimethylsilyl)oxy)-6-chloro-2-methylphenyl)-4-methoxy-2-((3-methyl-4-(1-methylpiperidin-4-yl)phenyl)amino)pyrimidine-5-carboxamide

To a stirred solution of N-(3-((tert-butyldimethylsilyl)oxy)-6-chloro-2-methylphenyl)-2-chloro-4-methoxypyrimidine-5-carboxamide (200 mg, 0.452 mmol) was added 3-methyl-4-(1-methylpiperidin-4-yl)aniline (92 mg, 0.452 mmol) and the reaction mixture was stirred at 85° C. for 1 h under microwave irradiation to afford N-(3-((tert-butyldimethylsilyl)oxy)-6-chloro-2-methylphenyl)-4-methoxy-2-((3-methyl-4-(1-methylpiperidin-4-yl)phenyl)amino)pyrimidine-5-carboxamide (200 mg, 23% yield) as a yellow white solid. Chemical Formula: C₃₂H₄₄ClN₅O₃Si, Exact Mass: 609.3, Molecular Weight: 610.3. LCMS (ESI) m/z=610.4/611.4, Chlorine isotopes (M+H), t_{R}=2.610 min, 32.1% (Method-A).

### N-(6-chloro-3-hydroxy-2-methylphenyl)-4-methoxy-2-((3-methyl-4-(1-methylpiperidin-4-yl)phenyl)amino)pyrimidine-5-carboxamide (Compound 141

To a stirred solution of N-(3-((tert-butyldimethylsilyl)oxy)-6-chloro-2-methylphenyl)-4-methoxy-2-((3-methyl-4-(1-methylpiperidin-4-yl)phenyl) amino) pyrimidine-5-carboxamide (200 mg, 0.328 mmol) was added potassium hydrogen fluoride (38.4 mg, 0.492 mmol). The resultant crude residue was purified by prep. HPLC to get N-(6-chloro-3-hydroxy-2-methylphenyl)-4-methoxy-2-((3-methyl-4-(1-methyl piperidin-4-yl)phenyl)amino)pyrimidine-5-carboxamide (15 mg, 9% yield) as an off-white solid. ¹H-NMR (400 MHz, DMSO-d₆): δ 9.97 (s, 1H), 9.29 (s, 1H), 8.72 (s, 1H), 8.23 (s, 1H), 7.58-7.55 (m, 2H), 7.18-7.15 (m, 2H), 6.80 (d, J=8.80 Hz, 1H), 4.11 (s, 3H), 2.93-2.91 (m, 2H), 2.66-2.56 (m, 1H), 2.29 (s, 3H), 2.24 (s, 3H), 2.10-2.03 (m, 5H), 1.66-1.65 (m, 4H). Chemical Formula: C₂₆H₃₀C₁N₅O₃, Exact Mass: 495.2, Molecular Weight: 496.0. LCMS (ESI) m/z=496.2/498.2, Chlorine isotopes (M+H), t_{R}=1.850 min, 99.8% (Method-A), HPLC purity: 99.5%; t_{R}=3.509 min (Method-A).

### Example 34. Synthesis of 2-((3-cyano-4-(1-methylpiperidin-4-yl)phenyl)amino)-N-(2,6-dichlorophenyl)-4-methoxypyrimidine-5-carboxamide (Compound 156)

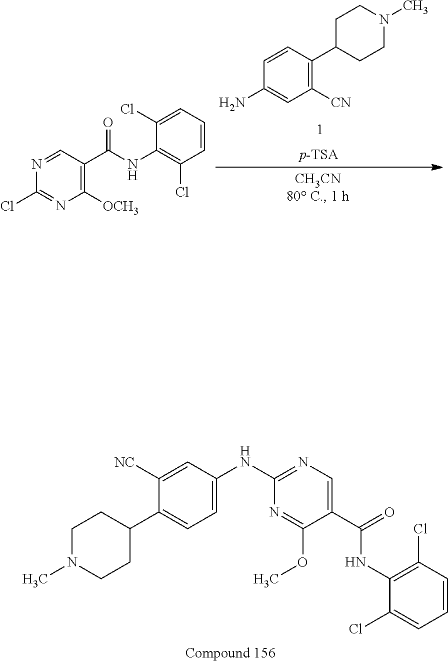

To a stirred solution of 2-chloro-N-(2,6-dichlorophenyl)-4-methoxypyrimidine-5-carboxamide (100 mg, 0.301 mmol) in acetonitrile (1 mL) was added 5-amino-2-(1-methylpiperidin-4-yl)benzonitrile (64.7 mg, 0.301 mmol) and p-TSA (64.7 mg, 0.301 mmol) at room temperature and was stirred at 80° C. for 1 h. After completion of the reaction (TLC and UPLC), the reaction mixture was concentrated under reduced pressure. The resulting crude material was purified by Prep. HPLC to afford 2-((3-cyano-4-(1-methylpiperidin-4-yl)phenyl)amino)-N-(2,6-dichlorophenyl)-4-methoxypyrimidine-5-carboxamide (Compound 156, 22 mg, 14% yield) as an off-white solid. ¹H-NMR (400 MHz, DMSO-d₆): δ 10.40 (s, 1H), 9.64 (s, 1H), 8.80 (s, 1H), 8.24 (d, J=2.40 Hz, 1H), 7.99 (dd, J=2.40, 8.80 Hz, 1H), 7.59 (d, J=8.00 Hz, 2H), 7.51 (d, J=8.80 Hz, 1H), 7.41-7.37 (m, 1H), 4.13 (s, 3H), 2.94-2.89 (m, 2H), 2.79-2.70 (m, 1H), 2.21 (s, 3H), 2.02-1.95 (m, 2H), 1.79-1.70 (m, 4H). Chemical Formula: C₂₅H₂₄Cl₂N₆O₂, Exact Mass: 510.1, Molecular Weight: 511.4. LCMS (ESI) m/z=511.2 (M+H), t_{R}=2.093 min, 99.2% (Method-B), HPLC purity: 99.6%; t_{R}=3.831 min (Method-A).

### Example 35. Synthesis of N-(6-chloro-3-hydroxy-2-methylphenyl)-2-((3-cyano-4-(1-methylpiperidin-4-yl)phenyl)amino)-4-methoxypyrimidine-5-carboxamide (Compound 157)

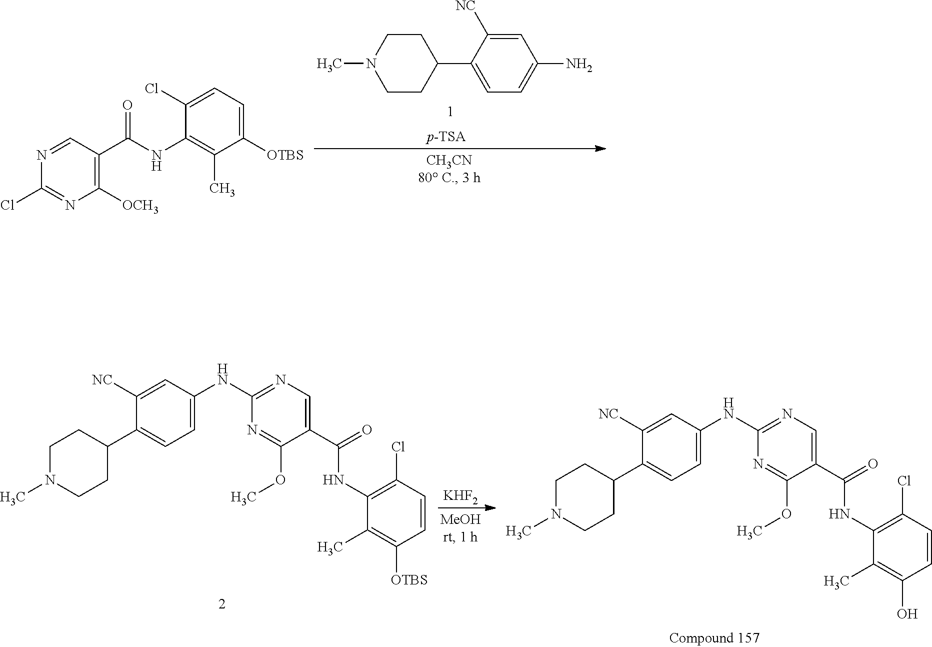

### N-(3-((tert-butyldimethylsilyl)oxy)-6-chloro-2-methylphenyl)-2-((3-cyano-4-(1-methylpiperidin-4-yl)phenyl)amino)-4-methoxypyrimidine-5-carboxamide

To a stirred solution of N-(3-((tert-butyldimethylsilyl)oxy)-6-chloro-2-methylphenyl)-2-chloro-4-methoxypyrimidine-5-carboxamide (100 mg, 0.226 mmol) in acetonitrile (2 mL) was added 5-amino-2-(1-methylpiperidin-4-yl)benzonitrile (58.4 mg, 0.271 mmol) and p-toluenesulfonic acid (86 mg, 0.452 mmol) at room temperature and was stirred at 80° C. for 3 h. After completion of the reaction (TLC and UPLC), the reaction mixture was diluted with water and extracted with ethyl acetate (20 mL×2). The combined organic layer was dried over Na₂SO₄, filtered and concentrated under reduced pressure to afford N-(3-((tert-butyldimethylsilyl)oxy)-6-chloro-2-methylphenyl)-2-((3-cyano-4-(1-methylpiperidin-4-yl)phenyl)amino)-4-methoxypyrimidine-5-carboxamide (130 mg, crude) as an off-white solid. This material was as such used for the next step without further purification. Chemical Formula: C₃₂H₄₁ClN₆O₃Si, Exact Mass: 620.3, Molecular Weight: 621.3. LCMS (ESI) im/z=621.5/622.5 (M+H), Chlorine isotopes, t_{R}=2.4 min, 30% (Method-C).

### N-(6-chloro-3-hydroxy-2-methylphenyl)-2-((3-cyano-4-(1-methylpiperidin-4-yl)phenyl)amino)-4-methoxypyrimidine-5-carboxamide (Compound 157)

To a stirred solution of N-(3-((tert-butyldimethylsilyl)oxy)-6-chloro-2-methylphenyl)-2-((3-cyano-4-(1-methylpiperidin-4-yl)phenyl)amino)-4-methoxypyrimidine-5-carboxamide (130 mg, 0.209 mmol) in methanol (2 mL), was added a potassium hydrogen fluoride (16.34 mg, 0.209 mmol) at 0° C. The resulting reaction mixture was stirred at room temperature for 1 h. After completion of the reaction (TLC and UPLC), the reaction mixture was concentrated under reduced pressure. The resulting crude material was purified by preparative HPLC to afford N-(6-chloro-3-hydroxy-2-methylphenyl)-2-((3-cyano-4-(1-methylpiperidin-4-yl)phenyl)amino)-4-methoxypyrimidine-5-carboxamide (Compound 157, 15 mg, 14% yield) as an off-white solid. ¹H-NMR (400 MHz, DMSO-d₆): δ 10.36 (s, 1H), 9.72 (s, 1H), 9.38 (s, 1H), 8.78 (s, 1H), 8.24 (d, J=2.40 Hz, 1H), 8.00 (dd, J=2.00, 8.60 Hz, 1H), 7.50 (d, J=8.80 Hz, 1H), 7.17 (d, J=8.40 Hz, 1H), 6.81 (d, J=8.80 Hz, 1H), 4.12 (s, 3H), 2.98-2.96 (m, 2H), 2.83-2.74 (m, 1H), 2.34 (s, 3H), 2.15-2.07 (m, 2H), 2.08 (s, 3H), 1.80-1.73 (m, 4H). Chemical Formula: C₂₆H₂₇ClN₆O₃, Exact Mass: 506.2, Molecular Weight: 507.0. LCMS (ESI) m/z=507.4/509.4 (M+H), Chlorine isotopes, t_{R}=1.37 min, 97% (Method-C), HPLC purity: 96.3%; t_{R}=5.963 min (Method-C).

### Example 36. Synthesis of N-(2,6-dichlorophenyl)-2-((4-(3-(dimethylamino)propoxy)-3-methylphenyl)amino)-4-methoxypyrimidine-5-carboxamide (Compound 147)

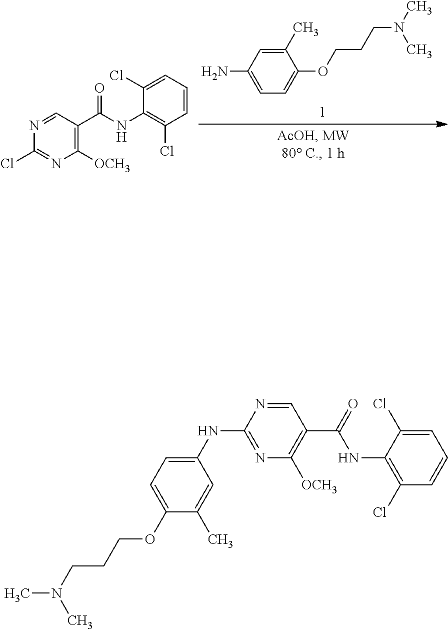

To a stirred solution of 2-chloro-N-(2,6-dichlorophenyl)-4-methoxypyrimidine-5-carboxamide (120 mg, 0.361 mmol) in acetic acid (2 mL) was added 4-(3-(dimethylamino)propoxy)-3-methylaniline (90 mg, 0.433 mmol) at room temperature and was stirred at 80° C. for 1 h under microwave irradiation. After completion of the reaction (TLC and UPLC), the reaction mixture was concentrated under reduced pressure. The resultant crude was basified with aqueous sodium bicarbonate and extracted with ethyl acetate (20 mL×2). The combined organic layer was dried over Na₂SO₄, filtered and concentrated under reduced pressure. The resulting crude material was purified by prep. HPLC to afford N-(2,6-dichlorophenyl)-2-((4-(3-(dimethylamino)propoxy)-3-methylphenyl)amino)-4-methoxypyrimidine-5-carboxamide (33 mg, 17% yield) as an off-white solid. ¹H-NMR (400 MHz, DMSO-d₆): δ 9.93 (s, 1H), 9.52 (s, 1H), 8.73 (s, 1H), 7.59-7.51 (m, 4H), 7.39 (t, J=8.00 Hz, 1H), 6.90 (d, J=8.80 Hz, 1H), 4.09 (s, 3H), 3.98 (t, J=6.40 Hz, 2H), 2.38 (t, J=7.20 Hz, 2H), 2.16-2.15 (m, 9H), 1.89-1.82 (m, 2H). Chemical Formula: C₂₄H₂₇Cl₂N₅O₃, Exact Mass: 503.1, Molecular Weight: 504.4. LCMS (ESI) m/z=504.3 (M+H), t_{R}=2.651 min, 96.5% (Method-B). HPLC purity: 97.0%; t_{R}=5.926 min (Method-B).

### Example 37. Synthesis of N-(2,6-dichlorophenyl)-2-((4-(2-(dimethylamino)ethoxy)-3-methylphenyl)amino)-4-methoxypyrimidine-5-carboxamide (Compound 143)

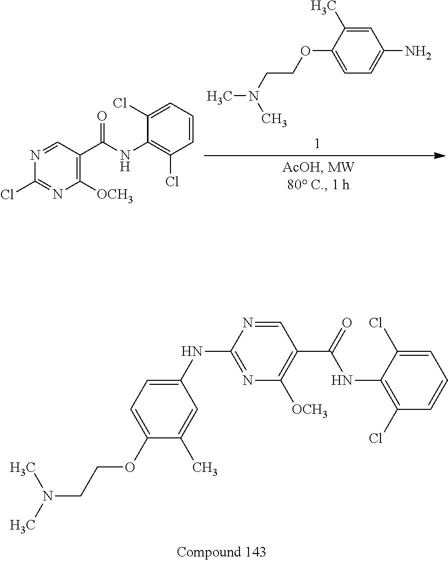

To a stirred solution of 2-chloro-N-(2,6-dichlorophenyl)-4-methoxypyrimidine-5-carboxamide (100 mg, 0.301 mmol) was added 4-(2-(dimethylamino)ethoxy)-3-methylaniline (70.1 mg, 0.361 mmol) s and the reaction mixture was stirred at 80° C. for 1 h under microwave irradiation to afford N-(2,6-dichlorophenyl)-2-((4-(2-(dimethylamino)ethoxy)-3-methylphenyl)amino)-4-methoxypyrimidine-5-carboxamide (Compound 143, 23 mg, 15% yield) as a pale yellow solid. ¹H-NMR (400 MHz, DMSO-d₆): δ 9.94 (s, 1H), 9.52 (s, 1H), 8.73 (s, 1H), 7.59-7.51 (m, 4H), 7.38 (t, J=8.40 Hz, 1H), 6.92 (d, J=8.80 Hz, 1H), 4.09 (s, 3H), 4.04 (t, J=5.60 Hz, 2H), 2.65 (t, J=5.60 Hz, 2H), 2.24 (s, 6H), 2.16 (s, 3H). Chemical Formula: C₂₃H₂₅Cl₂N₅O₃, Exact Mass: 489.1, Molecular Weight: 490.4. LCMS (ESI) m/z=490.3/492.3 (M+H), chlorine isotopes, t_{R}=1.6 min, 98.4% (Method-C), HPLC purity: 98.6%; t_{R}=3.783 min (Method-A).

### Example 38. Synthesis of N-(2,6-dichlorophenyl)-4-methoxy-2-((3-methyl-4-(3-morpholinopropoxy)phenyl)amino)pyrimidine-5-carboxamide (Compound 148)

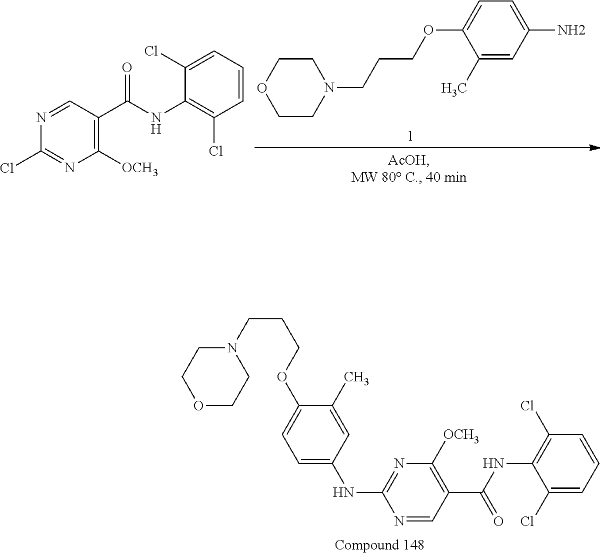

To a stirred solution of 2-chloro-N-(2,6-dichlorophenyl)-4-methoxypyrimidine-5-carboxamide (0.100 g, 0.301 mmol) was added 3-methyl-4-(3-morpholinopropoxy)aniline (0.083 g, 0.331 mmol) and the reaction mixture was stirred at 80° C. for 45 min under microwave irradiation to afford N-(2,6-dichlorophenyl)-4-methoxy-2-((3-methyl-4-(3-morpholinopropoxy)phenyl)amino)pyrimidine-5-carboxamide (Compound 148, 0.035 g, 21% yield) as an off white solid. ¹H-NMR (400 MHz, DMSO-d₆): δ 9.94 (s, 1H), 9.52 (s, 1H), 8.73 (s, 1H), 7.59-7.51 (m, 4H), 7.38 (t, J=8.00 Hz, 1H), 6.90 (d, J=8.80 Hz, 1H), 4.09 (s, 3H), 3.99 (t, J=6.40 Hz, 2H), 3.58 (t, J=4.80 Hz, 4H), 2.45 (t, J=7.20 Hz, 2H), 2.38-2.34 (m, 4H), 2.16 (s, 3H), 1.91-1.87 (m, 2H). Chemical Formula: C₂₆H₂₉Cl₂N₅O₄, Exact Mass: 545.2, Molecular Weight: 546.4. LCMS (ESI) m/z=546.4 (M+H), t_{R}=2.651 min, 98.1% (Method-C), HPLC purity: 99.4%; t_{R}=4.232 min (Method-A).

### Example 39. Synthesis of N-(2,6-dichlorophenyl)-4-methoxy-2-((3-methyl-4-(2-morpholinoethoxy)phenyl)amino)pyrimidine-5-carboxamide (Compound 144)

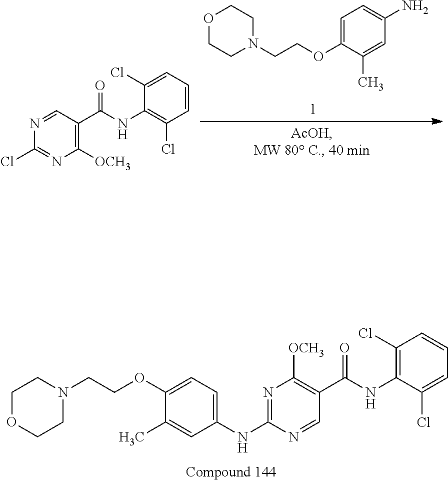 [SMILES: COc1nc(Cl)ncc1C(=O)Nc1c(Cl)cccc1Cl ; Cc1cc(N)ccc1OCCN1CCOCC1 ; COc1nc(Nc2ccc(OCCN3CCOCC3)c(C)c2)ncc1C(=O)Nc1c(Cl)cccc1Cl]

To a stirred solution of 2-chloro-N-(2,6-dichlorophenyl)-4-methoxypyrimidine-5-carboxamide (100 mg, 0.301 mmol) was added 3-methyl-4-(2-morpholinoethoxy)aniline (114 mg, 0.481 mmol) and the reaction mixture was stirred at 80° C. for 40 min under microwave irradiation to afford N-(2,6-dichlorophenyl)-4-methoxy-2-((3-methyl-4-(2-morpholinoethoxy)phenyl)amino)pyrimidine-5-carboxamide (Compound 144, 25 mg, 15% yield) as a pale brown solid. ¹H-NMR (400 MHz, CD₃OD): δ 8.84 (s, 1H), 7.53-7.47 (m, 4H), 7.34 (t, J=7.60 Hz, 1H), 6.94 (d, J=8.80 Hz, 1H), 4.22-4.19 (m, 5H), 3.77 (t, J=4.80 Hz, 4H), 2.98-2.92 (m, 2H), 2.79-2.72 (m, 4H), 2.26 (s, 3H). Note: Exchangeable protons not observed. Chemical Formula: C₂₅H₂₇Cl₂N₅O₄, Exact Mass: 531.1, Molecular Weight: 532.4. LCMS (ESI) m/z=532.6 (M+H), t_{R}=1.37 min, 95.7% (Method-A), HPLC purity: 96.7%; t_{R}=3.783 min (Method-A).

### Example 40. Synthesis of N-(2,6-dichlorophenyl)-4-methoxy-2-((3-methyl-4-((1-methylpiperidin-4-yl)oxy)phenyl)amino)pyrimidine-5-carboxamide (Compound 145)

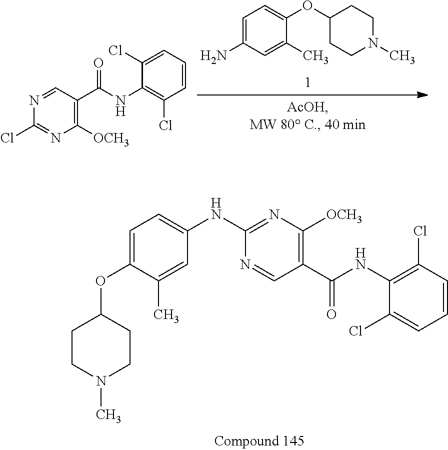

To a stirred solution of 2-chloro-N-(2,6-dichlorophenyl)-4-methoxypyrimidine-5-carboxamide (100 mg, 0.301 mmol) was added 3-methyl-4-((1-methylpiperidin-4-yl)oxy)aniline (72.9 mg, 0.331 mmol) and the reaction mixture was stirred at 80° C. for 40 min under microwave irradiation to afford N-(2,6-dichlorophenyl)-4-methoxy-2-((3-methyl-4-((1-methylpiperidin-4-yl)oxy)phenyl)amino)pyrimidine-5-carboxamide formic acid salt (Compound 145, 40 mg, 25% yield) as a pale brown solid. ¹H-NMR (400 MHz, DMSO-d₆): δ 9.94 (s, 1H), 9.52 (s, 1H), 8.72 (s, 1H), 8.16 (s, 1.5H), 7.62-7.50 (m, 4H), 7.38 (t, J=8.00 Hz, 1H), 6.95 (d, J=9.20 Hz, 1H), 4.37-4.32 (m, 1H), 4.09 (s, 3H), 2.70-2.67 (m, 2H), 2.47-2.36 (m, 2H), 2.30 (s, 3H), 2.18 (s, 3H), 1.96-1.92 (m, 2H), 1.74-1.71 (m, 2H). Chemical Formula: C₂₅H₂₇Cl₂N₅O₃, Exact Mass: 515.1, Molecular Weight: 516.4. LCMS (ESI) m/z=516.7 (M+H), t_{R}=1.34 min, 95.75% (Method-A), HPLC purity: 99.5%; t_{R}=3.918 min (Method-A).

### Example 41. Synthesis of (S)—N-(2,6-dichlorophenyl)-4-methoxy-2-((3-methyl-4-((1-methylpyrrolidin-3-yl)oxy)phenyl)amino)pyrimidine-5-carboxamide (Compound 154)

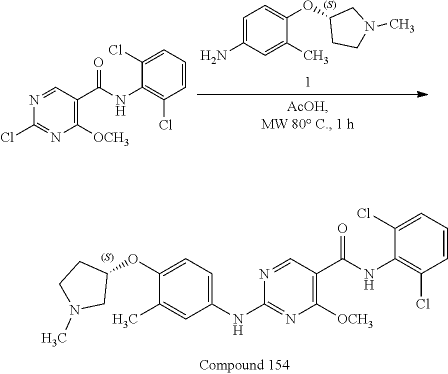

To a stirred solution of 2-chloro-N-(2,6-dichlorophenyl)-4-methoxypyrimidine-5-carboxamide (100 mg, 0.301 mmol) was added (S)-3-methyl-4-((1-methylpyrrolidin-3-yl)oxy)aniline (62.0 mg, 0.301 mmol) and the reaction mixture was stirred at 80° C. for 1 h under microwave irradiation to afford (S)—N-(2,6-dichlorophenyl)-4-methoxy-2-((3-methyl-4-((1-methylpyrrolidin-3-yl)oxy)phenyl)amino)pyrimidine-5-carboxamide (30 mg, 20% yield) as an off-white solid. ¹H-NMR (400 MHz, DMSO-d₆): δ 9.92 (s, 1H), 9.53 (d, J=6.00 Hz, 1H), 8.73 (d, J=6.40 Hz, 1H), 7.59-7.49 (m, 4H), 7.40-7.37 (m, 1H), 6.83 (d, J=8.80 Hz, 1H), 4.83-4.80 (m, 1H), 4.09 (s, 3H), 2.84-2.78 (m, 1H), 2.69-2.56 (m, 2H), 2.41-2.35 (m, 1H), 2.30-2.23 (m, 4H), 2.14 (s, 3H), 1.83-1.74 (m, 1H). Chemical Formula: C₂₄H₂₅Cl₂N₅O₃ Exact Mass: 501.1 Molecular Weight: 502.4. LCMS (ESI) m/z=502.2/504.2 (M+H), chlorine isotopes, t_{R}=1.891 min, 97.3% (Method-A), HPLC purity: 96.8%; t_{R}=3.855 min (Method-A).

### Example 42. Synthesis of (R)—N-(2,6-dichlorophenyl)-4-methoxy-2-((3-methyl-4-((1-methylpyrrolidin-3-yl)oxy)phenyl)amino)pyrimidine-5-carboxamide (Compound 146)

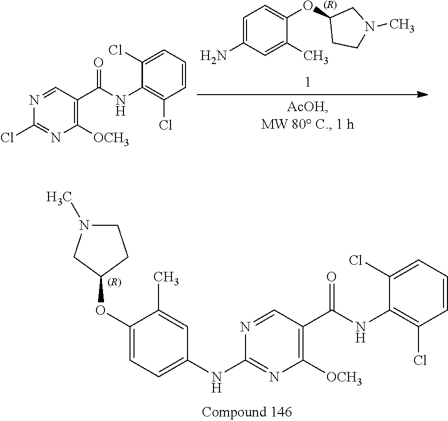

To a stirred solution of 2-chloro-N-(2,6-dichlorophenyl)-4-methoxypyrimidine-5-carboxamide (0.100 g, 0.301 mmol) was added (R)-3-methyl-4-((1-methylpyrrolidin-3-yl)oxy)aniline (0.068 g, 0.331 mmol) and the reaction mixture was stirred at 80° C. for 1 h under microwave irradiation to afford (R)—N-(2,6-dichlorophenyl)-4-methoxy-2-((3-methyl-4-((1-methylpyrrolidin-3-yl)oxy)phenyl)amino)pyrimidine-5-carboxamide (0.030 g, 19% yield) as a pale brown solid. ¹H-NMR (400 MHz, DMSO-d₆): δ 9.94 (s, 1H), 9.52 (s, 1H), 8.72 (s, 1H), 7.59-7.49 (m, 4H), 7.39 (t, J=8.00 Hz, 1H), 6.83 (d, J=8.80 Hz, 1H), 4.84-4.80 (m, 1H), 4.09 (s, 3H), 2.84-2.79 (m, 1H), 2.70-2.55 (m, 2H), 2.45-2.34 (m, 1H), 2.30-2.23 (m, 4H), 2.14 (s, 3H), 1.82-1.73 (m, 1H). Chemical Formula: C₂₄H₂₅Cl₂N₅O₃, Exact Mass: 501.1, Molecular Weight: 502.4. LCMS (ESI) m/z=502.3 (M+H), t_{R}=2.480 min, 98.4% (Method-C), HPLC purity: 98%; t_{R}=3.738 min (Method-A).

### Example 43. Synthesis of N-(6-chloro-3-hydroxy-2-methylphenyl)-2-((4-(3-(dimethylamino)propoxy)-3-methylphenyl)amino)-4-methoxypyrimidine-5-carboxamide (Compound 158)

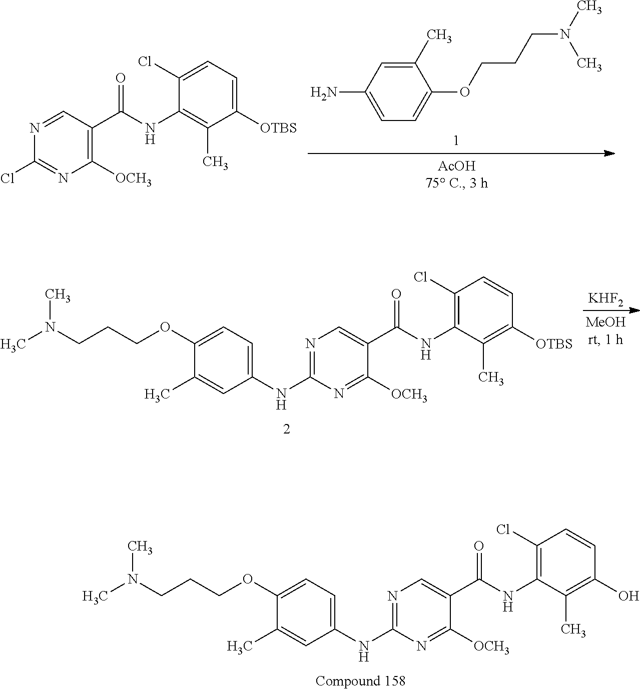 [SMILES: COc1nc(Cl)ncc1C(=O)Nc1c(Cl)ccc(O[Si](C)(C)C(C)(C)C)c1C ; Cc1cc(N)ccc1OCCCN(C)C ; COc1nc(Nc2ccc(OCCCN(C)C)c(C)c2)ncc1C(=O)Nc1c(Cl)ccc(O[Si](C)(C)C(C)(C)C)c1C ; "KHF2" ; COc1nc(Nc2ccc(OCCCN(C)C)c(C)c2)ncc1C(=O)Nc1c(Cl)ccc(O)c1C]

### N-(3-((tert-butyldimethylsilyl)oxy)-6-chloro-2-methylphenyl)-2-((4-(3-(dimethylamino)propoxy)-3-methylphenyl)amino)-4-methoxypyrimidine-5-carboxamide

To a stirred solution of N-(3-((tert-butyldimethylsilyl)oxy)-6-chloro-2-methylphenyl)-2-chloro-4-methoxypyrimidine-5-carboxamide (100 mg, 0.226 mmol) in acetic acid (2 mL) was added 4-(3-(dimethylamino)propoxy)-3-methylaniline (56.5 mg, 0.271 mmol) at room temperature and was stirred at 75° C. for 3 h. After completion of the reaction (TLC and UPLC), the reaction mixture was concentrated under reduced pressure. The resultant crude was basified with aqueous sodium bicarbonate and extracted with ethyl acetate (20 mL×2). The combined organic layer was dried over Na₂SO₄, filtered and concentrated under reduced pressure to afford N-(3-((tert-butyldimethylsilyl)oxy)-6-chloro-2-methylphenyl)-2-((4-(3-(dimethylamino)propoxy)-3-methylphenyl)amino)-4-methoxypyrimidine-5-carboxamide (120 mg, crude) as a yellow sticky solid. This material was used as such without further purification. Chemical Formula: C₃₁H₄ClN₅O₄Si, Exact Mass: 613.3, Molecular Weight: 614.3. LCMS (ESI) m/z=614.9/616.5/617.5 (M+H), Chlorine isotopes, t_{R}=1.85 min, 79% (Method-A).

### N-(6-chloro-3-hydroxy-2-methylphenyl)-2-((4-(3-(dimethylamino)propoxy)-3-methylphenyl)amino)-4-methoxypyrimidine-5-carboxamide (Compound 158)

To a stirred solution of N-(3-((tert-butyldimethylsilyl)oxy)-6-chloro-2-methylphenyl)-2-((4-(3-(dimethylamino)propoxy)-3-methylphenyl)amino)-4-methoxypyrimidine-5-carboxamide (120 mg, 0.195 mmol) in methanol (2 mL), was added potassium hydrogen fluoride (38.1 mg, 0.488 mmol) at 0° C. The resulting reaction mixture was stirred at room temperature for 1 h. After completion of the reaction (TLC and UPLC), the reaction mixture was concentrated under reduced pressure. The resulting crude material was purified by preparative HPLC to afford N-(6-chloro-3-hydroxy-2-methylphenyl)-2-((4-(3-(dimethylamino)propoxy)-3-methylphenyl)amino)-4-methoxypyrimidine-5-carboxamide (27 mg, 27% yield) as a light yellow solid. ¹H-NMR (400 MHz, DMSO-d₆): δ 9.87 (s, 1H), 9.70 (s, 1H), 9.26 (s, 1H), 8.70 (s, 1H), 7.53-7.50 (m, 2H), 7.16 (d, J=8.40 Hz, 1H), 6.89 (d, J=9.20 Hz, 1H), 6.80 (d, J=8.80 Hz, 1H), 4.08 (s, 3H), 3.98 (t, J=6.00 Hz, 2H), 2.38 (t, J=7.20 Hz, 2H), 2.16-2.15 (m, 9H), 2.03 (s, 3H), 1.85 (q, J=6.80 Hz, 2H). Chemical Formula: C₂₅H₃₀C₁N₅O₄, Exact Mass: 499.2, Molecular Weight: 500.0. LCMS (ESI) m/z=500.4/502.2 (M+H), Chlorine isotopes, t_{R}=1.381 min, 98.5% (Method-C), HPLC purity: 97.0%; t_{R}=5.303 min (Method-B).

### Example 44. Synthesis of N-(6-chloro-3-hydroxy-2-methylphenyl)-2-((4-(2-(dimethylamino)ethoxy)-3-methylphenyl)amino)-4-methoxypyrimidine-5-carboxamide (Compound 149)

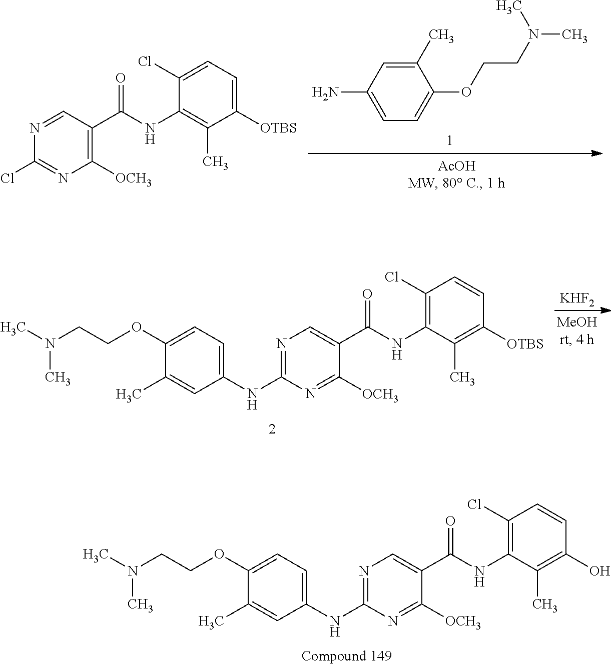 [SMILES: COc1nc(Cl)ncc1C(=O)Nc1c(Cl)ccc(O[Si](C)(C)C(C)(C)C)c1C ; Cc1cc(N)ccc1OCCN(C)C ; COc1nc(Nc2ccc(OCCN(C)C)c(C)c2)ncc1C(=O)Nc1c(Cl)ccc(O[Si](C)(C)C(C)(C)C)c1C ; "KHF2" ; COc1nc(Nc2ccc(OCCN(C)C)c(C)c2)ncc1C(=O)Nc1c(Cl)ccc(O)c1C]

### N-(3-((tert-butyldimethylsilyl)oxy)-6-chloro-2-methylphenyl)-2-((4-(2-(dimethylamino)ethoxy)-3-methylphenyl)amino)-4-methoxypyrimidine-5-carboxamide

To a stirred solution of N-(3-((tert-butyldimethylsilyl)oxy)-6-chloro-2-methylphenyl)-2-chloro-4-methoxypyrimidine-5-carboxamide (120 mg, 0.271 mmol) was added 4-(2-(dimethylamino)ethoxy)-3-methylaniline (58.0 mg, 0.298 mmol) and the reaction mixture was stirred at 80° C. for 1 h under microwave irradiation to afford N-(3-((tert-butyldimethylsilyl)oxy)-6-chloro-2-methylphenyl)-2-((4-(2-(dimethylamino)ethoxy)-3-methylphenyl)amino)-4-methoxypyrimidine-5-carboxamide (170 mg, crude) as a pale yellow solid. Chemical Formula: C₃₀H₄₂ClN₅O₄Si, Exact Mass: 599.3, Molecular Weight: 600.2. LCMS (EST) m/z=601.5/603.4 (M+H), Chlorine isotopes, t_{R}=2.5 min, 36.4% (Method-C).

### N-(6-chloro-3-hydroxy-2-methylphenyl)-2-((4-(2-(dimethylamino)ethoxy)-3-methylphenyl)amino)-4-methoxypyrimidine-5-carboxamide (Compound 149)

To a stirred solution of N-(3-((tert-butyldimethylsilyl)oxy)-6-chloro-2-methylphenyl)-2-((4-(2-(dimethylamino)ethoxy)-3-methylphenyl)amino)-4-methoxypyrimidine-5-carboxamide (170 mg, 0.283 mmol) was added potassium hydrogen fluoride (33.2 mg, 0.425 mmol) and the reaction mixture was stirred at room temperature for 4 h. The resultant crude residue was purified by prep. HPLC to afford N-(6-chloro-3-hydroxy-2-methylphenyl)-2-((4-(2-(dimethylamino)ethoxy)-3-methylphenyl)amino)-4-methoxypyrimidine-5-carboxamide (10 mg, 7% yield) as an off-white solid. ¹H-NMR (400 MHz, DMSO-d₆): δ 9.88 (s, 1H), 9.70 (s, 1H), 9.26 (s, 1H), 8.71 (s, 1H), 7.54-7.52 (m, 2H), 7.17 (d, J=8.80 Hz, 1H), 6.92 (d, J=9.20 Hz, 1H), 6.80 (d, J=8.40 Hz, 1H), 4.09 (s, 3H), 4.04 (t, J=6.00 Hz, 2H), 2.67-2.64 (m, 2H), 2.24 (s, 6H), 2.16 (s, 3H), 2.03 (s, 3H). Chemical Formula: C₂₄H₂₈C₁N₅O₄, Exact Mass: 485.2, Molecular Weight: 486.0. LCMS (ESI) m/z=486.3 (M+H), t_{R}=1.883 min, 98.8% (Method-B), HPLC purity: 98.9%; t_{R}=3.376 min (Method-A).

### Example 45. Synthesis of N-(6-chloro-3-hydroxy-2-methylphenyl)-4-methoxy-2-((3-methyl-4-(3-morpholinopropoxy)phenyl)amino)pyrimidine-5-carboxamide (Compound 150)

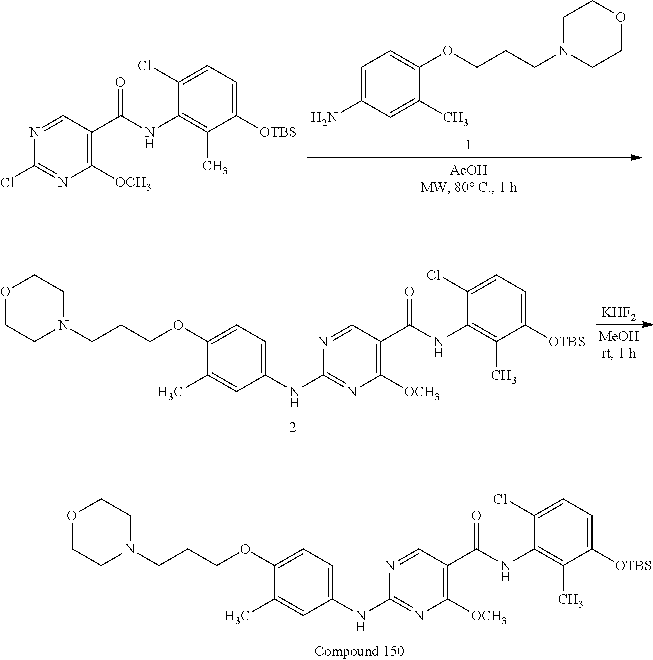 [SMILES: COc1nc(Cl)ncc1C(=O)Nc1c(Cl)ccc(O[Si](C)(C)C(C)(C)C)c1C ; Cc1cc(N)ccc1OCCCN1CCOCC1 ; COc1nc(Nc2ccc(OCCCN3CCOCC3)c(C)c2)ncc1C(=O)Nc1c(Cl)ccc(O[Si](C)(C)C(C)(C)C)c1C ; "KHF2" ; COc1nc(Nc2ccc(OCCCN3CCOCC3)c(C)c2)ncc1C(=O)Nc1c(Cl)ccc(O[Si](C)(C)C(C)(C)C)c1C]

### N-(3-((tert-butyldimethylsilyl)oxy)-6-chloro-2-methylphenyl)-4-methoxy-2-((3-methyl-4-(3-morpholinopropoxy)phenyl)amino)pyrimidine-5-carboxamide

To a stirred solution of N-(3-((tert-butyldimethylsilyl)oxy)-6-chloro-2-methylphenyl)-2-chloro-4-methoxypyrimidine-5-carboxamide (0.120 g, 0.271 mmol) was added 3-methyl-4-(3-morpholinopropoxy)aniline (0.068 g, 0.271 mmol) and the reaction mixture was stirred at 80° C. for 1 h under microwave irradiation to afford N-(3-((tert-butyldimethylsilyl)oxy)-6-chloro-2-methylphenyl)-4-methoxy-2-((3-methyl-4-(3-morpholinopropoxy)phenyl)amino) pyrimidine-5-carboxamide (194 mg, crude) as a pale yellow solid. The crude material was as such used for the next step without further purification. Chemical Formula: C₃₃H₄₆ClN₅O₅Si, Exact Mass: 655.3, Molecular Weight: 656.3. LCMS (ESI) m/z=657.0/658.5 (M+H), Chlorine isotopes, t_{R}=1.84 min, 72.3% (Method-A).

### N-(6-chloro-3-hydroxy-2-methylphenyl)-4-methoxy-2-((3-methyl-4-(3-morpholinopropoxy)phenyl)amino)pyrimidine-5-carboxamide (Compound 150)

To a stirred solution of N-(3-((tert-butyldimethylsilyl)oxy)-6-chloro-2-methylphenyl)-4-methoxy-2-((3-methyl-4-(3-morpholinopropoxy)phenyl)amino)pyrimidine-5-carboxamide (0.194 g, 0.296 mmol) was added potassium hydrogen fluoride (0.023 g, 0.296 mmol) as reactants and the reaction mixture was stirred at room temperature for 1 h. The resultant crude residue was purified by prep. HPLC to afford N-(6-chloro-3-hydroxy-2-methylphenyl)-4-methoxy-2-((3-methyl-4-(3-morpholinopropoxy)phenyl)amino)pyrimidine-5-carboxamide (0.047 g, 29% yield) as an off-white solid. ¹H-NMR (400 MHz, DMSO-d₆): δ 9.88 (s, 1H), 9.70 (s, 1H), 9.26 (s, 1H), 8.70 (s, 1H), 7.53-7.52 (m, 2H), 7.17 (d, J=8.40 Hz, 1H), 6.90 (d, J=4.00 Hz, 1H), 6.80 (d, J=8.80 Hz, 1H), 4.08 (s, 3H), 3.99 (t, J=6.40 Hz, 2H), 3.58 (t, J=4.80 Hz, 4H), 2.47-2.45 (m, 2H), 2.42-2.35 (m, 4H), 2.16 (s, 3H), 2.03 (s, 3H), 1.89 (q, J=6.80 Hz, 2H). Chemical Formula: C₂₇H₃₂ClN₅O₅, Exact Mass: 541.2, Molecular Weight: 542.0. LCMS (ESI) m/z=542.4 (M+H), t_{R}=2.107 min, 99.5% (Method-B), HPLC purity: 99.4%; t_{R}=3.575 min (Method-A).

### Example 46. Synthesis of N-(6-chloro-3-hydroxy-2-methylphenyl)-4-methoxy-2-((3-methyl-4-(2-morpholinoethoxy)phenyl)amino)pyrimidine-5-carboxamide (Compound 151)

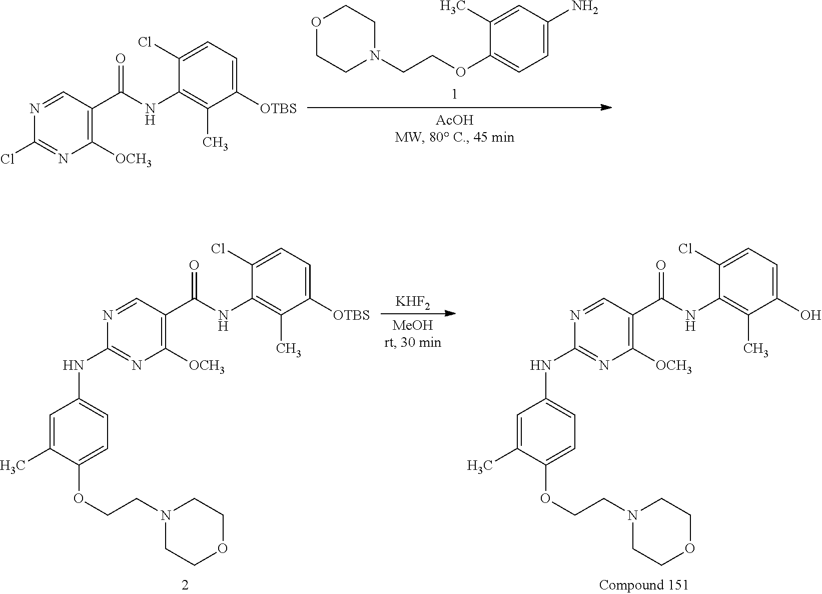 [SMILES: COc1nc(Cl)ncc1C(=O)Nc1c(Cl)ccc(O[Si](C)(C)C(C)(C)C)c1C ; Cc1cc(N)ccc1OCCN1CCOCC1 ; COc1nc(Nc2ccc(OCCN3CCOCC3)c(C)c2)ncc1C(=O)Nc1c(Cl)ccc(O[Si](C)(C)C(C)(C)C)c1C ; "KHF2" ; COc1nc(Nc2ccc(OCCN3CCOCC3)c(C)c2)ncc1C(=O)Nc1c(Cl)ccc(O)c1C]

### N-(3-((tert-butyldimethylsilyl)oxy)-6-chloro-2-methylphenyl)-4-methoxy-2-((3-methyl-4-(2-morpholinoethoxy)phenyl)amino)pyrimidine-5-carboxamide

To a stirred solution of N-(3-((tert-butyldimethylsilyl)oxy)-6-chloro-2-methylphenyl)-2-chloro-4-methoxypyrimidine-5-carboxamide (100 mg, 0.226 mmol) was added 3-methyl-4-(2-morpholinoethoxy)aniline (80 mg, 0.339 mmol) and the reaction mixture was stirred at 80° C. for 45 minutes under microwave irradiation to afford N-(3-((tert-butyldimethylsilyl)oxy)-6-chloro-2-methylphenyl)-4-methoxy-2-((3-methyl-4-(2-morpholinoethoxy)phenyl)amino) pyrimidine-5-carboxamide (100 mg, crude) as a pale yellow solid. The crude material was as such used for the next step without further purification. Chemical Formula: C₃₂H₄₄ClNO₅Si, Exact Mass: 641.3, Molecular Weight: 642.3. LCMS (ESI) m/z=642.9/643.7 (M+H), Chlorine isotopes, t_{R}=2.62 min, 66% (Method-C).

### N-(6-chloro-3-hydroxy-2-methylphenyl)-4-methoxy-2-((3-methyl-4-(2-morpholinoethoxy)phenyl)amino)pyrimidine-5-carboxamide (Compound 151)

To a stirred solution of N-(3-((tert-butyldimethylsilyl)oxy)-6-chloro-2-methylphenyl)-4-methoxy-2-((3-methyl-4-(2-morpholinoethoxy)phenyl)amino)pyrimidine-5-carboxamide (100 mg, 0.156 mmol) was added potassium hydrogen fluoride (24.32 mg, 0.311 mmol) and the reaction mixture was stirred at room temperature for 30 minutes to afford N-(6-chloro-3-hydroxy-2-methylphenyl)-4-methoxy-2-((3-methyl-4-(2-morpholinoethoxy)phenyl)amino) pyrimidine-5-carboxamide (11 mg, 13% yield) as an off-white solid. ¹H-NMR (400 MHz, DMSO-d₆): δ 9.88 (s, 1H), 9.70 (s, 1H), 9.26 (s, 1H), 8.71 (s, 1H), 7.53-7.51 (m, 2H), 7.17 (d, J=8.80 Hz, 1H), 6.93 (d, J=8.80 Hz, 1H), 6.80 (d, J=8.40 Hz, 1H), 4.08-4.06 (m, 5H), 3.59 (t, J=4.80 Hz, 4H), 2.72 (t, J=6.00 Hz, 2H), 2.16 (s, 3H), 2.03 (s, 3H). Note: 4 Protons merged in solvent peak. Chemical Formula: C₂₆H₃₀C₁N₅O₅, Exact Mass: 527.2, Molecular Weight: 528.0. LCMS (ESI) m/z=529.2/530.2 (M+H), chlorine isotopes, t_{R}=1.248 min, 99.7% (Method-A), HPLC purity: 99.8%; t_{R}=3.497 min (Method-A).

### Example 47. Synthesis of N-(6-chloro-3-hydroxy-2-methylphenyl)-4-methoxy-2-((3-methyl-4-((1-methylpiperidin-4-yl)oxy)phenyl)amino)pyrimidine-5-carboxamide (Compound 152)

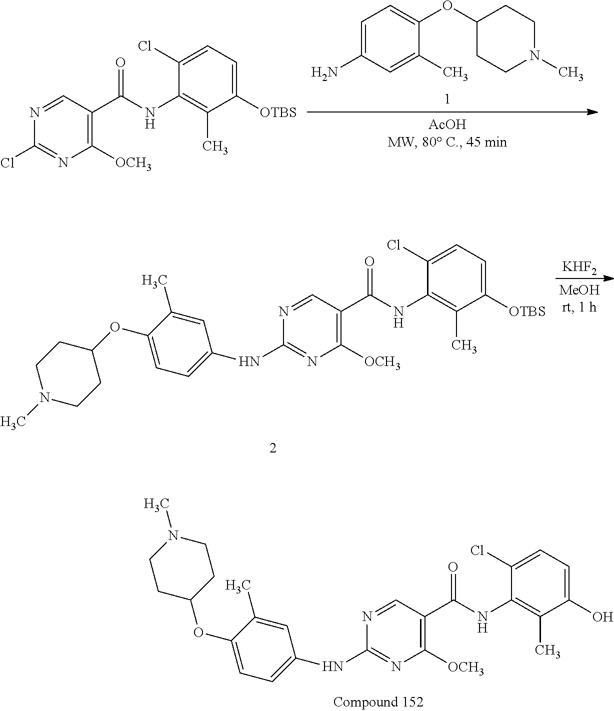 [SMILES: COc1nc(Cl)ncc1C(=O)Nc1c(Cl)ccc(O[Si](C)(C)C(C)(C)C)c1C ; Cc1cc(N)ccc1OC1CCN(C)CC1 ; COc1nc(Nc2ccc(OC3CCN(C)CC3)c(C)c2)ncc1C(=O)Nc1c(Cl)ccc(O[Si](C)(C)C(C)(C)C)c1C ; "KHF2" ; COc1nc(Nc2ccc(OC3CCN(C)CC3)c(C)c2)ncc1C(=O)Nc1c(Cl)ccc(O)c1C]

### N-(3-((tert-butyldimethylsilyl)oxy)-6-chloro-2-methylphenyl)-4-methoxy-2-((3-methyl-4-((1-methylpiperidin-4-yl)oxy)phenyl)amino)pyrimidine-5-carboxamide

To a stirred solution of N-(3-((tert-butyldimethylsilyl)oxy)-6-chloro-2-methylphenyl)-2-chloro-4-methoxypyrimidine-5-carboxamide (120 mg, 0.271 mmol) was added 3-methyl-4-((1-methylpiperidin-4-yl)oxy)aniline (65.7 mg, 0.298 mmol) and the reaction mixture was stirred at 80° C. for 45 minutes under microwave irradiation to afford N-(3-((tert-butyldimethylsilyl)oxy)-6-chloro-2-methylphenyl)-4-methoxy-2-((3-methyl-4-((1-methylpiperidin-4-yl)oxy)phenyl)amino)pyrimidine-5-carboxamide (150 mg, crude) as a pale brown solid. The crude material was as such used for the next step without further purification. Chemical Formula: C₃₂H₄₄ClN₅O₄Si, Exact Mass: 625.3, Molecular Weight: 626.3. LCMS (ESI) m/z=627.5/629.5 (M+H), Chlorine isotopes, t_{R}=1.79 min, 79% (Method-A).

### N-(6-chloro-3-hydroxy-2-methylphenyl)-4-methoxy-2-((3-methyl-4-((I-methylpiperidin-4-yl)oxy)phenyl)amino)pyrimidine-5-carboxamide (Compound 152)

To a stirred solution of N-(3-((tert-butyldimethylsilyl)oxy)-6-chloro-2-methylphenyl)-4-methoxy-2-((3-methyl-4-((1-methylpiperidin-4-yl)oxy)phenyl)amino)pyrimidine-5-carboxamide (150 mg, 0.240 mmol) was added potassium hydrogen fluoride (28.1 mg, 0.359 mmol) and the reaction mixture was stirred at room temperature for 1 h. The resultant crude residue was purified by prep. HPLC to afford N-(6-chloro-3-hydroxy-2-methylphenyl)-4-methoxy-2-((3-methyl-4-((1-methylpiperidin-4-yl)oxy)phenyl)amino)pyrimidine-5-carboxamide (50 mg, 40% yield) as an off-white solid. ¹H-NMR (400 MHz, DMSO-d₆): δ 9.88 (s, 1H), 9.71 (s, 1H), 9.26 (s, 1H), 8.71 (s, 1H), 7.54-7.50 (m, 2H), 7.17 (d, J=8.80 Hz, 1H), 6.94 (d, J=8.80 Hz, 1H), 6.80 (d, J=8.80 Hz, 1H), 4.32-4.30 (m, 1H), 4.09 (s, 3H), 2.56-2.53 (m, 2H), 2.22-2.17 (m, 8H), 2.03 (s, 3H), 1.93-1.88 (m, 2H), 1.71-1.64 (m, 2H). Chemical Formula: C₂₆H₃₀ClN₅O₄, Exact Mass: 511.2, Molecular Weight: 512.0. LCMS (ESI) m/z=512.4/513.2/514.2 (M+H), Chlorine isotopes, t_{R}=1.669 min, 99.8% (Method-C), HPLC purity: 99.4%; t_{R}=3.575 min (Method-A).

### Example 48. Synthesis of (S)—N-(6-chloro-3-hydroxy-2-methylphenyl)-4-methoxy-2-((3-methyl-4-((1-methylpyrrolidin-3-yl)oxy)phenyl)amino)pyrimidine-5-carboxamide (Compound 155)

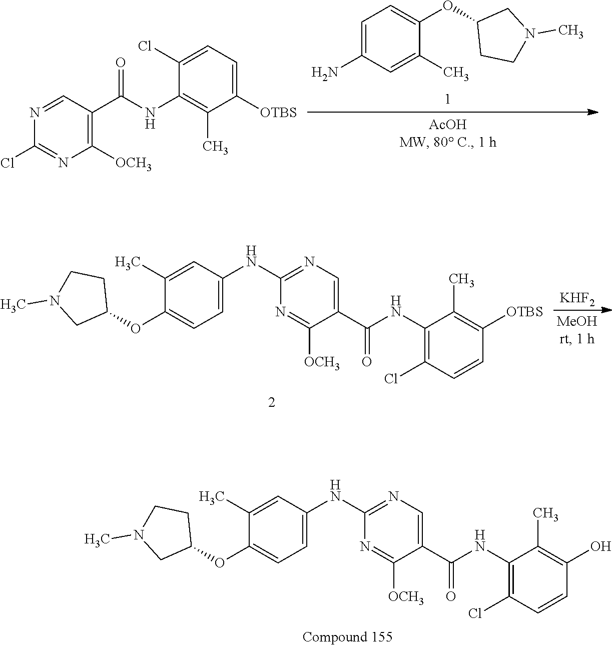

### (S)—N-(3-((tert-butyldimethylsilyl)oxy)-6-chloro-2-methylphenyl)-4-methoxy-2-((3-methyl-4-((1-methylpyrrolidin-3-yl)oxy)phenyl)amino)pyrimidine-5-carboxamide

To a stirred solution of N-(3-((tert-butyldimethylsilyl)oxy)-6-chloro-2-methylphenyl)-2-chloro-4-methoxypyrimidine-5-carboxamide (100 mg, 0.226 mmol) was added (S)-3-methyl-4-((1-methylpyrrolidin-3-yl)oxy)aniline (93 mg, 0.452 mmol) and the reaction mixture was stirred at 80° C. for 1 h under microwave irradiation to afford (S)—N-(3-((tert-butyldimethylsilyl)oxy)-6-chloro-2-methylphenyl)-4-methoxy-2-((3-methyl-4-((1-methylpyrrolidin-3-yl)oxy)phenyl)amino)pyrimidine-5-carboxamide (100 mg, crude) as a pale yellow solid. The crude material as such used for the next step without further purification. Chemical Formula: C₃₁H₄₂C₁N₅O₄Si, Exact Mass: 611.3, Molecular Weight: 612.2. LCMS (ESI) m/z=613.7/614.7 (M+H), Chlorine isotopes, t_{R}=1.8 min, 50% (Method-A).

### (S)—N-(6-chloro-3-hydroxy-2-methylphenyl)-4-methoxy-2-((3-methyl-4-((1-methylpyrrolidin-3-yl)oxy)phenyl)amino)pyrimidine-5-carboxamide (Compound 155)

To a stirred solution of (S)—N-(3-((tert-butyldimethylsilyl)oxy)-6-chloro-2-methylphenyl)-4-methoxy-2-((3-methyl-4-((1-methylpyrrolidin-3-yl)oxy)phenyl)amino)pyrimidine-5-carboxamide (100 mg, 0.163 mmol) was added KHF₂ (12.76 mg, 0.163 mmol) and the reaction mixture was stirred at room temperature for 1 h. The resultant crude residue was purified by prep. HPLC to afford (S)—N-(6-chloro-3-hydroxy-2-methylphenyl)-4-methoxy-2-((3-methyl-4-((1-methylpyrrolidin-3-yl)oxy)phenyl)amino)pyrimidine-5-carboxamide (7 mg, 9% yield) as an off-white solid. ¹H-NMR (400 MHz, DMSO-d₆): δ 9.88 (s, 1H), 9.70 (s, 1H), 9.26 (s, 1H), 8.70 (s, 1H), 7.54-7.49 (m, 2H), 7.17 (d, J=8.80 Hz, 1H), 6.84-6.79 (m, 2H), 4.83-4.80 (m, 1H), 4.09 (s, 3H), 2.83-2.79 (m, 1H), 2.65-2.57 (m, 2H), 2.42-2.36 (m, 1H), 2.31-2.25 (m, 4H), 2.14 (s, 3H), 2.03 (s, 3H), 1.82-1.79 (m, 1H). Chemical Formula: C₂₅H₂₈C₁N₅O₄, Exact Mass: 497.2, Molecular Weight: 498.0. LCMS (ESI) m/z=498.3 (M+H), t_{R}=1.883 min, 97.4% (Method-B), HPLC purity: 96.1%; t_{R}=3.491 min (Method-A).

### Example 49. Synthesis of (R)—N-(6-chloro-3-hydroxy-2-methylphenyl)-4-methoxy-2-((3-methyl-4-((1-methylpyrrolidin-3-yl)oxy)phenyl)amino)pyrimidine-5-carboxamide

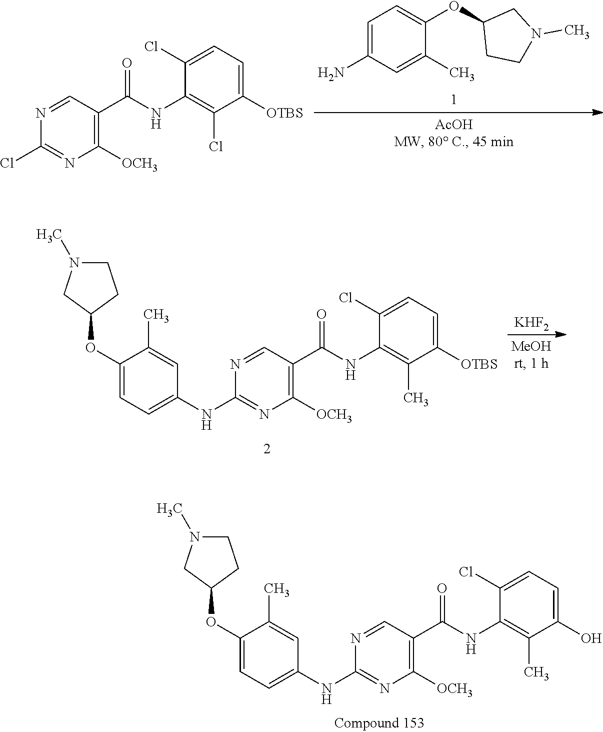 [SMILES: COc1nc(Cl)ncc1C(=O)Nc1c(Cl)ccc(O[Si](C)(C)C(C)(C)C)c1Cl ; Cc1cc(N)ccc1O[C@@H]1CCN(C)C1 ; COc1nc(Nc2ccc(O[C@@H]3CCN(C)C3)c(C)c2)ncc1C(=O)Nc1c(Cl)ccc(O[Si](C)(C)C(C)(C)C)c1C ; "KHF2" ; COc1nc(Nc2ccc(O[C@@H]3CCN(C)C3)c(C)c2)ncc1C(=O)Nc1c(Cl)ccc(O)c1C]

### (R)—N-(3-((tert-butyldimethylsilyl)oxy)-6-chloro-2-methylphenyl)-4-methoxy-2-((3-methyl-4-((1-methylpyrrolidin-3-yl)oxy)phenyl)amino)pyrimidine-5-carboxamide

To a stirred solution of N-(3-((tert-butyldimethylsilyl)oxy)-6-chloro-2-methylphenyl)-2-chloro-4-methoxypyrimidine-5-carboxamide (0.120 g, 0.271 mmol) was added (R)-3-methyl-4-((1-methylpyrrolidin-3-yl)oxy)aniline (0.062 g, 0.298 mmol) and the reaction mixture was stirred at 80° C. for 45 minutes under microwave irradiation to afford (R)—N-(3-((tert-butyldimethylsilyl)oxy)-6-chloro-2-methylphenyl)-4-methoxy-2-((3-methyl-4-((1-methylpyrrolidin-3-yl)oxy)phenyl)amino)pyrimidine-5-carboxamide (0.160 g, crude) as a pale yellow sticky solid. The crude material as such used for the next step without further purification. Chemical Formula: C₃₁H₄₂ClN₅O₄Si, Exact Mass: 611.3, Molecular Weight: 612.2. LCMS (ESI) m/z=613.5/615.5 (M+H), Chlorine isotopes, t_{R}=1.79 min, 70% (Method-A).

### (R)—N-(6-chloro-3-hydroxy-2-methylphenyl)-4-methoxy-2-((3-methyl-4-((1-methylpyrrolidin-3-yl)oxy)phenyl)amino)pyrimidine-5-carboxamide (Compound 153)

To a stirred solution of (R)—N-(3-((tert-butyldimethylsilyl)oxy)-6-chloro-2-methylphenyl)-4-methoxy-2-((3-methyl-4-((1-methylpyrrolidin-3-yl)oxy)phenyl)amino)pyrimidine-5-carboxamide (0.160 g, 0.196 mmol) was added potassium hydrogen fluoride (0.023 g, 0.294 mmol) and the reaction mixture was stirred at room temperature for 1 h. The resultant crude residue was purified by prep. HPLC to afford (R)—N-(6-chloro-3-hydroxy-2-methylphenyl)-4-methoxy-2-((3-methyl-4-((1-methylpyrrolidin-3-yl)oxy)phenyl)amino)pyrimidine-5-carboxamide (0.023 g, 23% yield) as an off-white solid. ¹H-NMR (400 MHz, DMSO-d₆): δ 9.87 (s, 1H), 9.74 (s, 1H), 9.26 (s, 1H), 8.70 (s, 1H), 7.54-7.49 (m, 2H), 7.16 (d, J=8.80 Hz, 1H), 6.81 (t, J=9.20 Hz, 2H), 4.83-4.80 (m, 1H), 4.09 (s, 3H), 4.07-3.33 (m, 1H), 2.69-2.55 (m, 2H), 2.40-2.31 (m, 1H), 2.30-2.22 (m, 4H), 2.14 (s, 3H), 2.03 (s, 3H), 1.82-1.74 (m, 1H). Chemical Formula: C₂₅H₂₈C₁N₅O₄, Exact Mass: 497.2, Molecular Weight: 498.0. LCMS (ESI) m/z=498.3/498.7/500.3 (M+H), Chlorine isotopes, t_{R}=1.616 min, 96.8% (Method-C), HPLC purity: 95.2%; t_{R}=3.508 min (Method-A).

### Example 50. Synthesis of 4-cyclopropoxy-N-(2,6-dichlorophenyl)-2-((3-methyl-4-(1-methylpiperidin-4-yl)phenyl)amino)pyrimidine-5-carboxamide (Compound 140)

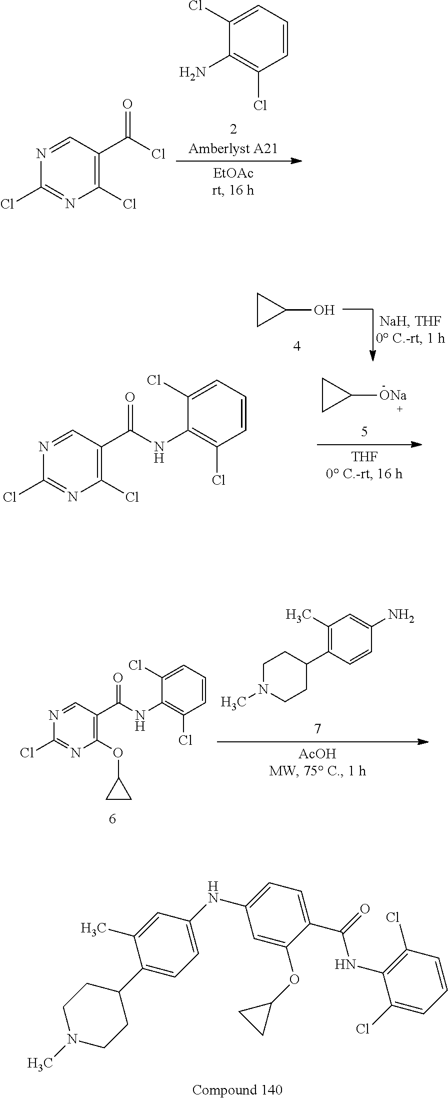

### 2,4-dichloro-N-(2,6-dichlorophenyl)pyrimidine-5-carboxamide

To a stirred solution of 2,4-dichloropyrimidine-5-carbonyl chloride (500 mg, 2.365 mmol) in ethyl acetate (30 mL) was added 2,6-dichloroaniline (421 mg, 2.60 mmol) and Amberlyst A21 (100 mg) at 0° C. The reaction mixture was stirred at room temperature for 16 h. After completion of the reaction (TLC and UPLC), the reaction mixture was diluted with water and extracted with ethyl acetate (20 mL×2). Organic layer was washed with sodium bicarbonate (20 mL). The combined organic layer was dried over Na₂SO₄, filtered and concentrated under reduced pressure to afford 2,4-dichloro-N-(2,6-dichlorophenyl)pyrimidine-5-carboxamide (350 mg, 44% yield) as an off white solid. ¹H-NMR (400 MHz, DMSO-d₆): δ 10.85 (s, 1H), 9.02 (s, 1H), 7.63 (d, J=8.40 Hz, 2H), 7.47-7.43 (m, 1H). Chemical Formula: C_{II}H₅Cl₄N₃O, Exact Mass: 334.9, Molecular Weight: 337.0

### 2-chloro-4-cyclopropoxy-N-(2,6-dichlorophenyl)pyrimidine-5-carboxamide

To a stirred solution of NaH (35.6 mg, 0.890 mmol) in THF (5 mL) was added cyclopropanol (51.7 mg, 0.890 mmol) at 0° C. The reaction mixture was stirred at room temperature for 1 h. The resulting reaction mixture was added to the stirred solution of 2,4-dichloro-N-(2,6-dichlorophenyl) pyrimidine-5-carboxamide (250 mg, 0.742 mmol) in THF (5 mL) at 0° C. The reaction mixture was stirred at room temperature for 16 h. After completion of the reaction (TLC and UPLC), the reaction mixture was diluted with water and extracted with ethyl acetate (20 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 2-chloro-4-cyclopropoxy-N-(2,6-dichlorophenyl) pyrimidine-5-carboxamide (50 mg, 19% yield) as a yellow solid. ¹H-NMR (400 MHz, DMSO-d₆): δ 10.08 (s, 1H), 8.76 (s, 1H), 7.60 (d, J=8.00 Hz, 2H), 7.44-7.40 (m, 1H), 4.56-4.52 (m, 1H), 0.92-0.88 (m, 4H). Note: Regioselectivity was confirmed by 1D nOe. Chemical Formula: C₁₄H₁₀Cl₃N₃O₂, Exact Mass: 357.0, Molecular Weight: 358.6. LCMS (ESI) m/z=358.0 (M+H), t_{R}=2.585 min, 99.4% (Method-A).

### 4-cyclopropoxy-N-(2,6-dichlorophenyl)-2-((3-methyl-4-(1-methylpiperidin-4-yl)phenyl)amino)pyrimidine-5-carboxamide (Compound 140)

To a stirred solution of 2-chloro-4-cyclopropoxy-N-(2,6-dichlorophenyl) pyrimidine-5-carboxamide (40 mg, 0.112 mmol) was added 3-methyl-4-(1-methyl piperidin-4-yl)aniline (22.79 mg, 0.112 mmol) and the reaction mixture was stirred at 75° C. for 1 h under microwave irradiation. The resulting crude was purified by prep-HPLC to afford 4-cyclopropoxy-N-(2,6-dichlorophenyl)-2-((3-methyl-4-(1-methylpiperidin-4-yl)phenyl)amino)pyrimidine-5-carboxamide (14 mg, 24% yield) as an off white solid. ¹H-NMR (400 MHz, DMSO-d₆): δ 10.05 (s, 1H), 9.28 (s, 1H), 8.69 (s, 1H), 7.66-7.62 (m, 2H), 7.57 (d, J=8.00 Hz, 2H), 7.40-7.36 (m, 1H), 7.17 (d, J=8.40 Hz, 1H), 4.58-4.50 (m, 1H), 2.90-2.85 (m, 2H), 2.63-2.58 (m, 1H), 2.29 (s, 3H), 2.21 (s, 3H), 2.05-1.95 (m, 2H), 1.69-1.60 (m, 4H), 1.04-1.00 (m, 2H), 0.91-0.87 (m, 2H). Chemical Formula: C₂₇H₂₉Cl₂N₅O₂, Exact Mass: 525.2, Molecular Weight: 526.5. LCMS (ESI) im/z=526.2/528.2/530.2 (M+H), chlorine isotopes, t_{R}=2.178 min, 99.2% (Method-A), HPLC purity: 95.8%; t_{R}=6.375 min (Method-B).

### Example 51. Synthesis of N-(3-hydroxy-2,6-dimethylphenyl)-4-methoxy-2-((3-methyl-4-(1-methyl piperidin-4-yl)phenyl)amino)pyrimidine-5-carboxamide (Compound 142)

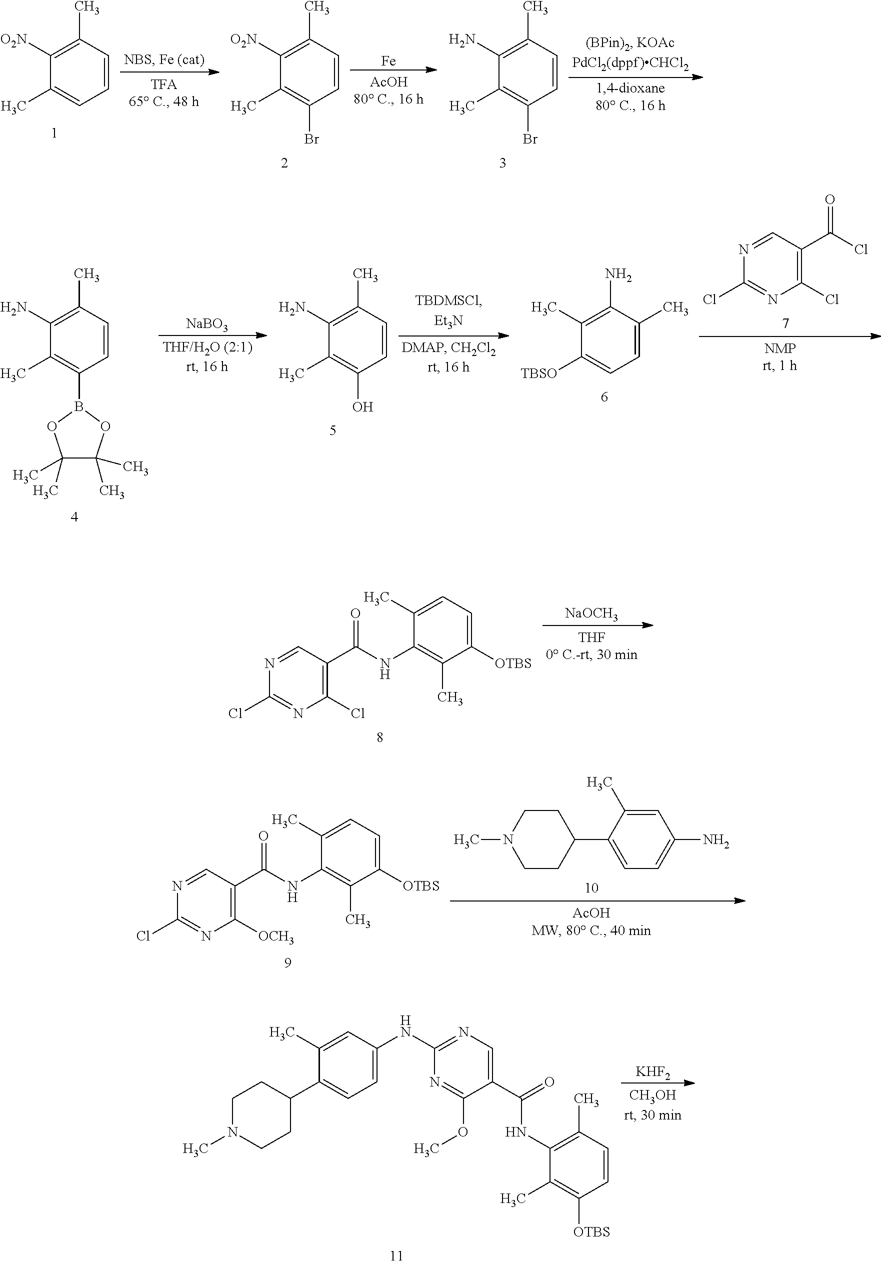

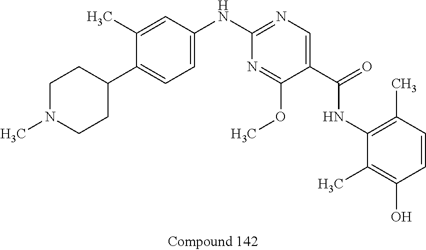 [SMILES: COc1nc(Nc2ccc(C3CCN(C)CC3)c(C)c2)ncc1C(=O)Nc1c(C)ccc(O)c1C]

### 1-bromo-2,4-dimethyl-3-nitrobenzene

To a stirred solution of 1,3-dimethyl-2-nitrobenzene (5 g, 33.1 mmol) in TFA (40 mL) were added iron (0.369 g, 6.62 mmol) and NBS (23.55 g, 132 mmol) at 0° C. The reaction mixture was stirred at 65° C. for 48 h. After completion of the reaction (TLC and UPLC), the reaction mixture was concentrated under reduced pressure. The resultant crude was basified with aqueous sodium bicarbonate and extracted with ethyl acetate (20 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 1-bromo-2,4-dimethyl-3-nitrobenzene (6 g, 47% yield) as an off white solid. ¹H-NMR (400 MHz, CDCl₃): δ 7.57 (d, J=8.00 Hz, 1H), 7.05-7.02 (m, 1H), 2.37 (s, 3H), 2.27 (s, 3H). Chemical Formula: C₈H₈BrNO₂, Exact Mass: 229.0, Molecular Weight: 230.1. Product was not ionised in LCMS.

### 3-bromo-2,6-dimethylaniline

To a stirred solution of 1-bromo-2,4-dimethyl-3-nitrobenzene (3 g, 13.04 mmol) in AcOH (24 mL) was added iron (2.91 g, 52.2 mmol) at room temperature. The reaction mixture was stirred at 80° C. for 16 h. After completion of the reaction (TLC and UPLC), the reaction mixture was filtered through celite, the filtrate was basified with ammonium bicarbonate solution and extracted with ethyl acetate (30 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 3-bromo-2,6-dimethylaniline (2 g, 77% yield) as a colourless liquid. Chemical Formula: C₈H₁₀BrN, Exact Mass: 199.0, Molecular Weight: 200.1. LCMS (ESI) m/z=200.0/202.0 (M+H), bromine isotopes, t_{R}=2.381 min, 47% (Method-A).

### 2,6-dimethyl-3-(4,4,5,5-tetramethyl-1,3,2-dioxaborolan-2-yl)aniline

To a stirred solution of 3-bromo-2,6-dimethylaniline (4 g, 19.99 mmol) and bis(pinacolato)diboron (6.09 g, 23.99 mmol) in 1,4-dioxane (40 mL) was added potassium acetate (5.89 g, 60.0 mmol) at room temperature. After degassing with nitrogen for 5 min, 1,1′-bis(diphenylphosphino)ferrocene dichloro palladium(II) dichloromethane complex (0.878 g, 1.200 mmol) was added to the reaction mixture under nitrogen atmosphere. The resulting reaction mixture was stirred at 80° C. for 16 h. After completion of the reaction (TLC and UPLC), the reaction mixture was filtered through celite, the filtrate was diluted with water and extracted with ethyl acetate (30 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 2,6-dimethyl-3-(4,4,5,5-tetramethyl-1,3,2-dioxaborolan-2-yl)aniline (2.5 g, 33% yield) as a yellow sticky solid. Chemical Formula: C₁₄H₂₂BNO₂, Exact Mass: 247.2, Molecular Weight: 247.1. LCMS (ESI) m/z=248.3 (M+H), t_{R}=2.417 min, 66.1% (Method-A).

### 3-amino-2,4-dimethylphenol

To a stirred solution of 2,6-dimethyl-3-(4,4,5,5-tetramethyl-1,3,2-dioxaborolan-2-yl)aniline (1.5 g, 6.07 mmol) in THF (30 mL) and water (15 mL), was added a sodium perborate tetrahydrate (156 mg, 1.012 mmol) at 0° C. The resulting reaction mixture was stirred at room temperature for 16 h. After completion of the reaction (TLC and UPLC), the reaction mixture was diluted with water and extracted with ethyl acetate (50 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford 3-amino-2,4-dimethylphenol (300 mg, 36% yield) as an orange solid. ¹H-NMR (400 MHz, DMSO-d₆): δ 8.57 (s, 1H), 6.55 (d, J=8.00 Hz, 1H), 6.02 (d, J=8.00 Hz, 1H), 4.36 (s, 2H), 1.97 (s, 3H), 1.90 (s, 3H). Chemical Formula: C₈H₁₁NO, Exact Mass: 137.1, Molecular Weight: 137.2. LCMS (ESI) m/z=138.2 (M+H), t_{R}=1.077 min, 91.8% (Method-B).

### 3-((tert-butyldimethylsilyl)oxy)-2,6-dimethylaniline

To a stirred solution of 3-amino-2,4-dimethylphenol (450 mg, 3.28 mmol) was added TBDMSCl (1483 mg, 9.84 mmol) as reactants and the reaction mixture was stirred at room temperature for 16 h to afford 3-((tert-butyldimethylsilyl)oxy)-2,6-dimethylaniline (350 mg, 42% yield) as a brown solid. ¹H-NMR (400 MHz, DMSO-d₆): δ 6.64 (d, J=8.00 Hz, 1H), 6.02 (d, J=8.00 Hz, 1H), 4.48 (s, 2H), 2.00 (s, 3H), 1.94 (s, 3H), 1.01 (s, 9H), 0.13 (s, 6H). Chemical Formula: C₁₄H₂₅NOSi, Exact Mass: 251.2, Molecular Weight: 251.4. LCMS (ESI) m/z=252.7 (M+2H), t_{R}=2.33 min, 93% (Method-A).

### N-(3-((tert-butyldimethylsilyl)oxy)-2,6-dimethylphenyl)-2,4-dichloropyrimidine-5-carboxamide

To a stirred solution of 3-((tert-butyldimethylsilyl)oxy)-2,6-dimethylaniline (350 mg, 1.392 mmol) was added 2,4-dichloropyrimidine-5-carbonyl chloride (353 mg, 1.670 mmol) and the reaction mixture was stirred at room temperature for 1 h to afford N-(3-((tert-butyldimethylsilyl)oxy)-2,6-dimethylphenyl)-2,4-dichloropyrimidine-5-carboxamide (250 mg, 40% yield) as a colourless gum. ¹H-NMR (400 MHz, DMSO-d₆): δ 10.17 (s, 1H), 9.09 (s, 1H), 7.01 (d, J=8.00 Hz, 1H), 6.75 (d, J=8.00 Hz, 1H), 2.19 (s, 3H), 2.12 (s, 3H), 1.00 (s, 9H), 0.21 (s, 6H). Chemical Formula: C₁₉H₂₅Cl₂N₃O₂Si, Exact Mass: 425.1, Molecular Weight: 426.4. LCMS (ESI) m/z=426.6/428.3/429.3/431.3 (M+H), chlorine isotopes, t_{R}=2.31 min, 96% (Method-A).

### N-(3-((tert-butyldimethylsilyl)oxy)-2,6-dimethylphenyl)-2-chloro-4-methoxypyrimidine-5-carboxamide

To a stirred solution of N-(3-((tert-butyl dimethylsilyl)oxy)-2,6-dimethylphenyl)-2,4-dichloropyrimidine-5-carboxamide (250 mg, 0.586 mmol) was added sodium methoxide (47.5 mg, 0.879 mmol) and the reaction mixture was stirred at room temperature for 30 minutes to afford N-(3-((tert-butyldimethylsilyl)oxy)-2,6-dimethylphenyl)-2-chloro-4-methoxypyrimidine-5-carboxamide (60 mg, 24% yield) as an off white solid. Chemical Formula: C₂₀H₂₈ClN₃O₃Si, Exact Mass: 421.2, Molecular Weight: 422.0. LCMS (ESI) m/z=422.1/423.1/424.1 (M+H), chlorine isotopes, t_{R}=2.549 min, 33.8% (Method-A).

### N-(3-((tert-butyldimethylsilyl)oxy)-2,6-dimethylphenyl)-4-methoxy-2-((3-methyl-4-(1-methylpiperidin-4-yl)phenyl)amino)pyrimidine-5-carboxamide

To a stirred solution of N-(3-((tert-butyldimethylsilyl)oxy)-2,6-dimethylphenyl)-2-chloro-4-methoxypyrimidine-5-carboxamide (60 mg, 0.142 mmol) was added 3-methyl-4-(1-methylpiperidin-4-yl)aniline (34.9 mg, 0.171 mmol) and the reaction mixture was stirred at 80° C. for 40 minutes under microwave irradiation to afford N-(3-((tert-butyldimethylsilyl)oxy)-2,6-dimethylphenyl)-4-methoxy-2-((3-methyl-4-(1-methyl piperidin-4-yl)phenyl)amino)pyrimidine-5-carboxamide (80 mg, 95% yield) as a brown solid. Chemical Formula: C₃₃H₄₇N₅O₃Si, Exact Mass: 589.3, Molecular Weight: 589.9. LCMS (ESI) m/z=590.4 (M+H), t_{R}=1.835 min, 20% (Method-A).

### N-(3-hydroxy-2,6-dimethylphenyl)-4-methoxy-2-((3-methyl-4-(1-methylpiperidin-4-yl)phenyl)amino)pyrimidine-5-carboxamide (Compound 142)

To a stirred solution of N-(3-((tert-butyldimethylsilyl)oxy)-2,6-dimethylphenyl)-4-methoxy-2-((3-methyl-4-(1-methylpiperidin-4-yl) phenyl) amino)pyrimidine-5-carboxamide (80 mg, 0.136 mmol) was added potassium hydrogen fluoride (15.89 mg, 0.203 mmol). The resultant crude residue was purified by prep. HPLC to afford N-(3-hydroxy-2,6-dimethylphenyl)-4-methoxy-2-((3-methyl-4-(1-methylpiperidin-4-yl)phenyl)amino) pyrimidine-5-carboxamide (10 mg, 16% yield) as an off-white solid. ¹H-NMR (400 MHz, DMSO-d₆): δ 9.92 (s, 1H), 9.14 (s, 1H), 9.11 (s, 1H), 8.69 (s, 1H), 7.58-7.56 (m, 2H), 7.15 (d, J=8.40 Hz, 1H), 6.88 (d, J=8.00 Hz, 1H), 6.69 (d, J=8.00 Hz, 1H), 4.10 (s, 3H), 2.99-2.96 (m, 2H), 2.68-2.63 (m, 1H), 2.34-2.30 (m, 6H), 2.20-2.12 (m, 2H), 2.08 (s, 3H), 2.00 (s, 3H), 1.69-1.65 (m, 4H). Chemical Formula: C₂₇H₃₃N₅O₃, Exact Mass: 475.3, Molecular Weight: 475.6. LCMS (ESI) m/z=476.4 (M+H), t_{R}=1.317 min, 99.3% (Method-C), HPLC purity: 99.5%; t_{R}=3.384 min (Method-A).

### Example 52. Synthesis of N-(4-chloropyridin-3-yl)-4-methoxy-2-((3-methyl-4-(1-methylpiperidin-4-yl)phenyl)amino)pyrimidine-5-carboxamide (Compound 139)

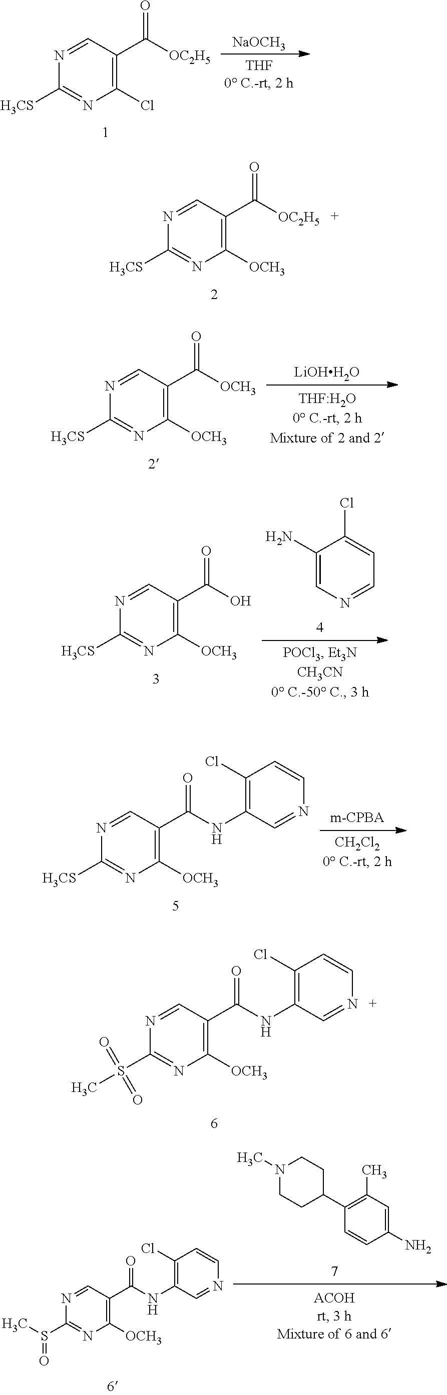

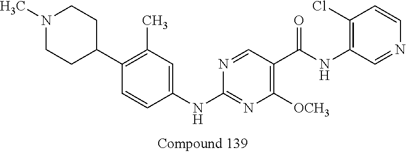 [SMILES: COc1nc(Nc2ccc(C3CCN(C)CC3)c(C)c2)ncc1C(=O)Nc1cnccc1Cl]

### Ethyl 4-methoxy-2-(methylthio)pyrimidine-5-carboxylate and methyl 4-methoxy-2-(methylthio)pyrimidine-5-carboxylate

To a stirred solution of ethyl 4-chloro-2-(methylthio)pyrimidine-5-carboxylate (3 g, 12.89 mmol) in THF (30 mL) was added sodium methoxide (0.697 g, 12.89 mmol) at 0° C. The reaction mixture was stirred at room temperature for 2 h. After completion of the reaction (TLC and UPLC), the reaction mixture was diluted with water and extracted with EtOAc (30 mL×2). The combined organic layer was dried over Na₂SO₄, filtered and concentrated under reduced pressure. The resulting crude material was purified by flash chromatography (SiO₂/100-200 mesh; 0-100% EtOAc/hexanes) to afford ethyl 4-methoxy-2-(methylthio) pyrimidine-5-carboxylate (1 g, 33% yield) as a yellow liquid and methyl 4-methoxy-2-(methylthio)pyrimidine-5-carboxylate (200 mg, 6% yield) as a yellow solid. For ethyl 4-methoxy-2-(methylthio) pyrimidine-5-carboxylate: ¹H-NMR (400 MHz, DMSO-d₆): δ 8.76 (s, 1H), 4.27 (q, J=6.80 Hz, 2H), 4.01 (s, 3H), 2.57 (s, 3H), 1.29 (t, J=7.20 Hz, 3H). Chemical Formula: C₉H₁₂N₂O₃S, Exact Mass: 228.06, Molecular Weight: 228.27. LCMS (ESI) m/z=229.2 (M+H), t_{R}=2.261 min, 97.7% (Method-A). For methyl 4-methoxy-2-(methylthio)pyrimidine-5-carboxylate: ¹H-NMR (400 MHz, DMSO-d₆): δ 8.77 (s, 1H), 4.02 (s, 3H), 3.80 (s, 3H), 2.58 (s, 3H). Chemical Formula: C₈H₁₀N₂O₃S, Exact Mass: 214.04, Molecular Weight: 214.24. LCMS (ESI) m/z=215.2 (M+H), t_{R}=2.038 min, 88.9% (Method-A).

### 4-methoxy-2-(methylthio)pyrimidine-5-carboxylic acid

To a stirred solution of ethyl 4-methoxy-2-(methylthio)pyrimidine-5-carboxylate (1 g, 4.38 mmol) in THE (5 mL) and water (5 mL) was added lithium hydroxide (0.315 g, 13.14 mmol) at 0° C. The reaction mixture was stirred at room temperature for 2 h. After completion of the reaction (TLC and UPLC), the reaction mixture was concentrated under reduced pressure, resultant crude was acidified with dilute HCl and extracted with ethyl acetate (30 mL×2). The combined organic layer was dried over Na₂SO₄, filtered and concentrated under reduced pressure to afford 4-methoxy-2-(methylthio)pyrimidine-5-carboxylic acid (800 mg, 88% yield) as an off white solid. ¹H-NMR (400 MHz, DMSO-d₆): δ 13.09 (s, 1H), 8.75 (s, 1H), 4.00 (s, 3H), 2.57 (s, 3H). Chemical Formula: C₇H₈N₂O₃S, Exact Mass: 200.03, Molecular Weight: 200.21. LCMS (ESI) m/z=201.2 (M+H), t_{R}=1.672 min, 96.1% (Method-A).

### N-(4-chloropyridin-3-yl)-4-methoxy-2-(methylthio)pyrimidine-5-carboxamide

To a stirred solution of 4-methoxy-2-(methylthio)pyrimidine-5-carboxylic acid (0.7 g, 3.50 mmol) in acetonitrile (8 mL) was added POCl₃ (0.489 mL, 5.24 mmol), 4-chloropyridin-3-amine (0.539 g, 4.20 mmol) and Et₃N (1.456 mL, 10.49 mmol) sequentially at 0° C. under nitrogen atmosphere. The reaction mixture was stirred at 50° C. for 3 h. After completion of the reaction (TLC and UPLC), the reaction mixture was concentrated under reduced pressure, resultant crude was basified with aqueous sodium bicarbonate solution and extracted with ethyl acetate (30 mL×2). The combined organic layer was dried over Na₂SO₄, filtered and concentrated under reduced pressure to afford crude. Resultant crude material was washed with Acetonitrile and filtered, solid were dried under reduced pressure to afford N-(4-chloropyridin-3-yl)-4-methoxy-2-(methylthio)pyrimidine-5-carboxamide (0.5 g, 34% yield) as a brown solid. Chemical Formula: C₁₂HnClN₄O₂S, Exact Mass: 310.03, Molecular Weight: 310.76. LCMS (ESI) m/z=311.0 (M+H), t_{R}=2.334 min, 75.3% (Method-A).

### N-(4-chloropyridin-3-yl)-4-methoxy-2-(methylsulfonyl)pyrimidine-5-carboxamide and N-(4-chloropyridin-3-yl)-4-methoxy-2-(methylsulfinyl)pyrimidine-5-carboxamide

To a stirred solution of N-(4-chloropyridin-3-yl)-4-methoxy-2-(methylthio)pyrimidine-5-carboxamide (0.5 g, 1.609 mmol) in CH₂Cl₂ (10 mL) was added m-CPBA (1.157 g, 4.02 mmol) at 0° C. The reaction mixture was stirred at 25° C. for 2 h. After completion of the reaction (TLC and UPLC), the reaction mixture was diluted with 10% Sodium bicarbonate solution and extracted with CH₂Cl₂ (30 mL×2). The combined organic layer was dried over Na₂SO₄, filtered and concentrated under reduced pressure to afford mixture of N-(4-chloropyridin-3-yl)-4-methoxy-2-(methylsulfonyl)pyrimidine-5-carboxamide and N-(4-chloropyridin-3-yl)-4-methoxy-2-(methylsulfinyl)pyrimidine-5-carboxamide (500 mg, crude) as a yellow solid. For N-(4-chloropyridin-3-yl)-4-methoxy-2-(methylsulfonyl)pyrimidine-5-carboxamide: Chemical Formula: C₁₂H₁₁ClN₄O₄S, Exact Mass: 342.02, Molecular Weight: 342.75. LCMS (ESI) m/z=343.2 (M+H), t_{R}=1.734 min, 34% (Method-A). For N-(4-chloropyridin-3-yl)-4-methoxy-2-(methylsulfinyl)pyrimidine-5-carboxamide: Chemical Formula: C₁₂H₁₁ClN₄O₃S, Exact Mass: 326.02, Molecular Weight: 326.75. LCMS (ESI) m/z=327.2 (M+H), t_{R}=1.580 min, 24.8% (Method-A).

### N-(4-chloropyridin-3-yl)-4-methoxy-2-((3-methyl-4-(1-methylpiperidin-4-yl)phenyl)amino)pyrimidine-5-carboxamide (Compound 139)

To a stirred solution N-(4-chloropyridin-3-yl)-4-methoxy-2-(methylsulfonyl)pyrimidine-5-carboxamide (200 mg, 0.584 mmol)(Mixture of 6 and 6′) in Acetic acid (3 mL), was added a 3-methyl-4-(1-methylpiperidin-4-yl)aniline (143 mg, 0.700 mmol) at room temperature. The resulting reaction mixture was stirred at room temperature for 3 h. After completion of the reaction (TLC and UPLC), the reaction mixture was concentrated under reduced pressure. Resultant crude was basified with aqueous sodium bicarbonate solution and extracted with ethyl acetate (30 mL×2). The combined organic layer was dried over Na₂SO₄, filtered and concentrated under reduced pressure. The resultant crude residue was purified by prep. HPLC to get N-(4-chloropyridin-3-yl)-4-methoxy-2-((3-methyl-4-(1-methylpiperidin-4-yl)phenyl)amino)pyrimidine-5-carboxamide (Compound 139, 50 mg, 18% yield) as a yellow solid. ¹H-NMR (400 MHz, DMSO-d₆): δ 10.16 (s, 1H), 9.90 (s, 1H), 9.45 (s, 1H), 8.87 (s, 1H), 8.34 (d, J=5.20 Hz, 1H), 8.21 (s, 1H), 7.69 (d, J=5.20 Hz, 1H), 7.58-7.57 (m, 2H), 7.18 (d, J=9.20 Hz, 1H), 4.20 (s, 3H), 2.97-2.92 (m, 2H), 2.69-2.60 (m, 1H), 2.30 (s, 3H), 2.26 (s, 3H), 2.14-1.97 (m, 2H), 1.70-1.63 (m, 4H). HCOOH Salt. Chemical Formula: C₂₄H₂₇ClN₆O₂, Exact Mass: 466.19, Molecular Weight: 466.97. LCMS (ESI) m/z=467.2 (M+H), t_{R}=1.832 min, 97.6% (Method-A), HPLC purity: 95.6%; t_{R}=3.425 min (Method-A).

### Example 53. Synthesis of N-(2,4-dimethylpyridin-3-yl)-4-methoxy-2-{[3-methyl-4-(1-methylpiperidin-4-yl)phenyl]amino}pyrimidine-5-carboxamide (Compound 112)

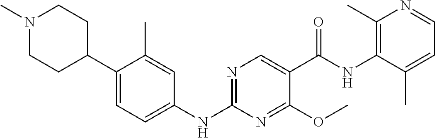 [SMILES: COc1nc(Nc2ccc(C3CCN(C)CC3)c(C)c2)ncc1C(=O)Nc1c(C)ccnc1C]

### Ethyl 4-methoxy-2-(methylsulfanyl)pyrimidine-5-carboxylate

To a solution of ethyl 4-chloro-2-(methylsulfanyl)pyrimidine-5-carboxylate (1.40 g, 6.00 mmol, 1.00 equiv.) in THE (5 ml) was added methanol (267 μl, 6.60 mmol, 1.1 equiv.) followed by potassium carbonate (1.74 g, 12.6 mmol, 2.10 equiv.) and the mixture was heated at 50° C. for 4 days. After cooling to RT the mixture was concentrated under reduced pressure, taken up in DCM (5 ml) and washed with water (3×10 ml). The combined aqueous phases were re-extracted with DCM (3×5 ml) and the combined organic phases were filtered through a phase-separator and concentrated to an oil that solidified upon standing. This crude material was purified by flash chromatography (Biotage Sfar 25 g column) in 0-25% EtOAc/petroleum ether to give the title compound (522 mg, 38%) as a colorless oil. LCMS (ESI+): m/z [M+H]⁺ calcd.: 229, found: 229.

### 4-Methoxy-2-(methylsulfanyl)pyrimidine-5-carboxylic acid

Lithium hydroxide monohydrate (115 mg, 2.75 mmol, 1.20 equiv.) was added to a solution of ethyl 4-methoxy-2-(methylsulfanyl)pyrimidine-5-carboxylate (523 mg, 2.29 mmol, 1.00 equiv.) in THE (2 ml) and water (2 ml) and the mixture was heated at 50° C. After 1.5 h LCMS indicated full conversion of the ethyl ester and the mixture was acidified with HCl (3 ml, 1 M aq.) then concentrated under reduced pressure. The residue was diluted with brine, extracted with ethyl acetate (4×10 ml), the combined organic phases were filtered through a phase-separator and concentrated to give the title compound (443 mg, 95%) as a white solid. ¹H NMR (400 MHz, CDCl₃) δ 8.97 (s, 1H), 4.17 (s, 3H), 2.61 (s, 3H).

### N-(2,4-dimethylpyridin-3-yl)-4-methoxy-2-(methylsulfanyl)pyrimidine-5-carboxamide

Oxalyl chloride (26 μl, 300 μmol, 1.20 equiv.) was added to a stirred solution of 4-methoxy-2-(methylsulfanyl)pyrimidine-5-carboxylic acid (50 mg, 250 μmol, 1.00 equiv.) in dry DCM (2 ml) followed by the addition of a catalytic amount of dry DMF (2 μl). After stirring at room temperature for 2 h, TLC analysis of a sample quenched with MeOH indicated full conversion of the acid starting material, and the solution was concentrated under reduced pressure, followed by concentration from toluene. The residue was dissolved in dry THE (2 ml) followed by the addition of 2,4-dimethylpyridin-3-amine (32 mg, 262 μmol, 1.05 equiv.) and DIPEA (65 μl, 375 mol, 1.50 equiv.) and the resulting solution was stirred at RT for 18 h. After concentration under reduced pressure the crude material was purified by flash chromatography (Biotage Sfar 10 g column) in 1-6% MeOH/DCM containing 0.1% concentrated aqueous ammonia. The fractions containing product were concentrated under reduced pressure to give the title compound (58 mg, 76%) as a white solid. LCMS (ESI+): m/z [M+H]⁺ calcd.: 305, found: 305.

### N-(2,4-dimethylpyridin-3-yl)-4-methoxy-2-[[3-methyl-4-(1-methylpiperidin-4-yl)phenyl]amino}pyrimidine-5-carboxamide (Compound 112)

mCPBA (44 mg, 75%, 190 μmol, 1.0 equiv.) was added to a stirred solution of N-(2,4-dimethylpyridin-3-yl)-4-methoxy-2-(methylsulfanyl)pyrimidine-5-carboxamide (56 mg, 190 μmol, 1.0 equiv.) in DCM (5 ml) at 0° C. Upon full conversion of the thioether starting material as indicated by LCMS the mixture was concentrated under reduced pressure. The residue was dissolved in acetonitrile (2 ml) followed by the addition of methanesulfonic acid (25 μl, 385 μmol, 2.0 equiv.) and 3-methyl-4-(1-methylpiperidin-4-yl)aniline (39 mg, 190 mol, 1.0 equiv.). After stirring at 60° C. for 2 h the mixture was concentrated, and the residue was purified by prep-LCMS running a focused gradient of acetonitrile in 0.1% NH₃/water on a Kromasil 100-5-C18 250×30 mm LC-column. Collected fractions were combined and lyophilized to give the title compound as a white solid (22.4 mg, 26%). ¹H NMR (400 MHz, DMSO-d₆) δ 9.97 (s, 1H), 9.37 (s, 1H), 8.71 (s, 1H), 8.24 (d, J=4.7 Hz, 1H), 7.66-7.52 (m, 2H), 7.23-7.11 (m, 2H), 4.10 (s, 3H), 2.90 (d, J=10.4 Hz, 2H), 2.69-2.57 (m, 1H), 2.38 (s, 3H), 2.29 (s, 3H), 2.22 (s, 3H), 2.20 (s, 3H), 2.10-1.96 (m, 2H), 1.70-1.58 (m, 4H). LCMS (ESI+): m/z [M+H]⁺ calcd.: 461, found: 461. HPLC: 98%; t_{R}: 5.69 min (Method-C)

### Example 54. Synthesis of N-(2-chloro-4-methylpyridin-3-yl)-4-methoxy-2-{[3-methyl-4-(1-methylpiperidin-4-yl)phenyl]amino}pyrimidine-5-carboxamide (Compound 117)

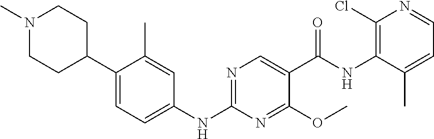 [SMILES: COc1nc(Nc2ccc(C3CCN(C)CC3)c(C)c2)ncc1C(=O)Nc1c(C)ccnc1Cl]

### N-(2-chloro-4-methylpyridin-3-yl)-4-methoxy-2-(methylsulfanyl)pyrimidine-5-carboxamide

The title compound was prepared in a similar way as ethyl 4-methoxy-2-(methylsulfanyl)pyrimidine-5-carboxamide in the previous Example using 2-chloro-4-methylpyridin-3-amine (37 mg, 260 μmol, 1.0 equiv.) and ethyl 4-methoxy-2-(methylsulfanyl)pyrimidine-5-carboxylate. Additional purification by reverse phase chromatography (Gemini NX—C18, 21*150 mm, water (0.1% formic acid)/acetonitrile, 30-60% gradient over 15 minutes, 20 ml/min) followed by lyophilization of the fractions containing product gave the title compound (41 mg, 51%) as a white solid. LCMS (ESI+): m/z [M+H]⁺ calcd.: 325, found: 325.

### N-(2-chloro-4-methylpyridin-3-yl)-4-methoxy-2-{[3-methyl-4-(1-methylpiperidin-4-yl)phenyl]amino}pyrimidine-5-carboxamide (Compound 117)

The title compound was prepared in a similar way as Compound 112 in the previous Example using N-(2-chloro-4-methylpyridin-3-yl)-4-methoxy-2-(methylsulfanyl)pyrimidine-5-carboxamide (41 mg, 126 μmol). The crude product was purified by reverse phase chromatography (Gemini NX—C18, 21*150 mm, water (0.1% TFA)/acetonitrile, 5-30% gradient over 12 minutes, 25 ml/min). The product-containing fractions were lyophilized to give the title compound (30 mg, 34%) as a white solid. ¹H NMR (400 MHz, DMSO-d₆) δ 10.09 (s, 1H), 9.54 (s, 1H), 9.38 (d, J=11.0 Hz, 1H), 8.75 (s, 1H), 8.23 (d, J=4.9 Hz, 1H), 7.60 (dd, J=11.6, 3.3 Hz, 2H), 7.40 (d, J=5.0 Hz, 1H), 7.10 (d, J=8.4 Hz, 1H), 4.11 (s, 3H), 3.52 (d, J=11.9 Hz, 2H), 3.19-3.07 (m, 2H), 2.95 (tq, J=7.2, 3.6 Hz, 1H), 2.82 (d, J=4.6 Hz, 3H), 2.32 (s, 3H), 2.27 (s, 3H), 1.96-1.69 (m, 4H). LCMS (ESI+): m/z [M+H]⁺ calcd.: 481, found: 481. HPLC: 97%; t_{R}: 7.54 min (Method-C)

### Example 55. Synthesis of N-(2,6-dichlorophenyl)-2-{[4-(4-methylpiperazin-1-yl)phenyl]amino}-4-(2,2,2-trifluoroethoxy)pyrimidine-5-carboxamide (Compound 103)

### N-(2,6-dichlorophenyl)-2-(methylsulfanyl)-4-(2,2,2-trifluoroethoxy)pyrimidine-5-carboxamide

A mixture of 4-chloro-N-(2,6-dichlorophenyl)-2-(methylsulfanyl)pyrimidine-5-carboxamide (100 mg, 287 μmol, 1.00 equiv.) and potassium carbonate (40 mg, 290 mol, 1.01 equiv.) and 2,2,2-trifluoroethanol (1 ml, excess) was heated at 80° C. for 15 min then diluted with water (30 ml). A white precipitate was collected by filtration and dried in vacuo to yield the title compound (103 mg, 87%) as a white solid which was used in the following step without purification. LCMS (ESI+): m/z [M+H]⁺ calcd.: 412, found: 412.

### N-(2,6-dichlorophenyl)-2-[[4-(4-methylpiperazin-1-yl)phenyl]amino}-4-(2,2,2-trifluoroethoxy)pyrimidine-5-carboxamide (Compound 103)

mCPBA (58 mg, 75%, 250 μmol, 1.0 equiv.) was added to a stirred solution of N-(2,6-dichlorophenyl)-2-(methylsulfanyl)-4-(2,2,2-trifluoroethoxy)pyrimidine-5-carboxamide (103 mg, 250 μmol, 1.0 equiv.) in DCM (5 ml) at room temperature. After 15 min, LCMS indicated full conversion to the corresponding sulfoxide and the mixture was concentrated under reduced pressure. The residue was dissolved in acetonitrile (6 ml) and ⅓ of this solution (2 ml, 83 μmol intermediate) was treated with methanesulfonic acid (11 μl, 170 μmol, 2.0 equiv.) followed by 4-(4-methylpiperazin-1-yl)aniline (16 mg, 84 mol, 1.0 equiv.) and the resulting mixture was heated at 60° C. for 2 h. The reaction mixture was concentrated under reduced pressure, redissolved in dioxane/water, acidified with TFA and purified by reversed phase chromatography (Gemini NX—C18, 21*150 mm, water (0.1% TFA)/acetonitrile, 20-50% gradient over 12 minutes, 25 ml/min). The product-containing fractions were loaded on an SCX-2 column (1 g, 0.61 mmol loading capacity), washed with methanol, eluted with ammonia (1.4 M in methanol), and concentrated under reduced pressure to give the title compound (17 mg, 37%) as a white solid. ¹H NMR (400 MHz, DMSO-d₆) δ 10.07 (s, 1H), 9.55 (s, 1H), 8.70 (s, 1H), 7.59 (d, J=8.1 Hz, 2H), 7.53 (d, J=8.6 Hz, 2H), 7.39 (t, J=8.1 Hz, 1H), 6.93 (d, J=8.7 Hz, 2H), 5.17 (q, J=8.9 Hz, 2H), 3.17-3.04 (m, 4H), 2.47 (d, J=5.6 Hz, 4H), 2.23 (s, 3H). LCMS (ESI+): m/z [M+H]⁺ calcd.: 555, found: 555. HPLC: 98%; t_{R}: 9.27 min (Method-C)

### Example 56. Synthesis of N-(2,6-dichlorophenyl)-2-{[3-methyl-4-(1-methylpiperidin-4-yl)phenyl]amino}-4-(2,2,2-trifluoroethoxy)pyrimidine-5-carboxamide (Compound 104)

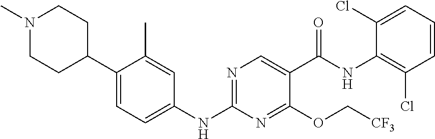 [SMILES: Cc1cc(Nc2ncc(C(=O)Nc3c(Cl)cccc3Cl)c(OCC(F)(F)F)n2)ccc1C1CCN(C)CC1]

The title compound was prepared in a similar way as Compound 103, above, using 3-methyl-4-(1-methylpiperidin-4-yl)aniline (17 mg, 83 μmol, 1.00 equiv.) to give the title compound (18 mg, 38%) as a white solid. ¹H NMR (400 MHz, DMSO-d₆) δ 10.15 (s, 1H), 9.63 (s, 1H), 8.73 (s, 1H), 7.59 (d, J=8.1 Hz, 2H), 7.52 (s, 1H), 7.50-7.43 (m, 1H), 7.40 (t, J=8.1 Hz, 1H), 7.17 (d, J=8.5 Hz, 1H), 5.20 (q, J=8.9 Hz, 2H), 2.89 (d, J=10.8 Hz, 2H), 2.60 (q, J=7.1 Hz, 1H), 2.29 (s, 3H), 2.22 (s, 3H), 2.09-1.93 (m, 2H), 1.64 (h, J=3.5 Hz, 4H). LCMS (ESI+): m/z [M+H]⁺ calcd.: 568, found: 568. HPLC: 96%; t_{R}: 9.79 min (Method-C).

### Example 57. Synthesis of N-2,6-dichlorophenyl-4-methoxy-2-[6-(1-methyl-4-piperidyl)-3-pyridylamino]-5-pyrimidinecarboxamide (Compound 183)

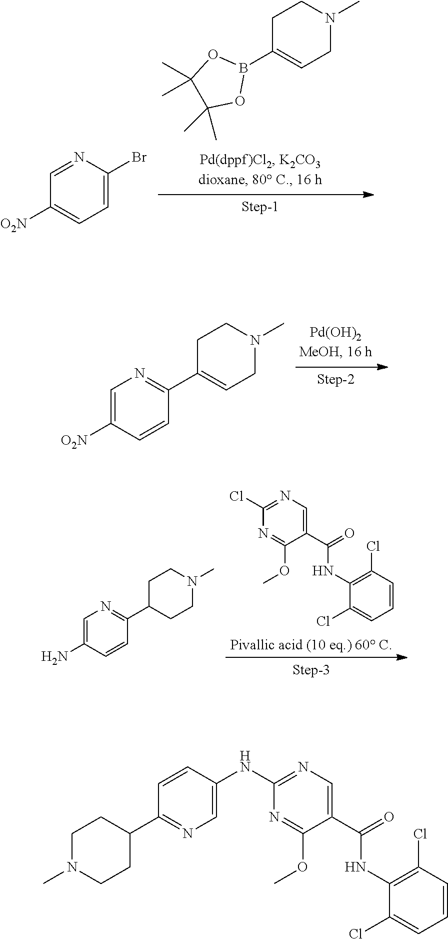

### 1′-methyl-5-nitro-1′,2′,3,6′-tetrahydro-2,4′-bipyridine

To a stirred solution of 2-bromo-5-nitropyridine (1 g, 4.93 mmol) in 1,4-dioxane (24 ml): water (16 ml), K₂CO₃ (0.887 g, 14.78 mmol) and 1-methyl-4-(4,4,5,5-tetramethyl-1,3,2-dioxaborolan-2-yl)-1,2,3,6-tetrahydropyridine (1.099 g, 4.93 mmol) were added and then bubbled with nitrogen for 15 min. To the reaction mixture, Pd(dppf)Cl₂ (0.180 g, 0.246 mmol) was added and then the mixture was stirred under microwave irradiation at 90° C. for 4 h. The progress of the reaction was monitored by TLC and LCMS. After the completion of the reaction, the reaction mixture was diluted with water (100 mL) and extracted with 10% methanol-DCM (250 mL×3). The combined organic extract was washed with brine (150 mL), dried over anhydrous sodium sulfate, filtered, and concentrated under reduced pressure (bath temperature: 45° C.) to afford crude product. The crude product was purified by combi flash column chromatography (silica-gel, mesh size-60-120) using Methanol-DCM (5-10%) as an eluent to obtain 1′-methyl-5-nitro-1′,2′,3′,6′-tetrahydro-2,4′-bipyridine (300 mg, 1.122 mmol, 22.78% yield) as a brown viscous liquid. LCMS (ESI) m/z=220.1 (M+H), t_{R}. 0.91 min, 81.80% (Method D).

### 6-(1-methylpiperidin-4-yl)pyridin-3-amine

To a stirred solution of 1′-methyl-5-nitro-1′,2′,3′,6′-tetrahydro-2,4′-bipyridine (300 mg, 1.368 mmol) in MeOH (20 ml), Pd(OH)₂ (189 mg, 1.779 mmol), were added at room temperature portion-wise in the reaction mixture. The reaction mixture was stirred at room temperature for 16 h under a hydrogen atmosphere. The progress of the reaction was monitored by TLC and LCMS after the completion of the reaction. The reaction mixture was filtered through a celite bed, and the bed was washed with Methanol (500 mL). Filtrate was concentrated under reduced pressure (bath temperature: 45° C.) to afford crude product. The crude compound was not purified but directly taken for next step.

### N-(2,6-dichlorophenyl)-4-methoxy-2-((6-(1-methylpiperidin-4-yl)pyridin-3-yl)amino)pyrimidine-5-carboxamide

To a stirred solution of 2-chloro-N-(2,6-dichlorophenyl)-4-methoxypyrimidine-5-carboxamide (319 mg, 0.959 mmol) in pivalic acid (980 mg, 9.59 mmol), 6-(1-methylpiperidin-4-yl)pyridin-3-amine (220 mg, 1.151 mmol) was added at room temperature. The reaction mixture was stirred at 60° C. for 12 h. The progress of the reaction was monitored by LCMS after the completion of the reaction. The reaction mixture was diluted with ice water and extracted with 10% methanol-DCM. The organic layer was washed with 5% sodium hydroxide solution, water, and dried over anhydrous sodium sulfate and concentrated under reduced pressure to afford crude product which was purified by preparative HPLC to afford N-(2,6-dichlorophenyl)-4-methoxy-2-((6-(1-methylpiperidin-4-yl)pyridin-3-yl)amino)pyrimidine-5-carboxamide (18 mg). LCMS (ESI) m/z=487.0 & 489.0 (M+H), t_{R}=1.28 min, 92.49% (Method-A). 1H-NMR (400 MHz, DMSO-do): δ 10.23 (s, 1H), 9.60 (s, 1H), 8.84 (d, J=2.00 Hz, 1H), 8.77 (s, 1H), 8.12 (dd, J=2.80, 8.60 Hz, 1H), 7.59-7.56 (m, 2H), 7.39 (t, J=8.00 Hz, 1H), 7.26 (d, J=8.40 Hz, 1H), 4.11 (s, 3H), 2.89-2.86 (m, 2H), 2.68 (m, 1H), 2.20 (s, 3H), 2.01-1.91 (m, 2H), 1.76-1.69 (m, 4H).

### Example 58. Synthesis of N-2,6-dichlorophenyl-2-{1-[(R)-4-azepanyl]-4-pyrazolylamino}-4-methoxy-5-pyrimidinecarboxamide (Compound 186) and N-2,6-dichlorophenyl-2-{1-[(S)-4-azepanyl]-4-pyrazolylamino}-4-methoxy-5-pyrimidinecarboxamide (Compound 187)

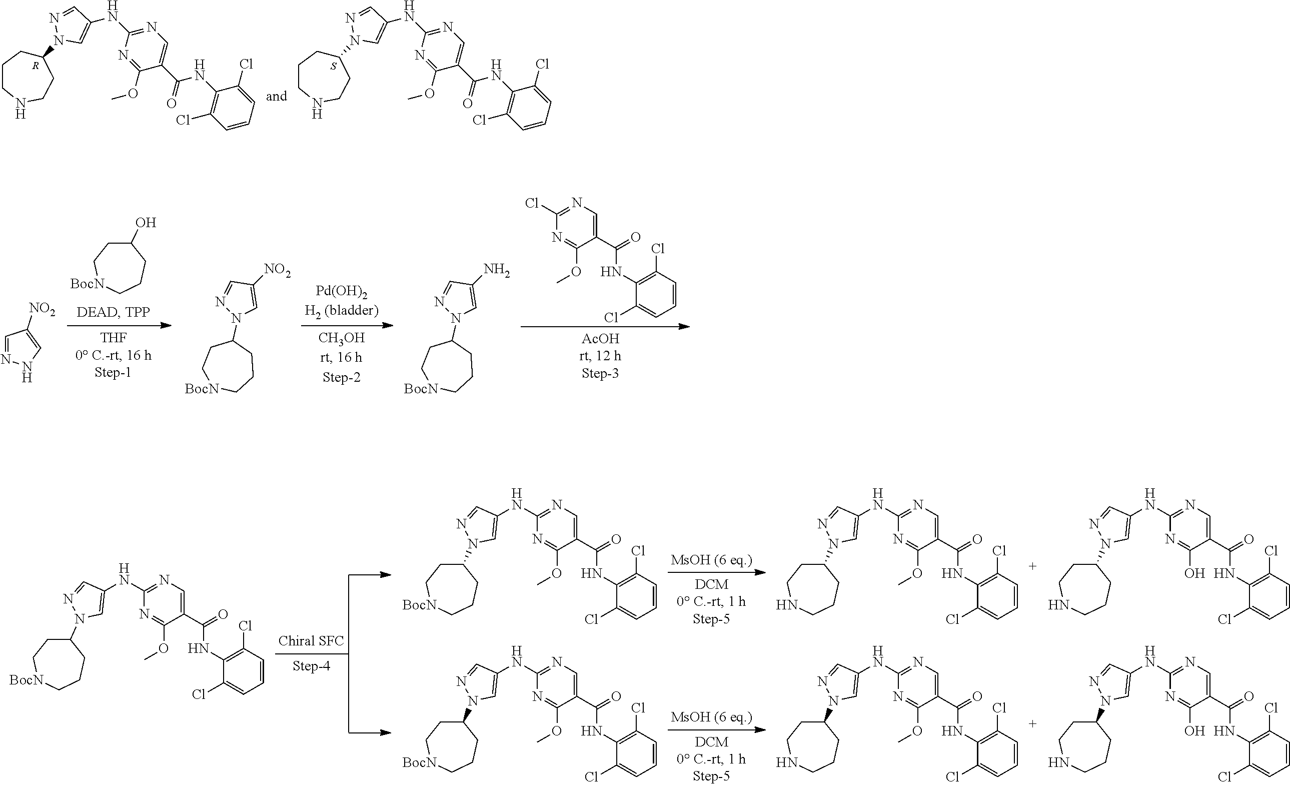

### tert-butyl 4-(4-nitro-1H-pyrazol-1-yl)azepane-1-carboxylate

To a stirred solution of tert-butyl 4-hydroxyazepane-1-carboxylate (1 g, 4.64 mmol) and 4-nitro-1H-pyrazole (0.525 g, 4.64 mmol) in tetrahydrofuran (20 ml) was added triphenylphosphine (4.87 g, 18.58 mmol) at room temperature, followed by diisopropyl azodicarboxylate (1.127 ml, 5.75 mmol) under inert atmosphere at 0° C. The resulting reaction mixture was stirred at 25° C. for 16 h. The progress of reaction was monitored by TLC and UPLC., the reaction mixture was concentrated under reduced pressure. The resulting crude material was purified by flash column chromatography (SiO₂/100-200 mesh; ~20-30% EtOAc/Hexane) to afford tert-butyl 4-(4-nitro-1H-pyrazol-1-yl)azepane-1-carboxylate (1.2 g, 2.90 mmol, 62.4% yield) as a colorless gummy solid. LCMS (ESI) m/z=211.2 [M−100], t_{R}=1.90 min, 75.4% (Method A).

### tert-butyl 4-(4-amino-1H-pyrazol-1-yl)azepane-1-carboxylate (4)

To a degassed solution of tert-butyl 4-(4-nitro-1H-pyrazol-1-yl)azepane-1-carboxylate (0.5 g, 1.611 mmol) in methanol (20 mL) was added 10% palladium hydroxide on carbon (50 mg) under nitrogen atmosphere. The reaction mixture was stirred under a hydrogen atmosphere using hydrogen bladder pressure at room temperature for 16 h. The progress of the reaction was monitored by TLC and UPLC., the reaction mixture was filtered under a nitrogen atmosphere through a pad of celite to remove the catalyst, and the celite pad was washed with methanol (50 mL). The filtrate was concentrated under reduced pressure to afford tert-butyl 4-(4-amino-1H-pyrazol-1-yl)azepane-1-carboxylate (0.43 g, 1.196 mmol, 74.3% yield). LCMS (ESI) m/z=281.4 [M+H], t_{R}=1.02 min, 78.51% (Method A).

### tert-butyl 4-(4-((5-((2,6-dichlorophenyl)carbamoyl)-4-methoxypyrimidin-2-yl)amino)-1H-pyrazol-1-yl)azepane-1-carboxylate

To a stirred solution of 2-chloro-N-(2,6-dichlorophenyl)-4-methoxypyrimidine-5-carboxamide (463 mg, 1.391 mmol) in acetic acid (5 mL) was added tert-butyl 4-(4-amino-1H-pyrazol-1-yl)azepane-1-carboxylate (325 mg, 1.159 mmol) at room temperature. The resulting reaction mixture was stirred for 12 h at room temperature. The progress of the reaction was monitored by TLC., the reaction mixture was diluted with water, washed with aqueous NaHCO₃, and extracted with 10% methanol-DCM (20 mL×2). The combined organic layer was dried over Na₂SO₄, filtered, and concentrated under reduced pressure. The resulting crude material was purified by flash chromatography (SiO₂/100-200 mesh; 0-10% CH₃OH/CH₂Cl₂) to afford tert-butyl 4-(4-((5-((2,6-dichlorophenyl)carbamoyl)-4-methoxypyrimidin-2-yl)amino)-1H-pyrazol-1-yl)azepane-1-carboxylate (0.32 g, 0.239 mmol, 48% yield) as an off white solid. LCMS (ESI) m/z=577.8 [M+2], t_{R}=2.048 min, 99.51% (Method-A).

### Chiral SFC separation of tert-butyl 4-(4-((5-((2,6-dichlorophenyl)carbamoyl)-4-methoxypyrimidin-2-yl)amino)-1H-pyrazol-1-yl)azepane-1-carboxylate

The racemic compound (0.32 g, 0.239 mmol), was separated via the chiral SFC purification (Lux I-Amylose-3_0.5% IPAm in MeOH_ACN_35_0_3.lcd). Pure fractions were collected and lyophilized to afford tert-butyl (R)-4-(4-((5-((2,6-dichlorophenyl)carbamoyl)-4-methoxypyrimidin-2-yl)amino)-1H-pyrazol-1-yl)azepane-1-carboxylate (70 mg, 0.121 mmol, 26.8% yield), LCMS (ESI) m/z=577.5 [M+2], t_{R}=2.069 min, 99.827% (Method-A) and tert-butyl (S)-4-(4-((5-((2,6-dichlorophenyl)carbamoyl)-4-methoxypyrimidin-2-yl)amino)-1H-pyrazol-1-yl)azepane-1-carboxylate (100 mg, 0.172 mmol, 38.1% yield). LCMS (ESI) m/z=577.4 [M+2], t_{R}=2.06 min, 98.44% (Method-A).

### (S)-2-((1-(azepan-4-yl)-1H-pyrazol-4-yl)amino)-N-(2,6-dichlorophenyl)-4-methoxypyrimidine-5-carboxamide

To a screw caped reaction vial with magnetic bar was charged tert-butyl (R)-4-(4-((5-((2,6-dichlorophenyl)carbamoyl)-4-methoxypyrimidin-2-yl)amino)-1H-pyrazol-1-yl)azepane-1-carboxylate (100 mg, 0.173 mmol) in DCM (5 mL) and methanesulfonic acid (0.034 ml, 0.520 mmol) was added dropwise at 0° C. and continued to stir at 0° C. for 1 h. Progress of the reaction was monitored by TLC and LCMS., the reaction mixture was quenched/diluted with an aqueous NaHCO₃ solution and extracted with 10% MeOH/DCM (10 mL×2). The combined organic layer was concentrated under reduced pressure to get crude compound, which were further purified by reverse-phase column chromatography to afford (R)-2-((1-(azepan-4-yl)-1H-pyrazol-4-yl)amino)-N-(2,6-dichlorophenyl)-4-methoxypyrimidine-5-carboxamide (41 mg, 0.069 mmol, 43.6% yield) as an off white solid. LCMS (ESI) m/z=476.0 [M+H], t_{R}=1.307 min, 97.73% (Method-D). ¹H-NMR (400 MHz, DMSO-d₆): δ 10.15 (s, 1H), 9.50 (s, 1H), 8.73 (d, J=21.20 Hz, 1H), 7.62-7.49 (m, 3H), 7.39 (t, J=8.40 Hz, 1H), 4.46 (d, J=2814.80 Hz, 1H), 4.45 (s, 1H), (d, J=39.20 Hz, 3H), 2.79-2.78 (m, 4H), 2.06-1.91 (m, 4H), 1.76 (s, 1H), 1.66-1.60 (m, 1H).

### (R)-2-((1-(azepan-4-yl)-1H-pyrazol-4-yl)amino)-N-(2,6-dichlorophenyl)-4-methoxypyrimidine-5-carboxamide

To a screw caped reaction vial with a magnetic bar was charged tert-butyl (S)-4-(4-((5-((2,6-dichlorophenyl)carbamoyl)-4-methoxypyrimidin-2-yl)amino)-1H-pyrazol-1-yl)azepane-1-carboxylate (100 mg, 0.173 mmol) in DCM (5 mL), and methanesulfonic acid (0.034 ml, 0.520 mmol) was added dropwise at 0° C. and continued to stir at 0° C. for 1 h. Progress of reactions was monitored by TLC and LCMS., the reaction mixture was quenched/diluted with aqueous NaHCO₃ solution and extracted with 10% MeOH/DCM (10 mL×2). The combined organic layer was concentrated under reduced pressure to get crude compound, which was further purified by reverse-phase column chromatography to afford (S)-2-((1-(azepan-4-yl)-1H-pyrazol-4-yl)amino)-N-(2,6-dichlorophenyl)-4-methoxypyrimidine-5-carboxamide (63.7 mg, 0.114 mmol, 73.8% yield) as an off white solid. LCMS (ESI) m/z=476.0 [M+H], t_{R}=1.296 min, 97.74% (Method-D). ¹H-NMR (400 MHz, DMSO-d₆, at 80° C.): δ 9.79 (s, 1H), 9.34 (s, 1H), 8.73 (s, 1H), 7.93 (s, 1H), 7.61 (s, 1H), 7.55 (d, J=8.40 Hz, 2H), 7.37 (t, J=8.40 Hz, 1H), 4.45-4.40 (m, 1H), 4.14 (s, 3H), 2.97-2.87 (m, 2H), 2.84-2.80 (m, 2H), 1.66-1.59 (m, 1H).

### Example 59. Synthesis of N-2,6-dichlorophenyl-2-{1-[(R)-1-methyl-4-azepanyl]-4-pyrazolylamino}-4-methoxy-5-pyrimidinecarboxamide (Compound 188) and N-2,6-dichlorophenyl-2-{1-[(S)-1-methyl-4-azepanyl]-4-pyrazolylamino}-4-methoxy-5-pyrimidinecarboxamide (Compound 189)

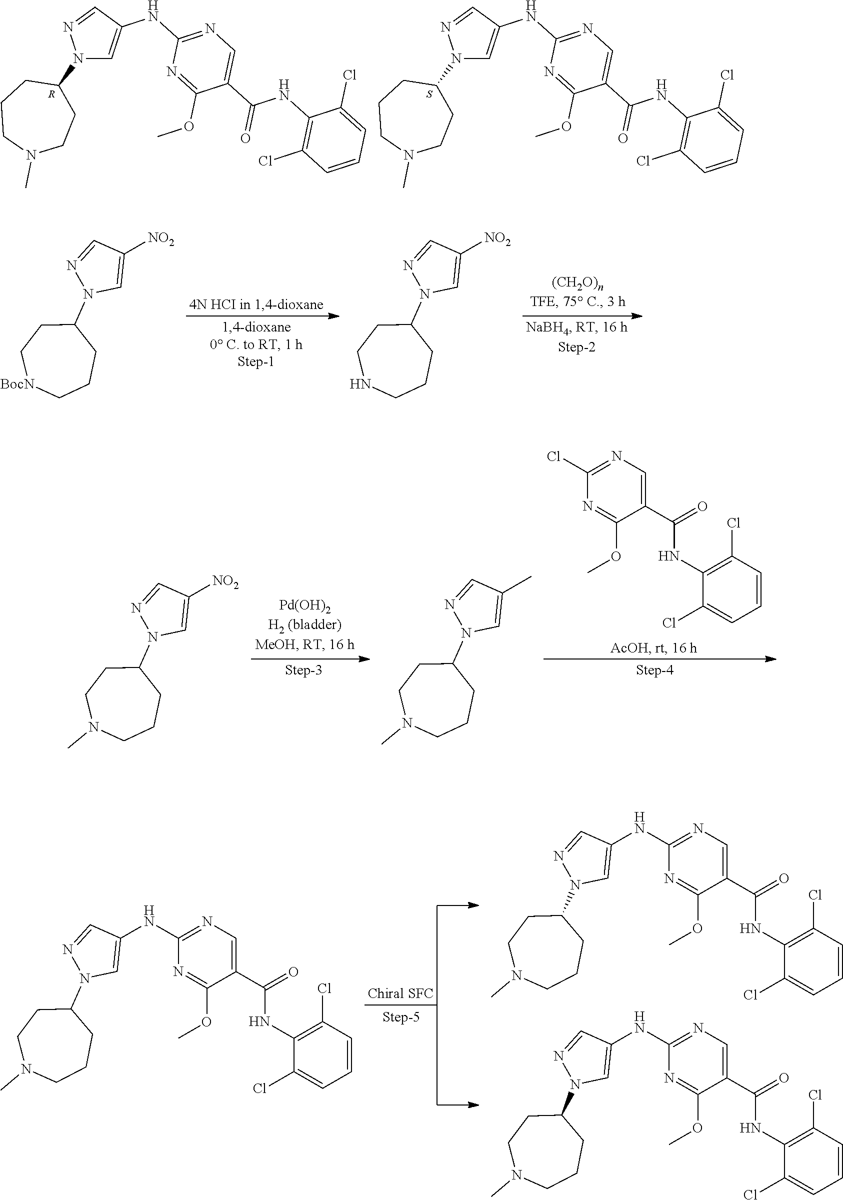

### 4-(4-nitro-1H-pyrazol-1-yl)azepane

To a stirred solution of tert-butyl 4-(4-nitro-1H-pyrazol-1-yl)azepane-1-carboxylate (1 g, 3.22 mmol) in DCM (20 ml) at 0° C. was added 4M HCl (9.67 ml, 38.7 mmol) dropwise at 0° C. The reaction mixture was allowed to stir at 25° C. for 2 h. The progress of the reaction was monitored by TLC and UPLC., the reaction mixture was concentrated under reduced pressure. The crude compound was washed with saturated sodium bicarbonate (50 mL) and extracted with 100 mL of 10% methanol in DCM solution. The organic layer was washed with brine (150 mL), dried over anhydrous sodium sulfate, filtered, and concentrated under reduced pressure (bath temperature: 42° C.) to afford 4-(4-nitro-1H-pyrazol-1-yl)azepane (600 mg, 2.80 mmol, 87% yield) as a brown viscous liquid.

### 1-methyl-4-(4-nitro-1H-pyrazol-1-yl)azepane

To a stirred solution of 4-(4-nitro-1H-pyrazol-1-yl)azepane (1 g, 4.76 mmol) in TFE (10 mL) at 0° C., was added paraformaldehyde (0.714 g, 23.78 mmol) and the reaction mixture stirred at 75° C. for 2 h. The progress of the reaction was monitored by TLC. The reaction mixture was cooled at 0° C., and sodium borohydride (0.414 g, 10.94 mmol) was added portion-wise. The reaction mixture was allowed to be stirred at 75° C. for 16 h. The progress of the reaction was monitored by TLC and UPLC., the reaction mixture was diluted with water (150 mL) and extracted with 10% methanol: DCM (50 mL×2). The combined organic layer was dried over anhydrous sodium sulphate, filtered, and concentrated under reduced pressure (bath temperature: 45° C.) to afford crude product.

### 1-(1-methylazepan-4-yl)-1H-pyrazol-4-amine

To a degassed solution of 1-methyl-4-(4-nitro-1H-pyrazol-1-yl)azepane (1.120 g, 4.99 mmol) in methanol (20 mL) was added 10% palladium hydroxide on carbon (150 mg) under inert atmosphere. The reaction mixture was stirred under a hydrogen atmosphere using hydrogen bladder pressure at room temperature for 16 h. The progress of the reaction was monitored by TLC and UPLC., the reaction mixture was filtered under a nitrogen atmosphere through a pad of celite to remove the catalyst, and the celite pad was washed with methanol (50 mL). The filtrate was concentrated under reduced pressure to afford 1-(1-methylazepan-4-yl)-1H-pyrazol-4-amine (0.873 g, 4.40 mmol, 88% yield). Note: Crude product not isolated, taken as such for subsequent step. LCMS (ESI) m/z=195.1 [M+H], t_{R}=0.37 min, 98.29% (Method D).

### N-(2,6-dichlorophenyl)-4-methoxy-2-((1-(1-methylazepan-4-yl)-1H-pyrazol-4-yl)amino)pyrimidine-5-carboxamide

To a stirred solution of 2-chloro-N-(2,6-dichlorophenyl)-4-methoxypyrimidine-5-carboxamide (250 mg, 0.752 mmol) in acetic acid (5 mL) was added 1-(1-methylazepan-4-yl)-1H-pyrazol-4-amine (250 mg, 0.752 mmol) at room temperature. The resulting reaction mixture was stirred for 12 h at room temperature. The progress of the reaction was monitored by TLC., the reaction mixture was diluted with water, washed with aqueous NaHCO₃. and extracted with 10% Methanol-DCM (30 mL×2). The combined organic layer was dried over Na₂SO₄, filtered, and concentrated under reduced pressure. The resulting crude material was purified by flash chromatography (SiO₂/100-200 mesh; 0-10% CH₃OH/CH₂Cl₂) to afford N-(2,6-dichlorophenyl)-4-methoxy-2-((1-(1-methylazepan-4-yl)-1H-pyrazol-4-yl)amino)pyrimidine-5-carboxamide (51.5 mg, 0.102 mmol, 13.55% yield) as an off white solid. LCMS (ESI) m/z=491.4 [M+1], t_{R}=1.227 min, 97.4% (Method-A).

### Chiral SFC separation of tert-butyl 4-(4-((5-((2,6-dichlorophenyl)carbamoyl)-4-methoxypyrimidin-2-yl)amino)-1H-pyrazol-1-yl)azepane-1-carboxylate

The racemic compound, (0.32 g, 0.239 mmol) was separated via chiral SFC purification. Using the method CHIRALPAK-IK_0.5% IPAm in IPA. Pure fractions were collected and lyophilized to afford (R)—N-(2,6-dichlorophenyl)-4-methoxy-2-((1-(1-methylazepan-4-yl)-1H-pyrazol-4-yl)amino)pyrimidine-5-carboxamide (11 mg, 0.021 mmol, 20.06% yield) and (S)—N-(2,6-dichlorophenyl)-4-methoxy-2-((1-(1-methylazepan-4-yl)-1H-pyrazol-4-yl)amino)pyrimidine-5-carboxamide (11 mg, 0.022 mmol, 21.14% yield) as off-white solids.

Analytical data of R)—N-(2,6-dichlorophenyl)-4-methoxy-2-((1-(1-methylazepan-4-yl)-1H-pyrazol-4-yl)amino)pyrimidine-5-carboxamide: LCMS (ESI) m/z=490.2 [M+H], t_{R}=1.29 min, 92.94% (Method-D). ¹H-NMR (400 MHz, DMSO-d6): δ 10.14 (s, 1H), 9.50 (s, 1H), 8.73 (s, 1H), 7.96 (s, 1H), 7.62-7.49 (m, 3H), 7.42-7.26 (m, 1H), 4.47 (s, 1H), 2.69 (d, J=12.80 Hz, 1H), 2.58-2.51 (m, 3H), 2.34-2.30 (m, 3H), 2.11-2.03 (m, 4H), 1.80 (dd, J=4.00, 9.80 Hz, 1H), 1.65 (t, J=153.60 Hz, 1H).

Analytical data of (S)—N-(2,6-dichlorophenyl)-4-methoxy-2-((1-(1-methylazepan-4-yl)-1H-pyrazol-4-yl)amino)pyrimidine-5-carboxamide: LCMS (ESI) m/z=490.0 [M+H], t_{R}=1.37 min, 97.68% (Method-D). ¹H-NMR (400 MHz, DMSO-d6): δ 10.14 (s, 1H), 9.50 (s, 1H), 8.73 (s, 1H), 7.96 (s, 1H), 7.62-7.57 (m, 3H), 7.38 (t, J=8.00 Hz, 1H), 4.47 (s, 1H), 2.68 (t, J=2.00 Hz, 1H), 2.56-2.51 (m, 3H), 2.34-2.29 (m, 3H), 2.10-2.02 (m, 4H), 1.82-1.77 (m, 1H), 1.69-1.62 (m, 1H).

### Example 60. Synthesis of N-(2-bromo-6-chlorophenyl)-4-methoxy-2-[4-(3-pyrrolidinyloxy)-3-toluidino]-5-pyrimidinecarboxamide (Compound 190)

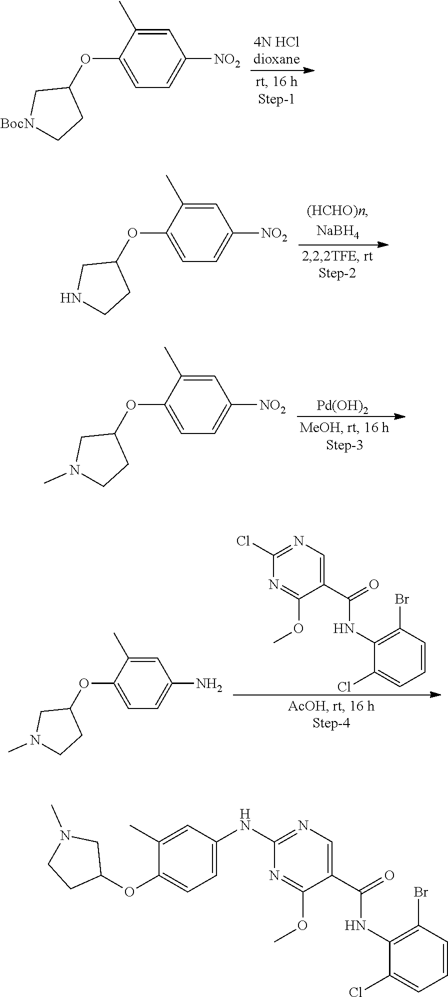

### 3-(2-methyl-4-nitrophenoxy)pyrrolidine

To a stirred solution of tert-butyl 3-(2-methyl-4-nitrophenoxy)pyrrolidine-1-carboxylate (2 g, 6.20 mmol) in dioxane (5 mL), was added 4M HCl in dioxane (0.188 ml, 6.20 mmol)) at 0° C. under inert atmosphere. The resulting reaction mixture was stirred at room temperature for 3 h. The progress of the reaction was monitored by TLC and LCMS., the reaction mixture was concentrated under reduced pressure. The resulting crude residue was washed with sodium bicarbonate and extracted with DCM (2×200 mL) to get the crude 3-(2-methyl-4-nitrophenoxy)pyrrolidine (1.2 g, 4.91 mmol, 79% yield) as a gummy solid, which was taken into the next step without any further purification. LCMS (ESI) m/z=223.1 (M+H), t_{R}: 1.076, 90.74% (Method-A)

### 1-methyl-3-(2-methyl-4-nitrophenoxy)pyrrolidine

To a stirred solution of 3-(4-nitrophenoxy)pyrrolidine (500 mg, 2.401 mmol) in 2,2,2-trifluoroethanol was added paraformaldehyde (361 mg, 12.01 mmol) at 0° C. under inert atmosphere. The resulting reaction mixture was stirred at 70° C. temperature for 6 h. The reaction was then cooled to 0° C. and sodium borohydride (273 mg, 7.20 mmol) was added lotwise. The resulting reaction mixture was stirred at room temperature for 16 h. The progress of the reaction was monitored by TLC and LCMS., the reaction mixture was concentrated under reduced pressure. Water was added to the resulting crude residue and extracted with DCM to get the crude 1-methyl-3-(2-methyl-4-nitrophenoxy)pyrrolidine (300 mg, 1.270 mmol, 52.9% yield) as a yellow semi-solid, which was taken into the next step without any further purification. LCMS (ESI) m/z=237.0 (M+H), t_{R}: 1.237, 86.02% (Method-D)

### 3-methyl-4-((1-methylpyrrolidin-3-yl)oxy)aniline

To a degassed solution of 1-methyl-3-(2-methyl-4-nitrophenoxy)pyrrolidine (300 mg, 1.270 mmol) in MeOH (20 ml) was added 10% palladium on carbon (100 mg) under nitrogen atmosphere. The reaction mixture was stirred under hydrogen bladder pressure at room temperature for 16 h. The progress of the reaction was monitored by TLC and UPLC., the reaction mixture was filtered through a pad of celite to remove the catalyst and washed with MeOH (2×50 mL). The filtrate was concentrated under reduced pressure to get 3-methyl-4-((1-methylpyrrolidin-3-yl)oxy)aniline (200 mg, 0.970 mmol, 76% yield) as a black gummy solid, which was taken into the next step without further purification. LCMS (ESI) m/z=207.3 (M+H), t_{R}: 1.323, 74.62% (Method-B)

### N-(2-bromo-6-chlorophenyl)-2,4-dichloropyrimidine-5-carboxamide

To a stirred solution of 2,4-dichloropyrimidine-5-carbonyl chloride (500 mg, 2.36 mmol) in EtOAc (20 mL) was added 2-bromo-6-chloroaniline (488 mg, 2.36 mmol) followed by Amberlyst A21 (100 mg) at room temperature under nitrogen atmosphere. The reaction mixture was allowed to stir for 16 h. (TLC and UPLC), the mixture was filtered through sintered funnel and the filtrate was diluted with saturated aq. NaHCO₃ solution, and extracted with EtOAc (100 mL×2). The combined organic layer was dried over Na₂SO₄, filtered, and concentrated under reduced pressure. The resulting crude material was purified by flash chromatography (SiO₂/230-400 mesh; ~15% EtOAc/hexanes) to afford N-(2-bromo-6-chlorophenyl)-2,4-dichloropyrimidine-5-carboxamide (120 mg, 13% yield) as a brown solid. 1H-NMR (400 MHz, DMSO-d6): δ 10.86 (s, 1H), 9.01 (s, 1H), 7.79-7.76 (m, 1H), 7.68-7.66 (s, 1H), 7.37 (t, J=8.1 Hz, 1H). LCMS (ESI) m/z=377.8/379.8 (M−H), bromine isotopes, t_{R}: 1.864 min, 93.5% (Method-A).

### N-(2-bromo-6-chlorophenyl)-2-chloro-4-methoxypyrimidine-5-carboxamide

To a stirred solution of N-(2-bromo-6-chlorophenyl)-2,4-dichloropyrimidine-5-carboxamide (120 mg, 0.32 mmol) in THE (10 mL) at 0° C. was added sodium methanolate (17 mg, 0.32 mmol), and allowed to stir at rt for 16 h. (TLC and UPLC), the mixture was quenched with water and extracted with EtOAc (40 mL×2). The combined organic layer was dried over Na₂SO₄, filtered and concentrated under reduced pressure. The resulting crude material was purified by flash chromatography (SiO₂/100-200 mesh; ~10% EtOAc/hexanes) to afford N-(2-bromo-6-chlorophenyl)-2-chloro-4-methoxypyrimidine-5-carboxamide (102 mg, 86% yield) as a brown solid. 1H-NMR (400 MHz, DMSO-d6): δ 10.21 (s, 1H), 8.81 (s, 1H), 7.76-7.74 (m, 1H), 7.65-7.63 (m, 1H), 7.34 (t, J=8.4 Hz, 1H), 4.10 (s, 3H).

### N-(2-bromo-6-chlorophenyl)-4-methoxy-2-((3-methyl-4-((1-methylpyrrolidin-3-yl)oxy)phenyl)amino)pyrimidine-5-carboxamide

To a stirred solution of 3-methyl-4-((1-methylpyrrolidin-3-yl)oxy)aniline (150 mg, 0.727 mmol) in acetic acid (2 ml) was added N-(2-bromo-6-chlorophenyl)-2-chloro-4-methoxypyrimidine-5-carboxamide (274 mg, 0.727 mmol) at room temperature. The resulting reaction mixture was stirred at 25° C. for 16 h. The progress of the reaction was monitored by TLC and LCMS., the reaction mixture was diluted with water, extracted with 10% Methanol-DCM (2×50 mL), and washed with 10% aq. NaHCO₃ (50 mL). The combined organic layers were dried over Na₂SO₄, filtered, and concentrated under reduced pressure. The resulting crude residue was purified by prep. HPLC (X Select C18, Mobile phase A: 0.1% Formic Acid in H₂O, Mobile phase B: Acetonitrile) to get N-(2-bromo-6-chlorophenyl)-4-methoxy-2-((3-methyl-4-((1-methylpyrrolidin-3-yl)oxy)phenyl)amino)pyrimidine-5-carboxamide (55 mg, 0.101 mmol, 13.83% yield) as an off-white solid. LCMS (ESI) m/z=545.9 (M+H), t_{R}: 1.637, 98.16% (Method-D). ¹H-NMR (400 MHz, DMSO-d₆): δ 9.93 (s, 1H), 9.54 (s, 1H), 8.73 (s, 1H), 7.73 (d, J=1.20 Hz, 1H), 7.71 (d, J=0.80 Hz, 1H), 7.62-7.52 (m, 2H), 7.31 (t, J=8.00 Hz, 1H), 6.83 (d, J=9.20 Hz, 1H), 4.83-4.80 (m, 1H), 4.09 (s, 3H), 2.83-2.79 (m, 1H), 2.68-2.64 (m, 2H), 2.41-2.38 (m, 1H), 2.36 (s, 4H), 2.29 (s, 3H), 2.14-1.80 (m, 1H).

### Example 61. Synthesis of N-(2-bromo-6-chlorophenyl)-2-{4-[(R)-1-methyl-3-pyrrolidinyl]-3-toluidino}-4-methoxy-5-pyrimidinecarboxamide (Compound 192) and N-(2-bromo-6-chlorophenyl)-2-{4-[(S)-1-methyl-3-pyrrolidinyl]-3-toluidino}-4-methoxy-5-pyrimidinecarboxamide (Compound 193)

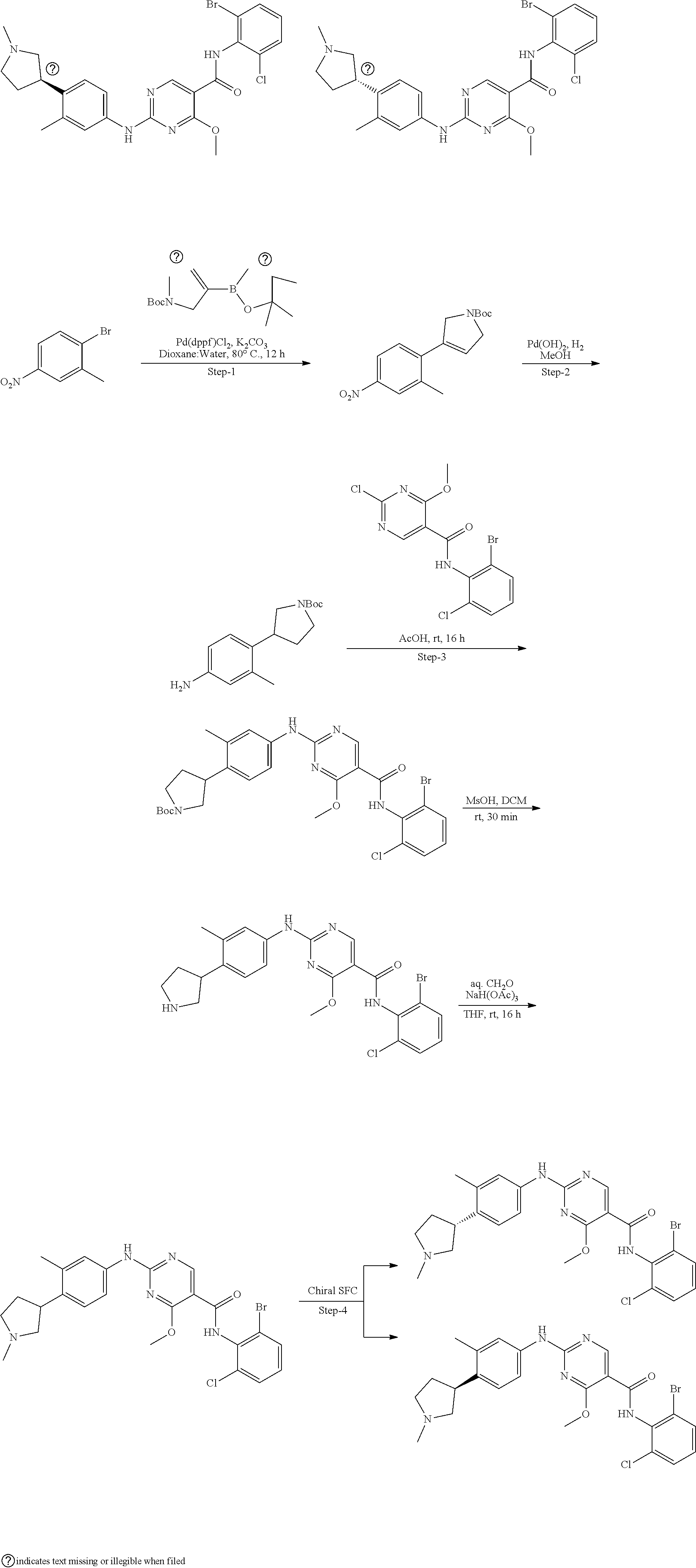

### tert-butyl 3-(2-methyl-4-nitrophenyl)-2,5-dihydro-1H-pyrrole-1-carboxylate

A degassed solution of 1-bromo-2-methyl-4-nitrobenzene (3 g, 13.89 mmol), K₃PO₄ (7.37 g, 34.7 mmol), tert-butyl 3-(4,4,5,5-tetramethyl-1,3,2-dioxaborolan-2-yl)-2,5-dihydro-1H-pyrrole-1-carboxylate (4.10 g, 13.89 mmol) and Pd(dppf)Cl₂ (1.016 g, 1.389 mmol) in 1,4-dioxane (45 ml) and water (5.000 ml) was allowed to stir at 80° C. for 16 h. The progress of the reaction was monitored by TLC after the completion of the reaction. The reaction mixture was diluted with water (100 mL) and extracted with 10% methanol-DCM (250 mL×3). The combined organic extract was washed with brine (150 mL), dried over anhydrous sodium sulphate, filtered, and concentrated under reduced pressure (bath temperature: 45° C.) to afford crude product. The crude product was purified by combi flash column chromatography (silica-gel, mesh size-100-200) using ethyl acetate-hexane (25-30%) as an eluent to obtain a tert-butyl 3-(2-methyl-4-nitrophenyl)-2,5-dihydro-1H-pyrrole-1-carboxylate (3.85 g, 12.52 mmol, 90% yield). LCMS (ESI) m/z=249.0 (M−56), t_{R}=2.14 min, 99.81% (Method A).

### tert-butyl 3-(4-amino-2-methylphenyl)pyrrolidine-1-carboxylate

To a degassed solution of tert-butyl 3-(2-methyl-4-nitrophenyl)-2,5-dihydro-1H-pyrrole-1-carboxylate (1.5 g, 4.93 mmol) in MeOH (40.0 ml), was added palladium hydroxide on carbon (0.277 g, 1.971 mmol) portion wise under hydrogen atmosphere. The reaction mixture was stirred for 12 h at room temperature. The progress of the reaction was monitored by TLC., the reaction mixture was filtered through a small pad of celite and washed with MeOH. The combined organic extract was concentrated to afford tert-butyl 3-(4-amino-2-methylphenyl)pyrrolidine-1-carboxylate 1.2 g, 4.08 mmol, 83% yield). LCMS (ESI) m/z=221 (M−56), t_{R}=2.10 min, 94% (Method D).

### tert-butyl 3-(4-((5-((2-bromo-6-chlorophenyl)carbamoyl)-4-methoxypyrimidin-2-yl)amino)-2-methylphenyl)pyrrolidine-1-carboxylate

To a stirred solution of tert-butyl 3-(4-amino-2-methylphenyl)pyrrolidine-1-carboxylate (528 mg, 1.910 mmol) in acetic acid (10 ml), N-(2-bromo-6-chlorophenyl)-2-chloro-4-methoxypyrimidine-5-carboxamide (600 mg, 1.591 mmol) was added at 25° C. The reaction mixture was stirred for 12 h. The progress of the reaction was monitored by TLC., acetic acid was evaporated under reduced pressure. The reaction mixture was diluted with sodium bicarbonate (50 mL) and extracted with 10% methanol-DCM (3×50 mL), dried over anhydrous sodium sulphate, filtered, and concentrated under reduced pressure (bath temperature: 45° C.) to afford crude product. The crude product was purified by combi flash column chromatography (silica-gel, mesh size-100-200) using ethyl acetate-hexane (30-40%) as an eluent to obtain a tert-butyl 3-(4-((5-((2-bromo-6-chlorophenyl)carbamoyl)-4-methoxypyrimidin-2-yl)amino)-2-methylphenyl)pyrrolidine-1-carboxylate (750 mg, 1.155 mmol, 72.6% yield). LCMS (ESI) m/z=618 (M+2), t_{R}=2.19 min, 94.87% (Method-A).

### N-(2-bromo-6-chlorophenyl)-4-methoxy-2-((3-methyl-4-(pyrrolidin-3-yl)phenyl)amino)pyrimidine-5-carboxamide

To a stirred solution of tert-butyl 3-(4-((5-((2-bromo-6-chlorophenyl)carbamoyl)-4-methoxypyrimidin-2-yl)amino)-2-methylphenyl)pyrrolidine-1-carboxylate (815 mg, 1.321 mmol) in methanesulphonic acid (762 mg, 7.93 mmol) at 0° C. The reaction mixture was stirred at room temperature for 2 h. The progress of the reaction was monitored by TLC after the completion of the reaction. The reaction mixture was diluted with sodium bicarbonate (100 mL) and extracted with 10% methanol-DCM (100 mL×3), dried over anhydrous sodium sulphate, filtered, and concentrated under reduced pressure to get N-(2-bromo-6-chlorophenyl)-4-methoxy-2-((3-methyl-4-(pyrrolidin-3-yl)phenyl)amino)pyrimidine-5-carboxamide (500 mg, 0.861 mmol, 65.2% yield). LCMS (ESI) m/z=518 (M−100), t_{R}=1.38 min, 89.49% (Method-A).

### N-(2-bromo-6-chlorophenyl)-4-methoxy-2-((3-methyl-4-(I-methylpyrrolidin-3-yl)phenyl)amino)pyrimidine-5-carboxamide

To a stirred solution of N-(2-bromo-6-chlorophenyl)-4-methoxy-2-((3-methyl-4-(pyrrolidin-3-yl)phenyl)amino)pyrimidine-5-carboxamide (100 mg, 0.193 mmol) in THE (20 ml) was added formaldehyde (79 mg, 0.967 mmol) at 25° C. The reaction mixture was stirred at 25° C. for 5 min and then sodium triacetoxyborohydride (120 mg, 0.580 mmol) was added portion-wise, under inert atmosphere. After the complete addition the reaction mixture was stirred at 25° C. for 10 min. The progress of reaction was monitored by TLC, after completion of reaction. The reaction mixture was quenched with TFA. Ammonia in methanol was added to neutralize the mixture. The reaction mixture was diluted with water (30 mL) and extracted with 10% methanol-DCM (20 mL×3). The combined organic extract was washed with sodium bicarbonate (150 mL), dried over anhydrous sodium sulphate, filtered, and concentrated under reduced pressure (bath temperature: 45° C.) to afford N-(2-bromo-6-chlorophenyl)-4-methoxy-2-((3-methyl-4-(1-methylpyrrolidin-3-yl)phenyl)amino)pyrimidine-5-carboxamide (70 mg, 0.129 mmol, 66.8% yield). LCMS (ESI) m/z=532 (M+2), t_{R}=1.53 min, 98.37% (Method-D).

### SFC Prep-Purification:

The racemic compound (120 mg) was separated via chiral SFC purification. Using the method I-CELLULOSE J -(250*30)mm, 5 μm CO₂: 0.5% IPAm in MeOH[80:20]. Pure fractions were collected and lyophilized to afford (S)—N-(2-bromo-6-chlorophenyl)-4-methoxy-2-((3-methyl-4-(1-methylpyrrolidin-3-yl)phenyl)amino)pyrimidine-5-carboxamide (50 mg) and (R)—N-(2-bromo-6-chlorophenyl)-4-methoxy-2-((3-methyl-4-(1-methylpyrrolidin-3-yl)phenyl)amino)pyrimidine-5-carboxamide (43.1 mg).

Analytical data for (S)—N-(2-bromo-6-chlorophenyl)-4-methoxy-2-((3-methyl-4-(1-methylpyrrolidin-3-yl)phenyl)amino)pyrimidine-5-carboxamide: LCMS (ESI) m/z=532 (M+2), t_{R}=1.36 min, 96.15% (Method A). ¹H-NMR (400 MHz, DMSO-d₆): δ 10.04 (s, 1H), 9.58 (s, 1H), 8.75 (s, 1H), 7.73 (dd, J=1.20, 8.20 Hz, 1H), 7.62-7.55 (m, 3H), 7.31 (dd, J=7.20, 8.20 Hz, 2H), 4.12 (s, 3H), 3.55-3.51 (m, 1H), 2.92 (t, J=8.00 Hz, 1H), 2.77-2.51 (m, 2H), 2.44 (s, 3H), 2.34-2.27 (m, 5H), 1.76 (q, J=6.40 Hz, 1H).

Analytical data for (R)—N-(2-bromo-6-chlorophenyl)-4-methoxy-2-((3-methyl-4-(1-methylpyrrolidin-3-yl)phenyl)amino)pyrimidine-5-carboxamide (43.1 mg): LCMS (ESI) m/z=532 (M+2), t_{R}. 1.37 min, 98.17% (Method A). ¹H-NMR (400 MHz, DMSO-d₆): δ 10.01 (s, 1H), 9.58 (s, 1H), 8.75 (s, 1H), 7.73 (dd, J=1.20, 8.20 Hz, 1H), 7.62-7.55 (m, 2H), 7.53 (s, 1H), 7.33-7.28 (m, 2H), 4.11 (s, 3H), 3.55-3.51 (m, 1H), 2.92 (t, J=8.00 Hz, 1H), 2.79 (t, J=8.40 Hz, 1H), 2.68-2.55 (m, 2H), 2.48-2.31 (m, 6H), 1.74-1.63 (m, 1H).

### Example 62. Synthesis of N-(2-bromo-6-chlorophenyl)-2-{4-[(R)-3-pyrrolidinyl]-3-toluidino}-4-methoxy-5-pyrimidinecarboxamide (Compound 194) and N-(2-bromo-6-chlorophenyl)-2-{4-[(S)-3-pyrrolidinyl]-3-toluidino}-4-methoxy-5-pyrimidinecarboxamide (Compound 195)

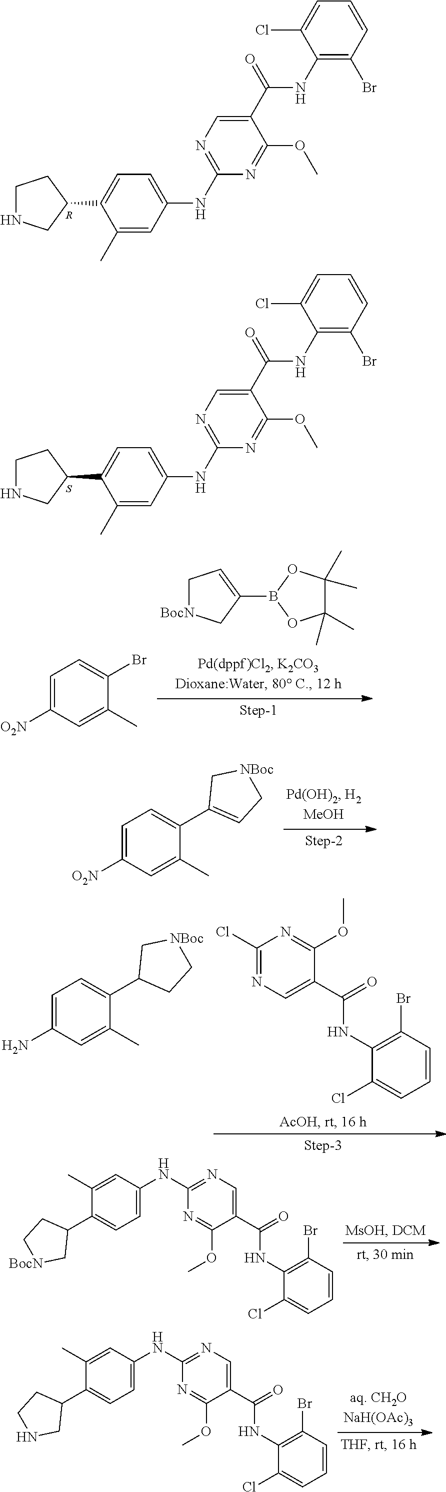

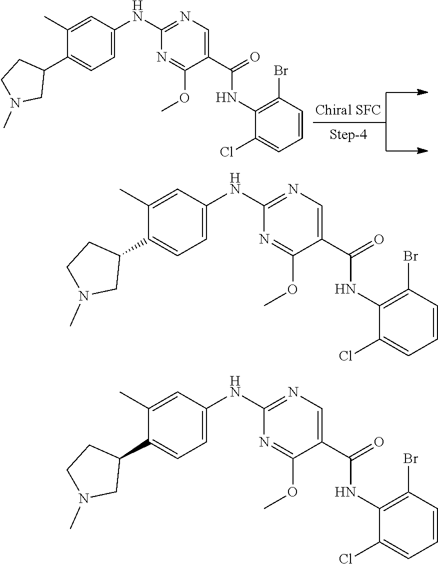

### tert-butyl 3-(2-methyl-4-nitrophenyl)-2,5-dihydro-1H-pyrrole-1-carboxylate

To a stirred solution of 1-bromo-2-methyl-4-nitrobenzene (4 g, 18.52 mmol) in 1,4-dioxane (24 ml) and water (16 ml) was added tert-butyl 3-(4,4,5,5-tetramethyl-1,3,2-dioxaborolan-2-yl)-2,5-dihydro-1H-pyrrole-1-carboxylate (5.47 g, 18.52 mmol) and K₂CO₃ (3.33 g, 55.5 mmol). The mixture was degassed by bubbling with nitrogen gas for 15 min. Pd(dppf)Cl₂ (0.677 g, 0.926 mmol) was added to the reaction mixture and stirred at 80° C. for 12 h. The progress of the reaction was monitored by TLC & UPLC., the reaction mixture was diluted with water (100 mL) and extracted with 10% methanol-DCM (3×150 mL). The combined organic extract was washed with brine (2×150 mL), dried over anhydrous sodium sulfate, filtered, and concentrated under reduced pressure (bath temperature: 45° C.) to afford crude product. Crude material was purified by combi flash column chromatography (silica-gel, mesh size-60-120) using ethyl acetate-hexane (25-30%) as an eluent to obtain tert-butyl 3-(2-methyl-4-nitrophenyl)-2,5-dihydro-1H-pyrrole-1-carboxylate (4 g, 12.97 mmol, 70.1% yield) as a brown viscous liquid.

### tert-butyl 3-(4-amino-2-methylphenyl)pyrrolidine-1-carboxylate

To a degassed solution of tert-butyl 3-(2-methyl-4-nitrophenyl)-2,5-dihydro-1H-pyrrole-1-carboxylate (800 mg, 2.63 mmol) in MeOH (30 ml) was added Pd(OH)₂ (36.4 mg, 0.26 mmol) portion-wise under inert atmosphere at 25° C. The reaction mixture was stirred at 25° C. for 16 h under a hydrogen atmosphere. The progress of the reaction was monitored by TLC & UPLC., the reaction mixture was filtered through celite bed, and the bed was washed with methanol (500 mL). The filtrate was concentrated under reduced pressure (bath temperature: 45° C.) to afford crude tert-butyl 3-(4-amino-2-methylphenyl)pyrrolidine-1-carboxylate (700 mg, 2.53 mmol, 96.35% yield). The crude compound was taken for the next step as it is. LCMS (ESI) m/z=221.1 [M−56], t_{R}=1.39 min, 98.99% (Method A).

### ethyl tert-butyl 3-(4-((5-((2-bromo-6-chlorophenyl)carbamoyl)-4-methoxypyrimidin-2-yl)amino)-2-methylphenyl)pyrrolidine-1-carboxylate

To a stirred solution of tert-butyl 3-(4-amino-2-methylphenyl)pyrrolidine-1-carboxylate (300 mg, 1.085 mmol) in Acetic Acid (4 ml) was added N-(2-bromo-6-chlorophenyl)-2-chloro-4-methoxypyrimidine-5-carboxamide (614 mg, 1.628 mmol) at 25° C. The reaction mixture was stirred at 25° C. under microwave irradiation. The progress of the reaction was monitored by TLC & UPLC., the reaction mixture was concentrated in vacuum to remove acetic acid. The reaction mixture was diluted with ice-cold water (50 mL) and extracted with solvent 10% Methanol: DCM (3×50 mL). The combined organic extract was washed with saturated sodium bicarbonate solution (100 mL). The organic layer was washed with brine (50 mL), dried over anhydrous sodium sulfate, filtered, and concentrated under reduced pressure (bath temperature: 45° C.) to afford crude compound. The crude product was purified by reverse phase column chromatography (C18 column) using acetonitrile-water (50-60%) as an eluent to obtain tert-butyl 3-(4-((5-((2-bromo-6-chlorophenyl)carbamoyl)-4-methoxypyrimidin-2-yl)amino)-2-methylphenyl)pyrrolidine-1-carboxylate (290 mg, 0.470 mmol, 43.3% yield) as a off white solid.

### Chiral SFC Purification

The racemic compound (290 mg) was separated via chiral SFC purification. Using the method I-CELLULOSE J -(250*30)mm, 5 μm CO₂: 0.5% IPAm in MeOH[80:20]. Pure fractions were collected and lyophilized to afford (R)—N-(2-bromo-6-chlorophenyl)-4-methoxy-2-((3-methyl-4-(pyrrolidin-3-yl)phenyl)amino)pyrimidine-5-carboxamide (143 mg) and (S)—N-(2-bromo-6-chlorophenyl)-4-methoxy-2-((3-methyl-4-(pyrrolidin-3-yl)phenyl)amino)pyrimidine-5-carboxamide (100 mg).

### (R)—N-(2-bromo-6-chlorophenyl)-4-methoxy-2-((3-methyl-4-(pyrrolidin-3-yl)phenyl)amino)pyrimidine-5-carboxamide

To a stirred solution of tert-butyl (R)-3-(4-((5-((2-bromo-6-chlorophenyl)carbamoyl)-4-methoxypyrimidin-2-yl)amino)-2-methylphenyl)pyrrolidine-1-carboxylate (143 mg, 0.232 mmol) in DCM (8 ml) at 0° C., was added methanesulfonic acid (0.090 ml, 1.391 mmol). The reaction was stirred at 0° C. for 2 h. The progress of the reaction was monitored by TLC & UPLC., the reaction mixture was diluted with ice-cold water (50 mL) and extracted with 10% Methanol: DCM solvent mixture (3×50 mL). The combined organic extract was washed with saturated sodium bicarbonate solution (50 mL). The organic layer was dried over anhydrous sodium sulfate, filtered, and concentrated under reduced pressure (bath temperature: 45° C.) to afford crude compound, which was lyophilized to get a (R)—N-(2-bromo-6-chlorophenyl)-4-methoxy-2-((3-methyl-4-(pyrrolidin-3-yl)phenyl)amino)pyrimidine-5-carboxamide (32.86 mg, 0.063 mmol, 27.2% yield) as a off-white solid. LCMS (ESI) m/z=516.1 (M+H), t_{R}=1.50 min, 98.97% (Method-D); ¹H-NMR (400 MHz, DMSO-d₆): δ 10.03 (s, 1H), 9.58 (s, 1H), 8.75 (s, 1H), 7.73 (dd, J=1.20, 8.00 Hz, 1H), 7.58-7.63 (m, 3H), 7.33-7.19 (m, 2H), 4.12 (s, 3H), 3.02-3.06 (m, 3H), 2.70-2.65 (m, 1H), 2.32-2.27 (m, 4H), 2.16-2.11 (m, 1H), 1.73-1.68 (m, 1H),

### (S)—N-(2-bromo-6-chlorophenyl)-4-methoxy-2-((3-methyl-4-(pyrrolidin-3-yl)phenyl)amino)pyrimidine-5-carboxamide

To a stirred solution of tert-butyl (S)-3-(4-((5-((2-bromo-6-chlorophenyl)carbamoyl)-4-methoxypyrimidin-2-yl)amino)-2-methylphenyl)pyrrolidine-1-carboxylate (100 mg, 0.232 mmol) in DCM (8 ml) at 0° C., was added methanesulfonic acid (0.090 ml, 1.391 mmol). The reaction was allowed to be stirred at 0° C. for 2 h. The progress of the reaction was monitored by TLC & UPLC., the reaction mixture was diluted with ice-cold water (50 mL) and extracted with 10% Methanol: DCM solvent mixture (3×50 mL). The combined organic extract was washed with saturated sodium bicarbonate solution (50 mL). The organic layer was dried over anhydrous sodium sulfate, filtered, and concentrated under reduced pressure (bath temperature: 45° C.) to afford crude compound, which was lyophilized to get (S)—N-(2-bromo-6-chlorophenyl)-4-methoxy-2-((3-methyl-4-(pyrrolidin-3-yl)phenyl)amino)pyrimidine-5-carboxamide (18.3 mg, 0.034 mmol, 22.26% yield) as an off-white solid. LCMS (ESI) m/z=516.2 (M+H), t_{R}=1.33 min, 97.68% (Method-A); ¹H-NMR (400 MHz, DMSO-d₆): δ 10.03 (s, 1H), 9.58 (s, 1H), 8.75 (s, 1H), 7.71 (dd, J=1.20, 8.00 Hz, 1H), 7.62-7.57 (m, 3H), 7.33-7.22 (m, 2H), 4.12 (s, 3H), 3.36-3.34 (m, 1H), 3.20-2.97 (m, 3H), 8.75 (s, 1H), 2.33-2.31 (m, 3H), 2.15-2.07 (m, 1H), 1.87 (s, 3H), 1.77-1.64 (m, 1H).

### Example 63. Synthesis of N-(2-bromo-6-chlorophenyl)-2-{1-[3-(dimethylamino)propyl]-4-pyrazolylamino}-4-ethoxy-5-pyrimidinecarboxamide (Compound 200)

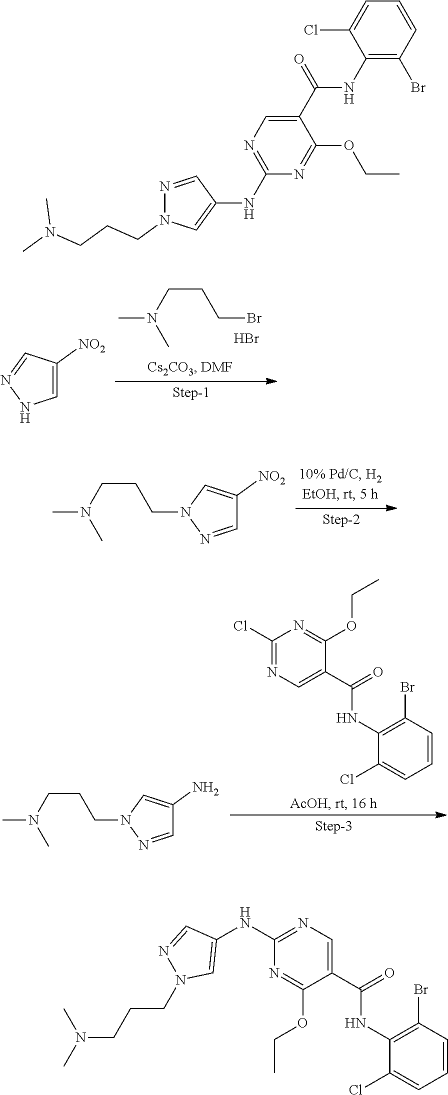

### N,N-dimethyl-3-(4-nitro-1H-pyrazol-1-yl)propan-1-amine

To a stirred solution of 4-nitro-1H-pyrazole (0.300 g, 2.65 mmol) in DMF (10 ml) was added Cs₂CO₃ (2.59 g, 7.96 mmol) followed by the addition of 3-bromo-N,N-dimethylpropan-1-amine hydrobromide (0.655 g, 2.65 mmol) and allowed to stir for 16 h. Progress of the reaction was monitored by TLC and LC-MS., reaction mass was diluted with water (50 mL) and extracted with 10% MeOH-DCM, and dried over anhydrous Na₂SO₄, filtered and distilled to get crude compound N,N-dimethyl-3-(4-nitro-1H-pyrazol-1-yl)propan-1-amine (270 mg, 0.749 mmol, 28.2% yield) as a yellow gummy compound. This compound, as such, is taken for the next step without any further purification.

### 1-(3-(dimethylamino)propyl)-1H-pyrazol-4-amine

To a stirred solution of N,N-dimethyl-3-(4-nitro-1H-pyrazol-1-yl)propan-1-amine (280 mg, 1.413 mmol) in EtOH (5 ml) was added Pd/C (235 mg, 1.413 mmol) under inert atmosphere, then stirred under H₂ bladder pressure for 5 h. Progress of the reaction was monitored by TLC and LCMS., the reaction mass was filtered through celite bed and then distilled to get crude compound 1-(3-(dimethyllamino)propyl)-1H-pyrazol-4-amine (230 mg, 1.362 mmol, 96% yield) as a brown gummy compound. As such, this compound was taken for the next step without any further purification. LCMS (ESI) m/z=169.1 (M+H), t_{R}:0.608 min, 92.16% (Method-D).

### N-(2-bromo-6-chlorophenyl)-2-chloro-4-ethoxypyrimidine-5-carboxamide

To a stirred solution of N-(2-bromo-6-chlorophenyl)-2,4-dichloropyrimidine-5-carboxamide (0.500 g, 1.311 mmol) in Ethanol (3 ml) was added Cs₂CO₃ (0.384 g, 1.180 mmol) at 0° C. Reaction was allowed to stir at 25° C. for 3 h. The progress of reaction was monitored by LCMS. water was added to the reaction mass, product was extracted with ethyl acetate the collected organic fraction was concentrated under vacuum to afford N-(2-bromo-6-chlorophenyl)-2-chloro-4-ethoxypyrimidine-5-carboxamide (0.500 g, 1.311 mmol 98% yield) as an off white solid. LCMS (ESI) m/z=392.1 (M+H), t_{R}. 1.99 min, 89.39% (Method C).

### N-(2-bromo-6-chlorophenyl)-2-((1-(3-(dimethylamino)propyl)-1H-pyrazol-4-yl)amino)-4-ethoxypyrimidine-5-carboxamide

To a stirred solution of 1-(3-(dimethylamino)propyl)-1H-pyrazol-4-amine (0.300 g, 1.783 mmol) in AcOH (2 mL) was added N-(2-bromo-6-chlorophenyl)-2-chloro-4-ethoxypyrimidine-5-carboxamide (0.628 g, 1.605 mmol) at room temperature and allowed to stir at 70° C. for 1 h. Progress of the reaction was monitored by TLC and LCMS., the solvent was evaporated under reduced pressure and diluted with water and extracted with 20% MeOH-DCM, dried over anhydrous Na₂SO₄, evaporated solvent under reduced pressure to get crude compound which was purified by prep HPLC (Column: X-select CSH C18, Mobile Phase A: 0.1% Formic acid in water, Mobile Phase B: Acetonitrile, Flowrate:15.0 mL/Min, Room temperature-10.8) to get N-(2-bromo-6-chlorophenyl)-2-((1-(3-(dimethylamino)propyl)-1H-pyrazol-4-yl)amino)-4-ethoxypyrimidine-5-carboxamide (128 mg, 0.242 mmol, 13.59% yield) as a yellow solid. LCMS (ESI) m/z=522.28 (M+H), t_{R}: 1.32 min, 99.21% (Method-A). ¹H-NMR (400 MHz, DMSO-d₆): δ 10.12 (s, 1H), 9.46-9.42 (m, 1H), 8.73-8.69 (m, 1H), 7.97-7.87 (m, 1H), 7.74 (d, J=1.20 Hz, 1H), 7.72 (d, J=1.20 Hz, 2H), 7.31 (t, J=8.00 Hz, 1H), 4.66-4.58 (m, 2H), 4.11-4.10 (m, 2H), 2.19-2.13 (m, 8H), 1.91-1.87 (m, 2H), 1.50-1.46 (m, 3H)

### Example 64. Synthesis of N-(2-bromo-6-chlorophenyl)-2-{1-[(S)-1-methyl-2-(methylamino)ethyl]-4-pyrazolylamino}-4-ethoxy-5-pyrimidinecarboxamide (Compound 201) and N-(2-bromo-6-chlorophenyl)-2-{1-[(R)-1-methyl-2-(methylamino)ethyl]-4-pyrazolylamino}-4-ethoxy-5-pyrimidinecarboxamide (Compound 203)

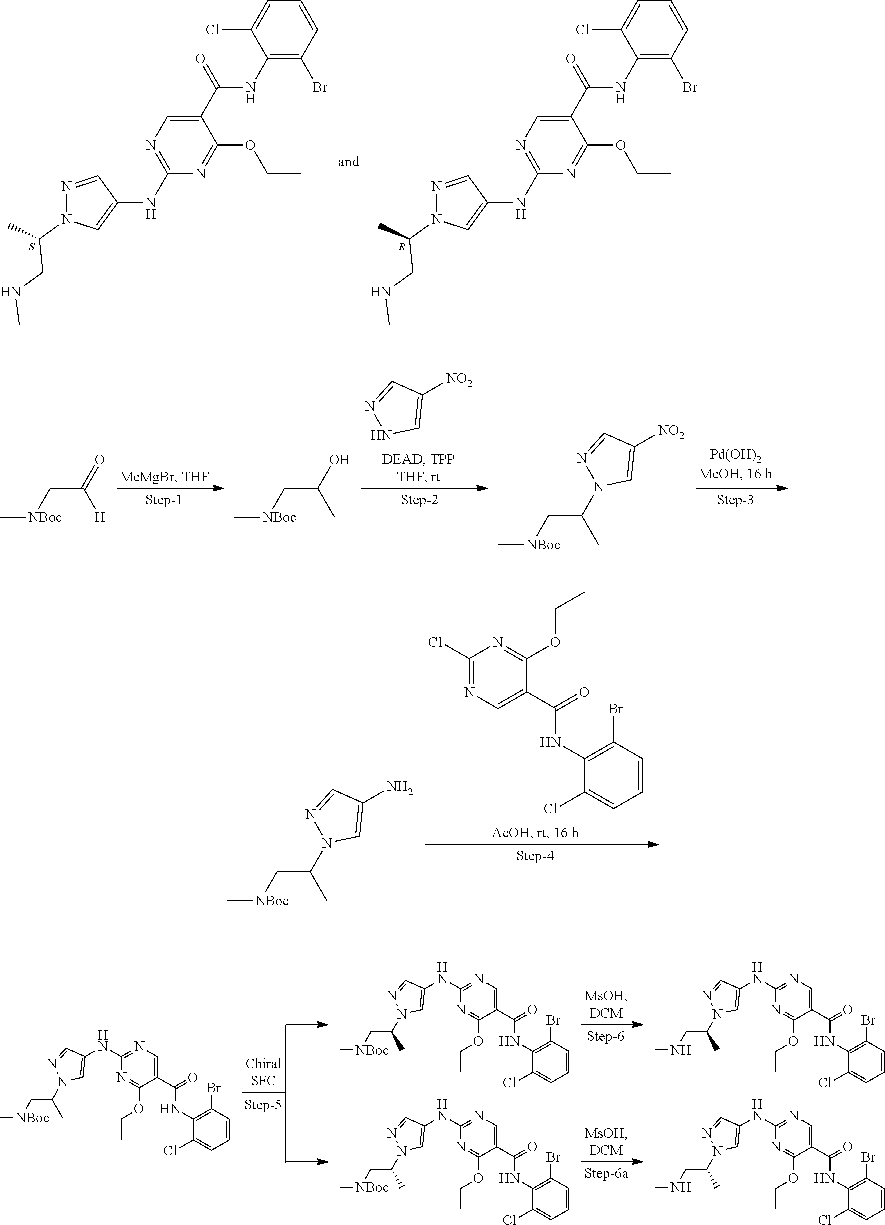

### tert-butyl (2-hydroxypropyl)(methyl)carbamate

To a stirred solution of tert-butyl methyl(2-oxoethyl)carbamate (2 g, 11.55 mmol) in THF (5 ml) was added MeMgBr (3.85 ml, 11.55 mmol) at 0° C. under inert atmosphere, then the resulting mixture was stirred at room temperature for 4 h. Progress of the reaction was monitored by TLC and LCMS., the reaction mixture was quenched with aq. NH₄Cl at 0° C. and extracted with ethyl acetate (2×100 mL). Combined organic layers and dried over anhydrous Na₂SO₄. The solvent was evaporated under reduced pressure to get tert-butyl (2-hydroxypropyl)(methyl)carbamate (2 g, 8.77 mmol, 76% yield) an oily liquid. LCMS (ESI) m/z=134.0 (M−56), t_{R}: 1.397 min, 83.85% (Method-A).

### tert-butyl methyl(2-(4-nitro-1H-pyrazol-1-yl)propyl)carbamate

To a stirred solution of 4-nitro-1H-pyrazole (1.2 g, 10.61 mmol), tert-butyl (2-hydroxypropyl)(methyl)carbamate (2.0 g, 10.61 mmol) in THE (5 ml) was added Triphenylphosphine (4.17 g, 15.92 mmol), DIAD (3.13 ml, 15.92 mmol) at 0° C. under inert atmosphere, then the resulting reaction mixture was stirred at room temperature for 16 h. The progress of the reaction was monitored by TLC and LCMS., the reaction mixture was concentrated under reduced pressure. The resulting crude residue was washed with water and extracted with ethyl acetate to get crude product that was purified by flash column chromatography (SiO₂/100-200 mesh; ethyl acetate-n-hexane, 30-40% as eluent) to get pure compound tert-butyl methyl(2-(4-nitro-1H-pyrazol-1-yl)propyl)carbamate (2 g, 5.49 mmol, 51.7% yield) as colourless gummy liquid. LCMS (ESI) m/z=285.2 (M+H), t_{R}: 1.71 min, 78.38% (Method-D).

### tert-butyl (2-(4-amino-1H-pyrazol-1-yl)propyl)(methyl)carbamate

To a stirred solution of tert-butyl methyl(2-(4-nitro-1H-pyrazol-1-yl)propyl)carbamate (2.0 g, 7.03 mmol) in MeOH (30 ml) was added 10% Pd/C (300 mg) under nitrogen atmosphere. The reaction mixture was stirred under a hydrogen atmosphere using hydrogen bladder pressure at room temperature for 16 h. The progress of the reaction was monitored by TLC and UPLC., the reaction mixture was filtered under a nitrogen atmosphere through a pad of celite to remove the catalyst, and the celite pad was washed with MeOH (150 mL). The filtrate was concentrated under reduced pressure to get tert-butyl (2-(4-amino-1H-pyrazol-1-yl)propyl)(methyl)carbamate (1.5 g, 4.60 mmol, 65.4% yield) as a colorless liquid, which was taken into the next step without further purification. LCMS (ESI) m/z=255.3 (M+H), t_{R}: 0.92 min, 78.01% (Method-A).

### tert-butyl (2-(4-((5-((2-bromo-6-chlorophenyl)carbamoyl)-4-ethoxypyrimidin-2-yl)amino)-1H-pyrazol-1-yl)propyl)(methyl)carbamate

To a stirred solution of tert-butyl (2-(4-amino-1H-pyrazol-1-yl)propyl)(methyl)carbamate (488 mg, 1.918 mmol) in acetic acid (5 ml) was added N-(2-bromo-6-chlorophenyl)-2-chloro-4-ethoxypyrimidine-5-carboxamide (500 mg, 1.279 mmol) at room temperature. The resulting reaction mixture was stirred at 25° C. for 16 h. The progress of the reaction was monitored by TLC and LCMS., the solvent was evaporated under reduced pressure. Then the reaction mixture was diluted with water and extracted with 10% MeOH-DCM (2×50 mL). The combined organic layers were washed with saturated sodium bicarbonate solution (30 ml), dried over Na₂SO₄, filtered, and concentrated under reduced pressure. The resulting crude residue was purified by chiral SFC (I-cellulose Z_0.5% IPAm in MeOH_30_0_4.lcd, Flow: 4.0000 mL/min, Co-Solvent: 30.0%, Oven Temperature: 40, Column Posiθon: 4: Column-4, BPR Pressure: 102.0 kgf/cm2) to obtain tert-butyl (R)-(2-(4-((5-((2-bromo-6-chlorophenyl)carbamoyl)-4-ethoxypyrimidin-2-yl)amino)-1H-pyrazol-1-yl)propyl)(methyl)carbamate (100 mg) as an off-white solid and tert-butyl (S)-(2-(4-((5-((2-bromo-6-chlorophenyl)carbamoyl)-4-ethoxypyrimidin-2-yl)amino)-1H-pyrazol-1-yl)propyl)(methyl)carbamate (100 mg) as an off-white solid.

### (S)—N-(2-bromo-6-chlorophenyl)-4-ethoxy-2-((1-(1-(methylamino)propan-2-yl)-1H-pyrazol-4-yl)amino)pyrimidine-5-carboxamide

To a stirred solution of tert-butyl (R)-(2-(4-((5-((2-bromo-6-chlorophenyl)carbamoyl)-4-ethoxypyrimidin-2-yl)amino)-1H-pyrazol-1-yl)propyl)(methyl)carbamate (100 mg, 0.164 mmol) in DCM (2 ml) was added methane sulfonic acid (10.66 μl, 0.164 mmol) at 0° C. under inert atmosphere. Then, the reaction was allowed to stir at 25° C. for 1 h. The progress of the reaction was monitored by TLC and LCMS after the completion of the reaction. The reaction mass was diluted with 10% MeOH in DCM (50 mL) and washed with saturated sodium bicarbonate solution (30 mL). The organic fraction was dried over anhydrous Na₂SO₄, and concentrated under reduced pressure. The resulting crude residue was purified by prep. HPLC (X Select C-8, 19×250), Mobile phase A: 10 mm ABC in Water, Mobile phase B: Acetonitrile) to get the (S)—N-(2-bromo-6-chlorophenyl)-4-ethoxy-2-((1-(1-(methylamino)propan-2-yl)-1H-pyrazol-4-yl)amino)pyrimidine-5-carboxamide (46 mg, 0.090 mmol, 54.5% yield) as an off-white solid. LCMS (ESI) m/z=508.0 (M+H), t_{R}: 1.349 min, 99.71% (Method-A). ¹H-NMR (400 MHz, DMSO-d₆): δ 10.12 (s, 1H), 9.29 (s, 1H), 8.73 (s, 1H), 7.91 (s, 1H), 7.72 (d, J=1.20 Hz, 1H), 7.70-7.61 (m, 2H), 7.29 (t, J=8.00 Hz, 1H), 4.66 (q, J=6.80 Hz, 2H), 4.41 (q, J=6.80 Hz, 1H), 2.91-2.86 (m, 1H), 2.79-2.75 (m, 1H), 2.29 (s, 3H), 1.48 (t, J=7.20 Hz, 3H), 1.42 (d, J=6.80 Hz, 3H).

### (R)—N-(2-bromo-6-chlorophenyl)-4-ethoxy-2-((1-(I-(methylamino)propan-2-yl)-1H-pyrazol-4-yl)amino)pyrimidine-5-carboxamide

To a stirred solution of tert-butyl (S)-(2-(4-((5-((2-bromo-6-chlorophenyl)carbamoyl)-4-ethoxypyrimidin-2-yl)amino)-1H-pyrazol-1-yl)propyl)(methyl)carbamate (100 mg, 0.164 mmol) in DCM (2 ml) was added methane sulfonic acid (10.66 μl, 0.164 mmol) at 0° C. under inert atmosphere. Then, the reaction was allowed to stir at 25° C. for 1 h. The progress of the reaction was monitored by TLC and LCMS. After the completion of the reaction, the reaction mass was diluted with 10% MeOH in DCM (50 mL) and washed with saturated sodium bicarbonate solution (30 mL). The collected organic fraction was dried over anhydrous Na₂SO₄, and concentrated under reduced pressure. The resulting crude residue was purified by prep. HPLC (X Select C-8, 19×250), Mobile phase A: 10 mm ABC in Water, Mobile phase B: Acetonitrile) to get the (R)—N-(2-bromo-6-chlorophenyl)-4-ethoxy-2-((1-(1-(methylamino)propan-2-yl)-1H-pyrazol-4-yl)amino)pyrimidine-5-carboxamide (46 mg, 0.090 mmol, 54.5% yield) as an off-white solid. LCMS (ESI) m/z=508.0 (M+H), t_{R}: 1.536 min, 99.42% (Method-A). ¹H-NMR (400 MHz, DMSO-d₆): δ 10.11 (s, 1H), 9.29 (s, 1H), 8.73 (s, 1H), 7.91 (s, 1H), 7.72 (d, J=1.20 Hz, 1H), 7.60-7.58 (m, 2H), 7.29 (t, J=8.00 Hz, 1H), 4.66 (q, J=6.80 Hz, 2H), 4.41 (q, J=6.80 Hz, 1H), 2.91-2.86 (m, 1H), 2.79-2.75 (m, 1H), 2.28 (s, 3H), 1.48 (t, J=7.20 Hz, 3H), 1.42 (d, J=6.80 Hz, 3H)

### Example 65. Synthesis of N-(2-bromo-6-chlorophenyl)-4-methoxy-2-[p-(4-methyl-1,4-diazepan-1-yl)phenylamino]-5-pyrimidinecarboxamide (Compound 206)

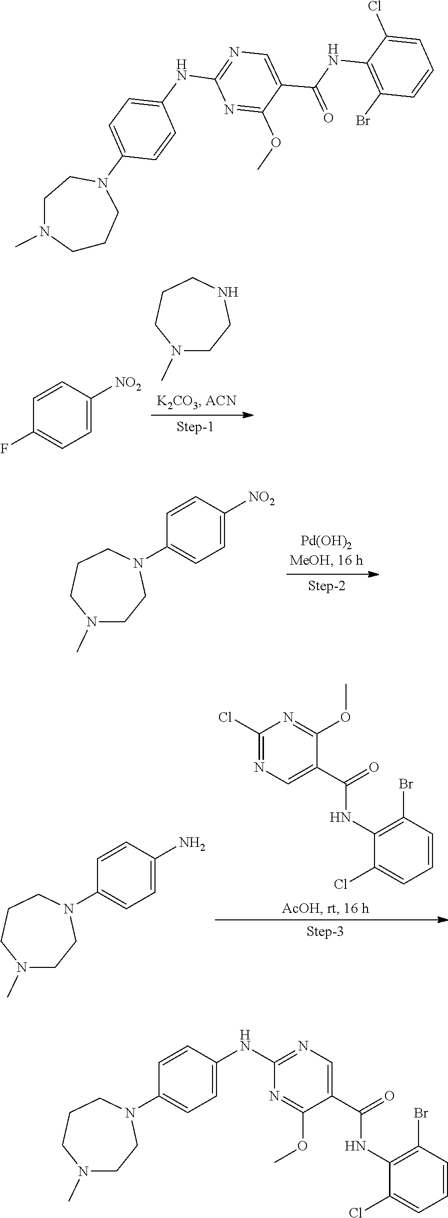

### 1-methyl-4-(4-nitrophenyl)-1,4-diazepane

To a stirred solution of 1-fluoro-4-nitrobenzene (1.0 g, 7.09 mmol) in acetonitrile (20 ml) was added K₂CO₃ (2.449 g, 17.72 mmol) and 1-methyl-1,4-diazepane (0.971 ml, 8.50 mmol) at room temperature under inert atmosphere and the resulting mixture was allowed to stir for 16 h at 80° C. Progress of the reaction was monitored by TLC and LCMS., the solvent was removed under reduced pressure and the residue diluted with water and extracted with ethyl acetate. The organic phase was dried over Na₂SO₄, filtered, and evaporated under reduced pressure to get a crude compound which was purified by flash column chromatography (SiO₂/100-200 mesh; MeOH-DCM, 0-10% as eluent) to obtain 1-methyl-4-(4-nitrophenyl)-1,4-diazepane (1.1 g, 2.85 mmol, 40.2% yield) as a yellow solid.

### 4-(4-methyl-1,4-diazepan-1-yl)aniline

To a stirred solution of 1-methyl-4-(4-nitrophenyl)-1,4-diazepane (2.0 g, 8.50 mmol) in EtOH (10 ml) was added Pd/C (1.0 g, 8.50 mmol). The mixture was allowed to stir under H₂ bladder pressure for 16 h at room temperature. Progress of the reaction was monitored by TLC and LCMS., the reaction mass was filtered through celite bed and washed with MeOH (2×200 mL), and the collected filtrate distilled to get crude compound 4-(4-methyl-1,4-diazepan-1-yl)aniline (1.5 g, 7.31 mmol, 86% yield) as a yellow gummy compound. This crude compound, as such, is taken for the next step without any further purification.

### N-(2-bromo-6-chlorophenyl)-4-methoxy-2-((4-(4-methyl-1,4-diazepan-1-yl)phenyl)amino)pyrimidine-5-carboxamide

To a stirred solution of 4-(4-methyl-1,4-diazepan-1-yl)aniline (0.300 g, 1.461 mmol) in AcOH (5 ml) was added N-(2-bromo-6-chlorophenyl)-2-chloro-4-methoxypyrimidine-5-carboxamide (0.496 g, 1.315 mmol) and allowed to stir for 1 h at 70° C. Progress of the reaction was monitored by TLC and LCMS., solvent was removed under reduced pressure and diluted with water and extracted with ethyl acetate (2×100 mL). The combined organic layers was dried over anhydrous Na₂SO₄, filtered, evaporated solvent under reduced pressure to get the crude compound which was purified by prep HPLC (Column: X-select CSH C18, Mobile Phase A: Ammonium acetate in water, Mobile Phase B: Acetonitrile, Flowrate:15.0 mL/Min, Room temperature-10.8) to get N-(2-bromo-6-chlorophenyl)-4-methoxy-2-((4-(4-methyl-1,4-diazepan-1-yl)phenyl)amino)pyrimidine-5-carboxamide (18.5 mg, 0.034 mmol, 2.2% yield) as a yellow solid compound. LCMS (ESI) m/z=545.3 (M+H), t_{R}:1.51 min, 95.20% (Method-D). ¹H-NMR (400 MHz, DMSO-d₆): δ 9.87-9.86 (m, 1H), 9.50 (s, 1H), 8.70 (s, 1H), 7.73 (d, J=1.20 Hz, 1H), 7.71-7.53 (m, 3H), 7.30 (t, J=8.00 Hz, 1H), 6.68 (d, J=9.20 Hz, 2H), 4.08 (s, 3H), 3.51-3.44 (m, 4H), 2.67-2.59 (m, 2H), 2.45-2.34 (m, 2H), 2.33 (s, 3H), 1.91-1.88 (m, 2H)

### Example 66. Synthesis of N-(2-bromo-6-chlorophenyl)-2-{4-[(S)-1-methyl-4-azepanyl]-3-toluidino}-4-methoxy-5-pyrimidinecarboxamide (Compound 208)

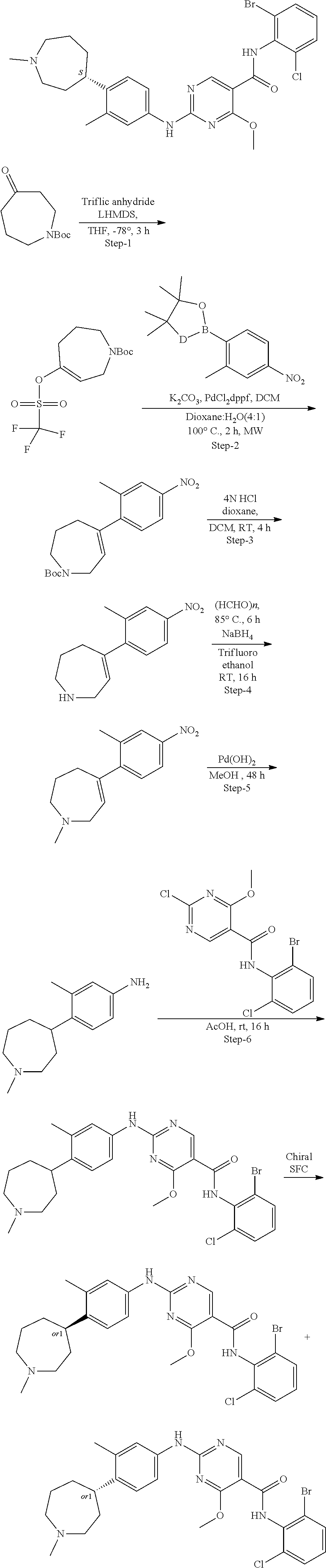

### tert-butyl 5-(((trifluoromethyl)sulfonyl)oxy)-2,3,4,7-tetrahydro-1H-azepine-1-carboxylate

To a stirred solution of tert-butyl 4-oxoazepane-1-carboxylate (6 g, 28.1 mmol) in THF (100 ml) at −78° C., LiHMDS (1M in THF) (56.3 ml, 56.3 mmol) was added in the reaction mixture dropwise over a period of 15 min and stirred at −78° C. for 1 h. Triflic anhydride (6.61 ml, 39.4 mmol) was added dropwise in the reaction mixture. The reaction mixture was allowed to be stirred at −78° C. for 3 h. The progress of the reaction was monitored by TLC. The reaction mixture was quenched in aqueous ammonium chloride solution (100 ml) and diluted with water (300 mL), extracted with solvent ethyl acetate (100 mL×3). and the combined organic extract was washed with brine (200 mL), dried over anhydrous sodium sulfate, filtered, and concentrated under reduced pressure (bath temperature: 45° C.) to afford crude compound. The crude product was purified by flash column chromatography (silica-gel, mesh size 60-120) using ethyl acetate/hexane (20 to 22%) as an eluent to obtain desired product tert-butyl 5-(((trifluoromethyl)sulfonyl)oxy)-2,3,4,7-tetrahydro-1H-azepine-1-carboxylate (1.3 g, 3.76 mmol, 13.38% yield) as a light gum. ¹H-NMR (400 MHz, DMSO-d₆): δ 6.06-6.04 (t, J=10.4 Hz, 1H), 3.90 (s, 2H), 3.49-3.46 (t, J=12 Hz, 2H), 2.55-2.50 (m, 2H), 1.88-1.85 (d, J=4.8 Hz, 2H), 1.40 (s, 9H).

### tert-butyl 5-(2-methyl-4-nitrophenyl)-2,3,4,7-tetrahydro-H-azepine-1-carboxylate

To a stirred solution of 4,4,5,5-tetramethyl-2-(2-methyl-4-nitrophenyl)-1,3,2-dioxaborolane (0.450 g, 1.710 mmol) and tert-butyl 5-(((trifluoromethyl)sulfonyl)oxy)-2,3,4,7-tetrahydro-1H-azepine-1-carboxylate (0.650 g, 1.881 mmol) in dioxane (12 mL) and water (3 mL), potassium carbonate (0.708 g, 5.13 mmol) was added in the reaction mixture at 25° C. The reaction mixture was degassed with nitrogen for 15 minutes. Pd(dppf)Cl₂ DCM Complex (0.140 g, 0.171 mmol) was added and the reaction mixture was stirred at 100° C. for 2 h with microwave irradiation. The progress of the reaction was monitored by TLC after the completion of the reaction. The reaction mixture was filtered through celite bed, the bed was washed with ethyl acetate (100 mL), and the filtrate was concentrated under reduced pressure. The residue was dissolved in ethyl acetate (200 mL) and washed with water (100 mL) and brine (100 mL). The organic extract was dried over anhydrous sodium sulphate, filtered, and concentrated under reduced pressure (bath temperature: 45° C.) to afford crude compound. The crude product was purified by flash column chromatography (silica-gel, mesh size 60-120) using ethyl acetate/hexane (15 to 20%) as an eluent to obtain desired product tert-butyl 5-(2-methyl-4-nitrophenyl)-2,3,4,7-tetrahydro-1H-azepine-1-carboxylate (600 mg, 1.534 mmol, 90% yield) as a brown solid. LCMS (ESI) m/z=233.29 (M−100), t_{R}: 2.24 min, 83.47% (Method-A).

### 5-(2-methyl-4-nitrophenyl)-2,3,4,7-tetrahydro-1H-azepine

To a stirred solution of tert-butyl 5-(2-methyl-4-nitrophenyl)-2,3,4,7-tetrahydro-1H-azepine-1-carboxylate (600 mg) in DCM (15 mL), 4 M HCl was added in the reaction mixture at 0° C. The reaction mixture was stirred at room temperature for 4 h. The progress of the reaction was monitored by TLC. The reaction mixture was concentrated under reduced pressure to afford 5-(2-methyl-4-nitrophenyl)-2,3,4,7-tetrahydro-1H-azepine (425 mg, 1.793 mmol) as a brown solid. LCMS (ESI) m/z=233.66 (M+1), t_{R}: 1.01 min, 98.11% (Method-A).

### 1-methyl-5-(2-methyl-4-nitrophenyl)-2,3,4,7-tetrahydro-1H-azepine

To a stirred solution of 5-(2-methyl-4-nitrophenyl)-2,3,4,7-tetrahydro-1H-azepine (425 mg, 1.830 mmol) in trifluoroethanol (10 mL), paraformaldehyde (220 mg, 7.32 mmol) was added. The reaction mixture was stirred at 85° C. for 6 h. The reaction mixture was cooled to 25° C. Sodium borohydride (277 mg, 7.32 mmol) was added to the reaction mixture and stirred at room temperature for 16 h. The progress of the reaction was monitored by TLC after the completion of the reaction. The reaction mixture was concentrated over the vacuum. The crude was dissolved in water (50 ml) and extracted with DCM (100 ml). The organic layers were collected, dried over sodium sulfate and then concentrated over vacuum to afford crude compound 1-methyl-5-(2-methyl-4-nitrophenyl)-2,3,4,7-tetrahydro-1H-azepine (400 mg, 1.413 mmol, 77% yield) as a brown gum. LCMS (ESI) m/z=247.29 (M+1), t_{R}: 0.99 min, 86.58% (Method-A).

### 3-methyl-4-(1-methylazepan-4-yl)aniline

To a stirred solution of 1-methyl-5-(2-methyl-4-nitrophenyl)-2,3,4,7-tetrahydro-1H-azepine (400 mg, 1.624 mmol) in methanol (20 mL), palladium hydroxide (400 mg, 0.285 mmol) was added. The reaction mixture was stirred at 25° C. for 48 h under a hydrogen atmosphere (balloon). The progress of the reaction was monitored by LCMS. The reaction mixture was filtered through celite bed; and the filtrate was collected and concentrated over vacuum to afford crude compound. The crude product was purified by flash column chromatography (silica-gel, mesh size 60-120) using methanol-DCM (10 to 15%) as an eluent to obtain desired product 3-methyl-4-(1-methylazepan-4-yl)aniline (135 mg, 0.439 mmol, 27.0% yield) as a brown gum.

### N-(2-bromo-6-chlorophenyl)-4-methoxy-2-((3-methyl-4-(I-methylazepan-4-yl)phenyl)amino)pyrimidine-5-carboxamide

To a stirred solution of 3-methyl-4-(1-methylazepan-4-yl)aniline (135 mg, 0.618 mmol) in acetic acid (5 mL), N-(2-bromo-6-chlorophenyl)-2-chloro-4-methoxypyrimidine-5-carboxamide (233 mg, 0.618 mmol) was added. The reaction mixture was stirred at room temperature for 16 h. The progress of the reaction was monitored by UPLC. The reaction mixture was concentrated under vacuum. The crude was dissolved in DCM and quenched in aq. sodium bicarbonate solution. The mixture was extracted with DCM. Organic layers were dried over sodium sulfate and concentrated under vacuum to afford crude compound. The crude compound was purified by prep-HPLC. Pure fractions were collected and lyophilized for to afford a pure compound (40 mg) white solid. LCMS (ESI) m/z=558.0 (M+1), t_{R}: 1.41 min, 95.09% (Method-A).

### Separation of (R)—N-(2-bromo-6-chlorophenyl)-4-methoxy-2-((3-methyl-4-(1-methylazepan-4-yl)phenyl)amino)pyrimidine-5-carboxamide and (S)—N-(2-bromo-6-chlorophenyl)-4-methoxy-2-((3-methyl-4-(1-methylazepan-4-yl)phenyl)amino)pyrimidine-5-carboxamide

After prep purification, 40 mg pure compound was purified by Chiral SFC, using the method I Cellulose-B_0.5% IPAm in MeOH_30_0_2.1.lcd. Pure fractions were collected and concentrated under vacuum to reduce the volume of solvent, then lyophilized to get dry and pure compounds (R)—N-(2-bromo-6-chlorophenyl)-4-methoxy-2-((3-methyl-4-(1-methylazepan-4-yl)phenyl)amino)pyrimidine-5-carboxamide (7.3 mg) and (S)—N-(2-bromo-6-chlorophenyl)-4-methoxy-2-((3-methyl-4-(1-methylazepan-4-yl)phenyl)amino)pyrimidine-5-carboxamide (6.2 mg).

Analytical Data (R)—N-(2-bromo-6-chlorophenyl)-4-methoxy-2-((3-methyl-4-(1-methylazepan-4-yl)phenyl)amino)pyrimidine-5-carboxamide: LCMS (ESI) m/z=560.13 (M+1), t_{R}: 1.42 min, 83.81% (Method-A). ¹H-NMR (400 MHz, DMSO-d6): δ 10.01 (s, 1H), 9.57 (s, 1H), 7.73 (dd, J=1.20, 8.00 Hz, 1H), 7.67-7.60 (m, 3H), 7.31 (t, J=8.00 Hz, 1H), 7.21 (dd, J=8.00, 24.00 Hz, 1H), 4.13 (s, 3H), 3.54 (m, 1H), 3.15-3.02 (m, 1H), 2.71-2.56 (m, 5H), 2.42 (s, 3H), 2.33 (s, 3H), 1.81-1.73 (m, 6H).

Analytical Data (S)—N-(2-bromo-6-chlorophenyl)-4-methoxy-2-((3-methyl-4-(1-methylazepan-4-yl)phenyl)amino)pyrimidine-5-carboxamide: LCMS (ESI) m/z=560.21 (M+1), t_{R}: 1.42 min, 85.89% (Method-A). ¹H-NMR (400 MHz, DMSO-d6): δ 10.00 (s, 1H), 9.57 (s, 1H), 8.76 (s, 1H), 7.64-7.54 (m, 3H), 7.33-7.29 (m, 1H), 7.19-7.15 (m, 1H), 2.72-2.70 (m, 1H), 2.67-2.56 (m, 4H), 2.37 (s, 3H), 2.31 (s, 3H), 1.79-1.67 (m, 6H).

### Example 67. Synthesis of N-(2-bromo-6-chlorophenyl)-4-methoxy-2-{p-(9-methyl-3,9-diaza-3-spiro[5.5]undecyl)phenylamino}-5-pyrimidinecarboxamide (Compound 209)

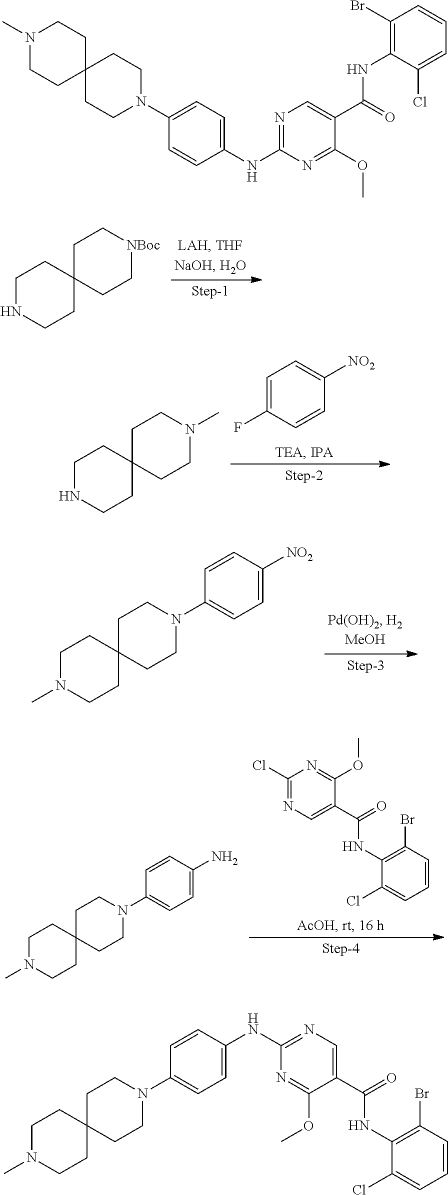

### 3-methyl-3,9-diazaspiro[5.5]undecane

To a stirred solution of tert-butyl 3,9-diazaspiro[5.5]undecane-3-carboxylate (500 mg, 1.966 mmol) in THF (5 ml) was added lithium aluminum hydride (1.966 ml, 3.93 mmol) at 0° C. The resulting reaction mixture was stirred at room temperature for 1 h. The progress of the reaction was monitored by TLC and LCMS., the reaction was quenched with 10% NaOH solution; a solid suspension was formed that was filtered through a Buchner funnel. The collected filtrate was concentrated under reduced pressure to get the crude compound 3-methyl-3,9-diazaspiro[5.5]undecane (300 mg, 1.785 mmol, 90% yield) as a gummy solid. This compound, as such, is taken for the next step without purification.

### 3-methyl-9-(4-nitrophenyl)-3,9-diazaspiro[5.5]undecane

To a stirred solution of 3-methyl-3,9-diazaspiro[5.5]undecane (300 mg, 1.783 mmol) in 2-propanol (3 ml) was added triethylamine (0.293 ml, 2.139 mmol) and 1-fluoro-4-nitrobenzene (252 mg, 1.783 mmol) at 25° C. under inert atmosphere. Then the reaction was allowed to stir at 150° C. in a microwave for 20 min. The progress of the reaction was monitored by TLC and LCMS., it was cooled to 0° C. 10 ml of cooled water was added causing the product to solidify. It was filtered through a Buchner funnel and washed with water, then dried under in vacuum to get the compound 3-methyl-9-(4-nitrophenyl)-3,9-diazaspiro[5.5]undecane (170 mg, 0.493 mmol, 27.7% yield) as an off-white solid. LCMS (ESI) m/z=290.4 (M+H), t_{R}: 1.11 min, 84.03% (Method-A)

### 4-(9-methyl-3,9-diazaspiro[5.5]undecan-3-yl)aniline

To a degassed solution of 3-methyl-9-(4-nitrophenyl)-3,9-diazaspiro[5.5]undecane (150 mg, 0.518 mmol) in MeOH (20 ml) was added 10% Pd/C (50 mg) under nitrogen atmosphere. Then, the reaction mixture was stirred under a hydrogen atmosphere using hydrogen bladder pressure at room temperature for 16 h. The progress of the reaction was monitored by TLC and UPLC., the reaction mixture was filtered under a nitrogen atmosphere through a pad of celite to remove the catalyst. The celite pad was washed with MeOH (50 mL). The filtrate was concentrated under reduced pressure to get 4-(9-methyl-3,9-diazaspiro[5.5]undecan-3-yl)aniline (130 mg, 0.456 mmol, 88% yield) as a black solid, which was taken into the next step without further purification.

### N-(2-bromo-6-chlorophenyl)-4-methoxy-2-((4-(9-methyl-3,9-diazaspiro[5.5]undecan-3-yl)phenyl)amino)pyrimidine-5-carboxamide

To a stirred solution of 4-(9-methyl-3,9-diazaspiro[5.5]undecan-3-yl)aniline (103 mg, 0.398 mmol) in acetic acid (2 mL) was added N-(2-bromo-6-chlorophenyl)-2-chloro-4-methoxypyrimidine-5-carboxamide (150 mg, 0.398 mmol) at room temperature. The resulting reaction mixture was stirred at 25° C. for 16 h. The progress of the reaction was monitored by TLC and LCMS., the solvent was evaporated under reduced pressure to get a crude residue, which was diluted with water and extracted with 10% MeOH-DCM (2×50 mL). The organic layer was washed with saturated sodium bicarbonate (30 mL) and dried over Na₂SO₄, filtered, and concentrated under reduced pressure. The resulting crude residue was purified by prep. HPLC (X Select C18, 19×250, Mobile phase A: 10 mM ammonium acetate in water) to get the pure compound N-(2-bromo-6-chlorophenyl)-4-methoxy-2-((4-(9-methyl-3,9-diazaspiro[5.5]undecan-3-yl)phenyl)amino)pyrimidine-5-carboxamide (13 mg, 0.021 mmol, 6.37% yield) as an pale yellow solid. LCMS (ESI) m/z=599.0 (M+H), t_{R}: 1.531 min, 95.72% (Method-D). ¹H-NMR (400 MHz, DMSO-d₆): δ 9.92 (s, 1H), 9.53 (s, 1H), 8.72 (s, 1H), 7.73 (d, J=0.80 Hz, 1H), 7.61 (t, J=6.80 Hz, 3H), 7.31 (t, J=8.00 Hz, 1H), 6.92 (d, J=8.80 Hz, 2H), 4.08 (s, 3H), 3.10-3.07 (m, 4H), 2.28 (s, 4H), 2.16 (s, 3H), 1.76 (s, 1H), 1.68-1.54 (m, 4H), 1.48-1.45 (m, 4H).

### Example 68. Synthesis of N-(2-bromo-6-chlorophenyl)-2-{4-[(R)-3-pyrrolidinyloxy-3-toluidino}-4-methoxy-5-pyrimidinecarboxamide (Compound 210)

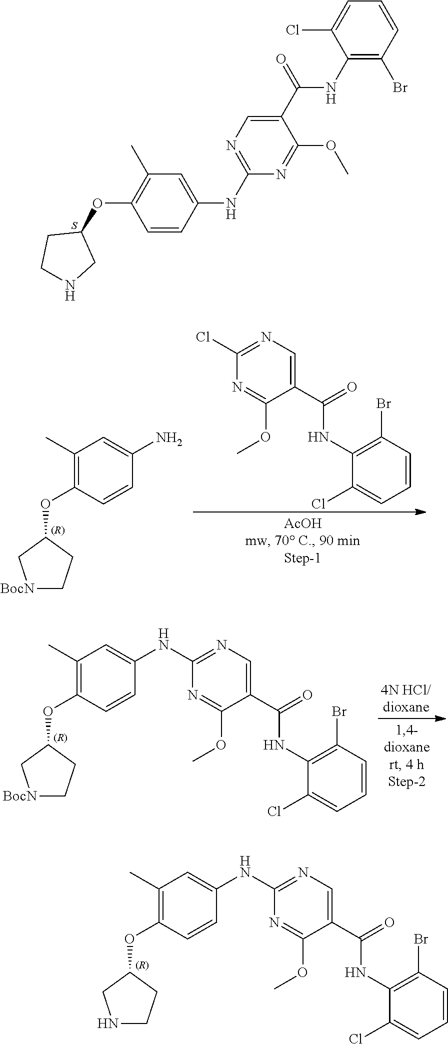

### tert-butyl (R)-3-(4-((5-((2-bromo-6-chlorophenyl)carbamoyl)-4-methoxypyrimidin-2-yl)amino)-2-methylphenoxy)pyrrolidine-1-carboxylate

To a stirred solution of N-(2-bromo-6-chlorophenyl)-2-chloro-4-methoxypyrimidine-5-carboxamide (250 mg, 0.663 mmol) in acetic acid (8 mL) was added tert-butyl (R)-3-(4-amino-2-methylphenoxy)pyrrolidine-1-carboxylate (213 mg, 0.729 mmol) at room temperature. The resulting reaction mixture was subjected to microwave irradiation at 70° C. for 1.5 h. The reaction was monitored by TLC., the reaction mixture was diluted with water and extracted with 10% Methanol-DCM (50 mL×2). The combined organic layer was dried over Na₂SO₄, filtered and concentrated under reduced pressure to get tert-butyl (R)-3-(4-((5-((2-bromo-6-chlorophenyl)carbamoyl)-4-methoxypyrimidin-2-yl)amino)-2-methylphenoxy)pyrrolidine-1-carboxylate (350 mg) as an off white solid which was forwarded to the next step without any further purification.

### (R)—N-(2-bromo-6-chlorophenyl)-4-methoxy-2-((3-methyl-4-(pyrrolidin-3-yloxy)phenyl)amino)pyrimidine-5-carboxamide

To a stirred solution of tert-butyl (R)-3-(4-((5-((2-bromo-6-chlorophenyl)carbamoyl)-4-methoxypyrimidin-2-yl)amino)-2-methylphenoxy)pyrrolidine-1-carboxylate (350 mg, 0.553 mmol) in 1,4-dioxane (5 mL), was added 4N HCl in 1,4-dioxane (2 mL) at 0° C. The resulting reaction mixture was stirred at room temperature for 8 h. The reaction was monitored by TLC., the reaction mixture was concentrated under reduced pressure. The resulting crude residue was purified by prep. HPLC (X Select C18, 19×150, mobile phase A: 0.1M FA in H₂O, mobile phase B: acetonitrile) to get (R)—N-(2-bromo-6-chlorophenyl)-4-methoxy-2-((3-methyl-4-(pyrrolidin-3-yloxy)phenyl)amino)pyrimidine-5-carboxamide (46 mg, 0.085 mmol, 15.46% yield) as an off-white solid. LCMS (ESI) m/z=532.2 (M+H), t_{R}: 1.40 min, 95.18% (Method-A). ¹H-NMR (400 MHz, DMSO-d₆, at 80° C.): δ 9.63 (s, 1H), 9.37 (s, 1H), 8.73 (s, 1H), 7.71 (d, J=1.20 Hz, 1H), 7.59-7.50 (m, 3H), 7.29 (t, J=8.00 Hz, 1H), 6.90 (d, J=8.80 Hz, 1H), 4.86-4.83 (m, 1H), 4.11 (s, 3H), 2.94-2.87 (m, 4H), 2.17 (s, 3H), 2.10-1.89 (m, 1H), 1.87-1.84 (m, 1H).

### Example 69. Synthesis of N-(2-bromo-6-chlorophenyl)-2-{4-[(R)-1-methyl-3-pyrrolidinyloxy]-3-toluidino}-4-methoxy-5-pyrimidinecarboxamide (Compound 211)

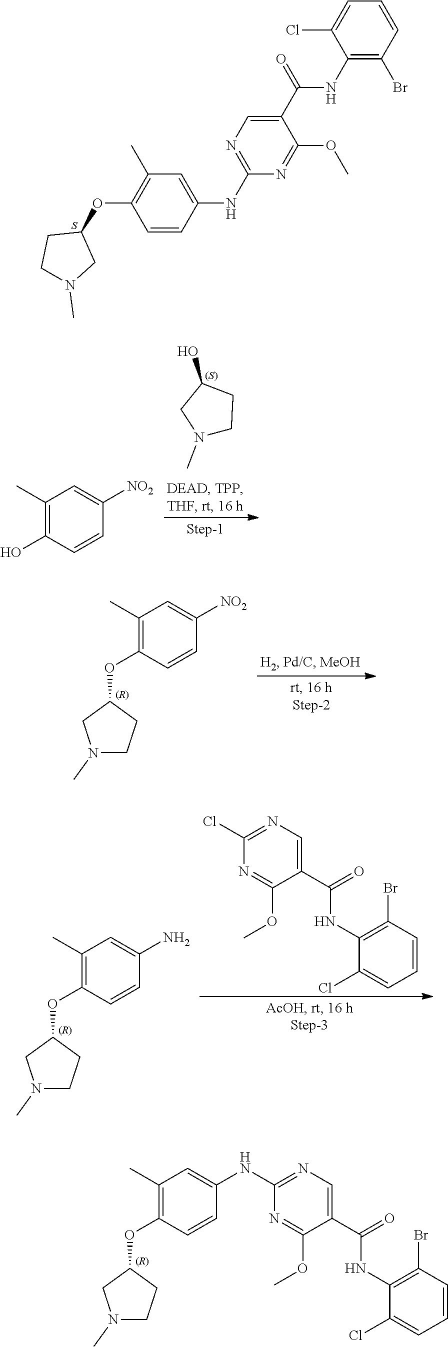

### (R)-1-methyl-3-(2-methyl-4-nitrophenoxy)pyrrolidine

To a stirred solution of 2-methyl-4-nitrophenol (1 g, 6.53 mmol) in THF (10 ml) was added (S)-1-methylpyrrolidin-3-ol (0.661 g, 6.53 mmol), triphenylphosphine (2.57 g, 9.80 mmol) and DEAD (1.551 ml, 9.80 mmol) at 0° C. under inert atmosphere. Then, the resulting reaction mixture was stirred at room temperature for 16 h. The progress of the reaction was monitored by TLC and LCMS., the reaction mixture was diluted with water (50 mL) and extracted with ethyl acetate (2×100 mL). The combined organic extracts were washed with brine (50 mL), dried over anhydrous Na₂SO₄, filtered, and concentrated under reduced pressure to afford crude compound which was purified by flash column chromatography (SiO₂/100-200 mesh; ethyl acetate/n-hexane, 30-50% as eluent) to obtain (R)-1-methyl-3-(2-methyl-4-nitrophenoxy)pyrrolidine (700 mg, 2.58 mmol, 39.5% yield) as a colorless liquid. LCMS (ESI) m/z=236.4 (M+H), t_{R}: 1.30 min, 61.69% (Method-D)

### (R)-3-methyl-4-((1-methylpyrrolidin-3-yl)oxy)aniline

To a degassed solution of (R)-1-methyl-3-(2-methyl-4-nitrophenoxy)pyrrolidine (700 mg, 2.96 mmol) in MeOH (20 ml) was added 10% Pd/C (150 mg) under inert atmosphere. Then, the reaction mixture was stirred under a hydrogen atmosphere using hydrogen bladder pressure at room temperature for 16 h. The progress of the reaction was monitored by TLC and UPLC., the reaction mixture was filtered under a nitrogen atmosphere through a pad of celite to remove the catalyst, and the celite pad was washed with MeOH (2×100 mL). The collected filtrate was concentrated under reduced pressure to get (R)-3-methyl-4-((1-methylpyrrolidin-3-yl)oxy)aniline (500 mg, 1.575 mmol, 53.2% yield) as a black solid, which was taken into the next step without any further purification. LCMS (ESI) m/z=207.3 (M+H), t_{R}: 0.21 min, 65.42% (Method-A).

### (R)—N-(2-bromo-6-chlorophenyl)-4-methoxy-2-((3-methyl-4-((1-methylpyrrolidin-3-yl)oxy)phenyl)amino)pyrimidine-5-carboxamide

To a stirred solution of (R)-3-methyl-4-((1-methylpyrrolidin-3-yl)oxy)aniline (98 mg, 0.477 mmol) in acetic acid (2 mL) was added N-(2-bromo-6-chlorophenyl)-2-chloro-4-methoxypyrimidine-5-carboxamide (150 mg, 0.398 mmol) at room temperature. The resulting reaction mixture was stirred at 25° C. for 16 h. The progress of the reaction was monitored by TLC and LCMS., the solvent was evaporated under reduced pressure to get the crude which was diluted with water and extracted with 10% MeOH-DCM (2×50 mL). The organic layer was washed with saturated sodium bicarbonate (30 mL), dried over Na₂SO₄, filtered, and concentrated under reduced pressure. The resulting crude compound was purified by prep. HPLC (Column: X-select CSH C18, mobile phase A: ammonium acetate in water, mobile phase B: acetonitrile, flowrate:15.0 mL/min, room temperature) to obtain (R)—N-(2-bromo-6-chlorophenyl)-4-methoxy-2-((3-methyl-4-((1-methylpyrrolidin-3-yl)oxy)phenyl)amino)pyrimidine-5-carboxamide (40.56 mg, 0.073 mmol, 40.2% yield) as an off white solid. LCMS (ESI) m/z=546.2 (M+H), t_{R}: 1.41, 98.55% (Method-A). ¹H-NMR (400 MHz, DMSO-d₆): δ 9.96 (s, 1H), 9.55 (s, 1H), 8.73 (s, 1H), 7.74 (d, J=1.20 Hz, 1H), 7.72-7.72 (m, 3H), 7.31 (t, J=8.00 Hz, 1H), 6.88 (d, J=8.80 Hz, 1H), 4.95 (s, 1H), 4.10 (s, 3H), 3.16-2.83 (m, 4H), 2.56 (s, 3H), 2.39-2.35 (m, 1H), 2.17 (s, 3H), 1.96-1.92 (m, 1H)

### Example 70. Synthesis of N-2,6-dibromophenyl-4-methoxy-2-[1-(1-methyl-4-piperidyl)-4-pyrazolylamino]-5-pyrimidinecarboxamide (Compound 212)

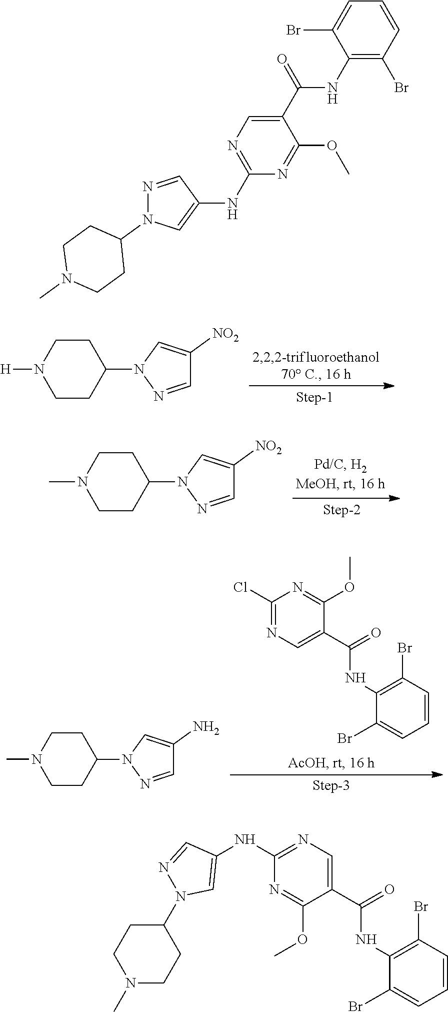

### 1-methyl-4-(4-nitro-1H-pyrazol-1-yl)piperidine

To a stirred solution of 4-(4-nitro-1H-pyrazol-1-yl)piperidine (2 g, 10.19 mmol) in 2,2,2-trifluoroethanol was added paraformaldehyde (1.530 g, 51.0 mmol) at 0° C. under inert atmosphere. The resulting reaction mixture was stirred at 70° C. temperature for 1 h. Then, the reaction mixture was cooled to 0° C. and sodium borohydride (1.157 g, 30.6 mmol) was added lot-wise. The resulting reaction mixture was stirred at room temperature for 16 h. Progress of the reaction was monitored by TLC and LCMS., the reaction mixture was concentrated under reduced pressure. Water was added to the resulting crude residue and extracted with DCM. The organic layer was dried over Na₂SO₄, and concentrated under reduced pressure to give the crude 1-methyl-4-(4-nitro-1H-pyrazol-1-yl)piperidine (800 mg, 3.65 mmol, 35.8% yield) as yellow semi-solid, which was taken into the next step without any further purification. LCMS (ESI) m/z=211.1 (M+H), t_{R}: 0.574 min, 96.52% (Method-A).

### 1-(1-methylpiperidin-4-yl)-1H-pyrazol-4-amine

To a degassed solution of 1-methyl-4-(4-nitro-1H-pyrazol-1-yl)piperidine (800 mg, 3.81 mmol) in MeOH (10 ml) was added 10% Pd/C (300 mg) under inert atmosphere. Then, the reaction mixture was stirred under a hydrogen atmosphere using hydrogen bladder pressure at room temperature for 16 h. The progress of the reaction was monitored by TLC and UPLC., the reaction mixture was filtered under a nitrogen atmosphere through a pad of celite to remove the catalyst, and the celite pad was washed with methanol (2×100 mL). The filtrate was concentrated under reduced pressure to get 1-(1-methylpiperidin-4-yl)-1H-pyrazol-4-amine (650 mg, 3.56 mmol, 94% yield) as a black gummy solid, which was taken into the next step without any further purification. LCMS (ESI) m/z=180.1 (M+H), t_{R}: 0.315 min, 98.81% (Method-D).

### 2,4-dichloro-N-(2,6-dibromophenyl) pyrimidine-5-carboxamide

To a stirred solution of 2,6-dibromoaniline (1, 500 mg, 1.993 mmol) in NMP (2 ml) was added 2,4-dichloropyrimidine-5-carbonyl chloride (Int 2, 421 mg, 1.993 mmol) and the reaction mixture was stirred at rt for 1 h, The progress of the reaction was monitored by TLC and LCMS., reaction mixture was quenched with water and extracted with ethyl acetate, The organic layer was dried over anhydrous Na₂SO₄ and concentrated under reduced pressure to give crude compound which was purified by column chromatography (SiO₂, 60-120 mesh, ethyl acetate/n-hexane 0-20%) to give 2,4-dichloro-N-(2,6-dibromophenyl) pyrimidine-5-carboxamide (3, 300 mg, 0.704 mmol, 35.4% yield) as an off-white solid. LCMS (ESI) m/z=423.7 (M+H), t_{R}: 0.816 min, 75.22% (Method-D).

### N-(2,6-dibromophenyl)-4-methoxy-2-((1-(1-methylpiperidin-4-yl)-1H-pyrazol-4-yl)amino)pyrimidine-5-carboxamide

To a stirred solution of 1-(1-methylpiperidin-4-yl)-1H-pyrazol-4-amine (107 mg, 0.593 mmol) in AcOH (10 ml) was added 2-chloro-N-(2,6-dibromophenyl)-4-methoxypyrimidine-5-carboxamide (250 mg, 0.593 mmol) at room temperature. The resulting reaction mixture was stirred at 25° C. for 16 h. The progress of the reaction was monitored by TLC and LCMS., the reaction mixture was diluted with water and extracted with 10% MeOH-DCM (2×50 mL). The combined organic layer was dried over Na₂SO₄, filtered, and concentrated under reduced pressure. The resulting crude residue was purified by prep. HPLC (X Select C18, 19×250, mobile phase A: 0.1% FA in H₂O, mobile phase B: acetonitrile) to get the pure compound N-(2,6-dibromophenyl)-4-methoxy-2-((1-(1-methylpiperidin-4-yl)-1H-pyrazol-4-yl)amino)pyrimidine-5-carboxamide (37 mg, 0.065 mmol, 11.04% yield) as an off-white solid. LCMS (ESI) m/z=564.2 (M+H), t_{R}: 1.21 min, 98.85% (Method-A). ¹H-NMR (400 MHz, DMSO-d₆): δ 9.78 (s, 1H), 9.39 (s, 1H), 8.73 (s, 1H), 7.95 (s, 1H), 7.74 (d, J=8.00 Hz, 2H), 7.63 (s, 1H), 7.21 (t, J=8.00 Hz, 1H), 4.14-3.99 (m, 4H), 2.86 (d, J=11.60 Hz, 2H), 2.23 (s, 3H), 2.12-2.10 (m, 2H), 2.07-2.01 (m, 4H), 1.99 (s, 4H).

### Example 71. Synthesis of N-(2-bromo-6-chlorophenyl)-2-{4-[4-(dimethylamino)-1-piperidyl]-3-toluidino}-4-methoxy-5-pyrimidinecarboxamide (Compound 213)

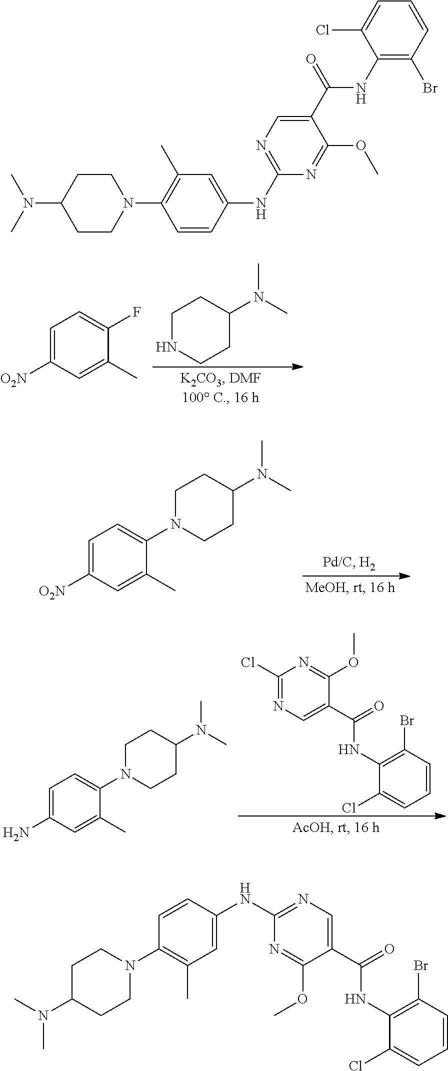

### N,N-dimethyl-1-(2-methyl-4-nitrophenyl)piperidin-4-amine

To a stirred solution of 1-fluoro-2-methyl-4-nitrobenzene (2 g, 12.89 mmol) in DMF (30 ml) were added K₂CO₃ (4.45 g, 32.2 mmol) and N,N-dimethylpiperidin-4-amine (1.517 ml, 12.89 mmol) at 0° C. under inert atmosphere. Then the resulting reaction mixture was stirred at 100° C. for 16 h. The progress of the reaction was monitored by TLC and LCMS., ice-cold water was added to the reaction mixture and the resulting solid was filtered and dried under vacuum to give N,N-dimethyl-1-(2-methyl-4-nitrophenyl)piperidin-4-amine (2.4 g, 9.11 mmol, 70.7% yield) as yellow semi-solid, which was taken into the next step without further purification. LCMS (ESI) m/z=264.1 (M+H), t_{R}: 1.14 min, 69.21% (Method-A).

### 1-(4-amino-2-methylphenyl)-N,N-dimethylpiperidin-4-amine

To a degassed solution of N,N-dimethyl-1-(2-methyl-4-nitrophenyl)piperidin-4-amine (2.4 g, 9.11 mmol) in MeOH (80 ml) was added 10% Pd/C (0.970 g, 9.11 mmol) under nitrogen atmosphere. Then, the reaction mixture was stirred under a hydrogen atmosphere using hydrogen bladder pressure at room temperature for 16 h. The progress of the reaction was monitored by TLC and UPLC., the reaction mixture was filtered under a nitrogen atmosphere through a pad of celite to remove the catalyst, and the celite pad was washed with methanol (2×100 mL). The filtrate was concentrated under reduced pressure to get 1-(4-amino-2-methylphenyl)-N,N-dimethylpiperidin-4-amine (1.8 g, 7.71 mmol, 85% yield) as a black gummy solid which was taken into the next step without any further purification. LCMS (ESI) m/z=234.1 (M+H), t_{R}: 0.80 min, 85.17% (Method-D)

### N-(2-bromo-6-chlorophenyl)-2-((4-(4-(dimethylamino)piperidin-1-yl)-3-methylphenyl)amino)-4-methoxypyrimidine-5-carboxamide

To a stirred solution of 1-(4-amino-2-methylphenyl)-N,N-dimethylpiperidin-4-amine (200 mg, 0.857 mmol) in AcOH (10 ml) was added N-(2-bromo-6-chlorophenyl)-2-chloro-4-methoxypyrimidine-5-carboxamide (323 mg, 0.857 mmol) at room temperature. The resulting reaction mixture was stirred at 25° C. for 16 h. The progress of the reaction was monitored by TLC and LCMS., the reaction mixture was diluted with water and extracted with 10% MeOH-DCM (2×50 mL), washed with aq. NaHCO₃ (50 mL). The combined organic layers was dried over Na₂SO₄, filtered and concentrated under reduced pressure to give the crude compound which was purified by prep. HPLC (X Select C18, 19×250, mobile phase A: 0.1% FA in H₂O, mobile phase B: acetonitrile) to give N-(2-bromo-6-chlorophenyl)-2-((4-(4-(dimethylamino)piperidin-1-yl)-3-methylphenyl)amino)-4-methoxypyrimidine-5-carboxamide (37 mg, 0.064 mmol, 7.52% yield) as an off-white solid. LCMS (ESI) m/z=573.2 (M+H), t_{R}: 1.44 min, 95.01% (Method-A). ¹H-NMR (400 MHz, DMSO-d₆): δ 9.98 (s, 1H), 9.56 (s, 1H), 8.73 (s, 1H), 7.73 (d, J=7.20 Hz, 1H), 7.71-7.54 (m, 3H), 7.31 (t, J=8.00 Hz, 1H), 7.00 (d, J=8.40 Hz, 1H), 4.10 (s, 3H), 3.05 (d, J=11.60 Hz, 2H), 2.68-2.51 (m, 3H), 2.34-2.33 (m, 9H), 1.85-1.83 (m, 2H), 1.55-1.52 (m, 2H)

### Example 72. Synthesis of N-(2-bromo-6-chlorophenyl)-2-{4-[4-(dimethylamino)-1-piperidyl]-3-fluoro-5-toluidino}-4-methoxy-5-pyrimidinecarboxamide (Compound 214)

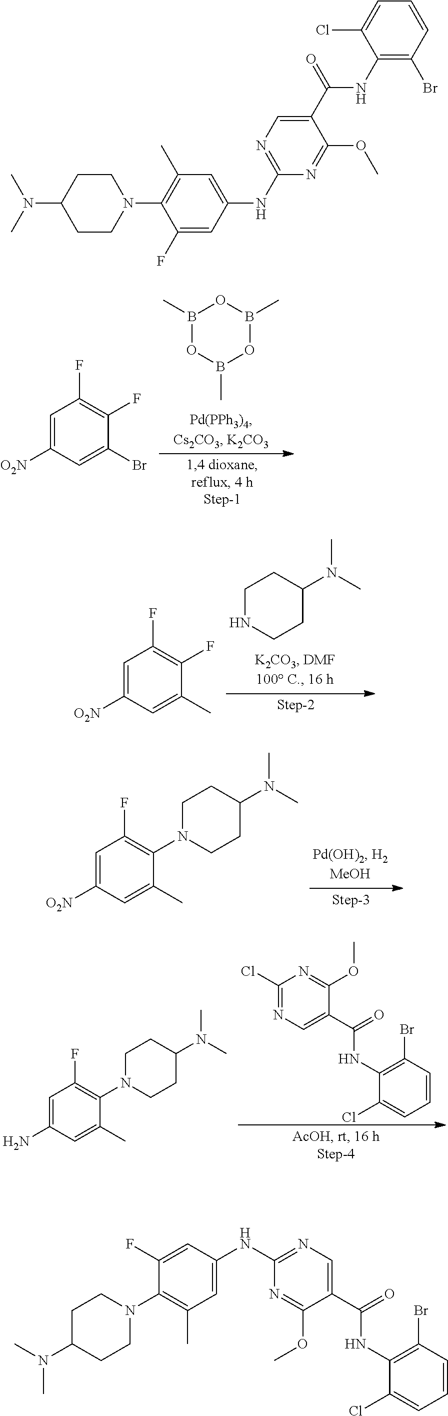

### 1,2-difluoro-3-methyl-5-nitrobenzene

Cs₂CO₃ (0.137 g, 0.420 mmol) and K₂CO₃ (0.116 g, 0.840 mmol) were added to a degassed solution of 1-bromo-2,3-difluoro-5-nitrobenzene (0.100 g, 0.420 mmol) in 1,4-dioxane (25 mL) at room temperature under a nitrogen atmosphere. Pd(PPh₃)₄ (0.049 g, 0.042 mmol) and 2,4,6-trimethyl-1,3,5,2,4,6-trioxatriborinane (0.117 ml, 0.420 mmol) were added. The resulting orange heterogeneous mixture is stirred at reflux for 7 h. The progress of the reaction was monitored by TLC and LCMS. The reaction mixture was cooled to room temperature and concentrated. The residue was diluted with water (30 mL) and extracted with DCM (2×30 mL). The combined extracts was washed with brine (50 mL), dried over sodium sulfate, filtered, and concentrated under reduced pressure. The resulting crude material was purified by flash chromatography (SiO₂/100-200 mesh; ~20-30% EtOAc/hexane) to afford 1,2-difluoro-3-methyl-5-nitrobenzene (0.050 g, 0.289 mmol, 68.7% yield) as a colorless gummy solid. LCMS (ESI) m/z=No mass ionisation. ¹H-NMR (400 MHz, DMSO-d6): δ 8.26 (s, 1H), 8.16 (s, 1H), 2.41 (s, 1H),

### 1-(2-fluoro-6-methyl-4-nitrophenyl)-N,N-dimethylpiperidin-4-amine

1,2-difluoro-3-methyl-5-nitrobenzene (0.500 g, 2.89 mmol), N,N-dimethylpiperidin-4-amine (0.340 ml, 2.89 mmol), and K₂CO₃ (0.998 g, 7.22 mmol) were mixed in DMF (10 ml) at 100° C. under a nitrogen atmosphere. The progress of the reaction was monitored by TLC and UPLC. The reaction mixture was poured into ice-cold water, and a gummy solid was obtained. The solid was collected by filtration and dissolved in ethyl acetate, dried over sodium sulfate and concentrated under reduced pressure to get 1-(2-fluoro-6-methyl-4-nitrophenyl)-N,N-dimethylpiperidin-4-amine (0.400 g, 1.422 mmol, 49.2% yield) as a gummy solid which was taken into the next step without further purification. LCMS (ESI) m/z=282.3 (M+H), t_{R}. 1.739 min, 93.43% (Method B).

### 1-(4-amino-2-fluoro-6-methylphenyl)-N,N-dimethylpiperidin-4-amine

To a degassed solution of 1-(2-fluoro-6-methyl-4-nitrophenyl)-N,N-dimethylpiperidin-4-amine (0.400 g, 1.422 mmol) in methanol (20 mL) was added 10% palladium on carbon (150 mg) under nitrogen atmosphere. The reaction mixture was stirred under a hydrogen atmosphere using hydrogen bladder pressure at room temperature for 16 h. The progress of the reaction was monitored by TLC and UPLC., the reaction mixture was filtered under a nitrogen atmosphere through a pad of celite to remove the catalyst, and the celite pad was washed with methanol (50 mL). The filtrate was concentrated under reduced pressure to give 1-(4-amino-2-fluoro-6-methylphenyl)-N,N-dimethylpiperidin-4-amine (0.300 g, 1.194 mmol, 84% yield) as a light brown gummy solid, which was taken into the next step without further purification.

### N-(2-bromo-6-chlorophenyl)-2-((4-(4-(dimethylamino)piperidin-1-yl)-3-fluoro-5-methylphenyl)amino)-4-methoxypyrimidine-5-carboxamide

To a stirred solution of N-(2-bromo-6-chlorophenyl)-2-chloro-4-methoxypyrimidine-5-carboxamide (0.180 g, 0.477 mmol) in acetic acid (10 mL) was added 1-(4-amino-2-fluoro-6-methylphenyl)-N,N-dimethylpiperidin-4-amine (0.120 g, 0.477 mmol) at room temperature. The resulting reaction mixture was stirred at room temperature for 16 h. The reaction was monitored by TLC. The reaction mixture was diluted with water and extracted with 10% Methanol-DCM (50 mL×2). The combined organic layer was dried over Na₂SO₄, filtered, and concentrated under reduced pressure. The resulting crude residue was purified by prep. HPLC (X SELECT C18, 19×250, mobile phase A: 0.1M FA in H₂O, mobile phase B: acetonitrile) to get N-(2-bromo-6-chlorophenyl)-2-((4-(4-(dimethylamino)piperidin-1-yl)-3-fluoro-5-methylphenyl)amino)-4-methoxypyrimidine-5-carboxamide (0.080 g, 0.130 mmol, 27.3% yield) as an off-white solid. LCMS (ESI) ni/z=592.7 (M+H), t_{R}: 1.72. min, 99.395% (Method-C). ¹H-NMR (400 MHz, DMSO-d6): δ 10.17 (s, 1H), 9.63 (s, 1H), 8.77 (s, 1H), 7.74-7.72 (m, 1H), 7.63-7.60 (m, 2H), 7.36 (s, 1H), 7.31-7.29 (m, 1H), 4.12 (s, 3H), 3.07 (s, 2H), 2.99 (s, 3H), 2.29 (s, 3H), 2.22 (s, 6H), 1.80 (d, J=9.20 Hz, 2H), 1.50 (t, J=4.40 Hz, 2H).

### Example 73. Synthesis of N-(2-bromo-6-chlorophenyl)-2-[3-chloro-4-(1-methyl-4-piperidyl)phenylamino]-4-methoxy-5-pyrimidinecarboxamide (Compound 215)

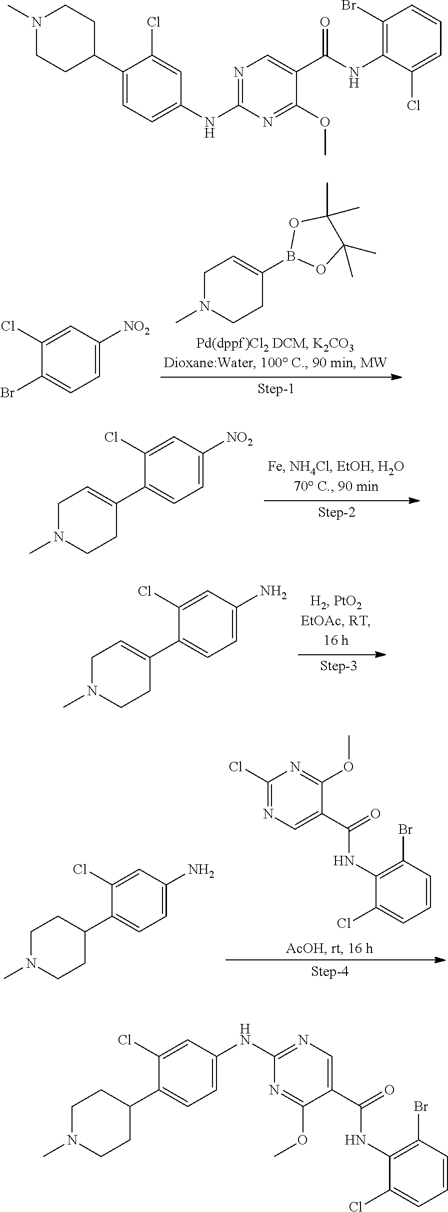

### 4-(2-chloro-4-nitrophenyl)-1-methyl-1,2,3,6-tetrahydropyridine

To a mixture of 1-bromo-2-chloro-4-nitrobenzene (1 g, 4.23 mmol), 1-methyl-4-(4,4,5,5-tetramethyl-1,3,2-dioxaborolan-2-yl)-1,2,3,6-tetrahydropyridine (0.944 g, 4.23 mmol) and potassium carbonate (1.751 g, 12.69 mmol) in dioxane (12 mL) and water (3 mL) was stirred at room temperature. The reaction mixture was degassed with nitrogen for 15 minutes. Pd(dppf)Cl₂ DCM complex (0.345 g, 0.423 mmol) was added to the reaction mixture and reaction mixture was stirred at 100° C. for 1.5 h in microwave irradiation. The progress of the reaction was monitored by TLC after the completion of the reaction. The reaction mixture was filtered through celite bed, the bed was washed with ethyl acetate (50 mL), and the filtrate was concentrated under reduced pressure. The residue was dissolved in ethyl acetate (100 mL) and washed with water (100 mL) and brine (100 mL). The organic extract was dried over anhydrous sodium sulphate, filtered, and concentrated under reduced pressure (bath temperature: 42° C.) to afford a crude compound. The crude product was purified by flash column chromatography (silica-gel, mesh size 60-120) using methanol-DCM (10 to 15%) as an eluent to obtain desired product 4-(2-chloro-4-nitrophenyl)-1-methyl-1,2,3,6-tetrahydropyridine (900 mg, 2.67 mmol, 63.2% yield) as an orange solid. LCMS (ESI) m/z=253.0 &255.0 (M+2), t_{R}: 1.91 min, 75.16% (Method-B).

### 3-chloro-4-(1-methyl-1,2,3,6-tetrahydropyridin-4-yl)aniline

To a stirred solution of 4-(2-chloro-4-nitrophenyl)-1-methyl-1,2,3,6-tetrahydropyridine (500 mg, 1.979 mmol) and iron powder (1105 mg, 19.79 mmol), in ethanol (12 ml) and water (3 ml), ammonium chloride (1058 mg, 19.79 mmol) was added portion-wise in at room temperature. The reaction mixture was allowed to be stirred at 70° C. for 1.5 h. The progress of the reaction was monitored by TLC after the completion of the reaction. The reaction mixture was filtered through a celite bed, the bed was washed with ethyl acetate (100 mL), and the filtrate was concentrated under reduced pressure. The residue was dissolved in ethyl acetate (100 mL) and washed with water (100 mL) and brine (100 mL). The organic extract was dried over anhydrous sodium sulfate, filtered, and concentrated under reduced pressure (bath temperature: 45° C.) to afford crude 3-chloro-4-(1-methyl-1,2,3,6-tetrahydropyridin-4-yl)aniline (650 mg, 1.372 mmol, 69.3% yield) as an orange sticky gum.

### 3-chloro-4-(1-methylpiperidin-4-yl)aniline

To a stirred solution of 3-chloro-4-(1-methyl-1,2,3,6-tetrahydropyridin-4-yl)aniline (550 mg, 2.470 mmol) in ethyl acetate (20 ml), platinum oxide (561 mg, 2.470 mmol) was added portion-wise in reaction at 25° C. The reaction mixture was allowed to be stirred at 25° C. for 16 h. The progress of the reaction was monitored by TLC after the completion of the reaction. The reaction mixture was filtered through a celite bed. The filtrate was collected and concentrated under reduced pressure to afford 3-chloro-4-(1-methylpiperidin-4-yl)aniline (250 mg, 0.612 mmol, 24.78% yield) as a brown sticky gum.

### N-(2-bromo-6-chlorophenyl)-2-((3-chloro-4-(1-methylpiperidin-4-yl)phenyl)amino)-4-methoxypyrimidine-5-carboxamide

To a stirred solution of 3-chloro-4-(1-methylpiperidin-4-yl)aniline (250 mg, 1.112 mmol) in acetic acid (7 ml), N-(2-bromo-6-chlorophenyl)-2-chloro-4-methoxypyrimidine-5-carboxamide (419 mg, 1.112 mmol) was added portion-wise at 25° C. The reaction mixture was stirred at 25° C. for 16 h. The progress of the reaction was monitored by TLC after the completion of the reaction. The reaction mixture was concentrated under reduced pressure, diluted with ice-cold water (100 mL), and extracted with DCM (50 mL×3). The combined organic extract was washed with saturated aqueous sodium bicarbonate solution (100 mL). The organic layer was washed with brine (100 mL), dried over anhydrous sodium sulfate, filtered, and concentrated under reduced pressure (bath temperature: 42° C.) to afford crude compound. The crude compound was purified by Prep-HPLC (FA-method). Pure fractions were collected and lyophilized to afford N-(2-bromo-6-chlorophenyl)-2-((3-chloro-4-(1-methylpiperidin-4-yl)phenyl)amino)-4-methoxypyrimidine-5-carboxamide (36 mg, 0.062 mmol, 5.61% yield) as an off white solid. LCMS (ESI) m/z=566.12 (M+1), t_{R}: 1.39 min, 96.96% (Method-B). ¹H-NMR (400 MHz, DMSO-d6): δ 10.26 (bs, 1H), 9.65-9.64 (d, 4.8 Hz 1H), 8.796-8.780 (d, 6.4 Hz 1H), 8.018-7.994 (s, 9.6 Hz, 1H), 7.742-7.676 (m, 2H), 7.628-7.557 (m, 1H), 7.351-7.295 (m, 2H), 4.128 (s, 1H), 3.552-3.535 (d, 6.8 Hz, 1H), 3.186-3.170 (d, 6.4 Hz, 1H), 2.919-2.790 (m, 2H), 2.219 (s, 3H), 2.035-1.911 (m, 1H), 1.705-1.627 (m, 2H).

### Example 74. Synthesis of N-(2-bromo-6-chlorophenyl)-4-methoxy-2-[4-(4-piperidyloxy)-3-toluidino]-5-pyrimidinecarboxamide (Compound 219)

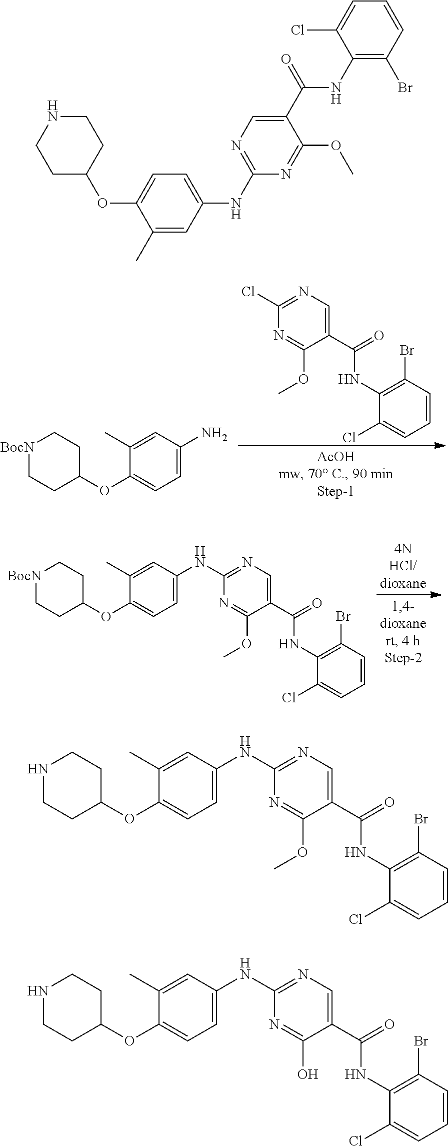 [SMILES: COc1nc(Nc2ccc(OC3CCNCC3)c(C)c2)ncc1C(=O)Nc1c(Cl)cccc1Br ; Cc1cc(N)ccc1OC1CCN(C(=O)OC(C)(C)C)CC1 ; COc1nc(Cl)ncc1C(=O)Nc1c(Cl)cccc1Br ; COc1nc(Nc2ccc(OC3CCN(C(=O)OC(C)(C)C)CC3)c(C)c2)ncc1C(=O)Nc1c(Cl)cccc1Br ; COc1nc(Nc2ccc(OC3CCNCC3)c(C)c2)ncc1C(=O)Nc1c(Cl)cccc1Br ; Cc1cc(Nc2ncc(C(=O)Nc3c(Cl)cccc3Br)c(O)n2)ccc1OC1CCNCC1]

### tert-butyl (R)-3-(4-((5-((2-bromo-6-chlorophenyl)carbamoyl)-4-methoxypyrimidin-2-yl)amino)-2-methylphenoxy)pyrrolidine-1-carboxylate

To a stirred solution of N-(2-bromo-6-chlorophenyl)-2-chloro-4-methoxypyrimidine-5-carboxamide (300 mg, 0.796 mmol) in acetic acid (8 mL) was added tert-butyl 4-(4-amino-2-methylphenoxy)piperidine-1-carboxylate (244 mg, 0.796 mmol) at room temperature. The resulting reaction mixture was subjected to microwave irradiation at 70° C. for 90 min. The reaction was monitored by TLC., the reaction mixture was diluted with water and extracted with 10% Methanol-DCM (50 mL×2). The combined organic layer was dried over Na₂SO₄, filtered and concentrated under reduced pressure to give tert-butyl 4-(4-((5-((2-bromo-6-chlorophenyl)carbamoyl)-4-methoxypyrimidin-2-yl)amino)-2-methylphenoxy)piperidine-1-carboxylate (500 mg, 0.773 mmol, 47% purity) as an off white solid which was used in the next step without any further purification.

### N-(2-bromo-6-chlorophenyl)-4-methoxy-2-((3-methyl-4-(piperidin-4-yloxy)phenyl)amino)pyrimidine-5-carboxamide

To a stirred solution of tert-butyl 4-(4-((5-((2-bromo-6-chlorophenyl)carbamoyl)-4-methoxypyrimidin-2-yl)amino)-2-methylphenoxy)piperidine-1-carboxylate (500 mg, 0.773 mmol) in 1,4-dioxane (5 mL), was added 4N HCl in 1,4-dioxane (2 mL) at 0° C. The resulting reaction mixture was stirred at room temperature for 8 h. The reaction was monitored by TLC., the reaction mixture was concentrated under reduced pressure. The resulting crude residue was purified by prep. HPLC X Select C18, 19×250, mobile phase A: 0.1M FA in H₂O, mobile phase B: acetonitrile) to get N-(2-bromo-6-chlorophenyl)-4-methoxy-2-((3-methyl-4-(piperidin-4-yloxy)phenyl)amino)pyrimidine-5-carboxamide (35 mg, 0.063 mmol, 8.12% yield) as an off-white solid. LCMS (ESI) m/z=546.0 (M+H), t_{R}: 1.429 min, 99.36% (Method-A). ¹H-NMR (400 MHz, DMSO-d₆): δ 9.95 (s, 1H), 9.55 (s, 1H), 8.73 (s, 1H), 7.73 (dd, J=1.20, 8.00 Hz, 1H), 7.61 (dd, J=0.80, 8.20 Hz, 1H), 7.56-7.51 (m, 3H), 7.51-7.29 (m, 1H), 6.97 (d, J=8.80 Hz, 1H), 4.46-4.42 (m, 1H), 4.09 (s, 3H), 3.09-3.04 (m, 2H), 2.18 (s, 3H), 2.18-1.95 (m, 2H), 1.67-1.63 (m, 2H).

### Example 75. Synthesis of N-(2-bromo-6-chlorophenyl)-4-ethoxy-2-[4-(4-piperidyloxy)-3-toluidino]-5-pyrimidinecarboxamide (Compound 221)

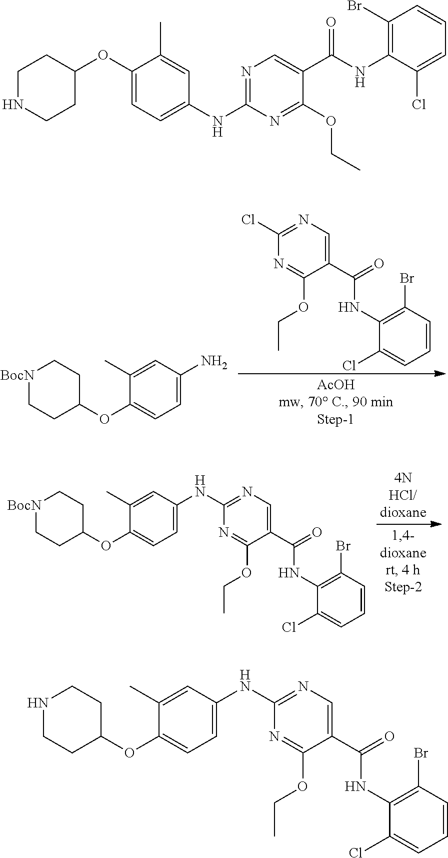 [SMILES: CCOc1nc(Nc2ccc(OC3CCNCC3)c(C)c2)ncc1C(=O)Nc1c(Cl)cccc1Br ; Cc1cc(N)ccc1OC1CCN(C(=O)OC(C)(C)C)CC1 ; CCOc1nc(Cl)ncc1C(=O)Nc1c(Cl)cccc1Br ; CCOc1nc(Nc2ccc(OC3CCN(C(=O)OC(C)(C)C)CC3)c(C)c2)ncc1C(=O)Nc1c(Cl)cccc1Br ; CCOc1nc(Nc2ccc(OC3CCNCC3)c(C)c2)ncc1C(=O)Nc1c(Cl)cccc1Br]

### tert-butyl (R)-3-(4-((5-((2-bromo-6-chlorophenyl)carbamoyl)-4-ethoxypyrimidin-2-yl)amino)-2-methylphenoxy)pyrrolidine-1-carboxylate

To a stirred solution of N-(2-bromo-6-chlorophenyl)-2-chloro-4-ethoxypyrimidine-5-carboxamide (300 mg, 0.767 mmol) in acetic acid (8 mL) was added tert-butyl 4-(4-amino-2-methylphenoxy)piperidine-1-carboxylate (235 mg, 0.767 mmol) at room temperature. The resulting reaction mixture was subjected to microwave irradiation at 70° C. for 90 min. The reaction was monitored by TLC., the reaction mixture was diluted with water and extracted with 10% methanol-DCM (50 mL×2). The combined organic layer was dried over Na₂SO₄, filtered and concentrated under reduced pressure to give tert-butyl 4-(4-((5-((2-bromo-6-chlorophenyl)carbamoyl)-4-ethoxypyrimidin-2-yl)amino)-2-methylphenoxy)piperidine-1-carboxylate (500 mg, 0.767 mmol, 47% purity.

### N-(2-bromo-6-chlorophenyl)-4-ethoxy-2-((3-methyl-4-(piperidin-4-yloxy)phenyl)amino)pyrimidine-5-carboxamide

To a stirred solution of tert-butyl 4-(4-((5-((2-bromo-6-chlorophenyl)carbamoyl)-4-ethoxypyrimidin-2-yl)amino)-2-methylphenoxy)piperidine-1-carboxylate (500 mg, 0.416 mmol) in 1,4-dioxane (5 mL), was added 4N HCl in 1,4-dioxane (2 mL) at 0° C. The resulting reaction mixture was stirred at room temperature for 8 h. The reaction was monitored by TLC., the reaction mixture was concentrated under reduced pressure. The resulting crude residue was purified by prep. HPLC X Select C18, 19×250, mobile phase A: 0.1M FA in H₂O, mobile phase B: acetonitrile) to get N-(2-bromo-6-chlorophenyl)-4-ethoxy-2-((3-methyl-4-(piperidin-4-yloxy)phenyl)amino)pyrimidine-5-carboxamide (70 mg, 0.122 mmol, 29.4% yield) as an off-white solid. LCMS (ESI) m/z=558.0 (M−H), t_{R}: 1.493 min, 95.85% (Method-A). ¹H-NMR (400 MHz, DMSO-d₆): δ 9.92 (s, 1H), 9.45 (s, 1H), 8.72 (s, 1H), 7.73 (dd, J=1.20, 8.20 Hz, 1H), 7.62 (d, J=0.80 Hz, 1H), 7.60 (d, J=1.20 Hz, 1H), 7.57-7.47 (m, 1H), 7.33-7.24 (m, 1H), 6.97 (d, J=8.80 Hz, 1H), 4.60 (q, J=7.20 Hz, 2H), 4.47-4.43 (m, 1H), 3.10-3.05 (m, 2H), 2.83-2.77 (m, 2H), 2.18 (s, 3H), 1.99-1.95 (m, 2H), 1.68-1.64 (m, 2H), 1.45 (t, J=7.20 Hz, 3H),

### Example 76. Synthesis of N-(2-bromo-6-chlorophenyl)-4-ethoxy-2-[4-(1-methyl-4-piperidyloxy)-3-toluidino]-5-pyrimidinecarboxamide (Compound 222)

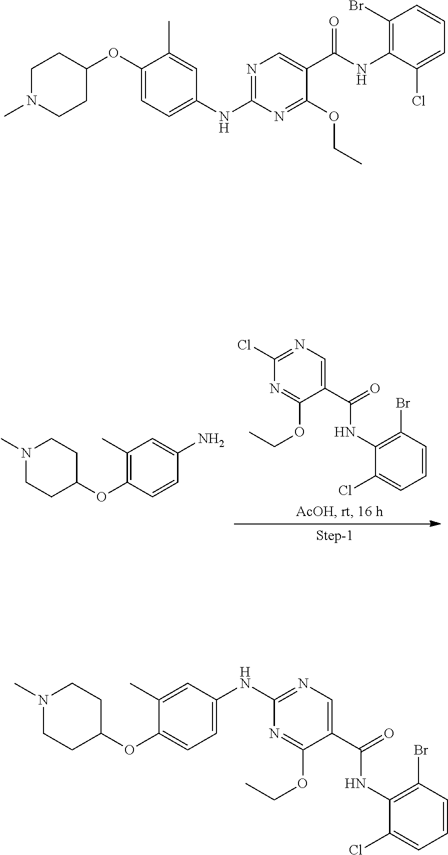 [SMILES: CCOc1nc(Nc2ccc(OC3CCN(C)CC3)c(C)c2)ncc1C(=O)Nc1c(Cl)cccc1Br ; Cc1cc(N)ccc1OC1CCN(C)CC1 ; CCOc1nc(Cl)ncc1C(=O)Nc1c(Cl)cccc1Br ; CCOc1nc(Nc2ccc(OC3CCN(C)CC3)c(C)c2)ncc1C(=O)Nc1c(Cl)cccc1Br]

### N-(2-bromo-6-chlorophenyl)-4-ethoxy-2-((3-methyl-4-((1-methylpiperidin-4-yl)oxy)phenyl)amino)pyrimidine-5-carboxamide

To a stirred solution of 3-methyl-4-((1-methylpiperidin-4-yl)oxy)aniline (100 mg, 0.454 mmol) in acetic acid (3 ml) was added N-(2-bromo-6-chlorophenyl)-2-chloro-4-ethoxypyrimidine-5-carboxamide (177 mg, 0.454 mmol) at room temperature. The resulting reaction mixture was stirred at 25° C. for 16 h. The reaction was monitored by LCMS., the solvent was evaporated under vacuum. The crude was diluted with water and extracted with 10% methanol-DCM (50 mL×2). The organic layer was washed with sat. sodium bicarbonate (30 ml), dried over Na₂SO₄, filtered, and concentrated under reduced pressure. The resulting crude residue was purified by prep. HPLC to obtain N-(2-bromo-6-chlorophenyl)-4-ethoxy-2-((3-methyl-4-((1-methylpiperidin-4-yl)oxy)phenyl)amino)pyrimidine-5-carboxamideide (43 mg, 0.073 mmol, 15.2% yield) as an off white solid. LCMS (ESI) m/z=574.0 (M+H), t_{R}: 1.46 min, 95.12% (Method-A). ¹H-NMR (400 MHz, DMSO-d₆): δ 9.91 (s, 1H), 9.45 (s, 1H), 8.72 (s, 1H), 7.74 (d, J=1.20 Hz, 1H), 7.72 (d, J=1.20 Hz, 1H), 7.62 (t, J=6.80 Hz, 1H), 7.60-7.54 (m, 1H), 7.46-7.31 (m, 1H), 6.95 (d, J=8.80 Hz, 1H), 4.60 (q, J=7.20 Hz, 2H), 4.31 (m, 1H), 2.51-2.50 (m, 2H), 2.22-2.17 (m, 8H), 0.93 (m, 4H), 1.70-1.67 (m, 2H), 1.45 (t, J=7.20 Hz, 3H).

### Example 77. Synthesis of N-(3-hydroxy-2,6-xylyl)-4-methoxy-2-(1-methyl-4-pyrazolylamino)-5-pyrimidinecarboxamide (Compound 224)

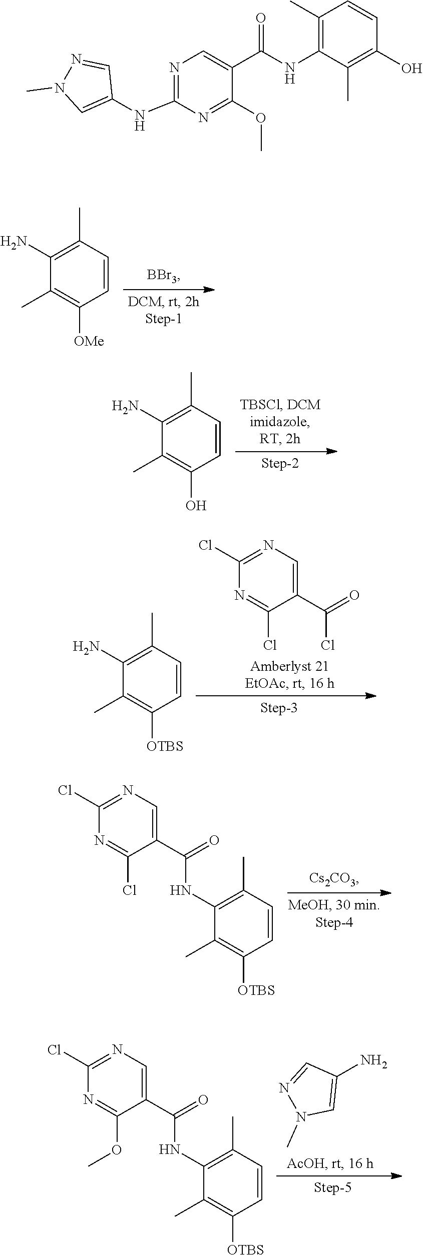

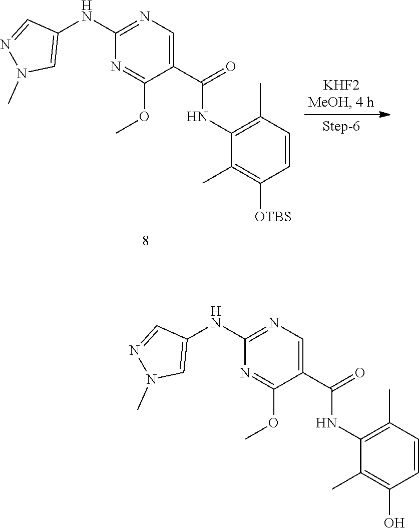 [SMILES: COc1nc(Nc2cnn(C)c2)ncc1C(=O)Nc1c(C)ccc(O[Si](C)(C)C(C)(C)C)c1C ; COc1nc(Nc2cnn(C)c2)ncc1C(=O)Nc1c(C)ccc(O)c1C]

### 3-amino-2,4-dimethylphenol

To a stirred solution of 3-methoxy-2,6-dimethylaniline (2 g, 13.23 mmol) in DCM (70 ml) BBr3 (26.5 ml, 26.5 mmol) was added at 0° C. After 10 min at this temperature, the reaction mixture was allowed to be stirred at room temperature for 2 h. The reaction was monitored by LCMS. The reaction mixture was quenched with sodium bicarbonate solution and then diluted with ethyl acetate (500 ml) and washed with water (200 ml). The combined organic extract was washed with brine (250 mL), dried over anhydrous sodium sulfate, filtered, and concentrated under reduced pressure (bath temperature: 45° C.) to afford 3-amino-2,4-dimethylphenol (1.8 g, 12.11 mmol, 92% yield). This crude product was taken as such for the subsequent step. LCMS (ESI) m/z=138.1 (M+1), t_{R}: 0.55 min, Purity-92.35% (Method-A).

### 3-((tert-butyldimethylsilyl)oxy)-2,6-dimethylaniline

To a stirred solution of 3-amino-2,4-dimethylphenol (1.8 g, 13.12 mmol) in DCM (30 ml), imidazole (2.233 g, 32.8 mmol) and TBSCl (2.97 g, 19.68 mmol) were added at 0° C. The Reaction mixture was stirred at room temperature for 2 h. The reaction was monitored by LCMS. The reaction mixture was quenched with sodium bicarbonate solution and then extracted with ethyl acetate (500 ml). The combined organic extract was washed with brine (250 mL), dried over anhydrous sodium sulfate, filtered, and concentrated under reduced pressure (bath temperature: 45° C.) to afford crude compound. The crude product was purified by combi flash column chromatography (silica-gel, mesh size-230-400) using ethyl acetate/hexane (20-25%) as an eluent to obtain desired product 3-((tert-butyldimethylsilyl)oxy)-2,6-dimethylaniline (2.8 g, 10.69 mmol, 81% yield) as a light brown liquid. LCMS (ESI) im/z=252.4 (M+1), t_{R}: 2.28 min, Purity-96.18% (Method-A).

### N-(3-((tert-butyldimethylsilyl)oxy)-2,6-dimethylphenyl)-2,4-dichloropyrimidine-5-carboxamide

To a stirred solution of 3-((tert-butyldimethylsilyl)oxy)-2,6-dimethylaniline (400 mg, 1.591 mmol) and 2,4-dichloropyrimidine-5-carbonyl chloride (336 mg, 1.591 mmol) in ethyl acetate (150 ml), amberlyst A/26 (141 mg, 0.398 mmol) was added to the reaction mixture. The reaction mixture was stirred at room temperature for 16 h. The reaction was monitored by LCMS. The Reaction mixture was diluted with ice-cold water (100 mL) and extracted with ethyl acetate (100 mL×3). The combined organic extract was washed with (100 ml 1.5 N) aqueous sodium bicarbonate (50 ml). The organic layer was washed with brine (100 mL). The organic extract was dried over anhydrous sodium sulfate, filtered, and concentrated under reduced pressure (bath temperature: 45° C.) to afford crude compound N-(3-((tert-butyldimethylsilyl)oxy)-2,6-dimethylphenyl)-2,4-dichloropyrimidine-5-carboxamide as an brown solid (630 mg).

### N-(3-((tert-butyldimethylsilyl)oxy)-2,6-dimethylphenyl)-2-chloro-4-methoxypyrimidine-5-carboxamide

To a stirred solution of N-(3-((tert-butyldimethylsilyl)oxy)-2,6-dimethylphenyl)-2,4-dichloropyrimidine-5-carboxamide (600 mg, 1.407 mmol) in methanol (6 mL), cesium carbonate (367 mg, 1.126 mmol) was added at 0° C. The reaction mixture was stirred at room temperature for 30 min. The reaction was monitored by LCMS. The reaction mixture was concentrated under reduced pressure, and 20 ml water was added and the resulting solid filtered on a sintered funnel. The solid was dried under vacuum to afford N-(3-((tert-butyldimethylsilyl)oxy)-2,6-dimethylphenyl)-2-chloro-4-methoxypyrimidine-5-carboxamide (370 mg, 0.763 mmol, 54.2% yield) as an off white solid. LCMS (ESI) m/z=422 (M+1), t_{R}: 2.46 min (Method-A).

### N-(3-((tert-butyldimethylsilyl)oxy)-2,6-dimethylphenyl)-4-methoxy-2-((1-methyl-1H-pyrazol-4-yl)amino)pyrimidine-5-carboxamide

To a stirred solution of 1-methyl-1H-pyrazol-4-amine (99 mg, 1.024 mmol) in acetic acid (5 mL), N-(3-((tert-butyldimethylsilyl)oxy)-2,6-dimethylphenyl)-2-chloro-4-methoxypyrimidine-5-carboxamide (360 mg, 0.853 mmol) was added at 25° C. The reaction mixture was stirred at room temperature for 16 h. The reaction was monitored by LCMS. The reaction mixture was concentrated under vacuum. The resulting crude gum was dissolved in DCM and quenched in aq. sodium bicarbonate solution and this mixture was extracted with DCM. The organic layers were dried over sodium sulfate and concentrated under vacuum to afford crude compound N-(3-((tert-butyldimethylsilyl)oxy)-2,6-dimethylphenyl)-4-methoxy-2-((1-methyl-1H-pyrazol-4-yl)amino)pyrimidine-5-carboxamide (429 mg, 0.415 mmol, 48.6% yield) as an brown solid.

### N-(3-hydroxy-2,6-dimethylphenyl)-4-methoxy-2-((1-methyl-1H-pyrazol-4-yl)amino)pyrimidine-5-carboxamide

To a stirred solution of N-(3-((tert-butyldimethylsilyl)oxy)-2,6-dimethylphenyl)-2-chloro-4-methoxypyrimidine-5-carboxamide (420 mg, 0.995 mmol) in methanol (5 mL), potassium fluoride (155 mg, 1.991 mmol) was added at 0° C. The mixture was stirred at room temperature for 4 h. The reaction was monitored by LCMS. The reaction mixture was concentrated under vacuum. The residual crude gum was dissolved in 50 ml water and extracted with DCM. The organic layers were dried over sodium sulfate and concentrated under vacuum to afford crude compound. The crude compound was purified by prep-HPLC to afford N-(3-hydroxy-2,6-dimethylphenyl)-4-methoxy-2-((1-methyl-1H-pyrazol-4-yl)amino)pyrimidine-5-carboxamide (118 mg, 0.307 mmol, 30.9% yield) as an off white solid. LCMS (ESI) m/z=369.3 (M+1), t_{R}: 1.46 min, Purity-95.99% (Method-B). ¹H-NMR (400 MHz, DMSO-d₆): δ 10.03 (bs, 1H), 9.13-9.07 (d, J=8 Hz, 2H), 8.66 (bs, 1H), 7.89 (bs, 1H), 7.58 (bs, 1H), 6.89-6.87 (d, J=8 Hz, 1H), 6.69-6.67 (d, J=8 Hz, 1H), 4.13 (s, 3H), 3.83 (s, 3H), 2.07 (s, 3H), 1.99 (s, 3H).

### Example 78. Synthesis of N-(2-bromo-6-ethynylphenyl)-4-methoxy-2-((3-methyl-4-(piperidin-4-yl)phenyl)amino)pyrimidine-5-carboxamide (Compound 184)

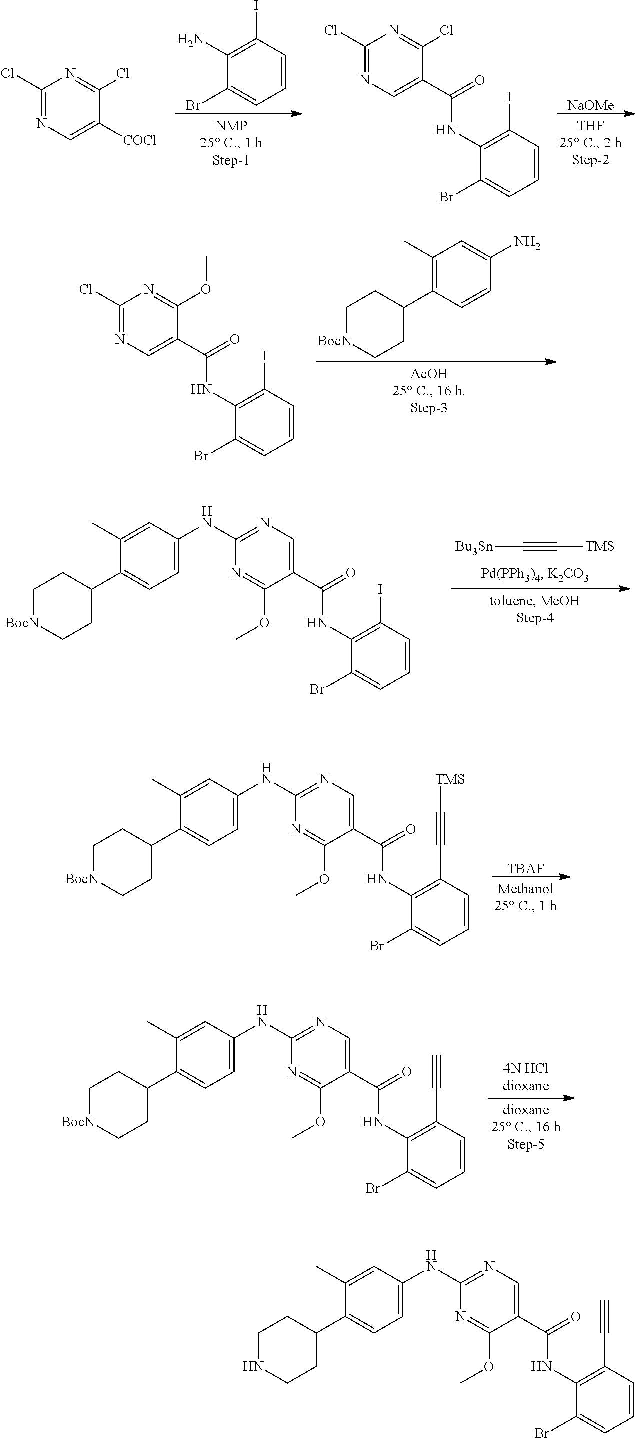

### N-(2-bromo-6-iodophenyl)-2,4-dichloropyrimidine-5-carboxamide

To a stirred solution of 2-bromo-6-iodoaniline (500 mg, 1.678 mmol) in NMP (2 ml) was added 2,4-dichloropyrimidine-5-carbonyl chloride (355 mg, 1.678 mmol) and the resulting mixture was stirred at room temperature for 1 h. The progress of the reaction was monitored by TLC and LCMS. Water was added to the reaction mixture which was then extracted with ethyl acetate (2×100 mL). The combined organic layers were dried over anhydrous Na₂SO₄ and concentrated under reduced pressure. The crude residue was purified by silica gel column chromatography (SiO₂/100-200 mesh; ethyl acetate/n-hexane, 50-60% as an eluent) to get N-(2-bromo-6-iodophenyl)-2,4-dichloropyrimidine-5-carboxamide (400 mg, 0.846 mmol, 50.4% yield) as an off-white solid. LCMS (ESI) m/z=471.9 (M+2), t_{R}. 1.77 min, 69.19% (Method A).

### N-(2-bromo-6-iodophenyl)-2-chloro-4-methoxypyrimidine-5-carboxamide

To a stirred solution of N-(2-bromo-6-iodophenyl)-2,4-dichloropyrimidine-5-carboxamide (500 mg, 1.057 mmol) in THE (20 ml) was added sodium methanolate (57.1 mg, 1.057 mmol) and the resulting mixture was stirred at room temperature for 2 h. The progress of the reaction was monitored by TLC and LCMS. Water was added to the reaction mixture which was then extracted with ethyl acetate (2×100 mL). The combined organic layers were dried over anhydrous Na₂SO₄ and concentrated under reduced pressure. The residue was purified by silica gel column chromatography (SiO₂/100-200 mesh; ethyl acetate/n-hexane, 50-60% as an eluent) to get N-(2-bromo-6-iodophenyl)-2-chloro-4-methoxypyrimidine-5-carboxamide (300 mg, 0.640 mmol, 60.6% yield) as an off-white solid. LCMS (ESI) im/z=468.0 (M+H), t_{R}. 1.87 min, 69.18% (Method D).

### tert-butyl 4-(4-((5-((2-bromo-6-iodophenyl)carbamoyl)-4-methoxypyrimidin-2-yl)amino)-2-methylphenyl)piperidine-1-carboxylate

To a stirred solution of N-(2-bromo-6-iodophenyl)-2-chloro-4-methoxypyrimidine-5-carboxamide (300 mg, 0.640 mmol) in acetic acid (2 ml) was added tert-butyl 4-(4-amino-2-methylphenyl)piperidine-1-carboxylate (186 mg, 0.640 mmol). The mixture was stirred at room temperature for 16 h. The progress of the reaction was monitored by TLC and LCMS. Water was added to the reaction mixture which was then extracted with DCM (2×100 mL). The combined organic layers were dried over anhydrous Na₂SO₄ and concentrated under reduced pressure. The residue was purified by silica gel column chromatography (SiO₂/100-200 mesh; ethyl acetate/n-hexane, 50-60% as an eluent) to get tert-butyl 4-(4-((5-((2-bromo-6-iodophenyl)carbamoyl)-4-methoxypyrimidin-2-yl)amino)-2-methylphenyl)piperidine-1-carboxylate (250 mg, 0.346 mmol, 54.0% yield) as an off white solid.

### tert-butyl 4-(4-((5-((2-bromo-6-((trimethylsilyl)ethynyl)phenyl)carbamoyl)-4-methoxypyrimidin-2-yl)amino)-2-methylphenyl)piperidine-1-carboxylate

A solution of tert-butyl 4-(4-((5-((2-bromo-6-iodophenyl)carbamoyl)-4-methoxypyrimidin-2-yl)amino)-2-methylphenyl)piperidine-1-carboxylate (250 mg, 0.346 mmol) and trimethyl((tributylstannyl)ethynyl)silane (0.230 ml, 0.415 mmol) in toluene (5 ml) was degassed for 15 min with N₂. Then Pd(PPh₃)₄ (40.0 mg, 0.035 mmol) was added and the reaction stirred at 100° C. for 16 h. The progress of the reaction was monitored by TLC and LCMS. Water was added to the reaction mixture which was then extracted with ethyl acetate (2×50 mL). The combined organic layers were dried over anhydrous Na₂SO₄ and concentrated under reduced pressure. The residue was purified by silica gel column chromatography (SiO₂/100-200 mesh; ethyl acetate/n-hexane, 30-60% as eluent) to get tert-butyl 4-(4-((5-((2-bromo-6-((trimethylsilyl)ethynyl)phenyl)carbamoyl)-4-methoxypyrimidin-2-yl)amino)-2-methylphenyl)piperidine-1-carboxylate (150 mg, 0.217 mmol, 62.6% yield) as a red gum. LCMS (ESI) m/z=692.0 (M+H), t_{R}. 2.60 min, 72.67% (Method D).

### tert-butyl 4-(4-((5-((2-bromo-6-ethynylphenyl)carbamoyl)-4-methoxypyrimidin-2-yl)amino)-2-methylphenyl)piperidine-1-carboxylate

To a stirred solution of tert-butyl 4-(4-((5-((2-bromo-6-((trimethylsilyl)ethynyl)phenyl)carbamoyl)-4-methoxypyrimidin-2-yl)amino)-2-methylphenyl)piperidine-1-carboxylate (150 mg, 0.156 mmol) in THF (5 ml) was added TBAF (1M THF, 0.312 ml, 0.312 mmol) at 25° C. under inert atmosphere. Then, the reaction was stirred at 25° C. for 1 h. The progress of the reaction was monitored by TLC and LCMS. Water was added to the reaction mixture which was then extracted with ethyl acetate (2×50 mL). The combined organic layers were dried over anhydrous Na₂SO₄, concentrated under reduced pressure. The residue was purified by silica gel column chromatography (SiO₂/100-200 mesh; ethyl acetate/n-hexane, 30-60% as an eluent) to get tert-butyl 4-(4-((5-((2-bromo-6-ethynylphenyl)carbamoyl)-4-methoxypyrimidin-2-yl)amino)-2-methylphenyl)piperidine-1-carboxylate (60 mg, 0.0966 mmol,) as a gummy solid. LCMS (ESI) m/z=621.6 (M+H), t_{R}: 2.379 min, 96.53% (Method-A).

### N-(2-bromo-6-ethynylphenyl)-4-methoxy-2-((3-methyl-4-(piperidin-4-yl)phenyl)amino)pyrimidine-5-carboxamide

To a stirred solution of tert-butyl 4-(4-((5-((2-bromo-6-ethynylphenyl)carbamoyl)-4-methoxypyrimidin-2-yl)amino)-2-methylphenyl)piperidine-1-carboxylate (60 mg, 0.097 mmol) in DCM (2 ml) was added 4M HCl in dioxane (1 ml, 0.222 mmol) under inert atmosphere at 0° C., and the resulting mixture was stirred at room temperature for 2 h. The progress of the reaction was monitored by TLC and LCMS. The reaction mixture was quenched with sodium bicarbonate and extracted with DCM (2×50 mL). The organic layer was washed with brine, dried over anhydrous sodium sulfate and concentrated under reduced pressure. The crude was purified by prep-HPLC (Column: X-Bridge C₈, mobile phase A: 10 mM ammonium bicarbonate, mobile phase B: acetonitrile, flow rate-15.0 mL/min, room temperature) to get the pure compound N-(2-bromo-6-ethynylphenyl)-4-methoxy-2-((3-methyl-4-(piperidin-4-yl)phenyl)amino)pyrimidine-5-carboxamide (6 mg, 0.0084 mmol, 6% yield) as a pale yellow solid. LCMS (ESI) m/z=520.2 (M+2), t_{R}. 1.60 min, 94.94% (Method D). ¹H-NMR (400 MHz, DMSO-d₆,): δ 10.07 (s, 1H), 9.57 (s, 1H), 8.86-8.83 (m, 1H), 8.76-8.56 (m, 2H), 7.78 (dd, J=1.60, 8.20 Hz, 1H), 7.62-7.57 (m, 3H), 7.30 (t, J=8.00 Hz, 1H), 7.31-7.28 (m, 1H), 4.37 (s, 1H), 4.22-4.18 (m, 1H), 4.12 (s, 3H), 3.39-3.35 (m, 3H), 3.08-3.00 (m, 4H), 2.34-2.33 (m, 4H).

### Example 79. N-(2,6-dichlorophenyl)-4-methoxy-2-((4-(4-methyl-7-oxo-1,4-diazepan-1-yl)phenyl)amino)pyrimidine-5-carboxamide (Compound 185)

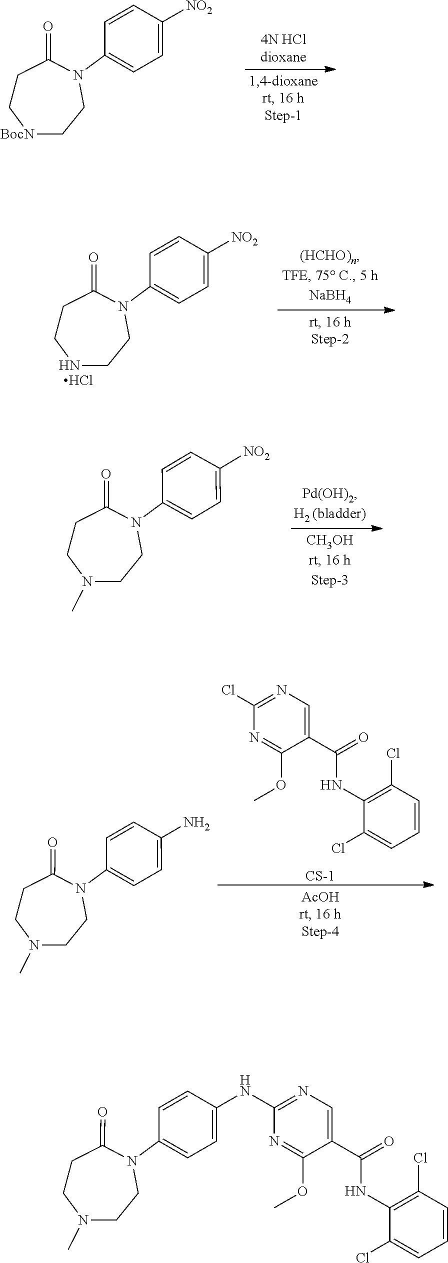

### 4-(4-nitrophenyl)-1,4-diazepan-5-one hydrochloride

To a stirred solution of tert-butyl 4-(4-nitrophenyl)-5-oxo-1,4-diazepane-1-carboxylate (415 mg, 1.237 mmol) in 1,4-dioxane (10 ml) was added 4M HCl in 1,4-dioxane (2.53 mL, 10.12 mmol) at 25° C. and the resulting mixture was stirred for 3 h at 25° C. The progress of the reaction was monitored by TLC and LCMS. The reaction mixture was concentrated under reduced pressure, and the crude solid was washed with MTBE (20 mL) and dried to get 4-(4-nitrophenyl)-1,4-diazepan-5-one (290 mg, 1.073 mmol, 87% yield) as an off white solid. LCMS (ESI) m/z=236.1 (M−H), t_{R}=0.76 min, 87.37% (Method-A).

### 1-((1-methylpyrrolidin-3-yl)methyl)-3-nitro-1H-pyrazole

To a stirred solution of 4-(4-nitrophenyl)-1,4-diazepan-5-one (150 mg, 0.638 mmol) in 2,2,2-trifluroethanol (8 mL) was added paraformaldehyde (96 mg, 3.19 mmol) and the resulting mixture was stirred for 5 h at 75° C. Sodium borohydride (72.4 mg, 1.913 mmol) was added to the reaction mixture at 0° C. which was then stirred for 16 h at 25° C. The progress of the reaction was monitored by TLC and LCMS. The reaction mixture was concentrated under reduced pressure to afford a crude product. The crude residue was diluted with water and extracted with 10% methanol-DCM (50 mL×2). The combined organic layer was dried over Na₂SO₄, filtered, and concentrated under reduced pressure to afford (1-methyl-4-(4-nitrophenyl)-1,4-diazepan-5-one (150 mg, 0.560 mmol, 88% yield) as an off-white gummy solid. LCMS (ESI) m/z=250.2 (M+H), t_{R}=1.04 min, 93.06% (Method-A).

### 4-(4-aminophenyl)-1-methyl-1,4-diazepan-5-one

To a degassed solution of (1-methyl-4-(4-nitrophenyl)-1,4-diazepan-5-one (150 mg, 0.602 mmol) in methanol (10 ml) was added 10% palladium on carbon (50 mg) under nitrogen atmosphere. The reaction mixture was stirred under a hydrogen atmosphere using hydrogen bladder pressure at room temperature for 16 h. The progress of the reaction was monitored by TLC and LCMS. The reaction mixture was filtered under a nitrogen atmosphere through a pad of celite to remove the catalyst, and the celite pad was washed with methanol (50 mL). The filtrate was concentrated under reduced pressure to get 4-(4-aminophenyl)-1-methyl-1,4-diazepan-5-one (100 mg, 0.410 mmol, 68.2% yield) as a brown gum, which was taken into the next step without further purification. LCMS (ESI) m/z=220.1 (M+H), t_{R}=0.57 min, 90.69% (Method-D).

### N-(2,6-dichlorophenyl)-4-methoxy-2-((4-(4-methyl-7-oxo-1,4-diazepan-1-yl)phenyl)amino)pyrimidine-5-carboxamide

To a stirred solution of 2-chloro-N-(2,6-dichlorophenyl)-4-methoxypyrimidine-5-carboxamide (100 mg, 0.301 mmol) in acetic acid (2 mL) was added 4-(4-aminophenyl)-1-methyl-1,4-diazepan-5-one (65.9 mg, 0.301 mmol) at 25° C. under nitrogen atmosphere. The resulting reaction mixture was stirred at room temperature for 16 h. The progress of the reaction was monitored by TLC and LCMS. The reaction mixture was diluted with water and extracted with 10% methanol-DCM (50 mL×2). The combined organic layer was dried over Na₂SO₄, filtered, and concentrated under reduced pressure. The resulting crude residue was purified by prep. HPLC to get N-(2,6-dichlorophenyl)-4-methoxy-2-((4-(4-methyl-7-oxo-1,4-diazepan-1-yl)phenyl)amino)pyrimidine-5-carboxamide (30.56 mg, 0.059 mmol, 19.52% yield) as an off-white solid. LCMS (ESI) m/z=516.4 (M+2H), t_{R}=1.216 min, 99.51% (Method-A). ¹H-NMR (400 MHz, DMSO-d₆): δ 10.20 (s, 1H), 9.59 (s, 1H), 8.77 (s, 1H), 7.77 (d, J=8.80 Hz, 2H), 7.58 (d, J=8.00 Hz, 2H), 7.39 (t, J=8.40 Hz, 1H), 7.17 (dd, J=2.00, 6.80 Hz, 2H), 4.12 (s, 3H), 3.80-3.78 (m, 2H), 2.72-2.70 (m, 2H), 2.64-2.57 (m, 4H), 2.29 (s, 3H).

### Example 80. Synthesis of (N-(2-bromo-6-chlorophenyl)-4-methoxy-2-((3-methyl-4-(1-methyl-1H-pyrrol-3-yl)phenyl)amino)pyrimidine-5-carboxamide (Compound 191)

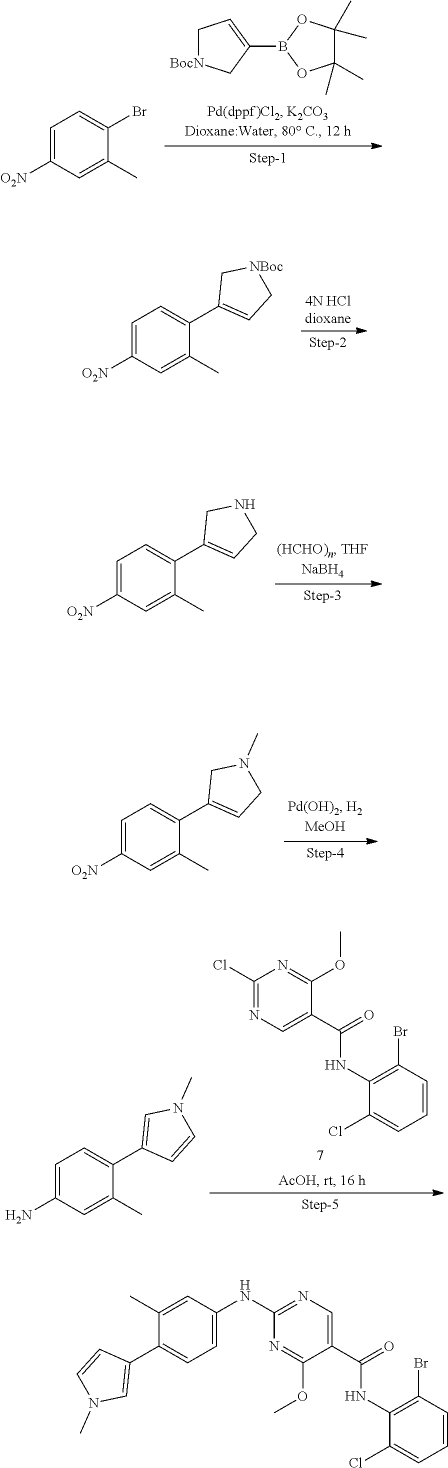

### tert-butyl 3-(2-methyl-4-nitrophenyl)-2,5-dihydro-1H-pyrrole-1-carboxylate

To a stirred solution of 1-bromo-2-methyl-4-nitrobenzene (4 g, 18.52 mmol) in 1,4-dioxane (24 ml) and water (16 ml), K₂CO₃ (3.33 g, 55.5 mmol) and tert-butyl 3-(4,4,5,5-tetramethyl-1,3,2-dioxaborolan-2-yl)-2,5-dihydro-1H-pyrrole-1-carboxylate (5.47 g, 18.52 mmol) were added and then the mixture was degassed with nitrogen for 15 min. To the reaction mixture Pd(dppf)Cl₂ (0.677 g, 0.926 mmol) was added and then the mixture was stirred at 80° C. for 16 h. The progress of the reaction was monitored by TLC and LCMS. The reaction mixture was diluted with water (100 mL) and extracted with 10% methanol-DCM (250 mL×3). The combined organic extract was washed with brine (150 mL), dried over anhydrous sodium sulfate, filtered, and concentrated under reduced pressure (bath temperature: 45° C.) to afford crude product. The crude product was purified by combi flash column chromatography (silica-gel, mesh size-60-120) using ethyl acetate/hexane (25-30%) as an eluent to obtain a tert-butyl 3-(2-methyl-4-nitrophenyl)-2,5-dihydro-1H-pyrrole-1-carboxylate (4 g, 12.97 mmol, 70.1% yield) as a brown viscous liquid. LCMS (ESI) m/z=205.0 (M−100).

### 3-(2-methyl-4-nitrophenyl)-2,5-dihydro-1H-pyrrole

To a stirred solution of tert-butyl 3-(2-methyl-4-nitrophenyl)-2,5-dihydro-1H-pyrrole-1-carboxylate (1.3 g, 4.27 mmol) in DCM was added 4M HCl in dioxane (10.68 ml, 42.7 mmol) at 0° C. The reaction mixture was stirred at room temperature for 2 h. Progress of the reaction was monitored by TLC. The reaction mixture was concentrated under reduced pressure to get crude 3-(2-methyl-4-nitrophenyl)-2,5-dihydro-1H-pyrrole (0.8 g, 3.84 mmol, 90% yield). LCMS (ESI) m/z=205.1 (M+1).

### 1-methyl-3-(2-methyl-4-nitrophenyl)-2,5-dihydro-1H-pyrrole

To a stirred solution of 3-(2-methyl-4-nitrophenyl)-2,5-dihydro-1H-pyrrole (1.65 g, 8.08 mmol) in TFE (15 mL) at 0° C., paraformaldehyde (1.213 g, 40.4 mmol) was added and the resulting mixture stirred at 75° C. for 2 h. The reaction mixture was cooled at 0° C. and sodium borohydride (0.703 g, 18.58 mmol) was added portion-wise. The reaction mixture was allowed to stir at 75° C. for 12 h. The progress of the reaction was monitored by TLC and LCMS. The Reaction mixture was diluted with water (100 mL) and extracted with 10% methanol-DCM (100 mL×3), dried over anhydrous sodium sulfate, filtered, and concentrated under reduced pressure (bath temperature: 45° C.) to afford crude product 1-methyl-3-(2-methyl-4-nitrophenyl)-2,5-dihydro-1H-pyrrole (1.3 g, 4.77 mmol, 59.0% yield). LCMS (ESI) m/z=219.65 (M+1).

### 3-methyl-4-(1-methylpyrrolidin-3-yl)aniline

To a degassed solution of 1-methyl-3-(2-methyl-4-nitrophenyl)-2,5-dihydro-1H-pyrrole (1.3 g, 5.96 mmol) in MeOH (35.00 ml) and THE (15.0 ml) was added 10% Pd—C(0.198 g, 1.191 mmol) portion wise under nitrogen atmosphere. The reaction mixture was stirred for 16 h under a hydrogen atmosphere using a bladder (1 atm) at room temperature. The progress of the reaction was monitored by TLC and LCMS. The reaction mixture was filtered through a small pad of celite which was washed with MeOH. The combined organic extract was concentrated to afford 3-methyl-4-(1-methylpyrrolidin-3-yl)aniline (1.12 g, 5.89 mmol, 99% yield). LCMS (ESI) m/z=187.2 (M+1).

### N-(2-bromo-6-chlorophenyl)-4-methoxy-2-((3-methyl-4-(1-methyl-1H-pyrrol-3-yl)phenyl)amino)pyrimidine-5-carboxamide

3-methyl-4-(1-methyl-1H-pyrrol-3-yl)aniline (89 mg, 0.477 mmol) and N-(2-bromo-6-chlorophenyl)-2-chloro-4-methoxypyrimidine-5-carboxamide (150 mg, 0.398 mmol) were mixed in acetic acid (2 mL). The reaction mixture was stirred at room temperature for 12 h. The progress of the reaction was monitored by TLC and LCMS. Acetic acid was evaporated under reduced pressure, and the resulting crude compound was purified by prep-HPLC. The fraction was lyophilized to get N-(2-bromo-6-chlorophenyl)-4-methoxy-2-((3-methyl-4-(1-methyl-1H-pyrrol-3-yl)phenyl)amino)pyrimidine-5-carboxamide (55 mg, 0.103 mmol, 26.0% yield). LCMS (ESI) m/z=527.8 (M+1). ¹H-NMR (400 MHz, DMSO-d₆): δ 10.07 (s, 1H), 9.58 (s, 1H), 8.76 (s, 1H), 7.74 (d, J=1.20 Hz, 1H), 7.62-7.56 (m, 3H), 7.33-7.27 (m, 2H), 6.89 (d, J=2.00 Hz, 1H), 6.78 (q, J=2.40 Hz, 1H), 6.22 (t, J=2.40 Hz, 1H), 4.13 (s, 3H), 3.66 (s, 3H), 2.39 (s, 3H),

### Example 81. Synthesis of (R)—N-(2-bromo-6-chlorophenyl)-4-methoxy-2-((3-methyl-4-(1-methylpiperidin-3-yl)phenyl)amino)pyrimidine-5-carboxamide (Compound 196) and (S)—N-(2-bromo-6-chlorophenyl)-4-methoxy-2-((3-methyl-4-(1-methylpiperidin-3-yl)phenyl)amino)pyrimidine-5-carboxamide (Compound 197)

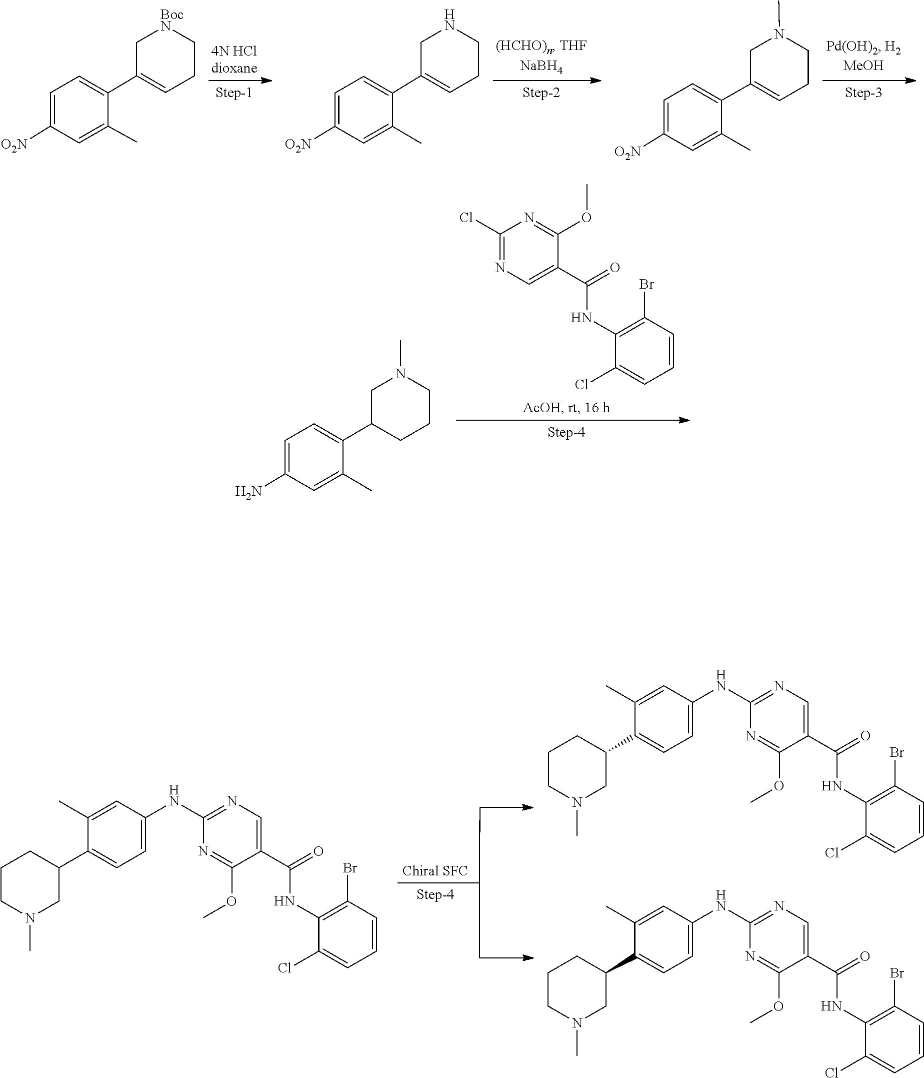

### 5-(2-methyl-4-nitrophenyl)-1,2,3,6-tetrahydropyridine

To a stirred solution of tert-butyl 5-(2-methyl-4-nitrophenyl)-3,6-dihydropyridine-1(2H)-carboxylate (800 mg, 2.51 mmol) in dioxane (10 mL), 4N HCl in dioxane (1.885 ml, 7.54 mmol) at 0° C. was added. The reaction mixture was stirred at room temperature for 2 h. Progress of the reaction was monitored by TLC. The reaction mixture was concentrated under reduced pressure to give 5-(2-methyl-4-nitrophenyl)-1,2,3,6-tetrahydropyridine (500 mg, 2.268 mmol, 90% yield). LCMS (ESI) m/z=219.0 (M+1).

### 1-methyl-5-(2-methyl-4-nitrophenyl)-1,2,3,6-tetrahydropyridine

To a stirred solution of 5-(2-methyl-4-nitrophenyl)-1,2,3,6-tetrahydropyridine (780 mg, 3.57 mmol) in TFE (15 mL) at 0° C., paraformaldehyde (536 mg, 17.87 mmol) was added and the resulting mixture was stirred at 75° C. for 2 h. The reaction mixture was cooled at 0° C., and sodium borohydride (311 mg, 8.22 mmol) was added to the reaction mixture. The reaction mixture was allowed to be stirred at 75° C. for 12 h. The reaction was monitored by TLC. The reaction mixture was diluted with water (100 mL) and extracted with 10% methanol-DCM (100 mL×3). Dried over anhydrous sodium sulfate, filtered, and concentrated under reduced pressure (bath temperature: 45° C.) to afford crude product. The crude product was purified by combi flash column chromatography (silica-gel, mesh size-100-200) using ethyl acetate/hexane (80-90%) as an eluent to obtain a 1-methyl-5-(2-methyl-4-nitrophenyl)-1,2,3,6-tetrahydropyridine (375 mg, 1.582 mmol, 44.3% yield). LCMS (ESI) im/z=233.0 (M+1).

### 3-methyl-4-(1-methylpiperidin-3-yl)aniline

To a degassed solution of 1-methyl-5-(2-methyl-4-nitrophenyl)-1,2,3,6-tetrahydropyridine (370 mg, 1.593 mmol) in MeOH (35.00 ml), was added 10% Pd—C(133 mg, 0.796 mmol) portion wise under nitrogen atmosphere. The reaction mixture was stirred for 16 h under the H₂-bladder (1 atm) at room temperature. Progress of the reaction was monitored by TLC. The reaction mixture was filtered through a small pad of celite which was washed with MeOH. The combined filtrate was concentrated to afford 3-methyl-4-(1-methylpiperidin-3-yl)aniline (315 mg, 1.511 mmol, 95% yield). LCMS (ESI) m/z=205.2 (M+1).

### N-(2-bromo-6-chlorophenyl)-4-methoxy-2-((3-methyl-4-(1-methylpiperidin-3-yl)phenyl)amino)pyrimidine-5-carboxamide

A mixture of 3-methyl-4-(1-methylpiperidin-3-yl)aniline (315 mg, 1.542 mmol) and N-(2-bromo-6-chlorophenyl)-2-chloro-4-methoxypyrimidine-5-carboxamide (581 mg, 1.542 mmol) in acetic acid (2 mL) was stirred at room temperature for 16 h. The progress of the reaction was monitored by TLC. The reaction mixture was concentrated under reduced pressure to give crude N-(2-bromo-6-chlorophenyl)-4-methoxy-2-((3-methyl-4-(1-methylpiperidin-3-yl)phenyl)amino)pyrimidine-5-carboxamide (208 mg, 0.366 mmol, 23.77% yield). LCMS (ESI) m/z=546.1 (M+2).

### Separation of (R)—N-(2-bromo-6-chlorophenyl)-4-methoxy-2-((3-methyl-4-(1-methylpiperidin-3-yl)phenyl)amino)pyrimidine-5-carboxamide and (S)—N-(2-bromo-6-chlorophenyl)-4-methoxy-2-((3-methyl-4-(1-methylpiperidin-3-yl)phenyl)amino)pyrimidine-5-carboxamide

Racemic N-(2-bromo-6-chlorophenyl)-4-methoxy-2-((3-methyl-4-(1-methylpiperidin-3-yl)phenyl)amino)pyrimidine-5-carboxamide was purified by Chiral SFC Purification. The collected fractions were concentrated under reduced pressure and then submitted for lyophilization. This afforded (R)—N-(2-bromo-6-chlorophenyl)-4-methoxy-2-((3-methyl-4-(1-methylpiperidin-3-yl)phenyl)amino)pyrimidine-5-carboxamide (38 mg) and (S)—N-(2-bromo-6-chlorophenyl)-4-methoxy-2-((3-methyl-4-(1-methylpiperidin-3-yl)phenyl)amino)pyrimidine-5-carboxamide (80.1 mg).

(R)—N-(2-bromo-6-chlorophenyl)-4-methoxy-2-((3-methyl-4-(1-methylpiperidin-3-yl)phenyl)amino)pyrimidine-5-carboxamide: LCMS (ESI) m/z=546.1 (M+2). ¹H-NMR (400 MHz, DMSO-d₆): δ 10.02 (s, 1H), 9.58 (s, 1H), 8.75 (s, 1H), 7.73 (dd, J=1.20, 8.00 Hz, 1H), 7.63-7.56 (m, 3H), 7.31 (t, J=8.40 Hz, 1H), 7.19 (d, J=8.40 Hz, 1H), 4.12 (s, 3H), 2.93-2.82 (m, 1H), 2.75-2.67 (m, 2H), 2.34 (s, 3H), 2.19 (s, 3H), 1.91-1.84 (m, 2H), 1.70-1.61 (m, 3H), 1.40-1.36 (m, 1H). (S)—N-(2-bromo-6-chlorophenyl)-4-methoxy-2-((3-methyl-4-(1-methylpiperidin-3-yl)phenyl)amino)pyrimidine-5-carboxamide. LCMS (ESI) m/z=546.2 (M+2). ¹H-NMR (400 MHz, DMSO-d₆): δ 10.04 (s, 1H), 9.58 (s, 1H), 8.75 (s, 1H), 7.73 (dd, J=0.80, 8.00 Hz, 1H), 7.63-7.58 (m, 1H), 7.31 (t, J=8.00 Hz, 1H), 7.19 (d, J=8.80 Hz, 1H), 4.12 (s, 3H), 2.97-2.89 (m, 3H), 2.34-2.28 (m, 6H), 2.17-2.08 (m, 2H), 1.76-1.66 (m, 3H), 1.48-1.44 (m, 1H).

### Example 82. Synthesis of (R)—N-(2-bromo-6-chlorophenyl)-4-methoxy-2-((3-methyl-4-(piperidin-3-yl)phenyl)amino)pyrimidine-5-carboxamide (Compound 198) and (S)—N-(2-bromo-6-chlorophenyl)-4-methoxy-2-((3-methyl-4-(piperidin-3-yl)phenyl)amino)pyrimidine-5-carboxamide (Compound 199)

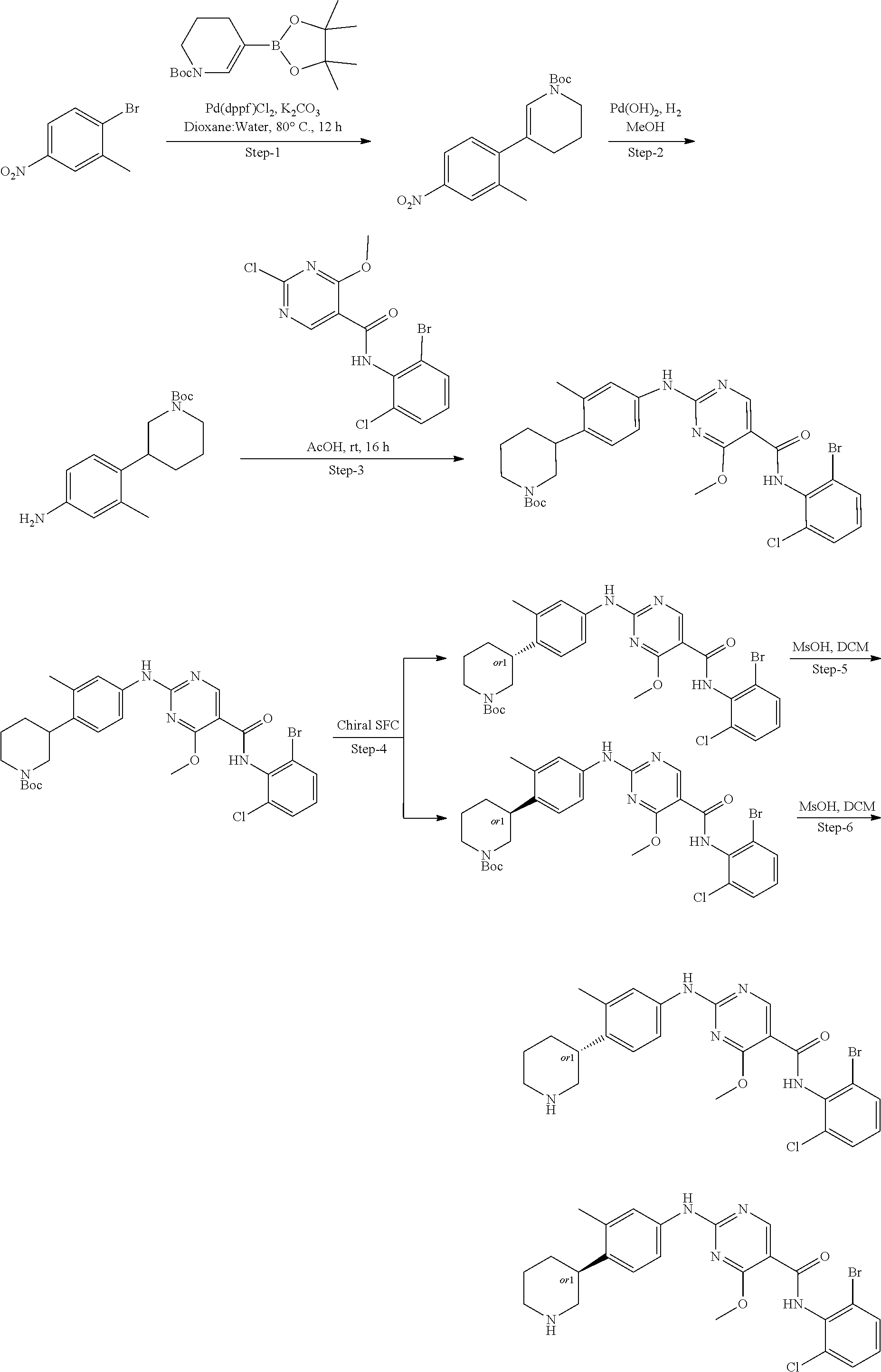

### tert-butyl 5-(2-methyl-4-nitrophenyl)-3,6-dihydropyridine-1(2H)-carboxylate

A degassed solution of 1-bromo-2-methyl-4-nitrobenzene (2 g, 9.26 mmol), tert-butyl 5-(4,4,5,5-tetramethyl-1,3,2-dioxaborolan-2-yl)-3,4-dihydropyridine-1(2H)-carboxylate (9.26 mmol), K₃PO₄ (4.91 g, 23.14 mmol) and Pd(dppf)Cl₂ (0.677 g, 0.926 mmol) in dioxane (45 ml) and water (5.000 ml) was stirred at 80° C. for 16 h. The progress of the reaction was monitored by TLC. The reaction mixture was diluted with water (100 mL) and extracted with 10% methanol-DCM (250 mL×3). The combined organic extract was washed with brine (150 mL), dried over anhydrous sodium sulfate, filtered, and concentrated under reduced pressure (bath temperature: 45° C.) to afford crude product. The crude product was purified by combi flash column chromatography (silica-gel, mesh size-100-200) using ethyl acetate/hexane (25-30%) as an eluent to obtain tert-butyl 5-(2-methyl-4-nitrophenyl)-3,6-dihydropyridine-1(2H)-carboxylate (2.8 g, 8.44 mmol, 91% yield). LCMS (ESI) m/z=319 [M+H], t_{R}=2.26 min, 96.22% (Method D).

### tert-butyl 5-(4-amino-2-methylphenyl)-3,6-dihydropyridine-1(2H)-carboxylate

To a degassed solution of tert-butyl 5-(2-methyl-4-nitrophenyl)-3,6-dihydropyridine-1(2H)-carboxylate (1.3 g, 4.08 mmol) in THE (15 ml) was added 10% Pd—C(0.136 g, 0.817 mmol) portion wise under nitrogen atmosphere. The reaction mixture was stirred for 16 h under the H₂-Bladder (1 atm) at room temperature. The progress of the reaction was monitored by TLC. The reaction mixture was filtered through a small pad of celite which was washed with MeOH. The combined organic extract was concentrated to afford tert-butyl 3-(4-amino-2-methylphenyl)piperidine-1-carboxylate (780 mg, 2.337 mmol, 57.2% yield). LCMS (ESI) m/z=191 [MH-100], t_{R}=1.89 min, 86.99% (Method D).

### tert-butyl 3-(4-((5-((2-bromo-6-chlorophenyl)carbamoyl)-4-methoxypyrimidin-2-yl)amino)-2-methylphenyl)piperidine-1-carboxylate

To a stirred solution of tert-butyl 3-(4-amino-2-methylphenyl)piperidine-1-carboxylate (400 mg, 1.377 mmol) in acetic acid (5 mL), N-(2-bromo-6-chlorophenyl)-2-chloro-4-methoxypyrimidine-5-carboxamide (519 mg, 1.377 mmol) was added. The reaction mixture was stirred at room temperature for 16 h. The progress of the reaction was monitored by TLC. The reaction mixture was concentrated under reduced pressure to give crude tert-butyl 3-(4-((5-((2-bromo-6-chlorophenyl)carbamoyl)-4-methoxypyrimidin-2-yl)amino)-2-methylphenyl)piperidine-1-carboxylate (310 mg, 0.462 mmol, 33.5% yield) which was submitted by SFC chiral purification. LCMS (ESI) m/z=632.46 [M+2], t_{R}=2.38 min, 94.08% (Method A).

### Separation of (R) tert-butyl 3-(4-((5-((2-bromo-6-chlorophenyl)carbamoyl)-4-methoxypyrimidin-2-yl)amino)-2-methylphenyl)piperidine-1-carboxylate and (S) tert-butyl 3-(4-((5-((2-bromo-6-chlorophenyl)carbamoyl)-4-methoxypyrimidin-2-yl)amino)-2-methylphenyl)piperidine-1-carboxylate

The crude tert-butyl 3-(4-((5-((2-bromo-6-chlorophenyl)carbamoyl)-4-methoxypyrimidin-2-yl)amino)-2-methylphenyl)piperidine-1-carboxylate was purified by chiral SFC to obtain both the isomers. After chiral SFC purification, isomers were concentrated under reduced pressure to give tert-butyl (R)-3-(4-((5-((2-bromo-6-chlorophenyl)carbamoyl)-4-methoxypyrimidin-2-yl)amino)-2-methylphenyl)piperidine-1-carboxylate (120 mg, 0.162 mmol, 32.9% yield) and tert-butyl (S)-3-(4-((5-((2-bromo-6-chlorophenyl)carbamoyl)-4-methoxypyrimidin-2-yl)amino)-2-methylphenyl)piperidine-1-carboxylate (140 mg, 0.213 mmol, 43.4% yield) as white solids.

(R) tert-butyl 3-(4-((5-((2-bromo-6-chlorophenyl)carbamoyl)-4-methoxypyrimidin-2-yl)amino)-2-methylphenyl)piperidine-1-carboxylate. LCMS (ESI) m/z=631 [M+H], tR=2.43 min, 81.03% (Method D).

(S) tert-butyl 3-(4-((5-((2-bromo-6-chlorophenyl)carbamoyl)-4-methoxypyrimidin-2-yl)amino)-2-methylphenyl)piperidine-1-carboxylate. LCMS (ESI) m/z=631 [M+H], tR=2.43 min, 96.78% (Method A).

### (R)-2-((1-(azepan-4-yl)-1H-pyrazol-4-yl)amino)-N-(2,6-dichlorophenyl)-4-methoxypyrimidine-5-carboxamide

To a stirred solution of tert-butyl (R)-3-(4-((5-((2-bromo-6-chlorophenyl)carbamoyl)-4-methoxypyrimidin-2-yl)amino)-2-methylphenyl)piperidine-1-carboxylate (140 mg, 0.222 mmol) in DCM (5 mL), methanesulfonic acid (171 mg, 1.775 mmol) was added at 0° C. The reaction mixture was stirred at room temperature for 2 h. The progress of the reaction was monitored by TLC. The reaction mixture was diluted with water (100 mL) and extracted with 10% methanol-DCM (100 mL×3), dried over anhydrous sodium sulfate, filtered and lyophilized to obtain (R)—N-(2-bromo-6-chlorophenyl)-4-methoxy-2-((3-methyl-4-(piperidin-3-yl)phenyl)amino)pyrimidine-5-carboxamide (90 mg, 0.166 mmol, 74.9% yield). LCMS (ESI) m/z=531 [M+H], tR=1.51 min, 97.23% (Method A). ¹H-NMR (400 MHz, DMSO-d₆): δ 10.05 (s, 1H), 9.55 (s, 1H), 8.75 (s, 1H), 8.35 (s, 1H), 7.73 (dd, J=1.20, 8.20 Hz, 1H), 7.63-7.58 (m, 3H), 7.31 (t, J=8.00 Hz, 1H), 7.20 (d, J=8.40 Hz, 1H), 4.12 (s, 3H), 3.14 (d, J=11.60 Hz, 1H), 3.06 (d, J=12.00 Hz, 1H), 2.96 (m, 1H), 2.69-2.67 (m, 2H), 2.33 (s, 3H), 1.81-1.79 (m, 2H), 1.68-1.63 (m, 2H).

### S)-2-((1-(azepan-4-yl)-1H-pyrazol-4-yl)amino)-N-(2,6-dichlorophenyl)-4-methoxypyrimidine-5-carboxamide

To a stirred solution t-butyl (S)-3-(4-((5-((2-bromo-6-chlorophenyl)carbamoyl)-4-methoxypyrimidin-2-yl)amino)-2-methylphenyl)piperidine-1-carboxylate (120 mg, 0.190 mmol) in DCM (5 mL), methanesulfonic acid (146 mg, 1.521 mmol) was added at 0° C. The reaction mixture was stirred at room temperature for 1 h. The progress of the reaction was monitored by TLC. The reaction mixture was diluted with water (100 mL) and extracted with 10% methanol-DCM (100 mL×3), dried over anhydrous sodium sulfate, filtered and lyophilized to obtain (S)—N-(2-bromo-6-chlorophenyl)-4-methoxy-2-((3-methyl-4-(piperidin-3-yl)phenyl)amino)pyrimidine-5-carboxamide (33 mg, 0.060 mmol, 31.7% yield). LCMS (ESI) m/z=532.4 [M+2], t_{R}=1.41 min, 98.86% (Method A). ¹H-NMR (400 MHz, DMSO-d₆): δ 10.01 (s, 1H), 9.58 (s, 1H), 8.75 (s, 1H), 7.73 (dd, J=6.80, Hz, 1H), 7.60-7.55 (m, 3H), 7.31 (t, J=8.40 Hz, 1H), 7.16 (d, J=8.40 Hz, 1H), 4.12 (s, 3H), 2.98-2.89 (m, 2H), 2.77-2.74 (m, 1H), 2.50 (m, 2H), 2.31 (s, 3H), 1.78-1.69 (m, 1H), 1.66-1.60 (m, 1H), 1.56-1.50 (m, 2H).

### Example 83. Synthesis of N-(2-bromo-6-chlorophenyl)-2-{1-[(S)-2-(dimethylamino)-1-methylethyl]-4-pyrazolylamino}-4-ethoxy-5-pyrimidinecarboxamide (Compound 202) and N-(2-bromo-6-chlorophenyl)-2-{1-[(R)-1-methyl-2-(methylamino)ethyl]-4-pyrazolylamino}-4-ethoxy-5-pyrimidinecarboxamide (Compound 203)

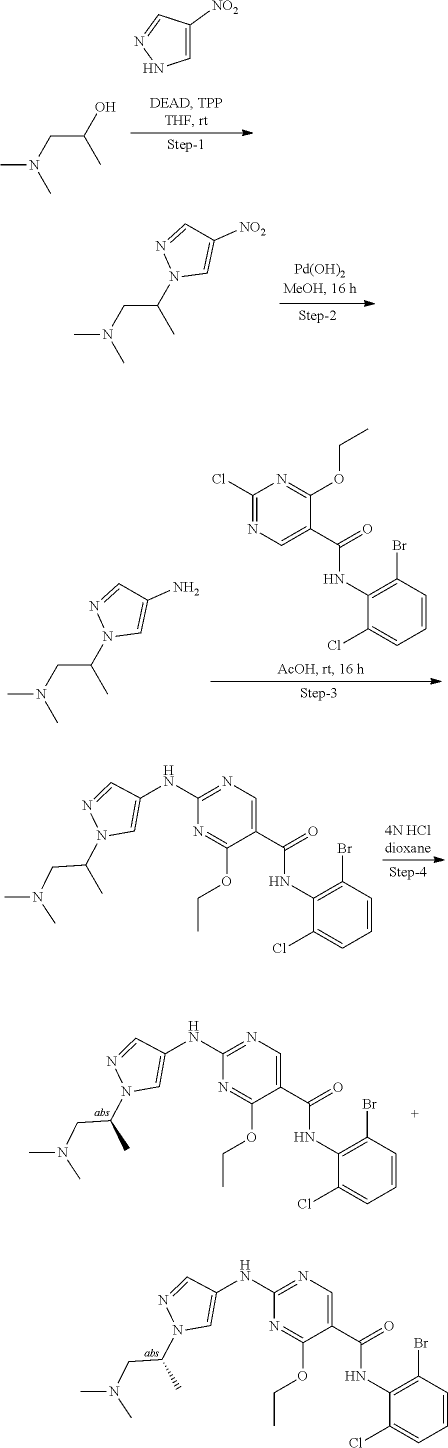

### N,N-dimethyl-2-(4-nitro-1H-pyrazol-1-yl)propan-1-amine

To a stirred solution of 4-nitro-1H-pyrazole (1 g, 8.84 mmol) in THF (10 ml) was added PPh₃ (3.48 g, 13.27 mmol), 1-(dimethylamino) propane-2-ol (0.912 g, 8.84 mmol) followed by the addition of DEAD (2.100 ml, 13.27 mmol) at 0° C. under inert atmosphere. The resulting mixture was then stirred at room temperature for 16 h. Progress of the reaction was monitored by TLC and LCMS. Water (25 mL) was added and the mixture extracted with ethyl acetate (2×250 mL), and the combined organic layers were dried over anhydrous Na₂SO₄, evaporated under reduced pressure to give the crude compound N,N-dimethyl-2-(4-nitro-1H-pyrazol-1-yl)propan-1-amine (350 mg, 1.642 mmol, 18.57% yield) as a gummy liquid which was taken as such for the next step.

### 1-(1-(dimethylamino)propan-2-yl)-1H-pyrazol-4-amine

To a stirred solution of N,N-dimethyl-2-(4-nitro-1H-pyrazol-1-yl) propane-1-amine (1.5 g, 7.57 mmol) in MeOH (10 ml) was added 10% Pd—C(0.805 g, 7.57 mmol) the resulting mixture was stirred under hydrogen bladder pressure at room temperature for 16 h. Progress of the reaction was monitored by TLC and LCMS. The reaction mixture was filtered a celite bed and concentrated under reduced pressure to give 1-(1-(dimethylamino)propan-2-yl)-1H-pyrazol-4-amine (1 g, 3.33 mmol, 44.0% yield) as a brown gummy solid.

### N-(2-bromo-6-chlorophenyl)-2-((1-(1-(dimethylamino)propan-2-yl)-1H-pyrazol-4-yl)amino)-4-ethoxypyrimidine-5-carboxamide

To a stirred solution of 1-(1-(dimethylamino) propane-2-yl)-1H-pyrazol-4-amine (300 mg, 1.783 mmol) in AcOH (7 ml) was added N-(2-bromo-6-chlorophenyl)-2-chloro-4-ethoxypyrimidine-5-carboxamide (500 mg, 1.279 mmol) at room temperature under inert atmosphere. The resulting mixture was stirred for 16 h. Progress of the reaction was monitored by TLC and LCMS. The solvent was evaporated under reduced pressure. To the residue was added 10% MeOH in DCM (400 mL) and this solution was washed with 10% NaHCO₃ (50 mL), and the organic layer was dried over anhydrous Na₂SO₄, concentrated under reduced pressure to give the crude compound, which was purified by prep. HPLC to get the pure compound N-(2-bromo-6-chlorophenyl)-2-((1-(1-(dimethylamino) propane-2-yl)-1H-pyrazol-4-yl)amino)-4-ethoxypyrimidine-5-carboxamide (330 mg, 0.631 mmol, 35.4% yield) as a white solid. LCMS (ESI) m/z=522.3 (M+H), t_{R}: 1.49, 95.08% (Method-D).

### Separation of (S)—N-(2-bromo-6-chlorophenyl)-2-((1-(1-(dimethylamino)propan-2-yl)-1H-pyrazol-4-yl)amino)-4-ethoxypyrimidine-5-carboxamide and (R)—N-(2-bromo-6-chlorophenyl)-2-((1-(1-(dimethylamino)propan-2-yl)-1H-pyrazol-4-yl)amino)-4-ethoxypyrimnidine-5-carboxamide

The racemic N-(2-bromo-6-chlorophenyl)-2-((1-(1-(dimethylamino) propane-2-yl)-1H-pyrazol-4-yl)amino)-4-ethoxypyrimidine-5-carboxamide was purified by chiral SFC (I-cellulose Z_0.5% IPAm in IPA_30_0_2.LCD, Flow: 4.0000 mL/min, Co-Solvent: 30.0%, Oven Temperature: 40, Column Position: 4: Column-4 BPR Pressure: 102.0 kgf/cm2) to obtain (S)—N-(2-bromo-6-chlorophenyl)-2-((1-(1-(dimethylamino)propan-2-yl)-1H-pyrazol-4-yl)amino)-4-ethoxypyrimidine-5-carboxamide (56 mg, 16.92% yield) as an off-white solid and (R)—N-(2-bromo-6-chlorophenyl)-2-((1-(1-(dimethylamino)propan-2-yl)-1H-pyrazol-4-yl)amino)-4-ethoxypyrimidine-5-carboxamide (54 mg, 16.33% yield) as an off-white solid.

(S)—N-(2-bromo-6-chlorophenyl)-2-((1-(1-(dimethylamino)propan-2-yl)-1H-pyrazol-4-yl)amino)-4-ethoxypyrimidine-5-carboxamide: LCMS (ESI) m/z=523.9 (M+H), t_{R}: 1.31, 99.43% (Method-A). ¹H-NMR (400 MHz, DMSO-d₆): δ 10.114 (s, 1H), 9.42 (s, 1H), 8.73 (s, 1H), 7.91 (s, 1H), 7.72 (d, J=1.20 Hz, 1H), 7.70-7.61 (m, 2H), 7.29 (t, J=8.00 Hz, 1H), 4.66 (q, J=6.80 Hz, 2H), 4.41 (q, J=6.80 Hz, 1H), 2.91-2.86 (m, 1H), 2.79-2.75 (m, 1H), 2.29 (s, 3H), 1.48 (t, J=7.20 Hz, 3H), 1.42 (d, J=6.80 Hz, 3H).

(R)—N-(2-bromo-6-chlorophenyl)-2-((1-(1-(dimethylamino)propan-2-yl)-1H-pyrazol-4-yl)amino)-4-ethoxypyrimidine-5-carboxamide: LCMS (ESI) m/z=523.7 (M+H), t_{R}: 1.333 min, 99.84% (Method-A). ¹H-NMR (400 MHz, DMSO-d₆): δ 10.11 (s, 1H), 9.44 (s, 1H), 8.72 (d, J=18.00 Hz, 1H), 7.94 (d, J=18.40 Hz, 1H), 7.72 (d, J=1.20 Hz, 1H), 7.60 (d, J=1.20 Hz, 1H), 7.56 (s, 1H), 7.33-7.29 (m, 1H), 4.66-4.58 (m, 2H), 4.17 (dd, J=7.20, 13.40 Hz, 1H), 3.98 (dd, J=6.40, 13.40 Hz, 1H), 3.00 (d, J=5.60 Hz, 1H), 2.19 (s, 6H), 1.49-1.46 (m, 3H), 0.87-0.85 (m, 3H)

### Example 84. Synthesis of 2-((4-(1,4-diazepan-1-yl)-2-methylphenyl)amino)-N-(2-bromo-6-chlorophenyl)-4-methoxypyrimidine-5-carboxamide (Compound 205)

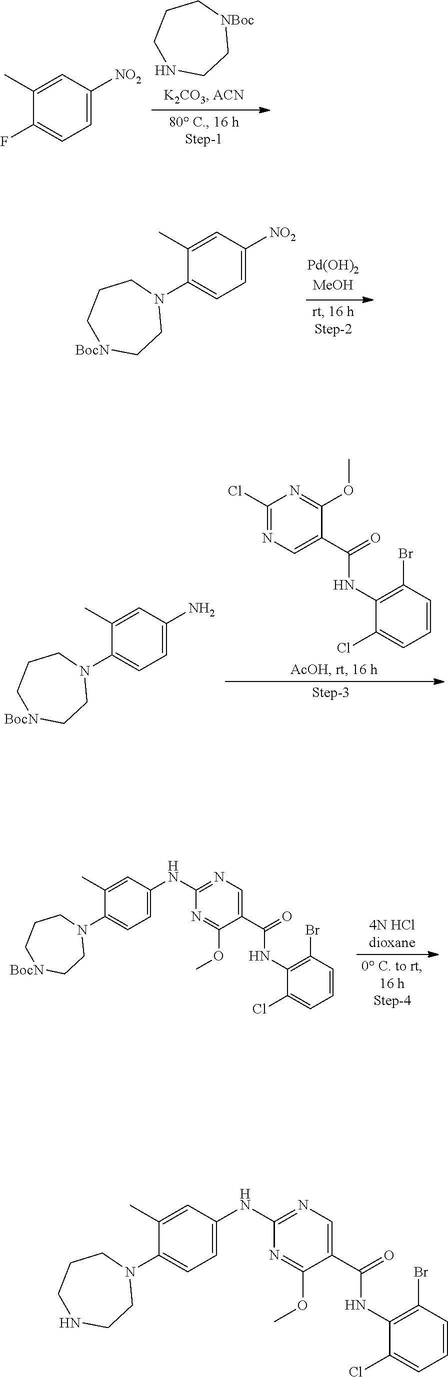

### tert-butyl 4-(2-methyl-4-nitrophenyl)-1,4-diazepane-1-carboxylate

To a stirred solution of 1-fluoro-2-methyl-4-nitrobenzene (1.5 g, 9.67 mmol) in acetonitrile (10 ml) were added K₂CO₃ (3.34 g, 24.17 mmol) followed by the addition of tert-butyl 1,4-diazepane-1-carboxylate (2.324 ml, 11.60 mmol). The resulting mixture was stirred for 16 h at 80° C. The progress of the reaction was monitored by TLC. The solvent was evaporated under reduced pressure and the residue diluted with water, and extracted with ethyl acetate to get crude compound which was purified by flash column chromatography (silica-gel, mesh size (230-400) using 10% ethyl acetate in hexane as eluent). Pure fractions were collected to obtain tert-butyl 4-(2-methyl-4-nitrophenyl)-1,4-diazepane-1-carboxylate (40 mg, 0.094 mmol, 0.974% yield) as a yellow solid compound. LCMS (ESI) m/z=336.0 (M+H), t_{R}: 1.91, 79.34% (Method-D)

### tert-butyl 4-(4-amino-2-methylphenyl)-1,4-diazepane-1-carboxylate

To a stirred solution of tert-butyl 4-(2-methyl-4-nitrophenyl)-1,4-diazepane-1-carboxylate (0.500 g, 1.491 mmol) in EtOH (20 ml) was added 10% Pd/C (0.248 g, 1.491 mmol). The resulting mixture was stirred under H₂ bladder pressure for 16 h at room temperature. Progress of the reaction was monitored by TLC and LCMS. The reaction mixture was filtered through celite bed, which was washed with excess MeOH (2×50 mL). The filtrate was then distilled to give crude compound tert-butyl 4-(4-amino-2-methylphenyl)-1,4-diazepane-1-carboxylate (450 mg, 1.223 mmol, 82% yield) as a brown gummy compound. This crude compound as such taken for the next step without any further purification. LCMS (ESI) m/z=306.3 (M+H), t_{R}:2.03, 83.92% (Method-D).

### tert-butyl 4-(4-((5-((2-bromo-6-chlorophenyl)carbamoyl)-4-methoxypyrimidin-2-yl)amino)-2-methylphenyl)-1,4-diazepane-1-carboxylate

To a stirred solution of tert-butyl 4-(4-amino-2-methylphenyl)-1,4-diazepane-1-carboxylate (0.400 g, 1.310 mmol) in AcOH (10 ml) was added N-(2-bromo-6-chlorophenyl)-2-chloro-4-methoxypyrimidine-5-carboxamide (0.444 g, 1.179 mmol). The reaction mixture was stirred for 16 h at room temperature. Progress of the reaction was monitored by TLC and LCMS. The solvent was evaporated and diluted with water and extracted with ethyl acetate (2×100 mL). The combined organic layers was washed with 10% NaHCO₃ and dried over anhydrous Na₂SO₄, and then concentrated under reduced pressure to give the crude compound, which was purified by flash column chromatography (silica-gel, mesh size (230-400) using 10-30% ethyl acetate in hexane as eluent) to get the pure compound tert-butyl 4-(4-((5-((2-bromo-6-chlorophenyl)carbamoyl)-4-methoxypyrimidin-2-yl)amino)-2-methylphenyl)-1,4-diazepane-1-carboxylate (350 mg, 0.515 mmol, 39.3% yield) as a yellow solid. LCMS (ESI) m/z=647.1 (M+2), t_{R}: 2.389, 94.58% (Method-D).

### 2-((4-(1,4-diazepan-1-yl)-2-methylphenyl)amino)-N-(2-bromo-6-chlorophenyl)-4-methoxypyrimidine-5-carboxamide

To a stirred solution of tert-butyl 4-(4-((5-((2-bromo-6-chlorophenyl)carbamoyl)-4-methoxypyrimidin-2-yl)amino)-3-methylphenyl)-1,4-diazepane-1-carboxylate (150 mg, 0.232 mmol) in 1,4-dioxane (10 ml) was added 4M HCl in dioxane (1.0 ml, 0.232 mmol) at 0° C. and the resulting mixture was allowed to stir at room temperature for 3 h. Progress of the reaction was monitored by TLC and LCMS. The solvent was evaporated the residue washed with n-hexane to get crude product which was purified by prep. HPLC to get the pure compound 2-((4-(1,4-diazepan-1-yl)-2-methylphenyl)amino)-N-(2-bromo-6-chlorophenyl)-4-methoxypyrimidine-5-carboxamide (35 mg, 0.063 mmol, 27.1% yield) as an off-white solid. LCMS (ESI) m/z=545.5 (M+H), t_{R}1.38, 93.44% (Method-A). ¹H-NMR (400 MHz, DMSO-d₆): δ 9.99 (s, 1H), 9.56 (s, 1H), 8.74 (s, 1H), 7.73 (dd, J=1.20, 8.20 Hz, 1H), 7.62-7.53 (m, 3H), 7.31 (t, J=8.00 Hz, 1H), 7.08-7.06 (m, 1H), 4.10 (s, 3H), 3.13-3.04 (m, 8H), 2.28 (s, 3H), 1.93-1.91 (m, 2H)

### Example 85. Synthesis of 2-((4-(1,4-diazepan-1-yl)-2-methylphenyl)amino)-N-(2-bromo-6-chlorophenyl)-4-methoxypyrimidine-5-carboxamide (Compound 207)

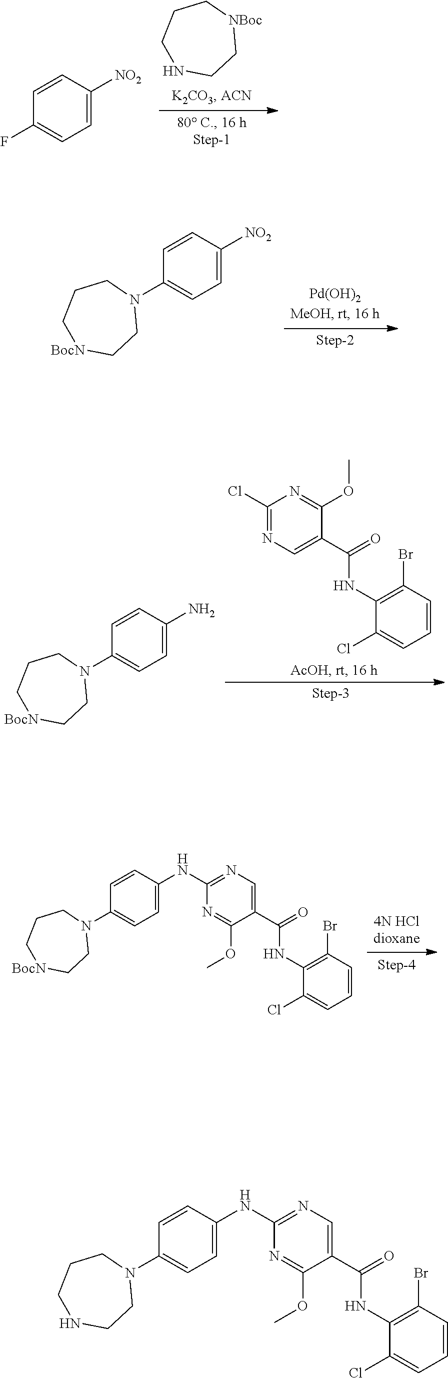

### tert-butyl 4-(4-nitrophenyl)-1,4-diazepane-f-carboxylate

To a stirred solution of 1-fluoro-4-nitrobenzene (1.0 g, 7.09 mmol) in acetonitrile (10 ml) was added K₂CO₃ (2.449 g, 17.72 mmol) and tert-butyl 1,4-diazepane-1-carboxylate (1.845 ml, 9.21 mmol) at room temperature under inert atmosphere. The mixture was stirred at 80° C. for 16 h. Progress of the reaction was monitored by TLC and LCMS. The mixture was concentrated under reduced pressure and the residue was diluted with water and extracted with ethyl acetate to give crude compound which was purified by flash column chromatography (silica-gel, mesh size (230-400) using 10-20% ethyl acetate in hexane as eluent) to give the pure compound tert-butyl 4-(4-nitrophenyl)-1,4-diazepane-1-carboxylate (1.5 g, 4.57 mmol, 64.5% yield) as a yellow solid. LCMS (ESI) m/z=322.2 (M+H), t_{R}:1.93, 98.03% (Method-D)

### tert-butyl 4-(4-aminophenyl)-1,4-diazepane-1-carboxylate

To a stirred solution of tert-butyl 4-(4-nitrophenyl)-1,4-diazepane-1-carboxylate (0.500 g, 1.556 mmol) in EtOH (10 ml) was added 10% Pd/C (0.200 g, 1.201 mmol) and the resulting mixture was allowed to stir under H₂ bladder pressure for 16 h. Progress of the reaction was monitored by TLC and LCMS. The reaction mixture was filtered through a celite bed which was washed with excess MeOH (2×50 mL). The filtrate was concentrated to give tert-butyl 4-(4-aminophenyl)-1,4-diazepane-1-carboxylate (400 mg, 1.373 mmol, 88% yield) as a brown gummy solid. LCMS (ESI) m/z=292.0 (M+H), t_{R}:1.248, 96.46% (Method-A).

### tert-butyl 4-(4-((5-((2-bromo-6-chlorophenyl)carbamoyl)-4-methoxypyrimidin-2-yl)amino)phenyl)-1,4-diazepane-1-carboxylate

To a stirred solution of tert-butyl 4-(4-aminophenyl)-1,4-diazepane-1-carboxylate (400 mg, 1.373 mmol) in acetic acid (5.0 ml) was added N-(2-bromo-6-chlorophenyl)-2-chloro-4-methoxypyrimidine-5-carboxamide (466 mg, 1.235 mmol) and the resulting mixture was allowed to stir at room temperature for 16 h. Progress of the reaction was monitored by TLC and LCMS. The solvent was evaporated under reduced pressure and the residue then diluted with water and extracted with ethyl acetate (2×100 mL). The organic layer was washed with 10% NaHCO₃ (100 mL), dried over anhydrous Na₂SO₄ and concentrated under reduced pressure to give the crude compound which was purified by flash column chromatography (silica-gel, mesh size (230-400) using 80-100% ethyl acetate in hexane as eluent) to give the pure compound tert-butyl 4-(4-((5-((2-bromo-6-chlorophenyl)carbamoyl)-4-methoxypyrimidin-2-yl)amino)phenyl)-1,4-diazepane-1-carboxylate (400 mg, 0.462 mmol, 33.7% yield) as a yellow solid compound. LCMS (ESI) m/z=633.1 (M+2), t_{R}:2.203, 73.90% (Method-D).

### 2-((4-(1,4-diazepan-1-yl)phenyl)amino)-N-(2-bromo-6-chlorophenyl)-4-methoxypyrimidine-5-carboxamide

To a stirred solution of tert-butyl 4-(4-((5-((2-bromo-6-chlorophenyl)carbamoyl)-4-methoxypyrimidin-2-yl)amino)phenyl)-1,4-diazepane-1-carboxylate (100 mg, 0.158 mmol) in DCM (5 ml) was added methanesulphonic acid (1.0 ml, 0.158 mmol) at 0° C. under inert atmosphere. The mixture was allowed to stir at room temperature for 3 h. Progress of the reaction was monitored by TLC and LCMS. The reaction mixture was concentrated under reduced pressure and the residue diluted with water and extracted with 20% MeOH in DCM (2×100 mL). The organic layer was washed with 10% aq. NaHCO₃, dried over anhydrous Na₂SO₄ and concentrated under reduced pressure to give the crude compound which was purified by prep. HPLC to give 2-((4-(1,4-diazepan-1-yl)phenyl)amino)-N-(2-bromo-6-chlorophenyl)-4-methoxypyrimidine-5-carboxamide (19.98 mg, 0.462 mmol, 20.7% yield) as a pale-yellow solid. LCMS (ESI) m/z=531.3 (M+H), t_{R}: 1.29, 96.93% (Method-A). ¹H-NMR (400 MHz, DMSO-d₆): δ 9.87 (s, 1H), 9.50 (s, 1H), 8.70 (s, 1H), 7.72 (dd, J=1.20, 8.00 Hz, 1H), 7.62-7.60 (m, 3H), 7.30 (t, J=8.40 Hz, 1H), 6.70 (d, J=8.80 Hz, 2H), 4.08 (s, 3H), 3.53-3.52 (m, 4H), 2.95-2.92 (m, 2H), 2.75-2.68 (m, 2H), 1.86-1.83 (m, 2H)

### Example 86. Synthesis of N-(2-bromo-6-chlorophenyl)-4-methoxy-2-((3-methoxy-4-(1-methylpiperidin-4-yl)phenyl)amino)pyrimidine-5-carboxamide (Compound 216)

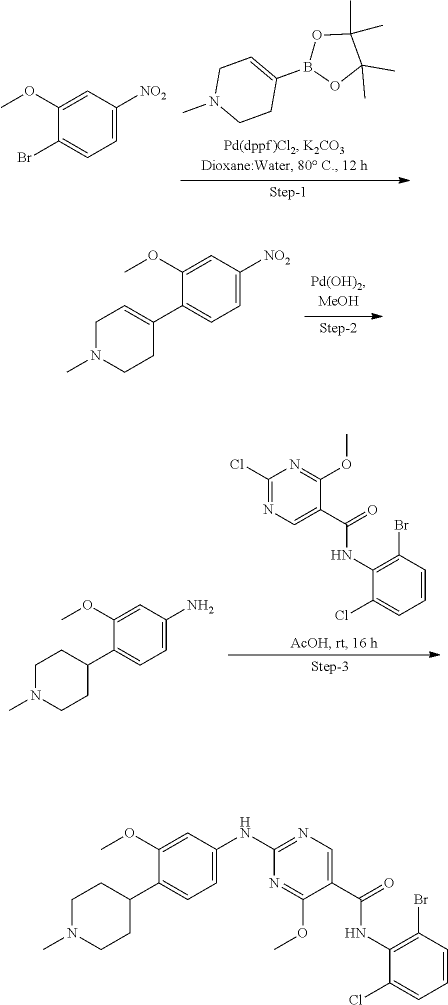

### 4-(2-methoxy-4-nitrophenyl)-1-methyl-1,2,3,66-tetrahydropyridine

A degassed solution of 1-bromo-2-methoxy-4-nitrobenzene (1 g, 4.31 mmol), 1-methyl-4-(4,4,5,5-tetramethyl-1,3,2-dioxaborolan-2-yl)-1,2,3,6-tetrahydropyridine (1.250 g, 5.60 mmol), Pd(dppf)Cl₂ (0.315 g, 0.431 mmol) and K₂CO₃ (1.489 g, 10.77 mmol) in dioxane (8 ml) and water (2.00 ml) was reacted under MW irradiation at 100° C. for 2 h. The progress of the reaction was monitored by UPLC. The reaction mixture was diluted with water and extracted with 10% MeOH:DCM. The combined organic layers were concentrated under reduced pressure. The resulting crude material was purified by flash chromatography (SiO₂/100-200 mesh; 6-12% MeOH/DCM) to afford 4-(2-methoxy-4-nitrophenyl)-1-methyl-1,2,3,6-tetrahydropyridine (0.85 g, 2.92 mmol, 67.8% yield) as a colorless gummy solid. LCMS (ESI) m/z=249.4 (M+1).

### 3-methoxy-4-(1-methylpiperidin-4-yl)aniline

To a degassed solution of 4-(2-methoxy-4-nitrophenyl)-1-methyl-1,2,3,6-tetrahydropyridine (600 mg, 2.417 mmol) in MeOH (8 ml) and THE (2 ml) was added Pd(OH)₂ on carbon (50.9 mg, 0.362 mmol) under inert atmosphere. The resulting mixture was stirred for 12 h under a hydrogen bladder atmosphere. The progress of the reaction was monitored by UPLC. The reaction mixture was filtered through a small celite pad which was washed with methanol. The filtrate was concentrated under reduced pressure to get crude 3-methoxy-4-(1-methylpiperidin-4-yl)aniline (780 mg, 2.160 mmol, 89% yield) as brownish gummy solid. LCMS (ESI) m/z=220.8 (M+1).

### N-(2-bromo-6-chlorophenyl)-4-methoxy-2-((3-methoxy-4-(1-methylpiperidin-4-yl)phenyl)amino)pyrimidine-5-carboxamide

A mixture of N-(2-bromo-6-chlorophenyl)-2-chloro-4-methoxypyrimidine-5-carboxamide (0.411 g, 1.089 mmol) and N-(2-bromo-6-chlorophenyl)-2-chloro-4-methoxypyrimidine-5-carboxamide (0.411 g, 1.089 mmol) in acetic acid (5 ml) was stirred at room temperature for 12 h. The progress of the reaction is monitored by LCMS. The solvent was evaporated and the residue washed with a small amount of aqueous NaHCO₃ solution and re-extracted with 10% MeOH-DCM solution. The combined organic layer was evaporated under reduced pressure and the residue purified by prep. HPLC. Pure fractions were collected and lyophilized to get N-(2-bromo-6-chlorophenyl)-4-methoxy-2-((3-methoxy-4-(1-methylpiperidin-4-yl)phenyl)amino)pyrimidine-5-carboxamide (55 mg, 0.096 mmol, 10.59% yield) as a brown solid. LCMS (ESI)/z=562.1 (M+1), t_{R}=1.55 min, 98.71% (Method-D). ¹H-NMR (400 MHz, DMSO-d₆): δ 10.10 (s, 1H), 9.60 (s, 1H), 8.77 (s, 1H), 7.73 (dd, J=1.20, 8.20 Hz, 1H), 7.61 (d, J=6.80 Hz, 2H), 7.33-7.24 (m, 2H), 7.11 (d, J=8.40 Hz, 1H), 4.14 (s, 3H), 3.80 (s, 3H), 2.86 (d, J=11.20 Hz, 2H), 2.78-2.75 (m, 1H), 2.19 (s, 3H), 1.95-1.93 (m, 2H), 1.66-1.61 (m, 4H).

### Example 87. Synthesis of N-(2-bromo-6-chlorophenyl)-2-((3-ethoxy-4-(1-methylpiperidin-4-yl)phenyl)amino)-4-methoxypyrimidine-5-carboxamide (Compound 217)

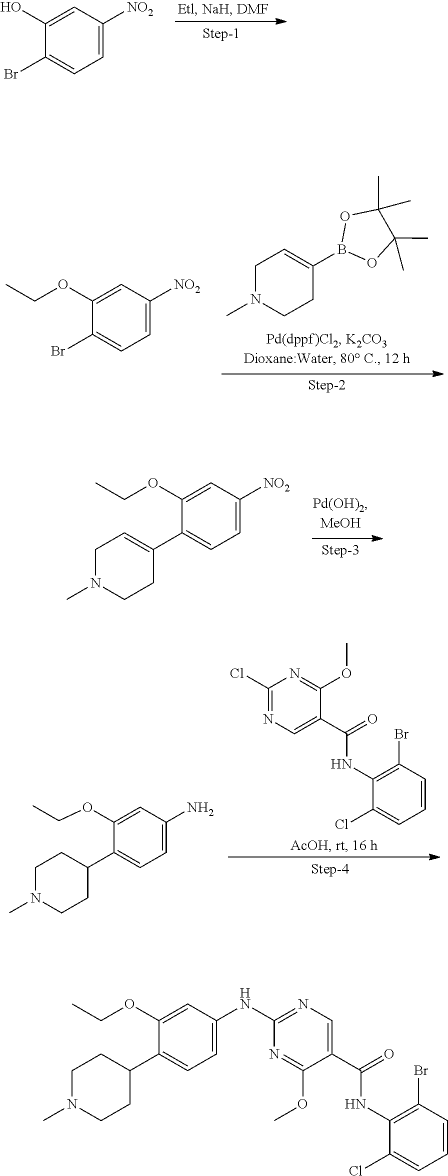

### 1-bromo-2-ethoxy-4-nitrobenzene

To a degassed solution of 2-bromo-5-nitrophenol (2 g, 9.17 mmol) in DMF (25 ml) was added sodium hydride (0.264 g, 11.01 mmol) portion-wise at 0° C. and the resulting mixture was stirred for 5 min, whereupon iodoethane (0.890 ml, 11.01 mmol) was added dropwise at 0° C. The reaction mixture was stirred at 25° C. for 4 h. The progress of the reaction was monitored by TLC. The reaction mixture was diluted with water (100 mL) and extracted with 10% methanol-DCM (250 mL×3). The combined organic extract was washed with sodium bicarbonate (150 mL), dried over anhydrous sodium sulfate, filtered, and concentrated under reduced pressure (bath temperature: 45° C.). The resulting crude material was purified by flash chromatography (SiO₂/100-200 mesh; 10-15% EtOAc/hexane) to afford 1-bromo-2-ethoxy-4-nitrobenzene (2 g, 8.13 mmol, 89% yield) as a colorless gummy solid.

### 4-(2-ethoxy-4-nitrophenyl)-1-methyl-1,2,3,6-tetrahydropyridine

To a degassed solution of 1-bromo-2-ethoxy-4-nitrobenzene (1 g, 4.06 mmol) in dioxane (45 ml) and water (5.000 ml), 1-methyl-4-(4,4,5,5-tetramethyl-1,3,2-dioxaborolan-2-yl)-1,2,3,6-tetrahydropyridine (0.907 g, 4.06 mmol), K₃PO₄ (2.157 g, 10.16 mmol) and Pd(dppf)cl₂ (0.297 g, 0.406 mmol) were added and the resulting mixture was stirred at 80° C. for 12 h. The progress of the reaction was monitored by TLC. The reaction mixture was diluted with water (100 mL) and extracted with 10% methanol-DCM (250 mL×3). The combined organic extract was washed with brine (150 mL), dried over anhydrous sodium sulfate, filtered, and concentrated under reduced pressure (bath temperature: 45° C.) to afford crude product. The crude product was purified by combi flash column chromatography (silica-gel, mesh size-100-200) using ethyl acetate/hexane (25-30%) as an eluent to obtain a 4-(2-ethoxy-4-nitrophenyl)-1-methyl-1,2,3,6-tetrahydropyridine (800 mg, 1.860 mmol, 45.8% yield). LCMS (ESI) m/z=263.70 (M+1).

### 3-ethoxy-4-(1-methylpiperidin-4-yl)aniline

To a degassed solution of 4-(2-ethoxy-4-nitrophenyl)-1-methyl-1,2,3,6-tetrahydropyridine (800 mg, 3.05 mmol) in MeOH (20.0 ml), was added, palladium hydroxide on carbon (171 mg, 1.220 mmol) portion wise. The reaction mixture was stirred under hydrogen atmosphere for 12 h at room temperature. The progress of the reaction was monitored by TLC. The reaction mixture was filtered through a small pad of celite which was washed with MeOH. The combined organic filtrate was concentrated to afford 3-ethoxy-4-(1-methylpiperidin-4-yl)aniline (550 mg, 2.018 mmol, 66.2% yield). LCMS (ESI) m/z=235.1 (M+1).

### N-(2-bromo-6-chlorophenyl)-2-((3-ethoxy-4-(1-methylpiperidin-4-yl)phenyl)amino)-4-methoxypyrimidine-5-carboxamide

To a stirred solution of 3-ethoxy-4-(1-methylpiperidin-4-yl)aniline (149 mg, 0.637 mmol) in DCM (5 mL) was added N-(2-bromo-6-chlorophenyl)-2-chloro-4-methoxypyrimidine-5-carboxamide (200 mg, 0.530 mmol) followed by acetic acid (31.9 mg, 0.530 mmol) at room temperature. The reaction mixture was stirred at room temperature for 12 h. The progress of the reaction was monitored by TLC. Acetic acid was evaporated under reduced pressure and the resulting crude compound was purified by prep. HPLC. The fraction was lyophilized to get N-(2-bromo-6-chlorophenyl)-2-((3-ethoxy-4-(1-methylpiperidin-4-yl)phenyl)amino)-4-methoxypyrimidine-5-carboxamide (183 mg, 0.312 mmol, 58.8% yield). LCMS (ESI) m/z=575.9 (M+1). ¹H-NMR (400 MHz, DMSO-d₆): δ 10.07 (s, 1H), 9.59 (s, 1H), 8.76 (s, 1H), 7.73-7.63 (m, 1H), 7.63-7.61 (m, 1H), 7.60 (d, J=1.20 Hz, 1H), 7.56-7.26 (m, 2H), 7.10 (d, J=8.40 Hz, 1H), 4.13 (s, 3H), 4.02 (q, J=12.00 Hz, 2H), 2.92-2.80 (m, 2H), 2.78-2.77 (m, 1H), 2.23 (s, 3H), 2.04-2.00 (m, 2H), 1.67-1.63 (m, 4H), 1.36 (t, J=6.80 Hz, 3H).

### Example 88. Synthesis of N-(2-bromo-6-chlorophenyl)-4-methoxy-2-((4-(1-methylpiperidin-4-yl)-3-propoxyphenyl)amino)pyrimidine-5-carboxamide (Compound 218)

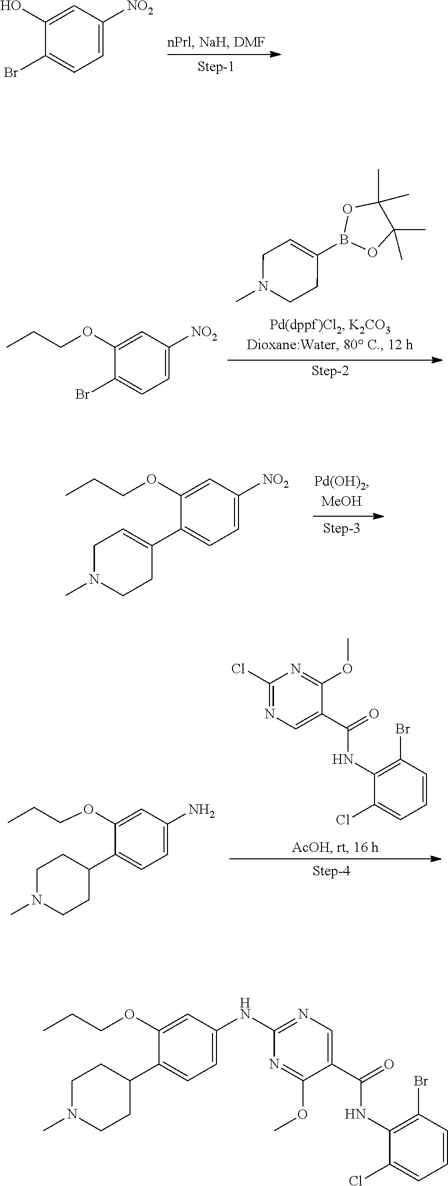

### 1-bromo-4-nitro-2-propoxybenzene

To a stirred solution of 2-bromo-5-nitrophenol (2 g, 9.17 mmol) in DMF (25 ml) at 0° C., sodium hydride (0.264 g, 11.01 mmol) was added. After 20 min iodopropane (0.985 ml, 11.01 mmol) was added at 0° C. The reaction mixture was allowed to be stirred at room temperature for 2 h. The progress of the reaction was monitored by TLC. The reaction mixture was quenched with ice, then diluted with ice-cold water (1000 mL), and extracted with ethyl acetate (500×3). The combined organic extract was washed with saturated aqueous sodium bicarbonate solution (250 mL). The organic layer was dried over anhydrous sodium sulfate, filtered, and concentrated under reduced pressure. The resulting crude material was purified by flash chromatography (SiO₂/100-200 mesh; 6-12% EtOAc/hexanes) to get a 1-bromo-4-nitro-2-propoxybenzene (2.3 g, 6.01 mmol, 65.5% yield) as a pale brown sticky solid.

### 1-methyl-4-(4-nitro-2-propoxyphenyl)-1,2,3,6-tetrahydropyridine

To a stirred solution of 1-bromo-4-nitro-2-propoxybenzene (1.6 g, 6.15 mmol) in 1,4-dioxane (45 ml) and water (5.000 ml) K₃PO₄ (3.26 g, 15.38 mmol) and 1-methyl-4-(4,4,5,5-tetramethyl-1,3,2-dioxaborolan-2-yl)-1,2,3,6-tetrahydropyridine (1.373 g, 6.15 mmol) were added. The mixture was then bubbled with nitrogen for 15 min before Pd(dppf)Cl₂ DCM complex (0.502 g, 0.615 mmol) was added. The reaction mixture was stirred at 80° C. for 16 h. The progress of the reaction was monitored by TLC. The reaction mixture was diluted with water (500 mL) and extracted with 10% methanol-DCM (100 mL×3). The combined organic extract was washed with brine (250 mL), dried over anhydrous sodium sulfate, filtered, and concentrated under reduced pressure (bath temperature: 45° C.) to afford 1-methyl-4-(4-nitro-2-propoxyphenyl)-1,2,3,6-tetrahydropyridine (820 mg, 2.97 mmol, 48.2% yield). LCMS (ESI) m/z=277.4 (M+1).

### 4-(1-methylpiperidin-4-yl)-3-propoxyaniline

To a stirred solution of 1-methyl-4-(4-nitro-2-propoxyphenyl)-1,2,3,6-tetrahydropyridine (800 mg, 2.90 mmol) in methanol (30 ml), Pd(OH)₂ (203 mg, 0.290 mmol) was added under inert atmosphere at room temperature portion-wise. The reaction mixture was stirred at room temperature for 16 h under a hydrogen atmosphere. The progress of the reaction was monitored by TLC. The reaction mixture was filtered through a celite bed, and the bed was washed with methanol (500 mL). The filtrate was concentrated under reduced pressure (bath temperature: 45° C.) to afford crude 4-(1-methylpiperidin-4-yl)-3-propoxyaniline. The crude material is taken as such for the next step.

### N-(2-bromo-6-chlorophenyl)-4-methoxy-2-((4-(1-methylpiperidin-4-yl)-3-propoxyphenyl)amino)pyrimidine-5-carboxamide

To a stirred solution of 4-(1-methylpiperidin-4-yl)-3-propoxyaniline (350 mg, 1.409 mmol) in acetic acid (6 ml), was added N-(2-bromo-6-chlorophenyl)-2-chloro-4-methoxypyrimidine-5-carboxamide (531 mg, 1.409 mmol) at room temperature. The reaction mixture was then stirred for 12 h room temperature. The progress of reaction was monitored by TLC. The reaction mixture was concentrated under reduced pressure to remove acetic acid. The reaction mixture was diluted with ice-cold water (50 mL) and extracted with solvent 10% Methanol:DCM (30 mL×3). The combined organic extract was washed with saturated aqueous sodium bicarbonate solution (100 mL). The organic layer was washed with brine (50 mL), dried over anhydrous sodium sulfate, filtered, and concentrated under reduced pressure (bath temperature: 45° C.) to afford crude compound. The crude product was further purified by reverse phase column chromatography; pure fractions were collected and lyophilized to afford pure N-(2-bromo-6-chlorophenyl)-4-methoxy-2-((4-(1-methylpiperidin-4-yl)-3-propoxyphenyl)amino)pyrimidine-5-carboxamide (90.4 mg, 0.148 mmol, 10.50% yield) as an off-white solid. LCMS (ESI) im/z=589.4 (M+1). ¹H-NMR (400 MHz, DMSO-d₆): δ 9.78 (s, 1H), 9.42 (s, 1H), 8.76 (s, 1H), 7.71 (dd, J=1.20, 8.00 Hz, 1H), 7.60-7.58 (m, 1H), 7.53 (d, J=2.00 Hz, 1H), 7.32-7.27 (m, 2H), 7.10 (d, J=8.40 Hz, 1H), 4.16 (s, 1H), 4.05 (t, J=6.40 Hz, 2H), 3.95-3.54 (m, 1H), 3.20-3.09 (m, 1H), 2.90-2.68 (m, 2H), 2.21 (s, 3H), 2.01-1.97 (m, 2H), 1.79-1.66 (m, 5H), 1.06 (t, J=3.60 Hz, 3H).

### Example 89. Synthesis of N-(2-bromo-6-chlorophenyl)-4-methoxy-2-((3-methyl-4-((1-methylpiperidin-4-yl)oxy)phenyl)amino)pyrimidine-5-carboxamide (Compound 220)

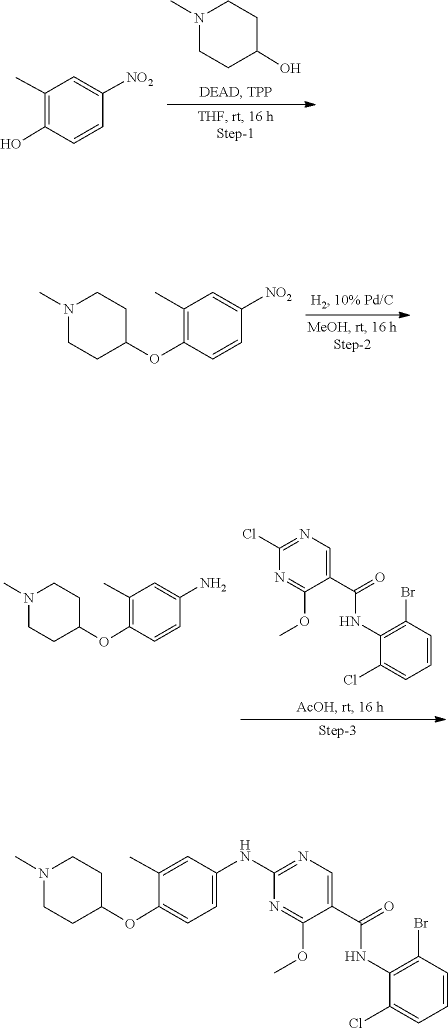

### tert-butyl 4-(2-methyl-4-nitrophenoxy)piperidine-1-carboxylate

To a stirred solution of 2-methyl-4-nitrophenol (2 g, 13.06 mmol) in THE (20 ml) were added triphenylphosphine (5.14 g, 19.59 mmol) and DEAD (2.274 g, 13.06 mmol) at 0° C. followed by the addition of tert-butyl 4-hydroxypiperidine-1-carboxylate (2.63 g, 13.06 mmol) at room temperature under inert atmosphere. The mixture was then stirred at room temperature for 16 h. Progress of the reaction was monitored by TLC and LCMS. The reaction mixture was concentrated under reduced pressure, diluted with water (100 mL) and extracted with ethyl acetate (3×250 mL), combined organic extracts and washed with brine (100 mL), dried over anhydrous Na₂SO₄, filtered and concentrated under reduced pressure to afford tert-butyl 4-(2-methyl-4-nitrophenoxy)piperidine-1-carboxylate (500 mg, 0.684 mmol, 5.24% yield) as a brown gummy solid.

### 3-methyl-4-((1-methylpiperidin-4-yl)oxy)aniline

To the stirred solution of 1-methyl-4-(2-methyl-4-nitrophenoxy)piperidine (400 mg, 1.598 mmol) in EtOH (10 ml) was added 10% Pd/C (224 mg, 1.598 mmol) at 25° C. under inert atmosphere, and the resulting mixture was allowed to stir under H₂ bladder pressure at room temperature for 16 h. Progress of the reaction was monitored by TLC and LCMS. The reaction mixture was filtered through celite, and the filtrate was concentrated under reduced pressure to give 3-methyl-4-((1-methylpiperidin-4-yl)oxy)aniline (295 mg, 1.339 mmol, 84% yield) as a brown gummy solid. LCMS (ESI) m/z=221.2 (M+H), t_{R}: 0.246, 90.26% (Method-A)

### N-(2-bromo-6-chlorophenyl)-4-methoxy-2-((3-methyl-4-((1-methylpiperidin-4-yl)oxy)phenyl)amino)pyrimidine-5-carboxamide

To a stirred solution of 3-methyl-4-((1-methylpiperidin-4-yl)oxy)aniline (150 mg, 0.681 mmol) in acetic acid (10 ml) was added N-(2-bromo-6-chlorophenyl)-2-chloro-4-methoxypyrimidine-5-carboxamide (257 mg, 0.681 mmol) at room temperature under inert atmosphere. The reaction mixture was then allowed to stir at room temperature for 16 h. Progress of the reaction was monitored by TLC and LCMS. The reaction mixture was concentrated under reduced pressure and water (100 mL) was added and the mixture then extracted with 10% MeOH in DCM (2×100 mL). The combined organic layers was washed with 10% aq. sodium bicarbonate solution, dried over anhydrous Na₂SO₄ and concentrated under reduced pressure to give crude compound which was purified by prep. HPLC to give N-(2-bromo-6-chlorophenyl)-4-methoxy-2-((3-methyl-4-((1-methylpiperidin-4-yl)oxy)phenyl)amino)pyrimidine-5-carboxamide (82 mg, 0.146 mmol, 24.41% yield). LCMS (ESI) im/z=560.0 (M+H), t_{R}1.44, 99.75% (Method-A). ¹H-NMR (400 MHz, DMSO-d₆): δ 9.93 (s, 1H), 9.54 (s, 1H), 8.73 (s, 1H), 7.73 (d, J=1.20 Hz, 1H), 7.71 (d, J=1.20 Hz, 1H), 7.61 (q, J=0.80 Hz, 2H), 7.31 (t, J=8.00 Hz, 1H), 6.95 (d, J=8.80 Hz, 1H), 4.31 (s, 1H), 4.09 (s, 3H), 2.68-2.67 (m, 2H), 2.22-2.17 (m, 9H), 1.91-1.76 (m, 4H), 1.68-1.66 (m, 2H)

### Example 90. Synthesis of N-(2,6-dichloro-3-hydroxyphenyl)-4-methoxy-2-((1-methyl-1H-pyrazol-4-yl)amino)pyrimidine-5-carboxamide (Compound 223)

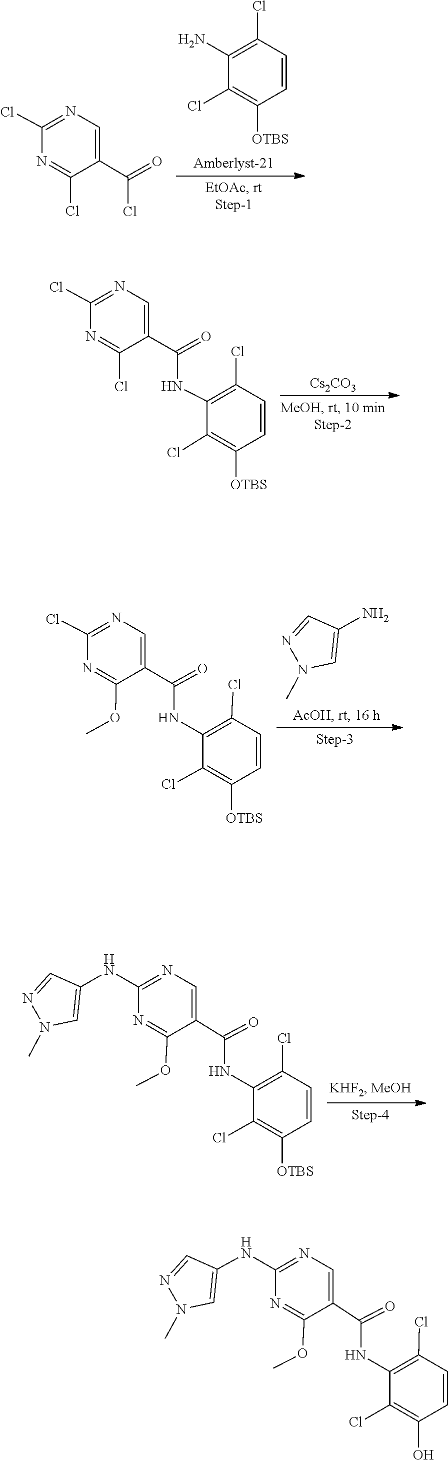

### N-(3-((tert-butyldimethylsilyl)oxy)-2,6-dichlorophenyl)-2,4-dichloropyrimidine-5-carboxamide

To a stirred solution of 2,4-dichloropyrimidine-5-carbonyl chloride (0.5 ml, 1.369 mmol) in ethyl acetate (150 ml) was added Amberlyst A 21 free base (150 mg, 1.369 mmol) followed by 3-((tert-butyldimethylsilyl)oxy)-2,6-dichloroaniline (400 mg, 1.369 mmol) at 0° C. under inert atmosphere. The resulting reaction mixture was stirred at room temperature for 16 h. Progress of the reaction was monitored by TLC and LCMS. The reaction mixture was filtered through celite and concentrated under reduced pressure. The residue was purified by (SiO₂/230-400 mesh; 5-20% MeOH-DCM) to give N-(3-((tert-butyldimethylsilyl)oxy)-2,6-dichlorophenyl)-2,4-dichloropyrimidine-5-carboxamide (400 mg, 0.856 mmol, 62.6% yield) as off white solid. LCMS (ESI) m/z=465.9 (M+H), t_{R}: 2.52, 74.27% (Method-B).

### N-(3-((tert-butyldimethylsilyl)oxy)-2,6-dichlorophenyl)-2-chloro-4-methoxypyrimidine-5-carboxamide

To a stirred solution of N-(3-((tert-butyldimethylsilyl)oxy)-2,6-dichlorophenyl)-2,4-dichloropyrimidine-5-carboxamide (400 mg, 0.856 mmol) in MeOH (10 ml) was added cesium carbonate (223 mg, 0.685 mmol) at 0° C. The resulting reaction mixture was stirred at room temperature for 3 h. Progress of the reaction was monitored by TLC and LCMS. Water was added and the resulting mixture extracted with DCM. The organic phase was dried over anhydrous Na₂SO₄, concentrated under reduced pressure to give N-(3-((tert-butyldimethylsilyl)oxy)-2,6-dichlorophenyl)-2-chloro-4-methoxypyrimidine-5-carboxamide (350 mg, 0.575 mmol, 67.1% yield) as yellow semi-solid. This compound, as such, is taken for the next step without any further purification. LCMS (ESI) m/z=462.9 (M+H), t_{R}: 2.35, 76.06% (Method-A)

### N-(3-((tert-butyldimethylsilyl)oxy)-2,6-dichlorophenyl)-4-methoxy-2-((1-methyl-1H-pyrazol-4-yl)amino)pyrimidine-5-carboxamide

To a stirred solution of N-(3-((tert-butyldimethylsilyl)oxy)-2,6-dichlorophenyl)-2-chloro-4-methoxypyrimidine-5-carboxamide (200 mg, 0.432 mmol) in AcOH (10 ml) was added 1-methyl-1H-pyrazol-4-amine (84 mg, 0.864 mmol) at room temperature under inert atmosphere. The resulting reaction mixture was stirred at 25° C. for 16 h. Progress of the reaction was monitored by TLC and LCMS. The reaction mixture was diluted with water and extracted with 10% MeOH in DCM (2×50 mL). The combined organic layers was dried over Na₂SO₄, filtered, and concentrated under reduced pressure to get the crude N-(3-((tert-butyldimethylsilyl)oxy)-2,6-dichlorophenyl)-4-methoxy-2-((1-methyl-1H-pyrazol-4-yl)amino)pyrimidine-5-carboxamide (200 mg, 0.382 mmol, 88% yield) as black gummy solid. This compound, as such, is taken for the next step without any further purification. LCMS (ESI) m/z=523.1 (M+H), t_{R}: 2.23, 64.20% (Method-D)

### N-(2,6-dichloro-3-hydroxyphenyl)-4-methoxy-2-((1-methyl-1H-pyrazol-4-yl)amino)pyrimidine-5-carboxamide

To a stirred solution of N-(3-((tert-butyldimethylsilyl)oxy)-2,6-dichlorophenyl)-4-methoxy-2-((1-methyl-1H-pyrazol-4-yl)amino)pyrimidine-5-carboxamide (300 mg, 0.573 mmol) in MeOH (5 ml) was added potassium hydrogen fluoride (134 mg, 1.719 mmol) at 0° C. under inert atmosphere. Then, the resulting reaction mixture was stirred at room temperature for 1 h. Progress of the reaction was monitored by TLC and LCMS. The reaction mixture was concentrated under reduced pressure to give the crude compound, which was purified by Prep. HPLC to give N-(2,6-dichloro-3-hydroxyphenyl)-4-methoxy-2-((1-methyl-1H-pyrazol-4-yl)amino)pyrimidine-5-carboxamide (35 mg, 0.084 mmol, 14.63% yield) as an off-white solid. LCMS (ESI) m/z=409.0 (M+H), t_{R}: 1.28, 98.65% (Method-B). ¹H-NMR (400 MHz, DMSO-d₆): δ 10.52 (s, 1H), 10.13 (s, 1H), 9.40 (s, 1H), 8.74-8.70 (m, 1H), 7.95-7.90 (m, 1H), 7.59-7.54 (m, 1H), 7.34-7.32 (m, 1H), 6.98-6.96 (m, 1H), 4.15-4.05 (m, 3H), 3.84 (s, 3H).

### Example 91. Wee1A Kinase Binding Assay

We used a LanthaScreen Europium (Eu) Kinase Binding Assay to determine the binding of provided compounds to Wee1A kinase. The assay utilizes an Alexa Fluor 647-labeled ATP competitive kinase tracer that binds to the ATP binding site of a GST-tagged Wee1A kinase, while a europium (Eu) labeled antibody binds to the GST tag. The proximity of fluorescently labeled kinase tracer and europium (Eu) donor fluorophore antibody leads to fluorescence resonance energy transfer (FRET) to the fluorescence label (acceptor) upon excitation of the Eu (donor). Displacement of tracer from the ATP-binding site by a test compound disturbs the proximity between both labels thus lowering the FRET.

This time resolved-FRET binding assay was performed in white 384-well low volume plates (Greiner, cat #784075), at room temperature in kinase buffer A (KBA; Invitrogen cat #PV3189), consisting of 50 mM HEPES-NaOH (pH 7.5), 0.01% Brij-35, 10 mM MgCl₂, and 1 mM EGTA. 5 μl of compound (diluted in reaction buffer, to 1% DMSO) were added to various wells in the plate, followed by 5 μl each of recombinant human Wee1A kinase (full length Wee1A kinase was expressed by baculovirus in insect cells using a N-terminal GST tag (MW: 99.1 kDa) (Invitrogen cat #PV3817) and LanthaScreen Eu-anti-GST antibody (Invitrogen, cat #PV5594). After this, 5 μl of kinase tracer 178 (Invitrogen, cat #PV5593) was added to the plate and the plate was incubated for 60 minutes at room temperature. The final assay conditions in each well were: 30 nM tracer 178, 5 nM Wee1A kinase and 2 nM Eu-labeled antibody in total assay volume of 15 μl. An Envision 2104 (Perkin-Elmer) Plate Reader with the following time-resolve fluorescence setting was used for performing LanthaScreen kinase binding assay.
Excitation 320 nm (30 nm bandpass)
Kinase Tracer Emission 665 nm (10 nm bandpass)
LanthaScreen Eu-anti-Tag Antibody Emission 615 nm (10 nm bandpass)
Dichroic Mirror Instrument dependent
Delay Time 100 μs
Integration Time 200 μs

To calculate the emission ratio, the acceptor/tracer emission (665 nM) was divided by the antibody/donor emission (615 nM) and the average of duplicate measurement was used for calculations. Data plotting, analysis of binding, and curve fitting was done using Excel add-in XLfit version 5.5.0.5 (IDBS, Guildford, United Kingdom). Results are presented in Table 2, below, where compounds having an IC₅₀ less than or equal to 10 nM are represented as “A”; compounds having an IC₅₀ greater than 10 nM but less than or equal to 100 nM are represented as “B”; compounds having an IC₅₀ greater than 100 nM but less than or equal to 5.00 nM are represented as “C”; and compounds having an IC₅₀ greater than 500 nM are represented as “D”.

TABLE 2
Wee1A kinase Binding IC₅₀ Values for Exemplary Compounds

| Compound | IC₅₀ |
|---|---|
| 100 | A |
| 101 | B |
| 102 | A |
| 103 | B |
| 104 | A |
| 105 | A |
| 106 | A |
| 107 | A |
| 108 | A |
| 109 | B |
| 110 | A |
| 111 | A |
| 112 | B |
| 113 | A |
| 114 | A |
| 115 | B |
| 116 | A |
| 117 | B |
| 118 | B |
| 119 | B |
| 120 | A |
| 121 | A |
| 122 | A |
| 123 | B |
| 124 | C |
| 125 | A |
| 126 | B |
| 127 | A |
| 128 | A |
| 129 | A |
| 130 | A |
| 131 | C |
| 132 | A |
| 133 | A |
| 134 | A |
| 135 | A |
| 136 | A |
| 137 | B |
| 138 | A |
| 139 | B |
| 140 | A |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | B |
| 158 | A |
| 159 | B |
| 160 | B |
| 161 | A |
| 162 | A |
| 163 | A |
| 164 | A |
| 165 | B |
| 166 | A |
| 167 | A |
| 168 | B |
| 169 | C |
| 170 | C |
| 171 | B |
| 172 | A |
| 173 | B |
| 174 | A |
| 175 | A |
| 176 | B |
| 177 | A |
| 178 | A |
| 179 | A |
| 180 | A |
| 181 | A |
| 182 | B |
| 183 | B |
| 184 | A |
| 185 | A |
| 186 | A |
| 187 | A |
| 188 | A |
| 189 | A |
| 190 | A |
| 191 | B |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | A |

### Example 92. Wee1A Kinase Cellular Assays

### Cell Culture

ACHN renal cell carcinoma cell line (ATCC) was cultured in Minimum Essential Medium Eagle supplemented with 10% fetal calf serum (Sigma), 1% Penicillin-Streptomycin and 10 mM HEPES buffer (HyClone). Cell cultures were kept in a humidified incubator at 37° C. and 5% CO₂. Cells were routinely tested for Mycoplasma contamination.

### AlphaLISA Assay

For target engagement assessment, quantification of Cdk1 phosphorylated on tyrosine 15 was detected using pCDK1 Y¹⁵ AlphaLISA assay (Perkin Elmer). ACHN cells were seeded into tissue culture treated 96-well plates (VWR) to a density of 20,000 cells per well. 24 h post seeding cells were treated for 4 h with compounds at concentrations ranging from 7 to 5000 nM. Cells were washed with PBS and lysed in 50 μl alphaLISA lysis buffer before freezing at −80° C. 10 μl of the lysate was transferred to 384 well plates and incubated with alphaLISA donor and acceptor beads according to the manufacturer's instructions. Dose response curves and EC₅₀ values were calculated and visualized using GraphPad Prism version 9.

Results are presented in Table 2A, below, where compounds having an EC₅₀ less than or equal to 500 nM are represented as “A”; compounds having an EC₅₀ greater than 500 nM but less than or equal to 1000 nM are represented as “B”; compounds having an EC₅₀ greater than 1000 nM but less than or equal to 2500 nM are represented as “C”; and compounds having an EC₅₀ greater than 2500 nM are represented as “D”.

### Table 2A. AlphaLISA CDK1 Tyr15 Phosphorylation EC₅₀ Values for Exemplary Compounds.

TABLE 2A
AlphaLISA CDK1 Tyr15 Phosphorylation EC₅₀
Values for Exemplary Compounds.

| Compound | TE Weel (ALISA) |
|---|---|
| 154 | D |
| 155 | A |
| 156 | B |
| 163 | D |
| 171 | D |
| 172 | B |
| 173 | D |
| 174 | D |
| 175 | A |
| 176 | D |
| 177 | D |
| 178 | B |
| 179 | D |
| 180 | B |
| 181 | A |
| 182 | D |

High-Content Imaging of pCdk1 Y¹⁵

High-content imaging of ACHN renal cell carcinoma cells was used for quantification of CDK1 phosphorylated on tyrosine 15 by immunofluorescence (“IF”). 20 000 cells per well were seeded in tissue culture treated 96 well plates and treated with compounds at concentrations ranging from 7 to 5000 nM for 4 h. Cells were fixated for 15 minutes in 4% paraformaldehyde solution, washed in PBS and permeabilized using 0.2% Triton X-100. Blocking was performed for 1 h at RT using Blocker FL Fluorescent Blocking Buffer (Thermo Scientific), thereafter cells were incubated with primary antibody (rabbit anti-pCDK1 Y¹⁵, #4539, Cell Signaling Technology) at 4° C. ON. Alexa Fluor Plus 647 labelled goat anti-rabbit (A32733. Thermo Scientific) was used as secondary antibody. After washing in PBS, nuclei were stained with DAPI solution for 10 min at RT, protected from light. Cells were imaged using an ImageXpress Pico automated imaging system and CellReporter Xpress software (Molecular Devices). The percentage cells with nuclear positivity for pCdk1 Y¹⁵ compared to DMSO control was determined for each well and drug dose response curves and EC₅₀ values were calculated and visualized using GraphPad Prism.

Results are presented in Table 3, below, where compounds having an EC₅₀ less than or equal to 100 nM are represented as “A” compounds having an EC₅₀ greater than 100 nM but less than or equal to 500 nM are represented as “B”; compounds having an EC₅₀ greater than 500 nM but less than or equal to 1000 nM are represented as “C”; and compounds having an EC₅₀ greater than 1000 nM are represented as “D”.

TABLE 3
CDK1 Tyr15 Phosphorylation
EC₅₀ Values for Exemplary Compounds

| Compound | IC₅₀ |
|---|---|
| 100 | A |
| 101 | D |
| 102 | B |
| 104 | B |
| 105 | D |
| 106 | B |
| 107 | D |
| 108 | B |
| 110 | A |
| 111 | B |
| 113 | B |
| 114 | D |
| 116 | A |
| 119 | B |
| 120 | D |
| 121 | B |
| 122 | B |
| 123 | D |
| 125 | D |
| 127 | B |
| 128 | B |
| 129 | A |
| 130 | D |
| 132 | B |
| 133 | A |
| 134 | B |
| 135 | A |
| 136 | B |
| 137 | D |
| 138 | D |
| 140 | B |
| 141 | A |
| 142 | A |
| 143 | B |
| 144 | D |
| 145 | B |
| 146 | A |
| 147 | D |
| 148 | D |
| 149 | B |
| 150 | D |
| 151 | D |
| 152 | B |
| 153 | A |
| 154 | B |
| 157 | B |
| 158 | B |
| 161 | B |
| 162 | B |
| 163 | B |
| 164 | D |
| 165 | D |
| 166 | B |
| 167 | B |
| 172 | B |
| 175 | A |
| 178 | A |
| 179 | B |
| 180 | B |
| 182 | B |
| 184 | B |
| 185 | B |
| 186 | D |
| 187 | D |
| 188 | B |
| 189 | B |
| 190 | B |
| 192 | B |
| 193 | A |
| 194 | B |
| 195 | B |
| 196 | B |
| 197 | B |
| 198 | B |
| 199 | B |
| 200 | B |
| 201 | D |
| 202 | D |
| 203 | D |
| 204 | D |
| 205 | B |
| 206 | B |
| 207 | B |
| 209 | B |
| 210 | B |
| 211 | B |
| 212 | B |
| 213 | B |
| 214 | B |
| 215 | B |
| 216 | B |
| 217 | B |
| 218 | B |
| 219 | B |
| 220 | B |
| 221 | B |
| 222 | B |
| 223 | D |
| 224 | D |

### Cell Titer Glo Viability Assay

ACHN cells were seeded at a density of 400 cells per well in tissue culture treated 384 well plates (Corning Costar). After overnight incubation, cells were treated with drugs at concentrations ranging from 0.17 to 10,000 nM. Viability compared to untreated control was assessed at 144 h using Cell Titer Glo assay 2.0 (Promega).

Results are presented in Table 4, below, where compounds having an absolute EC₅₀ less than or equal to 250 nM are represented as “A”; compounds having an EC₅₀ greater than 250 nM but less than or equal to 1000 nM are represented as “B”; compounds having an EC₅₀ greater than 1000 nM but less than or equal to 5000 nM are represented as “C”; and compounds having an EC₅₀ greater than 5000 nM are represented as “D”.

TABLE 4
ACHN Cell Viability EC₅₀ Values of Exemplary Compounds.

| Compound # | EC₅₀ |
|---|---|
| 100 | B |
| 110 | A |
| 116 | B |
| 122 | B |
| 129 | B |
| 133 | A |
| 135 | B |
| 140 | A |
| 141 | A |
| 158 | B |
| 163 | B |
| 172 | B |
| 175 | A |
| 178 | B |
| 179 | B |
| 180 | B |
| 182 | C |
| 190 | B |
| 194 | B |
| 195 | B |
| 197 | A |
| 200 | B |
| 205 | A |
| 210 | B |
| 212 | A |
| 213 | A |

The relevant teachings of all patents, published applications and references cited herein are incorporated by reference in their entirety.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permit the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound having structural formula I: 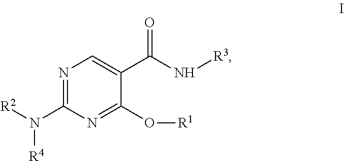
or a solvate, enantiomer, tautomer, or diastereomer thereof, or a pharmaceutically acceptable salt of any of the foregoing wherein:
R¹ is an optionally substituted C₁-C₆ alkyl or an optionally substituted C₃-C₆ cycloalkyl;
R² is an optionally substituted aryl or an optionally substituted heteroaryl, wherein two substituents on the aryl or heteroaryl are optionally taken together to form a saturated ring fused to the aryl or heteroaryl;
R³ is an optionally substituted aryl or an optionally substituted heteroaryl other than tetrazolyl; and
R⁴ is hydrogen or C₁-C₄ alkyl, wherein the compound is other than:

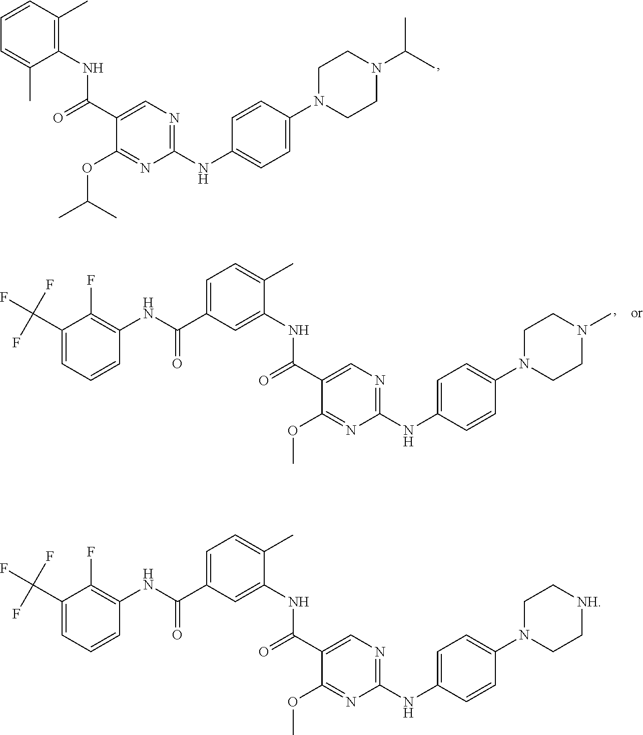 [SMILES: Cc1cccc(C)c1NC(=O)c1cnc(Nc2ccc(N3CCN(C(C)C)CC3)cc2)nc1OC(C)C ; COc1nc(Nc2ccc(N3CCN(C)CC3)cc2)ncc1C(=O)Nc1cc(C(=O)Nc2cccc(C(F)(F)F)c2F)ccc1C ; COc1nc(Nc2ccc(N3CCNCC3)cc2)ncc1C(=O)Nc1cc(C(=O)Nc2cccc(C(F)(F)F)c2F)ccc1C]

2. The compound of claim 1, wherein R¹ is methyl, 2,2,2-trifluoroethyl, 2-hydroxyethyl, cyclopropyl, 3-hydroxypropyl, 3-dimethylaminopropyl, or tetrahydrofuran-2-ylmethyl.

3. The compound of claim 1 or 2, wherein R² is phenyl, pyridinyl, 1H-pyrazolyl, 1H-indazolyl, or 1,2,3,4-tetrahydroisoquinolinyl.

4. The compound of any one of claims 1-3, wherein R² is optionally substituted with one or more substituents independently selected from halo, —CN, C₁-C₆ alkyl, C₁-C₆ haloalkyl, —O—C₁-C₆ alkyl, —O—C₁-C₆ alkylene-N(C₁-C₄ alkyl)₂, heterocyclyl, —O-heterocyclyl, —O—C₁-C₆ alkylene-heterocyclyl, —C₁-C₆ alkylene-heterocyclyl, —C(O)-heterocyclyl, —C₁-C₆ alkylene-N(C₁-C₄ alkyl)₂ and —C₁-C₆ alkylene-NH(C₁-C₄ alkyl), wherein each heterocyclyl portion of a substituent is optionally substituted with up to 3 substituents independently selected from oxo, —CN, and C₁-C₄ alkyl.

5. The compound of claim 4, wherein R² is optionally substituted with one or more substituents independently selected from CN, chloro, fluoro, methyl, methoxy, ethoxy, propoxy, azepan-4-yl, 1,4-diazepan-1-yl, 1-methyl-2-dimethylaminoethan-1-yl, 1-methyl-2-methylaminoethan-1-yl, 1-methyl-4-cyanopiperidin-1-yl, 1-methylazepan-4-yl, 1-methylpiperidin-3-yl, 1-methylpiperidin-4-yl, 1-methylpiperidin-4-yloxy, 1-methylpyrrolidin-3-yl, 1-methylpyrrolidin-3-ylmethyl, 1-methylpyrrolidin-3-yloxy, 1-methylpyrrol-3-yl, 2-(morpholin-4-yl)ethan-1-oxy, 2-dimethylaminoethan-1-oxy, 2-dimethylaminoethan-1-yl, 3-(morpholin-4-yl)propan-1-oxy, 3-dimethylaminopropan-1-yl, 3-dimethylaminopropan-1-oxy, 4-dimethylaminopiperidin-1-yl, 4-methylpiperazin-1-yl, 4-methylpiperazin-1-ylcarbonyl, 4-methyl-7-oxo-1,4-diazepan-1-yl, 4-methyl-1,4-diazepan-1-yl, 7-oxo-1,4-diazepan-1-yl, 9-methyl-3,9-diazaspiro[5.5]undec-3-yl, pyrrolidin-3-yloxy, piperazin-1-yl, piperidin-3-yl, piperidin-4-yl, piperidin-4-yloxy, pyrrolidin-2-ylmethyl, pyrrolidin-3-ylmethyl, and pyrrolidin-3-yl.

6. The compound of claim 3, wherein R² is phenyl, 1-(1H-pyrrolidin-2-yl)methyl-1H-pyrazin-5-yl, 1-(1H-pyrrolidin-3-yl)methyl-1H-pyrazin-5-yl, 1-(1-methyl-1H-pyrrolidin-3-yl)-1 H-indazol-5-yl, 1-(1-methylpiperidin-4-yl)-1H-indazol-5-yl, 1-(1-methylpiperidin-4-yl)pyrazin-5-yl, 1-(1-methylpyrrolidin-3-yl)methylpyrazin-4-yl, 1-(2-dimethylaminoethan-yl)-1H-indazol-5-yl, 1-methyl-1H-indazol-5-yl, 1-methyl-1H-pyrazin-5-yl, 2-(1-methylpiperidin-4-yl)oxy-3-methylpyridin-4-yl, 2,4,4-trimethyl-1,2,3,4-tetrahydroisoquinolin-7-yl, 2-methyl-1,2,3,4-tetrahydroisoquinolin-7-yl, 3-cyano-4-(1-methylpiperidin-4-yl)phenyl, 3-methyl-4-(1-methyl-1H-pyrrolidin-3-yl)oxyphenyl, 3-methyl-4-(1-methyl-4-cyanopiperidin-4-yl)phenyl, 3-methyl-4-(1-methylpiperidin-4-yl)oxyphenyl, 3-methyl-4-(1-methylpiperidin-4-yl)phenyl, 3-methyl-4-(2-dimethylaminoethan-1-yl)oxyphenyl, 3-methyl-4-(2-morpholin-4-ylethan-1-yl)oxyphenyl, 3-methyl-4-(3-dimethylaminopropan-1-yl)oxyphenyl, 3-methyl-4-(3-morpholin-4-ylpropan-1-yl)oxyphenyl, 3-methyl-4-(3-oxo-1,4-diazapin-1-yl)phenyl, 3-methyl-4-(4-methylpiperazin-1-yl)carbonylphenyl, 3-methyl-4-(4-methylpiperazin-1yl)phenyl, 3-methyl-4-piperazin-1-ylphenyl, 3-methyl-4-pyridin-1-ylphenyl, 3-methyl-4-pyridin-4-yloxyphenyl, 3-methyl-4-pyrrolidin-3-yloxyphenyl, 4-(4-methylpiperazin-1-yl)phenyl, 4-piperidin-4-yloxyphenyl, 1-(1-methyl-2,2-dimethylaminoethan-1-yl)pyrazol-4-yl, 1-(1-methyl-2-methylaminoethan-1-yl)pyrazol-4-yl, 1-(1-methylazepan-4-yl)pyrazol-4-yl, 1-(1-methylpiperidin-4-yl)pyrazol-4-yl, 1-azepan-4-ylpyrazol-4-yl, 1-methylpyrazol-4-yl, 3-chloro-4-(1-methylpiperidin-4-yl)phenyl, 3-ethoxy-4-(1-methylpiperidin-4-yl)phenyl, 3-methoxy-4-(1-methylpiperidin-4-yl)phenyl, 3-methyl-4-(1,4-diazepan-1-yl)phenyl, 3-methyl-4-(1-methylazepan-4-yl)phenyl, 3-methyl-4-(1-methylpiperidin-3-yl)phenyl, 3-methyl-4-(1-methylpyrrol-3-yl)phenyl, 3-methyl-4-(1-methylpyrrolidin-3-yl)phenyl, 3-methyl-4-(1-methyl-pyrrolidin-3-yloxy)phenyl, 3-methyl-4-(4-dimethylaminopiperidin-1-yl)phenyl, 3-methyl-4-(piperidin-4-yloxy)phenyl, 3-methyl-4-piperidin-3-ylphenyl, 3-methyl-4-piperidin-4-ylphenyl, 3-methyl-4-pyrrolidin-3-ylphenyl, 3-methyl-5-fluoro-4-(4-dimethylaminopiperidin-1-yl)phenyl, 3-propoxy-4-(1-methylpiperidin-4-yl)phenyl, 4-(1,4-diazepan-1-yl)phenyl, 4-(4-methyl-1,4-diazepan-1-yl)phenyl, 4-(4-methyl-7-oxo-1,4-diazepan-1-yl)phenyl, 4-(9-methyl-3,9-diazaspiro[5.5]undec-3-yl)phenyl, or 5-(1-methylpiperidin-4-yl)pyridin-3-yl.

7. The compound of any one of claims 1-6, wherein R³ is phenyl, thiophenyl, pyridinyl, or 1H-indazolyl.

8. The compound of claim 7, wherein R³ is substituted with 1 to 3 substituents independently selected from methyl, ethyl, fluoro, chloro, bromo, cyclopropyl, —OCH₃, —SCH₃, —NH₂, —NO₂, —CF₃, —CN, —C≡CH, —CH₂—CH═CH₂, —OH, and —NHC(O)CH₃.

9. The compound of claim 7, wherein R³ has the structure: 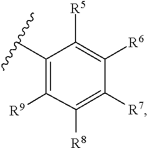
wherein: 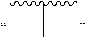
represents a point of attachment of R³ to the compound
R⁵ is hydrogen, chloro, bromo, or methyl;
R⁶ is hydrogen or fluoro;
R⁷ is hydrogen or fluoro;
R⁸ is hydrogen or —OH; and
R⁹ is hydrogen, chloro, fluoro, bromo, methyl, cyclopropyl, —C≡CH, —OCH₃, or —SCH₃, wherein at least one of R⁸ or R⁹ is other than hydrogen.

10. The compound of claim 9, wherein each of R⁵ and R⁹ is other than hydrogen.

11. The compound of claim 9 or 10, wherein each of R⁶ and R⁷ is hydrogen.

12. The compound of any one of claims 9-11, wherein R⁸ is —OH.

13. The compound of claim 7, wherein R³ is 4-chloropyridin-3-yl, 2,6-dichloro-4-fluorophenyl, 2,3-difluoro-6-chlorophenyl, 2,3-difluoro-6-chlorophenyl, 2,4-difluoro-6-chlorophenyl, 2,5-difluoro-6-chlorophenyl, 2-bromo-6-chlorophenyl, 2-bromo-6-fluorophenyl, 2,6-dichloro-3-hydroxyphenyl, 2,6-dichlorophenyl, 2-fluoro-6-chlorophenyl, 2-bromophenyl, 2-chloro-5-hydroxyphenyl, 2-chlorophenyl, 2-chloro-4-methylpyridin-3-yl, 3-hydroxyphenyl, phenyl, 2-bromo-6-cyanophenyl, 2-chloro-6-trifluoromethylphenyl, 2-chloro-5-cyanophenyl, 2-chloro-6-cyanophenyl, 2-trifluoromethylphenyl, 2-cyanophenyl, 2-bromo-6-methylphenyl, 2-methyl-3-hydroxy-6-chlorophenyl, 2-chloro-6-methylphenyl, 2-methyl-5-nitrophenyl, 2,4-dimethylpyridin-3-yl, 2-methyl-5-aminophenyl, 2-chloro-6-ethynylphenyl, 2-ethynylphenyl, 2-methyl-6-trifluoromethylphenyl, 2-chloro-6-ethylphenyl, 2,6-dimethyl-3-hydroxyphenyl, 2-methyl-5-methylcarbamylphenyl, 2,6-dichloro-3-dimethylaminocarbonyloxyphenyl, 5-methyl-1H-indazol-4-yl, 2-bromo-6-ethynylphenyl, or 2,6-dibromophenyl.

14. The compound of claim 1, wherein the compound is any one of the following compounds

| # | Structure |
|---|---|
| 100 | 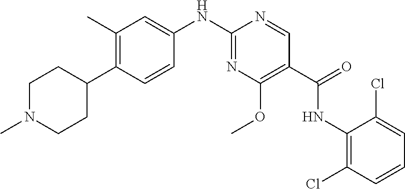 [SMILES: COc1nc(Nc2ccc(C3CCN(C)CC3)c(C)c2)ncc1C(=O)Nc1c(Cl)cccc1Cl] |
| 101 | 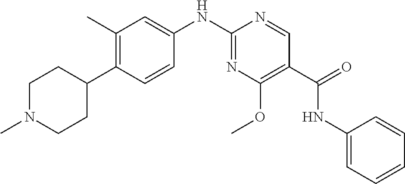 [SMILES: COc1nc(Nc2ccc(C3CCN(C)CC3)c(C)c2)ncc1C(=O)Nc1ccccc1] |
| 102 | 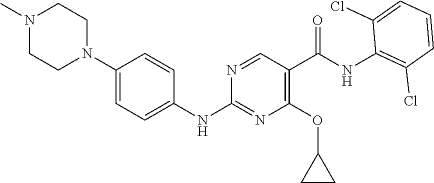 [SMILES: CN1CCN(c2ccc(Nc3ncc(C(=O)Nc4c(Cl)cccc4Cl)c(OC4CC4)n3)cc2)CC1] |
| 103 | 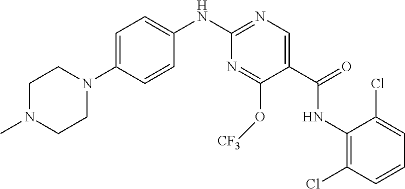 [SMILES: CN1CCN(c2ccc(Nc3ncc(C(=O)Nc4c(Cl)cccc4Cl)c(OC(F)(F)F)n3)cc2)CC1] |
| 104 | 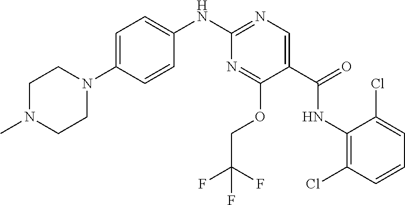 [SMILES: CN1CCN(c2ccc(Nc3ncc(C(=O)Nc4c(Cl)cccc4Cl)c(OCC(F)(F)F)n3)cc2)CC1] |
| 105 | 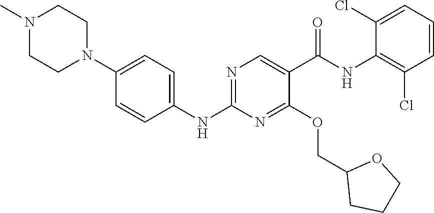 [SMILES: CN1CCN(c2ccc(Nc3ncc(C(=O)Nc4c(Cl)cccc4Cl)c(OCC4CCCO4)n3)cc2)CC1] |
| 106 | 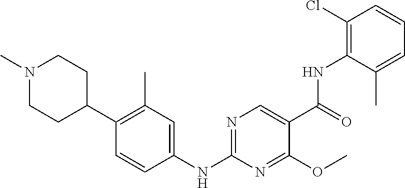 [SMILES: COc1nc(Nc2ccc(C3CCN(C)CC3)c(C)c2)ncc1C(=O)Nc1c(C)cccc1Cl] |
| 107 | 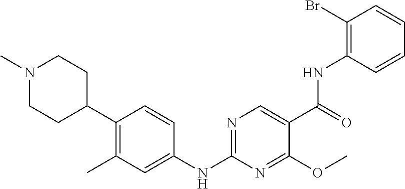 [SMILES: COc1nc(Nc2ccc(C3CCN(C)CC3)c(C)c2)ncc1C(=O)Nc1ccccc1Br] |
| 108 | 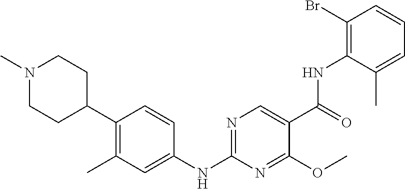 [SMILES: COc1nc(Nc2ccc(C3CCN(C)CC3)c(C)c2)ncc1C(=O)Nc1c(C)cccc1Br] |
| 109 | 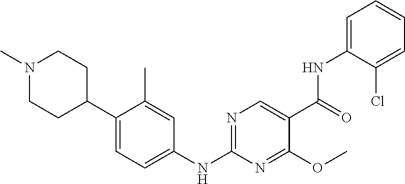 [SMILES: COc1nc(Nc2ccc(C3CCN(C)CC3)c(C)c2)ncc1C(=O)Nc1ccccc1Cl] |
| 110 | 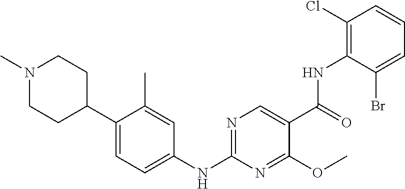 [SMILES: COc1nc(Nc2ccc(C3CCN(C)CC3)c(C)c2)ncc1C(=O)Nc1c(Cl)cccc1Br] |
| 111 | 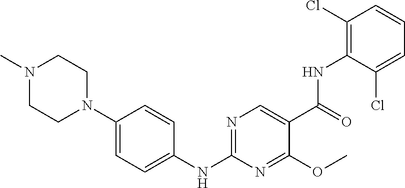 [SMILES: COc1nc(Nc2ccc(N3CCN(C)CC3)cc2)ncc1C(=O)Nc1c(Cl)cccc1Cl] |
| 112 | 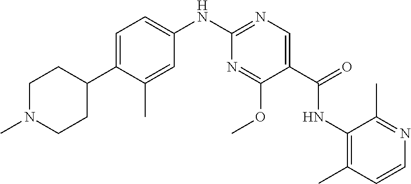 [SMILES: COc1nc(Nc2ccc(C3CCN(C)CC3)c(C)c2)ncc1C(=O)Nc1c(C)ccnc1C] |
| 113 | 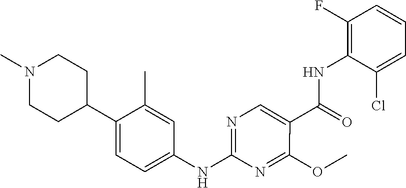 [SMILES: COc1nc(Nc2ccc(C3CCN(C)CC3)c(C)c2)ncc1C(=O)Nc1c(F)cccc1Cl] |
| 114 | 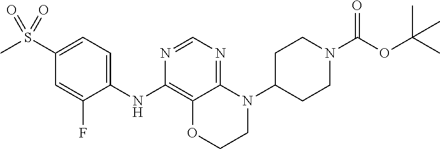 [SMILES: CC(C)(C)OC(=O)N1CCC(N2CCOc3c(Nc4ccc(S(C)(=O)=O)cc4F)ncnc32)CC1] |
| 115 | 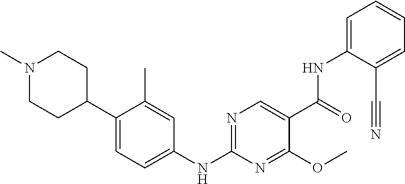 [SMILES: COc1nc(Nc2ccc(C3CCN(C)CC3)c(C)c2)ncc1C(=O)Nc1ccccc1C#N] |
| 116 | 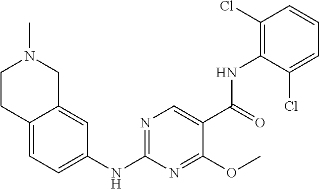 [SMILES: COc1nc(Nc2ccc3c(c2)CN(C)CC3)ncc1C(=O)Nc1c(Cl)cccc1Cl] |
| 117 | 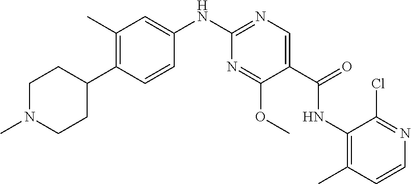 [SMILES: COc1nc(Nc2ccc(C3CCN(C)CC3)c(C)c2)ncc1C(=O)Nc1c(C)ccnc1Cl] |
| 118 | 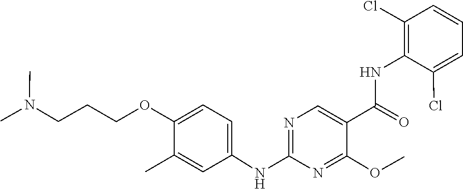 [SMILES: COc1nc(Nc2ccc(OCCCN(C)C)c(C)c2)ncc1C(=O)Nc1c(Cl)cccc1Cl] |
| 119 | 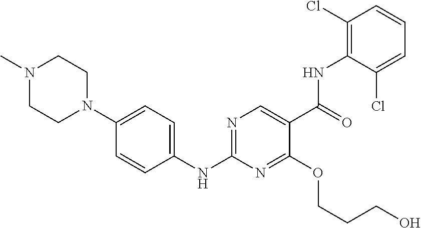 [SMILES: CN1CCN(c2ccc(Nc3ncc(C(=O)Nc4c(Cl)cccc4Cl)c(OCCCO)n3)cc2)CC1] |
| 120 | 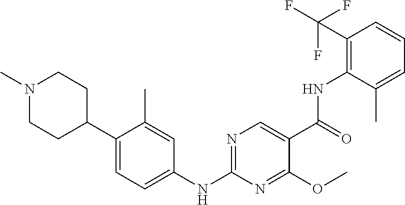 [SMILES: COc1nc(Nc2ccc(C3CCN(C)CC3)c(C)c2)ncc1C(=O)Nc1c(C)cccc1C(F)(F)F] |
| 121 | 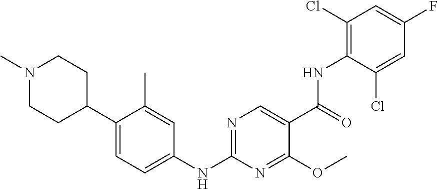 [SMILES: COc1nc(Nc2ccc(C3CCN(C)CC3)c(C)c2)ncc1C(=O)Nc1c(Cl)cc(F)cc1Cl] |
| 122 | 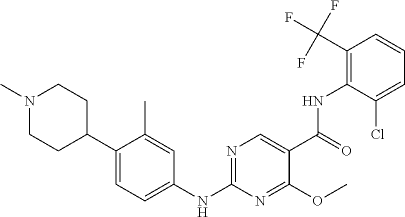 [SMILES: COc1nc(Nc2ccc(C3CCN(C)CC3)c(C)c2)ncc1C(=O)Nc1c(Cl)cccc1C(F)(F)F] |
| 123 | 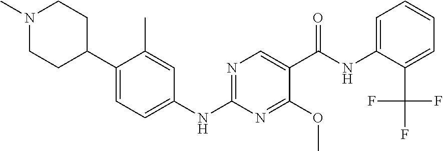 [SMILES: COc1nc(Nc2ccc(C3CCN(C)CC3)c(C)c2)ncc1C(=O)Nc1ccccc1C(F)(F)F] |
| 124 | 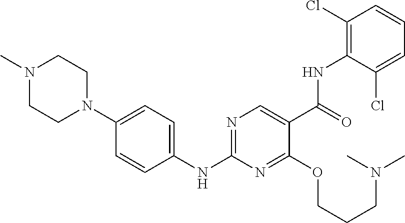 |
| 125 | 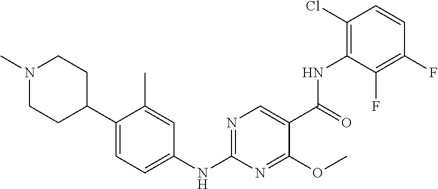 [SMILES: COc1nc(Nc2ccc(C3CCN(C)CC3)c(C)c2)ncc1C(=O)Nc1c(Cl)ccc(F)c1F] |
| 126 | 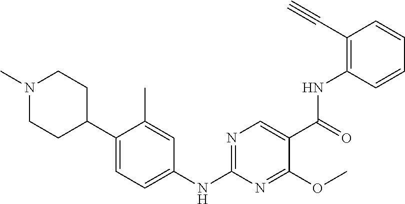 [SMILES: C#Cc1ccccc1NC(=O)c1cnc(Nc2ccc(C3CCN(C)CC3)c(C)c2)nc1OC] |
| 127 | 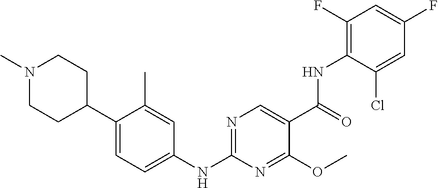 [SMILES: COc1nc(Nc2ccc(C3CCN(C)CC3)c(C)c2)ncc1C(=O)Nc1c(F)cc(F)cc1Cl] |
| 128 | 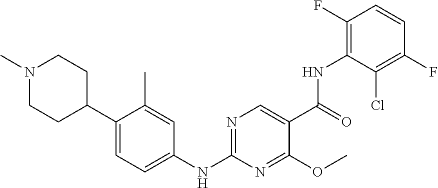 [SMILES: COc1nc(Nc2ccc(C3CCN(C)CC3)c(C)c2)ncc1C(=O)Nc1c(F)ccc(F)c1Cl] |
| 129 | 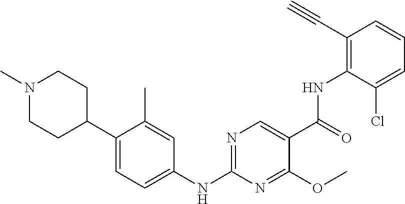 [SMILES: C#Cc1cccc(Cl)c1NC(=O)c1cnc(Nc2ccc(C3CCN(C)CC3)c(C)c2)nc1OC] |
| 130 | 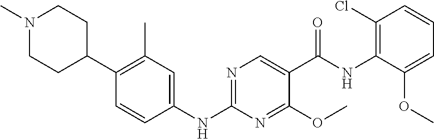 |
| 131 | 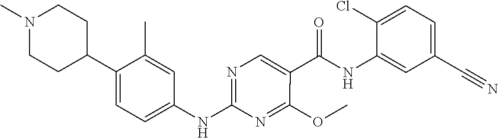 [SMILES: COc1nc(Nc2ccc(C3CCN(C)CC3)c(C)c2)ncc1C(=O)Nc1cc(C#N)ccc1Cl] |
| 132 | 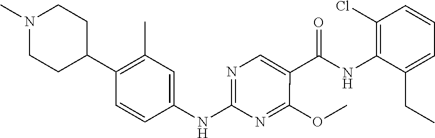 [SMILES: CCc1cccc(Cl)c1NC(=O)c1cnc(Nc2ccc(C3CCN(C)CC3)c(C)c2)nc1OC] |
| 133 | 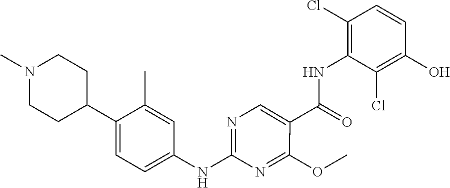 [SMILES: COc1nc(Nc2ccc(C3CCN(C)CC3)c(C)c2)ncc1C(=O)Nc1c(Cl)ccc(O)c1Cl] |
| 134 | 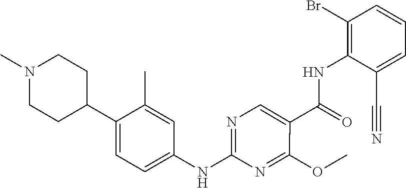 [SMILES: COc1nc(Nc2ccc(C3CCN(C)CC3)c(C)c2)ncc1C(=O)Nc1c(Br)cccc1C#N] |
| 135 | 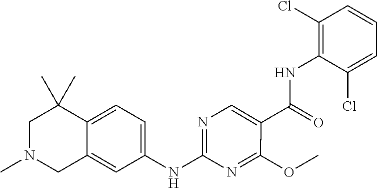 [SMILES: COc1nc(Nc2ccc3c(c2)CN(C)CC3(C)C)ncc1C(=O)Nc1c(Cl)cccc1Cl] |
| 136 | 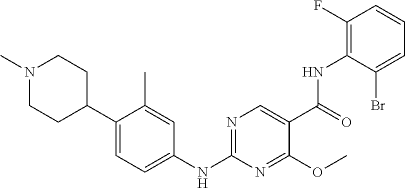 [SMILES: COc1nc(Nc2ccc(C3CCN(C)CC3)c(C)c2)ncc1C(=O)Nc1c(F)cccc1Br] |
| 137 | 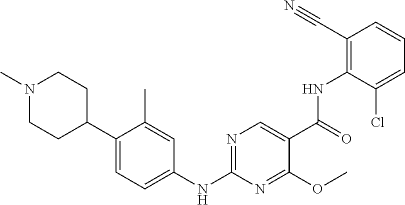 [SMILES: COc1nc(Nc2ccc(C3CCN(C)CC3)c(C)c2)ncc1C(=O)Nc1c(Cl)cccc1C#N] |
| 138 | 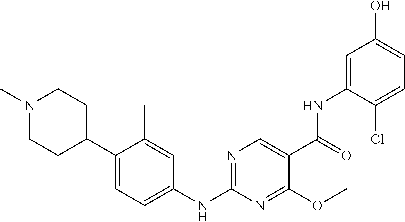 [SMILES: COc1nc(Nc2ccc(C3CCN(C)CC3)c(C)c2)ncc1C(=O)Nc1cc(O)ccc1Cl] |
| 139 | 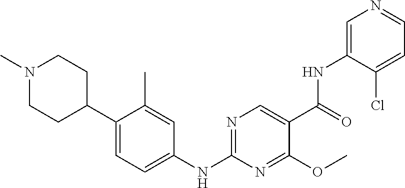 [SMILES: COc1nc(Nc2ccc(C3CCN(C)CC3)c(C)c2)ncc1C(=O)Nc1cnccc1Cl] |
| 140 | 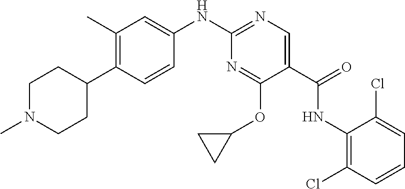 [SMILES: Cc1cc(Nc2ncc(C(=O)Nc3c(Cl)cccc3Cl)c(OC3CC3)n2)ccc1C1CCN(C)CC1] |
| 141 | 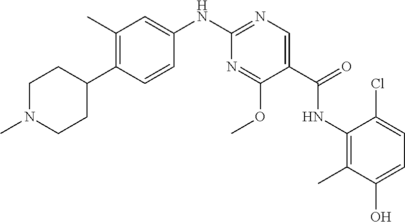 [SMILES: COc1nc(Nc2ccc(C3CCN(C)CC3)c(C)c2)ncc1C(=O)Nc1c(Cl)ccc(O)c1C] |
| 142 | 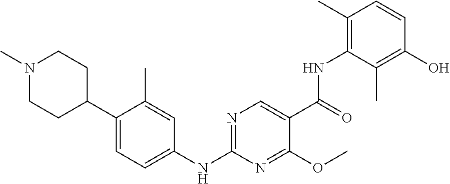 [SMILES: COc1nc(Nc2ccc(C3CCN(C)CC3)c(C)c2)ncc1C(=O)Nc1c(C)ccc(O)c1C] |
| 143 | 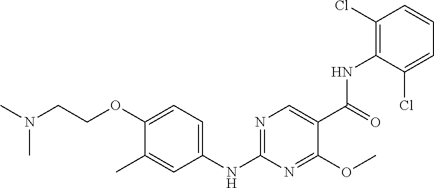 [SMILES: COc1nc(Nc2ccc(OCCN(C)C)c(C)c2)ncc1C(=O)Nc1c(Cl)cccc1Cl] |
| 144 | 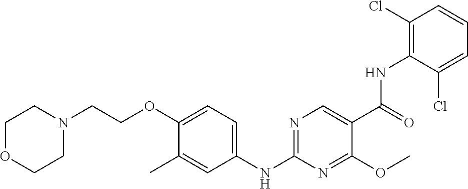 [SMILES: COc1nc(Nc2ccc(OCCN3CCOCC3)c(C)c2)ncc1C(=O)Nc1c(Cl)cccc1Cl] |
| 145 | 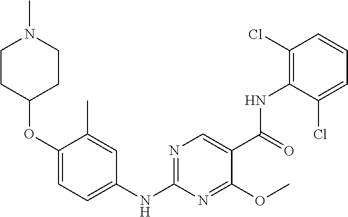 [SMILES: COc1nc(Nc2ccc(OC3CCN(C)CC3)c(C)c2)ncc1C(=O)Nc1c(Cl)cccc1Cl] |
| 146 | 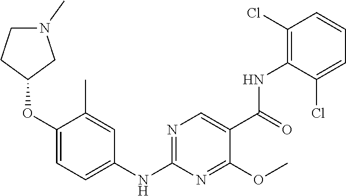 [SMILES: COc1nc(Nc2ccc(O[C@@H]3CCN(C)C3)c(C)c2)ncc1C(=O)Nc1c(Cl)cccc1Cl] |
| 147 | 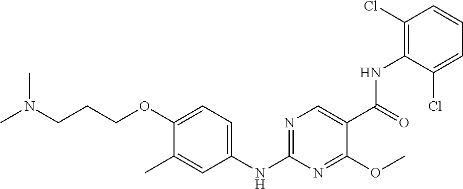 [SMILES: COc1nc(Nc2ccc(OCCCN(C)C)c(C)c2)ncc1C(=O)Nc1c(Cl)cccc1Cl] |
| 148 | 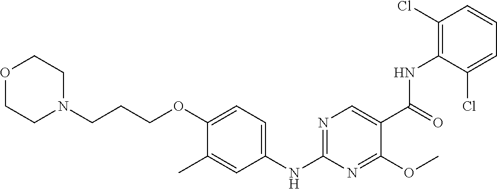 [SMILES: COc1nc(Nc2ccc(OCCCN3CCOCC3)c(C)c2)ncc1C(=O)Nc1c(Cl)cccc1Cl] |
| 149 | 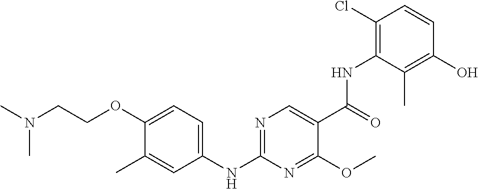 [SMILES: COc1nc(Nc2ccc(OCCN(C)C)c(C)c2)ncc1C(=O)Nc1c(Cl)ccc(O)c1C] |
| 150 | 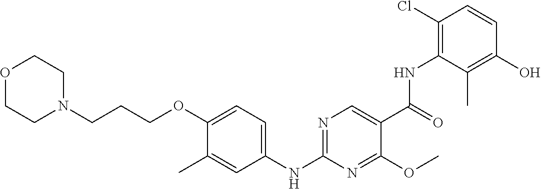 [SMILES: COc1nc(Nc2ccc(OCCCN3CCOCC3)c(C)c2)ncc1C(=O)Nc1c(Cl)ccc(O)c1C] |
| 151 | 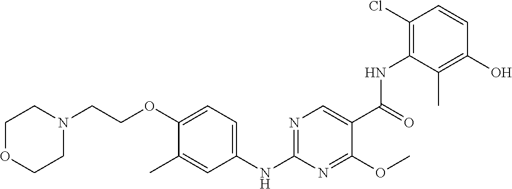 [SMILES: COc1nc(Nc2ccc(OCCN3CCOCC3)c(C)c2)ncc1C(=O)Nc1c(Cl)ccc(O)c1C] |
| 152 | 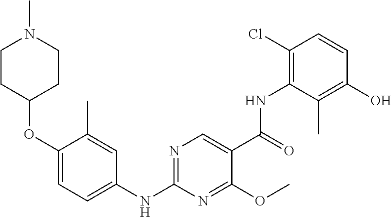 [SMILES: COc1nc(Nc2ccc(OC3CCN(C)CC3)c(C)c2)ncc1C(=O)Nc1c(Cl)ccc(O)c1C] |
| 153 | 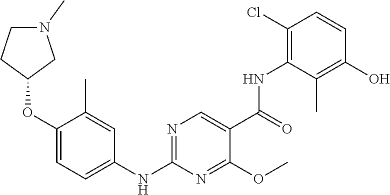 [SMILES: COc1nc(Nc2ccc(O[C@@H]3CCN(C)C3)c(C)c2)ncc1C(=O)Nc1c(Cl)ccc(O)c1C] |
| 154 | 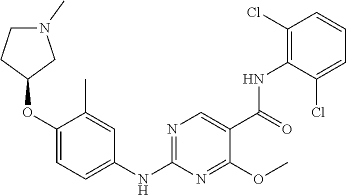 [SMILES: COc1nc(Nc2ccc(O[C@H]3CCN(C)C3)c(C)c2)ncc1C(=O)Nc1c(Cl)cccc1Cl] |
| 155 | 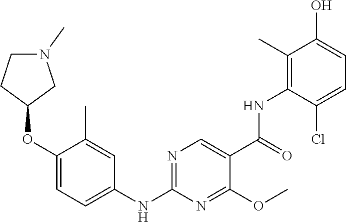 [SMILES: COc1nc(Nc2ccc(O[C@H]3CCN(C)C3)c(C)c2)ncc1C(=O)Nc1c(Cl)ccc(O)c1C] |
| 156 | 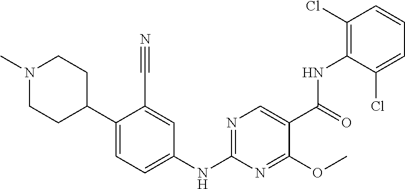 [SMILES: COc1nc(Nc2ccc(C3CCN(C)CC3)c(C#N)c2)ncc1C(=O)Nc1c(Cl)cccc1Cl] |
| 157 | 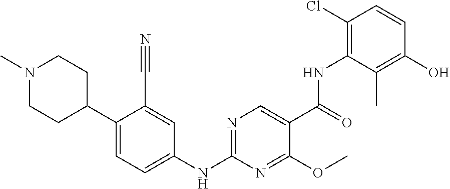 [SMILES: COc1nc(Nc2ccc(C3CCN(C)CC3)c(C#N)c2)ncc1C(=O)Nc1c(Cl)ccc(O)c1C] |
| 158 | 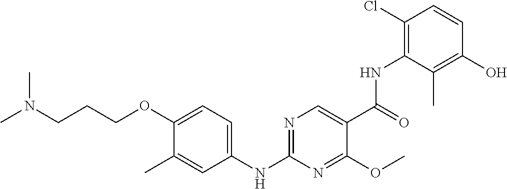 [SMILES: COc1nc(Nc2ccc(OCCCN(C)C)c(C)c2)ncc1C(=O)Nc1c(Cl)ccc(O)c1C] |
| 159 | 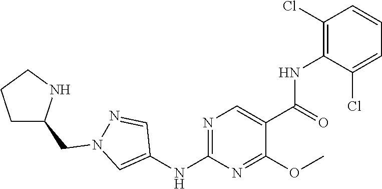 [SMILES: COc1nc(Nc2cnn(C[C@H]3CCCN3)c2)ncc1C(=O)Nc1c(Cl)cccc1Cl] |
| 160 | 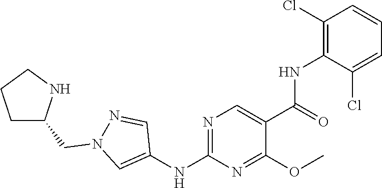 [SMILES: COc1nc(Nc2cnn(C[C@@H]3CCCN3)c2)ncc1C(=O)Nc1c(Cl)cccc1Cl] |
| 161 | 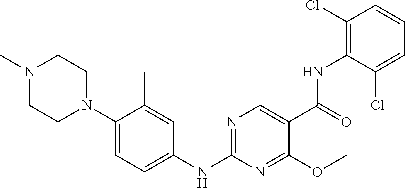 [SMILES: COc1nc(Nc2ccc(N3CCN(C)CC3)c(C)c2)ncc1C(=O)Nc1c(Cl)cccc1Cl] |
| 162 | 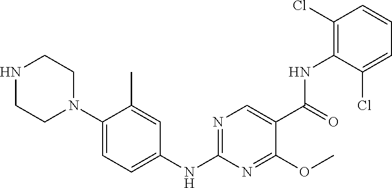 [SMILES: COc1nc(Nc2ccc(N3CCNCC3)c(C)c2)ncc1C(=O)Nc1c(Cl)cccc1Cl] |
| 163 | 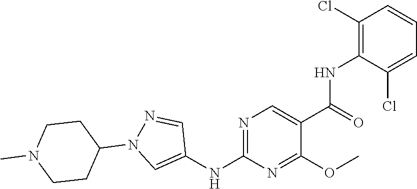 [SMILES: COc1nc(Nc2cnn(C3CCN(C)CC3)c2)ncc1C(=O)Nc1c(Cl)cccc1Cl] |
| 164 | 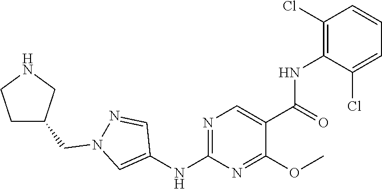 [SMILES: COc1nc(Nc2cnn(C[C@@H]3CCNC3)c2)ncc1C(=O)Nc1c(Cl)cccc1Cl] |
| 165 | 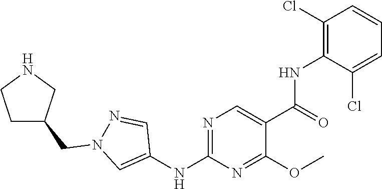 [SMILES: COc1nc(Nc2cnn(C[C@H]3CCNC3)c2)ncc1C(=O)Nc1c(Cl)cccc1Cl] |
| 166 | 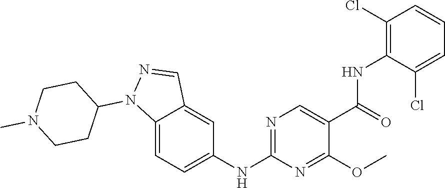 [SMILES: COc1nc(Nc2ccc3c(cnn3C3CCN(C)CC3)c2)ncc1C(=O)Nc1c(Cl)cccc1Cl] |
| 167 | 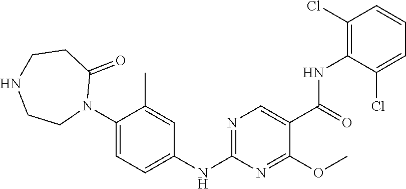 [SMILES: COc1nc(Nc2ccc(N3CCNCCC3=O)c(C)c2)ncc1C(=O)Nc1c(Cl)cccc1Cl] |
| 168 | 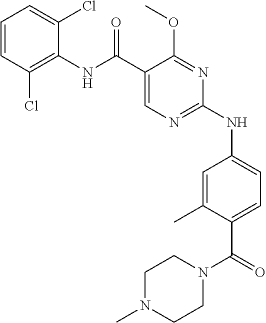 [SMILES: COc1nc(Nc2ccc(C(=O)N3CCN(C)CC3)c(C)c2)ncc1C(=O)Nc1c(Cl)cccc1Cl] |
| 169 | 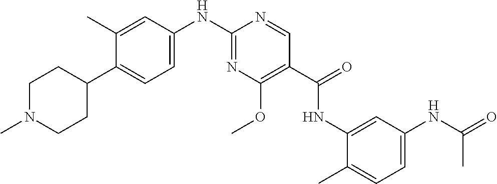 [SMILES: COc1nc(Nc2ccc(C3CCN(C)CC3)c(C)c2)ncc1C(=O)Nc1cc(NC(C)=O)ccc1C] |
| 170 | 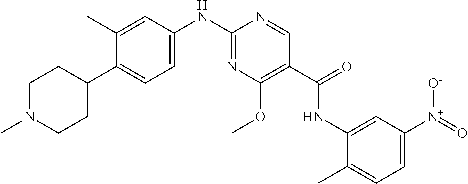 [SMILES: COc1nc(Nc2ccc(C3CCN(C)CC3)c(C)c2)ncc1C(=O)Nc1cc([N+](=O)[O-])ccc1C] |
| 171 | 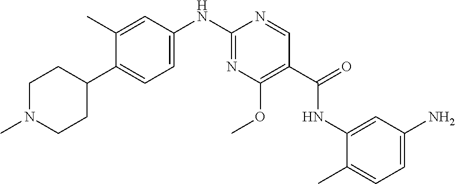 [SMILES: COc1nc(Nc2ccc(C3CCN(C)CC3)c(C)c2)ncc1C(=O)Nc1cc(N)ccc1C] |
| 172 | 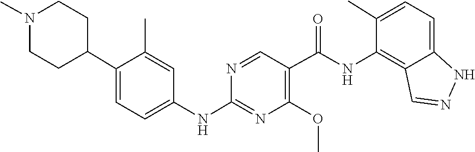 [SMILES: COc1nc(Nc2ccc(C3CCN(C)CC3)c(C)c2)ncc1C(=O)Nc1c(C)ccc2[nH]ncc12] |
| 173 | 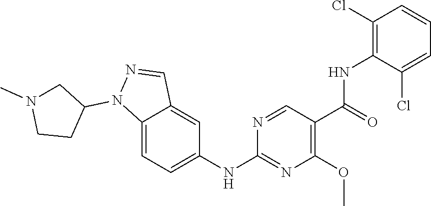 [SMILES: COc1nc(Nc2ccc3c(cnn3C3CCN(C)C3)c2)ncc1C(=O)Nc1c(Cl)cccc1Cl] |
| 174 | 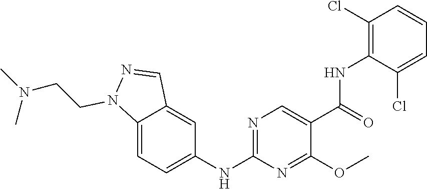 [SMILES: COc1nc(Nc2ccc3c(cnn3CCN(C)C)c2)ncc1C(=O)Nc1c(Cl)cccc1Cl] |
| 175 | 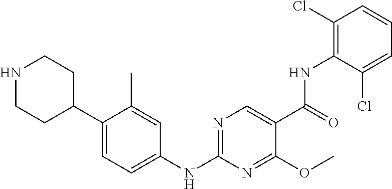 [SMILES: COc1nc(Nc2ccc(C3CCNCC3)c(C)c2)ncc1C(=O)Nc1c(Cl)cccc1Cl] |
| 176 | 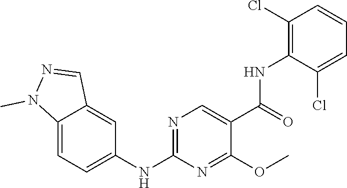 [SMILES: COc1nc(Nc2ccc3c(cnn3C)c2)ncc1C(=O)Nc1c(Cl)cccc1Cl] |
| 177 | 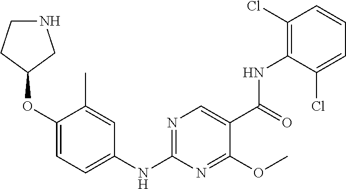 [SMILES: COc1nc(Nc2ccc(O[C@H]3CCNC3)c(C)c2)ncc1C(=O)Nc1c(Cl)cccc1Cl] |
| 178 | 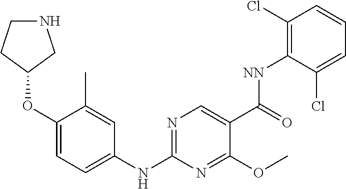 |
| 179 | 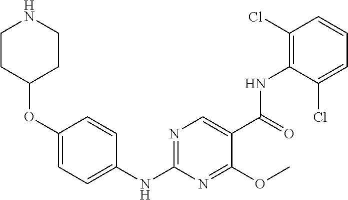 [SMILES: COc1nc(Nc2ccc(OC3CCNCC3)cc2)ncc1C(=O)Nc1c(Cl)cccc1Cl] |
| 180 | 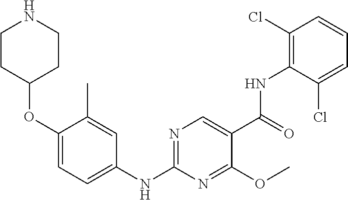 [SMILES: COc1nc(Nc2ccc(OC3CCNCC3)c(C)c2)ncc1C(=O)Nc1c(Cl)cccc1Cl] |
| 181 | 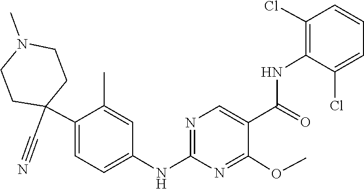 [SMILES: COc1nc(Nc2ccc(C3(C#N)CCN(C)CC3)c(C)c2)ncc1C(=O)Nc1c(Cl)cccc1Cl] |
| 182 | 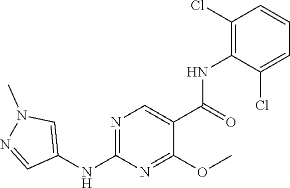 [SMILES: COc1nc(Nc2cnn(C)c2)ncc1C(=O)Nc1c(Cl)cccc1Cl] |
| 183 | 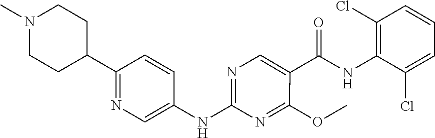 [SMILES: COc1nc(Nc2ccc(C3CCN(C)CC3)nc2)ncc1C(=O)Nc1c(Cl)cccc1Cl] |
| 184 | 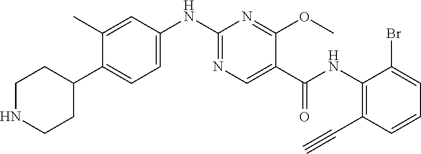 [SMILES: C#Cc1cccc(Br)c1NC(=O)c1cnc(Nc2ccc(C3CCNCC3)c(C)c2)nc1OC] |
| 185 | 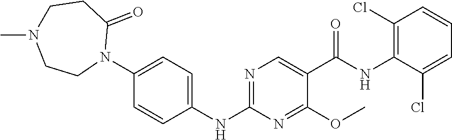 [SMILES: COc1nc(Nc2ccc(N3CCN(C)CCC3=O)cc2)ncc1C(=O)Nc1c(Cl)cccc1Cl] |
| 186 | 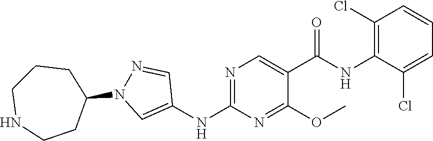 [SMILES: COc1nc(Nc2cnn([C@@H]3CCCNCC3)c2)ncc1C(=O)Nc1c(Cl)cccc1Cl] |
| 187 | 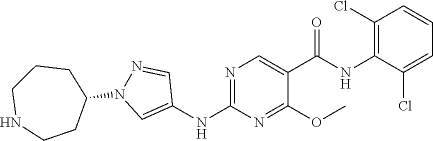 [SMILES: COc1nc(Nc2cnn([C@H]3CCCNCC3)c2)ncc1C(=O)Nc1c(Cl)cccc1Cl] |
| 188 | 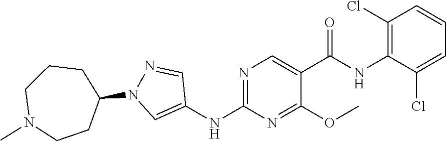 [SMILES: COc1nc(Nc2cnn([C@@H]3CCCN(C)CC3)c2)ncc1C(=O)Nc1c(Cl)cccc1Cl] |
| 189 | 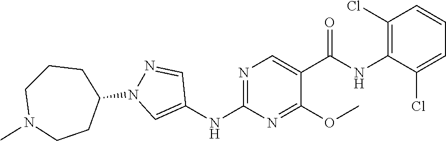 [SMILES: COc1nc(Nc2cnn([C@H]3CCCN(C)CC3)c2)ncc1C(=O)Nc1c(Cl)cccc1Cl] |
| 190 | 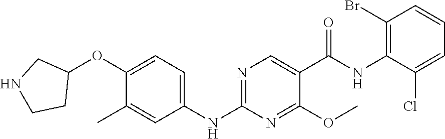 [SMILES: COc1nc(Nc2ccc(OC3CCNC3)c(C)c2)ncc1C(=O)Nc1c(Cl)cccc1Br] |
| 191 | 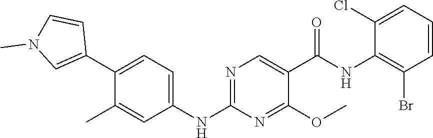 [SMILES: COc1nc(Nc2ccc(-c3ccn(C)c3)c(C)c2)ncc1C(=O)Nc1c(Cl)cccc1Br] |
| 192 | 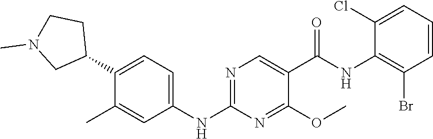 [SMILES: COc1nc(Nc2ccc([C@H]3CCN(C)C3)c(C)c2)ncc1C(=O)Nc1c(Cl)cccc1Br] |
| 193 | 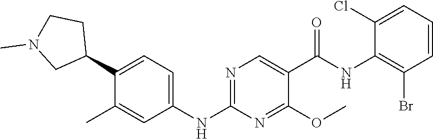 [SMILES: COc1nc(Nc2ccc([C@@H]3CCN(C)C3)c(C)c2)ncc1C(=O)Nc1c(Cl)cccc1Br] |
| 194 | 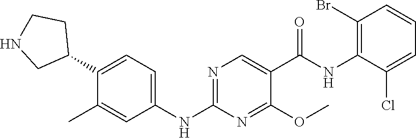 [SMILES: COc1nc(Nc2ccc([C@H]3CCNC3)c(C)c2)ncc1C(=O)Nc1c(Cl)cccc1Br] |
| 195 | 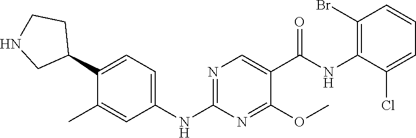 [SMILES: COc1nc(Nc2ccc([C@@H]3CCNC3)c(C)c2)ncc1C(=O)Nc1c(Cl)cccc1Br] |
| 196 | 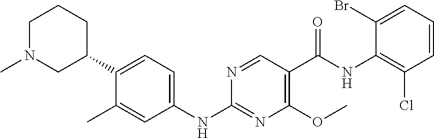 [SMILES: COc1nc(Nc2ccc([C@H]3CCCN(C)C3)c(C)c2)ncc1C(=O)Nc1c(Cl)cccc1Br] |
| 197 | 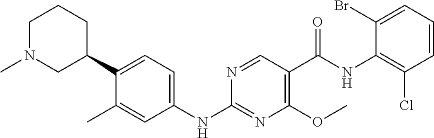 [SMILES: COc1nc(Nc2ccc([C@@H]3CCCN(C)C3)c(C)c2)ncc1C(=O)Nc1c(Cl)cccc1Br] |
| 198 | 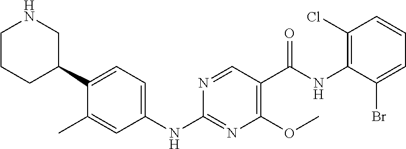 [SMILES: COc1nc(Nc2ccc([C@H]3CCCNC3)c(C)c2)ncc1C(=O)Nc1c(Cl)cccc1Br] |
| 199 | 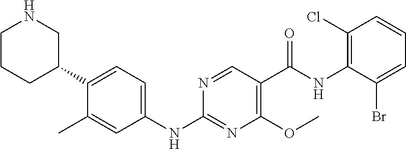 [SMILES: COc1nc(Nc2ccc([C@@H]3CCCNC3)c(C)c2)ncc1C(=O)Nc1c(Cl)cccc1Br] |
| 200 | 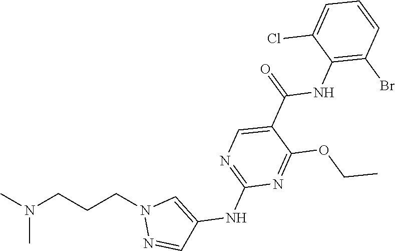 [SMILES: CCOc1nc(Nc2cnn(CCCN(C)C)c2)ncc1C(=O)Nc1c(Cl)cccc1Br] |
| 201 | 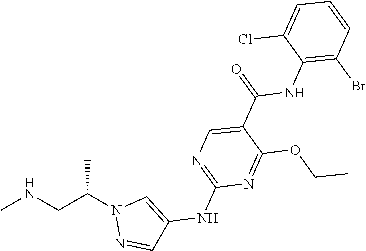 [SMILES: CCOc1nc(Nc2cnn([C@@H](C)CNC)c2)ncc1C(=O)Nc1c(Cl)cccc1Br] |
| 202 | 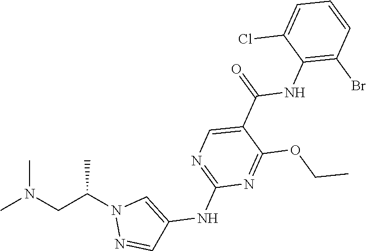 [SMILES: CCOc1nc(Nc2cnn([C@@H](C)CN(C)C)c2)ncc1C(=O)Nc1c(Cl)cccc1Br] |
| 203 | 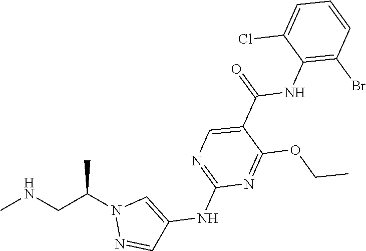 [SMILES: CCOc1nc(Nc2cnn([C@H](C)CNC)c2)ncc1C(=O)Nc1c(Cl)cccc1Br] |
| 204 | 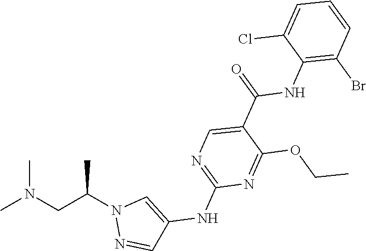 [SMILES: CCOc1nc(Nc2cnn([C@H](C)CN(C)C)c2)ncc1C(=O)Nc1c(Cl)cccc1Br] |
| 205 | 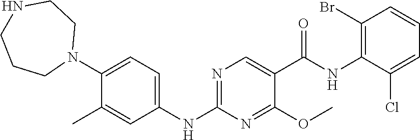 [SMILES: COc1nc(Nc2ccc(N3CCCNCC3)c(C)c2)ncc1C(=O)Nc1c(Cl)cccc1Br] |
| 206 | 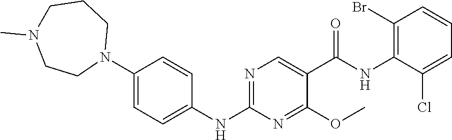 [SMILES: COc1nc(Nc2ccc(N3CCCN(C)CC3)cc2)ncc1C(=O)Nc1c(Cl)cccc1Br] |
| 207 | 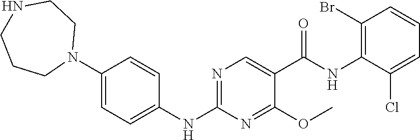 [SMILES: COc1nc(Nc2ccc(N3CCCNCC3)cc2)ncc1C(=O)Nc1c(Cl)cccc1Br] |
| 208 | 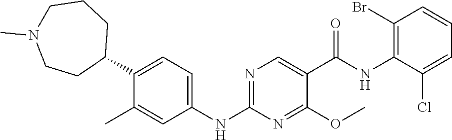 [SMILES: COc1nc(Nc2ccc([C@H]3CCCN(C)CC3)c(C)c2)ncc1C(=O)Nc1c(Cl)cccc1Br] |
| 209 | 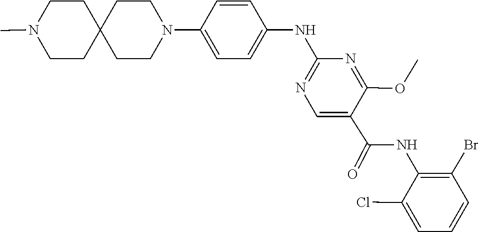 [SMILES: COc1nc(Nc2ccc(N3CCC4(CCN(C)CC4)CC3)cc2)ncc1C(=O)Nc1c(Cl)cccc1Br] |
| 210 | 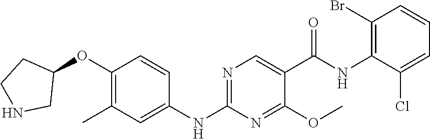 [SMILES: COc1nc(Nc2ccc(O[C@@H]3CCNC3)c(C)c2)ncc1C(=O)Nc1c(Cl)cccc1Br] |
| 211 | 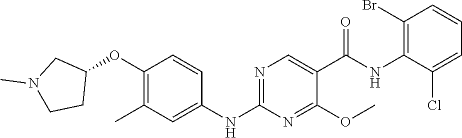 [SMILES: COc1nc(Nc2ccc(O[C@@H]3CCN(C)C3)c(C)c2)ncc1C(=O)Nc1c(Cl)cccc1Br] |
| 212 | 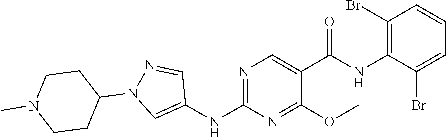 [SMILES: COc1nc(Nc2cnn(C3CCN(C)CC3)c2)ncc1C(=O)Nc1c(Br)cccc1Br] |
| 213 | 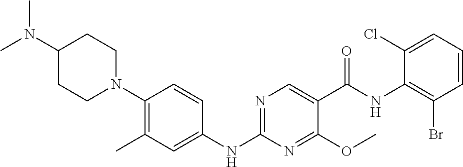 [SMILES: COc1nc(Nc2ccc(N3CCC(N(C)C)CC3)c(C)c2)ncc1C(=O)Nc1c(Cl)cccc1Br] |
| 214 | 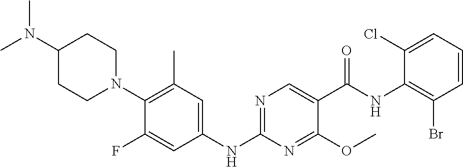 [SMILES: COc1nc(Nc2cc(C)c(N3CCC(N(C)C)CC3)c(F)c2)ncc1C(=O)Nc1c(Cl)cccc1Br] |
| 215 | 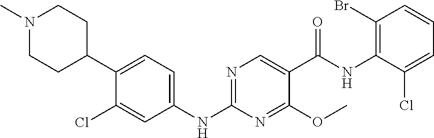 [SMILES: COc1nc(Nc2ccc(C3CCN(C)CC3)c(Cl)c2)ncc1C(=O)Nc1c(Cl)cccc1Br] |
| 216 | 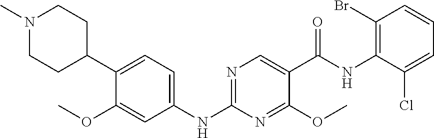 [SMILES: COc1cc(Nc2ncc(C(=O)Nc3c(Cl)cccc3Br)c(OC)n2)ccc1C1CCN(C)CC1] |
| 217 | 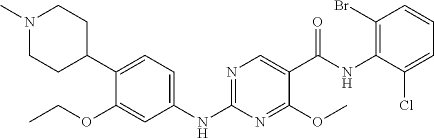 [SMILES: CCOc1cc(Nc2ncc(C(=O)Nc3c(Cl)cccc3Br)c(OC)n2)ccc1C1CCN(C)CC1] |
| 218 | 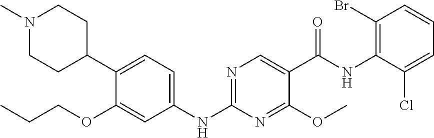 [SMILES: CCCOc1cc(Nc2ncc(C(=O)Nc3c(Cl)cccc3Br)c(OC)n2)ccc1C1CCN(C)CC1] |
| 219 | 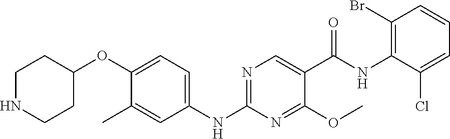 [SMILES: COc1nc(Nc2ccc(OC3CCNCC3)c(C)c2)ncc1C(=O)Nc1c(Cl)cccc1Br] |
| 220 | 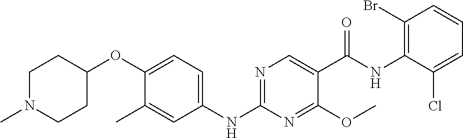 [SMILES: COc1nc(Nc2ccc(OC3CCN(C)CC3)c(C)c2)ncc1C(=O)Nc1c(Cl)cccc1Br] |
| 221 | 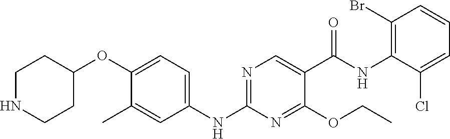 [SMILES: CCOc1nc(Nc2ccc(OC3CCNCC3)c(C)c2)ncc1C(=O)Nc1c(Cl)cccc1Br] |
| 222 | 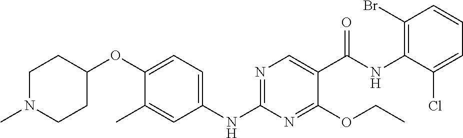 [SMILES: CCOc1nc(Nc2ccc(OC3CCN(C)CC3)c(C)c2)ncc1C(=O)Nc1c(Cl)cccc1Br] |
| 223 | 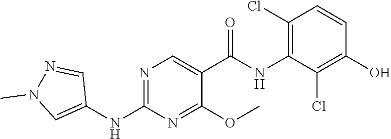 [SMILES: COc1nc(Nc2cnn(C)c2)ncc1C(=O)Nc1c(Cl)ccc(O)c1Cl] |
| 224 | 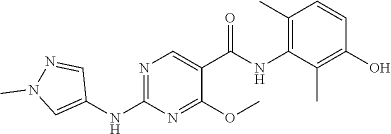 [SMILES: COc1nc(Nc2cnn(C)c2)ncc1C(=O)Nc1c(C)ccc(O)c1C] |
| 225 | 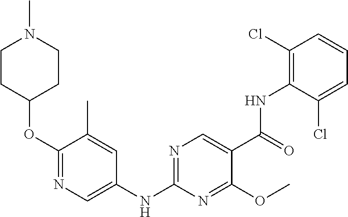 [SMILES: COc1nc(Nc2cnc(OC3CCN(C)CC3)c(C)c2)ncc1C(=O)Nc1c(Cl)cccc1Cl] |
| 226 | 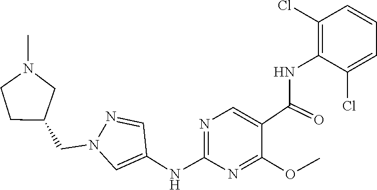 [SMILES: COc1nc(Nc2cnn(C[C@@H]3CCN(C)C3)c2)ncc1C(=O)Nc1c(Cl)cccc1Cl] |
| 227 | 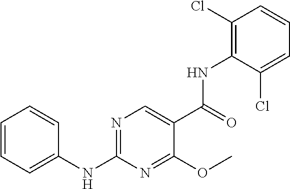 [SMILES: COc1nc(Nc2ccccc2)ncc1C(=O)Nc1c(Cl)cccc1Cl] |
| 228 | 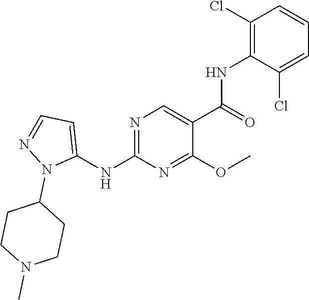 [SMILES: COc1nc(Nc2ccnn2C2CCN(C)CC2)ncc1C(=O)Nc1c(Cl)cccc1Cl] |

or a pharmaceutically acceptable salt thereof.

15. A pharmaceutical composition comprising an effective amount of a compound of any one of claims 1-14; and a pharmaceutically acceptable carrier.

16. A method of inhibiting Wee1A kinase activity in a subject comprising the step of administering to the subject an effective amount of a compound of any one of claims 1-14, or a composition of claim 15.

17. A method of treating a subject suffering from a cancer or other disordered cell growth characterized by aberrant Wee1A kinase activity comprising the step of administering to the subject an effective amount of a compound of any one of claims 1-14, or a composition of claim 15.

18. The method of claim 17, wherein the subject is suffering from a cancer associated with inactivation of p53.

19. The method of claim 17 or 18, wherein the cancer is wherein the cancer is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

20. The method of claim 19, wherein the subject is suffering from a cancer selected from uterine serous carcinoma or a renal cancer.
